(19) United States
(12) Patent Application Publication
(10) Pub. No.: US 2025/0120700 A1
(43) Pub. Date: Apr. 17, 2025

(54) METHOD OF ASSEMBLING A STAPLE CARTRIDGE

(71) Applicant: Cilag GmbH International, Zug (CH)

(72) Inventors: Kevin M. Fiebig, Cincinnati, OH (US); Nicholas Fanelli, Morrow, OH (US); Jeffery D. Bruns, Cincinnati, OH (US); Frederick E. Shelton, IV, Hillsboro, OH (US); Adam D. Hensel, Gahanna, OH (US); Seth D. Holdmeyer, Cincinnati, OH (US); Jason L. Harris, Lebanon, OH (US); Taylor W. Aronhalt, Loveland, OH (US); Carlie J. Love, Loveland, OH (US); Scott A. Jenkins, Mason, OH (US)

(21) Appl. No.: 18/379,790

(22) Filed: Oct. 13, 2023

(51) Int. Cl. A61B 17/072

(52) U.S. Cl. CPC A61B 17/072; A61B 2017/07271; A61B 2017/07278

(57) ABSTRACT

A method of assembling a staple cartridge is disclosed. In various embodiments, the method comprises attaching a pan to a cartridge body, the cartridge body comprising a proximal end, a distal end, a deck, longitudinal rows of staple cavities extending between the proximal end and the distal end, and a longitudinal slot extending from the proximal end toward the distal end. The method further comprises positioning staples in the staple cavities, positioning a support in the longitudinal slot such that an end of the support extends above the deck, and positioning a sled in the cartridge body proximal to the support such that the sled is longitudinally aligned with the support.

### BACKGROUND

The present invention relates to surgical instruments and, in various arrangements, to surgical stapling and cutting instruments and staple cartridges for use therewith that are designed to staple and cut tissue.

### SUMMARY

A method of assembling a staple cartridge is disclosed. In various embodiments, the method comprises attaching a pan to a cartridge body, the cartridge body comprising a proximal end, a distal end, a deck, longitudinal rows of staple cavities extending between the proximal end and the distal end, and a longitudinal slot extending from the proximal end toward the distal end. The method further comprises positioning staples in the staple cavities, positioning a support in the longitudinal slot such that an end of the support extends above the deck, and positioning a sled in the cartridge body proximal to the support such that the sled is longitudinally aligned with the support. In various embodiments, the method comprises obtaining a cartridge body configured to be seated in a jaw of the surgical stapling instrument, positioning a longitudinally-slideable support in a longitudinal slot of the cartridge body such that an end of the support extends above the deck of the cartridge body, and positioning a longitudinally-slideable sled in the cartridge body proximal to the support such that the sled contacts the support during a staple firing stroke and moves the support toward a distal end of the cartridge body during the staple firing stroke. The support is configured to transfer forces to the staple cartridge assembly at a position below the deck of the cartridge body.

### LISTING OF THE FIGURES

Various features of the embodiments described herein, together with advantages thereof, may be understood in accordance with the following description taken in conjunction with the accompanying drawings as follows:

FIG. 1 is a perspective view of a surgical stapling instrument in accordance with at least one embodiment;

FIG. 2 is a partial perspective view of the stapling instrument of FIG. 1;

FIG. 3 is a perspective view of an anvil of the stapling instrument of FIG. 1;

FIG. 4 is a partial plan view of the anvil of FIG. 3;

FIG. 5 is a partial elevational view of the anvil of FIG. 3;

FIG. 6 is a partial elevational view of the anvil of FIG. 3;

FIG. 7 is a partial plan view of the stapling instrument of FIG. 1 illustrated in an open, unclamped configuration;

FIG. 8 is a partial elevational view of the stapling instrument of FIG. 1 illustrated in the open, unclamped configuration of FIG. 7;

FIG. 9 is a cross-sectional view of the stapling instrument of FIG. 1 taken along 9-9 in FIG. 8;

FIG. 10 is a partial plan view of the stapling instrument of FIG. 1 illustrated in a closed, clamped configuration;

FIG. 11 is a partial elevational view of the stapling instrument of FIG. 1 illustrated in the closed, clamped configuration of FIG. 10;

FIG. 12 is a cross-sectional view of the stapling instrument of FIG. 1 taken along 12-12 in FIG. 11;

FIG. 13 is an elevational view of a surgical end effector comprising an anvil shown in phantom lines in an open position in accordance with at least one embodiment;

FIG. 14 is a cross-sectional end view of the surgical end effector of FIG. 13;

FIG. 15 is a partial cross-sectional perspective view of the anvil of the end effector of FIG. 13;

FIG. 16 is a side elevational view of an anvil closure member of the surgical end effector of FIG. 13;

FIG. 17 is a side elevational view of a firing member of the surgical end effector of FIG. 13;

FIG. 18 is a partial side elevational view of the surgical end effector of FIG. 13 with the anvil shown in an open position in phantom lines;

FIG. 19 is another partial side elevational view of the surgical end effector of FIG. 13 with the anvil shown in a closed position;

FIG. 20 is a side elevational view of the surgical end effector of FIG. 13 with the firing member at the beginning of a staple firing stroke;

FIG. 21 is a side elevational view of the surgical end effector of FIG. 13 with the anvil closure member and the firing member being partially distally deployed in the end effector;

FIG. 22 is a perspective view of a distal end of a stapling instrument comprising an end effector in accordance with at least one embodiment;

FIG. 23 is a perspective view of the end effector of FIG. 22 illustrating a replaceable staple cartridge removed from a cartridge jaw of the end effector;

FIG. 23A is a perspective view of the end effector of FIG. 22 without the replaceable staple cartridge;

FIG. 24 is an elevational view of the end effector of FIG. 22 illustrating an anvil jaw of the end effector in an open position;

FIG. 25 is an elevational view of the end effector of FIG. 22 illustrating the anvil jaw of FIG. 24 in a closed position;

FIG. 26 is an elevational view of a firing driver of the stapling instrument of FIG. 22 and a sled, staple driver, and staple of the staple cartridge of FIG. 23;

FIG. 27 is a partial perspective view of the staple cartridge of FIG. 23;

FIG. 28 is a perspective view of the staple driver of FIG. 26;

FIG. 29 is a partial cross-sectional elevational view of the staple cartridge of FIG. 23;

FIG. 30 is a partial cross-sectional elevational view of a staple cartridge in accordance with at least one embodiment;

FIG. 31 is a cross-sectional elevational view of a staple driver in accordance with at least one embodiment;

FIG. 32 is a cross-sectional elevational view of a staple driver in accordance with at least one embodiment;

FIG. 33 is a cross-sectional elevational view of a staple driver being lifted by a sled in accordance with at least one embodiment;

FIG. 34 is a cross-sectional elevational view of a staple driver being lifted by a sled in accordance with at least one embodiment;

FIG. 35 is an elevational view of a staple driver in accordance with at least one embodiment;

FIG. 36 is a cross-sectional view of the staple driver of FIG. 35;

FIG. 37 is an elevational view of a staple driver in accordance with at least one embodiment;

FIG. 38 is a cross-sectional view of the staple driver of FIG. 37;

FIG. 39 is a perspective view of a surgical stapling end effector comprising a shaft, a first jaw, and a second jaw movable relative to the first jaw to clamp tissue between the first jaw and the second jaw;

FIG. 40 is a partial perspective view of a proximal end of a staple cartridge of the surgical stapling end effector of FIG. 39, wherein the staple cartridge is configured to be installed into the first jaw;

FIG. 41 is a partial cross-sectional view of the surgical stapling end effector of FIG. 39 where the staple cartridge is installed in the first jaw and the second jaw is positioned in a fully clamped position;

FIG. 42 is a partial perspective view of a staple cartridge for use with a surgical stapling end effector, wherein the staple cartridge comprises a plurality of outer staple cavities and a plurality of staple drivers configured to eject staples from the outer staple cavities, and wherein each outer staple cavity comprises a retention feature configured to hold one of the staple drivers in an unfired position;

FIG. 43 is a cross-sectional view of the staple cartridge of FIG. 42, wherein the staple drivers are illustrated in their unfired position;

FIG. 44 is a cross-sectional view of a staple cartridge assembly comprising a cartridge body and a sled, wherein the cartridge body comprises an inner cartridge wall comprising a varying height along the longitudinal length of the inner cartridge wall;

FIG. 45 is a perspective view of the sled of the staple cartridge assembly of FIG. 44;

FIG. 46 is a front view of the sled of FIG. 45;

FIG. 47 is a partial cross-sectional view of a proximal zone of the staple cartridge assembly of FIG. 44, where the sled is in a proximal, unfired position;

FIG. 48 is a partial cross-sectional view of an intermediate zone of the staple cartridge assembly of FIG. 44, where the sled is positioned within the intermediate zone;

FIG. 49 is a top view of a staple cartridge assembly comprising a cartridge body having a longitudinal slot and a pre-positioned cartridge support pillar positioned within the longitudinal slot;

FIG. 50 is a perspective view of the cartridge support pillar of FIG. 49;

FIG. 51 is a cross-sectional view of the staple cartridge assembly of FIG. 49;

FIG. 52 is a perspective view of a cartridge support pillar for use with a staple cartridge assembly, wherein the cartridge support pillar comprises staple deployment ramps configured to fire the distal-most staples of a staple cartridge assembly;

FIG. 53 is a cross-sectional view of a staple cartridge assembly comprising a cartridge body, a sled, and a pre-positioned cartridge support pillar within a longitudinal slot of the cartridge body, wherein the sled is in a proximal, unfired position;

FIG. 54 is a perspective view of the cartridge support pillar of FIG. 53;

FIG. 55 is a cross-sectional view of the staple cartridge assembly of FIG. 53, wherein the sled is engaged with the pre-positioned cartridge support pillar;

FIG. 56 is a cross-sectional view of the staple cartridge assembly of FIG. 53, wherein the sled and the pre-positioned cartridge support pillar are positioned in a distal, fired position;

FIG. 56A is a side view of a stapling system comprising a staple cartridge, a cartridge channel, and a movable cartridge support positioned within the staple cartridge, wherein the stapling system further comprises a sensing system configured to sense a parameter of the stapling system;

FIG. 56B is a perspective view of the movable cartridge support of FIG. 56A;

FIG. 56C is a side view of the movable cartridge support of FIG. 56A;

FIG. 57 is a partial perspective view of a surgical stapling system comprising a firing assembly, a sled, a cartridge channel, and a flex circuit configured to detect the position of the sled during a firing stroke;

FIG. 58 is a partial, cross-sectional, perspective view of a surgical stapling assembly comprising a sled, a cartridge channel, and a sled detection system configured to detect the position of the sled during a firing stroke;

FIG. 59 is a schematic view of the surgical stapling assembly of FIG. 58, wherein the sled is in a proximal, unfired position and a conductive post of the sled detection system is in a pre-positioned home position in a proximal zone of the surgical stapling assembly;

FIG. 60 is a schematic view of the surgical stapling assembly of FIG. 58, wherein the sled is engaged with the conductive post in the pre-positioned home position;

FIG. 61 is a schematic view of the surgical stapling assembly of FIG. 58, wherein the sled is engaged with the conductive post in a plurality of fired positions;

FIG. 62 is a schematic view of the surgical stapling assembly of FIG. 58, wherein the sled is retracted into the proximal, unfired position and the conductive post is left in a distal end of the cartridge channel;

FIG. 63 is a perspective view of a surgical stapling assembly comprising a firing driver, a cartridge channel, and a staple cartridge assembly, wherein the staple cartridge assembly comprises a deployable support positioned within a longitudinal slot of the staple cartridge assembly;

FIG. 64 is a top view of the staple cartridge assembly of FIG. 63;

FIG. 65 is a side view of the staple cartridge assembly of FIG. 63;

FIG. 66 is a top view of a sled for use with a surgical stapling assembly, wherein the sled comprises a central portion comprising a proximal end and a distal end, and wherein distal end is thicker than the proximal end;

FIG. 66A is a top view of a staple cartridge assembly for use with the sled of FIG. 66;

FIG. 67 is a partial perspective view of a surgical stapling assembly comprising a staple cartridge assembly, wherein the staple cartridge assembly comprises a cartridge body and a sled, and wherein the cartridge body comprises a plurality of support pillars configured to be hinged out of the way by the sled during a firing stroke;

FIG. 68 is a cross-sectional view of the surgical stapling assembly of FIG. 67, wherein the surgical stapling assembly further comprises a cartridge channel comprising an opposing support surface and a cavity through which the sled is configured to travel during the firing stroke;

FIG. 69 is a cross-sectional view of the surgical stapling assembly of FIG. 67, wherein a pair of opposing support pillars are in a fully-extended configuration;

FIG. 70 is a cross-sectional view of the surgical stapling assembly of FIG. 67, wherein the pair of opposing support pillars shown in FIG. 69 are in a fully-collapsed configuration;

FIG. 71 is a perspective view of a surgical stapling assembly comprising a shaft, a cartridge channel jaw configured to receive a staple cartridge assembly therein, and an anvil jaw in accordance with at least one embodiment;

FIG. 72 is a cross-sectional view of the surgical stapling assembly of FIG. 71 illustrating inner cartridge walls, intermediate cartridge walls, and outer cartridge walls of a staple cartridge seated in the cartridge channel jaw;

FIG. 73 is a partial top view of a cartridge body and a staple driver of a staple cartridge in accordance with at least one embodiment;

FIG. 74 is a partial bottom view of the staple cartridge of FIG. 73;

FIG. 75 is a partial cross-sectional perspective view of a staple cartridge comprising a cartridge body, a sled, and a support insert positioned within a longitudinal slot of the cartridge body in accordance with at least one embodiment;

FIG. 76 is a cross-sectional view of a surgical stapling assembly comprising the staple cartridge assembly of FIG. 75 and a distal I-beam head of a firing driver;

FIG. 77 is a cross-sectional view of a staple cartridge comprising a cartridge body, a sled movable through the cartridge body, and support inserts positioned within a longitudinal slot of the cartridge body in accordance with at least one embodiment;

FIG. 78 is a perspective view of one of the support inserts of FIG. 77;

FIG. 79 is a perspective view of a surgical stapling assembly comprising a shaft, a cartridge channel jaw configured to receive a staple cartridge therein, and an anvil jaw movable relative to the cartridge channel jaw in accordance with at least one embodiment;

FIG. 80 is a perspective view of a staple cartridge for use with the surgical stapling assembly of FIG. 79 in accordance with at least one embodiment, wherein the staple cartridge comprises a cartridge body, a plurality of staples, a plurality of staple drivers, and a sled;

FIG. 81 is a perspective view of a proximal end of the staple cartridge of FIG. 80, wherein the staple cartridge further comprises a plurality of retention features configured to prevent the staple drivers from falling out of the bottom of the cartridge body and configured to guide the sled as the sled is moved distally through a staple firing stroke;

FIG. 82 is a cross-sectional end view of the proximal end of the surgical stapling assembly of FIG. 79 illustrated with some components removed;

FIG. 83 is a perspective view of one of the staple drivers of the staple cartridge of FIG. 80;

FIG. 84 is a perspective view of the sled of the staple cartridge of FIG. 80;

FIG. 85 is a perspective view of a proximal end of a staple cartridge in accordance with at least one embodiment comprising a retention feature configured to hold a sled in an unfired position and configured to guide the sled during a portion of the firing stroke;

FIG. 86 is a perspective view of a staple cartridge in accordance with at least one embodiment comprising a cartridge body, a sled, and a staple retainer configured to hold the sled in an unfired position prior to removal of the staple retainer from the cartridge body;

FIG. 87 is a cross-sectional view of the staple cartridge of FIG. 86, wherein the staple retainer comprises a central fin positioned within a longitudinal slot of the cartridge body and the central fin comprises a proximal hook;

FIG. 88 is a partial perspective view of a proximal end of the staple cartridge of FIG. 86;

FIG. 89 is a perspective view of a proximal end of a staple cartridge in accordance with at least one embodiment comprising a cartridge body and a sled positioned within the cartridge body in an unfired position, wherein the cartridge body comprises retention features engaged with the sled to hold the sled in the unfired position;

FIG. 90 is a bottom view of the proximal end of the staple cartridge of FIG. 89;

FIG. 91 is a cross-sectional view of the staple cartridge of FIG. 89 taken along line 91-91 in FIG. 90;

FIG. 92 is a perspective view of the sled of the staple cartridge of FIG. 89;

FIG. 93 is an end view of a surgical stapling assembly comprising a cartridge channel and a staple cartridge installed within the cartridge channel in accordance with at least one embodiment, wherein the staple cartridge comprises a cartridge body and a sled;

FIG. 94 is a partial perspective view of the staple cartridge of FIG. 93, wherein the cartridge body comprises outer walls each comprising a retention feature extending laterally inward toward a longitudinal slot of the cartridge body to hold the sled in an unfired position;

FIG. 95 is a partial perspective view of a stapling attachment for use with a surgical instrument depicting a staple cartridge positioned in an end effector of the stapling attachment, in accordance with at least one embodiment;

FIG. 96 is an exploded view of the stapling attachment of FIG. 95, depicting the staple cartridge separated from the end effector;

FIG. 97 is a partial perspective view of the stapling attachment of FIG. 95 depicting the end effector of the stapling attachment in an open configuration;

FIG. 98 is a partial cross-sectional view of the stapling attachment of FIG. 95 depicting the end effector in the open configuration;

FIG. 99 is a partial cross-sectional view of the stapling attachment of FIG. 95 depicting the end effector in a closed configuration;

FIG. 100 is a partial perspective view of a firing driver of the stapling attachment of FIG. 95 and a sled of the staple cartridge of FIG. 95;

FIG. 101 is a partial perspective view of a staple cartridge depicting staple drivers and a sled in a partially fired position in accordance with at least one embodiment;

FIG. 102 is a partial perspective view of the staple cartridge of FIG. 101 with the sled in a partially retracted position;

FIG. 103 is a front perspective view of one of the staple drivers of the staple cartridge of FIG. 101;

FIG. 104 is back perspective view of the staple driver of FIG. 103;

FIG. 105 is a side elevational view of the staple driver of FIG. 103;

FIG. 106 is a partial perspective view of a staple cartridge depicting a sled of the staple cartridge in a partially fired position in accordance with at least one embodiment;

FIG. 107 is a partial perspective view of the staple cartridge of FIG. 106 with the sled in a partially retracted position;

FIG. 108 is a partial cross-sectional side view of a staple cartridge illustrated with components removed depicting a sled of the staple cartridge in a first retracted position in accordance with at least one embodiment;

FIG. 109 is a partial cross-sectional side view of the staple cartridge of FIG. 108 illustrated with components removed with the sled in a second retracted position;

FIG. 110 is a partial cross-sectional side view of the staple cartridge of FIG. 108 illustrated with components removed with the sled in a third retracted position;

FIG. 111 is a perspective view of the sled of the staple cartridge of FIG. 108;

FIG. 112 is a partial cross-sectional view of a staple cartridge for use with a surgical instrument depicting a sled assembly of the staple cartridge in a proximal unfired position in accordance with at least one embodiment;

FIG. 113 is a partial cross-sectional view of the staple cartridge of FIG. 112 depicting the sled assembly in an intermediate position distal to the proximal unfired position;

FIG. 114 is a partial cross-sectional view of the staple cartridge of FIG. 112 depicting the sled assembly in a distal fired position;

FIG. 115 is a partial cross-sectional view of the staple cartridge of FIG. 112 depicting the sled assembly retracted proximally from the distal fired position;

FIG. 116 is an exploded view of the sled assembly of the staple cartridge of FIG. 112 depicting a distal driver lift cam;

FIG. 117 is a partial cross-sectional view of a surgical instrument comprising an end effector and a firing driver depicting a staple cartridge received in the end effector and a sled of the staple cartridge in a proximal unfired position in accordance with at least one embodiment;

FIG. 118 is a partial cross-sectional view of the surgical instrument of FIG. 117 depicting the sled advanced to a distal fired position by the firing driver;

FIG. 119 is a partial cross-sectional view of the surgical instrument of FIG. 117 depicting a portion of the retraction stroke where the firing driver is retracted and the sled is expanded;

FIG. 120 is a partial cross-sectional view of the surgical instrument of FIG. 117 depicting the firing driver disengaging the expanded sled and entering a lockout opening of the end effector upon retraction;

FIG. 121 is a partial cross-sectional view of a surgical instrument comprising an end effector and a firing driver depicting a staple cartridge received in the end effector with a sled of the staple cartridge in an unfired position and the firing driver in a proximal position in accordance with at least one embodiment;

FIG. 122 is a partial cross-sectional view of the surgical instrument of FIG. 121 depicting the firing driver advanced from the proximal position toward the staple cartridge;

FIG. 123 is a partial cross-sectional view of the surgical instrument of FIG. 121 depicting the firing driver engaged with the sled of the staple cartridge;

FIG. 124 is a partial cross-sectional view of the surgical instrument of FIG. 121 depicting the firing driver operably coupled to the sled of the staple cartridge;

FIG. 125 is a partial cross-sectional view of the surgical instrument of FIG. 121, depicting the firing driver and sled retracted proximally during a retraction stroke after the firing stroke is completed;

FIG. 126 is a partial cross-sectional view of the surgical instrument of FIG. 121 depicting the firing driver operably de-coupled from the sled of the staple cartridge;

FIG. 127 is a partial cross-sectional view of the surgical instrument of FIG. 121 depicting the firing driver retracted proximal to the sled after the firing driver is de-coupled from the sled;

FIG. 128 is a partial cross-sectional view of the surgical instrument of FIG. 121 depicting the firing driver retracted to the proximal position;

FIG. 129 is a partial perspective view of an end effector of a surgical stapling instrument with a staple cartridge installed therein, in accordance with at least one embodiment;

FIG. 130 is a partial cross-sectional view of a portion of the staple cartridge and end effector of FIG. 129 with a sled of the staple cartridge in an unfired position and a firing driver of the surgical stapling instrument in a starting position;

FIG. 131 is another partial cross-sectional view of the staple cartridge and end effector of FIG. 130 depicting the firing driver advanced from a starting position in FIG. 130 toward the staple cartridge to engage the sled and defeat a driver lockout of the end effector;

FIG. 132 is another partial cross-sectional view of the staple cartridge and end effector of FIG. 130 depicting the firing driver and the sled retracted proximally as a result of a retraction stroke;

FIG. 133 is another partial cross-sectional view of the staple cartridge and end effector of FIG. 130 depicting the firing driver operably disengaged from the sled during the retraction stroke;

FIG. 134 is a partial cross-sectional perspective view of the staple cartridge and end effector of FIG. 130 depicting a flexible tab, detent, or latch of the staple cartridge holding the sled in the unfired position;

FIG. 135 is another partial cross-sectional perspective view of the staple cartridge and end effector of FIG. 130 wherein the flexible tab, detent, or latch of the staple cartridge is preventing the sled from being retracted proximally beyond a released position that is distal to the unfired position;

FIG. 136 is a perspective view of a staple cartridge comprising a sled including a knife in accordance with at least one embodiment with the knife illustrated in an exposed configuration;

FIG. 137 is a perspective view of the sled of the staple cartridge of FIG. 136;

FIG. 138 is a cross-sectional perspective view of the staple cartridge of FIG. 136 depicting the sled in a proximal unfired position and the knife of the sled in a retracted configuration;

FIG. 139 is a cross-sectional perspective view of a proximal portion of the staple cartridge of FIG. 136 depicting the knife of the sled engaging a ledge defined within a longitudinal slot of the staple cartridge;

FIG. 140 is another cross-sectional perspective view of the proximal portion of the staple cartridge of FIG. 136 depicting the knife of the sled in its exposed configuration;

FIG. 141 is a cross-sectional perspective view of the staple cartridge of FIG. 136 depicting the sled in a partially-fired position and the knife of the sled in its exposed configuration;

FIG. 142 is a cross-sectional perspective view of the staple cartridge of FIG. 136 depicting the knife of the sled positioned at a distal end of the ledge of FIG. 139;

FIG. 143 is a cross-sectional perspective view of the staple cartridge of FIG. 136 depicting the sled in a distal fired position and the knife of the sled in its retracted configuration;

FIG. 144 is a cross-sectional perspective view of the staple cartridge of FIG. 136 depicting the sled in its proximal unfired position and the knife of the sled in its retracted configuration;

FIG. 145 is a partial cross-sectional plan view of a staple cartridge and end effector of a surgical stapling instrument with a firing driver of the surgical stapling instrument in a starting position and a sled of the staple cartridge in a proximal unfired position;

FIG. 146 is another partial cross-sectional plan view of the staple cartridge and effector of FIG. 145 after the firing driver has been distally driven into latching engagement with the sled;

FIG. 147 is another partial cross-sectional plan view of the staple cartridge and end effector of FIG. 145 depicting the sled advanced distally from its proximal unfired position by the firing driver;

FIG. 148 is a partial cross-sectional plan view of the staple cartridge and end effector of FIG. 145 depicting the sled in a proximal released position after having been retracted by the firing driver during a retraction stroke;

FIG. 149 is a partial exploded perspective view of the sled and the firing driver of the surgical stapling instrument of FIG. 145;

FIG. 150 is a plan view of the sled of the staple cartridge of FIG. 145 in accordance with at least one embodiment;

FIG. 151 is a partial perspective view of the staple cartridge and end effector of FIG. 145 illustrating a portion of the firing driver in cross section;

FIG. 152 is a partial perspective view of a firing driver and a sled assembly of a surgical stapling instrument having a knife portion and a wedge portion in accordance with at least one embodiment;

FIG. 153 is a cross-sectional elevation view of the firing driver and sled assembly of FIG. 152;

FIG. 154 is a cross-sectional plan view of the firing driver and sled assembly of FIG. 152;

FIG. 155 is a cross-sectional view of an end effector including a staple cartridge and the firing driver and sled assembly of FIG. 152 depicting the sled assembly ejecting staples from the staple cartridge during a staple firing stroke;

FIG. 156 is a cross-sectional view of the end effector of FIG. 155 depicting the sled assembly in a distal fired position after the staple firing stroke has been completed;

FIG. 157 is a cross-sectional view of the end effector of FIG. 155 depicting a wedge portion of the sled assembly disengaging from the firing driver during a retraction stroke of the firing driver;

FIG. 158 is a perspective view of a staple cartridge for use with a surgical stapling instrument in accordance with at least one embodiment, wherein the staple cartridge comprises a longitudinal slot comprising recesses and protrusions;

FIG. 158A is a partial cross-sectional perspective view of the staple cartridge of FIG. 158 illustrating a sled assembly of the staple cartridge including a wedge component and a knife component.

FIG. 159 is a perspective view of the sled assembly of FIG. 158A depicting the knife component of the sled assembly disengaged from the wedge component of the sled assembly;

FIG. 160 is a perspective view of the sled assembly of FIG. 158A depicting the knife component of the sled assembly engaged with the wedge component of the sled assembly;

FIG. 161 is a perspective view of a multi-component sled for use with a staple cartridge in accordance with at least one embodiment;

FIG. 162 is an exploded view of the multi-component sled of FIG. 161;

FIG. 163 is a cross-sectional view of the multi-component sled of FIG. 161;

FIG. 164 is a bottom perspective view of the multi-component sled of FIG. 161;

FIG. 164A is a perspective view of a multi-material sled in accordance with at least one embodiment;

FIG. 165 is a partial perspective view of a staple cartridge comprising a sled in accordance with at least one embodiment depicting the sled in a distal fired position;

FIG. 166 is a partial cross-sectional view of an end effector of a surgical instrument including the staple cartridge of FIG. 165;

FIG. 167 is a perspective view of the sled of the staple cartridge of FIG. 165;

FIG. 168 is another perspective view of the sled of the staple cartridge of FIG. 165;

FIG. 169 is a cross-sectional view of the sled of the staple cartridge of FIG. 165 depicting a knife of the sled in an exposed configuration;

FIG. 170 is a cross-sectional view of the sled of the staple cartridge of FIG. 164 depicting the knife of the sled in a retracted configuration;

FIG. 171 is a cross-sectional view of an end effector comprising a sled of a staple cartridge including a knife housing in accordance with at least one embodiment;

FIG. 172 is a cross-sectional view of the end effector of FIG. 171 depicting a knife of the sled positioned at least partially within the knife housing;

FIG. 173 is a perspective view of the staple cartridge of FIG. 171 depicting the knife of the sled of the staple cartridge positioned at least partially within the knife housing;

FIG. 174A is a partial cross-sectional view of a staple cartridge comprising a sled and a separate tissue cutting knife in accordance with at least one embodiment;

FIG. 174B is a partial cross-sectional view of the staple cartridge of FIG. 174A illustrating the tissue cutting knife in a recessed configuration at the end of a staple firing stroke;

FIG. 175 is a partial perspective view of a surgical stapling system including an end effector;

FIG. 176 is a perspective view of the end effector of FIG. 175 with a staple cartridge removed therefrom;

FIG. 177 is another perspective view of the end effector of FIG. 175 with the staple cartridge removed;

FIG. 178 is an elevational view of the end effector of FIG. 175 in a closed position;

FIG. 179 is an elevational view of the end effector of FIG. 175 in an open position;

FIG. 180 is a perspective view of a surgical stapling system that includes a lockout;

FIG. 181 is a side elevational view of the surgical stapling system of FIG. 180 with the lockout in a locked state;

FIG. 182 is another side elevational view of the surgical stapling system of FIG. 180 with the lockout in an unlocked state;

FIG. 183 is a top view of a surgical stapling system with a compatible and ready-to-fire staple cartridge removably positioned in an end effector with two lockouts in their respective unlocked states;

FIG. 184 is the top of the surgical stapling system of FIG. 183 without a staple cartridge removably positioned in the end effector and the two lockouts in their respective locked states;

FIG. 185 is a partial perspective view of the surgical stapling system of FIG. 184;

FIG. 186 illustrates a surgical stapling system that includes a closure lockout;

FIG. 187 is a partial perspective view of a surgical stapling system that includes a lockout that has been placed into an unlocked state by a compatible staple cartridge;

FIG. 188 is a partial perspective view of the surgical stapling system of FIG. 187 with the lockout in a locked state;

FIG. 189 is a partial perspective view of the surgical stapling system of FIG. 187 with the lockout in another locked state;

FIG. 190 is a partial perspective view of a surgical stapler including an end effector;

FIG. 191 is an exploded perspective view of the end effector and staple cartridge of FIG. 190;

FIG. 192 is another perspective view of the end effector of FIG. 190 with the staple cartridge removed;

FIG. 193 is an elevational view of the end effector of FIG. 190 in a closed position;

FIG. 194 is an elevational view of the end effector of FIG. 190 in an open position;

FIG. 195 is a partial perspective view of the staple cartridge, a firing assembly, and a lockout of the surgical stapler of FIG. 190;

FIG. 196 is a perspective view of the lockout of FIG. 195;

FIG. 197 is a top cross-sectional view of the staple cartridge, the firing assembly, and the lockout of FIG. 195;

FIG. 198 is a partial perspective view of a surgical stapling system including an end effector;

FIG. 199 is a perspective view of the end effector of FIG. 198 with a staple cartridge removed therefrom;

FIG. 200 is another perspective view of the end effector of FIG. 198 with the staple cartridge removed;

FIG. 201 is an elevational view of the end effector of FIG. 198 in a closed position;

FIG. 202 is an elevational view of the end effector of FIG. 198 in an open position;

FIG. 203 illustrates a lockout system comprising a first lockout and a second lockout for using with the surgical stapling system of 198;

FIG. 204 illustrates a top view of the lockout system of FIG. 203 with a compatible staple cartridge being inserted into the end effector;

FIG. 205 illustrates the lockout system of FIG. 203 with the staple cartridge of FIG. 204 transitioning the lockout system to an unlocked state;

FIG. 206 illustrates the lockout system of FIG. 203 with a staple cartridge unlocking the first lockout but not the second lockout;

FIG. 207 illustrates the lockout system of FIG. 203 with a staple cartridge unlocking the second lockout but not the first lockout;

FIG. 208 illustrates the lockout system of FIG. 203 and a firing driver having longitudinally staggered lock shoulders;

FIG. 209 illustrates the system of FIG. 208 with a staple cartridge positioned in the end effector that has unlocked both the first lockout and the second lockout of the lockout system of FIG. 203;

FIG. 210 illustrates the system of FIG. 209 with the firing driver moving through the staple cartridge;

FIG. 211 illustrates the system of FIG. 208 with a staple cartridge unlocking the second lockout of the lockout system but not the first lockout;

FIG. 212 illustrates the system of FIG. 208 with a staple cartridge unlocking the first lockout of the lockout system but not the second lockout;

FIG. 213 illustrates the system of FIG. 208 and the staple cartridge of FIG. 212 with the lockout system preventing distal motion of the firing driver after a first portion of its stroke;

FIG. 214 illustrates the lockout system of FIG. 203 with a firing driver having an elongate lock window;

FIG. 215 illustrates the system of FIG. 214 with a compatible staple cartridge positioned in the end effector that has unlocked the first lockout and the second lockout of the lockout system;

FIG. 216 illustrates the system of FIG. 215 with the firing driver moving through the staple cartridge;

FIG. 217 illustrates the system of FIG. 214 with a staple cartridge that has unlocked the second lockout of the lockout system but not the first lockout;

FIG. 218 illustrates the system of FIG. 214 with a staple cartridge that has unlocked the first lockout of the lockout system but not the second lockout;

FIG. 219 illustrates the system of FIG. 214 and the staple cartridge of FIG. 218 with the lockout system preventing distal motion of the firing driver after a first portion of its stroke;

FIG. 220 illustrates a lockout system and a firing actuator for use with the surgical stapling system of FIG. 198;

FIG. 221 illustrates the lockout system of FIG. 220 in a locked configuration;

FIG. 222 illustrates a perspective view of a surgical stapling system, according to one aspect of the present disclosure;

FIG. 223 illustrates an exploded view of the surgical stapling system of FIG. 222, according to one aspect of the present disclosure;

FIG. 224 illustrates a block diagram of a surgical stapling system, according to one aspect of the present disclosure;

FIG. 225 illustrates a block diagram of a surgical stapling system, according to one aspect of the present disclosure;

FIG. 226 is a graph that illustrates the relationship between firing force over time for various firing strokes of firing drivers;

FIG. 227 is a graph that illustrates exemplary firing strokes of a firing driver for various sizes of staple cartridges and the amount of co-operation that exists between a closure system and a firing system over the firing stroke of the firing driver;

FIG. 228 is a graph that illustrates firing force thresholds for each of the respective exemplary staple cartridges of FIG. 227 over the firing stroke of the firing driver;

FIG. 228A is a method for controlling a surgical stapling instrument, according to one aspect of the present disclose;

FIG. 229 is a graph that illustrates firing force and firing force thresholds over a firing stroke of a firing driver;

FIG. 230 is a graph that illustrates closure load applied by a closure driver over the firing stroke of the firing driver from FIG. 229;

FIG. 231 is a graph that illustrates velocity of the firing driver over the firing stroke of the firing driver from FIG. 229;

FIG. 231A is a method for controlling a surgical stapling instrument, according to one aspect of the present disclose;

FIG. 232 is graphs for an exemplary firing stroke of a firing driver though a staple cartridge;

FIG. 233 is graphs for another exemplary firing stroke of a firing driver though a staple cartridge;

FIG. 233A is a method for controlling a surgical stapling instrument, according to one aspect of the present disclose;

FIG. 234 illustrates a firing driver and an anvil, according to one aspect of the present disclosure;

FIG. 235 is graphs for an exemplary firing stroke of the firing driver of FIG. 234 though the anvil of FIG. 234;

FIG. 235A is a method for controlling a surgical stapling instrument, according to one aspect of the present disclose;

FIG. 236 illustrates a surgical stapling system, according to one aspect of the present disclosure;

FIG. 237 illustrates a side view of a firing driver and firing bar from FIG. 236, according to one aspect of the present disclosure;

FIG. 238 is a graph that illustrates the relationship between firing force over time for various firing strokes of firing drivers for varying tissue thicknesses;

FIG. 239 is a partial perspective view of a surgical stapling system including an end effector;

FIG. 240 is an exploded perspective view of the end effector of FIG. 239;

FIG. 241 is perspective view of an adjunct supported on a deck of a staple cartridge;

FIG. 242 is a perspective view of an adjunct supported adjacent to a staple-forming undersurface of an anvil of a surgical stapler;

FIG. 243 is a top view of a staple cartridge for use with a surgical instrument in accordance with at least one embodiment;

FIG. 243A is a diagrammatic view of a second staple cavity in relation to a portion of a longitudinal slot of the staple cartridge of FIG. 243;

FIG. 244 is a top view of a sled, staple drivers, and staples corresponding to the second staple cavities of the staple cartridge of FIG. 243;

FIG. 245 is a perspective view of the sled, staple drivers, and staples of FIG. 244;

FIG. 246 is a perspective view of reinforced staple lines during subsequent staple firings;

FIG. 247 is a top view of an adjunct for use with a staple cartridge and/or anvil in accordance with at least one embodiment;

FIG. 248 is a cross-sectional side view of the adjunct of FIG. 247;

FIG. 249 is a cross-sectional side of an adjunct in accordance with at least one embodiment;

FIG. 250 is a top view of another adjunct for use with a staple cartridge and/or anvil in accordance with at least one embodiment;

FIG. 251 is a cross-sectional side view of the adjunct of FIG. 250;

FIG. 252 is a cross-sectional side of an adjunct in accordance with at least one embodiment;

FIG. 253 is a top view of an adjunct for use with a staple cartridge and/or anvil in accordance with at least one embodiment;

FIG. 254 is another top view of the adjunct of FIG. 253 in relation to tissue stops of a surgical staple anvil;

FIG. 255 is a top view of another adjunct for use with a staple cartridge and/or anvil in accordance with at least one embodiment, with staple pockets of a staple cartridge shown in phantom lines for reference;

FIG. 256 is a top view of another adjunct for use with a staple cartridge and/or anvil in accordance with at least one embodiment showing some formed staples from a staple cartridge for reference;

FIG. 257 is a top view of another adjunct for use with a staple cartridge and/or anvil in accordance with at least one embodiment;

FIG. 258 is a top view of another adjunct for use with a staple cartridge and/or anvil in accordance with at least one embodiment;

FIG. 259 is a top view of another adjunct for use with a staple cartridge and/or anvil in accordance with at least one embodiment;

FIG. 260 is a diagram of a control system in accordance with at least one embodiment;

FIG. 261 is an algorithm configured for use with the control system of FIG. 260 in accordance with at least one embodiment;

FIG. 262 is a perspective view of a surgical stapler end effector;

FIG. 263 is a cross-sectional side view of an adjunct for use with a staple cartridge and/or anvil of a surgical stapler end effector in accordance with at least one embodiment;

FIG. 264 is a side view of two of the adjuncts of FIG. 263 used in connection with an anvil and a staple cartridge of a surgical stapler end effector to clamp a portion of target tissue therebetween in accordance with at least one embodiment;

FIG. 265 is a cross-sectional side view of another adjunct in accordance with at least one embodiment;

FIG. 266 is a cross-sectional side view of another adjunct in accordance with at least one embodiment;

FIG. 267 is a cross-sectional side view of another adjunct in accordance with at least one embodiment;

FIG. 268 is a cross-sectional side view of another adjunct used in connection with a surgical staple cartridge of a surgical stapler end effector in accordance with at least one embodiment;

FIG. 269 is a cross-sectional side view of another adjunct used in connection with a surgical staple cartridge of a surgical stapler end effector in accordance with at least one embodiment;

FIG. 270 is a cross-sectional side view of another adjunct used in connection with an anvil of a surgical stapler end effector in accordance with at least one embodiment;

FIG. 271 is a partial perspective view of a surgical stapler end effector in accordance with at least one embodiment;

FIG. 272 is a cross-sectional end view of the surgical stapler end effector of FIG. 271 in accordance with at least one embodiment;

FIG. 273 is partial cross-sectional side view of the surgical stapler end effector of FIG. 270 in accordance with at least one embodiment;

FIG. 274 is another partial perspective view of the surgical stapler end effector of FIG. 270 with an adjunct supported on the staple cartridge of the end effector in accordance with at least one embodiment;

FIG. 275 is a perspective view of an anvil and an adjunct of a surgical stapler end effector in accordance with at least one embodiment;

FIG. 276 is a partial cross-sectional perspective view of proximal end portions of the anvil and adjunct of FIG. 275 in accordance with at least one embodiment;

FIG. 277 is a top partial cross-sectional view of a portion of the adjunct and anvil of FIG. 275 used in connection with a surgical stapler end effector with the knife of the end effector in an initial position prior to commencement of a cutting and firing process in accordance with at least one embodiment;

FIG. 278 is another top partial cross-sectional view of the adjunct, anvil and end effector of FIG. 277 with the knife in an intermediate position in accordance with at least one embodiment;

FIG. 279 is another top partial cross-sectional view of the adjunct, anvil, and end effector of FIGS. 277 and 278 showing an adjunct attachment assembly of the adjunct partially torn from the adjunct in accordance with at least one embodiment;

FIG. 280 is a perspective view of a staple cartridge for use with a surgical stapling instrument;

FIG. 281 is a partial plan view of a deck surface comprising trace retention features of the staple cartridge of FIG. 280;

FIG. 282 is a partial plan view of a flexible circuit;

FIG. 283 is a partial plan view of the deck surface of FIG. 281 comprising the flexible circuit of FIG. 282 secured there against;

FIG. 284 is a partial cross-sectional view of the deck surface and the flexible circuit of FIG. 283;

FIG. 285 is a partial perspective view of a deck surface comprising a trace retention feature in a first configuration;

FIG. 286 is a partial perspective view of the deck surface of FIG. 285 with the trace retention feature in a second configuration;

FIG. 287 is a partial plan view of a staple cartridge comprising electrical traces printed directly on a deck surface thereof;

FIG. 288 is a partial perspective view of the staple cartridge of FIG. 287;

FIG. 289 is a perspective view of a staple cartridge for use with a surgical stapling instrument in accordance with at least one embodiment;

FIG. 290 is a partial perspective view of the staple cartridge of FIG. 289 comprising a staple cavity and staple retention features configured to maintain alignment of a staple within the staple cavity;

FIG. 291 is a plan view of the staple cavity of FIG. 290 with a first staple stored therein;

FIG. 292 is a plan view of a staple cavity lacking the staple retention features of FIG. 290 with the first staple of FIG. 291 stored therein;

FIG. 293 is a plan view of the staple cavity of FIG. 290 with a second staple stored therein;

FIG. 294 is a plan view of the staple cavity of FIG. 292 with the second staple of FIG. 293 stored therein;

FIG. 295 is a partial plan view of a staple cartridge for use with a surgical stapling instrument in accordance with at least one embodiment, wherein the staple cartridge comprises projections extending from a deck surface;

FIG. 296 is a partial plan view of the staple cartridge of FIG. 295;

FIG. 297 is a partial elevational view of the staple cartridge of FIG. 295;

FIG. 298 illustrates a firing drive of a surgical stapling instrument in an unarticulated and unfired configuration in accordance with at least one embodiment;

FIG. 299 illustrates the firing drive of FIG. 298 in an articulated and unfired configuration;

FIG. 300 illustrates the firing drive of FIG. 298 in an articulated and fired configuration;

FIG. 301 is a partial elevational view of a surgical stapling instrument including a closing drive in an open configuration and a firing drive in an unfired configuration in accordance with at least one embodiment;

FIG. 302 illustrates the closing drive of FIG. 301 in a closed configuration and the firing drive of FIG. 301 in an unfired configuration;

FIG. 303 is a partial elevational view of a firing drive of a surgical stapling instrument in accordance with at least one embodiment;

FIG. 304 illustrates the firing drive of FIG. 303 in a fired configuration;

FIG. 305 is a partial elevational view of a firing drive of a surgical stapling instrument in accordance with at least one embodiment; and

FIG. 306 is an elevational view of a end effector of a surgical stapling instrument in accordance with at least one embodiment.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

A surgical stapling system can comprise a shaft and an end effector extending from the shaft. The end effector comprises a first jaw and a second jaw. The first jaw comprises a staple cartridge. The staple cartridge is insertable into and removable from the first jaw; however, other embodiments are envisioned in which a staple cartridge is not removable from, or at least readily replaceable from, the first jaw. The second jaw comprises an anvil configured to deform staples ejected from the staple cartridge. The second jaw is pivotable relative to the first jaw about a closure axis; however, other embodiments are envisioned in which the first jaw is pivotable relative to the second jaw. The surgical stapling system further comprises an articulation joint configured to permit the end effector to be rotated, or articulated, relative to the shaft. The end effector is rotatable about an articulation axis extending through the articulation joint. Other embodiments are envisioned which do not include an articulation joint.

The staple cartridge comprises a cartridge body. The cartridge body includes a proximal end, a distal end, and a deck extending between the proximal end and the distal end. In use, the staple cartridge is positioned on a first side of the tissue to be stapled and the anvil is positioned on a second side of the tissue. The anvil is moved toward the staple cartridge to compress and clamp the tissue against the deck. Thereafter, staples removably stored in the cartridge body can be deployed into the tissue. The cartridge body includes staple cavities defined therein wherein staples are removably stored in the staple cavities. The staple cavities are arranged in six longitudinal rows. Three rows of staple cavities are positioned on a first side of a longitudinal slot and three rows of staple cavities are positioned on a second side of the longitudinal slot. Other arrangements of staple cavities and staples may be possible.

The staples are supported by staple drivers in the cartridge body. The drivers are movable between a first, or unfired position, and a second, or fired, position to eject the staples from the staple cavities. The drivers are retained in the cartridge body by a retainer which extends around the bottom of the cartridge body and includes resilient members configured to grip the cartridge body and hold the retainer to the cartridge body. The drivers are movable between their unfired positions and their fired positions by a sled. The sled is movable between a proximal position adjacent the proximal end and a distal position adjacent the distal end. The sled comprises a plurality of ramped surfaces configured to slide under the drivers and lift the drivers, and the staples supported thereon, toward the anvil.

Further to the above, the sled is moved distally by a firing driver. The firing driver is configured to contact the sled and push the sled toward the distal end. The longitudinal slot defined in the cartridge body is configured to receive the firing driver. The anvil also includes a slot configured to receive the firing driver. The firing driver further comprises a first cam which engages the first jaw and a second cam which engages the second jaw. As the firing driver is advanced distally, the first cam and the second cam can control the distance, or tissue gap, between the deck of the staple cartridge and the anvil. The firing driver also comprises a knife configured to incise the tissue captured intermediate the staple cartridge and the anvil. It is desirable for the knife to be positioned at least partially proximal to the ramped surfaces such that the staples are ejected ahead of the knife.

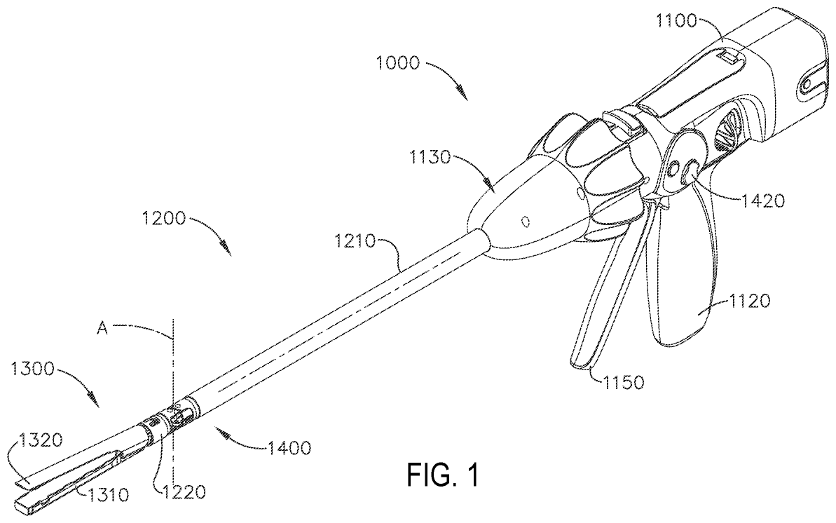

A surgical stapling instrument 1000 is illustrated in FIG. 1. The stapling instrument 1000 comprises a handle 1100, an elongate shaft 1200 extending from the handle 1100, and an end effector 1300 including a cartridge jaw 1310 and an anvil jaw 1320. The handle 1100 comprises a housing 1110 including a grip 1120 that is sized and configured to be held by a clinician. The handle 1100 further comprises triggers that are actuatable by the clinician to operate the stapling instrument 1000 as described further below. In various other embodiments, the stapling instrument 1000 comprises a housing assembly configured to be attached to a robotic surgical instrument system instead of the handle 1100. In at least one such embodiment, the housing assembly comprises rotatable inputs that are operably coupled with motor-driven outputs of the robotic surgical instrument system when the housing assembly is attached to the robotic surgical instrument system. The robotic surgical instrument system comprises a control station including triggers that are actuatable by a clinician to operate the surgical instrument 1000. The entire disclosure of U.S. Pat. No. 9,072,535, entitled SURGICAL STAPLING INSTRUMENT WITH ROTATABLE DEPLOYABLE ARRANGEMENTS, which issued on Jul. 7, 2015, is incorporated by reference herein.

The elongate shaft 1200 is rotatable relative to handle 1100 about a longitudinal axis L. When the elongate shaft 1200 is rotated relative to the handle 1100, the end effector 1300 rotates with the elongate shaft 1200. The handle 1100 comprises a rotation actuator 1130 mounted to the elongate shaft 1200 that is rotatable relative to the handle housing 1110 by the clinician to rotate the shaft 1200 about the longitudinal axis L. In various embodiments, the stapling instrument 1000 comprises a motor-driven system that is operable to rotate the elongate shaft 1200. In at least one such embodiment, the motor-driven system comprises an electric motor mounted in the handle 1100 that includes an output gear meshingly engaged with a ring of gear teeth defined on the elongate shaft 1200. The motor-driven system further comprises a trigger, such as a switch, for example, accessible by the clinician operating the stapling instrument 1000. In at least one such embodiment, the actuator is positioned on the handle 1100.

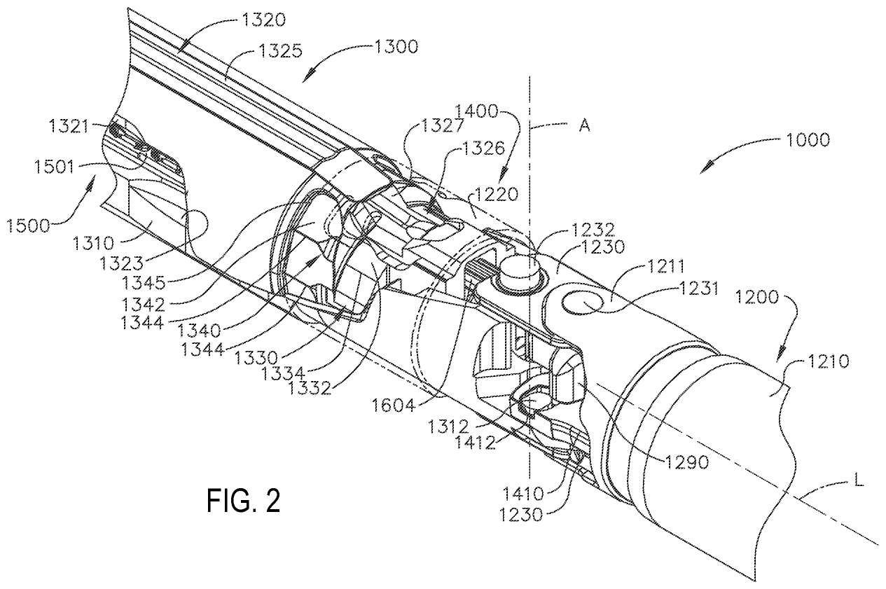

The end effector 1300 is rotatable relative to the shaft 1200 about an articulation joint 1400. The articulation joint 1400 defines an articulation axis A about which the end effector 1300 is articulated relative to the shaft 1200. The articulation joint 1400 also defines a plane within which the end effector 1300 is articulated relative to the shaft 1200. The shaft 1200 comprises a projection extending from a frame 1290 of the shaft 1200 that is closely received within an aperture defined in the cartridge jaw 1310 where the projection and the aperture define a pivot joint about which the end effector 1300 is articulated. The stapling instrument 1000 further comprises an articulation drive system. Referring to FIGS. 1 and 2, the articulation drive system comprises an articulation driver 1410, an electric motor operable to drive the articulation driver 1410 longitudinally, and a motor control circuit including a trigger 1420, such as a switch, for example, accessible by the clinician operating the stapling instrument 1000. The trigger 1420 is positioned on the handle 1100, but could be positioned in any suitable location on the stapling instrument 1000. The cartridge jaw 1310 comprises an articulation drive pin 1312 and the articulation driver 1410 comprises a distal end 1412 comprising an aperture defined therein within which the articulation drive pin 1312 is positioned. When the articulation driver 1410 is translated distally by the articulation drive system, the end effector 1300 rotates in a first direction and, when the articulation driver 1410 is translated proximally by the articulation drive system, the end effector 1300 rotates in a second, or opposite, direction. In various other embodiments, the articulation joint 1400 defines more than one articulation axis, such as two articulation axes, for example, about which the end effector 1300 can be rotated relative to the shaft 1200. In at least one embodiment, the articulation joint 1400 comprises a flexible articulation region. The entire disclosure of U.S. Pat. No. 9,629,629, entitled CONTROL SYSTEMS FOR SURGICAL INSTRUMENTS, which issued on Apr. 25, 2017, is incorporated by reference herein.

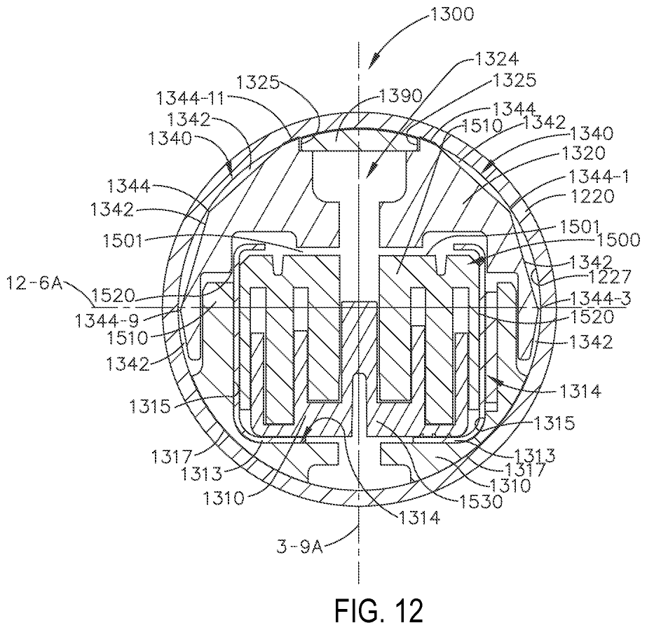

Further to the above, the end effector 1300 comprises a cartridge jaw 1310 and an anvil jaw 1320. Referring to FIG. 12, the cartridge jaw 1310 comprises a channel 1314 configured to receive a replaceable staple cartridge 1500. The channel 1314 comprises a bottom 1313 and lateral sidewalls 1315 extending upwardly from the bottom 1313. The staple cartridge 1500 comprises a cartridge body 1510 and a pan 1520 attached to the cartridge body 1510. That said, other embodiments are envisioned in which the staple cartridge 1500 does not comprise a pan. The cartridge body 1510 comprises a deck 1501 configured to support patient tissue and longitudinal rows of staple cavities defined in the deck 1501. The staple cartridge 1500 further comprises staples stored in the staple cavities and staple drivers movable within the staple cavities to eject the staples from the staple cavities during a staple firing stroke. The staple cartridge 1500 further comprises a sled 1530 (FIG. 12) movable from a proximal position to a distal position during the staple firing stoke to lift the staple drivers and eject the staples during the staple firing stroke. The staple cartridge 1500 and/or the cartridge jaw 1310 comprise features which releasably lock the staple cartridge 1500 in the cartridge jaw 1310 such that the staple cartridge 1500 is secured in position in the cartridge jaw 1310 but can be replaced during a surgical procedure. In various other embodiments, the staple cartridge 1500 is secured in the cartridge jaw 1310 in a manner that does not allow the staple cartridge 1500 to be released from the cartridge jaw 1310 and replaced during a surgical procedure. The entire disclosure of U.S. Pat. No. 11,045,191, entitled METHOD FOR OPERATING A SURGICAL STAPLING SYSTEM, which issued on Jun. 29, 2021 is incorporated by reference herein.

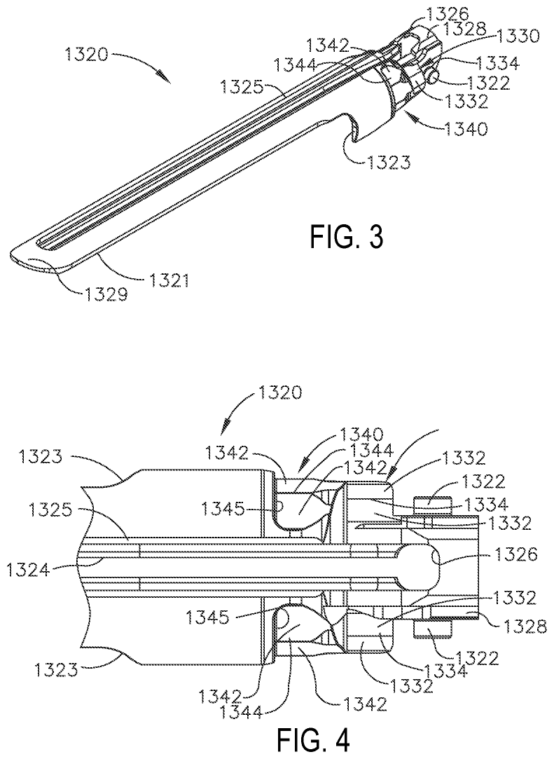

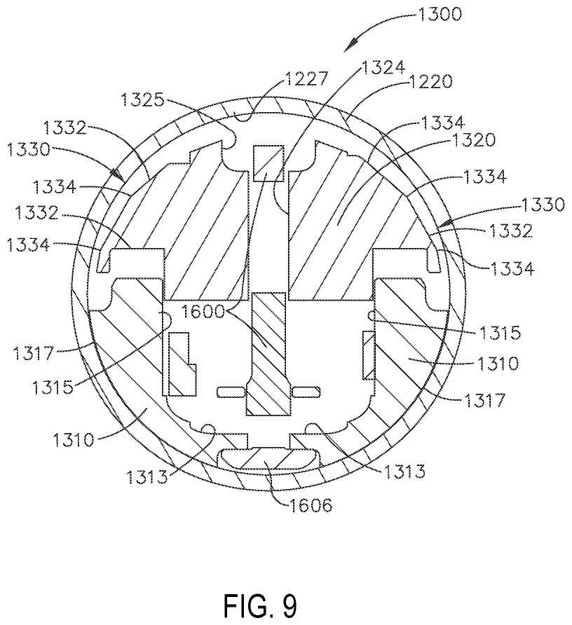

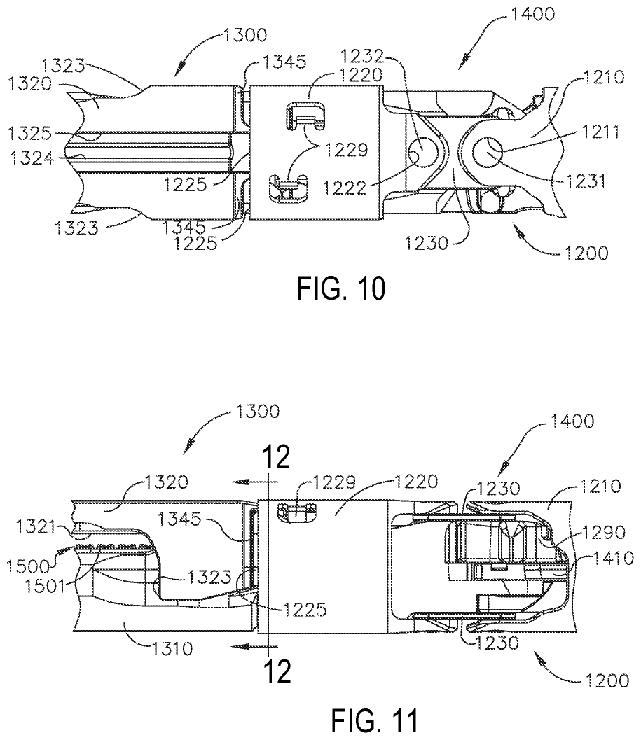

Further to the above, the anvil jaw 1320 is rotatably connected to the cartridge jaw 1310. Referring to FIGS. 3 and 4, the anvil jaw 1320 comprises pivot pins 1322 extending laterally therefrom. The pivot pins 1322 are rotatably secured in apertures defined in the cartridge jaw 1310 such that the anvil jaw 1320 rotates about a pivot axis PA. The anvil jaw 1320 further comprises a tissue compression surface 1321 that is brought into opposition with the deck 1501 of the staple cartridge 1500 when the anvil jaw 1320 is rotated between a fully open position (FIG. 8) and a fully closed position (FIG. 11). The anvil jaw 1320 further comprises staple forming pockets arranged in longitudinal rows that are registered with the staple cavities defined in the staple cartridge 1500 when the anvil jaw 1320 is in its fully-closed position. The anvil jaw 1320 further comprises tissue stops 1323 that extend downwardly toward the cartridge jaw 1310 and are positioned inwardly with respect to the lateral sides of the cartridge jaw 1310. In other embodiments, the tissue stops 1323 of the anvil jaw 1320 are positioned outwardly with respect to the lateral sides of the cartridge jaw 1310. The tissue stops 1323 inhibit the migration of tissue proximally into the end effector 1300 past the tissue stops 1323 which can reduce the possibility of the tissue coming into contact with a tissue cutting edge of a firing bar 1600 parked in a proximal unfired position (FIG. 2). In other embodiments, the staple cartridge comprises the tissue cutting edge; however, the tissue stops 1323 would still provide the same benefit. The firing bar 1600 comprises an elongate portion 1604 extending through the articulation joint 1400 and a cam that is at least partially positioned in a window 1326 defined through the top portion of the anvil jaw 1320 when the firing bar 1600 is in its proximal unfired position. When the firing bar 1600 is advanced distally during the staple firing stroke, the firing bar 1600 travels within a longitudinal slot 1324 defined in the top portion of the anvil jaw 1320. The anvil jaw 1320 further comprises a longitudinal recess 1325 that defines longitudinal shoulders on opposite sides of the longitudinal slot 1324 which are engaged by the cam to hold the anvil jaw 1320 in position during the staple firing stroke. Referring to FIG. 12, the anvil jaw 1320 further comprises a cap 1390 affixed thereto that extends over the longitudinal slot 1324 and the longitudinal recess 1325. Referring to FIG. 9, the firing bar 1600 further comprises a cam 1606 that engages the cartridge jaw 1310 during the staple firing stroke and co-operates with the anvil-engaging cam to hold the anvil jaw 1320 and the cartridge jaw 1310 in position relative to one another.

Further to the above, the anvil jaw 1320 is movable from a fully-open position (FIG. 8) to a fully-closed position (FIG. 11) to clamp tissue between the anvil jaw 1320 and the staple cartridge 1500. The stapling instrument 1000 further comprises a jaw closure system configured to move the anvil jaw 1320 into its fully-closed position. The jaw closure system comprises a trigger 1150 rotatably coupled to the handle 1100, a closure carriage positioned in the handle 1100 translatable distally by the trigger 1150, and a closure tube 1210 supported by the closure carriage such that the closure tube 1210 translates with the closure carriage. The trigger 1150 comprises a rotatable lever movable from an unactuated position to an actuated position to transmit a force applied to the trigger 1150 by a clinician to the closure tube 1210. When the trigger 1150 is pulled toward the grip 1120 by the clinician, the trigger 1150 pushes the closure carriage and the closure tube 1210 distally. The closure tube 1210 comprises a longitudinal aperture defined therein through which the shaft frame 1290 and the articulation driver 1410 extend. The jaw closure system further comprises a distal closure tube 1220 rotatably connected to the closure tube 1210 by two links 1230. Each link 1230 comprises a proximal projection 1231 closely received within an aperture 1211 defined in the closure tube 1210 and, in addition, a distal projection 1232 closely received within an aperture 1222 defined in the distal closure tube 1220. When the closure tube 1210 is driven distally by the jaw closure system, as described above, the closure tube 1210 pushes the links 1230 and the distal closure tube 1220 distally such that the closure tube 1210, the links 1230, and the distal closure tube 1220 translate relative to the articulation joint 1400. The links 1230 are sized and configured to permit the jaw closure drive to transmit a closing force across the articulation joint 1400 even when the end effector 1300 is in an articulated position.

In various alternative embodiments, the jaw closure system comprises an electric motor configured to drive the closure tube 1210 distally through the closure stroke when the electric motor is operated in a first direction and retract the closure tube 1210 proximally when the electric motor is operated in a second, or opposite, direction. In at least one such embodiment, the jaw closure system comprises a motor control circuit configured to control the operation of the electric motor. The motor control circuit comprises a processor and a trigger that is actuatable by the clinician to operate the electric motor.

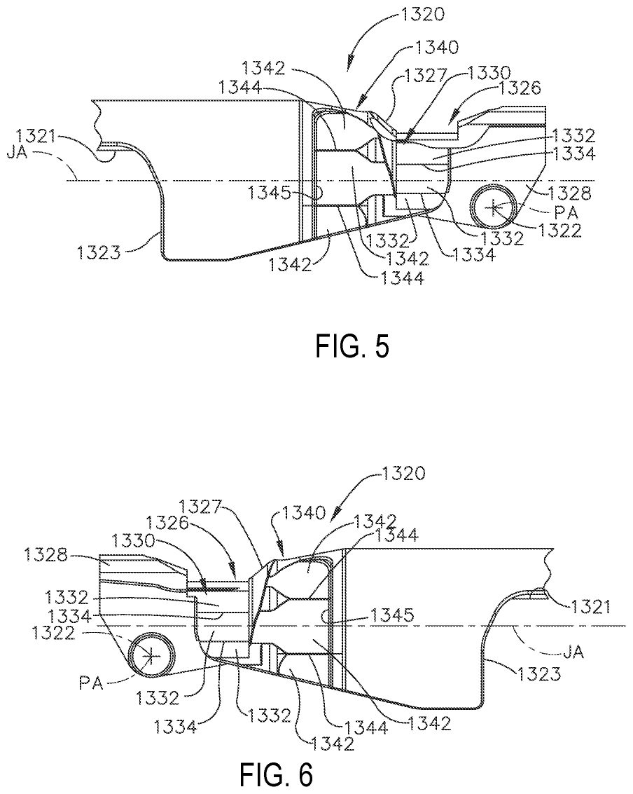

Referring to FIGS. 5 and 6, the anvil jaw 1320 comprises a proximal end 1328, a distal end 1329, and a jaw axis JA extending between a proximal end 1328 and a distal end 1329 of the anvil jaw 1320. The anvil jaw 1320 further comprises closure ramps 1327 defined thereon positioned distally with respect to the pivot pins 1322. When the distal closure tube 1220 is advanced distally by the closure drive during a closure stroke, further to the above, the distal closure tube 1220 contacts the closure ramps 1327 and moves the anvil jaw 1320 from its fully-open position into a partially-closed position. This partially-closed position comprises a state of closure intermediate the fully-open position and a fully-closed position. As the anvil jaw 1320 is advanced further distally during the closure stroke, the distal closure tube 1220 comes into contact with contact ridges 1344 defined on the anvil jaw 1320 to move the anvil jaw 1320 into a fully-closed position. More specifically, an inner surface 1227 of the distal closure tube 1220 contacts the contact ridges 1344 and slides along the contact ridges 1344 as the distal closure tube 1220 is moved to the end of the closure stroke. Referring primarily to FIG. 12, the inner surface 1227 of the distal closure tube 1220 is in contact with an outside surface 1317 of the cartridge jaw 1310 when the distal closure tube 1220 is in contact with the contact ridges 1344. The distal closure tube 1220 rotates the anvil jaw 1320 toward the cartridge jaw 1310 when the distal closure tube 1220 is moved distally during the closure stroke and holds the anvil jaw 1320 in position relative to the cartridge jaw 1310.

Referring primarily to FIGS. 4 and 12, further to the above, the contact ridges 1344 are arranged in an array extending about a periphery 1340 of the anvil jaw 1320. The top of the anvil jaw 1320, i.e., the portion of the anvil jaw 1320 facing away from the cartridge jaw 1310, has a generally arcuate shape defining an outer circumference and the contact ridges 1344 define part of the outer circumference. The contact ridges 1344 extend longitudinally and are parallel to one another and parallel to the jaw axis JA. In various other embodiments, the contact ridges 1344 are substantially parallel to one another and are within 10 degrees of being parallel to the jaw axis JA. The periphery of the anvil jaw 1320 further comprises surfaces 1342 positioned intermediate the contact ridges 1344. The surfaces 1342 are flat; however, the surfaces 1342 can comprise any suitable shape, such as an arcuate shape, for example. Each surface 1342 extends between and connects adjacent contact ridges 1344. Referring primarily to FIG. 11, the distal closure tube 1220 is in contact with the contact ridges 1344, but not the surfaces 1342. Instead, clearance gaps are present between the surfaces 1342 and the inner surface 1227 of the distal closure tube 1220. As a result, the contact ridges 1344 comprise discrete control points between the distal closure tube 1220 and the anvil jaw 1320. A first set, or segment, of contact ridges 1344 are positioned on a first side of the jaw axis JA which comprise a first set of control points and a second set, or segment, of contact ridges 1344 are positioned on a second, or opposite, side of the jaw axis JA which comprise a second set of control points. Owing to the control points between the distal closure tube 1220 and the anvil jaw 1320, and the contact between the distal closure tube 1220 and the cartridge jaw 1310, the distal closure tube 1220 holds the anvil jaw 1320 in position relative to the cartridge jaw 1310. The first set of contact ridges 1344 and the second set of contact ridges 1344 are separated by a gap, or break, in the array of contact ridges 1344. Alternative embodiments are envisioned in which the array of contact ridges 1344 is continuous about the outer periphery of the anvil jaw 1320. The entire disclosure of U.S. Pat. No. 11,207,065, entitled METHOD FOR FABRICATING SURGICAL STAPLER ANVILS, which issued on Dec. 28, 2021 is incorporated by reference herein.

To insert the end effector 1300 into a patient, further to the above, the anvil jaw 1320 can be moved into a fully-closed position without patient tissue positioned between the anvil jaw 1320 and the staple cartridge 1500. For instance, the anvil jaw 1320 can be moved into a fully-closed so as to insert the end effector 1300 into a patient through a trocar, or cannula, for example. In such instances, the closure drive is configured to lock the anvil jaw 1320 in its fully-closed position. In at least one embodiment, the handle 1100 comprises a releasable lock configured to hold the closure trigger 1150 in a fully-actuated position to hold the anvil jaw 1320 in its fully-closed position. To open the anvil jaw 1320, the lock is releasable by the clinician which allows the closure trigger 1150 to return to its unactuated position via a return spring and/or the clinician moving the closure trigger 1150 back into its unactuated position. As the closure trigger 1150 is being returned back into its unactuated position, the closure trigger 1150 pulls the closure carriage, the closure tube 1210, the closure links 1230, and the distal closure tube 1220 proximally to disengage the distal closure tube 1220 from the contact ridges 1344 and the closure ramps 1327. The distal closure tube 1220 further comprises tabs 1229 extending radially inwardly toward the anvil jaw 1320. When the distal closure tube 1220 is being pulled proximally, the tabs 1229 are configured to contact the anvil jaw 1320 and pull, or positively open, the anvil jaw 1320. In addition to or in lieu of the positive jaw opening tabs 1229, alternative embodiments are envisioned in which the end effector 1300 comprises one or more biasing members, such as springs, for example, configured to bias the anvil jaw 1320 into, or at least toward, its fully-open position.

Further to the above, the distal end 1329 of the anvil jaw 1320 is in contact with the staple cartridge 1500 when the anvil jaw 1320 is in its fully-closed position and the space between the anvil jaw 1320 and the staple cartridge 1500 is empty. In such instances, the tissue compression surface 1321 of the anvil jaw 1320 extends within a plane extending downwardly toward the staple cartridge 1500. In various alternative embodiments, the distal end 1329 of the anvil jaw 1320 is not in contact with the staple cartridge 1500 when the anvil jaw 1320 is in its fully-closed position and the space between the anvil jaw 1320 and the staple cartridge 1500 is empty. In many instances, however, patient tissue is captured between the anvil jaw 1320 and the staple cartridge 1500 when the anvil jaw 1320 is moved into its fully-closed position. In such instances, the orientation of the anvil jaw 1320 relative to the staple cartridge 1500 depends on thickness and/or density of the tissue, for example, captured between the anvil jaw 1320 and the staple cartridge 1500. Thus, the anvil jaw 1320 may be in one orientation when the captured patient is thick and another orientation when the captured patient tissue is thin; however, both orientations, along with other orientations of the anvil jaw 1320, comprise fully-closed positions of the anvil jaw 1320.

At the end of the closure stroke, further to the above, a distal end 1225 of the distal closure tube 1220 is adjacent a ledge 1345 defined at the distal end of the contact ridges 1344. The distance between the distal end 1225 and the ledge 1345 at the end of the closure stroke depends on, among other things, the thickness and/or density of the patient tissue captured between the anvil jaw 1320 and the staple cartridge 1500. For instance, the distal end 1225 of the distal closure tube 1220 is positioned further away from the ledge 1345 when the captured patient tissue is thick as compared to when the captured patient tissue is thin. In various other embodiments, the distal end 1225 can come into contact with the ledge 1345 at the end of the closure stroke which can push the anvil jaw 1320 into its fully-closed position. In various instances, the clinician can re-open the anvil jaw 1320 by reversing the operation of the electric motor via the control circuit actuator and, if desired, re-position the jaws 1310 and 1320 relative to the patient tissue.

When the anvil jaw 1320 is in a fully-closed position, further to the above, the staple firing system can be actuated to fire the staples from the staple cartridge 1500. The staple firing system comprises an electric motor and a control circuit configured to control the electric motor. The control circuit of the staple firing system comprises a processor and a trigger that, when actuated by a clinician, controls the operation of the electric motor to move the firing bar 1600 distally to perform a staple firing stroke. The firing bar 1600 comprises a distally-presented tissue cutting edge and, during the staple firing stroke, the patient tissue captured between the staple cartridge 1500 and the anvil jaw 1320 is stapled and incised. After the staple firing stroke has been performed, or at least partially performed, the electric motor can be operated in an opposite direction to retract the firing bar 1600. Once the firing bar 1600 has been sufficiently retracted, the anvil jaw 1320 can be re-opened to unclamp the stapled and incised patient tissue.

As described above, the surgical instrument 1000 is a stapling instrument. That said, the teachings and embodiments disclosed herein can be adapted to any suitable surgical instrument. In at least one embodiment, a suitable surgical instrument comprises an electrocautery instrument that applies electrical energy to patient tissue, for example.

Further to the above, the anvil jaw 1320 comprises an array of ridges 1334. The ridges 1334 are positioned proximally with respect to the contact ridges 1344 and the array of ridges 1334 extends about a periphery 1330 of the anvil jaw 1320 that is proximal to the periphery including the contact ridges 1344. The periphery including the ridges 1334 has a generally arcuate shape defining an outer circumference and the ridges 1334 define part of the outer circumference. The ridges 1334 extend longitudinally and are parallel to one another and parallel to the jaw axis JA. In various other embodiments, the ridges 1334 are substantially parallel to one another and are within 10 degrees of being parallel to the jaw axis JA. The periphery of the anvil jaw 1320 further comprises relief surfaces 1332 positioned intermediate the ridges 1334. The relief surfaces 1332 are flat; however, the relief surfaces 1332 can comprise any suitable shape, such as an arcuate shape, for example. Each relief surface 1332 extends between and connects adjacent ridges 1334.

Further to the above, the ridges 1334 are not in contact with the distal closure tube 1220 when the anvil jaw 1320 is in its fully-closed position or any of its partially-closed positions. Referring primarily to FIG. 9, however, the ridges 1334 can come into contact with the anvil jaw 1320 when the anvil jaw 1320 is in its fully-open position. That said, clearance gaps are present between the relief surfaces 1332 and the distal closure tube 1220 through out the entire range of motion of the anvil jaw 1320. A first set, or segment, of ridges 1334 are positioned on a first side of the jaw axis JA which comprise a first set of control points and a second set, or segment, of ridges 1334 are positioned on a second, or opposite, side of the jaw axis JA which comprise a second set of control points. Owing to the control points between the distal closure tube 1220 and the anvil jaw 1320 when the anvil jaw 1320 is in its fully-open position, the distal closure tube 1220 can limit the upward, or opening, rotation of the anvil jaw 1320. The first set of ridges 1334 and the second set of ridges 1334 are separated by a gap, or break, in the array of ridges 1334 but could comprise a continuous array without a gap, or break, therein. The above being said, embodiments are envisioned in which the distal closure tube 1220 engages the contact ridges 1344 to close the anvil jaw 1320 but does not engage the ridges 1334 of the anvil jaw 1320.

Referring to FIGS. 4-6, the closure ramps 1327 extend between and connect the array of ridges 1334 and relief surfaces 1332 and the array of contact ridges 1344 and surfaces 1342. That said, embodiments are envisioned in which the closure ramps 1327 are connected to the array of contact ridges 1344 and surfaces 1342 but not the array of ridges 1334 and relief surfaces 1332. Various other embodiments are envisioned without the closure ramps 1327. In at least one such embodiment, a longitudinal gap is present between the array of ridges 1334 and relief surfaces 1332 and the array of contact ridges 1344 and surfaces 1342.

Referring to FIGS. 4-6, the ridges 1334 are not aligned with the contact ridges 1344. The contact ridges 1344 extend about a first circumferential periphery of the anvil jaw 1320 and the ridges 1334 extend about a second circumferential periphery of the anvil jaw 1320 where the second circumferential periphery is closer to the longitudinal jaw axis JA than the first circumferential periphery. The ridges 1334 are closer to the longitudinal jaw axis JA than the contact ridges 1344 and, as a result, the ridges 1334 are longitudinally unaligned with the contact ridges 1344. The first circumferential periphery and the second circumferential periphery are centered about the longitudinal jaw axis JA; however, alternative embodiments are envisioned where the first circumferential periphery and the second circumferential periphery extend about, but are not centered about, the longitudinal jaw axis JA. In addition, the ridges 1334 are declocked, or unregistered, with the contact ridges 1344 about the longitudinal jaw axis JA. When viewed distally end-on along the longitudinal jaw axis JA, the ridges 1334 are aligned with the surfaces 1342 and, similarly, the contact ridges 1344 are aligned with the relief surfaces 1332 and, as a result, the ridges 1334 are longitudinally unaligned with the contact ridges 1344. As a result of this arrangement, the relief surfaces 1332 create clearance gaps between the distal closure tube 1220 and the anvil jaw 1320 that are aligned with the contact ridges 1344. Such clearance gaps permit the distal closure tube 1220 to engage the contact ridges 1344 and control the anvil jaw 1320 without interference from the periphery including the ridges 1334.

As discussed above, the jaw closure system of the stapling instrument 1000 comprises, among other things, a closure tube 1210 and a distal closure tube 1220 rotatably connected to the closure tube 1210. As also discussed above, the distal closure tube 1220 engages the cartridge jaw 1310 and the anvil jaw 1320 during the closure stroke to position the anvil jaw 1320 relative to the cartridge jaw 1310. The distal closure tube 1220 defines a perimeter that entirely surrounds the cartridge jaw 1310 and the anvil jaw 1320; however, embodiments are envisioned in which the distal closure tube 1220 only partially surrounds the cartridge jaw 1310 and/or the anvil jaw 1320. In at least one such embodiment, the distal closure tube 1220 comprises one or more apertures defined therein. In various embodiments, the distal closure tube 1220 comprises a first cantilever that engages the cartridge jaw 1310 and a second cantilever that engages the anvil jaw 1320 where the distal ends of the first cantilever and the second cantilever are unconnected. In various embodiments, the jaw closure drive comprises any suitable closure driver that engages the anvil jaw 1320 and moves the anvil jaw 1320 into a closed position. In at least one embodiment, the closure driver comprises a closure bar that engages an outer perimeter of the cartridge jaw 1310 and/or anvil jaw 1320. In various alternative embodiments, one or both of the ridges 1334 and 1344 are not defined on the outer perimeter of the anvil jaw 1320 and are instead defined in an internal cavity defined in the anvil jaw 1320. In such embodiments, the closure driver, such as a bar, for example, enters into the internal cavity defined in the anvil jaw 1320 to engage the ridges 1334 and/or 1344, and/or any other suitable contact surfaces.

As described above, the anvil jaw 1320 is rotatable relative to the cartridge jaw 1310. Alternative embodiments are envisioned in which the cartridge jaw 1310 is rotatable relative to the anvil jaw 1320. In at least one such embodiment, the cartridge jaw 1310 comprises an array of ridges 1334 and relief surfaces 1332 and an array of contact ridges 1344 and surfaces 1342. Similar to the above, the distal closure tube 1320 is configured to engage the contact ridges 1344 on the cartridge jaw 1310 and move the cartridge jaw 1310 into a closed position opposite the anvil jaw 1320. In various alternative embodiments, the cartridge jaw 1310 and the anvil jaw 1320 are both movable relative to one another. In at least one such embodiment, the cartridge jaw 1310 comprises an array of ridges 1334 and relief surfaces 1332 and an array of contact ridges 1344 and surfaces 1342 and, also, the anvil jaw 1320 comprises an array of ridges 1334 and relief surfaces 1332 and an array of contact ridges 1344 and surfaces 1342. Similar to the above, the distal closure tube 1320 is configured to engage the contact ridges 1344 on the cartridge jaw 1310 and the anvil jaw 1320 to move the cartridge jaw 1310 and the anvil jaw 1320 into closed positions.

Referring again to FIGS. 4-6, adjacent surfaces 1342 extend at an angle to one another. Each contact ridge 1344 is defined as an edge extending between adjacent surfaces 1342. In at least one embodiment, the surfaces 1342 are ground flat and the ridges 1344 comprise sharp edges defined between the surfaces 1342. Such embodiments provide a discrete line, or discrete lines, in which the distal closure tube 1220 contacts. In other embodiments, the contact ridges 1344 comprise radiused, or rounded, edges extending between adjacent surfaces 1342. Such embodiments can reduce galling and/or sticking between the distal closure tube 1220 and the anvil jaw 1320. Similarly, adjacent relief surfaces 1332 extend at an angle to one another. Each contact ridge 1334 is defined as an edge extending between adjacent relief surfaces 1332. In at least one embodiment, the relief surfaces 1332 are ground flat and the ridges 1334 comprise sharp edges defined between the relief surfaces 1332. In other embodiments, the ridges 1334 comprise radiused, or rounded, edges extending between adjacent relief surfaces 1332.

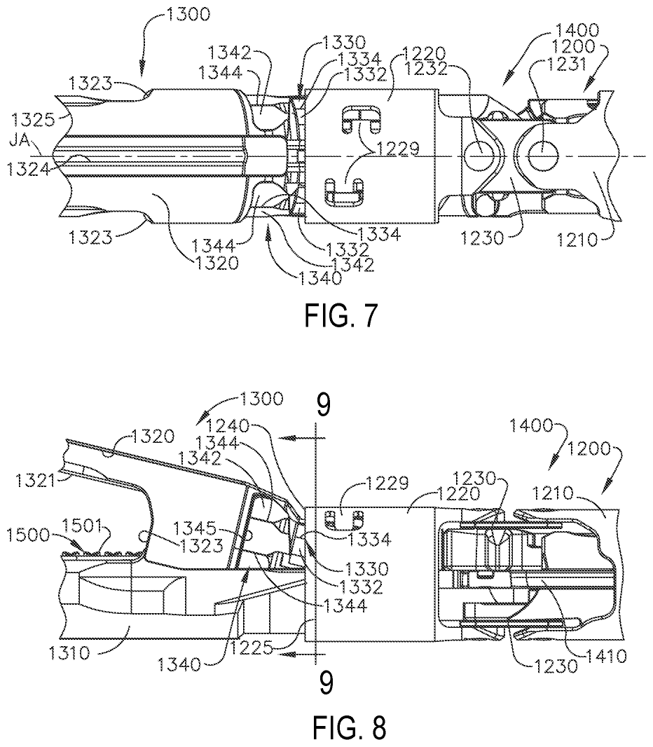

Further to the above, the relief surfaces 1332 allow the anvil jaw 1320 to move between its fully-open and fully-closed positions without the anvil jaw 1320 getting jammed during its open and closing motions. As discussed above, referring again to FIG. 7, the jaw tabs 1229 of the distal closure tube 1220 engage the anvil jaw 1320 as the distal closure tube 1220 is pulled proximally to rotate the anvil jaw 1320 into its fully-open position. As illustrated in FIG. 7, one of the jaw opening tabs 1229 is positioned distally with respect to the other. As also illustrated in FIG. 7, the distal jaw opening tab 1229 is positioned on a first lateral side of the distal closure tube 1220 and the proximal jaw opening tab 1229 is positioned on a second lateral side of the distal closure tube 1220. Stated another way, the jaw opening tabs 1229 are staggered longitudinally and off-center with respect to the jaw axis JA. Owing to this arrangement, the jaw opening tabs 1229 contact the anvil jaw 1320 sequentially as the distal closure tube 1220 is retracted. As the distal closure tube 1220 is retracted during an initial jaw opening motion, the distal jaw tab 1229 contacts the anvil jaw 1320 without the proximal jaw tab 1229 being in contact with the anvil jaw 1320. In such instances, the distal jaw tab 1229 pulls the anvil jaw 1320 into a partially open position by itself. As the distal closure tube 1220 is retracted further, the proximal jaw tab 1229 comes into contact with the anvil jaw 1320 and pulls the anvil jaw 1320 into its fully-open position during a second opening motion. In various instances, the initial jaw opening motion and the second opening motion comprise one continuous motion while, in other instances, a pause is present between the initial jaw opening motion and the second opening motion. The distal jaw tab 1229 disengages from the anvil jaw 1320 before or when the proximal jaw tab 1229 comes into contact with the anvil jaw 1320. In other embodiments, the distal jaw tab 1229 and the proximal jaw tab 1229 are simultaneously in contact with the anvil jaw 1320 for at least a portion of the second opening motion. In either event, the sequential co-operation of the two jaw opening tabs 1229 allows the anvil jaw 1320 to be opened wider than could be done with a single jaw opening tab. A wider open position of the anvil jaw 1320 creates a larger space, or jaw aperture, between the anvil jaw 1320 and the cartridge jaw 1310 that facilitates the positioning of the patient tissue between the jaws 1310 and 1320. Further to the above, the longitudinally staggered and laterally offset arrangement of the jaw opening tabs 1229 allow for this advantage to be obtained; however, other arrangements could be used. Concurrent with the wider fully-open position of the anvil jaw 1320 provided by the arrangement of the jaw opening tabs 1229, the relief surfaces 1332 of the anvil jaw 1320 provide sufficient clearance for the anvil jaw 1320 to move into such a wider fully-open position. The entire disclosures of U.S. Pat. No. 10,182,818, entitled SURGICAL END EFFECTORS WITH POSITIVE JAW OPENING ARRANGEMENTS, which issued on Jan. 22, 2019, U.S. Pat. No. 10,758,232, entitled SURGICAL INSTRUMENT WITH POSITIVE JAW OPENING FEATURES, which issued on Sep. 1, 2020, U.S. Pat. No. 11,207,065, entitled METHOD FOR FABRICATING SURGICAL STAPLER ANVILS, which issued on Dec. 28, 2021, and U.S. Patent Application Publication No. 2018/0168575, entitled SURGICAL STAPLING SYSTEMS, which published on Jun. 21, 2018, are incorporated by reference herein.

In various alternative embodiments, further to the above, the proximal jaw tab 1229 contacts the anvil jaw 1320 without the distal jaw tab 1229 being in contact with the anvil jaw 1320 when the closure tube 1220 is initially retracted. In such instances, the proximal jaw tab 1229 pulls the anvil jaw 1320 into a partially open position by itself. As the distal closure tube 1220 is retracted further, the distal jaw tab 1229 comes into contact with the anvil jaw 1320 and pulls the anvil jaw 1320 into its fully-open position during a second opening motion. In various instances, the initial jaw opening motion and the second opening motion comprise one continuous motion while, in other instances, a pause is present between the initial jaw opening motion and the second opening motion. The proximal jaw tab 1229 disengages from the anvil jaw 1320 before or when the distal jaw tab 1229 comes into contact with the anvil jaw 1320. In other embodiments, the distal jaw tab 1229 and the proximal jaw tab 1229 are simultaneously in contact with the anvil jaw 1320 for at least a portion of the second opening motion. In either event, the sequential co-operation of the two jaw opening tabs 1229 allows the anvil jaw 1320 to be opened wider than could be done with a single jaw opening tab.

Further to the above, referring again to FIG. 12, the anvil jaw 1320 is movable between open and closed positions along an axis 12-6A which represents, in FIG. 12, a line extending between the 12 o'clock and 6 o'clock positions on a clock face. As discussed above, the distal closure tube 1220 is advanced distally into contact with the anvil jaw 1320 to move the anvil jaw 1320 into a closed position. More specifically, as also discussed above, the distal closure tube 1220 is advanced distally into contact with ridges 1344 and then slides along the ridges 1344 to move and hold the anvil jaw 1320 in its fully-closed position. When patient tissue is compressed between the anvil jaw 1320 and the staple cartridge 1500, however, the anvil jaw 1320 is biased away from the staple cartridge 1500 upwardly along the 12-6A axis by the compressed patient tissue. Similarly, the staples forming against the anvil jaw 1320 during the staple firing stroke tend to push the anvil jaw 1320 away from the staple cartridge 1500. In such instances, absent more, the ridges demarcated 1344-1 (representing the 1 o'clock position of a clock face) and 1344-11 (representing the 11 o'clock position of a clock face) in FIG. 12 apply a large opening force to the distal closure tube 1210 that tends to pull the distal closure tube 1220 against the cartridge jaw 1310 and stretch, elastically and/or plastically, the distal closure tube 1220 into an ovaloid shape. More specifically, the distal closure tube 1220, which has a round shape in its unloaded state, elongates along the 12-6A axis but contracts along an orthogonal 3-9A axis. Such stretching of the distal closure tube 1220 can allow the anvil jaw 1320 to at least partially open and, in various instances, increase the force needed to advance the firing driver through the staple firing stroke.

In order to reduce, if not eliminate, the ovaloid stretching of the distal closure tube 1220, further to the above, the anvil jaw 1320 comprises ridges 1344-3 and 1344-9 which maintain, or at least substantially maintain, the round configuration of the distal closure tube 1220 seen in FIG. 12. The ridge 1344-3 represents the 3 o'clock position of a clock face in FIG. 12 and the ridge 1344-9 represents the 9 o'clock position of a clock face in FIG. 12. The ridges 1344-3 and 1344-9 comprise the outermost lateral edges of the anvil jaw 1320 and define the 3-9A axis, mentioned above, which is orthogonal to the 12-6A axis. When, or if, the distal closure tube 1220 is stretched along the 12-6A axis by the anvil jaw 1320, as discussed above, the lateral sides of the distal closure tube 1220 come into contact with and are supported by the lateral ridges 1344-3 and 1344-9 which prevent, or at least limit, the inward lateral deflection of the lateral sides of the distal closure tube 1220. As such, the circular shape of the distal closure tube 1220 seen in FIG. 12 is maintained, or at least substantially maintained, by the lateral ridges 1344-3 and 1344-9 of the anvil jaw 1320, the anvil jaw 1320 is held in its fully-closed position, and, as a result, the force needed to fire the staple cartridge 1500 is reduced.

Further to the above, there is a line-to-line fit between an outer diameter of the anvil jaw 1320 defined between the lateral ridges 1344-3 and 1344-9 and an inner aperture diameter defined by the inner surface 1227 of the distal closure tube 1220. As such, the lateral ridges 1344-3 and 1344-9 oppose the inward lateral deflection of the lateral sides of the distal closure tube 1220 through out the entire closure stroke of the distal closure tube 1220. As the reader will appreciate, however, manufacturing tolerances may, absent more, result in the outer diameter of the anvil jaw 1320 defined between the lateral ridges 1344-3 and 1344-9 being smaller than the inner aperture diameter of the distal closure tube 1220. In such instances, the lateral sides of the distal closure tube 1220 will deflect into contact with the lateral ridges 1344-3 and 1344-9 which will then oppose the continued inward deflection of the lateral sides of the distal closure tube 1220. Such instances can be acceptable; however, they can be avoided by designing the anvil jaw 1320 and the distal closure tube 1220 such that there is contact between the lateral ridges 1344-3 and 1344-9 and the distal closure tube 1220 through out the tolerance ranges of the components. Such instances can also be avoided by establishing the outer diameter defined by the lateral ridges 1344-3 and 1344-9 as a jaw reference datum used to manufacture the anvil jaw 1320 and the inner diameter of the distal closure tube 1220 as a tube reference datum used to manufacture the distal closure tube 1220. Establishing these features as reference datums during the manufacturing process can assure that the line-to-line fit therebetween is maintained.

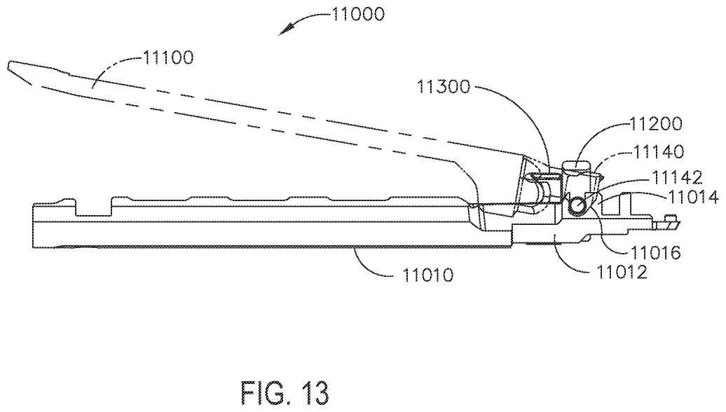

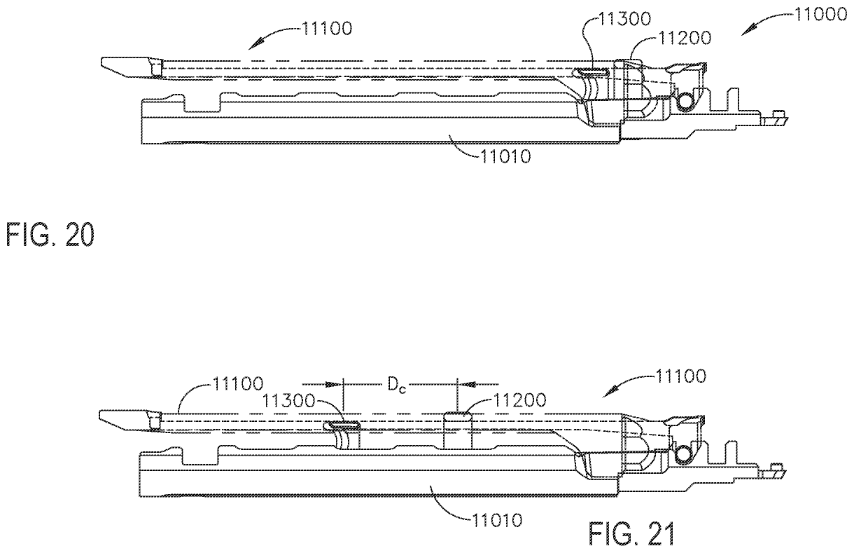

Referring to FIGS. 13-21, a surgical end effector 11000 comprises an anvil 11100 that is pivotally supported on an elongate channel 11010 that is configured to operably support a surgical staple cartridge therein. This arrangement also employs two rotary actuation shafts-one for closure, i.e., moving the anvil 11100 into a closed position, and one for firing, i.e., axially moving a firing member within the anvil 11100 and elongate channel 11010. The anvil 11100 includes an elongate anvil body 11110 and an anvil mounting portion 11140. In one arrangement, for example, the anvil 11100 is fabricated using various fabricating techniques described in U.S. patent application Ser. No. 16/105,101, entitled METHOD FOR FABRICATING SURGICAL STAPLER ANVILS. The entire disclosure of U.S. patent application Ser. No. 16/105,101, entitled METHOD FOR FABRICATING SURGICAL STAPLER ANVILS is hereby incorporated by reference herein. A pair of anvil trunnions 11142 protrude laterally from the anvil mounting portion 11140. Each anvil trunnion 11142 is pivotally supported in a corresponding trunnion cradle 11016 that is formed in a corresponding upstanding wall 11014 of a proximal end portion 11012 of the elongate channel 11010. See FIG. 13. The proximal end portion 11012 of the elongate channel 11010 is pivotally coupled to an elongate shaft assembly of a surgical instrument to facilitate the articulation of the end effector 11000. In other arrangements, the elongate channel 11010 is not capable of articulation. Each anvil trunnion 11142 is retained within its trunnion cradle 11016 by an anvil retainer (not shown).

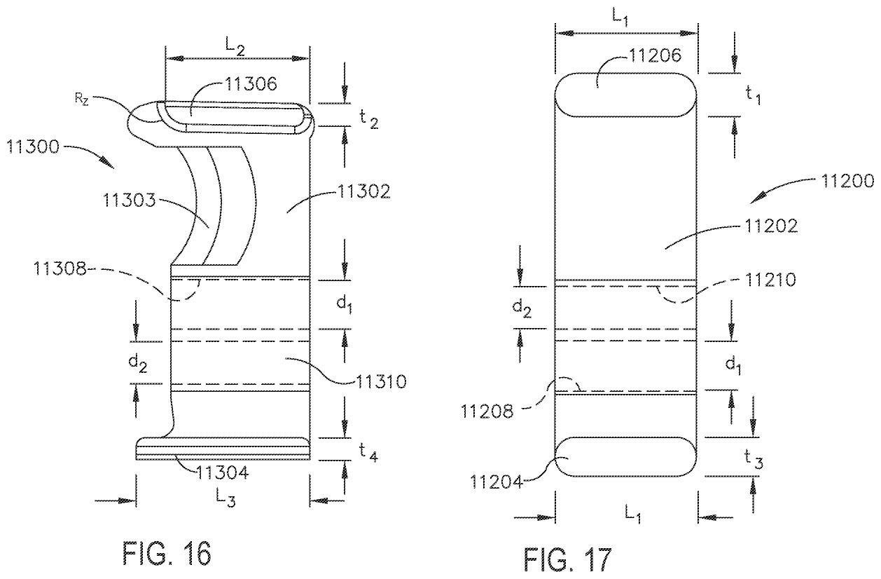

The surgical end effector 11000 includes an anvil closure member 11200 and a firing member 11300 that are each independently controlled and axially movable. FIG. 16 illustrates one form of a closure member 11200 that may be employed. As can be seen in FIG. 16, the closure member 11200 includes a vertically extending closure body 11202 that has two bottom channel tabs 11204 laterally protruding therefrom. A portion of the closure body 11202 is configured to extend through a slot in a bottom surface of the elongate channel 11010 and the channel tabs 11204 extend laterally outward to slideably engage the bottom of the channel 11010. Similarly, a pair of anvil engaging tabs 11206 protrude laterally from the top of the closure body 11202 to slideably engage the anvil 11100. The closure body 11202 includes a threaded through hole 11208 for threadably engaging a threaded portion of a rotatable closure drive shaft for axially driving the closure member 11200.

As indicated above, the surgical end effector 11000 further includes an axially movable firing member 11300. FIG. 17 illustrates one form of a firing member 11300 that may be employed. As can be seen in FIG. 17, the firing member 11300 includes a vertically extending firing member body 11302 that has a tissue cutting surface 11303 as well as two bottom channel tabs 11304 laterally protruding from the firing member body 11302. A portion of the firing member body 11302 is configured to extend through the slot 11019 in the bottom surface 11018 of the elongate channel 11010 and the channel tabs 11304 extend laterally outward to slideably engage the bottom of the channel 11010. See FIG. 17. Similarly, a pair of anvil engaging tabs 11306 protrude laterally from the top of the firing member body 11302 to slideably engage the anvil 11100.

Further to the above, the firing member body 11302 includes a threaded through hole 11308 that is threadably engaged with a threaded portion of a rotatable firing drive shaft. The firing drive shaft passes through an unthreaded clearance hole 11210 in the closure body 11202. See FIG. 17. The firing drive shaft extends axially down the elongate channel 11010 and is rotatably supported at a distal end portion thereof by a bearing (not shown) or another arrangement. Similarly, the closure drive shaft extends axially down the elongate channel 11010 and is rotatably supported at a distal end portion thereof by a bearing (not shown) or another arrangement. Thus, the firing member body 11302 similarly has an unthreaded clearance hole 11310 therethrough to accommodate the closure drive shaft. It will be appreciated that, in such arrangements, the closure drive shaft 10710 and the firing drive shaft 10610 may be supported in a vertical stacked arrangement so that they may be independently rotatable.

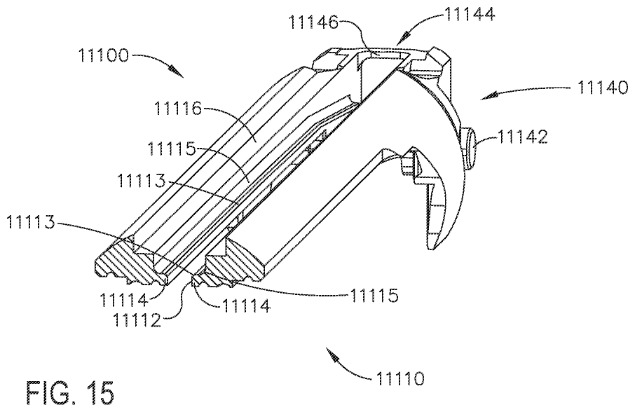

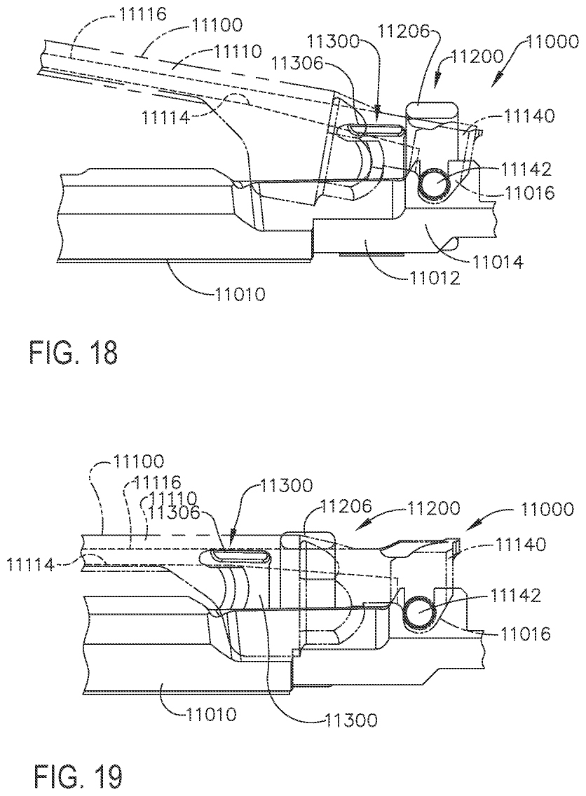

Referring to FIG. 18, the anvil mounting portion 11140 of the anvil 11100 includes a central cross brace 11144 that serves to define an opening 11146 for accommodating the closure member 11200 therein when the closure member 11200 is in its proximal-most position which corresponds to an open position of the anvil 11100. As can be seen in FIG. 18, the anvil body 11110 defines an elongate slot 11112 for accommodating the firing member body 11302 and closure body 11202 therethrough. The firing member 11300 is located distal to the closure member 11200. The anvil engagement tabs 11306 on the firing member 11302 are configured to slideably engage corresponding first or lower ledges 11114 that are formed on each side of the slot 11112. As can be seen in FIGS. 18 and 19, the lower ledges 11114 taper slightly downward at their proximal ends to accommodate the anvil engagement tabs 11306 on the firing member 11300 when the anvil is pivoted to its open position. The anvil engagement tabs 11206 on the closure member 11200 are configured to slideably engage corresponding second or higher ledges 11116 that are formed on each side of the slot 11112. See FIG. 15. To open the anvil 11100, the closure drive shaft is rotated to threadably drive the closure member 11200 proximally into its proximal-most position (FIG. 18). When the closure member 11200 is in its proximal-most position, the anvil engagement tabs 11206 thereon apply a pivotal opening motion to the anvil 11100 to pivot the anvil open. The firing member 11300 is in its starting position so that the anvil engagement tabs 11306 of the firing member 11300 do not apply a closure motion to the anvil 11100.

To close the anvil 11100, the closure drive shaft is rotated in an opposite direction to distally advance the closure member 11200 into a closed position. The firing drive shaft may also be simultaneously rotated to distally advance the firing member 11300 into a starting position. When the closure member 11200 and the firing member 11300 are in those positions, the anvil 11100 is closed and the firing member 11300 is ready to be fired. Thus, assuming that an unspent surgical staple cartridge has been first operably supported in the elongate channel 11010 and the end effector 11000 was manipulated to capture the target tissue between the staple cartridge and the anvil, the user may close the anvil 11100 onto the tissue in the above described manner to ready the end effector to be fired. During this closing process, the firing drive shaft is rotated to drive the firing member 11300 distally into the clamped tissue to cut the tissue and cause the staples stored in the staple cartridge to be formed into the cut tissue on both sides of the cut. During this process, the closure member 11200 may also be driven distally to apply additional closure motions to the anvil 11100 and elongate channel 11010. Depending upon the amount of resistance experienced by the firing member 11300, for example, the closure member 11200 can be advanced with the firing member 11300, stop and then go again. The closure member 11200 may be advanced distally at a different rate from the firing member's rate of distal advancement. The distance Dc between the closure member 11200 and the firing member 11300 may be controlled to balance the loads experienced during the firing process. See FIG. 21. For example, if the user wanted to decrease an amount of vertical load being experienced by the firing member 11300, the closure member 11200 could be moved closer to the firing member 11300 during advancement. The vertical loads experienced by the firing member 11300 may be increased by increasing the distance between the firing member 11300 and the closure member 11200.

Returning to FIGS. 16 and 17, the thickness t₁ of the anvil engagement tabs 11206 on the closure member 11200 is greater than the thickness t₂ of the anvil engagement tabs 11306 on the firing member 11300. In one arrangement, the length L₁ of the anvil engagement tabs 11206 on the closure member 11200 is slightly less than the length L₂ of the anvil engagement tabs 11306 on the firing member 11300. Likewise, the thickness t₃ of the channel tabs 11204 on the closure member 11200 is greater than the thickness t₄ of the channel tabs 11304 on the firing member 11300. In one arrangement, the length L₁ of the channel tabs 11204 on the closure member 11200 are shorter than the length L₃ of the channel tabs 11304 on the firing member 11300. In both cases, the diameters d₁ of the threaded holes 11208, 11308 may be greater than the diameters d₂ of the unthreaded through holes 11210, 11310. In addition, the relative attack angles between the anvil engagement tabs 11206, 11306 and their corresponding anvil ledges and the channel tabs 11204, 11304 and their corresponding channel ledges may be varied, the same or different. In one arrangement, the anvil engagement tabs 11306 on the firing member 11300 are arranged at a slightly higher attack angle relative to their corresponding anvil ledges than the attack angle of the anvil engagement tabs 11206 on the closure member 11200. In one arrangement, the channel tabs 11204 and 11304 ride on the same ledges that are formed in the bottom of the elongate channel 11010. See FIG. 13. The closure member 11200 and the firing member 11300 have separate acting paths which can permit the closure member to be designed to accommodate larger moment arms from the anvil pivot for better firing efficiency.

One advantage that may be experienced when using the foregoing configuration is that the closure member 11200 can be moved away from the firing member 11300 to gain a significant amount of mechanical advantage during closure. The closure member 11200 does not need to travel the complete length of the firing stroke. For example, if the closure member 11200 were to be advanced about half way down the end effector, the relative stiffness of the anvil 11100 would reduce the amount of load being encountered by the firing member 11300. A control system employing sensors (e.g., strain gauges, etc.) for detecting amounts of loads being experienced by the firing system components and closure system components, as well as algorithms, can be used to balance the loads being encountered by both systems. For example, a maximum threshold of vertical load experienced by the firing member 11300 can be set in the controller based on the geometry and composition of that firing member component. When the load approaches that threshold, the algorithm can automatically advance the closure member 11200 so that it absorbs more of the load and reduces the amount of load being experienced by the firing member 11300. In various aspects, as the firing member 11300 is distally driven through the surgical staple cartridge, the firing member 11300, through the engagement of the anvil engagement tabs 11306 with the anvil 11100 and the engagement of the channel engagement tabs 11304 with the channel 11010, may serve to maintain a desired amount of tissue gap between a deck surface on the staple cartridge and a staple forming undersurface on the anvil 11100. Other closure control methods may also be employed in connection with opening and closing the end effector such as those disclosed in U.S. patent application Ser. No. 16/105,081, entitled METHOD FOR OPERATING A POWERED ARTICULATABLE SURGICAL INSTRUMENT. The entire disclosure of U.S. patent application Ser. No. 16/105,081, entitled METHOD FOR OPERATING A POWERED ARTICULATABLE SURGICAL INSTRUMENT is hereby incorporated by reference herein. The entire disclosure of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, is incorporated by reference herein. The entire disclosure of U.S. Pat. No. 6,978,921, entitled SURGICAL STAPLING INSTRUMENT INCORPORATING AN E-BEAM FIRING MECHANISM, which issued on Dec. 27, 2005, is incorporated by reference herein.

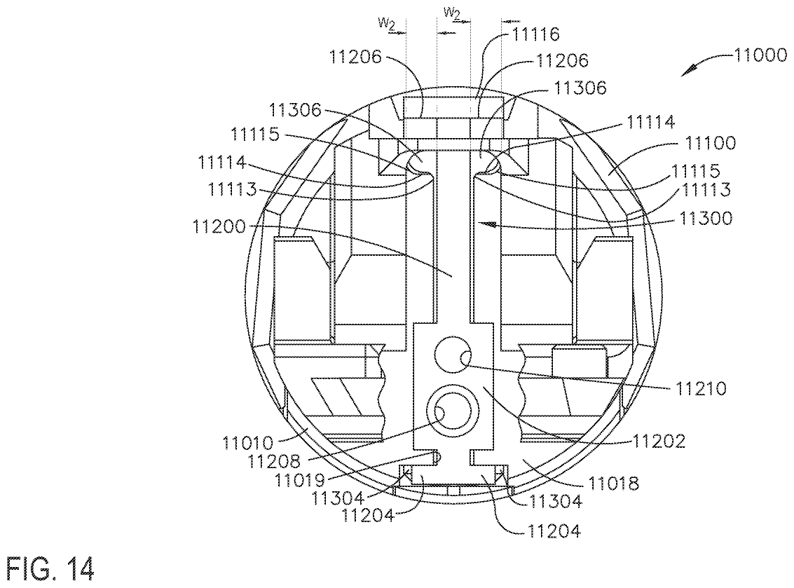

As discussed above, referring again to FIGS. 14, 15 and 17, the firing member 11300 engages the lower ledges 11114 of the anvil 11100 during a staple firing stroke to hold the anvil 11100 in position relative to the cartridge jaw 11010. As also discussed above, the firing member 11300 has two laterally-extending engagement tabs 11306—one that extends in a first lateral direction to engage a first lower ledge 11114 and another that extends in a second, or opposite, lateral direction to engage a second lower ledge 11114. Each engagement tab 11306 extends from the firing member body 11302 a width w₂. Notably, the width w₂ of each engagement tab 11306 is shorter than the length L₁. In various embodiments, the width w₂ of each engagement tab 11306 is between 15% and 25% of length L₁, between 20% and 30% of the length L₁, or between 25% and 35% of the length L₁, for example. As a result, each engagement tab 11306 has the configuration of a stubby flange that resists being deflected during the staple firing stroke. More specifically, the staples, when fired, are driven against the anvil 11100 during the staple firing stroke which tends to push the anvil 11100 away from the cartridge jaw 11010 and deflect the engagement tabs 11306 upwardly, i.e., away from the cartridge jaw 11010. Given the stubby configuration of the engagement tabs 11306, however, the engagement tabs 11306 resist the upward deflection thereof and, as a result, resist the upward movement of the anvil 11100 away from its fully-closed position during the staple firing stroke. As a result, the anvil 11100 can remain in its fully-closed position during the staple firing stroke and the staple firing drive does not need to transmit an excessive firing force into the firing member 11300 to push the anvil 11100 back into its fully-closed position as the firing member 11300 is moved distally.

As discussed above, referring again to FIGS. 14, 15 and 17, each engagement tab 11306 extends laterally from the firing member body 11302. A fillet radius R₂ is present at the interconnection between each engagement tab 11306 and the firing member body 11302 which resists the upward deflection of the engagement tab 11306 during the staple firing stroke. In various embodiments, the fillet radius R₂ is between ¼ and ⅓ of the engagement tab thickness t₂, between ⅕ and ⅓ of the engagement tab thickness t₂, or between ⅕ and ⅓ of the engagement tab thickness t₂, for example. The fillet radius R₂ is present at a location with a high amount of strain and stress when the engagement tab 11306 is being bent upwardly. The fillet radius R₂ relieves part of that strain. Moreover, the anvil 11100 further comprises a radiused edge 11113 extending along each lower ledge 11114 that matches, or at least substantially matches, the fillet radius R₂ of the firing member 11300. In various embodiments, the matched radiused edge 11113 is defined by the fillet radius R₂. In other embodiments, matched radiused edge 11113 is defined by a radius that is within 10% larger or within 10% smaller than the radius R₂, for example. In any such embodiment, the fillet radius R₂ of each engagement tab 11306 is supported by its respective radiused edge 11113. In at least one embodiment, the fillet radius R₂ of each engagement tab 11306 is fully supported by its respective radiused edge 11113. Such support limits the upward deflection of the engagement tabs 11306 and, as a result, resists the upward movement of the anvil 11100 from its fully-closed position during the staple firing stroke and, thus, reduces the firing force needed to perform the staple firing stroke.

Further to the above, referring again to FIGS. 14, 15 and 17, each engagement tab 11306 comprises a lateral end unconnected to the firing member body 11302. Each lateral end is defined by a curved surface. In various embodiments, each lateral end is defined by a single radius of curvature. In other embodiments, each lateral end is defined by more than one radius of curvature. In at least one embodiment, each lateral end comprises a spherical shape, for example. Moreover, the anvil 11100 further comprises a radiused corner 11115 extending along each lower ledge 11114 that matches, or at least partially matches, the profile of the lateral end of an engagement tab 11306. In various embodiments, each matched radiused corner 11115 is defined by a radius that defines a lateral end of an engagement tab 11306. In other embodiments, the matched radiused corner 11115 is defined by a radius that is between 10% larger and 10% smaller than a radius defining a lateral end of an engagement tab 11306, for example. In any such embodiment, the lateral end of each engagement tab 11306 is supported by its respective radiused corner 11115. Such support limits the upward deflection of the engagement tabs 11306 and, as a result, resists the upward movement of the anvil 11100 from its fully-closed position during the staple firing stroke and, thus, reduces the firing force needed to perform the staple firing stroke.

In at least one embodiment, a surgical stapling instrument includes the anvil closure system of the surgical stapling instrument 1000 and the staple firing system of the surgical stapling instrument 11000. Such a surgical stapling instrument includes the closure tube 1210/1220 that is advanced distally to engage contact ridges 1344 defined on the outside of the anvil and, also, the firing member 11300 that engages the lower ridges 11114 and is supported by the radiused edges 11113 and the radiused corners 11115. Such an arrangement advantageously and co-operatively limits the upward movement of the anvil before and during the staple firing stroke.

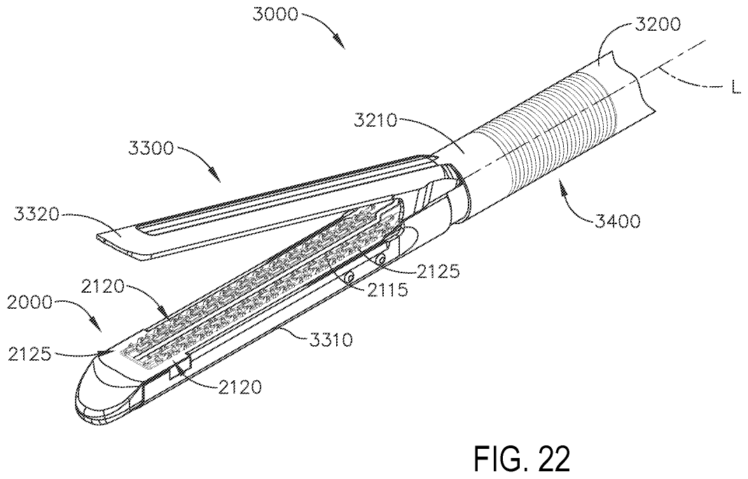

A surgical stapling instrument 3000 is illustrated in FIG. 22. The stapling instrument 3000 comprises a handle, an elongate shaft 3200 extending from the handle, and an end effector 3300 including a cartridge jaw 3310 and an anvil jaw 3320. The handle comprises triggers that are actuatable by a clinician to operate the stapling instrument 3000 as described further below. In various other embodiments, the stapling instrument 3000 comprises a housing assembly configured to be attached to a robotic surgical instrument system instead of the handle. In at least one such embodiment, the housing assembly comprises rotatable inputs that are operably coupled with motor-driven outputs of the robotic surgical instrument system when the housing assembly is attached to the robotic surgical instrument system. The robotic surgical instrument system comprises a control station including triggers that are actuatable by a clinician to operate the surgical instrument 3000. The entire disclosure of U.S. Pat. No. 9,072,535, entitled SURGICAL STAPLING INSTRUMENT WITH ROTATABLE DEPLOYABLE ARRANGEMENTS, which issued on Jul. 7, 2015, is incorporated by reference herein.

The elongate shaft 3200 is rotatable relative to handle about a longitudinal axis L. When the elongate shaft 3200 is rotated relative to the handle, the end effector 3300 rotates with the elongate shaft 3200. The handle comprises a rotation actuator mounted to the elongate shaft 3200 that is rotatable relative to the handle by the clinician to rotate the shaft 3200 about the longitudinal axis L. In various embodiments, the stapling instrument 3000 comprises a motor-driven system that is operable to rotate the elongate shaft 3200. In at least one such embodiment, the motor-driven system comprises an electric motor mounted in the handle that includes an output gear meshingly engaged with a ring of gear teeth defined on the elongate shaft 3200. The motor-driven system further comprises a trigger, such as a switch, for example, accessible by the clinician operating the stapling instrument 3000.

The end effector 3300 is rotatable relative to the shaft 3200 about an articulation joint 3400. The articulation joint 3400 defines an articulation axis about which the end effector 3300 is articulated relative to the shaft 3200. The articulation joint 3400 also defines a plane within which the end effector 3300 is articulated relative to the shaft 3200. The stapling instrument 3000 further comprises an articulation drive system including an articulation driver engaged with the end effector 3300, an electric motor operable to drive the articulation driver longitudinally, and a motor control circuit including a trigger, such as a switch, for example, accessible by the clinician operating the stapling instrument 3000. When the articulation driver is translated distally by the articulation drive system, the end effector 3300 rotates in a first direction and, when the articulation driver is translated proximally by the articulation drive system, the end effector 3300 rotates in a second, or opposite, direction. In various other embodiments, the articulation joint 3400 defines more than one articulation axis, such as two articulation axes, for example, about which the end effector 3300 can be rotated relative to the shaft 3200. In at least one embodiment, the articulation joint 3400 comprises a flexible articulation region. The entire disclosure of U.S. Pat. No. 9,629,629, entitled CONTROL SYSTEMS FOR SURGICAL INSTRUMENTS, which issued on Apr. 25, 2017, is incorporated by reference herein.

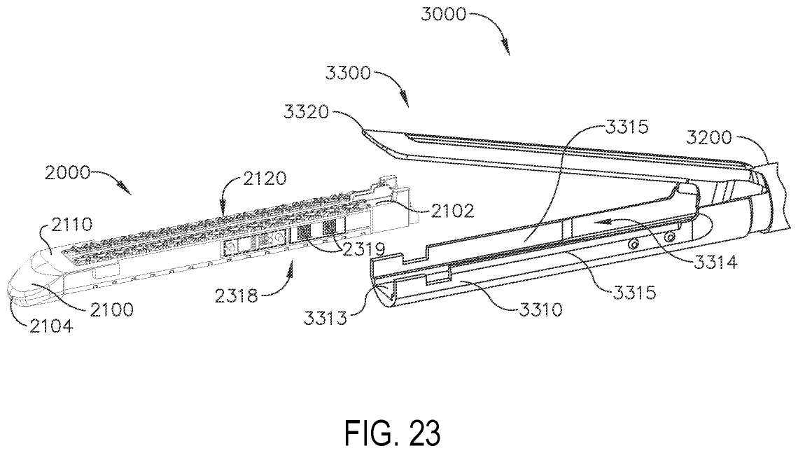

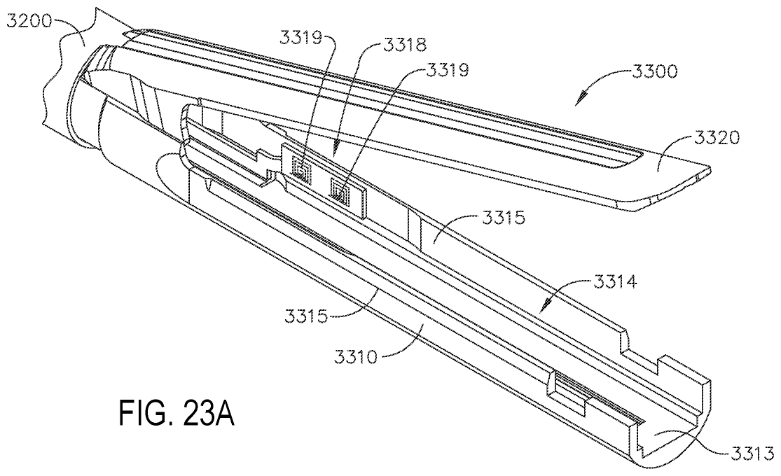

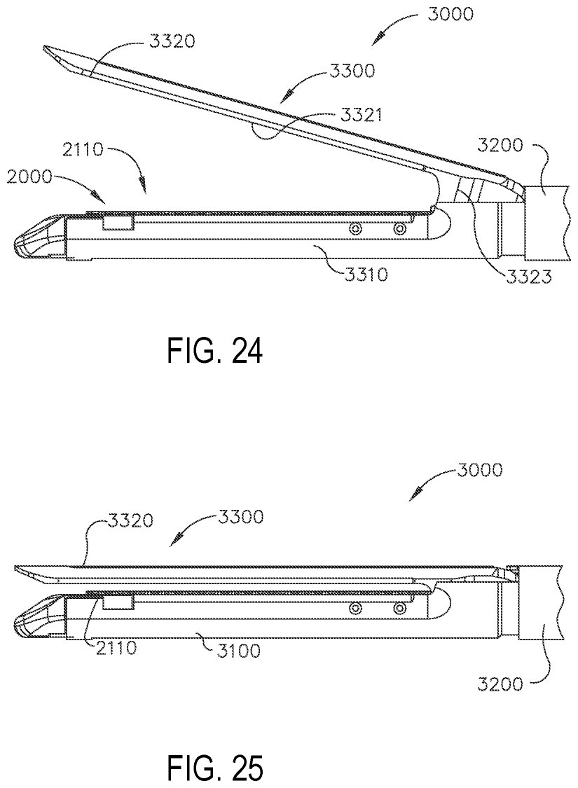

Referring to FIG. 23, the cartridge jaw 3310 of the end effector 3300 comprises a channel 3314 configured to receive a replaceable staple cartridge 2000, for example. The channel 3314 comprises a bottom 3313 and lateral sidewalls 3315 extending upwardly from the bottom 3313. The staple cartridge 2000 comprises a cartridge body 2100 including a proximal end 2102, a distal end 2104, a deck 2110 configured to support patient tissue thereon, and longitudinal rows of staple cavities 2120 defined in the deck 2110. Referring to FIGS. 24 and 25, the anvil jaw 3320 of the end effector 3300 is rotatably connected to the cartridge jaw 3310 such that the anvil jaw 3320 is rotatable about a pivot axis. The anvil jaw 3320 comprises a tissue compression surface 3321 that is brought into opposition with the deck 2110 of the staple cartridge 2000 when the anvil jaw 3320 is rotated from a fully-open position (FIG. 24) into a fully-closed position (FIG. 25). The above being said, alternative embodiments are envisioned in which the cartridge jaw 3310 is movable relative to the anvil jaw 3320.

Further to the above, the anvil jaw 3320 is movable from a fully-open position (FIG. 24) to a fully-closed position (FIG. 25) to clamp tissue between the anvil jaw 3320 and the staple cartridge 2000. The stapling instrument 3000 further comprises a jaw closure system configured to move the anvil jaw 3320 into its fully-closed position. The jaw closure system comprises a trigger rotatably coupled to the handle, a closure carriage positioned in the handle translatable distally by the trigger, and a closure tube 3210 supported by the closure carriage such that the closure tube 3210 translates with the closure carriage. The trigger comprises a rotatable lever movable from an unactuated position to an actuated position to transmit a force applied to the trigger by a clinician to the closure tube 3210. When the trigger is pulled toward a grip of the handle by the clinician, the trigger pushes the closure carriage and the closure tube 3210 distally.

In various alternative embodiments, the jaw closure system comprises an electric motor configured to drive the closure tube 3210 distally through the closure stroke when the electric motor is operated in a first direction and retract the closure tube 3210 proximally when the electric motor is operated in a second, or opposite, direction. In at least one such embodiment, the jaw closure system comprises a motor control circuit configured to control the operation of the electric motor. The motor control circuit comprises a processor and a trigger that is actuatable by the clinician to operate the electric motor. That said, any suitable motor actuation system can be utilized.

The staple cartridge 2000 and/or the cartridge jaw 3310 comprise features which releasably lock the staple cartridge 2000 in the cartridge jaw 3310 such that the staple cartridge 2000 is secured, or seated, in position in the cartridge jaw 3310 but can be replaced during a surgical procedure. In various other embodiments, the staple cartridge 2000 is secured in the cartridge jaw 3310 in a manner that does not allow the staple cartridge 2000 to be released from the cartridge jaw 3310 and replaced during a surgical procedure. The entire disclosure of U.S. Pat. No. 11,045,191, entitled METHOD FOR OPERATING A SURGICAL STAPLING SYSTEM, which issued on Jun. 29, 2021 is incorporated by reference herein.

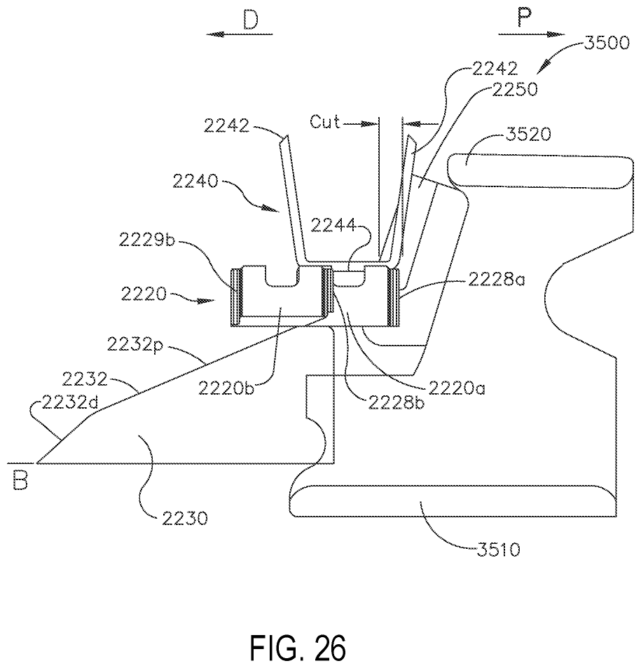

Referring primarily to FIG. 26, the staple cartridge 2000 further comprises staples 2240 stored in the staple cavities 2120 and staple drivers 2220 movable within the staple cavities 2120 to eject the staples 2240 from the staple cavities 2120 during a staple firing stroke. As discussed further below, each staple driver 2220 is configured to support and drive, i.e., fire, three staples 2240; however, other embodiments are envisioned in which one or more staple drivers of the staple cartridge 2000 are configured to fire more than three staples or less than three staples. Referring to FIG. 26, the staple cartridge 2000 further comprises a sled 2230 movable from a proximal position to a distal position by a firing driver 3500 of the stapling instrument 3000 during the staple firing stoke to lift the staple drivers 2220 within the staple cavities 2120 and eject the staples 2240 from the staple cartridge 2000.

Further to the above, referring again to FIG. 26, the sled 2230 comprises at least one ramp 2232 configured to engage and slide under the staple drivers 2220 as the sled 2230 is moved distally during the staple firing stroke. As the ramp 2232 passes under a staple driver 2220, the ramp 2232 moves the staple driver 2220 upwardly within three staple cavities 2120 so that the staples 2240 positioned within the three staple cavities 2120 are simultaneously driven upwardly toward the anvil jaw 3320 positioned opposite the staple cartridge 2000. In such instances, the staple driver 2220 moves from an unfired position at the bottom of the staple cavities 2120 to a fired position in which the staple driver 2220 is supported by the top, or apex, of the ramp 2232 as illustrated in FIG. 26. Once the staple driver 2220 has reached the fully-fired position illustrated in FIG. 26, the three staples driven by the staple driver 2220 have been deformed into their fully-fired or fully-deformed configuration. In various instances, such a fully-fired configuration of the staples 2240 comprises a B-shaped configuration, for example, in which the legs 2242 of the staples 2240 have been bent inwardly and downwardly toward their bases 2244. Notably, the staple 2240 in FIG. 26 is depicted in its unfired configuration—and not its fired configuration. In this fully-fired position of the staple driver 2220, however, the reader should appreciate that the staple 2240 will have already been deformed into its fully-fired or deformed configuration.

Referring again to FIG. 26, the sled 2230 further comprises a tissue cutting knife 2250 incise patient tissue during the staple firing stroke. The tissue cutting knife 2250 comprises a distally-presented knife edge that moves within a longitudinal slot 2115 defined in the cartridge body 2100. Prior to the staple firing stroke, the sled 2230 is positioned in an unfired position at the proximal end 2102 of the staple cartridge 2000. In this position, the tissue cutting knife 2250 extends above the deck 2110 of the cartridge body 2100 between two knife guards extending upwardly from the deck. As the sled 2230 is advanced distally toward the distal end 2104 of the staple cartridge 2000 during the staple firing stroke, the tissue cutting knife 2250 is exposed and can cut the patient tissue captured between the staple cartridge 2000 and the anvil jaw 3320. In various other embodiments, the sled of the staple cartridge 2000 does not include a tissue cutting knife. In at least one such embodiment, the firing driver 3500, for example, comprises a tissue cutting knife.

The anvil jaw 3320 further comprises staple forming pockets arranged in longitudinal rows that are registered with the staple cavities 2120 defined in the staple cartridge 2000 when the anvil jaw 3320 is in its fully-closed position. The anvil jaw 3320 further comprises tissue stops 3323 that extend downwardly toward the cartridge jaw 3310 and are positioned inwardly with respect to the lateral sides of the cartridge jaw 3310. The tissue stops 3323 inhibit the migration of tissue proximally into the end effector 3300 past the tissue stops 3323 which can reduce the possibility of the tissue coming into contact with a tissue cutting edge of the tissue cutting knife 3250 parked in its proximal unfired position.

Referring again to FIG. 26, the firing driver 3500 comprises a first cam 3510 configured to engage the cartridge jaw 3310 during the staple firing stroke. Similarly, the firing driver 3500 comprises a second cam 3520 configured to engage the anvil jaw 3320 during the staple firing stroke. During the staple firing stroke, the first cam 3510 and the second cam 3520 position the anvil jaw 3320 relative to the staple cartridge 2000 in the cartridge jaw 3310 such that the staples 2240 ejected from the staple cartridge 2000 contact the forming pockets defined in the anvil jaw 3320 and are deformed to a suitable deformed height, or a deformed height within a suitable height range. Notably, the second cam 3520 is positioned vertically above the first cam 3510 and at least a portion of the tissue cutting knife 2250 is positioned vertically intermediate the first cam 3510 and the second cam 3520. Moreover, notably, at least a portion of the staple 2240 depicted in FIG. 26 is positioned distally with respect to the tissue cutting knife 2250. Although not depicted in FIG. 26, the staple driver 2220 is configured to support two additional staples 2240—one staple 2240 that is laterally aligned with and parallel to the depicted staple 2240 and another staple 2240 that is positioned distally with respect to the two laterally aligned staples 2240. As a result of the staples 2240 being positioned distally, or at least partially distally, with respect to the tissue cutting knife 2250, the patient tissue is stapled before it is transected by the tissue cutting knife 2250 during the staple firing stroke.

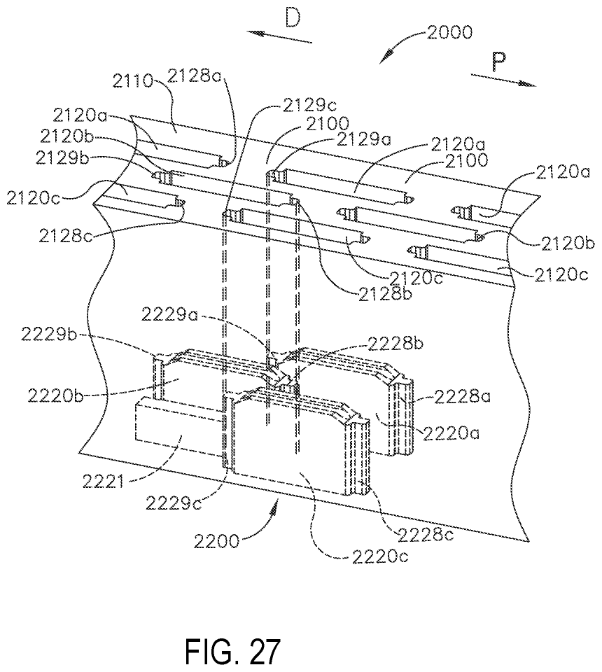

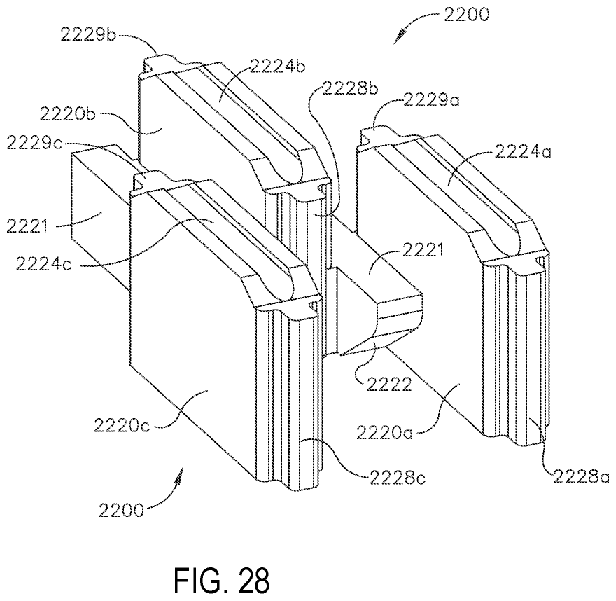

Referring again to FIG. 22, the cartridge body 2100 of the staple cartridge 2000 comprises six longitudinal rows of staple cavities 2120—three longitudinal rows on a first side of the longitudinal slot 2115 and three longitudinal rows on a second, or opposite, side of the longitudinal slot 2115. The staple drivers 2220 are arranged in two longitudinal rows—a first row on the first side of the longitudinal slot 2115 and a second row on the second side of the longitudinal slot 2115. Referring to FIGS. 27 and 28, each staple driver 2220 comprises three drive pillars—a first drive pillar 2220a, a second drive pillar 2220b, and a third drive pillar 2220c—arranged in an arrow-like, or echelon-like, arrangement. The first drive pillar 2220a is positioned within a first staple cavity 2120a in a first longitudinal row, the second drive pillar 2220b is positioned within a second staple cavity 2120b in a second longitudinal row, and the third drive pillar 2220c is positioned within a third staple cavity 2120c in a third longitudinal row. The first pillar 2220a and the second pillar 2220b are connected by a connector 2221 and, similarly, the second pillar 2220b and the third pillar 2220c are connected by another connector 2221. The second pillar 2220b is positioned distally, at least partially, with respect to the first pillar 2220a and the third pillar 2220c. The first pillar 2220a is positioned laterally with respect to and aligned with the third pillar 2220c. Each drive pillar comprises a seat, or cradle, that is configured to receive a base 2244 of a staple 2240 and drive the staple 2240 upwardly as the staple driver 2220 is lifted upwardly by the sled 2230. More specifically, the first drive pillar 2220a comprises a first cradle 2224a configured to receive and drive the base 2244 of a first staple 2240, the second drive pillar 2220b comprises a second cradle 2224b configured to receive and drive the base 2244 of a second staple 2240, and the third drive pillar 2220c comprises a third cradle 2224c configured to receive and drive the base of a third staple 2240.

In each longitudinal row of staple drivers 2220, the staple drivers 2220 are arranged in a nested manner wherein the first drive pillars 2220a, and the first cradles 2224a defined thereon, are longitudinally aligned with one another such that the staples supported by and driven by the first drive pillars 2220a during the staple firing stroke form a first longitudinal line of staples in the patient tissue. Similarly, the second drive pillars 2220b, and the second cradles 2224b defined thereon, are longitudinally aligned with one another such that the staples supported by and driven by the second drive pillars 2220b during the staple firing stroke form a second line of staples in the patient tissue. Also, similarly, the third drive pillars 2220c, and the third cradles 2224c defined thereon, are longitudinally aligned with one another such that the staples supported by and driven by the third drive pillars 2220c during the staple firing stroke form a third line of staples in the patient tissue.

As described above, the sled 2230 is moved distally during the staple firing stroke to engage the staple drivers 2220 and drive the staple drivers 2220 toward the anvil jaw 3120 positioned opposite the staple cartridge 2000. At the outset of the staple firing stroke, the sled 2230 comes into contact with the proximal-most staple driver 2220 in the first longitudinal row of staple drivers 2220 and the proximal-most staple driver 2220 in the second longitudinal row of staple drivers 2220 and lifts these two proximal-most drivers 2220 toward the anvil jaw 3120. As the sled 2230 is moved further distally by the firing driver 3500 during the staple firing stroke, the sled 2230 sequentially engages the remainder of the staple drivers 2220 in the longitudinal rows of staple drivers 2220 until all of the staple drivers 2220 have been lifted to their fully-fired positions and/or the staple firing stroke is stopped early and the firing driver 3500 is retracted. As a result of the above, the staple drivers 2220 within a longitudinal row of staple drivers 2220 move relative to one another during the staple firing stroke. When the staple drivers 2220 are in their unfired positions, the second pillars 2220b of the staple drivers 2220 are positioned intermediate, or at least partially positioned intermediate, the first and third pillars 2220a, 2220c of the staple driver 2220 positioned distally in front of it owing to the nested arrangement of the staple drivers 2220 within a longitudinal row. As a staple driver 2220 is lifted relative to the staple driver 2220 positioned distally in front of it, the staple drivers 2220 un-nest and then re-nest when the staple drivers 2220 reach their fired positions. Each staple driver 2220 comprises two ramp, or inclined, surfaces 2222—one on each connector 2221—that are engaged by the sled 2230 during the staple firing stroke to lift the staple driver 2220 as described above.

Referring again to FIGS. 27 and 28, the first pillar 2220a of each staple driver 2220 comprises a proximal guide rail 2228a extending proximally from the proximal end of the cradle 2224a and a distal guide rail 2229a extending distally from the distal end of the cradle 2224a. Each proximal guide rail 2228a is slideably received in a proximal rail slot 2128a of a first staple cavity 2120a and, likewise, each distal guide rail 2229a is slideably received in a distal rail slot 2129a. Similarly, the second pillar 2220b of each staple driver 2220 comprises a proximal guide rail 2228b extending proximally from the proximal end of the cradle 2224b and a distal guide rail 2229b extending distally from the distal end of the cradle 2224b. Each proximal guide rail 2228b is slideably received in a proximal rail slot 2128b of a first staple cavity 2120b and, likewise, each distal guide rail 2229b is slideably received in a distal rail slot 2129b. Also, similarly, the third pillar 2220c of each staple driver 2220 comprises a proximal guide rail 2228c extending proximally from the proximal end of the cradle 2224c and a distal guide rail 2229c extending distally from the distal end of the cradle 2224c. Each proximal guide rail 2228c is slideably received in a proximal rail slot 2128c of a third staple cavity 2120c and, likewise, each distal guide rail 2229c is slideably received in a distal rail slot 2129c.

Further to the above, referring again to FIGS. 26 and 28, the ramps 2232 of the sled 2230 are configured to engage the ramp surfaces 2222 of the staple drivers 2220 and push the staple drivers 2220 upwardly within the staple cavities 2120. The sled 2230 comprises four ramps 2232—two ramps 2232 on a first side of the sled 2230 that engage the staple drivers 2220 in the first longitudinal row of staple drivers 2220 and two ramps 2232 on a second, or opposite, side of the longitudinal slot 2115 that engage the staple drivers 2220 in the second longitudinal row of staple drivers 2220. The interface between the ramps 2232 of the sled 2230 and the ramp surfaces 2222 of a staple driver 2220 creates a reaction force that pushes the staple driver 2220 upwardly within its staple cavity 2210 but also pushes the staple driver 2220 distally, or longitudinally, into contact with the distal sidewalls of the staple cavity 2210. As a result of the longitudinal component of this reaction force, absent other considerations, referring again to FIGS. 27 and 28, the distal guide rail 2229a of the staple driver 2220 may engage the distal end wall of the distal rail slot 2129a of the cartridge body 2100, the distal guide rail 2229b of the staple driver 2220 may engage the distal end wall of the distal rail slot 2129b of the cartridge body 2100, and/or the distal guide rail 2229c of the staple driver 2220 may engage the distal end wall of the distal rail slot 2129c of the cartridge body 2100. Such contact between the distal guide rails 2229a, 2229b, 2229c and the distal end walls of the distal rail slots 2129a, 2129b, 2129c controls the motion of the staple driver 2220 and could, absent more, create large friction forces, and possibly binding, between the staple driver 2220 and the cartridge body 2100. In such instances, the staple driver 2220 can still be lifted to fire the staples supported thereon during the staple firing stroke, but the force needed to push the sled 2230 distally to lift the staple driver, or drivers, 2220 experiencing such friction and/or binding could be high.

In various embodiments, further to the above, the staple drivers 2220 in a longitudinal row of staple drivers 2220 support each other longitudinally. In at least one such embodiment, for instance, a staple driver 2220 being lifted by the sled 2230 is shifted into contact with the staple driver 2220 positioned distally in front of it. In at least one alternative embodiment, the staple drivers 2220 are in contact with one another without having to be shifted distally by the sled 2230. In such embodiments, longitudinally-adjacent staple drivers 2220 are in contact with one another in their unlifted positions. In either event, in one embodiment, the distal rails 2229a of the staple drivers 2220 are in contact with the distal ends of the distal rail slots 2129a and the distal rails 2229c of the staple drivers 2220 are in contact with the distal ends of the distal rail slots 2129c as the staple drivers 2220 are lifted from their unfired position to their fired position. In this embodiment, the distal rails 2229b of a staple driver 2220 being lifted by the sled 2230 is in contact with the staple driver 2220 positioned distally in front of it. More specifically, the distal rail 2229b of the staple driver 2220 being lifted by the sled 2230 is in sliding contact with the proximal rail 2228b of the staple driver 2220 positioned distally in front of it. In such instances, the rails 2228b and 2229b of the drivers 2220 comprise control surfaces. In at least one alternative embodiment, the distal rail 2229a of each staple driver 2220 slidingly engages the proximal rail 2228a of the staple driver 2220 positioned distally in front of it and, similarly, the distal rail 2229c of each staple driver 2220 slidingly engages the proximal rail 2228c of the staple driver 2220 positioned in front of it. In at least one such embodiment, the distal rails 2229b of the staple drivers 2220 are slidingly engaged with the cartridge body 2100. In such instances, the rails 2228a, 2229a, 2228c, and 2229c of the drivers 2220 comprises control surfaces.

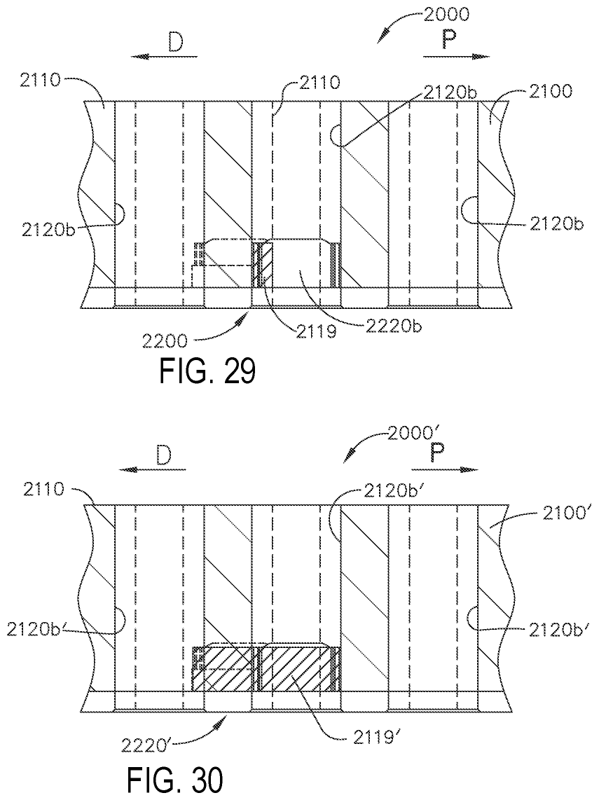

In various embodiments, further to the above, referring to FIG. 27, the interaction surfaces between the staple drivers 2220 and the sidewalls of the staple cavities 2120 are controlled, or selected, so as to lower the force needed to lift the staple drivers 2220 within the staple cavities 2120. More specifically, in at least one embodiment, the staple drivers 2220 and the staple cavities 2120 are configured and arranged such that the distal guide rails 2229a of the staple drivers 2220 are in contact with the distal ends of the distal rail slots 2129a of the staple cavities 2120 as the staple drivers 2220 are moved from their unfired positions to their fired positions during the staple firing stroke. Similarly, the distal guide rails 2229c of the staple drivers 2220 are in contact with the distal ends of the distal rail slots 2129c of the staple cavities 2120 as the staple drivers 2220 are moved from their unfired positions to their fired positions during the staple firing stroke. That said, the staple drivers 2220 and the staple cavities 2120 are configured and arranged such that the proximal guide rails 2228b of the staple drivers 2220 are in contact with the proximal ends of the proximal rail slots 2128b of the staple cavities 2120 as the staple drivers 2220 are moved from their unfired positions to their fired positions during the staple firing stroke. As such, the guide rails 2229a, 2228b, and 2229c of the staple drivers 2220 and the guide rail slots 2129a, 2128b, and 2129c of the staple cavities 2120 comprise control surfaces which constrain the longitudinal movement of the staple drivers 2220 as the staple drivers 2220 are engaged by the sled 2230. In various instances, at least one portion of the staple drivers 2220 is flexible so as to permit the guide rails 2229a, 2228b, and 2229c to be in simultaneous contact with the ends of the guide rail slots 2129a, 2128b, and 2129c, respectively. In at least one embodiment, referring to FIG. 29, the connectors 2221 of each staple driver 2220 are sufficiently flexible to permit the pillars 2220a, 2220b, and 2220c of the staple driver 2220 to engage the cartridge body 2100 in a bearing zone 2119 (represented by a cross-hatched area). In at least one such embodiment, each staple driver 2220 is comprised of a unitary piece of material, such as plastic, for example, and the connectors 2221 comprise cutouts defined therein which permit the connects 2221 to elastically stretch and permit the guide rails 2229a, 2228b, and 2229c to be in simultaneous contact with the guide rail slots 2129a, 2128b, and 2129c, respectively. In at least one embodiment, each staple driver 2220 is comprised of two or materials. For instance, in at least one such embodiment, the pillars 2220a, 2220b, and 2220c of the staple driver 2220 are comprised of a stiff plastic material, such as a glass fiber-reinforced material plastic material, for example, and the connectors 2221 are comprised of a less stiff, or more flexible, plastic material. In at least one instance, the more flexible material comprising the connectors 2221 does not have glass fiber-reinforcement or has less glass-fiber reinforcement than the pillars 2220a, 2220b, and 2220c.

Further to the above, referring again to FIGS. 27 and 28, the proximal rails 2228a of the staple drivers 2220 are not in contact with the proximal ends of the proximal rail slots 2128a of the cartridge body 2100. Similarly, the distal rails 2229b of the staple drivers 2220 are not in contact with the distal ends of the distal rail slots 2129b of the cartridge body 2220. Also, similarly, the proximal rails 2228c of the staple drivers 2220 are not in contact with the proximal ends of the proximal rail slots 2128c of the cartridge body 2110. As a result of this arrangement, a clearance gap is present between the rails 2228a, 2229b, and 2228c and the ends of the rail slots 2128a, 2129b, and 2128c, respectively. As such, the rails 2228a, 2229b, and 2228c and the ends of the rail slots 2128a, 2129b, and 2128c do not comprise control surfaces which are part of the bearing zone 2119.

Notably, further to the above, the bearing zone 2119 does not extend the entire longitudinal length of the staple driver 2220. By way of comparison, referring to FIG. 30 which depicts a cartridge body 2100′, the staple cavities of the cartridge body 2100′, including the staple cavities 2120b′, define a bearing zone 2119′ (represented by a cross-hatched area) between each staple driver 2220′ and the cartridge body 2210′ that extends along the entire length of the staple driver 2220′. The bearing zone 2119 is compact as compared to the bearing zone 2119′. In various embodiments, the longitudinal length of the bearing zone 2119 is less than half of the longitudinal length of the staple driver 2220. In other embodiments, the longitudinal length of the bearing zone 2119 is less than one third of the longitudinal length of the staple driver 2220. In at least one embodiment, the longitudinal length of the bearing zone 2119 is less than one quarter of the longitudinal length of the staple driver 2220. In at least one embodiment, the longitudinal length of the bearing zone 2119 is less than one fifth of the longitudinal length of the staple driver 2220. Given the shorter longitudinal length of the bearing zone 2119 as compared to the bearing zone 2119′, the force needed to fire the staple drivers 2220 may be less than the force needed to fire the staple drivers 2220′ thereby resulting in less force needed to push the sled 2230 distally during the staple firing stroke.

As discussed above, referring again to FIG. 26, the staple drivers 2220 ride up the ramps 2232 of the sled 2230 within the staple cavities 2120 as the sled 2230 passes thereunder. More specifically, as also discussed above, the ramp surfaces 2222 of the staple drivers 2220 slide up the ramps 2232 of the sled 2230 until the staple drivers 2220 reach the tops of the ramps 2232. At such point, as also discussed above, the staples 2240 being deformed by the staple drivers 2220 are deformed to their fully-deformed configurations. In various instances, however, a staple driver 2220 may rotate distally, or roll forward, when the sled 2230 contacts the staple driver 2220 during the staple firing stroke. In such instances, the proximal end of the staple driver 2220 may lift above the distal end of the staple driver 2220. Such rotation, or rolling, of the staple driver 2220 can increase the force needed to lift the staple driver 2220. The contact interfaces between the guide rails 2229a, 2228b, and 2229c and the guide rail slots 2129a, 2128b, and 2129c, respectively, as discussed above, can reduce, if not prevent, the rolling of the staple drivers 2220. In such instances, the guide rails 2229a, 2228b, and 2229c can co-operatively grip the cartridge body 2100 to resist the rolling of the staple drivers 2220. As the sled 2230 passes by a staple driver 2220 that has just been lifted to its fully-fired position, the staple driver 2220 becomes unsupported by the sled 2230. In such instances, absent more, the staple driver 2220 can rotate proximally, or rock back, as the sled 2230 loses contact with the staple driver 2220. Stated another way, the proximal end of the staple driver 2220 can fall below the distal end. As above, the contact interfaces between the guide rails 2229a, 2228b, and 2229c and the guide rail slots 2129a, 2128b, and 2129c, respectively, can reduce, if not prevent, the rocking of the staple drivers 2220.

Referring again to FIG. 26, each ramp 2232 of the sled 2230 comprises a first, or distal, ramp portion 2232d extending at a first angle and a second, or proximal, ramp portion 2232p extending at a second angle that is different than the first angle. The first angle is steeper than the second angle when measured from a plane B defined by the bottom of the sled 2230. As a result, the staple driver 2220 is lifted quickly on the first ramp portion 2232d and then slowly, or slower, on the second ramp portion 2232p-assuming that the longitudinal translational speed of the sled 2230 remains constant. The staples 2240 are not in contact with the anvil 3320 while the staple drivers 2220 are being lifted by the distal ramp portions 2232d and, as a result, the staple drivers 2230 do not experience high loads while being lifted quickly by the distal ramp portions 2232d. The staples 2240 come into contact with the anvil 3320 while being lifted by the proximal ramp portions 2232p which creates higher firing loads in the staple drivers 2230. The slower lifting rate of the staple drivers 2220 in such instances can be conducive to good staple formation. The ramp surfaces 2222 of the staple drivers 2220 are defined by an angle that matches the second angle of the ramps 2232. As a result, the ramp surfaces 2222 sit flush, or at least substantially flush, on the second ramp portions 2232p of the sled ramps 2232 where the staple drivers 2220 experience higher firing loads. Correspondingly, the sled ramps 2232 do not sit flush on the first ramp portions 2232d of the sled ramps 2232 where the staple drivers 2220 experience lower firing loads. The above being said, other embodiments are envisioned in which the sled ramps 2232 are defined by a constant angle and the ramp surfaces 2222 on the staple drivers 2220 extend at the same angle as the sled ramps 2232. In various embodiments, the sled ramps 2232 are defined by one or more arcuate surfaces.

The above being said, sled ramps having shallow ramp angles and sled ramps having steep ramp angles have both advantages and disadvantages. As discussed above, sled ramps having shallow ramp angles may lift the staple drivers more slowly which can result in better staple formation as compared to sled rails having steeper ramp angles. That said, sled ramps having shallow ramp angles will be longitudinally longer than sled ramps having steeper ramp angles which can require longer end effectors of the stapling instrument to house the sled. Stapling instruments having shorter end effectors may be better suited to fit within smaller spaces within the patient than longer end effectors in various instances. Correspondingly, sled ramps having steeper ramp angles may lift staple drivers more quickly and may require shorter end effectors of the stapling instruments; however, such steeper ramp angles may more readily induce forward rolling in the staple drivers during the staple firing stroke. Accordingly, finding a balance between these considerations can produce desirable results and various embodiments discussed below comprise improvements that can be used to offset one or more of these issues.

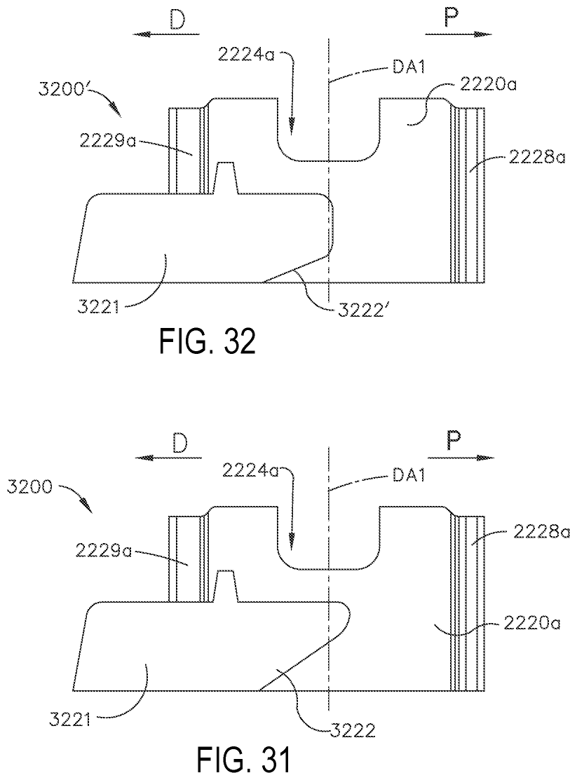

When the staple driver 2220 is lifted upwardly by the sled 2230, further to the above, the first driver pillar 2220a is driven along a first vertical drive axis, the second drive pillar 2220b is driven along a second vertical drive axis, and the third drive pillar 2220c is driven along a third vertical drive axis. Turning now to FIG. 31, a staple driver 3200 comprises a first drive pillar 2220a, a second drive pillar 2220b, and a third drive pillar 2220c in the same arrangement as the staple driver 2220. Notably, though, only the first drive pillar 2220a is depicted in FIG. 31. Also, notably, a first vertical drive axis DA1 extends through the middle, or center, of the first drive pillar 2220a and the cradle 2224a defined on the top thereof. The staple driver 3200 further comprises two connectors 3221—one connector 3221 that connects the first driver pillar 2220a and the second drive pillar 2220b and one connector 3221 that connects the second drive pillar 2220b and the third drive pillar 2220c. Each connector 3221 comprises a proximally-facing ramp surface 3222 that is configured to be engaged by a ramp of a sled during a staple firing stroke. By way of comparison, referring to FIG. 32, a staple driver 3200′ is the same as the staple driver 3200 except for the ramp surfaces 3222′ on the connectors 3221. Among other differences, the ramp surfaces 3222′ have a shallower angle—which correspond to the shallower ramps of a sled—than the ramp surfaces 3222 which correspond the steeper ramps of a different sled. Further to the above, the steeper ramp surfaces 3222 of the staple driver 3220 may, absent more, tend to cause more forward roll than the shallower ramp surfaces 3222′ of the staple driver 3220′. To address this issue, and reduce the potential forward roll of the staple driver 3220, the ramp surfaces 3222 of the staple driver 3220 extend further proximally than the ramp surfaces 3222′. As illustrated in FIG. 31, the ramp surfaces 3222 extend proximally relative to the first drive axis DA1. Moreover, although not illustrated, the third drive pillar 2220c of the staple driver 3220 is aligned laterally with the first drive pillar 2220a and the third drive axis DA3 is aligned laterally with the first drive axis DA1. As such, in this embodiment, the ramp surfaces 3222 also extend proximally relative to third drive axis DA3.

Referring again to FIGS. 23 and 23A, the staple cartridge 2000 comprises a cartridge circuit 2318 that is placed in communication with a channel jaw circuit 3318 of the cartridge jaw 3310 when the staple cartridge 2000 is seated in the cartridge jaw 3310. The cartridge circuit 2318 comprises contacts 2319 that engage contacts 3319 of the channel jaw circuit 3318 in such instances.

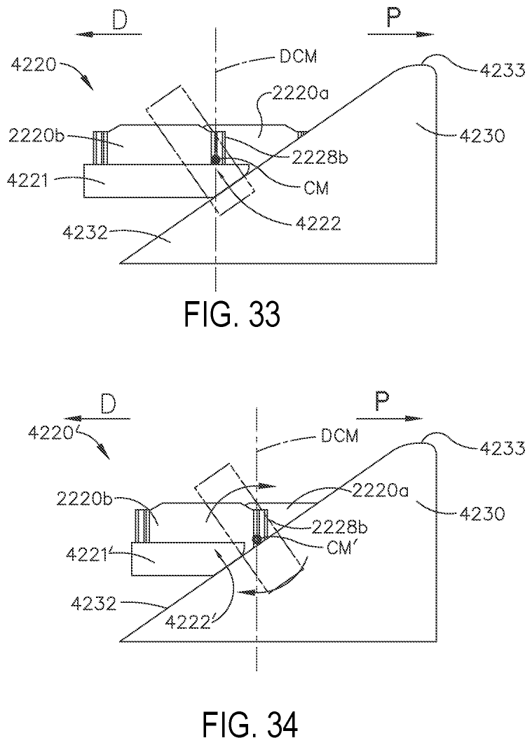

Referring now to FIG. 33, a sled 4230 is depicted lifting a staple driver 4220 during a staple firing stroke. Similar to the sled 2230, the sled 4230 comprises ramps 4232 that are configured to engage two ramp surfaces 4222 defined on the staple driver 4220. Each of the sled ramps 4232 comprises an apex 4323 which represents the fully-fired position of the staple driver 4220. Similar to the staple driver 2220, the staple driver 4220 comprises a first drive pillar 2220a and a second drive pillar 2220b and, although not illustrated, a third drive pillar 2220c arranged in the same manner as the staple driver 2220. The staple driver 4220 further comprises two connectors 4221—each having one of the ramp surfaces 4222. Also similar to the staple driver 2220, one connector 4221 connects the first drive pillar 2220a and the second drive pillar 2220b and the other connector 4221 connects the second drive pillar 2220b and the third drive pillar 2220c. Notably, the connectors 4221 and the ramp surfaces 4222 of the staple driver 4220 extend proximally relative to the center of mass CM of the staple driver 4220. As a result of the ramp surfaces 4222 extending at least partially proximally relative to the center of mass CM, the staple driver 4220 is less likely to roll forward when being lifted during the staple firing stroke. In at least one embodiment, the entirety of the ramp surfaces 4222 extend proximally relative to the center of mass CM. By way of comparison, referring to FIG. 34, a staple driver 4220′ comprises connectors 4221′ and ramp surfaces 4222′ that do not extend proximally with respect to the center of mass (CM′) of the staple driver 4220′. As a result, the staple driver 4220′ may tend to roll forward as compared to the staple driver 4220. Moreover, the ramp surfaces 4222 of the staple drivers 4220 at least partially extend proximally relative to the second drive pillar 4220b. In at least one such embodiment, the ramp surfaces 4222 extend at least partially proximally relative to the proximal guide rail 2228b of the staple driver 4220. In at least one embodiment, the entirety of the ramp surfaces 4222 extend proximally relative to the second drive pillar 4220b and/or the proximal guide rail 2228b of the staple driver 4220. Such embodiments, similar to the above, can reduce the tendency of the staple driver 4200 to roll forward while being lifted by a sled during the staple firing stroke.

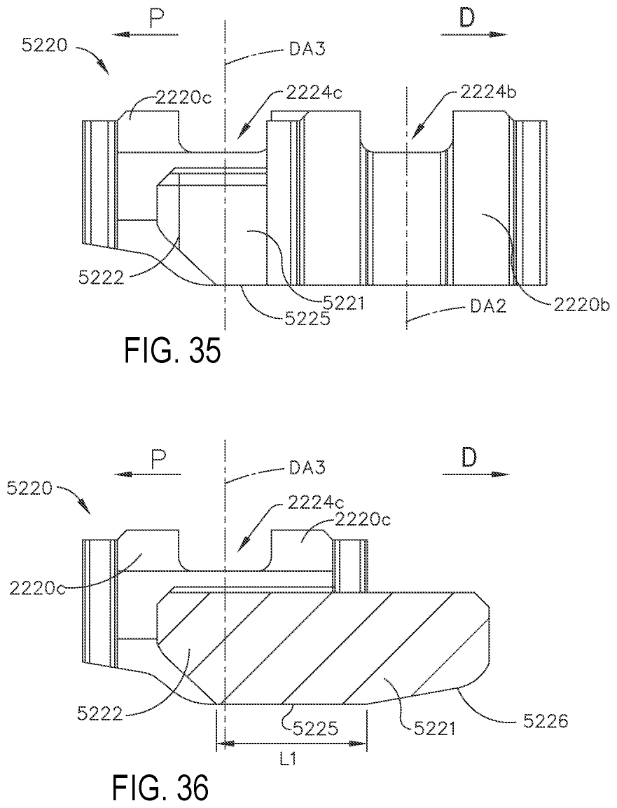

As discussed above, a staple driver may rotate proximally, or rock back, when the staple driver reaches the top, or apex, of the sled ramps. As discussed below, referring to FIGS. 35 and 36, a staple driver 5220 comprises one or more features that can prevent, or at least reduce, such rocking. Like the staple driver 2220, the staple driver 5220 comprises a first drive pillar 2220a (not illustrated), a second drive pillar 2220b, and a third drive pillar 2220c. Further to the above, the second drive pillar 2220b moves along a second drive axis DA2. Notably, the second drive axis DA2 extends through the middle, or center, of the drive pillar 2220b and the cradle 2224b defined thereon. Similarly, the third drive pillar 2220c moves along a third drive axis DA3 which extends through the middle, or center, of the third drive pillar 2220c and the cradle 2224c. Similar to the staple driver 2220, the staple driver 5220 comprises two connectors 5221—one connector 5221 that connects the first drive pillar 2220a and the second drive pillar 2220b and one connector 5221 that connects the second drive pillar 2220b and the third drive pillar 2220c. Each connector 5221 comprises a proximal-facing ramp surface 5222 that is engaged by a sled ramp during a staple firing stroke to lift the staple driver 5220 upwardly within a staple cavity 2120. Each connector 5221 further comprises a driver hold surface 5225 extending distally from the ramp surface 5222 and a distal-facing fall-off ramp 5226 extending distally from the driver hold surface 5225. The drive hold surface 5225 comprises a flat surface, but can comprise any suitable configuration. Once the sled rail has lifted the staple driver 5220 to its fully-fired position, the apex of the sled rail slides along the flat 5225 which holds the staples 2240 already deformed by the staple driver 5220 against the anvil 3320. As the sled continues further distally during the staple firing stroke, the apex of the sled rail passes under the fall-off ramp 5226 of the staple driver 5220. In such instances, the staple driver 5220 can move downwardly to relieve the pressure on the stapled tissue.

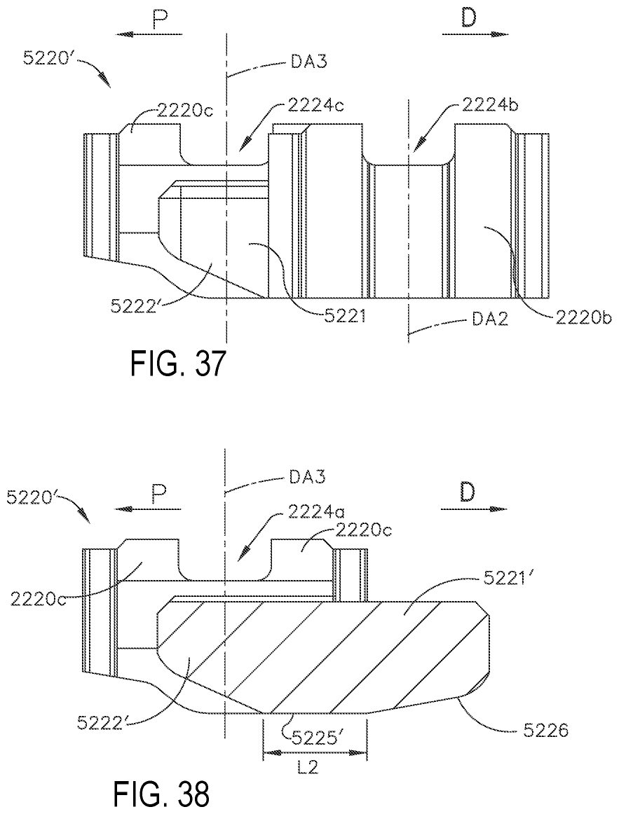

In various instances, further to the above, the staple driver 5220 can, absent more, rock backward, or proximally, when the sled rail is engaged with the flat 5225 of the staple driver 5220. When a staple driver rocks back, in certain instances, all of the staples being deformed by a staple driver may not be deformed to a desired formed height if the staple driver rocks backward. To reduce the possibility of such rocking, or the amount of such rocking, the length L1 of the flat 5225, and the position of the flat 5225, are such that the staple driver 5220 has a stable interface with the apex of the sled ramps. Notably, referring to FIG. 36, the flat 5225 extends proximally relative to the third drive axis DA3 of the third drive pillar 2220c of the staple driver 5220. Although not illustrated, the first drive pillar 2220a of the staple driver 5220 is aligned laterally with the third drive pillar 2220c such that the first drive axis DA1 of the first drive pillar 2220a is aligned laterally with the third drive axis DA3. Thus, the flat 5225 of the staple driver 5220 extends proximally relative to the first drive axis DA1 as well. Owing to this arrangement, the staple driver 5220 is less likely to rock backward during a staple firing stroke. By way of comparison, referring to FIGS. 37 and 38, a flat 5225′ defined on a connector 5221′ of a staple driver 5220′ has a length L2 that is shorter than the flat 5225 of the staple driver 5220 and does not extend proximally relative to the first drive axis DA1 and third drive axis DA3 of the first and third drive pillars 2220a and 2220c, respectively.

In various embodiments, further to the above, a sled, or at least a portion of a sled, can be retracted proximally after a staple firing stroke. In such instances, the sled may come into contact with the staple drivers 5220 as the sled is being retracted, especially if the staple drivers 5220 have fallen down within their staple cavities behind the sled after the sled has passed thereby during the staple firing stroke. The distal-facing ramps 5226 can assist in re-lifting the staple drivers 5220 into their fired positions as the sled is retracted proximally.

In many instances, surgical stapling end effectors having jaws configured to clamp tissue therebetween can experience various loads within the surgical stapling end effector. Such loads can include bending loads, shear loads, and/or torsional loads, for example. These loads can be induced when the jaws are clamped onto patient tissue and/or during a staple firing stroke, for example. In various instances, these loads can cause certain components of the surgical stapling end effectors to elastically deflect and/or twist, for example, from their original shape. In various instances, replaceable staple cartridges configured to be installed in a cartridge channel jaw of a surgical stapling end effector are particularly vulnerable, or susceptible, to such loads and the resulting deflection and/or twisting. This vulnerability can be attributed to the material of the staple cartridge relative to the materials of the cartridge channel jaw and the opposing jaw such as, for example, an anvil jaw. In at least one such instance, the staple cartridge includes a cartridge body comprised of plastic while the cartridge channel jaw and the anvil jaw are comprised metal and, owing to differences in the elasticity, flexibility, and/or strength of such materials, the staple cartridge may be more likely to elastically deflect, and/or twist, from its original shape under certain loads. Moreover, this vulnerability can also be attributed to the nature of how the components of a surgical stapling end effector fit together. In various instances, a surgical stapling end effector includes a longitudinally translatable cutting edge, or knife, that traverses a longitudinal slot defined in the staple cartridge to cut tissue clamped between the jaws. Depending on the configuration of the longitudinal slot, the longitudinal slot can reduce the strength of the staple cartridge and increase how much the staple cartridge, and/or portions of the staple cartridge, can twist or deflect from its original shape under load. In various instances, a surgical stapling end effector includes a translatable sled comprising ramped wedges configured to eject staples from the staple cartridge during the staple firing stroke. In at least one instance, the sled comprises a base portion that requires space to translate through the stapling end effector. This space, often times located between the cartridge body and a cartridge pan attached to the cartridge body, for example, or, in other instances between a cartridge body and the cartridge channel jaw where a cartridge pan is not present, provides a void into which portions of the staple cartridge can deflect.

For at least the reasons discussed above, staple cartridges can be prone to collapsing. In various instances, a staple cartridge collapses when the two lateral sides of the staple cartridge-one on each side of the longitudinal slot-torque, deflect, and/or bend inwardly toward the longitudinal slot. Such deflection of the lateral sides can cause the staple cavities defined in the lateral sides to become unregistered with, or misaligned with respect to, the corresponding staple forming pockets defined in the anvil. Such deflection can also cause binding between the various components of the end effector such as, for example, the staple drivers and the staple cavities, the I-beam and the longitudinal slot of the staple cartridge, and the sled and the cartridge body, among others. Such binding can increase the force required to staple and cut the patient tissue and even prevent the staple firing stroke from being completed. Such deflection may also cause tissue to bunch up near the longitudinal slot which can increase the difficultly of cutting the tissue during the staple firing stroke.

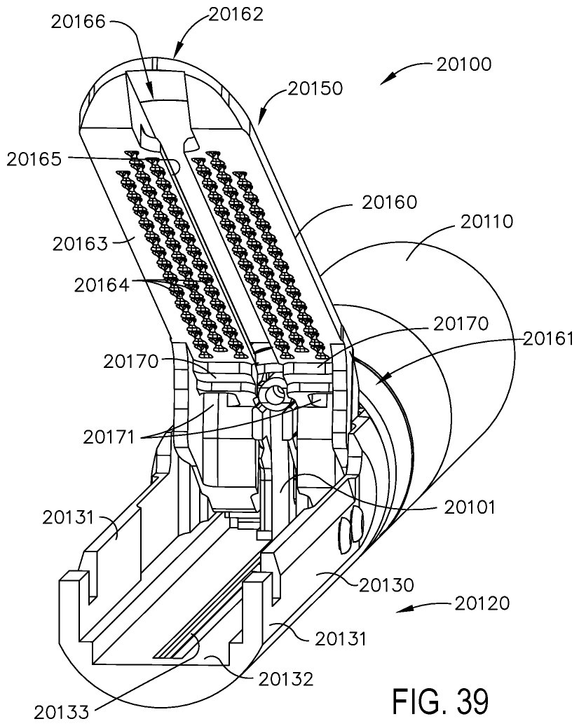

FIGS. 39-41 depict a surgical stapling end effector 20100 configured to clamp, cut, and staple patient tissue. As discussed in greater detail further below, the surgical stapling end effector 20100 is configured to inhibit the deflection of a staple cartridge. The surgical stapling end effector 20100 comprises a shaft assembly 20110, a first jaw 20120, and a second jaw 20130 pivotable relative to the first jaw 20120 to clamp tissue therebetween. The first jaw 20120 and the second jaw 20130 are supported within the shaft assembly 20110. The first jaw 20120 is non-pivotable; however, in at least one instance, the first jaw 20120 is pivotable instead of the second jaw 20130. In at least one instance, the first jaw 20120 and the second jaw 20130 are both pivotable relative to each other. Details of various surgical stapling assemblies, components, and systems can be seen in U.S. Patent Application Publication No. 2021/0059672, which is hereby incorporated by reference in its entirety herein.

The first jaw 20130 comprises a cartridge channel 20130 and a staple cartridge 20140. The staple cartridge 20140 is replaceable and can be removed and replaced with a new staple cartridge during a surgical procedure. The cartridge channel 20130 comprises a bottom portion 20132 and channel walls 20131 extending upwardly from the bottom portion 20132. The bottom portion 20132 comprises a slot 20133 defined therein configured to receive at least a portion of a distal head portion 20101 of a firing driver during a firing stroke (such as a camming pin of a distal I-beam head, for example). The firing driver, as discussed herein, can comprise any suitable component or combination of components. For example, the firing driver can comprise a motor, a rod, a firing shaft, any firing drive component, a distal I-beam head or E-beam head, a firing driver, a cutting edge, a cartridge sled, and/or staple drivers.

The second jaw 20150 is pivotable relative to the first jaw 20130 by way of a pin, for example. The second jaw 20150 comprises an anvil 20160 configured to form the staples 20102 ejected from the staple cartridge 20140. The anvil 20160 comprises a proximal end 20161, a distal end 20162, and a cartridge-facing anvil surface 20163. Tissue is configured to be clamped between the cartridge-facing anvil surface 20163 and the staple cartridge 20140 when the anvil 20160 is moved into a clamped position. Moreover, the anvil 20160 can be moved toward the staple cartridge 20140 during the firing stroke by the distal head portion 20101 which can include, for example, an I-beam. The anvil 20160 further comprises a plurality of staple forming pockets 20164 defined in the cartridge-facing anvil surface 20163 and a slot 20165 through which at least a portion of the distal head portion 20101 is configured to be received. The slot 20165 comprises an open proximal end an open distal end 20166; however, the distal end 20166 can be closed in other embodiments.

The staple cartridge 20140 comprises a cartridge body 20141 comprising two sides 20142 defined by a longitudinal slot 20144 defined in the cartridge body 20141. The longitudinal slot 20144 is configured to receive at least a portion of the distal head portion 20101 during a firing stroke. The longitudinal slot 20144 extends from a proximal end 20121 of the staple cartridge 20140 toward a distal end of the staple cartridge 20140. The longitudinal slot 20144 comprises an open end at the proximal end 20121 of the staple cartridge 20140 and a closed end at the distal end of the staple cartridge 20140. The cartridge body 20141 further comprises a deck surface 20143 and a plurality of staple cavities 20145 defined in the deck surface 20143. The staple cavities 20145 are arranged in a plurality of longitudinal rows. Each staple cavity 20145 is configured to store a staple 20102 therein. The staples 20102 are configured to be sequentially ejected from the staple cavities 20145 during the firing stroke as the distal head portion 20101 moves from a proximal, unfired position to a fired position that is distal to the proximal, unfired position.

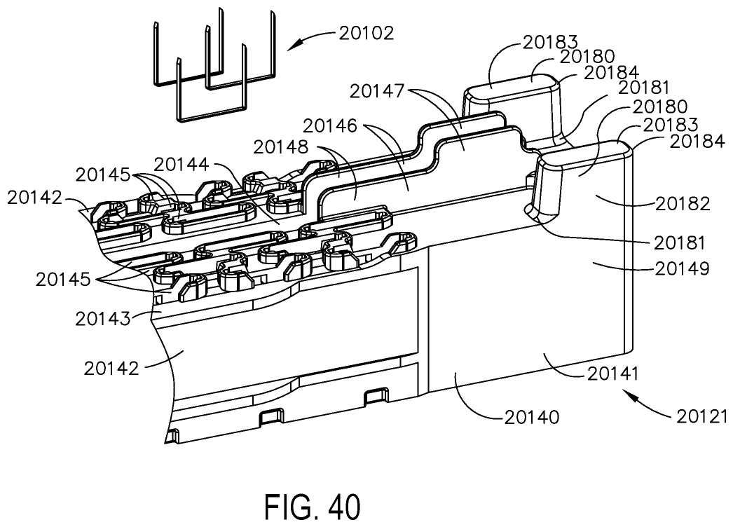

Referring primarily to FIG. 40, each side 20142 of the cartridge body 20141 further comprises a knife guard 20146 extending upwardly from the deck at the proximal end 20121 of the cartridge body 20141 adjacent the longitudinal slot 20144. Each knife guard 20146 comprises a proximal portion 20147 and a distal portion 20146. The proximal portion 20147 comprises a first longitudinal length and a first vertical height. The distal portion 20148 comprises a second longitudinal length and second vertical height. The first longitudinal length is shorter than the second longitudinal length and the first vertical height is greater than the second vertical height; however, the knife guards can have any suitable height and/or width. Moreover, other embodiments are envisioned in which the cartridge body 20141 does not have knife guards.

Each side 20142 of the cartridge body 20141 further comprises a projection 20180 extending toward the second jaw 20160 from the deck surface 20143 at the proximal end 20121 of the staple cartridge 20140. Discussed in greater detail below, the projections 20180 are configured to be received within corresponding cavities 20171 of support structures 20170 of the anvil 20160 when the end effector 20100 is in a clamped configuration. The projections 20180 are integrally formed with the cartridge body 20141. In at least one instance, the cartridge body 20141 is formed with the projections 20180 during an injection molding process. In at least one instance, the projections 20180 comprise a material that is different than the cartridge body 20141 and are attached to the cartridge body 20141 after the cartridge body 20141 is formed during an injection molding process. In at least one instance, the projections 20180 comprise a material that is different than the cartridge body 20141 and are integrally formed with the cartridge body 20141 during an insert molding process, for example. The projections 20180 comprise an elongate shape, but can comprise any suitable shape. Each projection 20180 comprises an outer wall 20182 that is flush with an outer wall 20149 of a cartridge body side 20142 of the cartridge body 20141. Each projection 20180 further comprises a filleted transition surface 20181 extending from the deck surface 20143 and a filleted transition surface 20184 extending from a top surface 20183 of the projection 20180. In at least one instance, the filleted transition surfaces 20184 aid in aligning, or guiding, the projections 20180 into the cavities 20171 defined in the anvil 20160 when the end effector 20100 is placed in a clamped configuration.

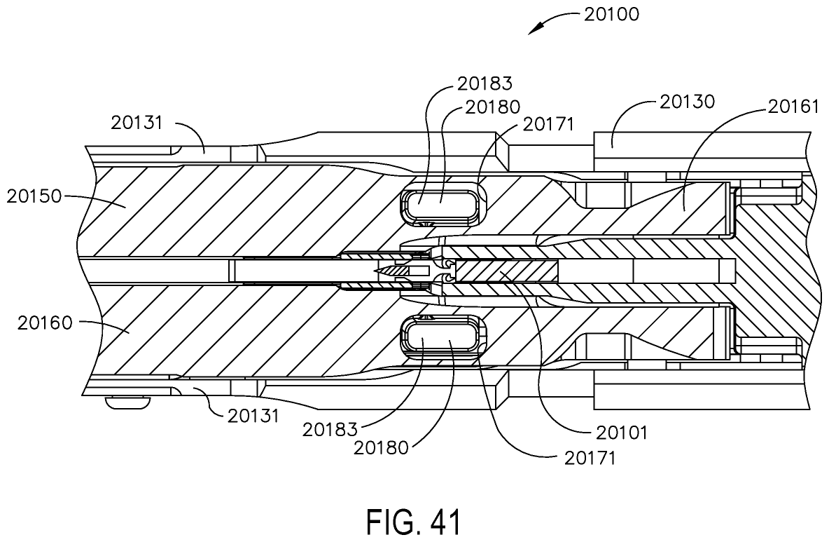

Referring to FIG. 39, the support structures 20170 of the anvil 20160 extend downwardly toward the deck surface 20143 of the cartridge body 20141 at the proximal end 20161 of the anvil 20160. Each cavity 20171 defined in each support structure 20170 is configured to receive one of the projections 20180 as the second jaw 20150 is moved into a clamped position (FIG. 41). The projections 20180 are circumferentially surrounded by the support structures 20170 when the projections 20180 are positioned within the cavities 20171. The projections 20180 are sized and configured such that they are closely received within the cavities 20171. When the staple cartridge 20140 experiences clamping and/or firing loads, for example, one or both of the projections 20810 can come into contact with the sidewalls of the cavities 20171 which resist and/or stop the deflection of the staple cartridge 20410, In various instances, as a result, such a configuration can help prevent longitudinal, lateral, and vertical deflection of the staple cartridge 20140 when the staple cartridge 20140 experiences clamping and/or firing loads, for example.

In at least one instance, the cartridge body 20141 can be more susceptible to deflection, for example, nearer the proximal end 20121 of the cartridge body 20141 owing to the longitudinal slot 20144 separating the sides of the cartridge body 20141. Thus, positioning the projections 20180 near the proximal end of the surgical stapling end effector 20100 can help reduce the deflection of the cartridge body 20141 near the proximal end 20121. In at least one instance, the top surfaces 20183 of the projections 20810 help reduce vertical deflection of the cartridge body 20141 by abutting against a corresponding surface of the support structure 20170. In at least one instance, each cavity 20171 and its surrounding cavity walls are configured to help reduce lateral and/or longitudinal deflection of the cartridge body 20141.

In various instances, the projections 20180 are press-fit into the cavities 20171 when the second jaw 20150 is moved into a fully-clamped position. In at least one instance, the projections 20180 come into contact with the walls of the cavities 20171 when the second jaw 20150 is closed. In such instances, the walls of the cavities 20171 provide immediate deflection support to the cartridge body 20141 once the second jaw 20150 is closed. In other instances, the projections 20180 do not contact cavity walls of the cavities 20171 when the second jaw 20150 is moved into a fully-clamped position; however, in such instances, the projections 20180 can come into contact with cavity walls of the cavities 20171 when the cartridge body 20141 deflects causing the projections 20180 to engage the cavity walls of the cavities 20171 which supports the cartridge body 20141. In at least one instance, the projections 20180 comprise ramped or angled front walls configured to provide a lead in surface to accommodate the pivoting motion of the second jaw 20150 into the fully clamped position. In at least one instance, the cavities 20171 comprise a ramped front cavity wall corresponding to a ramped front wall of the projections 20180.

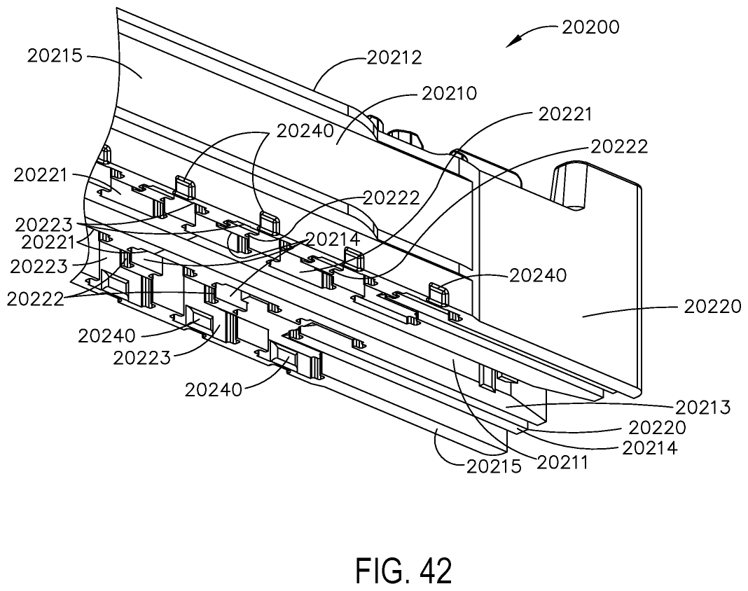

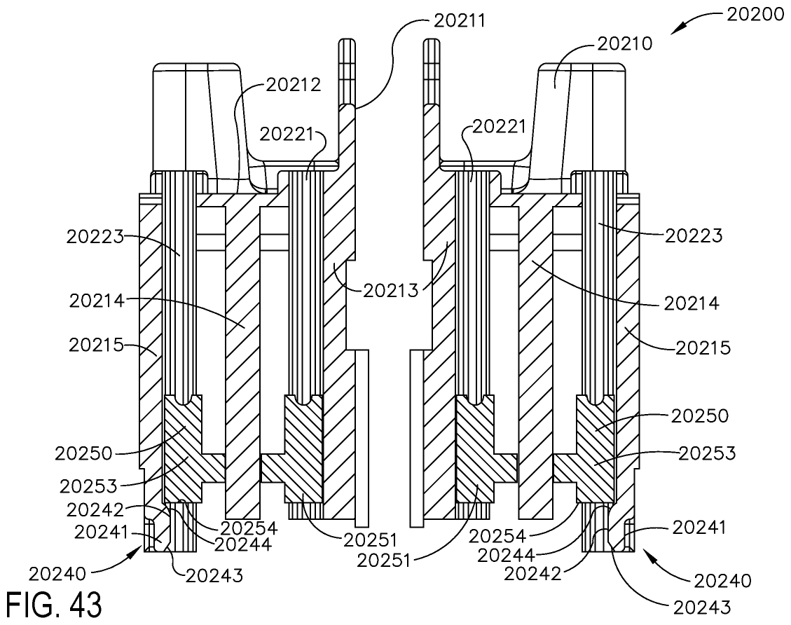

FIGS. 42 and 43 depict a staple cartridge 20200 configured to be installed in a cartridge channel of a surgical stapling end effector. The staple cartridge 20200 comprises a cartridge body 20210 and a plurality of staple drivers 20250 configured to eject staples from the staple cartridge 20200, as discussed further below. The cartridge body 20210 comprises a longitudinal slot 20211 defined by walls 20213, two sides 20220 defined by the longitudinal slot 20211, and a deck surface 20212. Each side 20220 comprises a plurality of staple cavities arranged in longitudinal rows. Each staple cavity is configured to removably store a staple therein. The staple cavities comprise inner staple cavities 20221, intermediate staple cavities 20222, and outer staple cavities 20223. The inner staple cavities 20221 are adjacent the longitudinal slot 20211. The intermediate staple cavities 20222 are positioned between the outer staple cavities 20223 and the inner staple cavities 20221. The outer staple cavities 20223 are positioned adjacent an outer cartridge wall 20215 of the cartridge body 20210. The cartridge body 20210 further comprises intermediate cartridge walls 20214 that extend longitudinally adjacent the intermediate staple cavities 20222.

Each staple driver 20250 is configured to eject three staples from the staple cavities when the staple driver 20250 is lifted vertically toward the deck surface 20212 from an unfired position (FIG. 43)—one staple from an inner staple cavity 20221, one staple from an intermediate staple cavity 20222, and one staple from an outer staple cavity 20223. Other embodiments are envisioned, however, in which a staple driver is configured to eject less than three staples or more than three staples. To help prevent the staple drivers 20250 from falling out of the bottom of the cartridge body 20210, the cartridge body 20210 further comprises retention features 20240 defined in the outer cartridge walls 20215. The retention features 20240 extend into each outer staple cavity 20223 and prevent the staple drivers 20250 from falling out of the bottom of the cartridge body 20210 from their unfired position (FIG. 43). Each staple driver 20250 comprises an inner support column 20251 positioned within an inner staple cavity 20221, an intermediate support column position within an intermediate staple cavity 20222, and an outer support column 20253 positioned within an outer staple cavity 20223. The outer support column 20253 comprises a bottom surface 20254 configured to rest on a retention feature 20140, as discussed in greater detail below.

Each retention feature 20240 comprises an inwardly facing shelf 20241. The shelf 20241 comprises a bottom lead-in edge 20243, a vertical surface 20242 extending from the lead-in edge 20243, and a shelf ledge surface 20244 extending from the vertical surface 20242. The shelf ledge surface 20244 is configured to prevent a corresponding staple driver 20250 from falling out of the bottom of the cartridge body 20210 from its unfired position (FIG. 43). In at least one instance, the bottom surface 20254 is configured to rest on the shelf ledge surface 20244 when the staple driver 20250 is in its unfired position.

The bottom lead-in edge 20243 is configured to permit insertion of the staple driver 20250 into the bottom of the cartridge body 20210 and into the staple cavities 20221, 20222, 20223. In at least one instance, the retention feature 20240 is configured to deflect at least enough to allow for the staple driver 20250 to be positioned above the shelf ledge surface 20244 at which point the retention feature 20240 and outer cartridge wall 20215 can elastically assume its original shape and, thus, hold the staple driver 20250 in its unfired position. In at least one instance, each retention feature 20240 comprises an asymmetric, or non-uniform, profile with a holding ledge at the top of the retention feature 20240 and a bottom lead-in edge at the bottom of the retention feature 20240.

In at least one instance, the retention features 20240 allow for the staple cartridge 20200 to be reloaded and/or re-assembled, for example, in an instance where the staple drivers 20250 inadvertently fall out of the bottom of the staple cartridge 20200, for example. In at least one instance, the retention features 20140 are formed using thermoplastic staking, or heat staking, for example. In at least one such an instance, the staple drivers 20250 can be inserted into the staple cavities through the bottom of the cartridge body 20211 after forming the retention features 20140 with thermoplastic staking. In at least one instance, each of the retention features 20240 are configured to be received within a corresponding retention cavity defined in the outer support column 20253 of a staple driver 20250 which releasably holds the staple driver 20250 in its unfired position. In at least one instance, the staple drivers 20250 are snap-fit into engagement with the retention features 20140 so as to hold the staple drivers 20250 in their unfired positions.

The bottom of the cartridge body 20210 comprises the bottom of the bottom of the staple cartridge 20200. The staple cartridge 20200 does not have a cartridge pan or retainer that is attached to the cartridge body 20210 that at least partially extends around the bottom of the cartridge body 20210 to prevent the staple drivers 20250 from falling out of the bottom of the cartridge body 20210. Without such a pan, the overall height of the staple cartridge 20200 can be shorter thereby saving room in the end effector. That said, the staple cartridge 20200 could comprise a cartridge pan in addition to or in lieu of the retention features 20140.

With regard to staple cartridges not having a pan attached to the cartridge body, further to the above, a space or gap may be present between the cartridge body and the jaw channel that receives the staple cartridge. This space allows a sled, for example, to move distally between the staple cartridge and the jaw channel during a firing stroke where a base portion of the sled, for example, passes underneath the cartridge body. In such embodiments, the jaw channel comprises a supporting surface for the sled. In embodiments having a pan attached to the cartridge body, a similar space or gap may be present between the cartridge body and the pan that permits a sled to move therebetween. In such embodiments, the pan comprises a supporting surface for the sled. In either event, the space or gap between the cartridge body and the supporting surface may permit the cartridge body to deflect under load, as discussed further below.

Tuning the fit between the sled, the cartridge body, and the supporting surface can help reduce the deflection of the staple cartridge, at least in certain areas, and, in various instances, reduce the force needed to perform the staple firing stroke. The fit of the base portion within the space, or gap, can be tuned in specific areas, or zones, of the staple cartridge to help prioritize either the reduction of cartridge deflection under load (tighter fit of the base portion of the sled in the space) or lower firing forces (looser fit of the base portion of the sled in the space). In one or more areas, or zones, of the staple cartridge, it may be more advantageous to prioritize the reduction of cartridge deflection under load. In one or more other zones of the staple cartridge, it may be more advantageous to prioritize lower firing forces.

In various instances, the vertical gap distance of the space defined between the sled and the support surface is dimensioned such that the sled supports the staple cartridge and reduces the vertical and/or lateral deflection of the cartridge body under load. In at least one such instance, there is little, to no, clearance between the base portion of the sled, the cartridge body, and the support surface so that the base portion of the sled closely or tightly fits between the cartridge body and the support surface. In other instances, the vertical gap distance of the space is dimensioned so as to provide one or more other portions of the staple cartridge where the sled is less tightly fit between the cartridge body and the opposing support surface thereby reducing interference between components and reducing required to perform the staple firing stroke.

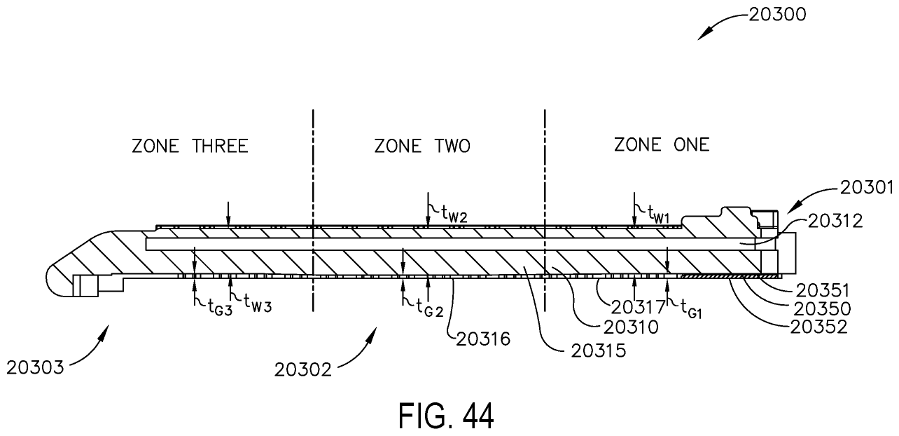

FIGS. 44-48 depict a staple cartridge assembly 20300 comprising a cartridge body 20310 including a deck surface 20311 and an inner cartridge wall 20315 adjacent a central longitudinal slot 20312 of the staple cartridge assembly 20300. The staple cartridge assembly 20300 comprises a proximal zone 20301, an intermediate zone 20302, and a distal zone 20303. In the proximal zone 20301, the inner cartridge wall 20315 does not extend all the way down to a support surface 20317 and, as a result, a gap is defined between the inner cartridge wall 20315 and the support surface 20317. Further to the above, the support surface 20317 can, for instance, be defined on a pan attached to the cartridge body 20310—in embodiments where the staple cartridge assembly 20300 has a pan—or on the jaw channel of the stapling instrument. The inner cartridge wall 20315 comprises an overall height, i.e., first vertical wall height tW1, and a first vertical gap height tG1 is defined between the inner cartridge wall 20315 and the support surface 20317. The first vertical wall thickness tW1 is defined as the distance between the deck surface 20311 and a bottom 20316 of the inner cartridge wall 20315. The first vertical gap height tG1 is defined as the distance between the bottom 20316 of the inner cartridge wall 20315 and the support surface 20317.

The staple cartridge assembly 20300 further comprises a sled 20350 actuatable by a firing actuator, for example, distally through a firing stroke to eject staples from the staple cartridge assembly 20300. The sled 20350 comprises a base portion, or support base, 20351, inner ramped wedges 20361, and outer ramped wedges 20362. The ramped wedges 20361, 20362 extend upwardly from the base portion 20351. The ramped wedges 20361, 20362 are positioned within corresponding longitudinal cavities, or slots, of the cartridge body 20310 and are configured to sequentially lift staple drivers relative to the cartridge body as the sled 20350 is moved distally during the staple firing stroke. The sled 20350 further comprises a central nose 20355 comprising a distal end 20356 that moves within the central longitudinal slot 20312. The base portion 20351 comprises inner webs 20352 between the central nose 20355 and the inner ramped wedges 20361 and, also, outer webs 20353 between the inner ramped wedges 20361 and the outer ramped wedges 20362. The inner webs 20352 and the outer webs 20353 have different vertical thicknesses; however, in other embodiments, the webs 20352 and 20353 can have the same vertical thickness. In either event, as discussed in greater detail below, a portion of the sled 20350 travels within the gap defined between the inner cartridge wall 20315 and the support surface 20317.

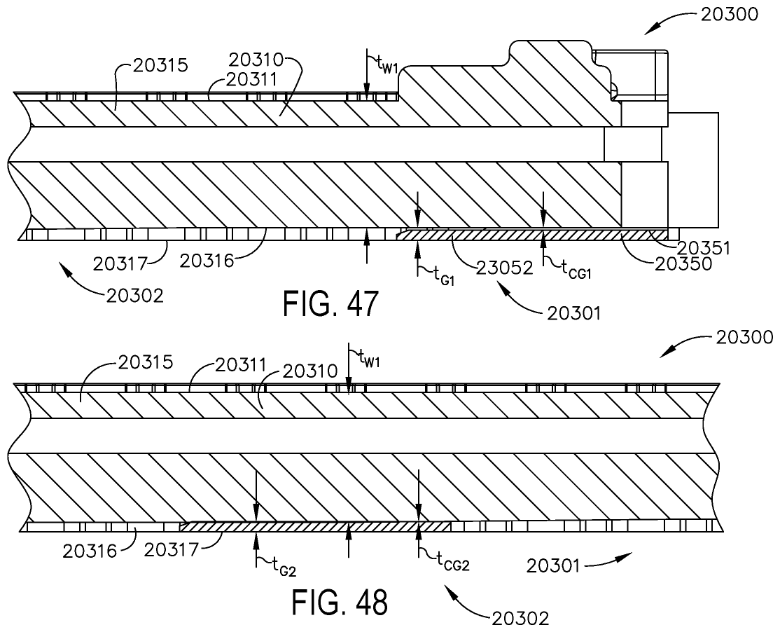

As can be seen in FIG. 47, further to the above, the inner web 20352 travels within the gap defined between the inner cartridge wall 20315 and the support surface 20317. The inner web 20352 is sized and configured such that there is a first clearance gap tCG1 between the top of the inner web 20352 and the bottom 20316 of the inner cartridge wall 20315. The first clearance gap tCG1 provides space for the inner web 20352 to pass underneath the cartridge body 20310 in the proximal zone 20301 so as to reduce contact, or frictional forces, between the sled 20350, cartridge body 20310, and/or support surface 20317. This reduction in frictional force can reduce the force required to perform the staple firing stroke in the proximal zone 20301. Such an arrangement can help ease the load on a motor of the staple firing system at the beginning of a firing stroke where an initially high firing force may be undesirable.

In the intermediate zone 20302, the inner cartridge wall 20315 comprises a second vertical wall height tW2 and a second vertical gap height tG2. The second vertical wall height tW2 is defined as the distance between the deck surface 20311 and a bottom 20316 of the inner cartridge wall 20315. The second vertical gap height tG2 is defined as the distance between the bottom 20316 of the inner cartridge wall 20315 and the support surface 20317. The second vertical wall height tW2 is greater than the first vertical wall height tW1. Thus, the second vertical gap height tG2 is less than the first vertical gap height tG1.

As can be seen in FIG. 48, the inner web 20352 travels within the second vertical gap height tG2 such that there is a second clearance gap tCG2 between the top of the inner web 20352 and the bottom 20316 of the inner cartridge wall 20315. The second clearance height tCG2 provides a small gap between the inner web 20352 and the bottom 20316 of the inner cartridge wall 20315 as the sled passes through the intermediate zone 20302. During the staple firing stroke, however, the cartridge body 20310 may deflect downwardly such that the second clearance gap tCG2 is eliminated and the inner cartridge wall 20315 is in contact with the sled 20350. In such instances, the inner web 20352 of the sled 20350 is in contact with the inner cartridge wall 20315 as the sled 20350 is advanced distally. In alternative embodiments, the second clearance height tCG2 is not present prior to the firing stroke and during the firing stroke. In any event, as the inner web 20352 passes underneath the cartridge body 20310 in the intermediate zone 20302, the inner web 20352 can provide support to the inner cartridge wall 20315 and, thus, the cartridge body 20310. Although the contact between the inner web 20352 of the sled 20350 and the inner cartridge wall 20315 increases the force needed to push the sled 20350 through the firing stroke, the reduction of cartridge body deflection is prioritized over required firing forces in the intermediate zone 20302, for example. In at least one instance, the second clearance gap tCG2 is less than the first clearance gap tCG1 but not zero. In at least one instance, the second clearance gap tCG2 is zero or at least substantially zero relative to possible manufacturing tolerances. In at least one instance, the second vertical gap height tG2 (the space through which the inner web 20352 travels) is less than the vertical height of the inner web 20352. In such an instance, the inner web 20352 would positively deflect the inner cartridge wall 20315 vertically away from the opposing support surface 20317.

In the distal zone 20303, the inner cartridge wall 20315 comprises a third vertical wall height tW3 providing a third vertical gap height tG3. The third vertical wall height tW3 is defined as the distance between the deck surface 20311 and a bottom 20316 of the inner cartridge wall 20315. The third vertical gap height tG3 is defined as the distance between the bottom 20316 of the inner cartridge wall 20315 and the support surface 20317. The second vertical wall height tW2 is greater than the first vertical wall height tW1 and the third vertical wall height tW1. Thus, the second vertical gap height tG2 is less than the first vertical gap height tG1 and the third vertical gap height tG3. The inner web 20352 moves within the third vertical gap height tG2 such that there is a third clearance gap similar to, or the same as, the first clearance gap tCG1 between the top of the inner web 20352 and the bottom 20316 of the inner cartridge wall 20315. The cartridge body 20310 may be less susceptible to deflection, and thus may need less support, near its distal end given that the two sides of the cartridge body are connected at the nose, or distal end, of the cartridge body.

In various embodiments, further to the above, each zone 20301, 20302, 20303 has a different vertical wall height and a different vertical gap height than the other. In at least one instance, the magnitude of the vertical wall height gradually increases from a proximal end of the staple cartridge assembly 20300 to a distal end of the staple cartridge assembly 20300. In another instance, the magnitude of the vertical wall height gradually decreases from a proximal end of the staple cartridge assembly 20300 to a distal end of the staple cartridge assembly 20300. In at least one instance, the magnitude of the vertical gap height gradually increases from a proximal end of the staple cartridge assembly 20300 to a distal end of the staple cartridge assembly 20300. In another instance, the magnitude of the vertical gap height gradually decreases from a proximal end of the staple cartridge assembly 20300 to a distal end of the staple cartridge assembly 20300. In any event, the magnitude of the vertical wall height and/or the vertical gap height can be specifically tuned for specific zones of the cartridge body 20310 to provide a desired balance between the deflection support that the sled 20350 can provide during the firing stroke and the firing force needed to complete the firing stroke.

As described above, the sled 20350 can interact, or at least potentially interact, with an inner cartridge wall 20315 of the cartridge body 20310. As also described above, the cartridge body 20310 comprises two inner cartridge walls 20315—one on each side of the longitudinal slot 20312 defined in the cartridge body 20310. In the embodiment depicted in FIGS. 44-48, the sled 20350 is configured to engage both of the inner cartridge walls 20315 as described above. The sled 20350 comprises two inner support webs 20352 which can each support an inner cartridge wall 20315 and support the cartridge body 20310 equally, or at least substantially equally, on both sides of the cartridge body 20310. Moreover, as discussed above, the cartridge body 20310 comprises longitudinal cartridge walls in addition to the inner cartridge walls 20315. In various embodiments, the sled 20350 can comprise one or more additional support webs that can engage and support the longitudinal cartridge walls of the cartridge body 20310.

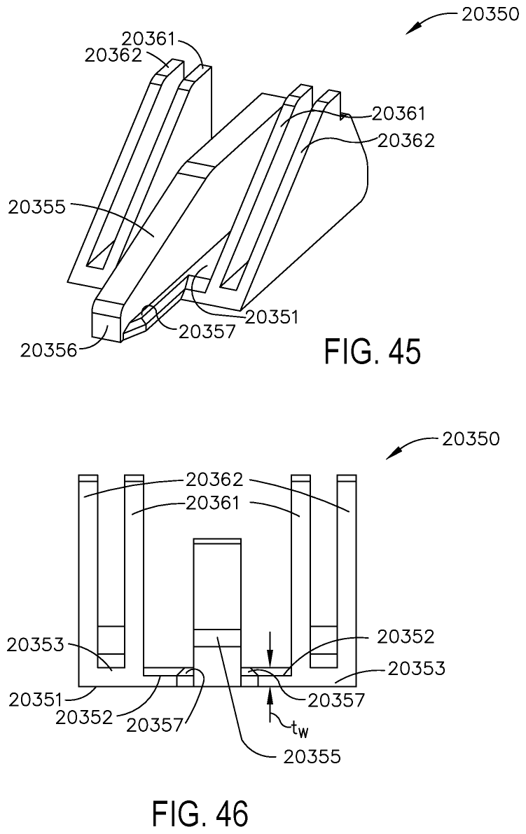

As discussed above, the inner cartridge walls 20315 can deflect when a compressive load, for example, is applied to the deck of the cartridge body 20310. As can be seen in FIGS. 45 and 46, the sled 20350 further comprises horizontal ledges 20357 extending laterally, or outwardly, from the distal end 20356 of the central nose portion 20355 that can engage and support the inner cartridge walls 20315 and inhibit the cartridge body 20310 from deflecting, or at least deflecting further. The horizontal ledges 20357 are configured to support the inner cartridge walls 20315 in at least two ways. More specifically, the horizontal ledges 20357 extend laterally under the inner cartridge walls 20315 and can support the cartridge body 20310 from deflecting downwardly and, also, extend between the inner cartridge walls 20315 and can support the inner cartridge walls 20315 from deflecting inwardly. The horizontal ledges 20357 comprise distal lead-in surfaces configured to pry the inner cartridge walls 20315 open and/or lift the inner cartridge walls 20315 upwardly. The distal lead-in surfaces can help reduce binding, or jamming, between the sled 20350 and the cartridge body 20310, especially in instances of higher clamping loads between the end effector jaws. The horizontal ledges 20357 are configured to support the inner cartridge walls 20315 at a location that is distal to, or ahead of, the ramped wedges 20361, 20362 engaging the staple drivers during the firing stroke. As a result, the horizontal ledges 20357 can help support the inner cartridge walls 20315 and reduce cartridge body 20310 deflection distally, or ahead, of a firing driver, such as an I-beam and/or tissue cutting knife, for example, moving between the inner cartridge walls 20315 during the firing stroke.

In various embodiments, further to the above, an I-beam is movable from a proximal unfired position to a distal fired position during a firing stroke to push the sled 20350 distally. The I-beam comprises a first cam that engages a first jaw of the end effector and a second cam that engages a second jaw of the end effector. In at least one embodiment, the first cam and the second cam each comprise a flange extending laterally from a central portion of the I-beam, for example. As the I-beam is moved distally during the firing stroke, the I-beam can pull the first jaw and the second jaw toward one another to compress the patient tissue positioned therebetween. In various instances, the location of the I-beam represents the location at which the staple cartridge may be most prone to collapsing.

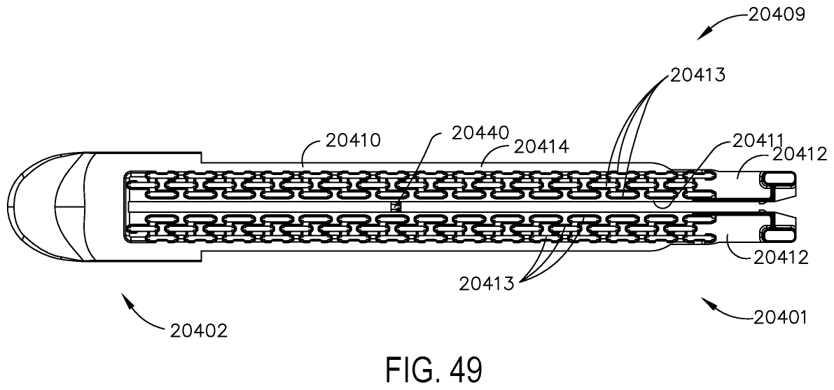

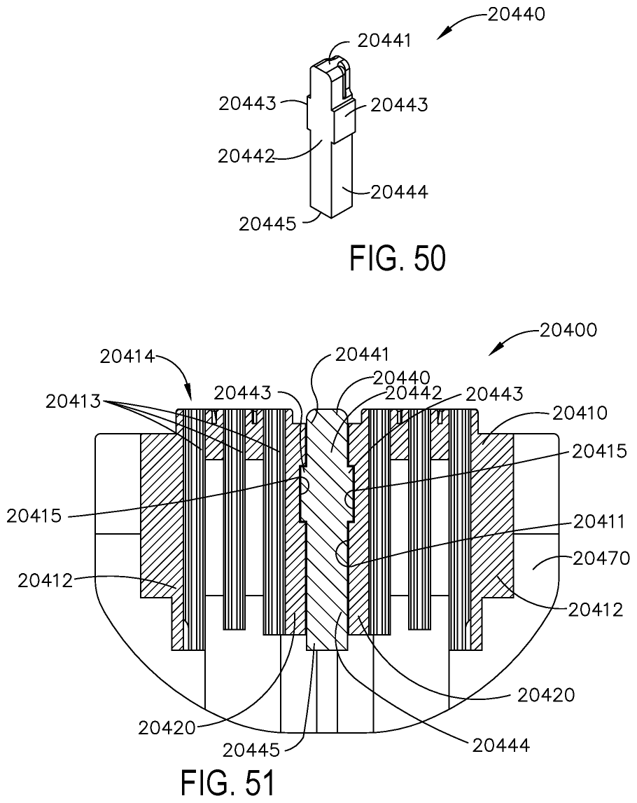

FIGS. 49-51 depict a surgical stapling assembly 20400 configured to help reduce cartridge deflection during a firing stroke. The surgical stapling assembly 20400 comprises an anvil jaw, a channel jaw 20470, a staple cartridge 20409 positioned within the channel jaw 20470, and a cartridge support pillar 20440 slideably positioned within the staple cartridge 20409. The staple cartridge 20409 comprises a proximal end 20401 and a distal end 20402. The staple cartridge 20409 comprises a cartridge body 20410 comprising two sides 20412 defined by a longitudinal slot 20411 extending therebetween that is configured to receive at least a portion of a firing driver, such as a sled, cutting edge, I-beam, and/or firing shaft, for example, during a firing stroke. Each side 20412 comprises a plurality of staple cavities 20413 defined in a deck surface 20414 of the cartridge body 20410 that are each configured to removably store a staple therein. As can be seen in FIG. 50, the cartridge support pillar 20440 comprises an upper portion 20441, an intermediate portion 20442, and a lower portion 20444. The cartridge support pillar 20440 is closely received within the longitudinal slot 20411 and is configured to slide within the longitudinal slot 20411 during firing stroke. The cartridge support pillar 20440 is configured to counter, prevent, and/or resist cartridge deflection in the cartridge body 20410 when the cartridge body 20410 is subjected to a compressive, or clamping, load. As discussed in greater detail below, the cartridge support pillar 20440 is configured to transfer or transmit loads that would otherwise cause the deflection of the cartridge body 20410.

The intermediate portion 20442 of the cartridge support pillar 20440 comprises laterally extending ledges, or wings, 20443 providing a greater width of the intermediate portion 20442 relative to the upper portion 20441 and the lower portion 20444. The wings 20433 are received within corresponding lateral slots 20415 defined in inner cartridge walls 20420 of the longitudinal slot 20411. The wings 20433 are sized and configured such that the wings 20433 are in contact with, or engaged with, the inner cartridge walls 20420 prior to the cartridge body 20410 being subjected to a compressive, or clamping, load. In other embodiments, the wings 20433 are sized and configured such that the wings 20433 are not in contact with the inner cartridge walls 20420 prior to the cartridge body 20410 being subjected to a compressive, or clamping, load; however, the inner cartridge walls 20420 can deflect into contact with the wings 20433 when the cartridge body 20410 is subjected to a compressive load. The engagement between the wings 20443 and the side walls of the lateral slots 20415 can help support the inner cartridge walls 20420 and reduce the vertical and/or lateral deflection thereof, as well as of the cartridge body 20410 overall. The cartridge support pillar 20440 can reduce or prevent collapsing within the staple cartridge 20409 when the jaws of the surgical stapling assembly 20400 are closed and/or during the staple firing stroke. In at least one respect, the cartridge support pillar 20440 provides a buffer structure within the longitudinal slot 20411 to prevent the sides 20412 from buckling the longitudinal slot 20411 inwardly. Moreover, as discussed in greater detail below, the cartridge support pillar 20440 can redirect clamping load into the channel jaw 20470 through the ledges 20443 without the clamping load flowing through the deck 20414.

In use, further to the above, patient tissue can be clamped against the deck 20414 of the cartridge body 20410 when the surgical stapling assembly 20400 is moved into a clamped configuration. Also, in use, patient tissue can be further compressed against the cartridge body 20410 during the staple firing stroke. In either instance, a compressive load flows through the cartridge body 20410 into the jaw 20470 and, as described above, the compressive load can distort the cartridge body 20410. Referring to FIG. 51, the cartridge support pillar 20440 transmits a portion of the compressive load directly into the jaw 20470 without the load passing through the cartridge body 20410, or at least without passing through the deck 20414 of the cartridge body 20410. The cartridge support pillar 20440 comprises a bottom surface 20445 configured to be supported by the channel jaw 20470—either directly or through a cartridge pan attached to the cartridge body 20410 which is in contact with the channel jaw 20470. In various instances, the upper portion 20441 of the cartridge support pillar 20440 is flush with top surface of the deck 20414 while, in other instances, the upper portion 20441 extends above the deck 20414 so as to intercept clamping forces so that the clamping forces can be redirected through the cartridge support pillar 20440 into the channel jaw 20470.

Referring now to FIG. 49, the cartridge support pillar 20440 is positioned near an intermediate zone of the staple cartridge 20409, i.e., at a location between the proximal end of the staple cartridge 20409 and a distal end of the staple cartridge 20409. In at least one instance, the cartridge support pillar 20440 is pre-positioned in the position illustrated in FIG. 49 so as to absorb clamping forces at this position ahead of a staple firing stroke. As the jaws of the end effector are clamped together to initially clamp tissue and/or as an I-beam starts to apply final clamping forces during the beginning of a firing stroke, for example, clamping loads are experienced throughout the longitudinal length of the staple cartridge 20409. Pre-positioning the cartridge support pillar 20440 in the intermediate zone, for instance, helps reduce cartridge deflection ahead of the firing stroke at the position of the cartridge support pillar 20440. In at least one instance, the cartridge support pillar 20440 is pre-positioned at the beginning of the staple firing stroke, or closer to the proximal end of the staple cartridge 20409, instead of near the intermediate zone of the staple cartridge 20409. In other instances, the cartridge support pillar 20440 is pre-positioned toward the end of the staple firing stroke, or closer to the distal end of the staple cartridge 20409. In any of these instances, as described in greater detail below, the cartridge support pillar 20440 is configured to be moved distally by the firing driver when the firing driver comes into contact with the support pillar 20440.

When the firing driver reaches the cartridge support pillar 20440, referring to FIG. 49, the firing driver pushes the cartridge support pillar 20440 toward the distal end 20402 during the rest of the staple firing stroke. As the cartridge support pillar 20440 is pushed distally by the firing driver, the cartridge support pillar 20440 dynamically helps reduce cartridge deflection as the support pillar 20440 moves distally through the rest of the firing stroke. Stated another way, it should be appreciated that the highest loads, or at least some of the highest loads, experienced in the surgical stapling assembly 20400 are located around the staples being fired, i.e., pushed upwardly toward and against the anvil jaw by the sled which is being pushed distally by, for example, an I-beam, and, when the sled and the support pillar 20440 are both pushed distally by the I-beam during the staple firing stroke, the support pillar 20440 is advantageously present at, or adjacent to, these high loads. As a result, the support pillar 20440, in such instances, can provide effective support that moves in conjunction with the occurrence of these high loads.

In various instances, a plurality of cartridge support pillars 20440 are pre-positioned within the longitudinal slot 20411. In at least one such instance, a proximal cartridge support pillar, an intermediate cartridge support pillar, and a distal cartridge support pillar are positioned in the longitudinal slot 20411 and are evenly spaced throughout a staple firing stroke, for example. In such an instance, the firing driver travels a first distance before the firing driver bumps into the proximal cartridge support pillar. The firing driver then travels a second distance before the proximal cartridge support pillar, and/or the firing driver, bumps into the intermediate cartridge support pillar. The firing driver then travels a third distance before the intermediate cartridge support pillar bumps into the distal cartridge support pillar. A final distance is traveled where the firing driver pushes all of the support pillars into a final position at the completion of the firing stroke. In at least one instance, the first distance, the second distance, the third distance, and the final distance are the same. In at least one other instance, the first distance, the second distance, the third distance, and the final distance are different. In at least one instance, a first one of the first distance, the second distance, the third distance, and the final distance is equal to a second one of the first distance, the second distance, the third distance, and the final distance but different from a third one of the first distance, the second distance, the third distance, and the final distance, for example.

In at least one instance, the number and/or size of the cartridge support pillars 20440 pre-positioned within the staple cartridge 20409 can be selected based on the length of the staple cartridge, the thickness of tissue to be cut and stapled, and/or the size of the staples in the staple cartridge. For example, where higher clamping pressures are expected with thicker tissue, additional cartridge support pillars 20440 can be utilized. In at least one instance, the staple cartridge 20409 comes with a plurality of cartridge support pillars 20440 and a user can insert the desired number of cartridge support pillars 20440 in the longitudinal slot 20411 and/or remove unwanted cartridge support pillars 20440. In at least one instance, the user can also select the desired position of each cartridge support pillar 20440. In at least one instance, the longitudinal slot 20411 comprises pre-defined alignment detents defined in the cartridge body 20410 to align and/or releasably retain the cartridge support pillars 20440 in position.

In at least one instance, the cartridge support pillar 20440 is configured to carry and deliver a hemostatic agent to tissue. In at least one instance, the hemostatic agent comprises oxygenated regenerated cellulose, for example. In at least one instance, the cartridge support pillar 20440 is coated in the hemostatic agent such that the hemostatic agent is distributed onto tissue upon rubbing against the tissue, for example. In at least one instance, the cartridge support pillar 20440 comprises a reservoir to carry the hemostatic agent. In at least one instance, the sled and/or I-beam is configured to puncture the reservoir upon bumping into the cartridge support pillar 20440 during the staple firing stroke. In such an instance, the cartridge support pillar 20440 can be press fit into the longitudinal slot 20411 and/or held with support detents extending inwardly from the walls of the longitudinal slot 20411, for example, so as to provide enough holding force to the cartridge support pillar 20440 to be punctured prior to being pushed distally. In such an instance, the hemostatic agent is applied to the tissue near the cut line for the remainder of the firing stroke. In at least one instance, only a proximal cartridge support pillar of the plurality of support pillars 20440 comprises a hemostatic agent. In at least one instance, more than one cartridge support pillar 20440 comprises a hemostatic agent configured to be delivered to tissue during a firing stroke.

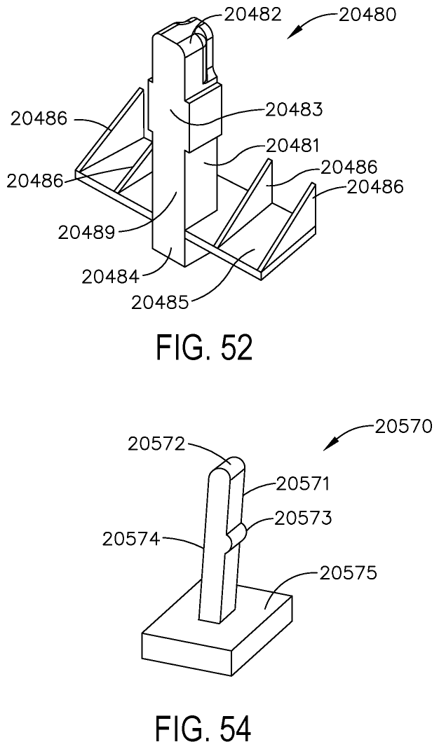

Further to the above, referring again to the embodiment of FIGS. 49-51, a firing driver, such as an I-beam, for example, pushes a sled and one or more support pillars distally during a staple firing stroke. The sled comprises ramps that engage the staple drivers of a staple cartridge to eject the staples stored therein and, in at least one embodiment, a gap or slot is defined between two of the sled ramps that is configured to receive, or at least partially receive, a support pillar therein. In at least one such embodiment, as a result, the I-beam pushes the sled and the sled pushes the support pillar during a staple firing stroke. In at least one embodiment, the support pillar comprises one or more ramps which co-operate with the ramps of the sled to lift the staple drivers and staples of a staple cartridge during the staple firing stroke. FIG. 52 depicts a cartridge support pillar 20480 comprising a primary support pillar body 20481 and a sled portion 20485 extending from the primary support pillar body 20481. In at least one embodiment, the support pillar 20480 is pre-positioned in a staple cartridge at a location that is proximal to a distal-most row of staple drivers such that, when the primary support pillar body 20481 is engaged and pushed distally by a firing driver, the sled portion 20485 at least partially lifts the distal-most row of staple drivers to ensure that the distal-most staples supported by the distal-most row of staple drivers, for instance, are formed fully during the staple firing stroke. The primary support pillar body 20481 comprises an upper portion 20482, a wider intermediate portion 20483, and a lower portion 20484. The sled portion 20485 extends from the lower portion 20484. The sled portion 20485 comprises ramped wedges 20486 configured to engage staple drivers when the cartridge support pillar 20480 is advanced distally by the firing driver. Such a configuration can ensure that the distal-most staples of each longitudinal staple row are fully formed at the completion of the staple firing stroke.

In various embodiments, further to the above, the firing driver is retracted proximally after the staple firing stroke. In at least one such embodiment, the sled and the support pillars are not retracted proximally with the firing driver after the staple firing stroke. In such embodiments, the support pillars remain at the distal end of the staple cartridge after the firing stroke has been completed. In at least one embodiment, the distal end, or nose, of the cartridge body comprises a housing to store or stow one or more cartridge support pillars therein. In circumstances where the firing driver is retracted before the staple firing stroke has been completed, in such embodiments, the sled and the support pillars are left in place at an intermediate location in the staple firing stroke.

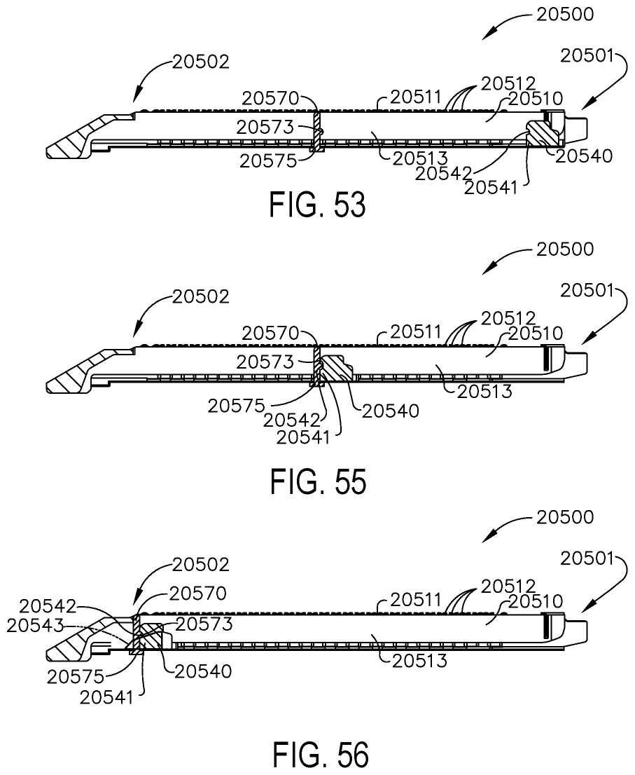

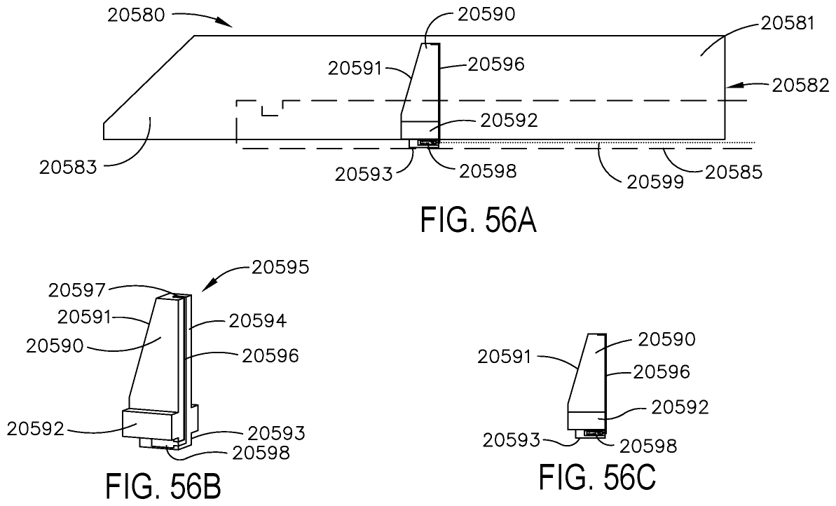

As discussed above, a sled of a staple cartridge can be configured to contact a support pillar and push the support pillar during a staple firing stroke. As discussed in greater detail further below, the sled can also be configured to extend under at least a portion of the support pillar and receive a compressive or clamping load from the support pillar. FIGS. 15-18 depict a staple cartridge assembly 20500 for use with a surgical stapling end effector such as, for example, those disclosed herein. The staple cartridge assembly 20500 comprises a cartridge body 20510, a sled 20540 configured to eject staples stored in the cartridge body 20510, and a cartridge support pillar 20570 configured to help reduce cartridge deflection. The cartridge body 20510 comprises a deck surface 20511 configured to support patient tissue thereon, a plurality of staple cavities 20512 defined in the deck surface 20511 that are configured to removably store a plurality of staples therein, and a longitudinal slot 20513 through which at least a central portion 20541 of the sled 20540 is configured to travel and through which at least a portion of a firing driver which pushes the sled 20540 distally is configured to travel. The firing driver is configured to push the sled 20540 from a proximal, unfired position (FIG. 53) to a distal, fired position (FIG. 56) to eject the staples from the staple cartridge assembly 20500. In at least one instance, the firing driver comprises the sled 20540, a firing shaft, a tissue cutting edge, and/or a distal I-beam head, for example. The staple cartridge assembly 20500 does not have a cartridge pan and the cartridge body 20510 is directly supported by the cartridge channel of a surgical stapling instrument when the staple cartridge assembly 20500 is installed in the cartridge channel. In alternative embodiments, the staple cartridge assembly 20500 comprises a cartridge pan attached to the cartridge body 20510 configured to abut the cartridge channel when the staple cartridge assembly 20500 is installed in the cartridge channel.

The cartridge support pillar 20570 is configured to help reduce deflection of the cartridge body 20510 at and/or around the location of the cartridge support pillar 20570 during the application of clamping loads to the cartridge body 20510. In at least one instance, the cartridge support pillar 20570 is pre-positioned along the firing stroke such as, for example, at the position shown in FIG. 53 such that the cartridge support pillar 20570 can support the cartridge body 20510 ahead of the firing driver and/or sled 20540 during the firing stroke. The cartridge support pillar 20570 comprises an upper pillar portion 20571 and a base portion 20575. The upper pillar portion 20571 comprises a top 20572, a protrusion 20573, and a bottom portion 20574. The base portion 20575 extends from the bottom portion 20574. The base portion 20575 is configured to be supported by a cartridge channel of a stapling instrument when the staple cartridge assembly 20500 is seated in the cartridge channel. In at least one instance, the base portion 20575 sits directly on top of a support surface of the cartridge channel opposite a bottom of the cartridge body 20510. Moreover, the base portion 20575 is positioned and configured to translate within the same space, or cavity, within which a base portion of the sled 20540 is configured to travel during the staple firing stroke.

The sled 20540 comprises a central portion 20541 comprising a notch that defines a shoulder 20542 configured to engage the protrusion 20573 when the sled 20540 contacts the cartridge support pillar 20570 during the staple firing stroke. In such instances, the shoulder 20542 extends under the protrusion 20573 such that the support pillar 20570 can sit on the shoulder 20542 of the sled 20540 when the support pillar 20570 is subjected to a compressive load. As a result, both the support pillar 20570 and the sled 20540 can both transmit the compressive load to a support structure positioned thereunder. In embodiments where the staple cartridge assembly 20500 comprises a pan attached to the cartridge body 20510, the sled 20540 can slide on top of the pan and transmit the compressive load from the support pillar 20570 to the pan. In embodiments where the staple cartridge does not comprise a pan attached to the cartridge body 20510, the sled 20540 can slide on top of a support surface defined on the cartridge channel and transmit the compressive load from the support pillar 20570 to the cartridge channel. In various embodiments, the cartridge body 20510 comprises one or more support shoulders upon which the sled 20540 slides that are configured to receive the compressive load from the sled 20540.

As can be seen in FIG. 56, further to the above, the cartridge support pillar 20570 is configured to be nested longitudinally within the sled 20540 when the sled 20540 engages the cartridge support pillar 20570. This nested longitudinal arrangement allows the cartridge support pillar 20570 to be positioned entirely proximal with respect to a distal end of the ramped wedges of the sled 20540. A ramped wedge 20543 is illustrated in phantom in FIG. 56 illustrating that the cartridge support pillar 20570 is proximal to a distal end of the ramped wedge 20543 once the sled 20540 and the support pillar 20570 have become fully nested. Such a configuration helps reduce the overall length of a firing assembly and can reduce the need for additional longitudinal space at the distal end, or nose, of the staple cartridge to accommodate the firing assembly at the end of the staple firing stroke while ensuring that all of the staples are fully fired by the sled.

FIGS. 56A-56C depict a surgical stapling system 20580 comprising a staple cartridge 20581 having a proximal end 20852 and a distal end 20853 and a cartridge channel 20585 within which the staple cartridge 20851 is positioned. Similar to other staple cartridges disclosed herein, the staple cartridge 20851 comprises a movable cartridge support 20590. The surgical stapling system 20580 further comprises a sensing system 20595 configured to detect a parameter of the surgical stapling system 20580 at the movable cartridge support 20590. Like the other movable cartridge supports disclosed herein, the movable cartridge support 20590 is pre-positioned within the staple cartridge 20581. The movable cartridge support 20590 is positioned within a longitudinal slot of the staple cartridge 20581 and is configured to be pushed distally by a firing driver, such as an I-beam, tissue cutting knife, and/or firing bar, for example, and/or a sled pushed distally by the firing driver, during a staple firing stroke. The movable cartridge support 20590 may be pre-positioned at a high load location such that the movable cartridge support 20590 is configured to reduce deflection of the staple cartridge 20581 within the high load location.

The movable cartridge support 20590 comprises an upper portion 20591 positioned within the central longitudinal slot of the staple cartridge 20851, a lateral-flange portion 20592, and a lower portion 20593. The lateral-flange portion 20592 is positioned within a longitudinal cavity defined between the bottom of the staple cartridge 20581 and an opposing support surface of the cartridge channel 20585. The lateral-flange portion 20592 is configured to fill this longitudinal cavity at the location of the movable cartridge support 20590 so as to help reduce vertical and lateral cartridge deflection by transferring the clamping load experienced by the staple cartridge 20581 to the cartridge channel 20585. The lateral-flange portion 20592 is sized and configured such that it is engaged with, or in close proximity to, the sidewalls of the longitudinal cavity. At least a portion of the lower portion 20593 is configured to slide within a longitudinal channel slot defined in the cartridge channel 20585. The longitudinal channel slot is aligned, or at least substantially aligned, with the central longitudinal slot defined in the staple cartridge 20851 such that the movable cartridge support 20590 extends through the longitudinal channel slot of the cartridge channel and the central longitudinal slot defined in the staple cartridge 20851.

In at least one instance, the movable support 20590 can be utilized as a vehicle for a load-detection system that measures the clamping load at the movable support 20590. The sensing system 20595 comprises a sensor 20597, an electrical lead 20596, and an electrical contact 20598 connected to the sensor 20597 by the electrical lead 20596. The sensor 20597 comprises any suitable sensor such as, for example, a load cell and/or a strain gauge configured to detect a compressive clamping load, for instance, being transmitted through the movable support 20590. The sensing system 20595 further comprises a longitudinally-extending conductive trace 20599 positioned within the cartridge channel 20585. In at least one instance, the conductive trace 20599 is positioned on a sidewall of the longitudinal channel slot defined in the cartridge channel 20582 so that the electrical contact 20598 is electrically coupled with the conductive trace 20599 regardless of where the movable cartridge support 20590 is positioned along the staple firing stroke path. In at least one instance, the conductive trace 20599 comprises a supply trace and a return trace. In at least one embodiment, a supply trace is positioned on one side of the longitudinal channel slot and a return trace is positioned on the opposite side of the longitudinal channel slot. In at least one embodiment, one trace is positioned within the longitudinal channel slot and another trace is positioned on or within a cartridge deck surface of the cartridge body, for example.

The sensing system 20595 is coupled to a control circuit, for example, configured to monitor the load detected by the sensing system 20595. In various embodiments, the control circuit is configured to control the operation of a surgical stapling instrument—and use data from the sensing system 20595 to control the operation of the surgical stapling instrument, as described in greater detail below. In at least one embodiment, the control system is configured to control an electric motor, or closure motor, actuatable to clamp the end effector jaws of the surgical stapling instrument and/or an electric motor, or firing motor, actuatable to move a firing driver through a staple firing stroke. In at least one instance, the control circuit is configured to monitor the positon of the movable cartridge support 20590 in addition to monitoring the load detected by the sensing system 20595. The control circuit can also monitor a position of the sled and/or firing driver during the firing stroke. Such embodiments can allow a control circuit to determine a tissue pressure at a specific location within the firing stroke.

In various embodiments, further to the above, the control circuit is configured to utilize the position of the sled, the position of the firing driver, the position of the movable cartridge support 20590, and/or the load detected by the sensing system 20595 to control the staple firing stroke. In at least one embodiment, the control circuit is configured to operate the firing motor at a predetermined, or preset, speed when the staple firing stroke is actuated. Based on data from the sensing system 20595, however, the control circuit is configured to operate the firing motor at a slower speed and/or at a faster speed than the predetermined speed. For example, after the jaws of the end effector have been clamped onto patient tissue—but prior to performing the staple firing stroke—the detected clamping load may exceed a predetermined threshold load and, in response, the control circuit is configured to automatically adjust the predetermined speed of the firing motor prior to the staple firing stroke. In at least one instance, the excessive clamping load indicates that the tissue captured between the jaws of the end effector is thick and/or dense and, as a result, the control circuit operates the firing motor at a slower speed. In various instances, the control circuit adjusts the predetermined speed to a lower predetermined speed when the detected clamping load exceeds the threshold. In at least one embodiment, the control circuit adjusts the predetermined speed of the firing motor as a function of detected clamping load. In at least one such embodiment, the amount by which the control circuit adjusts speed of the firing motor is proportional to the amount in which the detected clamping load exceeds the threshold. In at least one embodiment, the control circuit comprises a processor and a pulse width modulation circuit and/or a frequency modulation circuit, for example, that can be used to operate the firing motor at a slower speed. In addition to or in lieu of adjusting the speed of the firing motor, the control circuit can be configured to adjust the torque that can be delivered by the firing motor. The control circuit is also configured to adjust the speed and/or torque of the firing motor during the staple firing stroke based on data received from the sensing system 20595 during the staple firing stroke. In addition to or in lieu of limiting the speed of the firing motor when the detected clamping load is excessive, i.e., exceeds a predetermined value, the control circuit is also configured to increase the current to the firing motor or the current available to the firing motor.

In various instances, further to the above, the control circuit is configured to dynamically adjust the speed and/or torque of the firing motor during different portions of the staple firing stroke. In at least one instance, a staple firing stroke has several portions—a first portion in which the movable cartridge support 20590 is stationary while a firing driver is moved toward the cartridge support 20590 by the firing motor, and a second portion in which the cartridge support 20590 is being moved distally by the firing driver. In at least one such instance, the control circuit utilizes a first set of parameters to operate the firing motor during the first portion of the staple firing stroke and a second set of parameters to operate the firing motor during the second portion of the staple firing stroke. In at least one instance, the control circuit is configured to evaluate the clamping load at the movable cartridge support 20590 prior to the staple firing stroke and/or when the staple firing stroke is initiated to establish the parameters used to operate the firing motor during the first portion of the staple firing stroke and then re-evaluate the clamping load at the movable cartridge support 20590 when the sled and/or firing driver are at and/or near the movable cartridge support 20590. At such point, new parameters are selected for the rest of the staple firing stroke. In some instances, though, the new parameters used during the second portion of the staple firing stroke are the same as the parameters used during the first portion of the staple firing stroke. In at least one instance, the control circuit is configured to automatically adjust motor parameters in anticipation of the additional resistance caused by having to push the movable cartridge support 20590 distally upon reaching the movable cartridge support 20590. These motor parameters can be selected based on not only the presence of the movable cartridge support 20590 but, also, based on the monitored clamping load at the movable cartridge support 20590.

In various embodiments, a staple cartridge comprises a plurality of pre-positioned cartridge supports, or pillars, where one or more of the supports are equipped with a sensing system such as the sensing systems disclosed herein. In at least one instance, a control circuit is configured to monitor a clamping load at each pre-positioned cartridge supports and is configured to adjust a motor control program based on the monitored loads at a plurality of locations.

In at least one instance, moving opposing jaws into a fully clamped position completes a sensing circuit within the end effector such that the sensing circuit is only powered upon attaining a fully clamped position with the jaws.

In at least one embodiment, the sensor 20597 is disconnected from the conductive trace 20599 upon being moved out of its initial position by the sled and/or firing driver, for example. In such an instance, the clamping load can be monitored by a control circuit via the conductive trace 20599 and the sensor 20597 while the movable cartridge support 20590 remains in its home position; however, once the cartridge support 20590 is moved distally out of its home position by the firing driver, the sensing circuit including the conductive trace 20599 and sensor 20597 is open and the control circuit is no longer in communication with the sensor 20597. The control circuit is configured to detect the open sensing circuit and take appropriate action. In at least one instance, for example, the control circuit is configured to utilize a different set of motor control parameters upon detecting that the sensor circuit is open. In at least one alternative embodiment, the sensor 20597 is substituted with a resistor and the sensor circuit is in a closed state while the cartridge support is in its home, or unadvanced, position. When the cartridge support is advanced distally from its home position, the sensing circuit remains closed through the remainder of the staple firing stroke but the resistance of the sensing circuit changes depending on the position of cartridge support. The sensing circuit and cartridge support can act as a potentiometer varying the resistance of the sensing circuit as the firing stroke progresses.

In at least one embodiment, a conductive trace is mounted to a movable cartridge support, such as the movable cartridge support 20590, for example. In at least one such embodiment, the conductive trace straddles the cartridge support. The conductive trace mounted to the movable cartridge support is configured to close a sensing circuit when the firing driver reaches the end of its staple firing stroke. The sensing circuit is in communication with a control circuit such that the control circuit is configured to determine that the firing driver, the sled, and/or the movable cartridge support 20590 has reached the end-of-stroke position. At such point, the control circuit is configured to stop and/or reverse the firing motor.

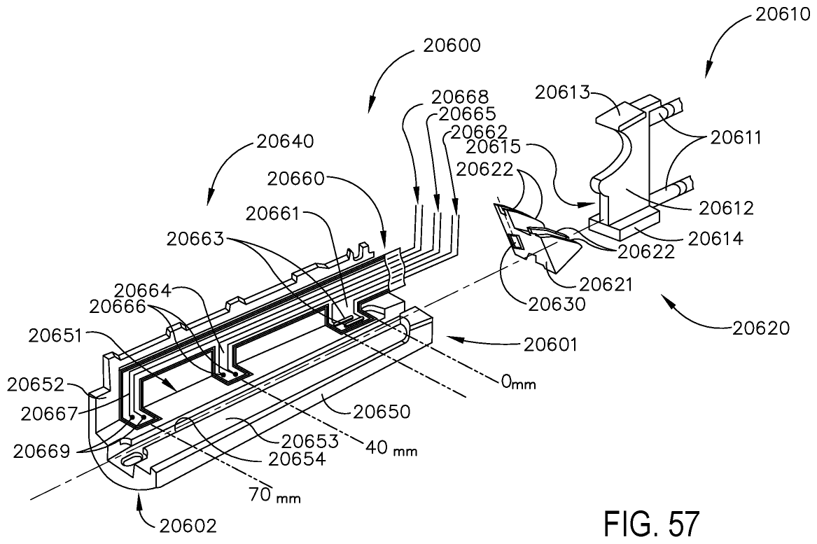

In at least one instance, an electrical circuit is utilized to determine the stroke position of a sled within an end effector. FIG. 57 depicts a surgical stapling assembly 20600 comprising a firing assembly 20610, a sled 20620 configured to be pushed through a firing stroke by the firing assembly 20610, and a cartridge channel jaw 20640. The sled 20620 is part of a replaceable staple cartridge seatable in the cartridge channel jaw 20640; however, other embodiments are envisioned in which the surgical stapling assembly 20600 comprises a loading unit that does not have readily replaceable components. The firing assembly 20610 comprises push rods 20611 and an I-beam head 20612 attached to distal ends of the push rods 20611. The push rods 20611 are translatable by any suitable means for moving the I-beam head 20612 longitudinally through the cartridge channel jaw 20640 such as, for example, by a motor. The I-beam head 20612 comprises a first pin 20613 configured to engage an anvil jaw of the surgical stapling assembly 20600 and a second pin 20614 configured to engage the cartridge channel jaw 20640. The pins 20613, 20614 are configured to travel within corresponding slots in the anvil jaw and cartridge channel jaw 20640 during a firing stroke to control the distance between a deck surface of a staple cartridge and a tissue compression surface of the anvil jaw. The I-beam head 20612 further comprises a distal end 20615 that is configured to abut and push the sled 20620 through the staple firing stroke. The sled 20620 is actuatable through the staple firing stroke by the I-beam head 20612 to eject staples from the staple cartridge. The sled 20620 comprises a base portion 20621 and ramped wedges 20622 extending from the base portion 20621. The ramped wedges 20622 are configured to engage staple drivers to eject staples from the staple cartridge during the firing stroke. The sled 20620 further comprises an electrically conductive trace 20630, discussed in greater detail below, that is in communication with a control circuit that can be used to track the position of the sled 20620.

Further to the above, the cartridge channel jaw 20640 comprises a cartridge channel 20650 configured to receive a replaceable staple cartridge in a channel cavity 20651 defined in the cartridge channel 20650. The channel cavity 20651 is defined by a bottom support surface 20653 and laterally-opposing channel walls 20652, one of which has been removed in FIG. 57 for to better illustrate various components positioned in the cartridge channel jaw 20640. The cartridge channel 20650 further comprises a longitudinal slot 20654 defined in the bottom support surface 20653 that is configured to receive at least a portion of the I-beam head 20612 therein during the firing stroke. The cartridge channel 20650 further comprises a longitudinal channel 20655 defined in a base portion 20656 of the cartridge channel 20650 that is configured to receive the second pin 20614 during the firing stroke.

The surgical stapling assembly 20600 further comprises a flex circuit 20660 positioned within the cartridge channel 20650. The flex circuit 20660 is attached to at least one of the channel walls 20652 of the cartridge channel 20650 via one or more adhesives, for example. That said, the flex circuit 20660 can be attached to the cartridge channel 20650 in any suitable manner. The flex circuit 20660 comprises a plurality of branches. For instance, the flex circuit 20660 comprises a proximal circuit branch 20661 positioned within a proximal zone of the firing stroke, an intermediate circuit branch 20664 positioned within an intermediate zone of the firing stroke, and a distal circuit branch 20667 positioned within a distal zone of the firing stroke. The circuit branches 20661, 20664, 20667 comprise flex circuit portions extending down onto the bottom support surface 20653 of the cartridge channel 20650.

The proximal circuit branch 20661 comprises electrical leads 20662 electrically connected to a control circuit of a surgical stapling system. Similarly, the intermediate circuit branch 20664 comprises electrical leads 20665 electrically connected to the control circuit and the distal circuit branch 20667 comprises electrical leads 20668 connected to the control circuit. The proximal circuit branch 20661 comprises electrical contacts 20663 electrically connected to the electrical leads 20662. Similarly, the intermediate circuit branch 20664 comprises electrical contacts 20666 electrically connected to the electrical leads 20665 and the distal circuit branch 20667 comprises electrical contacts 20669 electrically connected to the electrical leads 20668. The electrical contacts 20663, 20666, 20669 each comprise a discrete location for detecting the position of the sled 20620 as the sled 20620 is advanced distally during the staple firing stroke. When the sled 20620 is in its proximal-most unfired position, the trace 20630 on the sled 20620 electrically connects the contacts 20663 such that the proximal circuit branch 20661 is in a closed state indicating to the control circuit that the sled 20620 is in its proximal-most unfired position. When the sled 20620 is part of a replaceable staple cartridge, the closed state of the proximal circuit branch 20661 can also indicate to the control circuit that the replaceable staple cartridge is in an unfired state. Similarly, when the sled 20620 is in its distal-most fired position, the distal circuit branch 20667 is in a closed state indicating that the sled 20620 is in its distal-most fired position and that the staple cartridge has been fully fired. In at least one instance, the control circuit is configured to automatically operate the firing motor in reverse to retract the I-beam head 20612 by way of a motor upon detecting the closed state of the distal circuit branch 20667.

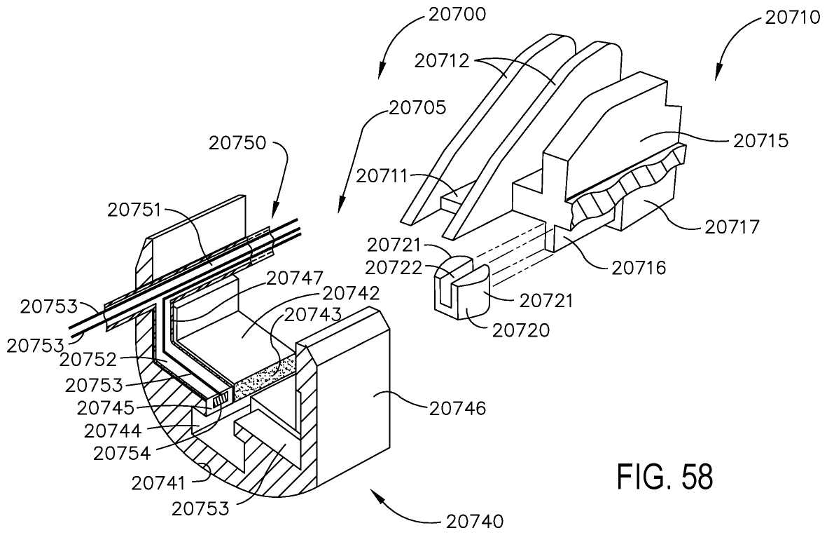

FIGS. 58-62 depict a surgical stapling assembly 20700 comprising a cartridge channel 20740 configured to receive a replaceable staple cartridge including a cartridge body and a sled 20710, and a sled detection system 20705 configured to detect the location of the sled 20710 within the cartridge channel 20740 during a staple firing stroke. The sled 20710 comprises a base portion 20711, ramped wedges 20712 extending from the base portion 20711 that are configured to eject staples from the staple cartridge body during the firing stroke, and a central portion 20715 configured to travel within a longitudinal slot defined in the cartridge body. The central portion 20715 comprises a lower distal rib 20716 comprising a first width, and a lower proximal rib 20717 comprising a second width which is greater than the first width. The cartridge channel 20740 comprises a bottom 20741 and channel sidewalls 20746 extending from the bottom 20741 that receive the replaceable staple cartridge. The bottom 20741 comprises a supporting surface 20742 configured to support the sled 20710 during the firing stroke. The bottom 20741 further comprises a longitudinal slot 20743 and a longitudinal channel 20744 defined therein. The longitudinal slot 20743 is configured to receive at least a portion of the lower distal rib 20716 and lower proximal rib 20717 of the sled 20710. The longitudinal slot 20743 is further configured to receive at least a portion of a firing driver during the firing stroke. In at least one such instance, the longitudinal channel 20744 is configured to receive a cam, or pin, of an I-beam head, for example, during the firing stroke.

The sled detection system 20705 is configured to detect the position of the sled 20710 within the cartridge channel 20740 during the firing stroke. In at least one instance, the sled detection system 20705 is electrically coupled to a control circuit of a surgical stapling instrument. The sled detection system 20705 comprises a conductive post 20720 mounted to the sled 20710 that is positioned within the longitudinal slot 20743 and a flex circuit 20750 positioned within the cartridge channel 20740 configured to be engaged by the conductive post 20720 during the firing stroke. In at least one embodiment, the sled 20710 and the conductive post 20720 are not connected to one another at the outset of the staple firing stroke. In such embodiments, the sled 20710 engages the conductive post 20720 at the outset of the staple firing stroke. The conductive post 20720 comprises a slot 20722 and conductive side walls 20721 and, during the initial distal motion of the sled 20710 during the staple firing stroke, the lower distal rib 20716 is configured to slide into the slot 20722 of the conductive post 20720. At such point, the lower proximal rib 20717 is configured to push the conductive post 20720 distally through the longitudinal slot 20743 during the firing stroke. In other embodiments, the sled 20710 the conductive post 20720 are connected to one another before the staple firing stroke.

The flex circuit 20750 is positioned within a circuit channel 20747 defined in the cartridge channel 20740. The circuit channel 20747 comprises a channel or slot sized and dimensioned such that the flex circuit 20750 is recessed with respect to, or flush with respect to, the inner surfaces of the cartridge channel 20740. As such, the flex circuit 20750 is not likely to interfere with installation of the replaceable staple cartridge into the cartridge channel 20740. The flex circuit 20750 comprises a plurality of branches 20752—each of which includes an electrical wire, or lead, 20753 and an electrical contact 20754. The branches 20752 of the flex circuit 20750 are arranged in pairs such that the contact 20754 of one branch 20752 is positioned on one side of the longitudinal slot 20743 and the contact 20754 of the other branch 20752 is positioned on the opposite side of the longitudinal slot 20743. As a result, each pair of branches 20752 comprises opposing electrical contacts 20754 that are contacted by the conductive post 20720 as the conductive post 20720 passes the electrical contacts 20754 during the staple firing stroke. As the conductive post 20720 passes a pair of opposing electrical contacts 20754, the conductive post 20720 closes an electrical circuit of the opposing electrical contacts 20754 and electrical leads 20753. As the conductive post 20720 moves past the pair of closed opposing contacts 20754, the electrical circuit opens. Progressive closing and opening of the electrical circuits of each pair of opposing secondary circuit branches 20752 during the firing stroke can indicate the position of the conductive post 20720. The position of the conductive post 20720 can be indicative of the position of the sled 20710 and/or the firing driver pushing the sled 20710 distally.

In various embodiments, further to the above, the conductive post 20720 is slideably positioned within the cartridge channel 20740 and does not comprise part of the replaceable staple cartridge. FIG. 59 depicts the sled 20710 positioned in an unfired position prior to the sled 20710 engaging the conductive post 20720 in a first zone 20707. The conductive post 20720 is pre-positioned in the longitudinal slot 20743 in the first zone 20707 where the conductive post 20720 closes the circuit of the opposing branches 20752 in the first zone 20707 prior to the beginning of the staple firing stroke. As can be seen in FIG. 60, the conductive post 20720 is engaged by the sled 20710 after an initial portion of the firing stroke. From this position, the sled 20710 is advanced further distally during the firing stroke where the conductive post 27620 is pushed out of engagement with the opposing electrical contacts 20754 in the first zone 20707 thereby opening the circuit of the branches 20752 in the first zone 20707. The sled 20710 is then pushed further distally through the firing stroke toward the branches 20752 in a second zone 20708 (FIG. 61) where the conductive post 20720 closes the circuit of the opposing branches 20752 in the second zone 20708. The sled 20710 is advanced further distally during the staple firing stroke where the conductive post 27620 is pushed out of engagement with the opposing electrical contacts 20754 in the second zone 20708 thereby opening the circuit of the branches 20752 in the second zone 20708. The sled 20710 is then pushed further distally through the staple firing stroke toward the branches 20752 in a third zone 20709 where the conductive post 20720 closes the circuit of the opposing branches 20752 in the third zone 20709 (FIG. 61) completing the staple firing stroke.

After the staple firing stroke has been completed, further to the above, the sled 20710 is retracted proximally. Referring to FIG. 62, the conductive post 20720 remains in the third zone 20709 as the sled 20710 is retracted back to an unfired position. In such instances, the conductive post 20720 releases from the lower distal rib 20716 as the sled 20710 is retracted. In other embodiments, the conductive post 20720 is not released from the sled 20710 and is retracted proximally with the sled 20710 into an unfired position. In at least one instance, the conductive post 20720 disconnects from the sled 20710, reseats, or is dropped off, at the first zone 20707 (into the position shown in FIG. 59) to prepare for another firing stroke of an unfired staple cartridge.

In various embodiments, a replaceable staple cartridge comprises a cartridge circuit including a memory device configured to store at least one datum related to whether or not the staple cartridge had been previously fired. The cartridge circuit is configured to write data onto the memory device which is accessible by the control circuit of a stapling instrument, for example, to determine the spent status of the staple cartridge once the staple cartridge has been installed in the stapling instrument. In instances where the control circuit receives data from the installed staple cartridge indicating that the staple cartridge has been previously fired, the control circuit prevents the firing motor from being operated to perform a staple firing stroke. If the control circuit does not receive data from the installed staple cartridge indicating that the staple cartridge has been previously fired, the control circuit permits the firing motor to respond to an input to perform a staple firing stroke.

In at least one instance, the conductive post 20720 provides support within the cartridge channel 20740 so as to help reduce deflection of the cartridge channel 20740 under clamping loads during the firing stroke. The conductive post 20720 can act as a wedge so as to help prevent the longitudinal slot 20743 from collapsing in on itself when subjected to a compressive load when the end effector is clamped onto patient tissue and/or during the staple firing stroke.

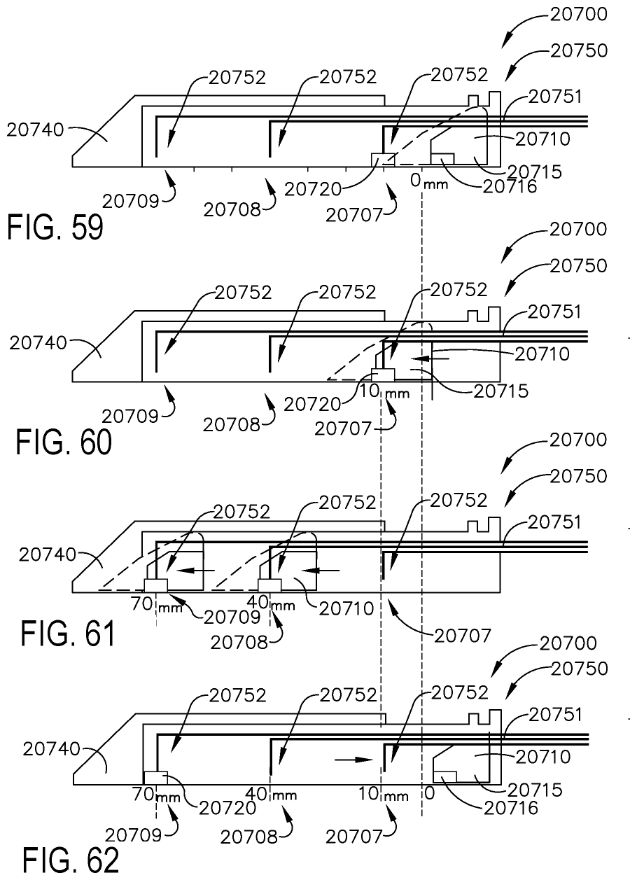

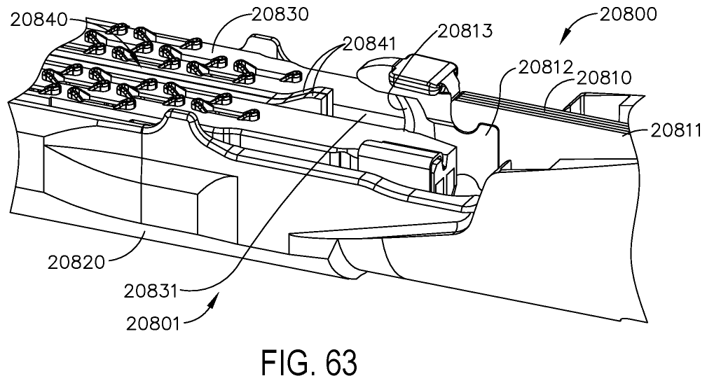

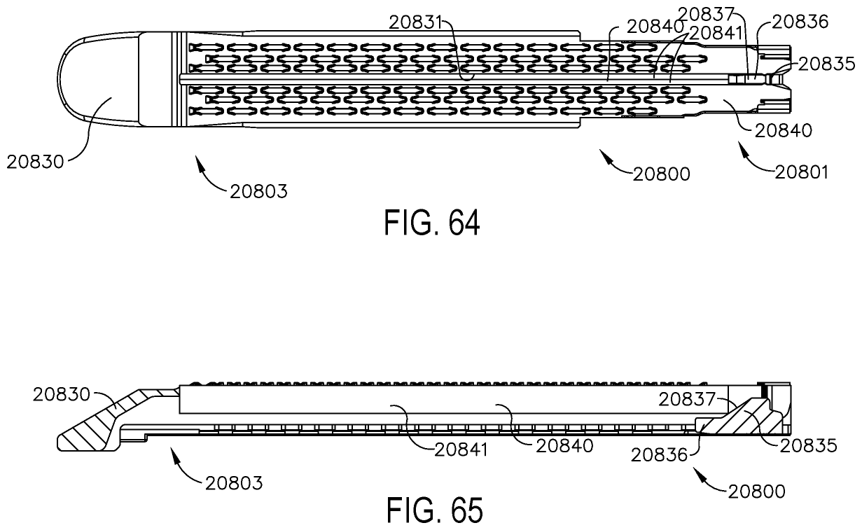

FIGS. 63-65 depict a surgical stapling assembly 20800 comprising a firing driver, or member, 20810, a cartridge channel 20820, and a staple cartridge assembly 20830 comprising a deployable support 20840 configured to help reduce deflection of the staple cartridge assembly 20830 during a firing stroke. The firing driver 20810 comprises a firing bar 20811 actuatable by a motor, for example, and a distal I-beam head 20812 attached to the firing bar 20811. The distal I-beam head 20812 comprises a knife edge 20813 configured to cut patient tissue during the firing stroke. The distal I-beam head 20812 is configured to push a sled 20835 of the staple cartridge assembly 20830 during the firing stroke to eject staples from the staple cartridge assembly 20830 and control a tissue gap distance between the staple cartridge assembly 20830 and an anvil. The distal I-beam head 20812 comprises a first cam configured to engage the cartridge channel 20820 and a second cam configured to engage an anvil jaw of the surgical stapling assembly 20800 during the staple firing stroke.

The staple cartridge assembly 20830 comprises a longitudinal slot 20831 defined therein that is configured to receive at least a portion of the distal I-beam head 20812 during the staple firing stroke. In at least one instance, the deployable support 20840 is press-fit into the longitudinal slot 20831 and is configured to be deployed from the staple cartridge assembly 20830 during the staple firing stroke along a cut-line, for example. The sled 20835 comprises a central portion 20836 configured to be pushed by the distal I-beam head 20812 through the firing stroke. The central portion 20836 comprises a ramp surface 20837 configured to lift the deployable support 20840 out of the longitudinal slot 20831 during the staple firing stroke as the sled 20835 is pushed from a proximal end 20801 of the staple cartridge assembly 20830 to a distal end 20803 of the staple cartridge assembly 20830. The ramp surface 20837 is configured to engage and lift the deployable support 20840 out of, or at least partially out of, the longitudinal slot 20831. In at least one instance, the distal I-beam head 20812 comprises a central ramp surface configured to deploy the deployable support 20840.

The deployable support 20840 fits tightly within the longitudinal slot 20831 such that the deployable support 20840 can reduce the deflection of the staple cartridge assembly 20830 under clamping loads by filling the longitudinal slot 20831. The deployable support 20840 comprises multiple strips 20841 of material; however, the deployable support 20840 can comprise any suitable configuration. In at least one instance, for example, the deployable support 20840 comprises a single strip of material configured to be cut and deployed from the longitudinal slot 20831 during the staple firing stroke. In at least one instance, the deployable support 20840 comprises a rigidity sufficient to prevent, or at least inhibit, the staple cartridge from collapsing, as discussed herein. In at least one instance, the deployable support 20840 comprises a hemostatic agent. In at least one instance, the deployable support 20840 comprises one or more layers of an absorbable hemostat such as, for example, SURGICEL®. In at least one instance, the deployable support 20840 is comprised of oxidized regenerated cellulose, for example.

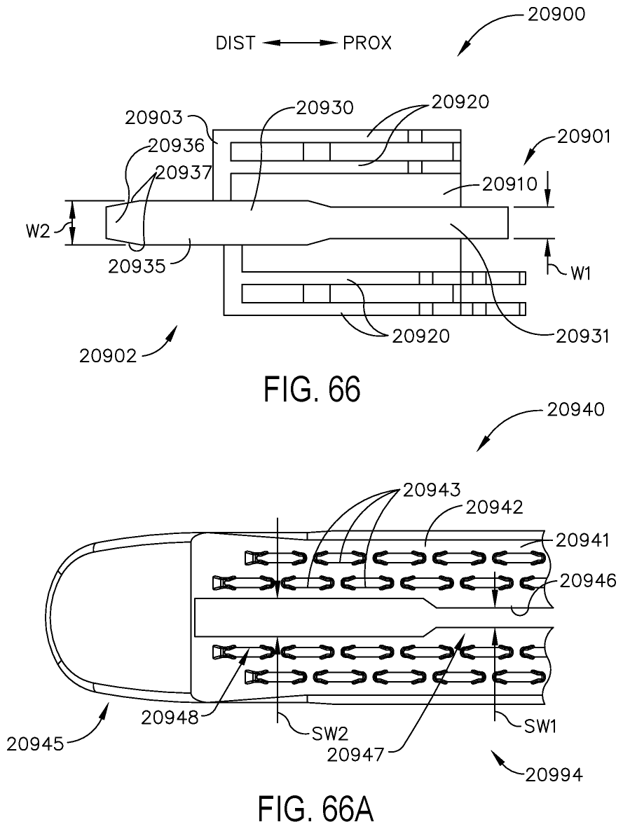

In various embodiments, as described above, a sled of a staple cartridge assembly comprises a central portion that moves within a central longitudinal slot of the staple cartridge assembly during a staple firing stroke that is configured to receive a tissue cutting knife, or a firing driver having a tissue cutting edge, during the staple firing stroke. FIG. 66 depicts a sled 20900 including a central portion configured to engage the sidewalls of the central longitudinal slot to prevent, or at least inhibit, a staple cartridge assembly from deflecting. The sled 20900 comprises a base portion 20910, ramped wedges 20920 extending from the base portion 20910 configured to eject staples from the staple cartridge assembly, and a central portion 20930 configured to be received within the central longitudinal slot. The sled 20900 comprises a proximal end 20901 and a distal end 20902. The central portion 20930 comprises a proximal portion 20931 and a distal portion 20935—each of which are configured to move through the central longitudinal slot. The proximal portion 20931 comprises a first width W1 and the distal portion 20935 comprises a second width W2 which is greater than the first width W1. The distal portion 20935 comprises a lead-in nose with wedge surfaces 20936 configured to help pry open a longitudinal slot of a staple cartridge assembly in the event that the staple cartridge assembly is collapsed, or deflected inwardly, for example, ahead of the sled 20900 as the sled 20900 moves distally. With the second width W2, the distal portion 20935 can provide support to the staple cartridge assembly by consuming the entire width of the central longitudinal slot during the staple firing stroke. In at least one instance, the second width W2 is greater than the width of the central longitudinal slot. In such an instance, the distal portion 20935 is configured to spread the central longitudinal slot during the firing stroke as the distal portion 20935 is advanced through the staple cartridge assembly. In at least one instance, the second width W2 is substantially equal to width of the central longitudinal slot so as to provide sufficient support within the central longitudinal slot but not unnecessarily increase friction between the sled 20900 and the central longitudinal slot during the firing stroke.

Further to the above, the distal portion 20935 of the sled 20930 is configured to support the staple cartridge assembly ahead of, i.e., distally with respect to, the staples being deployed during the staple firing stroke. The distal portion 20935 extends distally with respect to a front edge 20903 of the base portion 20910 and distally with respect to ramped wedges 20920 such that the distal portion 20935 can engage the sidewalls of the central longitudinal slot ahead of the staples being deployed. In at least one instance, the proximal portion 20931 further comprises a proximal-most section having a width equal to W2. Such a configuration of the central portion 20930 can create a long length of lateral support within the central longitudinal slot while having a narrow portion positioned intermediate the proximal and distal wide portions of the central portion 20930. The intermediate narrow portion of the central portion 20930 is sufficiently narrow such that it is not in contact with the sidewalls of the central longitudinal slot during the staple firing stroke. Such an arrangement can minimize, or at least reduce, the frictional forces between the central portion 20930 and the sidewalls of the longitudinal slot.

In various embodiments, further to the above, each of the ramped wedges 20920 further comprises a distal end having a lateral wedge configuration similar to the central portion 20930. Having distally-presenting lateral wedges at the distal end of each ramped wedge 20920 can reduce, or even reverse, the lateral deflection of a cartridge body. In various instances, the longitudinal cavities defined in the cartridge body through which the ramped wedges 20920 travel during the staple firing stroke may permit lateral deflection within the cartridge body that can cause the walls of the cartridge body to laterally collapse toward each other. In such instances, the distally-presented lateral wedges of each ramped wedge 20920 can pry open collapsed and/or partially collapsed adjacent cartridge walls. Moreover, a collection of distally-presented lateral wedges of a sled nose, sled ramps, and/or a distal I-beam head, for example, can collectively aid in reducing, or even reversing, lateral cartridge deflection.

In various embodiments, the sled 20900 shown in FIG. 66 can be used with a staple cartridge 20940 shown in FIG. 66A comprising a cartridge body 20941. The cartridge body 20941 comprises a deck 20942, a plurality of staple cavities 20943 defined in the deck, and a longitudinal slot 20946 defined in the cartridge body 20941. The longitudinal slot 20946 is configured to receive at least a portion of a firing driver and the central portion 20930 of the sled 20900. The sled 20900 is actuatable distally through the staple cartridge 20940 from a proximal end 20944 to a distal end 20945. The longitudinal slot 20946 comprises a proximal high-load zone 20947 having a first lateral slot width SW1 between opposing slot walls and a distal low-load zone 20948 having a second lateral slot width SW2 between opposing slot walls.

The central portion 20930 is configured to translate through the longitudinal slot 20946 during a firing stroke. In at least one instance, the slot widths SW1, SW2 are sized so as to provide maximum lateral support within the proximal high-load zone 20447 and reduce frictional forces, or force-to-fire, within the distal low-load zone 20448. In other words, the slot width SW1 is equal to or less than the width W2 of the central portion 20930 such that the distal portion 20935 supports and/or pries open the longitudinal slot 20946 within the proximal high-load zone 20447 providing lateral support to the cartridge body 20941 and reducing deflection of the cartridge body 20941. The slot width SW2 is equal to or greater than the width W2 so as to reduce friction between the central portion 20930 and the longitudinal slot 20946 and, thus, the force required to push the sled 20900 through the longitudinal slot 20946.

Further to the above, the wider slot width SW2 of the longitudinal slot 20496 is positioned distally with respect to the narrower slot width SW1. As a result of this arrangement, the force-to-fire the staple cartridge 20940 drops at the end of the staple firing stroke. In various other embodiments, however, the longitudinal slot 20496 can have any suitable arrangement of wider slot widths SW2 and narrower slot widths SW1.

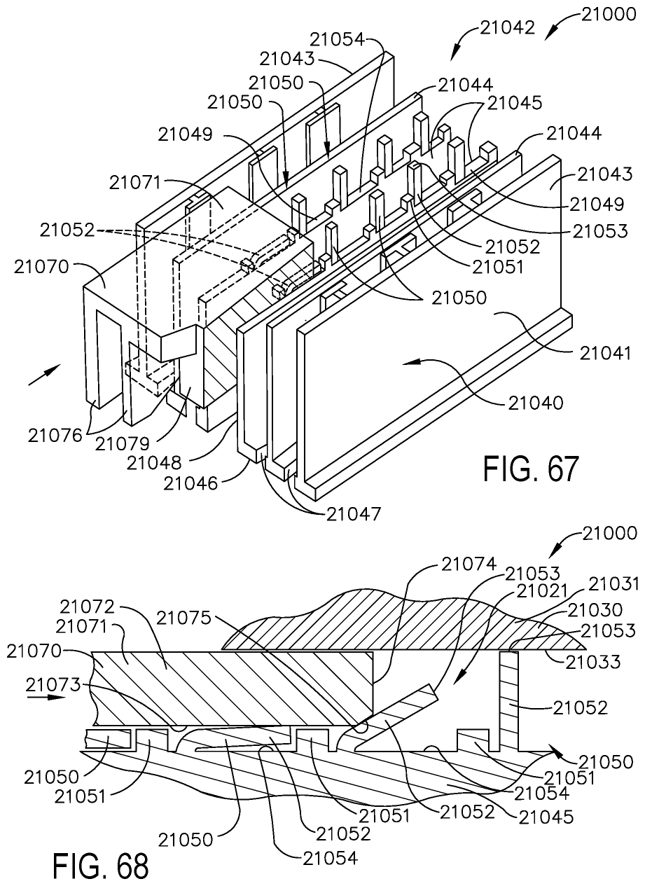

FIGS. 67-70 depict a surgical stapling assembly 21000 including features configured to reduce the deflection, or collapsing, of a staple cartridge under load. The surgical stapling assembly 21000 comprises an anvil jaw 21010, a cartridge channel jaw 21020, and a replaceable staple cartridge 21040 positionable in the cartridge channel jaw 21020. During a surgical procedure, tissue “T” can be clamped between the anvil jaw 21010 and the staple cartridge 21040. The anvil jaw 21010 comprises an anvil 21011 comprising a tissue-compression surface 21012, a plurality of staple forming pockets 21013 defined in the tissue-compression surface 21012, and a longitudinal slot 21014 configured to receive at least a portion of a firing driver therethrough during a staple firing stroke. In at least one instance, an anvil-camming pin of a firing driver is configured to be received within the longitudinal slot 21014 and is configured to positively position the anvil jaw 21010 relative to the cartridge channel jaw 21020 during the staple firing stroke.

The cartridge channel jaw 21020 comprises a cartridge channel 21030 configured to receive the staple cartridge 21040 therein. The cartridge channel 21030 comprises a bottom portion 21031 and channel sidewalls 21035 extending from the bottom portion 21031 to define a channel cavity 21036 within which the staple cartridge 21040 can be installed. The bottom portion 21031 comprises a longitudinal slot 21032 configured to receive at least a portion of the firing driver therethrough during the firing stroke. In at least one instance, a channel-camming pin of the firing driver is configured to be received within the longitudinal slot 21032 and is configured to positively position the cartridge channel jaw 21020 relative to the anvil jaw 21010 during the staple firing stroke. The bottom portion 21031 further comprises a bottom support surface 21033 opposite the cartridge body 21041, discussed in greater detail below.

The staple cartridge assembly 21040 comprises a cartridge body 21041, a sled 21070 movable through the cartridge body 21041 during the staple firing stroke by the firing driver, a plurality of staple drivers 21090 configured to be lifted by the sled 21070 during the firing stroke, and a plurality of staples 21095 supported by the staple drivers 21090. The cartridge body 21041 comprises a tissue-contacting deck surface 21046 having a plurality of staple cavities 21047 defined therein and a longitudinal slot 21048 configured to receive at least a portion of the firing driver during the firing stroke. The sled 21070 comprises a bottom web portion 21071 and a plurality of ramped wedges 21076 extending upwardly from the bottom web portion 21071 configured to sequentially lift the staple drivers 21090 from an unfired position (FIG. 69) to a fired position (FIG. 70) during the firing stroke to eject the staples 21095 from the cartridge body 21041 toward the anvil jaw 21010. The sled 21070 further comprises a central rib 21079 extending from a central web portion 21072 of the bottom web portion 21071 received within the longitudinal slot 21048. In at least one instance, the firing driver is configured to push the central rib 21079 to advance the sled 21070 distally during the staple firing stroke.

The sled 21070 is configured to be pushed distally from a proximal unfired position by the firing driver to eject the staples 21095. In order for the bottom web portion 21071 of the sled 21070 to translate through the cartridge channel jaw 21020 during the firing stroke, the cartridge channel jaw 21020 comprises a cavity, or space, 21021 defined between the cartridge body 21041 and the bottom support surface 21033 of the bottom portion 21031 of the cartridge channel 21030. The bottom web portion 21071 is configured to translate through the cavity 21021 during the firing stroke. Discussed in greater detail below, the staple cartridge assembly 21040 further comprises support features 21050 positioned within the cavity 21021 which are configured to support the cartridge body 21041 before the staple firing stroke. Discussed in greater detail below, the support features 21050 are configured to be pushed out of the way by the sled 21070 as the sled 21070 passes the support features 21050.

The cartridge body 21041 further comprises a plurality of cartridge walls 21042 extending vertically from the deck surface 21046. The plurality of cartridge walls 21042 define various cavities and spaces within the cartridge body 21041 itself such as, for example, the staple cavities 21047—the vertical spaces within which the staple drivers 21090 are lifted—and the longitudinal spaces within which the ramped wedges 21076 travel. The plurality of cartridge walls 21042 comprise outer walls 21043, intermediate walls 21044, and inner walls 21045. The outer walls 21043 are positioned adjacent to and supported by channel sidewalls 21035, the inner walls 21045 are positioned adjacent to and define the longitudinal slot 21048, and the intermediate walls 21044 are positioned between the outer walls 21043 and the inner walls 21045. As discussed above, the staple cartridge assembly 21040 comprises support features 21050. Specifically, the support features 21050 are positioned on a bottom surface 21049 of the inner walls 21045. The support features 21050 are configured to traverse the cavity 21021, or extend down toward the bottom support surface 21033 of the cartridge channel 21030, to provide vertical support to the inner walls 21045. The support features 21050 can help reduce inward and/or downward deflection of the inner walls 21045 of the cartridge body 21041 by extending down to the bottom support surface 21033. The bottom support surface 21033 can, as a result, counter vertical deflection of the inner walls 21045 by way of the support features 21050.

The support features 21050 comprise proximal support nubs 21051 and support pillars 21052 positioned adjacent and distal to the proximal support nubs 21051. In a fully-extended configuration, or non-hinged configuration, a bottom surface 21053 of the support pillar 21052 is vertically supported by the opposing bottom support surface 21033 of the cartridge channel 21030. Prior to the staple firing stroke, the support pillars 21052 are in the non-hinged configuration so as to provide support to the inner walls 20145. When the sled 21070 is moved through its staple firing stroke, the sled 21070 then hinges the support pillars 21052 out of the way upwardly into pillar cavities 21054. Referring to FIG. 68, the central web portion 21072 of the bottom web portion 21071 of the sled 21070 comprises a distal edge 21074 having a chamfered surface 21075. During the firing stroke, the distal edge 21074 is configured to engage and pivot, or hinge, the support pillars 21052 upwardly toward the pillar cavities 21054. The chamfered surface 21075 is configured to further encourage the rotation of the support pillars 21052 toward the pillar cavities 21054. Once the support pillars 21052 are fully collapsed into the pillar cavities 21054, the support pillars 21052 are held in the pillar cavities 21054 by a top surface 21073 of the central web portion 21072 as the sled 21070 passes thereby. Once the sled 21070 passes the support pillars 21052, the support pillars 21052 are configured to elastically reassume their fully-extended configuration to support the inner walls 21045 proximal of the firing stroke. In other embodiments, the support pillars 21052 do not reassume their fully-extended configuration and, instead, are plastically deformed after being engaged by the sled 21070. In any event, the support pillars 21052 can provide temporary or transient support to the cartridge body 21041.

As discussed above, the support features 21050 extend from the inner walls 21045 of the cartridge body 21041. In addition to or in lieu of the above, various embodiments are envisioned in which the support features 21050 are present on the intermediate walls 21044 and/or the outer walls 21043 of the cartridge body 21041. In various embodiments, the support pillars 21052 each have the same configuration; however, in other embodiments, at least some of the support pillars 21052 have different configurations. In at least one such embodiment, the support pillars 21052 are more robust, such as being thicker, for example, at the proximal end of the cartridge body 21041 as compared to the distal end of the cartridge body 21041. In at least one such embodiment, the support pillars 21052 are taller at the distal end of the cartridge body 21041 as compared to the proximal end of the cartridge body 21041. In various embodiments, the top surface 21073 of the central web portion 21072 of the sled 21070 comprises longitudinal slots defined therein within which the support pillars 21052 can slide as the sled 21070 passes thereby. Such an arrangement can reduce binding between the support pillars 21052 and the sled 21070. In various embodiments, the sled 21070 is configured to engage and break the support pillars 21052 as the sled 21070 passes each support pillar 21052. In at least one instance, some of the support pillars 21052 of the cartridge body 21041 are frangible and are configured to break when engaged by the sled while other support pillars 21052 are not frangible and are configured to elastically bend out of the way until the sled 21070 passes by the elastically deflected support pillars 21052.

In many instances, the staple cartridge of a surgical stapling end effector is particularly vulnerable, or susceptible, to bending-elastically and/or plastically—when subjected to a compressive load. This vulnerability can be attributed, at least in part, to the materials comprising the staple cartridge as compared to the materials comprising a cartridge channel jaw—within which a staple cartridge is positioned—and/or a jaw positioned opposite the staple cartridge such as, for example, an anvil jaw. In various instances, the staple cartridge is comprised of a plastic material while the cartridge channel jaw and the anvil jaw are comprised of a metal material, such as stainless steel, for example, and, because of the different elasticity, flexibility, and strength characteristics of such materials, the staple cartridge may tend to elastically deflect, and/or twist, from its original shape under certain loads, such as compressive loads, for example.

This vulnerability of the staple cartridge can also be attributed to the nature of how the components of surgical stapling end effectors fit together. More specifically, some surgical stapling end effectors can include a longitudinally translatable cutting member that traverses a longitudinal slot defined in the staple cartridge to cut tissue clamped between the jaws. The longitudinal slot can increase how much the staple cartridge and/or portions of the staple cartridge can twist or deflect from its original shape under load. Some surgical stapling end effectors also include a translatable sled comprising ramped wedges configured to eject staples from the staple cartridge during the staple firing stroke. In at least one instance, the sled comprises a base which requires space to translate through the surgical stapling end effector. This space, often times positioned between a cartridge body and a pan attached to the cartridge body, defines a void or cavity into which portions of the staple cartridge can deflect. In embodiments without a pan attached to the cartridge body, a similar void or cavity is present between the cartridge body and the cartridge channel jaw.

In various instances, staple cartridges can be prone to collapsing when subjected to loads. A staple cartridge collapses when the two opposing sides of the longitudinal slot extending within the staple cartridge rotate or bend inwardly. Forces capable of collapsing a staple cartridge can occur when patient tissue is clamped against the staple cartridge and/or when staples are fired from the staple cartridge during a staple firing stroke. In at least one instance, initial clamping forces can be applied to the staple cartridge when the jaws are approximated to clamp tissue therebetween or, in other words, during a clamping stroke. In at least one instance, a firing driver, such as an I-beam, for example, engages the cartridge channel jaw and the anvil jaw of a surgical stapling assembly during the firing stroke which creates a compressive load against the deck of the staple cartridge. In at least one instance, the location of the I-beam represents the location at which the staple cartridge may be most prone to collapsing.

The deflection of a staple cartridge can cause the staples being ejected from the staple cartridge to be misaligned with their corresponding anvil forming pockets. Such deflection may also cause tissue to bunch up near the longitudinal slot which can increase the difficultly of cutting the tissue during the staple firing stroke. Such deflection can also cause binding between various components of the surgical stapling assembly such as, for example, the staple drivers and staple cavities of the staple cartridge, the I-beam and the longitudinal slot of the staple cartridge, and/or the sled and the cartridge body of the staple cartridge, among others. Binding between such components can cause the components to jam and/or increase the required force to staple and cut tissue, for example.

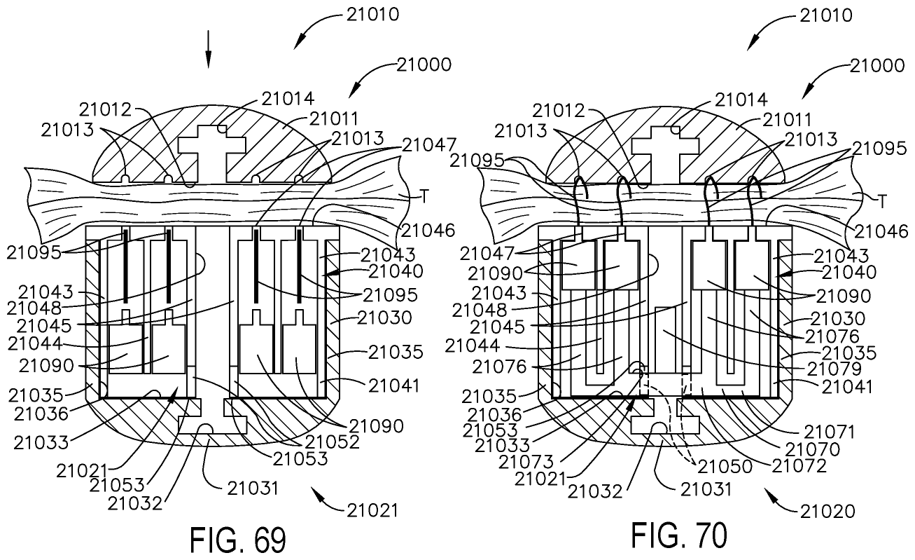

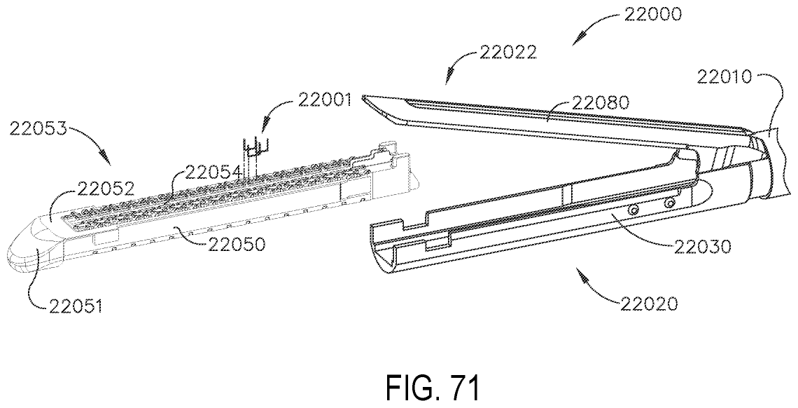

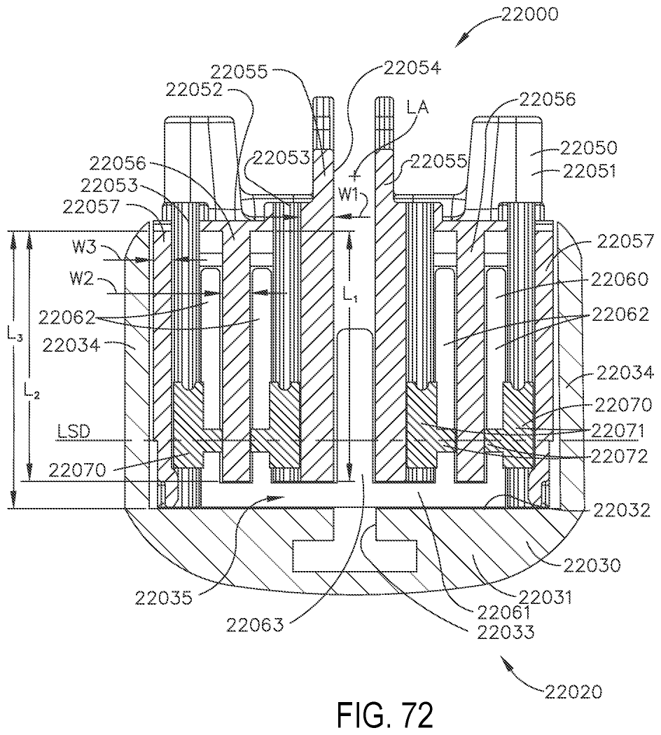

FIGS. 71 and 72 depict a surgical stapling assembly 22000 that is configured to clamp, staple, and cut patient tissue. The surgical stapling assembly 22000 comprises a shaft 22010, a cartridge channel jaw 22020, and an anvil jaw 22022 movable relative to the cartridge channel jaw 22020 to clamp tissue between the anvil jaw 22022 and a staple cartridge assembly 22050 positioned in the cartridge channel jaw 22020. The staple cartridge assembly 22050 is replaceable and is configured to be readily installed in, and removed from, the cartridge channel jaw 22020 during a surgical procedure; however, embodiments are envisioned in which a cartridge channel jaw comprises a staple cartridge assembly therein that is not replaceable or configured to be readily installed and removed from the cartridge channel jaw. Upon installation of the staple cartridge assembly 22050 into the cartridge channel jaw 22020, the surgical stapling assembly 22000 can be used to clamp, cut, and staple patient tissue. Details of various surgical stapling assemblies, components, and systems can be seen in U.S. Patent Application Publication No. 2021/0059672, which is hereby incorporated by reference in its entirety herein.

The cartridge channel jaw 22020 comprises a bottom 22031 and channel sidewalls 22034 extending from the bottom 22031. The bottom 22031 and the sidewalls 22034 define a cavity 22035 within which the staple cartridge assembly 22050 is configured to be positioned. The bottom 22031 comprises a cartridge-supporting surface 22032 and a longitudinal slot 22033 configured to receive at least a portion of a firing driver therein during a firing stroke. In various embodiments, the firing driver comprises a bar, a rod, a beam, an I-beam, and/or a tissue cutting knife, for example. The staple cartridge assembly 22050 comprises a cartridge body 22051 including a deck 22052 having staple cavities 22053 defined therein and a longitudinal slot 22054 defined in the deck 22052 configured to receive at least a portion of the firing driver during the firing stroke. The staple cartridge assembly 22050 further comprises a sled 22060 movable through the cartridge body 22051 by the firing driver during the firing stroke to engage staple drivers 22070 movably positioned in the staple cavities 22053 and drive staples 22001 stored in the staple cavities 22053 out of the cartridge body 22051.

Referring to FIG. 72, the sled 22060 comprises a bottom 22061 translatable longitudinally between the cartridge body 22051 and the cartridge channel 22030 and a plurality of ramped wedges, or sled ramps, 22062 extending upwardly from the bottom 22061. Further to the above, the ramped wedges 22062 are configured to sequentially lift the staple drivers 22070 from an unfired position (FIG. 72) to a fired position as the sled 22060 is moved through the cartridge body 22051 by the firing driver. In such instances, the staple drivers 22070 push the staples 22001 toward and against forming pockets defined in the anvil jaw 22022 to deform the staples 22001 into a formed configuration, such as a B-shaped configuration, for example.

Referring again to FIG. 72, each staple driver 22070 comprises a plurality of support columns 22071, each of which is configured to support a staple thereon. In at least one embodiment, each support column 22071 comprises a trough or cradle defined in a top surface thereof that is configured to receive a base of a staple 22001 therein. Each staple driver 22070 further comprises web portions 22072 extending laterally between the support columns 22071. The web portions 22072 are configured to be contacted by the ramped wedges 22062 of the sled 22060 during the firing stroke to lift the staple drivers 22070 relative to the cartridge body 22051. The staple cartridge assembly 22050 comprises six longitudinal rows of staple cavities where each side of the staple cartridge assembly 22050 comprises three longitudinal rows of staple cavities. More specifically, the cartridge body 22051 has two sides—one on each side of the longitudinal slot 22054—where each side has an inner row of staple cavities adjacent the longitudinal slot 22054, an outer row of staple cavities, and an intermediate row of staple cavities positioned intermediate the inner row and the outer row. The staple cavities in the inner row and the staple cavities in the outer row are aligned laterally with one another. The staple cavities in the intermediate row are not laterally aligned with the staple cavities in the inner row and the outer row. Rather, the staple cavities in the intermediate row are shifted longitudinally, or longitudinally offset, with respect to the staple cavities in the inner row and the outer row. Because the intermediate row of staple cavities is longitudinally offset, an intermediate support column is not seen in the cross-sectional view of FIG. 72.

Referring again to FIG. 72, the cartridge body 22051 further comprises a plurality of longitudinally-extending cartridge walls 22055, 22056, 22057 extending downwardly from the deck 22052. Defined between the cartridge walls 22055, 22056, 22057 are longitudinal slots, or cavities, within which the ramped wedges 22062 of the sled 22060 are configured to translate during the staple firing stroke. Cartridge walls 22055 comprise inner cartridge walls that define the longitudinal slot 22054, cartridge walls 22056 comprise intermediate cartridge walls, and cartridge walls 22057 comprise outer cartridge walls adjacent the channel sidewalls 22034. The intermediate cartridge walls 22056 are positioned laterally between the inner cartridge walls 22055 and the outer cartridge walls 22057.

The inner cartridge walls 22055 comprise an inner lateral width W1. The intermediate cartridge walls 22056 comprise a intermediate lateral width W2. The outer cartridge walls 22057 comprise an outer lateral width W3. The inner lateral width W1 is wider than the intermediate lateral width W2 and the intermediate lateral width W2 is wider than the outer lateral width W3. Such a variance in lateral widths between the cartridge walls 22055, 22056, 22057 defines a varying lateral width gradient so as to provide thicker cartridge walls closer to the longitudinal slot 22054 than the channel sidewalls 22034. In at least one instance, providing thicker cartridge walls closer to the longitudinal slot 22054 can help reduce cartridge deflection, twisting, and/or collapsing within the staple cartridge assembly 22050.

As discussed above, referring again to FIG. 72, the inner cartridge walls 22055 extend downwardly from the deck 22052 of the cartridge body 22051. As can be seen in FIG. 72, a portion of the sled 22060 extends under the inner cartridge walls 22055. When the deck 22052 is subjected to a compressive load, for example, the inner cartridge walls 22055 can be in contact with the sled 22060. In some instances, the inner cartridge walls 22055 and the sled 22060 are sized and configured such that the sled 22060 is not in contact with the inner cartridge walls 22055 until the cartridge body 22051 deflects under load. In other instances, the inner cartridge walls 22055 are in contact with the sled 22060 prior the cartridge body 22051 deflecting under load. Similarly, the intermediate cartridge walls 22056 can be in contact with the sled 22060 when the deck 22052 is subjected to a compressive load. In some instances, the intermediate cartridge walls 22056 and the sled 22060 are sized and configured such that the sled 22060 is not in contact with the intermediate cartridge walls 22056 until the cartridge body 22051 deflects under load. In other instances, the intermediate cartridge walls 22056 are in contact with the sled 22060 prior the cartridge body 22051 deflecting under load.

When the cartridge body 22051 deflects under load and the inner cartridge walls 22055 and the intermediate cartridge walls 22056 push downwardly on the sled 22060, as described above, the sled 22060 is in contact with and supported by the cartridge support surface 22032 of the channel jaw 22030. In such instances, the inner cartridge walls 22055 and the intermediate cartridge walls 22056 comprise pillars that stiffen the staple cartridge assembly 22050. Referring to the cross-section of the staple cartridge assembly 22050 depicted in FIG. 72, the inner cartridge walls 22055 have a pillar length L1 as measured downwardly from the bottom of the deck 22032. Similarly, the intermediate cartridge walls 22056 have a pillar length L2 as measured downwardly from the bottom of the deck 22032. The pillar length L1 of the inner cartridge walls 22055 is constant along the longitudinal length of the cartridge body 22050, i.e., between the proximal and distal ends of the cartridge body 22050; however, embodiments are envisioned where the pillar length L1 of the inner cartridge walls 22055 is different along the longitudinal length of the cartridge body 22050. Similarly, the pillar length L2 of the intermediate cartridge walls 22056 is constant along the longitudinal length of the cartridge body 22050; however, embodiments are envisioned where the pillar length L2 of the intermediate cartridge walls 22056 is different along the longitudinal length of the cartridge body 22050.

Moreover, further to the above, when a compressive load is applied to the deck 22052, the outer cartridge walls 22057 are in contact with and are supported by the cartridge support surface 22032 of the channel jaw 22030. In such instances, the outer cartridge walls 22057 comprise pillars that support the deck 22052. In the cross-section depicted in FIG. 72, the outer cartridge walls 22057 are defined by a pillar length L3. Similar to the above, the pillar length L3 is constant along the longitudinal length of the cartridge body 22050; however, embodiments are envisioned where the pillar length L3 of the outer cartridge walls 22057 is different along the longitudinal length of the cartridge body 22050. In other embodiments where a staple cartridge comprises a pan attached to and extending under the cartridge body, the sled 22060 is pushed downwardly against the pan and the outer cartridge walls 22057 are supported by the pan which are, in turn, supported by the cartridge support surface 22032.

Further to the above, the inner cartridge walls 22055, the intermediate cartridge walls 22056, the outer cartridge walls 22057, and the sled 22060 co-operate to support the cartridge body 22051. In various instances, the inner cartridge walls 22055, the intermediate cartridge walls 22056, the outer cartridge walls 22057, and the sled 22060 co-operate to support the cartridge body 22051 within a support plane that is transverse to the longitudinal axis of the staple cartridge assembly 22050. The cross-section of the staple cartridge assembly 22050 in FIG. 72 is taken along such a plane. Referring to FIG. 72, a longitudinal axis LA of the staple cartridge assembly 22050 is defined by the longitudinal slot 22054 which extends along the center of the cartridge body 22051 and the support plane of FIG. 72 is orthogonal to the longitudinal axis LA. In this support plane, the inner cartridge walls 22055 have a lateral width W1, the intermediate cartridge walls 22056 have a lateral width W2, and the outer cartridge walls 22057 have a lateral width W3. Notably, the lateral width W1 is constant along the entire pillar length L1 of the inner cartridge walls 22055 in this support plane. However, embodiments are envisioned in which the lateral width W1 of an inner cartridge wall 22055 is not constant along its entire pillar length L1. Similarly, the lateral width W2 is constant along the entire pillar length L2 of the intermediate cartridge walls 22056 and the lateral width W3 is constant along the entire pillar length L3 of the outer cartridge walls 22057 in this support plane. Whether or not the lateral width W1 of an inner cartridge wall 22055 is constant along the pillar length L1, an average lateral width of the inner cartridge wall 22055, i.e., the pillar width PW1, can be calculated along the pillar length L1. Similarly, an average lateral width of an intermediate cartridge wall 22056, i.e., the pillar width PW2, can be calculated along the pillar length L2 and an average lateral width of an outer cartridge wall 22057, i.e., the average pillar width PW3, can be calculated along the pillar length L3. In various embodiments, further to the above, the average pillar width PW1 of the inner cartridge walls 22055 is wider than the average pillar width PW2 of the intermediate cartridge walls 22056, and the average pillar width PW2 of the intermediate cartridge walls 22056 is wider than the outer cartridge walls 22057.

Further to the above, the support plane depicted in FIG. 72 extends through, or transects, the inner cartridge walls 22055, the intermediate cartridge walls 22056, the outer cartridge walls 22057, and the sled 22060. During a staple firing stroke, the sled 22060 moves from a proximal unfired position at the proximal end of the cartridge body 22051 to a distal fired position at the distal end of the cartridge body 22051. As a result, the region in which the sled 22060 supports the cartridge body 22051 moves from the proximal end of the cartridge body 22051 to the distal end of the cartridge body 22051 during the staple firing stroke. Moreover, as a result, a support plane orthogonal to the longitudinal axis LA can move from the proximal end of the cartridge body 22051 to the distal end of the cartridge body 22051. In at least one such instance, the support plane moves with the sled 22060 during the staple firing stroke from the proximal unfired position of the sled 22060 to the distal fired position of the sled 22060.

Further to the above, the lateral width W1 and/or the pillar width PW1 of the inner cartridge walls 22055 is constant along the longitudinal length thereof. As a result, the lateral width W1 and/or the pillar width PW1 of the inner cartridge walls 22055 is the same at the proximal end of the staple firing stroke as the distal end of the staple firing stroke. In other embodiments, the lateral width W1 and/or the pillar width PW1 of the inner cartridge walls 22055 is not constant along the longitudinal length thereof. In at least one such embodiment, the pillar width PW1 of the inner cartridge walls 22055 at the proximal end of the staple firing stroke is wider than the pillar width PW1 of the inner cartridge walls 22055 at the distal end of the staple firing stroke. Similarly, the lateral widths W2 and W3 and/or the pillar widths PW2 and PW3 of the cartridge walls 22056 and 22057, respectively, are constant along the longitudinal length thereof. As a result, the lateral widths W2 and W3 and/or the pillar widths PW2 and PW3 of the cartridge walls 22056 and 22057 are the same at the proximal end of the staple firing stroke as the distal end of the staple firing stroke. In other embodiments, the lateral widths W2 and W3 and/or the pillar widths PW2 and PW3 of the cartridge walls 22056 and 22057 are not constant along the longitudinal length thereof. In at least one such embodiment, the pillar width PW2 of the intermediate cartridge walls 22056 at the proximal end of the staple firing stroke is wider than the pillar width PW2 of the intermediate cartridge walls 22056 at the distal end of the staple firing stroke. Likewise, in at least one such embodiment, the pillar width PW3 of the outer cartridge walls 22057 at the proximal end of the staple firing stroke is wider than the pillar width PW3 of the outer cartridge walls 22057 at the distal end of the staple firing stroke.

Further to the above, referring again to FIG. 72, the cartridge body 22051 comprises two inner cartridge walls 22055 that have the same lateral width W1 and pillar width PW1 for any given support plane that is orthogonal to the longitudinal axis LA. Such an arrangement can permit the cartridge body 22051 to deflect symmetrically when subjected to a compressive load in various instances. In other embodiments, however, one of the inner cartridge walls 22055 can have a different lateral width W1 and/or pillar width PW1 than the other inner cartridge wall 22055. Similarly, the cartridge body 22051 comprises two intermediate cartridge walls 22056 that have the same lateral width W2 and pillar width PW2 and two outer cartridge walls 22057 that have the same lateral width W3 and pillar width PW3 for any given support plane that is orthogonal to the longitudinal axis LA. In other embodiments, one of the intermediate cartridge walls 22056 can have a different lateral width W2 and/or pillar width PW2 than the other intermediate cartridge wall 22056 and/or one of the outer cartridge walls 22057 can have a different lateral width W3 and/or pillar width PW3 than the other outer cartridge wall 22057.

Further to the above, the cartridge channel jaw 22020 defines a lateral support datum LSD along which a configuration of components of the cartridge channel jaw 22020 can aid in countering lateral cartridge deflection, twisting, and/or collapsing, for example, of the staple cartridge assembly 22050. Material, via the components of the staple cartridge assembly 22050, is presented in the lateral support datum LSD to consume laterally-presented spaces, or cavities, along the lateral support datum LSD thereby providing a path for laterally-induced force or stress to be transferred to the channel sidewalls 22034 of the channel jaw 22030. For instance, the cartridge body 22051 comprises staple cavities 22053 defined therein and staple drivers 22070, which move within the staple cavities 22053, that are sized and configured such that support columns 22071 of the staple drivers 22070 consume the empty spaces created by the staple cavities 22053. In such instances, as a result, the staple drivers 22070 comprise surfaces that are in abutting contact with the inner cartridge walls 22055, the intermediate cartridge walls 22056, and the outer cartridge walls 22057 such that the staple drivers 22070 provide lateral support to the cartridge body 22051. Moreover, the staple drivers 22070 comprise web portions 22052 that are slidingly engaged with the intermediate cartridge walls 22056 which provide lateral support to the cartridge body 22051. Also, the central portion 22063 of the sled 22060 is fitted in the longitudinal slot 22054 within the lateral support datum LSD so as to provide lateral support to the cartridge body 22051 within the longitudinal slot 22054.

Under a clamping load at the location within which the cross-sectional view of FIG. 72 is taken, for example, the cartridge walls may deflect inwardly into the longitudinal slot 22054 and/or outwardly toward the channel sidewalls 22034. Without material in each laterally-presented space between the longitudinal slot 22054 and the channel sidewalls 22034, the cartridge body 22051 may collapse inwardly on itself an/or outwardly toward the channel sidewalls 22034. With material in each laterally-presented space along the lateral support datum LSD by, for example, the central portion 22063 of the sled 22060, the inner cartridge walls 22055, the support columns 22071, the web portions 22072 of the staple drivers 22070, the intermediate cartridge walls 22056, and the outer cartridge walls 22057, the load is transferred through these components and to the channel sidewalls 22034. In at least one instance, the channel sidewalls 22034 are comprised of a stronger, or stiffer, material relative to the staple cartridge assembly and can absorb and/or effectively counter the clamping load.

The absorption of the clamping load by the channel sidewalls 22034 can occur throughout the firing stroke by utilizing one or more lateral support datum LSD. In at least one instance, the lateral support datum LSD is dynamically sustained with the central portion 22063 of the sled 22060 and with the staple drivers 22070 being lifted or driven by the sled 22060 such that the lateral support datum LSD not only moves longitudinally through the staple cartridge assembly 22050 during the firing stroke but also moves vertically as the staple drivers 22070 are lifted relative to the cartridge body 22051 and the staples 22001 are fired.

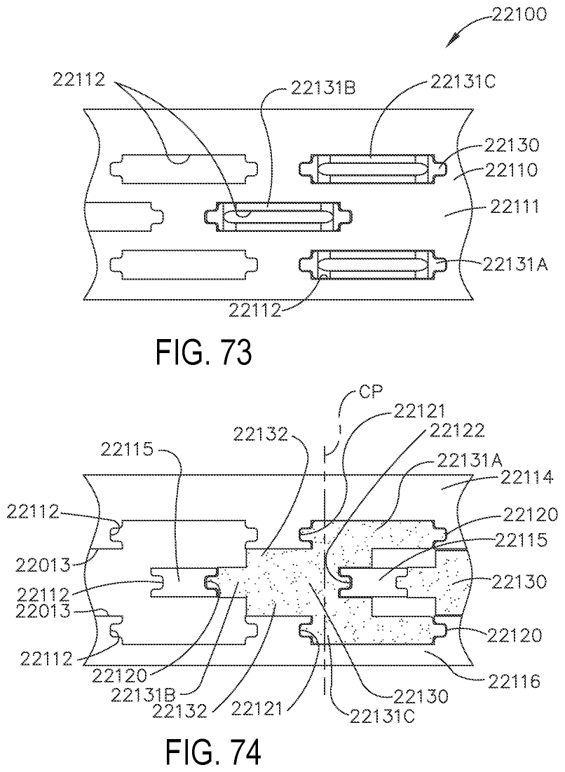

FIGS. 73 and 74 depict a staple cartridge assembly 22100 comprising a cartridge body 22110 and a plurality of staple drivers 22130 configured to be sequentially lifted relative to the cartridge body 22110 by a sled during a staple firing stroke. The cartridge body 22110 comprises a deck 22111 configured to support patient tissue and a plurality of staple cavities 22112 defined in the deck 22111. The cartridge body 22110 further comprises inner cartridge walls 22114, intermediate cartridge wall pillars 22115, and outer cartridge walls 22116. Each staple driver 22130 is configured to support and drive three staples; however, other embodiments are envisioned in which one or more staple drivers of the staple cartridge assembly 22100 are configured to support and drive more than three staples or less than three staples. Each staple driver 22130 comprises an inner support column 22131A, an intermediate support column 22131B, and an outer support column 22131C. The inner support column 22131A and the outer support column 22131C are proximal to the intermediate support column 22131B. The support columns 22131A, 22131B, 22131C are connected by driver bridges 22132. In at least one instance, the driver bridges 22132 are configured to be engaged by corresponding ramps defined on the sled during the staple firing stroke to lift the staple driver 22130, and the staples supported thereon, relative to the cartridge body 22110.

As a staple driver 22130 is contacted by a sled ramp, the staple driver 22130 may roll distally or, in other words, the proximal end of the staple driver 22130 may lift upwardly above the distal end. The staple cartridge assembly 22100 comprises a plurality of surfaces 22120 and a plurality of interaction surfaces 22121, 22122 between the staple driver 22130 and the cartridge body 22110. The surfaces 22120 are sized and configured so as to reduce and/or eliminate contact between the cartridge body 22110 and the staple driver 22130 at the surfaces 22120. The interaction surfaces 22121, however, are where the staple driver 22130 is configured to roll against, and be supported by, the cartridge body 22110 while the staple driver 22130 is being lifted upwardly during the staple firing stroke. The interaction surfaces 22122 are also configured to engage the cartridge body 22110 and provide a roll-countering force to counter the reaction force produced at the interaction surfaces 22121. Notably, the interaction surfaces 22121 and 22122 between the staple driver 22130 and the cartridge body 22110 are adjacent to a center plane CP of the staple driver 22130 whereas the surfaces 22120 are positioned further away from the center plane CP than the interaction surfaces 22120. Such an arrangement can reduce the roll of the staple drivers 22130 as the staple drivers 22130 are being lifted during the staple firing stroke.

As described above, a cartridge body of a staple cartridge assembly can comprise a unitary plastic body. In various embodiments, as described above, such a cartridge body can be manufactured using an injection molding process, for example. In at least one embodiment, a cartridge body can be manufactured using an insert molding process where a reinforcing material is at least partially encapsulated in the cartridge body. In at least one instance, the inner cartridge walls 22114, the intermediate cartridge wall pillars 22115, and the outer cartridge walls 22116 comprise material reinforcement molded into the structures themselves. In at least one instance, such reinforcement material can reduce deflection, collapsing, and/or twisting within the cartridge body. In at least one instance, the reinforcement material is oriented in a direction that is aligned with a high force vector and/or a direction that experiences a high degree of strain. In at least one instance, a reinforcement material is positioned along the sides of the longitudinal slot defined in the cartridge body that is configured to receive a firing driver.

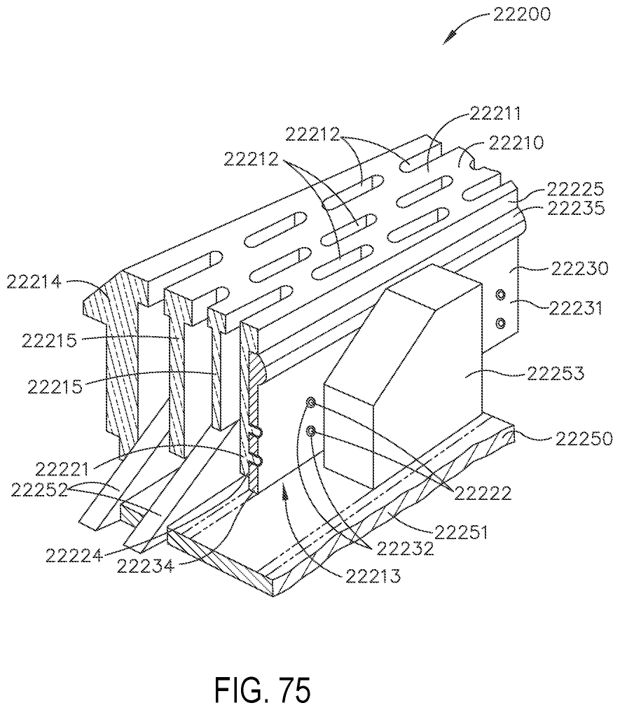

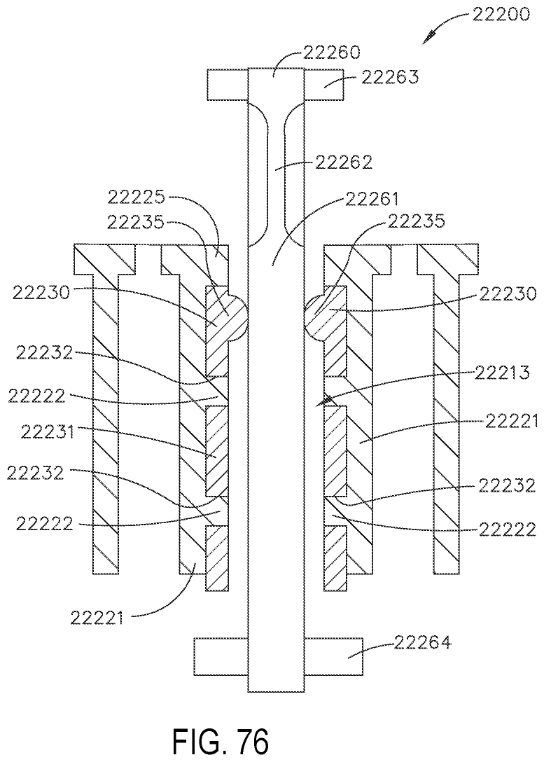

FIGS. 75 and 76 depict a surgical stapling assembly 22000 comprising a staple cartridge 22210 and a firing driver 22260 movable distally to fire the staple cartridge 22210, cammingly hold opposing jaws in a fully clamped position, and cut patient tissue captured between the jaws during a staple firing stroke, as described in greater detail further below. The staple cartridge 22210 comprises a cartridge body 22211 including staple cavities 22212 defined therein, a plurality of staples removably stored in the staple cavities 22212, and staple drivers movable to eject the staples from the staple cavities 22212 during the staple firing stroke. The staple cartridge assembly 22210 further comprises a sled 22250 movable through the cartridge body 22211 by the firing driver 22260 during the staple firing stroke to sequentially lift the staple drivers and staples toward an anvil jaw positioned opposite the staple cartridge 22200. In alternative embodiments, the staples of a staple cartridge are configured to be directly driven by a sled. In such embodiments, the staple cartridge does not have staple drivers.

The cartridge body 22211 further comprises a longitudinal slot 22213 configured to receive the firing driver 22260 and at least a portion of the sled 22250 during the staple firing stroke. The cartridge body 22211 also comprises a plurality of longitudinally-extending cartridge walls including outer cartridge walls 22214, intermediate cartridge walls 22215, and inner cartridge walls 22221. When the staple cartridge 22200 is installed in a cartridge channel jaw of a surgical stapling instrument, the outer cartridge walls 22214 are supported by walls of the cartridge channel jaw. The intermediate cartridge walls 22215 are positioned between the outer cartridge walls 22214 and the inner cartridge walls 22221. The inner cartridge walls 22221 are positioned adjacent the longitudinal slot 22213. The cartridge walls 22214, 22215, 22221 define longitudinally-extending cavities therebetween that are configured to receive sled ramps 22252 of the sled 22250 during the staple firing stroke.

The sled 22250 comprises a bottom portion 22251 and a central portion 22253 extending from the bottom portion 22251. The central portion 22253 is configured to traverse the longitudinal slot 22213 during the staple firing stroke. The sled ramps 22252 also extend from the bottom portion 22251 and are configured to lift the staple drivers as the sled 22250 is translated through the cartridge body 22211 during the staple firing stroke. It should be appreciated that the view presented in FIG. 75 is a partial cross-sectional view of the surgical stapling assembly 22200 and that the surgical stapling assembly 22200 is laterally-symmetric relative to a longitudinal axis defined by the longitudinal slot 22213 such that the staple cartridge 22210 comprises longitudinal rows of cavities and cartridge walls on the opposite sides of the longitudinal slot 22213. Likewise, the sled 22250 comprises sled ramps 22252 on opposing sides thereof that are configured to eject staples on the opposite side of the longitudinal slot 22213.

Further to the above, the firing driver 22260 comprises a distal I-beam head comprising a body portion 22261, a cutting edge 22262 defined on a distal end of the body portion 22261 to cut patient tissue captured between opposing jaws, and jaw cams 22263, 22264 configured to cammingly hold the opposing jaws in a fully-clamped configuration during the staple firing stroke. Also further to the above, the staple cartridge 22210 can be subject to clamping loads, for instance, which may encourage the cartridge body 22211 to deflect. To prevent or reduce such deflection, the staple cartridge 22210 further comprises a support insert 22230 that at least partially defines the longitudinal slot 22213. In at least one instance, the support insert 22230 counters inward deflection of the inner cartridge wall 22221. In at least one instance, the support insert 22230 is comprised of metal, for example. In at least one instance, the support insert 22230 is molded within the inner cartridge wall 22221. In at least one instance, the support insert 22230 is snap-fit with and/or press-fit to the cartridge body 22211 to and/or attached to the cartridge body 22211 using at least one adhesive, for example.

The support insert 22230 comprises a secondary wall structure adjacent the inner cartridge wall 22221. The support insert 22230 comprises a primary wall portion 22231 and an upper support rail 22235. The primary wall portion 22231 is adjacent the inner cartridge wall 22221 and the upper support rail 22235 is adjacent an upper ledge 22225 of the inner cartridge wall 22221. The support insert 22230 further comprises apertures 22232 defined therein configured to receive corresponding posts 22222 extending inwardly toward the longitudinal slot 22213 from the inner cartridge wall 22221. The posts 22222 and apertures 22232 are configured to align and/or hold the support insert 22230 to the inner cartridge wall 22221. The upper support rail 22235 extends laterally inwardly into the longitudinal slot 22213 such that the sled 22250 and/or firing driver 22260 are guided by the support rail 22235 during the staple firing stroke and, concurrently, the sled 22250 and/or firing driver 22260 provide support to the cartridge body 22211.

Further to the above, the staple cartridge 22210 comprises two support inserts 22230—one on each side of the longitudinal slot 22213—with each having an inwardly-facing support rail 22235. The support rails 22235 extend along the entire length of the support inserts 22230 and are supported by the sled 22250 and/or firing driver 22260 throughout the entire length of the staple firing stroke. In other embodiments, the support rails 22235 do not extend along the entire length of the support inserts 22230. In at least one instance, the opposing support rails 22235 comprise longitudinal gaps defined therein such that the sled 22250 and/or firing driver 22260 are not in contact with the support rails 22235 during low-load portions of the staple firing stroke but are in contact with the support rails 22235 during high-load portions of the staple firing stroke. In at least one embodiment, the sled 22250 and firing driver 22260 are not engaged with the support rails 22235 during the initial 15 mm of the staple firing stroke but are engaged with the support rails 22235 distal to the initial 15 mm of the staple firing stroke, for example.

The primary wall portion 22231 further comprises a bottom 22234 extending below a bottom 22224 of the inner cartridge wall 22221 that can transfer loads to the cartridge channel jaw outside of the cartridge body 22211. Clamping loads can be transferred through the inner cartridge wall 22221, for example, through the posts 22222 and down to the bottom portion 22251 of the sled 22250. In at least one instance, a cartridge pan and/or a cartridge channel are positioned directly beneath the bottom portion 22251 of the sled 22250 such that the bottom portion 22251 slides against and is supported by the cartridge pan and/or cartridge channel. In such an instance, the clamping load can be transferred through the support insert 22230, through the bottom portion 22251 of the sled 22250, and into the cartridge channel and/or cartridge pan, for example.

In at least one instance, the surgical stapling assembly 22200 provides cartridge deflection support without introducing excessive frictional forces to the firing stroke. Controlling the contact points where clamping load can be countered can help reduce component wear and firing malfunctions, for example. As discussed herein, cartridge deflection, collapsing, and/or twisting can cause staples to be ejected out of alignment with corresponding forming pockets defined in the anvil jaw and thus cause malformed staples and possibly inadequately stapled tissue, for example. In at least one instance, support inserts are provided in specific portions of the firing stroke. In at least one instance, a support insert is positioned only in the proximal portion of the firing stroke and not the distal portion of the firing stroke.

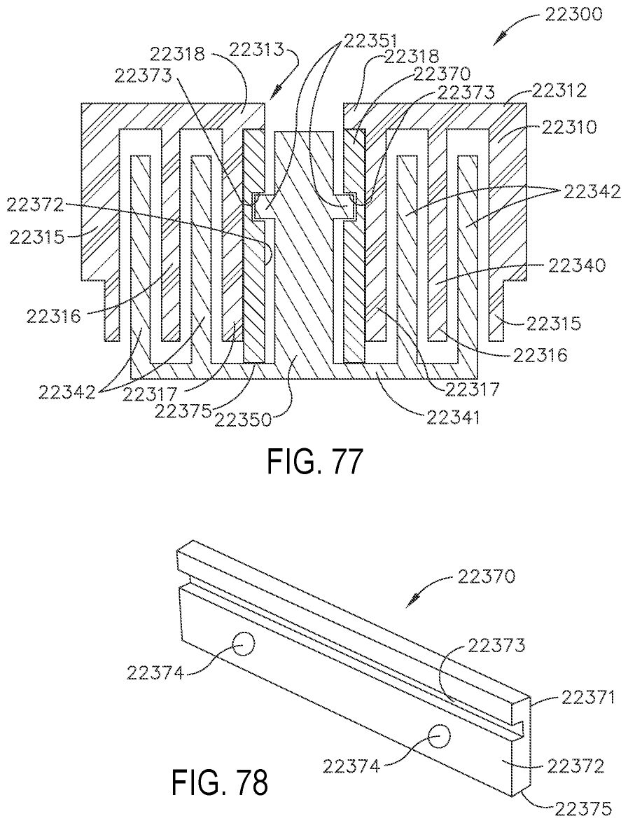

FIGS. 77 and 78 depict a staple cartridge 22300 for use with a surgical stapling instrument. The staple cartridge 22300 comprises a cartridge body 22310, a sled 22340 movable through the cartridge body 22310 to eject staples from the cartridge body 22310, and support inserts 22370 positioned within the cartridge body 22310. The cartridge body 22310 comprises a tissue-supporting deck 22312 against which tissue can be clamped and a longitudinal slot 22313 extending through the cartridge body 22310. The longitudinal slot 22313 is configured to receive at least a portion of the sled 22340 and a firing driver such as, for example, a distal I-beam head. The cartridge body 22310 further comprises a plurality of longitudinally-extending cartridge walls comprising outer cartridge walls 22315, intermediate cartridge walls 22316, and inner cartridge walls 22316. The sled 22340 comprises a bottom portion 22341 and ramps 22342 extending upwardly from the bottom portion 22341 into longitudinal cavities defined between the cartridge walls 22315, 22316, 22317 that sequentially lift and eject staples from the cartridge body 22310.

When the staple cartridge 22300 is seated in the cartridge channel of a surgical stapling instrument, the outer cartridge wall 22315 is supported by the walls of the cartridge channel. The sled 22340 further comprises a central rib 22350 positioned within the longitudinal slot 22313 such that the central rib 22350 translates within the longitudinal slot 22313 during a firing stroke. The sled 22340 is movable distally by a longitudinally-translatable firing driver such as a distal I-beam head, for example. In at least one instance, the sled 22340 is actuatable by a drive screw. In either event, the longitudinal slot 22313 is at least partially defined by support inserts 22370. The support inserts 22370 are positioned against the inner cartridge walls 22317 and ledges 22318 of the inner cartridge walls 22317. In at least one instance, the support inserts 22370 comprise a metal material, such as stainless steel, for example. In at least one instance, the support inserts 22370 are over molded onto the inner cartridge walls 22317.

Each support insert 22370 comprises an outer face 22371, an inner face 22372, and a longitudinally-extending channel 22373 defined in the inner face 22372. Each support insert 22370 further comprises a bottom support surface 22375 and fasteners 22374 configured to hold the support insert 22370 to the inner cartridge wall 22317. The fasteners 22374 may comprise any suitable type of fastener such as, for example, adhesive, screws, and/or rivets. In at least one instance, the fasteners 22374 comprise portions of the inner cartridge wall 22317 which are thermoplastically staked against the support insert 22370.

The support inserts 22370 are configured to transfer clamping loads from the cartridge body 22310 to the bottom portion 22341 of the sled 22340 and, in at least one instance, to an opposing support surface such as a staple cartridge-supporting surface of a cartridge channel, for example. The support inserts 22370 extend below the inner cartridge walls 22317 where the bottom support surfaces 22375 of the support inserts 22370 contact the top of the bottom portion 22341 of the sled 22340. In at least one embodiment, the bottom surfaces of the cartridge walls 22315, 22316, 22317 are sized and configured such that they cannot push downwardly on the sled 22340 when the staple cartridge 22300 is subject to load. In such embodiments, a large portion of the downward clamping force applied to the staple cartridge 22300 flows downwardly to the cartridge channel in and/or near the central or inner portion of the staple cartridge 22300.

The central rib 22350 of the sled 22340 further comprises lateral projections 22351. The lateral projections 22351 translate within the channels 22373 of the support inserts 22370 during the staple firing stroke. In various instances, the lateral projections 22351 of the sled 22340 engage the sidewalls of the channels 22373 which can align the sled 22340 relative to the cartridge body 22310 during the firing stroke. In at least one instance, engagement between the lateral projections 22351 and the sidewalls of the channels 22373 reduce lateral and vertical deflection of the cartridge body 22310 during the staple firing stroke. As the sled 22340 is pushed distally through the staple firing stroke, the bottom support surfaces 22375 of the support inserts 22370 are slidably supported on the bottom portion 22341 of the sled 22340. Clamping loads experienced by the cartridge body 22310 can be transferred through the bottom portion 22341 by the bottom support surfaces 22375 of the support inserts 22370. In addition to or in lieu of the above, the firing driver pushing the sled 22340 distally can have lateral projections 22351 that translate within channels 22373 and engage the sidewalls of the channels 22373, as described above. When both the sled 22340 and the firing driver both have such lateral projections, the lateral projections on the firing driver can help further distribute clamping loads along a height of an end effector and act as a central camming member in between an upper jaw-camming member and a lower jaw-camming member of the firing driver.

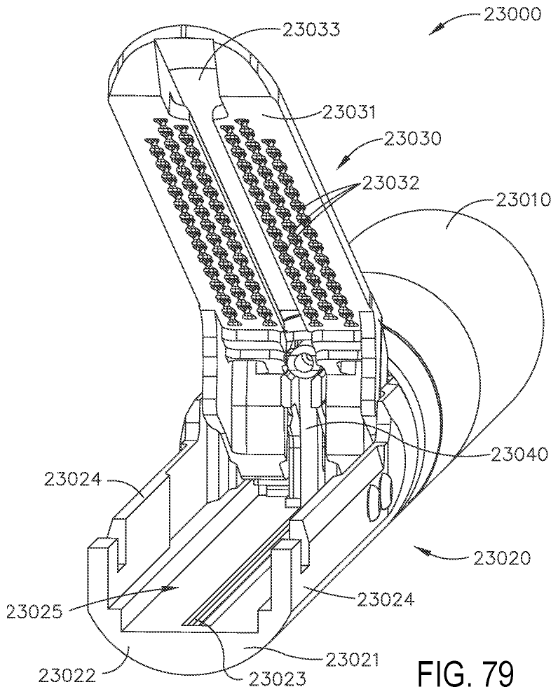

FIGS. 79-84 depict a surgical stapling assembly 23000 configured to clamp, cut, and staple patient tissue during a surgical stapling procedure. The surgical stapling assembly 23000 comprises a shaft 23010, a cartridge channel jaw 23020, and an anvil jaw 23030 movable relative to the cartridge channel jaw 23020 to clamp patient tissue therebetween. In alternative embodiments, the cartridge channel jaw 23020 is movable relative to the anvil jaw 23030. The surgical stapling assembly 23000 further comprises a staple cartridge 23050 configured to be installed into a cartridge channel 23021 of the cartridge channel jaw 23020. The surgical stapling assembly 23000 further comprises a firing driver 23040 actuatable, or movable distally, relative to the cartridge channel jaw 23020 and the anvil jaw 23030. In at least one instance, the firing driver 23040 comprises an I-beam head comprising opposing jaw-camming members that is movable distally to staple and cut patient tissue captured between the staple cartridge assembly 23050 and the anvil jaw 23030 during a staple firing stroke. In various embodiments, the surgical stapling assembly 23000 is coupled to any suitable actuation interface such as, for example, a surgical robot and/or a surgical instrument handle that is actuatable to control the operation of the surgical stapling assembly 23000.

The cartridge channel 23021 comprises a bottom portion 23022 and channel sidewalls 23024 extending upwardly from the bottom portion 23022 defining a channel cavity 23025 within which the staple cartridge 23050 is configured to be installed. The cartridge channel 23021 further comprises a longitudinal slot 23023 defined therein that is configured to receive at least a portion of the firing driver 23040 during the staple firing stroke. The anvil jaw 23030 comprises a tissue compression surface 23031, a plurality of forming pockets 23032, and a longitudinal slot 23033 configured to receive at least a portion of the firing driver 23040 during the staple firing stroke. Further to the above, the longitudinal slot 23023 of the cartridge jaw 23020 and the longitudinal slot 23033 of the anvil jaw 23030 are each configured to receive a jaw-camming member of the firing driver 23040, for example, during the staple firing stroke. The jaw-camming members are configured to hold the jaws 23020, 23030 in a clamped position during the firing stroke; however, embodiments are envisioned in which a firing driver does not comprise jaw-camming members.

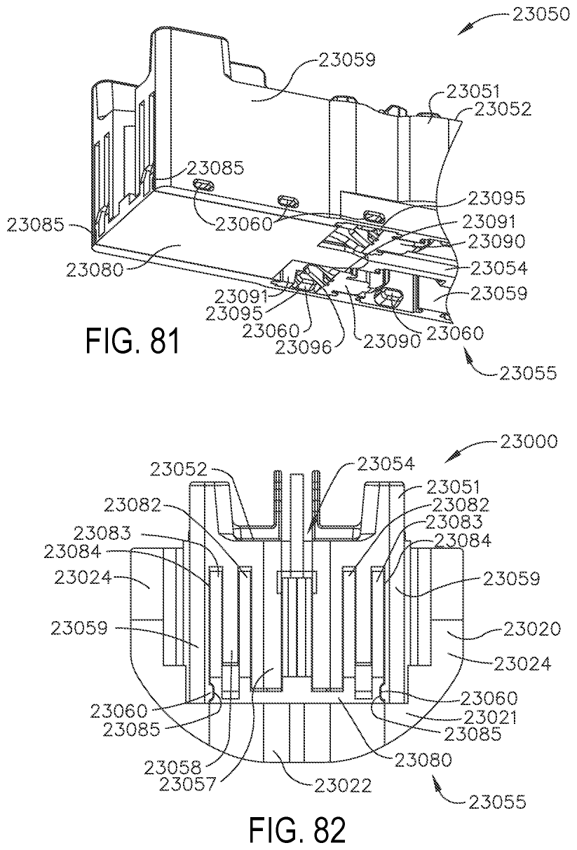

As discussed above, the staple cartridge assembly 23050 is configured to be installed in the cartridge channel jaw 23020. The staple cartridge 23050 comprises a cartridge body 23051, a sled 23080 movable from a proximal end 23055 of the cartridge body 23051 to a distal end 23056 of the cartridge body 23051 during a staple firing stroke, and a plurality of staple drivers 23090 liftable by the sled 23080 from an unfired position (FIG. 81) to a fired position to lift staples 23001 supported on the staple drivers 23090 toward the anvil jaw 23030 during the staple firing stroke. The staples 23001 are removably stored within the cartridge body 23051 and are configured to be formed by the forming pockets 23032 defined in the anvil jaw 23030. The cartridge body 23051 comprises a longitudinal slot 23054 configured to receive at least a portion of the firing driver 23040 during the staple firing stroke, a deck surface 23052 configured to support patient tissue thereon, and a plurality of staple cavities 23053 configured to removably store the staples 23001 therein. The sled 23080 is actuatable through the cartridge body 23051 to cammingly lift the staple drivers 23090 relative to the cartridge body 23051 to eject the staples 23001 from the staple cavities 23053.

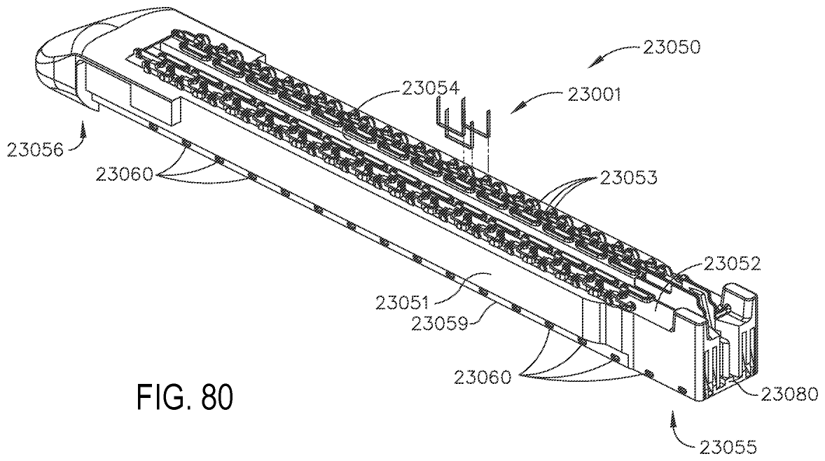

Referring to FIG. 80, the staple cartridge 23050 is pan-less. In other words, the staple cartridge 23050 does not comprise a pan that extends around the bottom of the cartridge body 23051 that prevents the staple drivers 23090 and/or the sled 23080 from falling out of the bottom of the cartridge body 23051. Discussed in greater detail below, the staple cartridge 23050 further comprises retention features, such as retention features 23060, for example, configured to prevent the staple drivers 23090 and the sled 23080 from falling out of the bottom of the cartridge body 23051. As also discussed in greater detail below, the retention features 23060 are also configured to guide the sled 23080 as the sled 23080 is moved distally through the cartridge body 23051 during the staple firing stroke.

Referring to FIG. 82, the cartridge body 23051 further comprises a plurality of cartridge walls comprising inner walls 23057 defining the longitudinal slot 23054, intermediate walls 23058, and outer walls 23059 that are positioned against and supported by the channel sidewalls 23024 when the staple cartridge 23050 is seated in the cartridge jaw 23020. The sled 23080 comprises a bottom portion 23081 and a central rib 23086 extending upwardly from the bottom portion 23081 that is configured to be received within the longitudinal slot 23054 of the cartridge body 23051. The central rib 23086 comprises a knife 23087 configured to cut tissue as the sled 23080 is moved distally during the staple firing stroke. The sled 23080 further comprises inner ramp wedges 23082 extending upwardly from the bottom portion 23081 and outer ramp wedges 23083 extending upwardly from the bottom portion 23081. The ramp wedges 23082, 23083 are configured to lift the staple drivers 23090 relative to the cartridge body 23051 during the staple firing stroke to drive the staples supported thereon. Discussed in greater detail below, the outer ramped wedges comprise outer walls 23084 including retention slots, notches, and/or grooves, 23085 defined therein configured to guide the sled 23080 within the cartridge body 23051 during the staple firing stroke and hold the sled 23080 in the cartridge body 23051.

As discussed above, the retention features 23060 are configured to prevent the staple drivers 23090 and the sled 23080 from falling out of the bottom of the cartridge body 23051. The retention features 23080 are arranged in a longitudinal array on each lateral side of the cartridge body 23051. Referring to FIG. 81, each side of the cartridge body 23051 has at least one retention feature 23090 configured to releasably hold the sled 23080 in its proximal unfired position. When the sled 23080 is pushed distally by the firing driver, the sled 23080 releases from the proximal-most retention features 23090 so that the sled 23080 can be advanced distally through the staple firing stroke. Each other retention feature 23060 is configured to hold a staple driver 23090 in an unlifted position and, as the sled 23080 passes thereby, release the staple driver 23090 so that the sled 23080 can lift the staple driver 23090 into a fired position. Such retention features 23060 also hold and guide the sled 23080 in the cartridge body 23051. Each retention feature 23060 comprises at least one protrusion extending inwardly toward the longitudinal slot 23054 from an outer wall 23059; however, the retention features 23060 can comprise any suitable configuration. Referring to FIG. 84, the retention features 23060 extend into notches 23085 defined in the sled 23080 which prevent the sled 23080 from falling out of the bottom of the cartridge body 23051. As can be seen in FIG. 84, the notches 23085 extend the entire longitudinal length of the sled 23080 so as to allow the sled 23080 to simultaneously engage two or more retention features 23060 at all points during the staple firing stroke, as discussed in greater detail below.

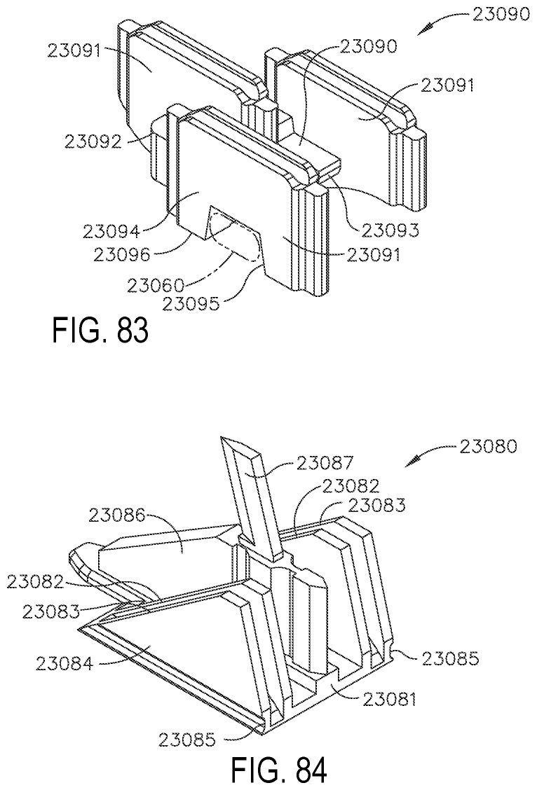

Referring to FIG. 83, each staple driver 23090 comprises a plurality of staple support columns 23091 configured to support the staples 23001 thereon and web portions 23092 connecting the staple support columns 23091. Each support column 23091 comprises a seat, or cradle, that receives a base of a staple 23001. Each staple driver 23090 further comprises at least one lift cam surface 23093 configured to be engaged by the ramp wedges 23082, 23083 of the sled 23080 during the staple firing stroke to lift the staple driver 23090 relative to the cartridge body 23051. Each staple driver 23090 comprises an outer driver wall 23094 defined by the staple support column 23091 nearest the outer wall 23059 of the cartridge body 23051. A notch 23095 is defined in the outer driver wall 23094 and is configured to receive a retention feature 23060 therein when the staple driver 23090 is in its unfired, or unlifted, position such that the retention feature 23060 holds the staple driver 23090 in the cartridge body 23051 without a cartridge pan extending under the bottom of the cartridge body 23051. As the staple drivers 23090 are lifted upwardly by the sled 23080, the staple drivers 23090 disengage from the retention features 23060.

Further to the above, referring again to FIG. 81, two retention features 23060 releasably hold the sled 23080 in its unfired position; however, any suitable number of retention features 23060 are contemplated for holding the sled 23080 in its unfired position. As can also be seen in FIG. 81, retention features 23060 extend inwardly from the outer walls 23059 into the staple cavities 23053. Such retention features 23060, as discussed above, are configured to hold the staple drivers 23090 in their unfired positions and support the sled 23080 during the staple firing stroke. In at least one instance, each staple driver 23090 is held in its unfired, unlifted position using two retention features 23060—one extending laterally inwardly toward the longitudinal slot 23054 from the outer wall 23059 and one extending laterally outwardly toward the outer wall 23059 from the inner wall 23057. In such instances, the retention feature 23060 extending laterally inwardly from the inner wall 23057 fits within a corresponding notch defined in an inner wall of a staple support column 23091 adjacent the inner wall 23057 of the cartridge body 23051.

As the sled 23080 is actuated through the staple firing stroke, the ramp wedges 23082, 23083 of the sled 23080 lift the staple drivers 23090 from their unfired, unlifted position to a fired, lifted position to eject the staples 23001 from the cartridge body 23051. As the ramp wedges 23082, 23083 lift the staple drivers 23090, the notches 23085 defined in the sled 23080 are engaged with one or more retention features 23060 such that the retention features 23060 guide the sled 23080 through the firing stroke and hold the sled 23080 in the cartridge body 23051. In at least one instance, at least two retention features 23060 are engaged with the sled 23080 for any given position of the sled 23080 along its firing stroke. In at least one instance, more than two retention features 23060 are engaged with the sled 23080 for any given position of the sled 23080. In such instances, the sled 23080 can simultaneously lift two or more staples 23001 in each outer longitudinal row of staple cavities and can be concurrently engaged with the retention features 23060 associated with the staple drivers 23090 being driven, or lifted.

Further to the above, the retention features 23060 can be created using any suitable process. In at least one instance, referring to FIG. 81, the creation of the retention features 23060 in the cartridge body 23051 creates divots, recesses, and/or dimples in the outside lateral surfaces of the cartridge body 23051 that, as discussed below, can be used to advantageous effect. For instance, the channel sidewalls 23024 of the cartridge jaw comprise bumps that that are aligned with, and received within, the divots when the staple cartridge 23050 is installed in the channel cavity 23025 that releasably hold the staple cartridge 23050 in the channel cavity 23025. Moreover, the bumps extending from the channel sidewalls 23024 support the retention features 23060 which, among other things, assist in preventing the staple drivers 23090 from releasing prematurely. With or without such bumps, the width of the staple cartridge 23050 can be configured such that the retention features 23060 are at least partially squeezed inwardly by the channel sidewalls 23024 upon the installation of the staple cartridge 23050 into the cartridge channel 23021.

In various instances, the retention features 23060 are formed using a thermoplastic heat staking process. In at least one instance, the retention features 23060 are formed after the installation of the staple drivers 23090 into the cartridge body 23051. In at least one instance, the retention features 23060 are formed before the staple drivers 23090 are installed into the cartridge body 23051. In at least one such instance, the staple drivers 23090 are pressed up into the staple cavities 23053 from the bottom of the cartridge body 23051 and the retention features 23060 flex laterally outwardly until the staple drivers 23090 are fully installed in the cartridge body 23051. At such point, the retention features 23060 can reassume their original position, or at least resiliently return toward their original position, to hold the staple drivers 23090 in the cartridge body 23051.

In at least one instance, such retention features can help reduce the tendency of a sled and/or staple drivers to become dislodged from the staple cartridge prior to the staple cartridge being installed in the stapling instrument. When a fired staple cartridge assembly is uninstalled from the cartridge channel, moreover, the retention features can prevent the already-fired staple drivers from falling out of the bottom of the cartridge body. Additionally, in at least one instance, the sled of the staple cartridge is configured to be slid into the proximal end of the cartridge body. In such instances, the notches defined in the sled and corresponding retention features align the sled relative to the cartridge body when the sled is installed into the cartridge body.

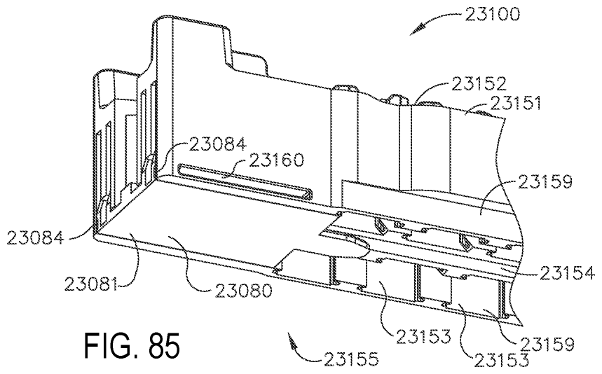

FIG. 85 is a partial perspective view of a staple cartridge 23100 comprising a cartridge body 23151 and the sled 23080. The cartridge body 23151 comprises a deck 23152, a plurality of staple cavities 23153, and a longitudinal slot 23154. The cartridge body 23151 further comprises a proximal end 23155 and outer cartridge body walls 23159. The outer cartridge body walls 23159 comprise elongate retention features, or rails, 23160 extending laterally inwardly toward the longitudinal slot 23154. The notches 23085 of the sled 23080 receive the retention rails 23160 in the unfired, or unadvanced, position of the sled 23080. In at least one instance, the retention rails 23160 are formed using a thermoplastic heat staking process, for example, that pushes inwardly on the outside walls 23159 of the cartridge body 23151. In any event, the retention rails 23160 support the sled 23080 in its proximal unfired position and also guide the sled 23080 during the initial part of the staple firing stroke. In various embodiments, the cartridge body 23151 further comprises retention features 23060 that releasably hold the staple drivers in their unfired positions and support and guide the sled 23080 during the remainder of the staple firing stroke.

In various instances, a sled of a staple cartridge may become dislodged when the staple cartridge is shipped and/or handled prior to being fully installed in a cartridge channel of a stapling instrument. For instance, the sled may be bumped distally when being handled by a technician and/or bumped distally against a firing member when the staple cartridge is installed proximally into a stapling instrument. In at least one instance, as described in greater detail below, a staple cartridge is provided which prevents the sled from being moved out of its unfired position until the staple cartridge is fully seated in a cartridge channel.

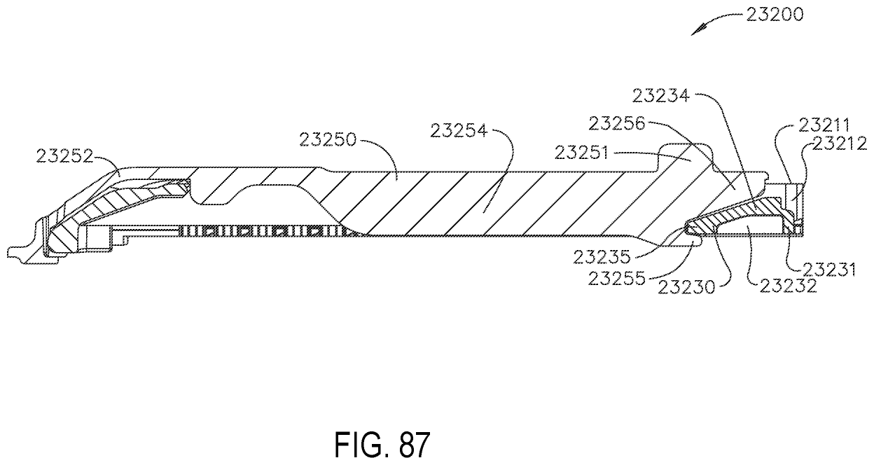

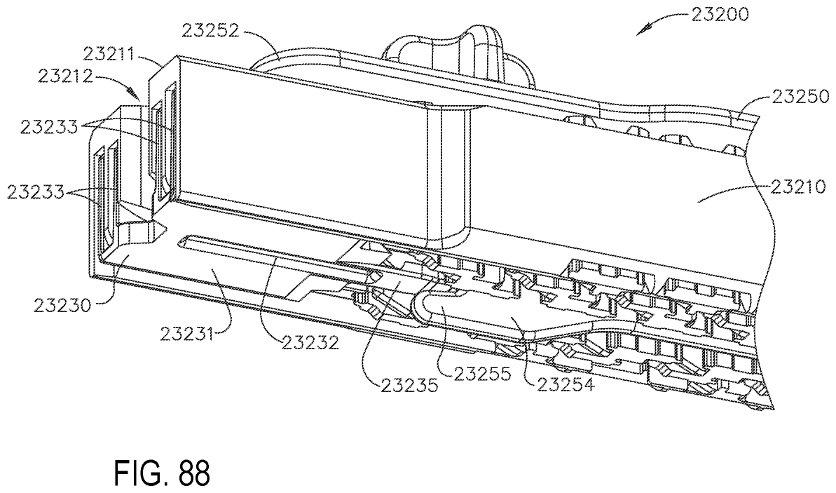

FIGS. 87-89 depict a staple cartridge 23200 for use with a surgical stapling assembly. In at least one instance, the staple cartridge 23200 is configured to be installed in a cartridge channel jaw of a surgical stapling assembly. The staple cartridge 23200 comprises a cartridge body 23210 and a plurality of staples removably stored in the cartridge body 23210 that are ejected from the cartridge body 23210 by a sled 23230 as the sled 23230 is translated through the cartridge body 23210 from an unfired position to a fired position during a staple firing stroke. The cartridge body 23210 comprises a cartridge deck 23211 and a longitudinal slot 23212 defined in the cartridge deck 23211 that is configured to receive at least a portion of the sled 23230 as well as a portion of a firing driver which pushes the sled 23230 distally during the staple firing stroke.

The sled 23230 comprises a bottom portion 23231 and a plurality of ramped wedges 23233 extending upwardly into the cartridge body 23210 from the bottom portion 23231. The ramped wedges 23233 are configured to lift staple drivers and eject staples from the cartridge body 23210 during the staple firing stroke. The sled 23230 further comprises a central rib 23234 configured to be received within the longitudinal slot 23212 and a distal nose portion 23235.

The staple cartridge 23200 further comprises a staple retainer 23250 that is installed onto, or attached to, the cartridge body 23210 prior to the staple cartridge 23200 being shipped to serve as a sterile barrier to staples positioned in the cartridge body 23210 and to prevent the staples from falling out of the top of the cartridge body 23210. The staple retainer 23250 is configured to be removed from the cartridge body 23210 prior to firing the staples of the staple cartridge 23200. In at least one instance, the staple retainer 23250 is removed after fully installing the staple cartridge 23200 in a cartridge channel jaw. In such instances, a clinician can push downwardly on the staple retainer 23250 to seat the staple cartridge 23200 in the cartridge channel jaw. In at least one instance, the staple retainer 23250 is removed prior to the staple cartridge 23200 being installed into a cartridge channel.

The staple retainer 23250 is positioned against the cartridge deck 23211 and comprises a proximal end 23251, a distal end 23252, and retention arms 23253 configured to releasably hold the staple retainer 23250 to the cartridge body 23210. The staple retainer 23250 further comprises a central fin 23254 extending down into the longitudinal slot 23212. The central fin 23254 comprises a proximal hook 23255 configured to engage the distal nose portion 23235 of the sled 23230 such that the staple retainer 23250 holds the sled 23230 in its unfired position and prevents the sled 23230 from falling out of the bottom of the cartridge body 23210. The central fin 23254 further comprises an upper ramped edge 23256 positioned adjacent the central rib 23234 that prevents the sled 23230 from being moved distally. In at least one instance, a user can pry the staple retainer 23250 off of the cartridge body 23210 by lifting on the distal end 23252 of the staple retainer 23250 and rotating the staple retainer 23250 relative to the cartridge body 23210 and the sled 23230. This rotation can allow the staple retainer 23250 to disengage the distal nose portion 23235 of the sled 23230 so as to not accidentally disturb the position of the sled 23230 during removal of the staple retainer 23250. In at least one instance, the user can pull the staple retainer 23250 distally relative to the sled 23230 when removing the staple retainer 23250 so as to not disturb the position of the sled 23230.

Regardless of the manner in which the staple retainer 23250 is detached from the cartridge body 23210, the staple retainer 23250 is configured to positively retain the sled 23230 in its unfired position by preventing vertical and longitudinal movement of the sled 23230 relative to the cartridge body 23210. Referring to FIG. 87, the sled 23230 comprises a retention slot, or recess, 23232 defined in the bottom thereof and the proximal hook 23255 is configured to be received within the retention slot 23232 when the staple retainer 23250 is attached to the cartridge body 23210. Such an arrangement can also prevent inadvertent proximal movement of the sled 23230 relative to the cartridge body 23210. In at least one instance, the proximal hook 23255 engages the sled 23230 in a snap-fit manner.

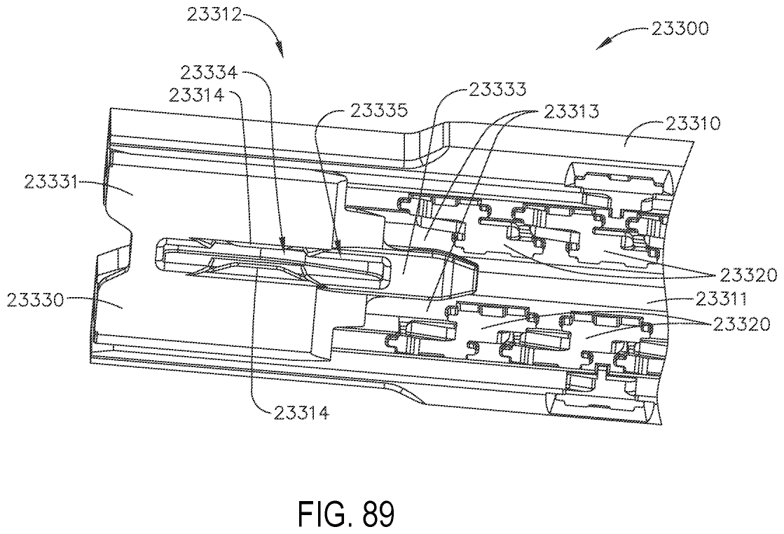

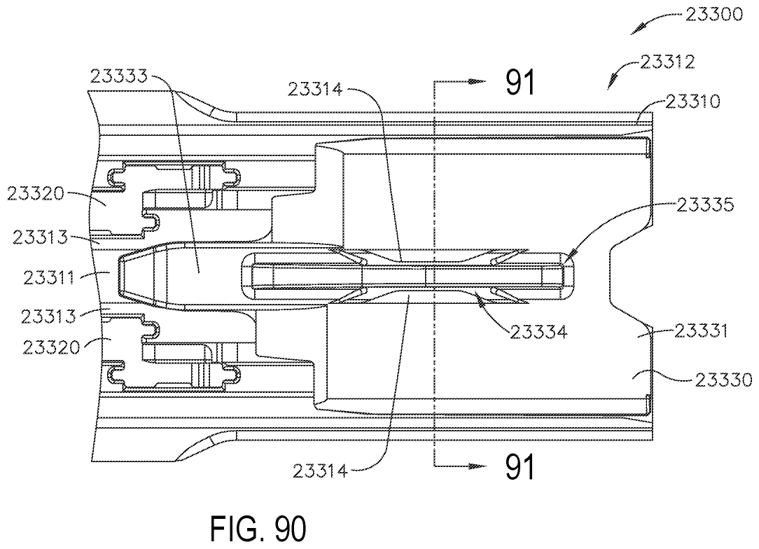

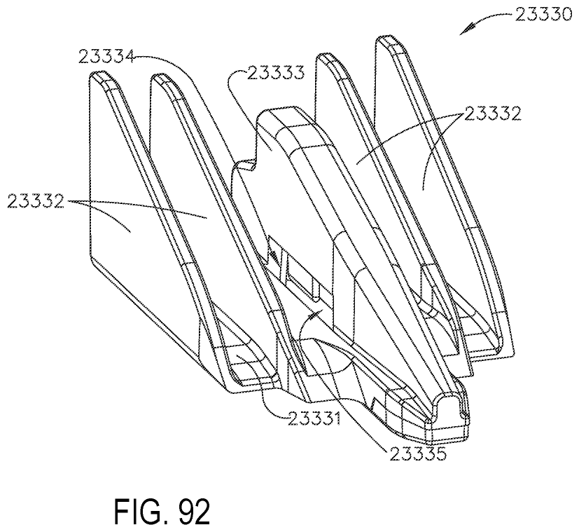

FIGS. 89-92 depict a staple cartridge 23300 that is configured to be installed in a cartridge channel of a surgical stapling assembly. The staple cartridge 23300 comprises a cartridge body 23310, a plurality of staple drivers 23320, and a plurality of staples configured to be ejected from the cartridge body 23310 by the staple drivers 23320 during a staple firing stroke. The staple cartridge 23300 further comprises a sled 23330 movable distally through the cartridge body 23310 from an unfired position (illustrated in FIGS. 89-92) to a fired position during the staple firing stroke to lift the staple drivers 23320 and eject the staples.

The cartridge body 23310 comprises a longitudinal slot 23311 configured to receive at least a portion of a firing driver therein. The cartridge body 23310 further comprises a proximal end 23312 within which the sled 23330 is positioned in an unfired configuration and inner cartridge walls 23313 defining the longitudinal slot 23311. The inner cartridge walls 23313 each comprise a retention feature 23314 extending laterally inwardly into the longitudinal slot 23311 to prevent the sled 23330 from falling out of the bottom of the cartridge body 23310 and from moving out of the unfired position prior to being fired.

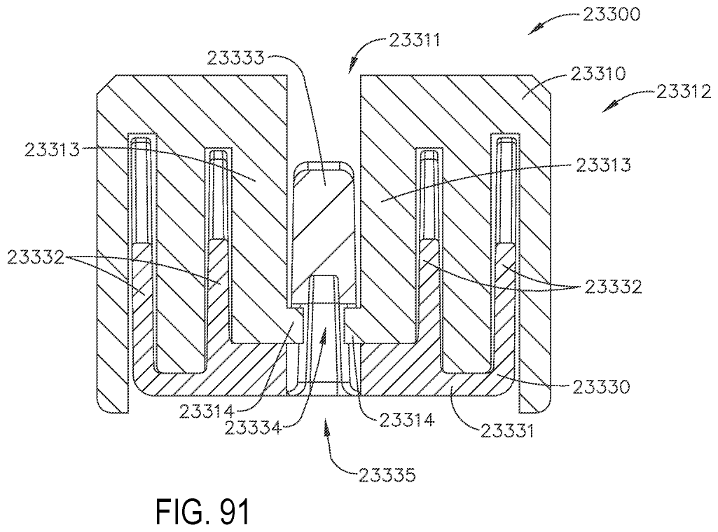

The sled 23330 comprises a bottom portion 23331 and ramped wedges 23332 extending upwardly from the bottom portion 23331 and into the cartridge body 23310. The ramped wedges 23332 are configured to lift the staple drivers 23320 to eject the staples supported thereon as the sled 23330 is moved distally through the cartridge body 23310 during the staple firing stroke. The sled 23330 further comprises a central portion 23333 that moves within the longitudinal slot 23311 of the cartridge body 23310 as the sled 23330 is moved distally. The cartridge body 23310 further comprises retention features, or shoulders, 23314 that extend inwardly toward the central portion 23333 of the sled 23330 that releasably hold the sled 23330 in its proximal unfired position. More specifically, the retention features 23314 extend into retention cavities 23334 defined in the central portion 23333 of the sled 23330 when the sled 23330 is in its unfired position. As can be seen in FIG. 91, the retention features 23314 comprise inwardly-extending ledges extending from the inner cartridge walls 23313. In various instances, the retention features 23314 are snap-fit, friction-fit, and/or press-fit into the retention cavities 23334, for example. When a firing driver of a surgical stapling assembly is advanced distally into contact with the sled 23330 at the beginning of the staple firing stroke, the firing driver pushes the sled 23330 distally out of engagement with the retention features 23334.

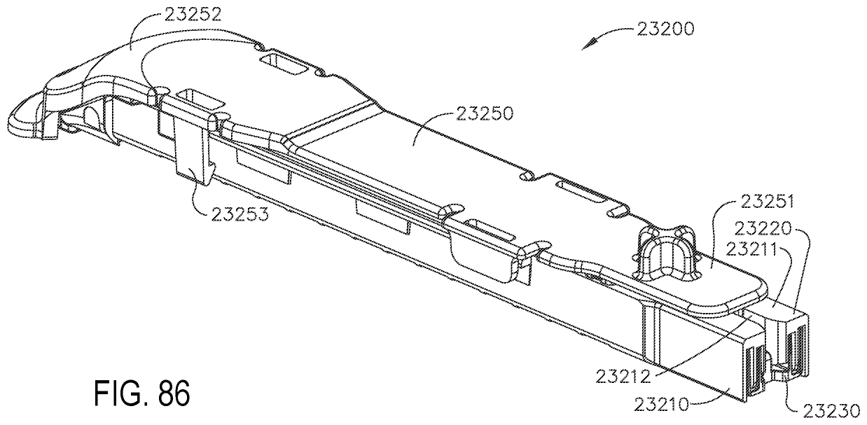

As discussed above, referring again to FIG. 86, a retainer can be removably attached to a cartridge body of a staple cartridge that, among other things, prevents a sled from being accidentally moved within the cartridge body while the staple cartridge is being shipped, handled, and/or installed in a stapling instrument. In the event that the sled is moved distally accidentally, for instance, the sled may inadvertently eject, or at least partially eject, some of the proximal-most staples stored in the staple cartridge. Moreover, in various embodiments, the sled may unlock the staple firing drive of a stapling instrument when the staple cartridge is installed in the stapling instrument and the sled is in its proximal unfired position. If, however, the sled is not in its unfired position when the staple cartridge is installed in the stapling instrument, in such embodiments, the sled may not unlock the firing drive and the stapling instrument may stay in a locked state until the staple cartridge is replaced. The entire disclosure of U.S. Pat. No. 6,988,649, entitled SURGICAL STAPLING INSTRUMENT HAVING A SPENT CARTRIDGE LOCKOUT, which issued on Jun. 24, 2006, is incorporated by reference herein.

In various alternative embodiments, the sled 23330 is movable from a shipped position to an unfired, ready-to-fire position when a staple retainer is removed from the staple cartridge. In at least one such embodiment, the retention features 23314 of the cartridge body 23310 are engaged with the sled 23330 when the sled 23330 is in the shipped position. When the staple retainer is lifted away from the cartridge body 23310, the staple retainer holds onto the sled 23330 and pulls the sled 23330 distally out of its shipped position into an unfired ready-to-fire position. In effect, the staple retainer pulls the sled 23330 out of engagement with the retention features 23314. In at least one instance, the retention features 23314 are configured to be broken by the staple retainer so as to place the sled 23330 in the ready to fire position. In at least one instance, the act of breaking the retention features 23314 repositions the sled 23330 from its shipped position to another relative longitudinal position where the sled 23330 is ready to be fired. In various embodiments, the cartridge body 23310 comprises a stop that is contacted by the sled 23330 when the sled 23330 is pulled distally which stops the distal movement of the sled 23330.

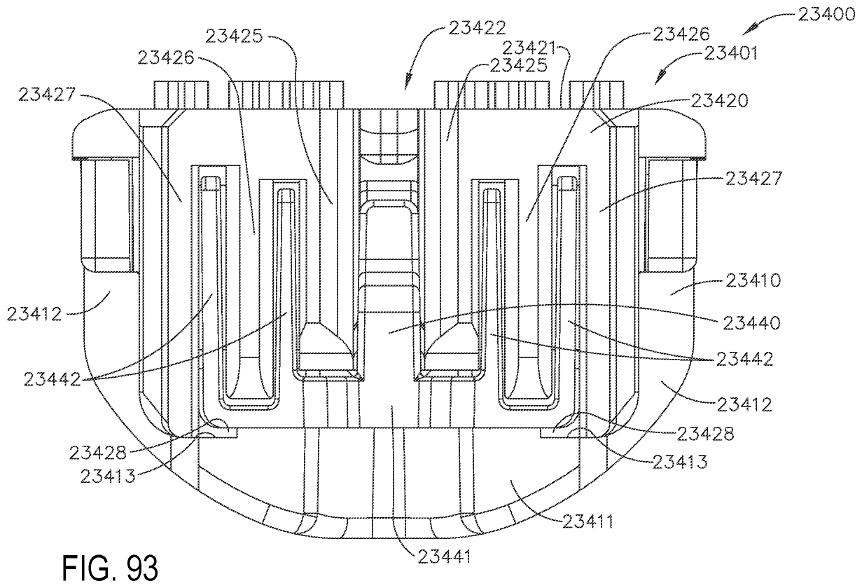

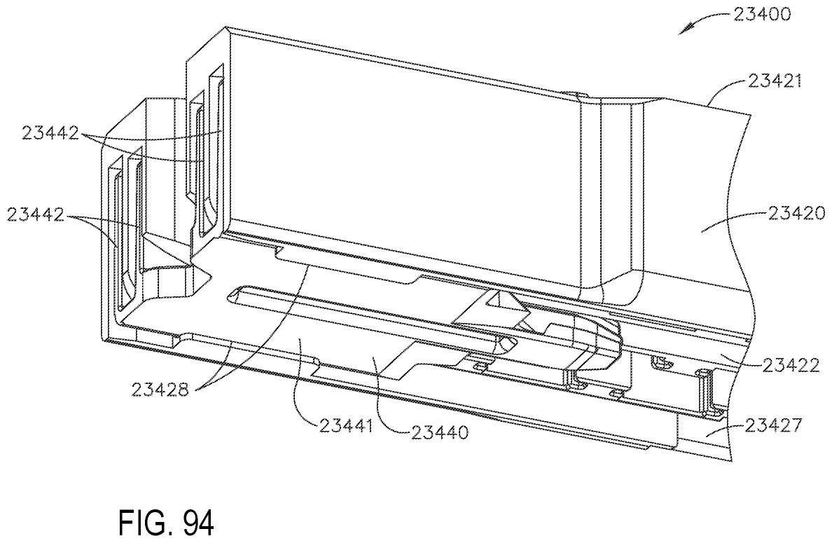

FIGS. 93 and 94 depict a surgical stapling assembly 23400 comprising a staple cartridge 23401 and a cartridge channel 23410 within which the staple cartridge 23401 is configured to be installed. The cartridge channel 23410 comprises a bottom portion 23411 and channel sidewalls 23412 extending upwardly from the bottom portion 23411. The staple cartridge 23401 comprises a cartridge body 23420, staple drivers, staples, and a sled 23440 movable through the cartridge body 23420 from an unfired position to a fired position during a staple firing stroke. The sled 23440 comprises a bottom portion 23441 and sled ramps 23442 extending upwardly into the cartridge body 23420 that are configured to lift the staple drivers during the staple firing stroke to eject the staples from the cartridge body 23420.

The cartridge body 23420 comprises a deck 23421 and a longitudinal slot 23422 configured to receive at least a portion of a firing driver therein during the staple firing stroke. The cartridge body 23420 comprises inner cartridge walls 23425 defining the longitudinal slot 23422, intermediate cartridge walls 23426, and outer cartridge walls 23427 that are positioned adjacent to and supported by the channel sidewalls 23412 when the staple cartridge 23401 is installed in the cartridge channel 23410. In at least one instance, the staple cartridge 23401 does not comprise a pan to hold drivers, staples, and/or the sled 23440 in the cartridge body 23420 when the staple cartridge assembly 23401 is not installed in the cartridge channel 23410. The outer cartridge walls 23427 each comprise a ledge tab 23428 extending laterally inwardly toward the longitudinal slot 23422 configured to prevent the sled 23440 from falling out of the bottom of the cartridge body 23420. The cartridge channel 23410 comprises notches 23413 defined therein that are configured to receive the ledge tabs 23428 when the staple cartridge 23401 is seated in the cartridge channel 23410. The ledge tabs 23428 of the staple cartridge 23401 are closely received within the notches 23413 of the cartridge channel 23410 and are restrained from moving horizontally, longitudinally, and downwardly with respect to the cartridge channel 23410 which can prevent or at least limit relative movement between the staple cartridge 23401 and the cartridge channel 23410.

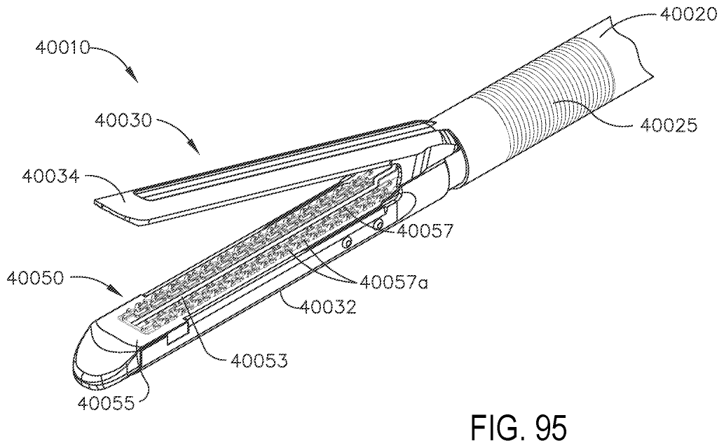

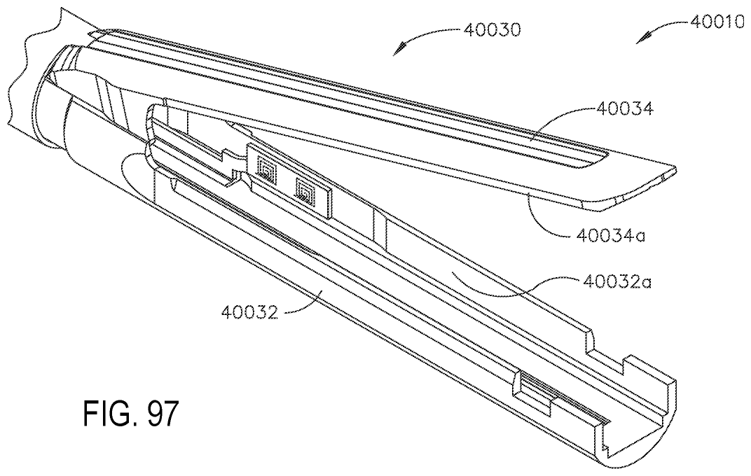

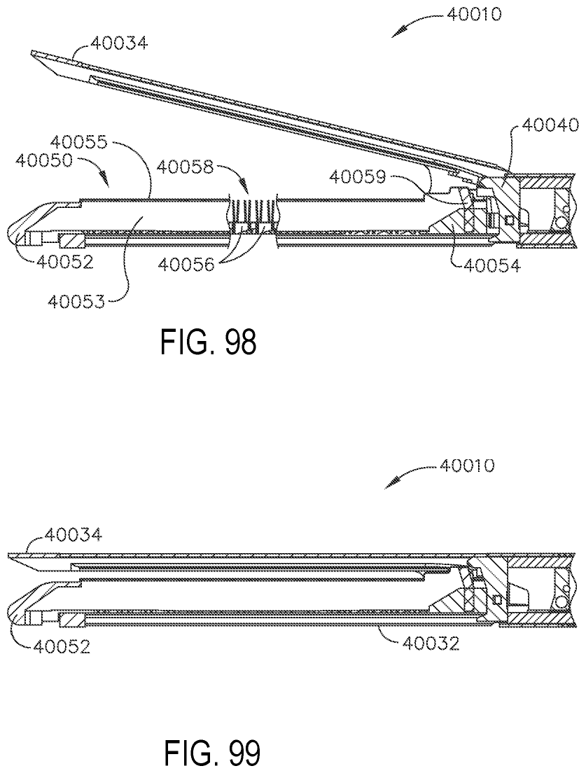

FIGS. 95-99 illustrate a stapling attachment 40010 for use with a surgical system. The stapling attachment 40010 comprises a shaft 40020 and an end effector 40030 extending from the shaft 40020. The stapling attachment 40010 further comprise an articulation joint 40025 positioned intermediate the end effector 40030 and the shaft 40020. The articulation joint 40025 is configured to permit the end effector 40030 to rotate relative to the shaft 40020 about the articulation joint 40025. The end effector 40030 comprises a first jaw 40032 and a second jaw 40034 movable relative to the first jaw 40032 between an open position (FIG. 98) and a closed position (FIG. 99) to capture the tissue of a patient therebetween. In at least one other embodiment, the second jaw 40034 is movable relative to the first jaw 40032. Referring to FIG. 97, the first jaw 40032 comprises an elongate channel 40032a and the second jaw 40034 comprises an anvil 40034a. The elongate channel 40032a of the first jaw 40032 is configured to receive a replaceable staple cartridge 40050 therein that can be readily removed during a surgical procedure and replaced with a new staple cartridge; however, other embodiments are envisioned in which the first jaw 40032 comprises a staple cartridge that is not replaceable. Referring primarily to FIG. 98, the staple cartridge 40050 comprises a cartridge body 40052, a sled 40054, a plurality of staple drivers 40056, and staples 40058 supported by the staple drivers 40056.

Further to the above, the cartridge body 40052 includes a longitudinal slot 40053 and a deck 40055 having a proximal end 40055a and a distal end 40055b. The longitudinal slot 40053 extends from the proximal end 40055a toward the distal end 40055b. The cartridge body 40052 further comprises a plurality of staple cavities 40057 defined in the cartridge body 40405. The staple cavities 40057 define a plurality of staple cavity openings 40057a defined in the deck 40055 of the cartridge body 40052. Referring primarily to FIG. 95, three rows of staple cavities 40057 are positioned on a first side of the longitudinal slot 40053 and three rows of staple cavities 40407 are positioned on a second side of the longitudinal slot 40053 opposite the first side. However, other arrangements of staple cavities and staples are contemplated. The staple cartridge 40050 further comprises lateral protrusions 40051 extending from the sides of the cartridge body 40052. The lateral protrusions 40051 are configured to be received in distal cutouts 40033 defined in the first jaw 40032 which can control the position of the staple cartridge 40050 in the first jaw 40032. In at least one instance, the lateral protrusions 40051 and distal cutouts 40033, and/or other features of the staple cartridge 40050 and the first jaw 40032, are configured to permit the staple cartridge 40050 to be snap-fit into the first jaw 40032.

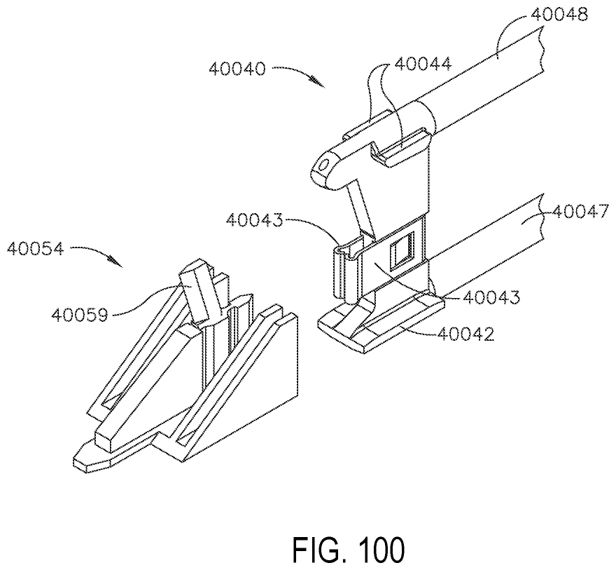

Referring now to FIGS. 98 and 100, the stapling attachment 40010 further comprises a firing driver 40040 movable relative to the end effector 40030 from a proximal position to a distal position during a firing stroke. The firing driver 40040 is configured to advance the sled 40054 relative to the staple cartridge 40050 from a proximal unfired position to a distal fired position during the firing stroke. The firing driver 40040 comprises a first cam 40042 configured to engage the first jaw 40032 during the firing stroke and second cams 40044 configured to engage the second jaw 40034 during the firing stroke. The firing driver 40040 further comprises a first firing bar, or rod, 40047 extending proximally from the firing driver 40040 and a second firing bar, or rod, 40048 extending proximally from the firing driver 40040. The first firing bar 40047 is spaced apart from the second firing bar 40048. The first and second firing bars 40047,40048 are configured to transfer a firing motion from the surgical system to the firing driver 40040 to translate the firing driver 40040 distally during the staple firing stroke and then proximally during a retraction stroke. In various embodiments, the surgical system comprises an electric motor and a motor control circuit which moves the firing driver 40040 that is responsive to an input from a clinician and/or a robotic surgical system.

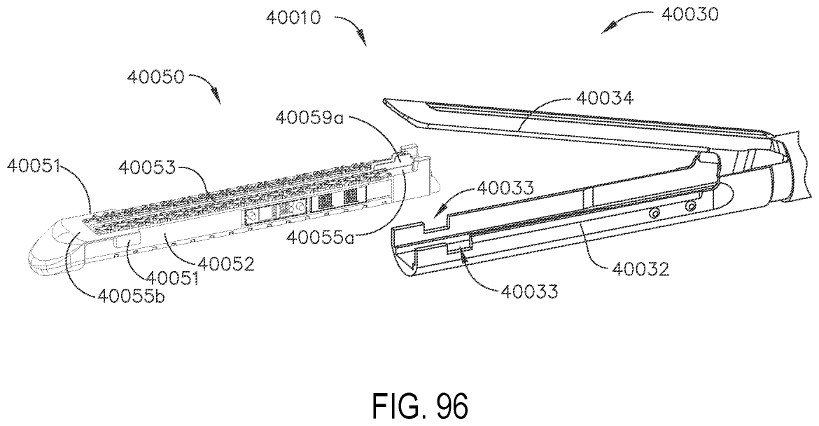

Further to the above, referring primarily to FIG. 100, the sled 40054 of the staple cartridge 40050 comprises a knife 40059. However, in at least one instance, the firing driver 40040 comprises the knife instead of the sled 40054. In any event, when the sled 40054 is in the proximal unfired position, as illustrated in FIG. 98, the knife 40059 is positioned within a proximal knife housing 40059a of the cartridge body 40052. As such, during shipping and assembly of the staple cartridge 40050, the knife 40059 of the sled 40054 is positioned within the knife housing 40059a and, thus, the chances of a user coming into contact with the knife 40059 when handling the staple cartridge 40050 is reduced (see FIG. 96).

In use, referring to FIG. 100, the firing driver 40040 is positioned proximal to—and not in contact with—the sled 40054 when the staple cartridge 40050 is seated in the first jaw 40032. The firing driver 40040 is advanced toward the sled 40054 at the outset of the firing stroke and engages the sled 40054 to advance the sled 40054 through the remainder of the firing stroke. More specifically, the firing driver 40040 comprises a pair of opposing lateral spring arms 40043 extending distally therefrom that engage, grip, and push the sled 40054 distally during the firing stroke. Moreover, the lateral spring arms 40043 couple the firing driver 40040 to the sled 40054 such that the sled 40054 is retractable by the firing driver 40040 during a retraction stroke. In at least one instance, the firing driver 40040 is configured to retract the sled 40054 proximally all the way into its original proximal unfired position. The staple cartridge 40050 further comprises a stop that is engaged by the sled 40054 when the sled 40054 is pulled back into its proximal unfired position by the firing driver 40040 such that, as the firing driver 40040 is retracted further back into its original proximal unfired position, the firing driver 40040 disengages from the sled 40054. In alternative embodiments, the firing driver 40040 and sled 40054 are one unitary structure which is movable relative to the end effector 40030. In such instances, the sled 40054 is part of the stapling attachment 40010 and not part of the staple cartridge 40050.

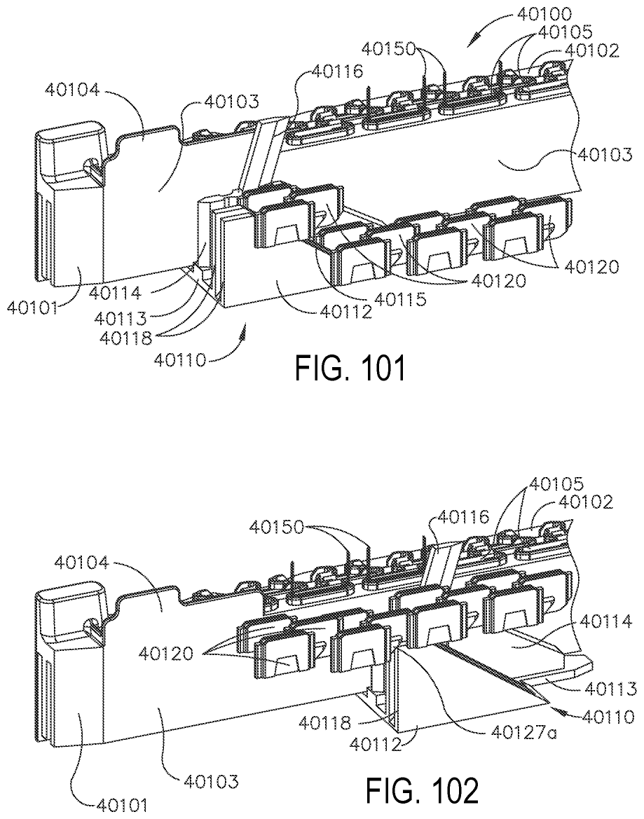

FIGS. 101-105 depict a staple cartridge 40100 for use with a surgical instrument. The staple cartridge 40100 comprises a cartridge body 40101, a sled 40110, a plurality of staple drivers 40120 arranged in longitudinal rows, and staples 40150 supported by the staple drivers 40120. The cartridge body 40101 includes a longitudinal slot 40103, a deck 40102, and a plurality of staple cavities 40105 define in the deck 40102. The cartridge body 40101 further comprises a proximal knife housing 40104 at the proximal end of the longitudinal slot 40103. Referring primarily to FIG. 101, three rows of staple cavities 40105 are positioned on a first side of the longitudinal slot 40103 and three rows of staple cavities 40105 are positioned on a second side of the longitudinal slot 40103 opposite the first side. Other arrangements of staple cavities and staples are contemplated. In use, the staple cartridge 40100 is positioned on a first side of patient tissue that is to be stapled and an anvil, such as the anvil 40034, for example, is positioned on a second side of the patient tissue. The anvil 40034 is moved toward the staple cartridge 40100 to compress and clamp the tissue against the deck 40055, however, in at least one other embodiment, the staple cartridge 40100 is moved toward the anvil 40034 to compress and clamp tissue against the deck 40055. After the patient tissue has been clamped, or captured between, the jaws, the staple firing stroke can be performed, as discussed further below.

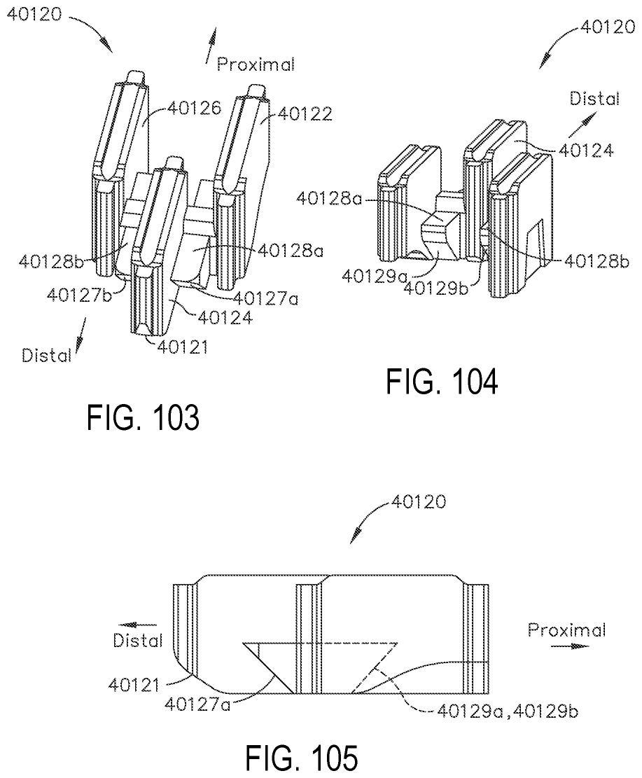

During the staple firing stroke, the sled 40110 is pushed distally by the firing driver 40040 to engage the staple drivers 40120 and eject the staples 40150 supported on the staple drivers 40120 into the captured patient tissue. The staple drivers 40120 are movably positioned in the staple cavities 40105 and are movable between a first, or unfired, position and a second, or fired, position by the sled 40110 to eject the staples 40150 from the staple cavities 40105. FIG. 103 illustrates that the staple drivers 40120 are triple staple drivers configured to simultaneously eject three staples; however, other staple drivers are contemplated which are configured to eject one, two, or more than three staples. In at least one instance, the drivers 40120 are retained in the cartridge body 40101 by a retainer, or pan, which extends at least partially around the bottom of the cartridge body 40101 and includes resilient members configured to grip the cartridge body 40101 and hold the retainer to the cartridge body 40101. The drivers 40120 are movable between their unfired positions and their fired positions by the sled 40110 to eject the staples from the cartridge body 40101, as discussed in greater detail below.

Returning to FIG. 101, the sled 40110 comprises a base 40113, a plurality of rails 40112 extending upwardly from the base 40113, and a central portion 40114 extending upwardly from the base 40113. The central portion 40114 comprises a knife 40116 extending therefrom. Further, each of the rails 40112 comprises a distal ramp portion 40115 thereon. The sled 40110 is movable distally from a proximal unfired position to a distal fired position during a staple firing stroke and movable proximally from the distal fired position to the proximal unfired position during a retraction stroke by a firing member of the surgical instrument, such as the firing driver 40040 (see FIG. 100). The central portion 40114 and the knife 40116 of the sled 40110 are configured to move within the longitudinal slot 40103 of the cartridge body 40101 during the staple firing stroke and the retraction stroke. At least a portion of the knife 40116 is stored in the proximal knife housing 40104 of the cartridge body 40101 when the sled 40110 is in the proximal unfired position. The knife 40116 comprises a distally-presented cutting edge that is exposed when the sled 40110 is moved distally at the beginning of the staple firing stroke. Further, as discussed in greater detail below, the distal ramps 40115 of the sled 40110 engage each of the staple drivers 40120 and move the staple drivers 40120 from their unfired positions to their fired positions during the staple firing stroke.

Referring primarily to FIGS. 103-105, each staple driver 40120 comprises a plurality of staple supports comprising an inner staple support 40122, a central staple support 40124, and an outer staple support 40126. The inner staple support 40122 is positioned closest to the longitudinal slot 40103 of the cartridge body 40101 and the outer staple support 40126 is positioned farthest away from the longitudinal slot 40103 of the cartridge body 40101. Each of the staple supports 40122, 40124, 40126 are configured to support a staple 40150 thereon. The inner staple support 40122 is connected to the central staple support 40124 by an inner lateral bridge 40128a and the outer staple support 40126 is connected to the central staple support 40124 by an outer lateral bridge 40128b. The inner lateral bridge 40128a comprises an inner proximal camming surface 40129a and the outer lateral bridge 40128b comprises an outer proximal camming surface 40129b which are positioned on opposite sides of the central staple support 40124. In use, the distal ramps 40115 of the sled 40110 on one side of the longitudinal slot 40103 are configured to engage the inner proximal camming surface 40129a and the outer proximal camming surface 40129b of each driver 40120 to eject the staples 40150 positioned on the staple supports 40122, 40124, 40126 from the cartridge body 40101 during the staple firing stroke. Similarly, the distal ramps 40115 on the other side of the sled 40110 engage the drivers 40120 on the other side of the longitudinal slot 40103 to eject the staples positioned thereon.

Further to the above, once all of the staple drivers 40120 have been moved from their unfired positions to their fired positions, the sled 40110 is retracted from its distal fired position toward a proximal position. In various instances, the staple drivers 40120 are sized and configured such that they fit closely within their respective staple cavities such that, once such staple drivers 40120 are moved to their fired positions, they remain in their fired positions. In at least one instance, the perimeters of the staple supports 40122, 40124, 40126 have a line-to-line fit with the perimeters of the staple cavities In such instances, the sled 40110 may easily pass under the staple drivers 40120 as the sled 40110 is retracted proximally by the firing driver 40040. In certain instances, however, one or more of the staple drivers 40120 may fall into or toward their unfired position and into the path of the sled 40110 as the sled 40110 is being retracted. FIG. 102 illustrates the sled 40110 during a retraction stroke where one of the triple staple drivers 40120 has fallen slightly from its fired position and into the path of the rails 40112 of the sled 40110. Specifically, the inner and outer lateral bridges 40128a, 40128b of the staple driver 40120 are aligned with the rails 40112 of the sled 40110, as discussed above, and therefore the inner and outer lateral bridges 40128a, 40128b are positioned in the return path of the rails 40112 of the sled 40112 in FIG. 102.

Further to the above, referring to FIG. 103, the inner and outer lateral bridges 40128a, 40128b of the staple drivers 40120 further comprise inner and outer distal camming surfaces 40127a, 40127b thereon. As such, when the sled 40110 is retracted, proximal vertical faces 40118 of the sled rails 40112 engage the inner and outer distal camming surfaces 40127a, 40127b of the fallen driver 40120 to move the fallen driver 40120 toward its fired position to permit the sled 40110 to pass thereunder and be retracted. In other words, once the fallen driver 40120 is moved back into its fired position, the sled 40110 is able to pass underneath the fallen driver 40120 and move to its proximal or fully retracted position. In certain instances, one or more staple drivers 40120 may move completely into their unfired positions such that, when the sled 40110 is retracted, the interaction between the proximal vertical faces 40118 of the sled rails 40112 and the inner and outer distal camming surfaces 40127a, 40127b of the fallen driver 40120 may not be enough to lift the staple driver 40120 from its unfired position. As such, it may be advantageous to include a proximally facing ramp on the sled rails 40112 to aid in lifting the driver 40120 from its unfired position toward its fired position, as discussed in greater detail below.

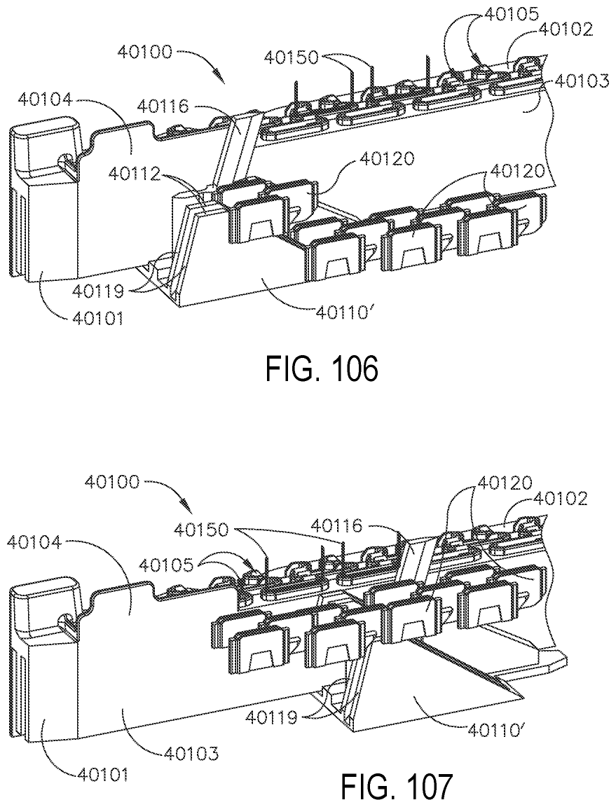

FIGS. 106 and 107 illustrate a sled 40110′ for use with the staple cartridge 40100. The sled 40110′ comprises rails 40112 which each includes a proximal ramp 40119 facing proximally. The proximal ramps 40119 are configured to engage the inner and outer distal camming surfaces 40127a, 40127b of a dropped or fallen staple driver 40120 when the sled 40110′ is retracted. Notably, the proximal ramps 40119 extend from the top of the rails 40112 all the way down to the bottom of the sled 40110′. As such, the proximal ramps 40119 are configured to engage a staple driver 40120 in any position—partially fallen or completely fallen—and lift the staple driver 40120 upwardly during the retraction stroke to make clearance for the sled 40110′. In various embodiments, however, the angle on the ramps 40119 can be steep. Such embodiments may be common when there are space constraints within the surgical instrument. As such, in various embodiments, a sled can comprise features that first lift a fallen staple driver from its unfired position to an intermediate position using a first portion of the sled and then lift the fallen staple driver from the intermediate position into its fired position using a second portion of the sled, as discussed in greater detail below.

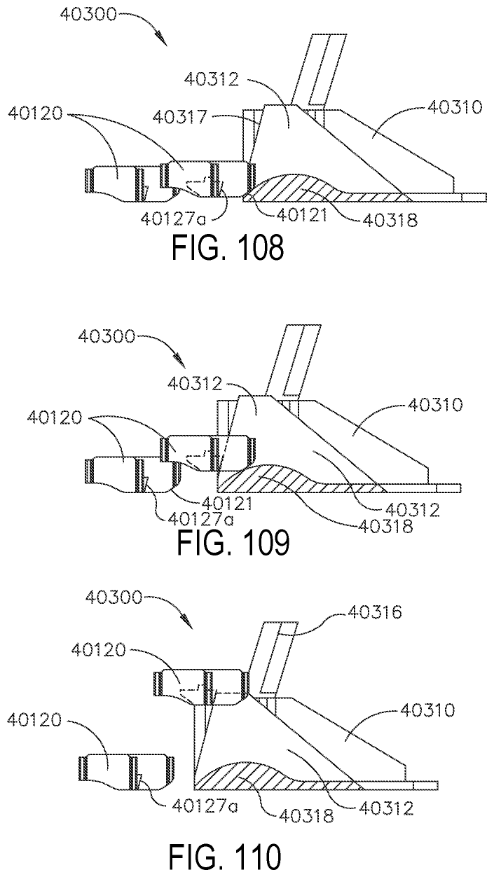

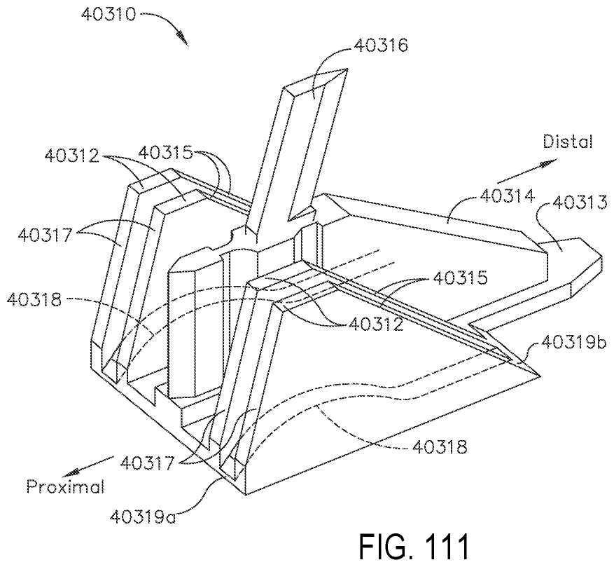

FIGS. 108-111 illustrate a staple cartridge 40300 for use with a surgical stapling instrument, such as those described herein. The staple cartridge 40300 is similar to the staple cartridges 40100 in many respects. For instance, the staple cartridge 40300 comprises the cartridge body 40101, the deck 40102, the longitudinal slot 40103, the staple cavities 40105, the staple drivers 40120, and the staples 40150, discussed above. Further, the staple cartridge 40300 comprises a sled 40310 that is movable distally during a staple firing stroke to drive the staple drivers 40120 upwardly and eject the staples 40150 stored in the staple cartridge 40300. The sled 40310 comprises a base 40313, a plurality of rails 40312 extending upwardly from the base 40313, and a central portion 40314 also extending upwardly from the base 40313 (see FIG. 111). The central portion 40314 comprises a knife 40316 extending therefrom and is configured to translate within a longitudinal slot defined in the staple cartridge 40300. Each of the rails 40312 comprises a distal ramp portion 40315 facing distally that is configured to engage the staple drivers 40120.

Further to the above, the region of the base 40313 of the sled 40310 in between the rails 40312 at the distal end 40319b of the base 40313 is configured to provide an initial lift to one or more of the staple drivers 40120 during the firing stroke of the sled 40310. Specifically, the distal end 40319b of the base 40313 in between the rails 40312 is configured to contact the central staple support 40124 of the staple driver 40120 prior to the distal ramps 40315 of the sled 40310 engaging an inner proximal camming surface 40129a and an outer proximal camming surface 40129b of the staple driver 40120. As such, the base 40313 of the sled 40310 is configured to initially lift the staple driver 40120 from its unfired position to an intermediate position and then the distal ramps 40315 of the sled 40310 are configured to engage the staple driver 40120 to move the staple driver 40120 from the intermediate position to the fired position.

In use, further to the above, the sled 40310 is movable distally from a proximal position to a distal position during a staple firing stroke and movable proximally from the distal position to the proximal position during a retraction stroke by a firing driver of a surgical instrument, such as the firing driver 40040 (see FIG. 100). During the staple firing stroke, as described above, the distal ramps 40315 of the sled 40310 are configured to engage the staple drivers 40120 to move the staple drivers 40120 from their unfired positions to their fired positions. Once the firing stroke is complete, further to the above, one or more of the staple drivers 40120 may fall from their fired positions toward and/or into their unfired positions and, thus, into the path of the rails 40312 of the sled 40310. As discussed in greater detail below, the sled 40310 is configured to move the fallen staple drivers 40120 into their fired positions during the retraction stroke to permit the sled 40310 to be retracted.

Further to the above, referring again to FIG. 111, each lateral side of the sled 40310 comprises two rails 40312. In addition to a distal ramp 40315, each rail 40312 further comprises a proximal ramp portion 40317 facing proximally. The base 40313 comprises a proximal end 40319a and a distal end 40319b, with the distal end 40319b located in the region of the rails 40312. The base 40313 further comprises a driver lift cam 40318 positioned intermediate the rails 40312 on each side of the central portion 40314 of the sled 40310. Referring primarily to FIG. 111, each driver lift cam 40318 comprises an arcuate protrusion extending upwardly from the base 40313. The driver lift cam 40318 tapers downwardly toward the proximal end 40319a of the base 40313 such that the thickness of the base 40313 at the proximal end 40319a is reduced or recessed, i.e., less than the general thickness of the base 40313. The drive lift cams 40318 and the proximally facing ramps 40317 of the sled 40310 are configured to engage and lift one or more fallen staple drivers 40120 toward their fired positions during a retraction stroke of the sled 40310, as discussed in greater detail below.

FIG. 108 illustrates two of the drivers 40120 which have fallen from their fired positions with the sled 40310 positioned distal to the drivers 40120 after the firing stroke has been completed, or at least partially completed. As the sled 40310 is retracted proximally from its position in FIG. 108, a driver lift cam 40318 of the sled 40310 engages a distal ramp 40121 (see FIGS. 103 and 105) of the central staple support 40124 of the staple driver 40120 to initially move the staple driver 40120 from a fallen position toward its fired position as shown in FIG. 108. Further proximal retraction of the sled 40130 results in the driver lift cam 40318 continuing to engage the distal ramp 40121 to move the staple driver 40120 into an intermediate position illustrated in FIG. 109. In various instances, the driver lift cam 40318 is configured to move the staple driver 40120 a minimum distance toward its fired position. In various instances, the minimum distance is between 0.033 to 0.045 inches (i.e., 0.083 to 0.114 cm), for example, or, in at least one embodiment, at least half of the overall height of the driver 40120.

Further to the above, when the staple driver 40120 is in the intermediate position, the proximal ramps 40317 of the sled 40130 abut the inner and outer distal camming surfaces 40127a, 40127b (see FIG. 103) of the staple driver 40120. As the sled 40130 is retracted further proximally from its position in FIG. 109 to its position in FIG. 110, the proximal ramps 40317 continue to engage the inner and outer distal camming surfaces 40127a, 40127b of the staple driver 40120 and move the staple driver 40120 from its intermediate position in FIG. 109 into its fired position (FIG. 110). Once the fallen staple drivers 40120 is moved into its fired position, the sled 40310 is permitted to pass by the staple drivers 40120. This same process is repeated for any of the staple drivers 40120 that may have moved from their fired positions into the retraction path of the sled 40130 as the sled 40130 is retracted to its proximal position.

Further to the above, the angle of the inner and outer distal camming surfaces 40127a, 40127b compliment the angle of the proximal ramps 40317. In at least one embodiment, the proximal ramps 40317 of the rails 40312 comprise a slope that matches the slope of the camming surfaces 40127a, 40127b. In various embodiments, the proximal ramps 40317 of the rails 40312 comprise a more gradual slope than the proximal ramps 40317 depicted in FIGS. 108-111. In at least one embodiment, the angles of the inner and outer distal camming surfaces 40127a, 40127b of the staple driver 40120 correspond to the gradual slope of the proximal ramps 40317. In certain instances, it may be advantageous to have as gradual of a proximal sloped ramp on the sled as possible without unnecessarily lengthening the sled 40310 in order to provide better mechanical leverage when lifting a fallen staple driver. In certain instances, as discussed in greater detail below, it is advantageous for the distal ramps 40315 to have a first angle and the proximal ramps 40317 to have a second angle that is different from the first angle.

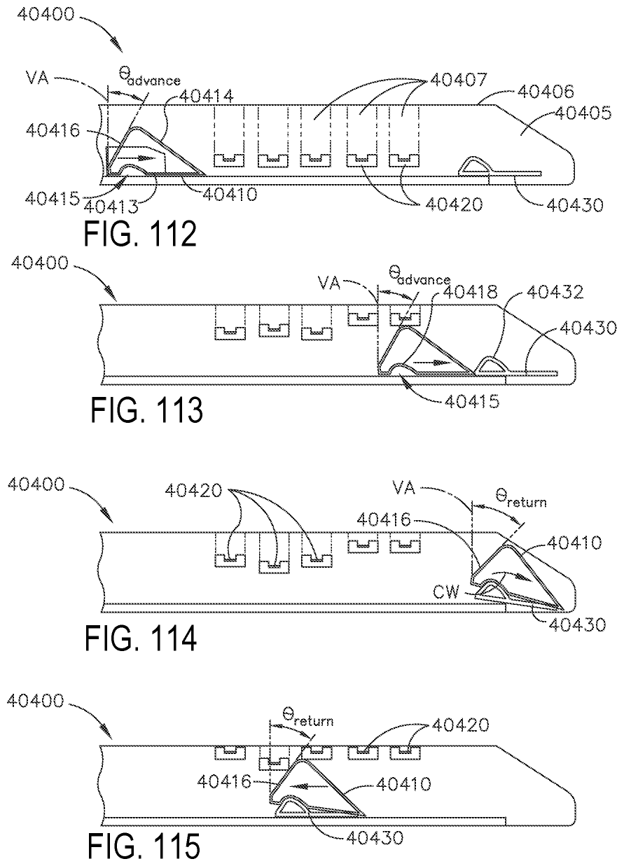

FIGS. 112-116 illustrate a staple cartridge 40400 for use with a surgical stapling instrument such as those disclosed herein. The staple cartridge 40400 comprises a cartridge body 40405, a sled 40410, a plurality of staple drivers 40420 arranged in longitudinal rows, staples (not shown in FIGS. 112-116) supported by the staple drivers 40420, and a sled lift cam 40430. The cartridge body 40405 includes a longitudinal slot defining a longitudinal axis, a proximal end, a distal end, and a deck 40406 extending intermediate the proximal end and the distal end. The cartridge body 40405 further comprises a plurality of staple cavities 40407 defined in the cartridge body 40405. The staple cavities 40407 define a plurality of staple cavity openings in the deck 40406 of the cartridge body 40405. In at least one instance, three rows of staple cavities 40407 are positioned on a first side of the longitudinal slot and three rows of staple cavities 40407 are positioned on a second side of the longitudinal slot. Other arrangements of staple cavities and staples are contemplated. In use, the staple cartridge 40400 is positioned on a first side of the tissue to be stapled and the anvil is positioned on a second side of the tissue to be stapled. The anvil is moved toward the staple cartridge to compress and clamp the tissue against the deck 40406, however, in at least one instance the staple cartridge 40400 is moved toward the anvil to compress and clamp tissue against the deck 40406. Thereafter, staples removably stored in the cartridge body 40405 can be deployed into the tissue.

Further to the above, the staple drivers 40420 may be similar or identical to the staple drivers 40120 discussed herein. However, other staple driver arrangements are contemplated. The staple drivers 40420 are movably positioned in the staple cavities 40407 such that their movement is constrained to vertical movement by the walls of the staple cavities 40407.

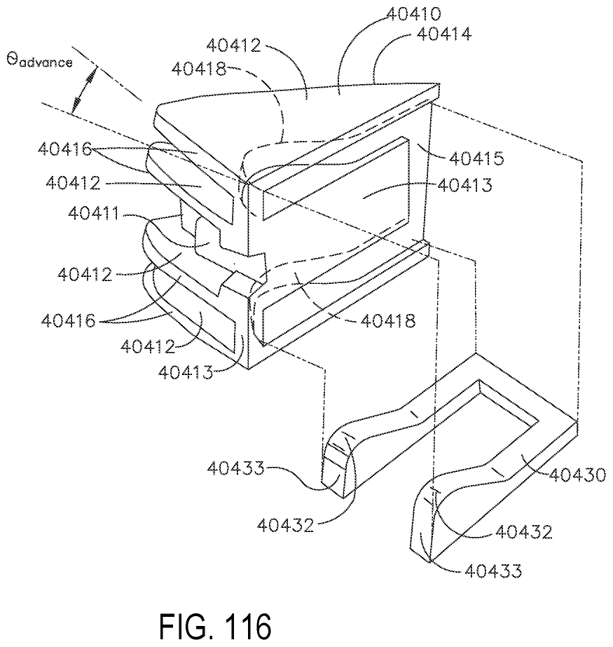

Referring primarily to FIG. 116, the sled 40410 comprises a base 40413, a central portion 40411, and a plurality of rails 40412 extending from the base 40413. As discussed herein, the central portion 40411 is configured to travel within a longitudinal slot of the staple cartridge body 40405. In at least one instance, the central portion 40411 comprises a knife, such as the knife 40316 (FIG. 111), for example. Further, FIG. 116 illustrates that there are two rails 40412 on each side of the central portion 40411. Each of the rails 40412 comprises a distal ramp 40414 facing distally and a proximal ramp 40416 facing proximally. The base 40413 of the sled 40410 further comprises a driver lift cam 40418 at its proximal end intermediate the rails 40412 on both sides of the central portion 40411. The sled 40410 further comprises a bottom cutout region 40415 defined in the base 40413 of the sled 40410. The cutout region 40415 extends proximally in between the rails 40412 on each side of the central portion 40411 of the sled 40410 and at least partially into the driver lift cam 40418. The driver lift cam 40418 may be similar to or identical to the driver lift cam 40318 (FIG. 111) and operate in a similar or identical manner, for example. The cutout region 40415 is defined in the base 40413 and the driver lift cam 40418 to provide room for the sled lift cam 40430, as discussed in greater detail below.

Further to the above, the sled 40410 is movable from a proximal unfired position (FIG. 112) to a distal fired position (FIG. 114) during a firing stroke by a firing driver of the surgical instrument such as those discussed herein. Also further to the above, the sled 40410 is movable from the distal fired position (FIG. 114) to the proximal unfired position (FIG. 112) during a retraction stroke by the firing driver after at least a portion of the firing stroke is completed. In use, the distal ramps 40414 of the sled 40410 are configured to engage and move the staple drivers 40420 from an unfired position (FIG. 112) to a fired position during the firing stroke. After the firing stroke is completed, one or more of the staple drivers 40420 may move, or fall, from their fired positions toward their unfired positions, as depicted in FIG. 113. Depending on various circumstances, the staple drivers 40420 may move various amounts toward their unfired position after the firing stroke is completed depending on, among other things, the fit of the driver 40420 within its corresponding staple cavity 40407. As such, one or more drivers 40420 may only drop slightly whereas other drivers 40420 may drop entirely into their unfired positions.

During the firing stroke, as discussed above, the sled 40410 is moved distally toward the distal end of the staple cartridge 40400. Notably, the sled lift cam 40430 does not travel distally with the sled 40410. Also notably, the proximal ramps 40416 of the sled 40410 are oriented at an angle θ_{advance} relative to vertical axis VA. As the sled 40410 approaches the distal fired position, the cutout region 40415 of the sled 40410 at least partially receives the sled lift cam 40430 therein as illustrated in FIG. 114. Specifically, the distal end of the advancing sled 40410 rides up and over proximal ramps 40433 of the sled lift cam 40430 until the sled lift cam 40430 is received in the cutout region 40415 of the sled 40410. In such instances, a proximal protrusion 40432 of the sled lift cam 40430 is received in the cutout region 40415 of the sled 40410 beneath the driver lift cam 40418. Notably, at least a portion of the sled lift cam 40430 is vertically larger than cutout region 40415 of the sled 40410 in which it is received. As such, the orientation of the sled 40410 is changed, i.e. the sled 40410 is rotated in direction CW, when the sled 40410 receives the sled lift cam 40430.

During the retraction stroke of the sled 40410, the sled lift cam 40430 remains engaged with the sled 40410 and the proximal ramps 40416 of the sled are oriented at angle θᵣₑₜᵤᵣₙ relative to the vertical axis VA. Further, referring primarily to FIGS. 113 and 114, angle θᵣₑₜᵤᵣₙ is greater than angle θ_{advance}. As such, the proximal ramps 40416 of the sled 40410 have a more gradual slope relative to the vertical axis VA during the retraction stroke as compared to during the firing stroke. The proximal ramps 40416 of the sled 40410 are configured to interact with fallen staple drivers 40420 at angle θᵣₑₜᵤᵣₙ as comparted angle θ_{advance} which is steeper than θᵣₑₜᵤᵣₙ. The more gradual slope of the proximal ramps 40416 during the retraction stroke may be advantageous in lifting dropped staple drivers 40420, for example. Moreover, utilizing the sled lift cam 40430 to change the angle of the proximal ramps 40416 during the retraction stroke creates a compact assembly.

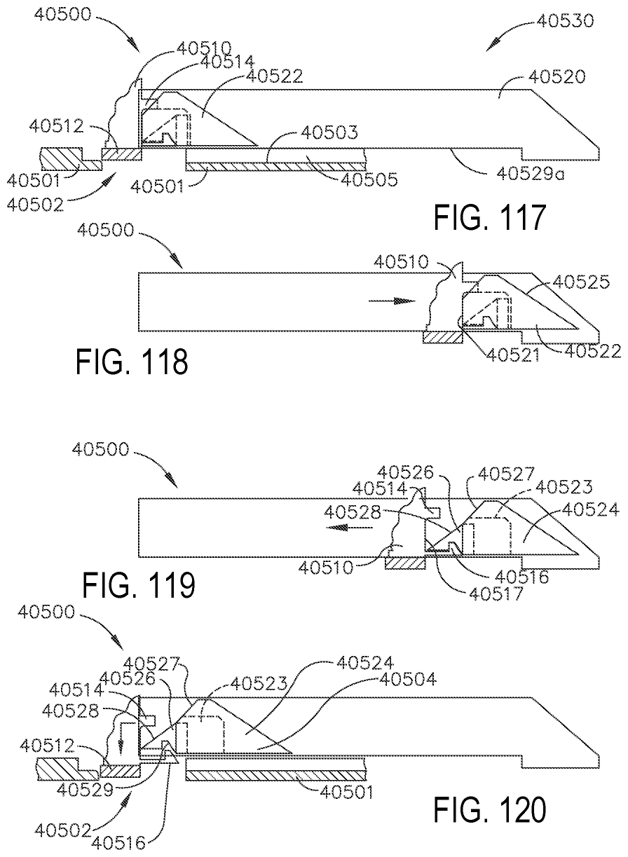

FIGS. 117-120 illustrate a surgical instrument 40500 comprising an end effector 40530 and a firing driver 40510 movable within the end effector 40530. The end effector 40530 comprises a first jaw 40501 and a second jaw movable relative to the first jaw 40501 to capture tissue in between the first jaw 40501 and the second jaw. In at least one embodiment, the first jaw 40501 is movable relative to the second jaw. The first jaw 40501 is configured to receive a staple cartridge 40520 therein as illustrated in FIG. 117. The staple cartridge 40520 comprises staple drivers, such as those discussed herein, and a sled 40522 configured to move distally to engage the staple drivers and eject staples from the staple cartridge 40520 during a staple firing stroke. In at least one aspect, the staple cartridge 40520 is configured to be inserted into the first jaw 40501 with the sled 40522 in a proximal unfired position and the firing driver 40510 positioned proximal to the sled 40522. The firing driver 40510 is then distally advanced toward the sled 40522 after the staple cartridge 40520 is inserted into the first jaw 40501 and defeats a firing lockout, as discussed in greater detail below.

When the staple cartridge 40520 is seated in the first jaw 40501, as illustrated in FIG. 117, a longitudinal cavity, or gap, 40505 exists between the first jaw 40501 and the staple cartridge 40520. The longitudinal cavity 40505 is defined by a bottom surface 40529a of the staple cartridge 40520 and a top surface 40503 of the first jaw 40501. The staple cartridge 40520 is attached or affixed to the first jaw 40501 such that the bottom surface 40529a of the staple cartridge 40520 is vertically spaced apart from the top surface 40503 of the first jaw 40501 to create the longitudinal cavity 40505. However, other embodiments are envisioned in which the longitudinal cavity 40505 is defined entirely within the first jaw 40501, for example.

Further to the above, the firing driver 40510 comprises a lower laterally extending foot 40512 configured to move within the longitudinal cavity 40505 during a staple firing stroke and a retraction stroke. The firing driver 40510 further comprises a flexible tab 40516 and a nose 40514 extending distally from the firing driver 40510. The firing driver 40510 is biased toward the first jaw 40501 by a biasing member, such as a spring, for example, positioned against a top surface of the firing driver 40510.

When the staple cartridge 40520 is seated in the first jaw 40501 and sled 40522 of the staple cartridge 40520 is in its proximal unfired position, as illustrated in FIG. 117, and the firing driver 40510 is advanced distally to perform a staple firing stroke, the nose 40514 of the firing driver 40510 rests on a ledge 40523 of the sled 40522 to prevent the foot 40512 of the firing driver 40510 from entering a lockout opening 40502 in the first jaw 40501. If, however, the firing driver 40510 is advanced distally without the staple cartridge 40520 being seated in the first jaw 40501, or advanced distally when the staple cartridge 40520 is seated in the first jaw 40501 but the sled 40522 is not in its proximal unfired position at the outset of the staple firing stroke, the nose 40514 of the firing driver 40510 will not be supported by the ledge 40523 of the sled 40522 and the firing driver 40510 will be biased into the lockout opening 40502 by the biasing member and is, as a result, prevented from being advanced through the staple firing stroke. This arrangement provides a missing cartridge lockout as well as a spent cartridge lockout. Once the missing cartridge/spent cartridge lockout has been overcome by seating an unspent staple cartridge 40520 in the first jaw 40501 and the firing driver 40510 is moved distally to perform the staple firing stroke, the flexible tab 40516 of the firing driver 40510 is received in a recess 40529 of the sled 40522 to operably couple the firing driver 40510 and the sled 40522, as discussed in greater detail below.

The sled 40522 comprises a first portion 40524 and a second portion 40526 housed within the first portion 40524. The second portion 40526 is slidably attached to the first portion 40524 such that the second portion 40526 is movable relative to the first portion 40524. In one aspect, the second portion 40526 comprises a protrusion that extends into a longitudinal slot of the first portion 40524 to slidably couple the first portion 40524 and the second portion 40526. Other attachment methods are contemplated for slidably attaching the first portion 40524 and the second portion 40526 together. The first portion 40524 comprises at least one distal ramp 40525 facing distally that is configured to engage the staple drivers of the staple cartridge 40520 during the firing stroke. The first portion 40524 further comprises a first proximal ramp 40527 facing proximally. The second portion 40526 comprises the recess 40529 discussed above and a second proximal ramp 40528. The sled 40522 further comprises a knife, or tissue cutting member, such as a knife 40316, for example. That said, other embodiments are envisioned in which the sled 40522 does not comprise a knife or tissue cutting member.

As discussed above, the firing driver 40510 is advanced distally to engage the sled 40522. Specifically, a distal end 40517 of the firing driver 40510 abuts a proximal end 40521 of the second portion 40526 to advance the sled 40522 distally as the firing driver 40510 is advanced distally by a firing drive. When the firing driver 40510 is advanced from the position in FIG. 117 to the position in FIG. 118 during a staple firing stroke, the distal ramp 40525 of the sled 40522 engages the staple drivers to eject the staples from the staple cartridge 40520. After the firing stroke has been completed, or at least partially completed, the firing driver 40510 is retracted in the proximal direction from the position in FIG. 118 to the position in FIG. 119 during a retraction stroke. When the firing driver 40510 moves from the position in FIG. 118 to the position in FIG. 119, the second portion 40526 of the sled 40522 is pulled proximally relative to the first portion 40524 of the sled 40522 to transition the sled 40522 from a collapsed configuration (FIG. 118) to an expanded configuration (FIG. 119). Specifically, the flex tab 40516 of the firing driver 40510 is engaged with the recess 40529 of the second portion 40526 of the sled 40522 such that when the firing driver 40510 is pulled proximally, the second portion 40526 is pulled proximally relative to the first portion 40524. Once the second portion 40526 is completely expanded relative to the first portion 40524, the first and second portions 40524, 40526 are pulled proximally together by the firing driver 40510 to complete the retraction stroke.

Further to the above, the second proximal ramp 40528 of the second portion 40526 is exposed when the sled 40522 is in its expanded configuration as illustrated in FIG. 119. The second proximal ramp 40528 is configured to work in conjunction with the first proximal ramp 40527 of the first portion 40524 to engage the staple drivers that have fallen down from their fired positions. Specifically, the proximal ramps 40527, 40528 are configured to cammingly engage and lift one or more of the staple drivers which may be in the way of the sled 40522 during the retraction stroke to permit the sled 40522 to complete the retraction stroke.

Further to the above, when the sled 40522 is transitioned from the collapsed configuration (FIG. 118) to the expanded configuration (FIG. 119), the distal nose portion 40514 of the firing driver 40510 no longer rests on the ledge 40523 of the sled 40522. As such, when the sled 40522 is retracted to the position illustrated in FIG. 120, the foot 40512 of the firing driver 40510 is biased into the lockout opening 40502 by the biasing member. As such, the firing driver 40510 is prevented from advancing the sled 40522 again if the spent staple cartridge 40520 remains positioned in the first jaw 40501. Further, when the sled 40522 is in the proximal unfired position depicted in FIG. 120, the second portion 40526 of the sled 40522 is visible to a user of the surgical instrument 40500 through the lockout opening 40502 to indicate to the user that the staple cartridge 40520 has been fired, or at least partially fired. After the retraction stroke has been completed, the now-spent 40520 staple cartridge can be removed from the first jaw 40501. After the staple cartridge 40520 is removed from the first jaw 40501, the locked out firing driver 40510 is prevented from being advanced distally through another staple firing stroke unless an unfired staple cartridge 40520—with the sled 40522 in the proximal unfired position—is installed into the first jaw 40501. In various instances, an unfired staple cartridge 40520 can be inserted into the first jaw 40501 at an angle such that the ledge 40523 of the sled 40522 engages the distal nose 40514 of the firing driver 40510 to lift the firing driver 40510 out of the lockout opening 40502 which, as discussed above, defeats the missing cartridge/firing cartridge lockout and permit the firing driver 40510 to be advanced distally through another staple firing stroke.

During the retraction stroke of the firing driver 40510, referring again to FIG. 120, the first portion 40524 of the sled 40522 will not retract proximally all the way to its original proximal unfired position (i.e., the position in FIG. 117 when the staple cartridge 40520 was seated in the first jaw 40501). Specifically, the sled 40522 is retracted to a proximal returned position illustrated in FIG. 120 during the retraction stroke. The proximal returned position of the sled 40522 is distal to the proximal unfired position of the sled 40522. As such, if the spent staple cartridge 40520 with the sled 40522 in the position of FIG. 120 (i.e., the proximal returned position) is not removed from the end effector 40530, or is removed and re-inserted, the ledge 40523 of the sled 40522 will not be positioned proximal enough in the first jaw 40501 to defeat the lockout discussed above.

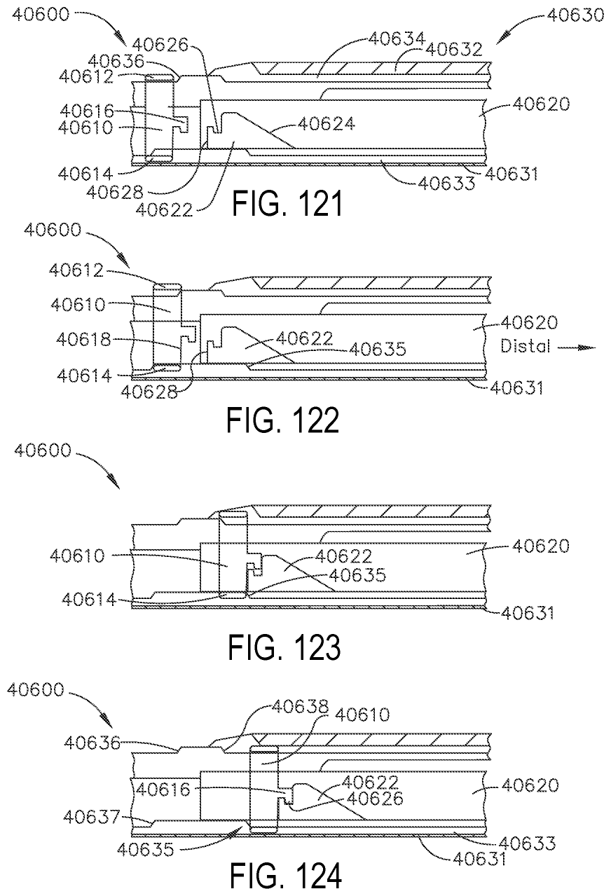

FIGS. 121-128 illustrate a surgical instrument 40600 comprising a firing driver 40610 and an end effector 40630 configured to receive a staple cartridge 40620. The firing driver 40610 is configured to move relative to the end effector 40630. The end effector 40630 comprises a first jaw 40631 and a second jaw 40632 movable relative to the first jaw 40631 to capture tissue in between the first jaw 40631 and the second jaw 40632. In at least one other embodiment, the first jaw 40631 is movable relative to the second jaw 40632. The staple cartridge 40620 comprises a plurality of staple drivers, such as those described herein, and a sled 40622 movable distally by the firing driver 40610 to eject staples from the staple cartridge 40620 during a staple firing stroke. The sled 40622 comprises at least one distal ramp 40624 configured to cammingly engage the staple drivers to eject the staples. In at least one instance, the sled 40622 comprises a cutting member, such as a knife, for example, configured to incise patient tissue captured between the first jaw 40631 and the second jaw 40632. However, in at least one instance, the firing driver 40610 comprises the cutting member instead of the sled 40622. Further, FIG. 121 illustrates the first jaw 40631 and the second jaw 40632 in a closed configuration with the sled 40622 of the staple cartridge 40620 in an unfired position and the firing driver 40610 in a proximal position after the staple cartridge 40620 has been inserted into the first jaw 40631. The firing driver 40610 comprises a first lateral cam portion 40614 configured to engage the first jaw 40631 and a second lateral cam portion 40612 configured to engage the second jaw 40632 during the staple firing stroke, as discussed in greater detail below.

The first jaw 40631 of the end effector 40630 comprises a longitudinal cavity 40633 defined therein that is configured to receive the first cam 40614 of the firing driver 40610. Further, the second jaw 40632 comprises a longitudinal cavity 40634 defined therein that is configured to receive the second cam 40612 of the firing driver 40610. The first jaw 40631 comprises a proximal ramp portion 40637 and a distal ramp 40635 defined by the longitudinal cavity 40633 of the first jaw 40631. Similarly, the second jaw 40632 comprises a proximal ramp 40636 and a distal ramp 40638 defined by the longitudinal cavity 40634 of the second jaw 40632. At the outset of the staple firing stroke, the ramps 40635, 40637, 40636, 40638 of the first and second jaws 40631, 40632 move the firing driver 40610 up and down relative to the staple cartridge 40620 to engage the firing driver 40610 with the sled 40622 of the staple cartridge 40620, as discussed in greater detail below. Moreover, as discussed in greater detail below, the ramps 40635, 40637, 40636, 40638 of the first and second jaws 40631, 40632 move the firing driver 40610 up and down relative to the staple cartridge 40620 during the retraction stroke to disengage the firing driver 40610 from the sled 40622

Further to the above, the sled 40622 is presented in front of the firing driver 40610 when the staple cartridge 40620 is seated in the first jaw 40631, as depicted in FIG. 121. When the firing driver 40610 is initially advanced distally, i.e., from the position in FIG. 121 to the position in FIG. 122, the second cam 40612 engages the proximal ramp 40636 of the second jaw 40632 which lifts the firing driver 40610 upwardly away from the first jaw 40631. As the firing driver 40610 advances further distally from the position in FIG. 122 to the position in FIG. 123, a distal face 40618 of the firing driver 40610 engages a proximal face 40628 of the sled 40622 to advance the sled 40622. As the firing driver 40610 advances from the position in FIG. 123 to the position in FIG. 124, the first cam 40614 of the firing driver 40610 engages the distal ramp 40635 of the first jaw 40631 which drives the firing driver 40610 downward toward the first jaw 40631. Moreover, a distal hook portion 40616 of the firing driver 40610 engages a recess 40626 of the sled 40622 to operably couple the firing driver 40610 and the sled 40622 as the firing driver 40610 moves from the position in FIG. 123 to the position in FIG. 124. At such point, the firing driver 40610 is movable distally to advance the sled 40622 to the distal end of the staple cartridge 40620 during a staple firing stroke to eject the staples from the staple cartridge 40620.

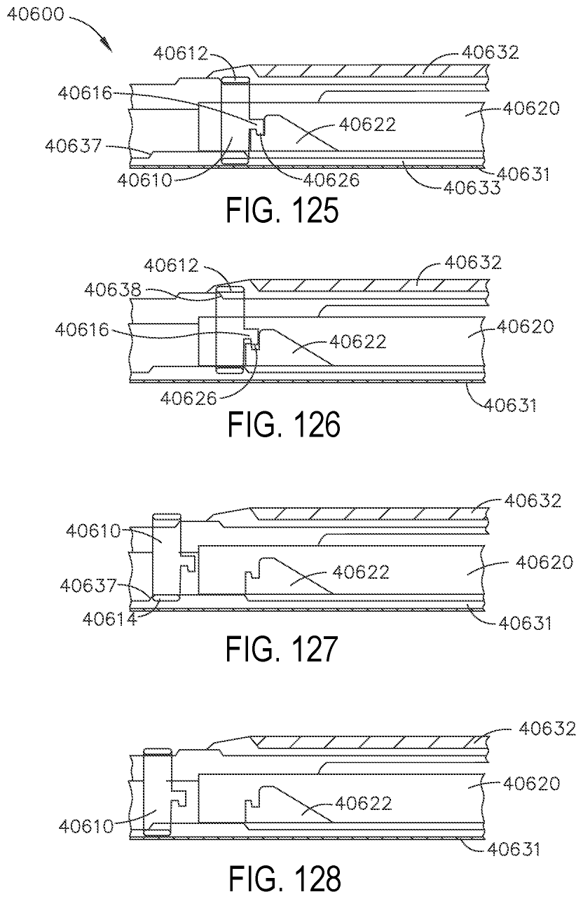

After the firing stroke is completed, further to the above, the firing driver 40610 is configured to retract the sled 40622 from its fired position at the distal end of the staple cartridge 40620 to the position in FIG. 125 during a retraction stroke. The sled 40622 is pulled proximally by the firing driver 40610 during the retraction stroke due to the engagement of the distal hook 40616 of the firing driver 40610 with the recess 40626 of the sled 40622. As the firing driver 40610 is retracted proximally from the position in FIG. 125 to the position in FIG. 126, a distal ramp 40638 of the second jaw 40632 drives the firing driver 40610 upward away from the first jaw 40631 and disengages the distal hook 40616 of the firing driver 40610 from the recess 40626 in the sled 40622. In at least one instance, the sled 40622 is constrained to only longitudinal movement within the staple cartridge 40620 such that, when the firing driver 40610 is driven upwardly, the sled 40622 remains stationary in the vertical direction. As the firing driver 40610 is retracted proximally from the position in FIG. 126 to the position in FIG. 127, the sled 40622 is not retracted and remains stationary. As the firing driver 40610 is retracted from the position in FIG. 127 to the position in FIG. 128, the first cam 40614 of the firing driver 40610 engages a proximal ramp 40637 of the first jaw 40631 which drives the firing driver 40610 downward away from the second jaw 40632. FIG. 128 illustrates the firing driver 40610 in the same proximal position as in FIG. 121. The spent staple cartridge 40620 can be removed from the end effector 40630 when the firing driver 40610 is in either of the positions depicted in FIG. 127 or FIG. 128, for example. If another staple cartridge 40620, i.e., an unspent staple cartridge 40620, is seated in the first jaw 40631—with the sled 40622 of the new staple cartridge 40620 in its unfired position—the firing driver 40610 can be advanced distally in the manner described above to complete another staple firing stroke. If, however, the firing driver 40610 is advanced distally without an unspent staple cartridge 40620 seated in the first jaw 40631—with the sled 40622 in its unfired position—the firing driver 40610 is stopped by a missing cartridge/spent cartridge lockout.

Further to the above, after the retraction stroke, the sled 40622 is in a more distal position in FIGS. 127 and 128 than the initial unfired position of the sled 40622 illustrated in FIG. 121. As such, if the spent staple cartridge 40620 with the sled 40622 in the position of FIG. 127 is not removed from the end effector 40630, or is removed and re-inserted, the firing driver 40610 will not be able to properly couple with the sled 40622.

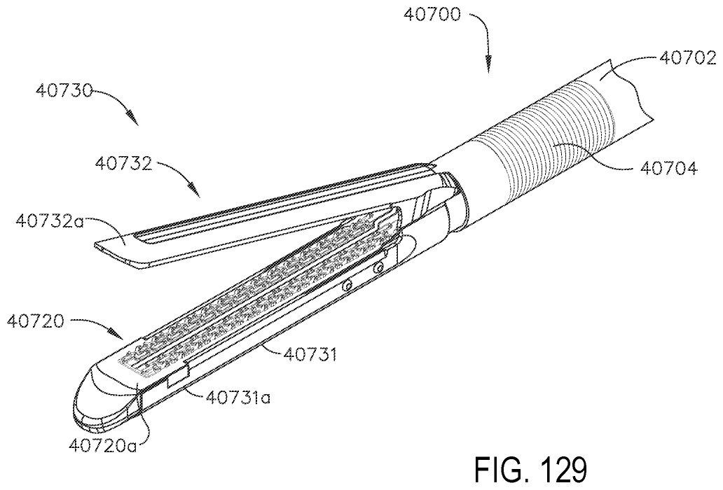

FIGS. 129-134 illustrate a portion of a surgical stapling instrument or surgical stapling system 40700. The surgical stapling instrument 40700 comprises a shaft 40702 and an end effector 40730 extending from the shaft 40702. In the illustrated example, the surgical stapling instrument comprises an articulation joint 40704 positioned intermediate the end effector 40030 and the shaft 40020. The articulation joint 40704 is configured to permit the end effector 40730 to rotate relative to the shaft 40702 about the articulation joint 40704. The end effector 40730 comprises a first jaw 40731 and a second jaw 40732 that is movable relative to the first jaw 40731 between an open position and a closed position to capture the tissue of a patient therebetween. In at least one other embodiment, the second jaw 40732 is movable relative to the first jaw 40731. Referring to FIG. 129, the first jaw 40731 comprises an elongate channel 40731a and the second jaw 40732 comprises an anvil 40732a. The elongate channel 40731a of the first jaw 40731 is configured to receive a replaceable staple cartridge 40720 therein that can be readily removed during a surgical procedure and replaced with a new staple cartridge; however, other embodiments are envisioned in which the first jaw 40731 comprises a staple cartridge that is not replaceable. The staple cartridge 40720 comprises a cartridge body 40720a, a sled 40722, a plurality of staple cavities 40750a arranged longitudinally in the cartridge body 40720a, wherein each staple cavity 40750a movably supports a corresponding staple driver 40750 therein, and wherein each staple driver 40750 operably supported one or more staples 40755 thereon.

Further to the above, the surgical stapling instrument 40700 further comprises a firing driver, sled driver or firing actuator 40710 that is movable relative to the end effector 40730 from a proximal position to a distal position during a firing stroke. The firing driver 40710 is configured to axially advance the sled 40722 within the staple cartridge 40720 from a proximal unfired position to a distal fired position during the firing stroke. The firing driver 40710 comprises a proximal portion 40712 and a distal head portion 40714 extending from the proximal portion 40712. In one arrangement, the proximal portion 40712 comprises a laminated beam configured to apply axial drive motions to the distal head portion 40714 yet capable of flexing to accommodate articulation of the end effector 40730. In other arrangements, the proximal portion 40712 may comprise a rod or rods, a beam or beams, an elongated actuator or actuators, etc. and be distally advanced from a starting position during a firing stroke and retracted proximally back to the starting position after the firing stroke by a manually-actuated drive arrangement, a motor-driven drive arrangement, a robotic drive system, a cable-driven system, etc.

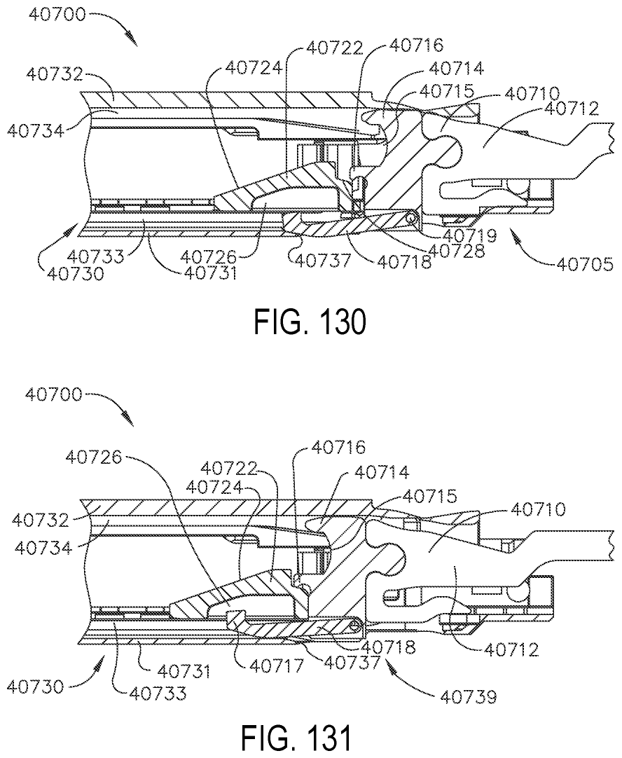

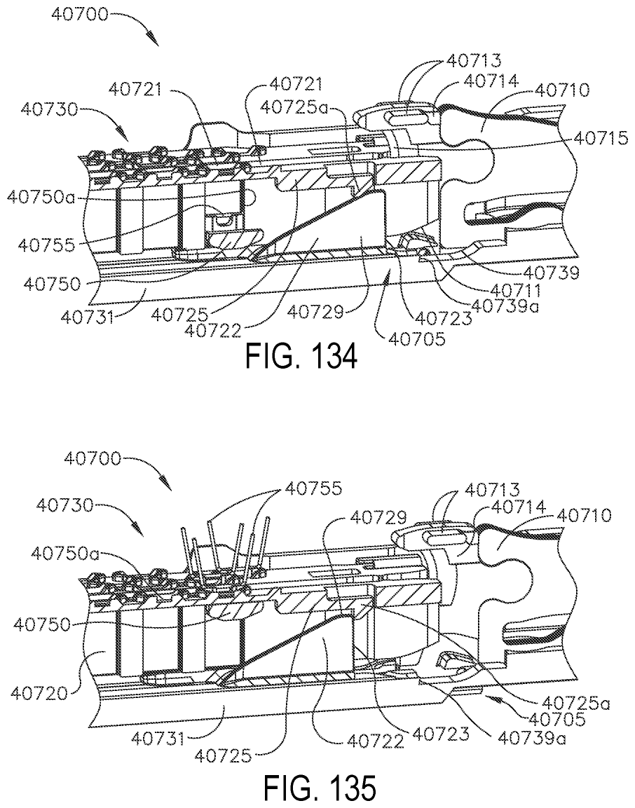

In the illustrated example, the distal head portion 40714 comprises a first lateral cam 40711 (see FIG. 134) that is configured to engage the first jaw 40731 and a second lateral cam 40713 that is configured to engage the second jaw 40732. As shown in FIGS. 130 and 131, the distal head portion 40714 of the firing driver 40710 comprises a knife 40715. However, at least one instance is envisioned where the sled 40722 comprises a knife or tissue-cutting feature instead of the distal head portion 40714. In the illustrated arrangement, the distal head portion 40714 comprises a distal nose 40716 and a latch arm 40718 that is pivotably connected to the distal head portion 40714 via a pin 40719. In at least one embodiment, the latch arm 40718 is biased downward toward the first jaw 40713 by a biasing member, such as a spring. Other attachment arrangements for pivotably connecting the arm 40718 to the distal head 40714 are contemplated. As can be seen in FIG. 131, a hook portion 40717 is formed at the distal end of the latch arm 40718. As will be discussed in further detail below, the hook portion 40717 is configured to hookingly engage a cavity 40726 in the sled 40722 to latch the sled 40722 to the distal head portion 40714 of the firing driver 40710.

The first jaw 40731 of the end effector 40630 comprises a longitudinal cavity 40733 configured to receive the first cam 40711 of the distal head portion 40714 of the firing driver 40710 during the firing stroke. In addition, the second jaw 40732 comprises a longitudinal cavity 40734 that is configured to receive the second cam 40713 of the distal head portion 40714 of the firing driver 40710 during the firing stroke. In at least one arrangement, the longitudinal cavity 40733 is further configured to slidably receive the latch arm 40718 and retain the latch arm 40718 in hooking engagement with the sled 40722 throughout the firing stroke and retraction stroke to ensure that the firing driver 40710 can be used to retract the sled at any time during the stapling process. As shown in FIG. 130, a proximal ramp portion 40737 is formed in the first jaw 40731 at the proximal end of the longitudinal cavity 40733 and, as will be discussed in further detail below, serves to move the latch arm 40718 into hooking engagement with the sled 40722 as the firing driver 40710 is distally driven from a starting position.

As can be seen in FIGS. 130, 131, 134, and 135, the sled 40722 comprises a distal ramp 40724 configured to cammingly engage the staple drivers 40750 of the staple cartridge 40720 to eject the staples 40755. FIG. 130 illustrates the first jaw 40731 and the second jaw 40732 in a closed configuration with the sled 40722 of the staple cartridge 40720 in an unfired position and the firing driver 40710 in a proximal position upon initial insertion of the staple cartridge 40720 into the first jaw 40731. The firing driver 40710 is configured to distally advance the sled 40722 through a firing stroke and proximally retract the sled 40722 through a retraction stroke, as discussed in greater detail below.

As was discussed above, when performing various stapling procedures using the surgical stapling instrument 40700, it is important that the staple cartridge 40720 that is installed in the end effector 40730 not only be compatible with the instrument 40700, but it must also be a fresh cartridge that has not been previously fired. If for example, the clinician unwittingly installed a previously fired cartridge into the end effector that was otherwise compatible with the end effector, the tissue may be severed but not stapled or otherwise fastened which could lead to catastrophic results. In the illustrated embodiment, the surgical stapling instrument 40700 is equipped with a firing driver lock, generally designated as 40705, which prevents the firing driver 40710 from being distally advanced through the staple cartridge 40720 during an attempted firing stroke. The firing driver lock 40705 comprises a lockout opening 40739 provided in the proximal end of the first jaw 40731. As will be further discussed below, the lockout opening 40739 is sized to receive therein a portion of the distal head portion 40714 of the firing driver 40710 unless a compatible unfired staple cartridge has been operably seated in the first jaw 40731. For example, in some configurations, the first cam 40711 on the distal head portion 40714 will be received in the lockout opening 40739 in the first jaw 40731 unless a compatible, unfired staple cartridge has been seated in the first jaw 40731. When the first cam 40711 of the distal head portion 40714 is received within the lockout opening 40739, the firing driver 40710 is prevent from moving distally should the clinician attempt to initiate the firing stroke. Stated another way, the first cam 40711 will contact a ledge 40739a (FIGS. 134 and 135) formed by the lockout opening 40739 in the first jaw 40731 and thereby prevent distal advancement of the firing driver 40710.

In use, the staple cartridge 40720 is inserted into the first jaw 40731 of the end effector 40730 with the sled 40722 in a proximal unfired position and the firing driver 40710 positioned proximal to the sled 47022. The firing driver 40710 is initially advanced toward the sled 40722 until the distal nose 40716 of the firing driver 40710 engages and rests on a ledge 40728 formed on the sled 40722. The distal nose 40716 of the firing driver 40710 can only rest on the ledge 40728 of the sled 40722 when the sled 40722 is in an unfired position. If the cartridge was previously fired, the sled of the cartridge would not be in the unfired position and could not engage the distal nose 40718 of the firing driver 40710. When the distal nose 40716 is received on the ledge 40728 on the sled 40722, the firing driver 40710 and, more particularly, the distal head portion 40714 of the firing driver 40710, is prevented from being biased into a lockout opening 40739 in the first jaw 40731. In one arrangement, the distal head portion 70714 of the firing driver 40710 is biased toward the lockout opening 40739 in the first jaw 40731 of the end effector 40730 by a biasing member such as a spring, for example. As such, if the firing driver 40710 is distally advanced without the staple cartridge 40720 present, or with the staple cartridge 40720 present and the sled 40722 is not in the proximal unfired position, the distal head portion 40714 of the firing driver 40710 will be biased into the lockout opening 40739 by the biasing member and, thus, the firing driver 40710 will be prevented from moving distally during an attempted firing stroke.

Further to the above, when the firing driver 40710 is advanced from the position in FIG. 130 to the position in FIG. 131, the latch arm 40718 of the firing driver 40710 engages the proximal ramp portion 40737 of the longitudinal cavity 40733 and is driven toward the sled 40722 such that the hook portion 40717 of the latch arm 40718 enters the cavity 40726 of the sled 40722. The firing driver 40710 is configured to abut and advance the sled 40722 from the position in FIG. 130 to a distal fired position during the firing stroke to eject the staples from the staple cartridge 40720. Further, the hook 40717 of the distal head portion 40714 remains positioned within the cavity 40726 during the firing stroke of the firing driver 40710 due to the interaction between the latch arm 40718 and the longitudinal cavity 40733.

After the firing stroke is completed, or when a partial firing stroke is performed, the firing driver 40710 is configured to retract the sled 40722 proximally to the released position illustrated in FIG. 134 during a retraction stroke. Specifically, the sled 40722 is pulled proximally by the firing driver 40710 during the retraction stroke due to the engagement of the distal hook 40717 of the arm 40718 of the firing driver 40610 with a proximal wall 40726a of the cavity 40726 of the sled 40722. As the firing driver 40710 retracts further proximally from the position in FIG. 133 to its original starting position, the latch arm 40718 pivots away from the sled 40722 to disengage the firing driver 40710 from the sled 40722. As discussed above, the arm 40718 of the firing driver 40710 is biased toward the first jaw 40731 by a biasing member such as a spring or the like. As such, when the firing driver 40710 is retracted, the arm 40718 is biased toward the proximal ramp portion 40737 of the first jaw 40731 and disengaged from the cavity 40726 of the sled 40722. In other words, the first jaw 40731 provides room for the arm 40718 to be biased out of the cavity 40726 of the sled 40722 when the firing driver 40710 is retracted back to its starting position. Further, the spent, or at least partially spent, staple cartridge 40720 can then be removed from the first jaw 40731.

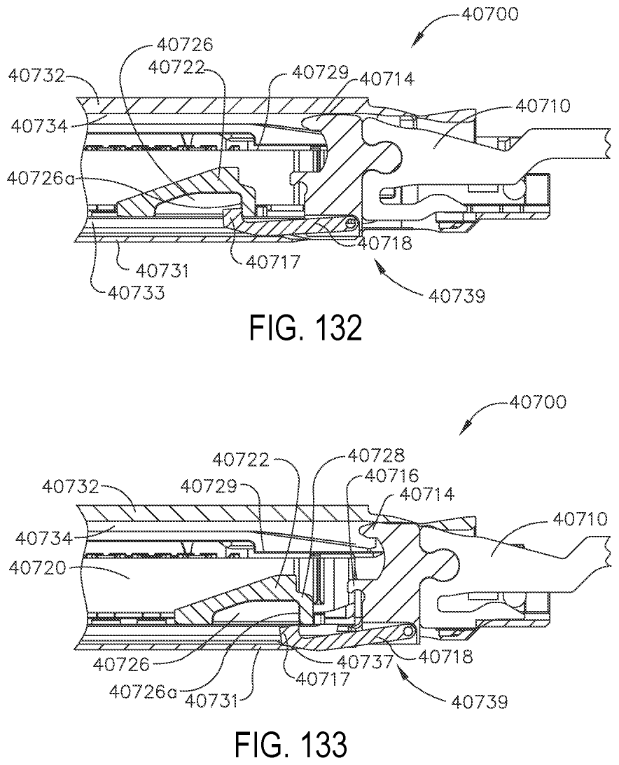

As discussed above, as the firing driver 40710 is retracted back to its starting position, the distal head portion 40714 of the firing driver 40710 disengages from the sled 40722 as depicted in FIGS. 132 and 133. As can be seen in FIG. F.5, when the firing driver 40710 is retracted into its original starting position, the sled 40722 has been proximally moved into the released position that is distal to its original proximal unfired position (i.e., the position of the sled 40722 in FIGS. 130 and 131). When the firing driver 40710 is retracted, the first cam 40711 of the firing driver 40710 is biased into the lockout opening 40739 as the distal nose 40716 no longer rests on the ledge 40728 of the sled 40722 to prevent the first cam 40711 from entering the lockout opening 40739. Further, when the distal head portion 40714 of the firing driver 40710 enters the lockout opening 40739, the first cam 40711 is viewable to a user of the surgical stapling instrument 40700 from the bottom of the elongate channel 40731a and therefore indicates to the user that the surgical attachment 40700 is locked out (i.e., the firing driver 40710 is prevented from performing a firing stroke). After the firing driver 40710 is retracted and the latch arm 40718 is disengaged from the sled 40722, the staple cartridge 40720 can be removed from the end effector 40730 and replaced with another staple cartridge 40720 with another sled 40722 in the proximal unfired position. The new unspent staple cartridge can be inserted into the end effector 40730 such that the sled 40722, in its proximal unfired position defats the firing drive lock 40705 so the firing driver 40710 can perform another firing and retraction stroke.

If the spent staple cartridge 40720 with the sled 40722 in the position of FIG. 133 is removed from the end effector 40730 and placed in another surgical stapling instrument identical to the surgical stapling instrument 40700, the firing driver 40710 will not be able to perform a firing stroke. Specifically, when the sled 40722 is in the position of FIG. 133, the distal nose 40716 of the firing driver 40710 will not be able to engage and rest on the ledge 40728 of the sled 40722 when the firing driver 40710 is distally advanced and, thus, the first cam 40711 of the firing driver 40710 will be biased into the lockout opening 40739 preventing the firing driver 40710 from performing the firing stroke through the spent, or at least partially spent, staple cartridge 40720. Moreover, the first cam 40711 positioned in the lockout opening 40739 is viewable to a user of the attachment 40700 and indicates that the firing driver 40710 is locked out and prevented from performing the firing stroke.

Further to the above, in at least one instance, the latch arm 40718 of the firing driver 40710 comprises a unique keyed geometry that can only be received by a complementary keyed relief feature (cavity) in the sled 40722. As such, if a non-compatible staple cartridge is inserted into the end effector 40730, the arm latch 40718 of the firing driver 40710 will not be able to properly engage with the sled of the non-compatible staple cartridge. In such instances, the latch arm 40718 would be prevented from advancing up the proximal ramp portion 40737 and thereby prevent the distal advancement of the firing driver 40710.

As can also be seen in FIGS. 134 and 135, the staple cartridge 40720 further comprises a flexible tab 40725 that is configured to retain the sled 40722 in the proximal unfired position during shipping and during installation into the end effector 40730. FIG. 134 illustrates the position of the sled 40722 in an unfired state. This is the state in which the staple cartridge 40720 is shipped and stored prior to use. As can be seen in FIG. 134, the flexible tab 40725 frictionally engages a corresponding rail 40729 of the sled 40722 and serves to prevent the sled from moving out of that unfired position prior to use. In at least one instance, the staple cartridge 40720 comprises another flexible tab 40725 on the other side of a longitudinal slot 40721 of the staple cartridge 40720 that is configured to engage another rail 40729 of the sled 40722. In any event, the flexible tab, or tabs 40725, are biased toward the first jaw 40731 to engage the sled 40722 to prevent the sled 40722 from inadvertently moving prior to firing the staple cartridge 40720.

In use, as the firing driver 40710 engages the sled 40722 during the firing stroke, the flexible tab 40725 rides up the rail 40729 until the sled 40722 moves distally out of the unfired position and the flexible tab 40725 completely disengages the rail 40729. After the firing stroke has been completed, the sled 40722 is retracted proximally by the firing driver 40710 until the rails 40729 engage the flexible tab 40725 as shown in FIG. 135. As such, when the sled 40722 is retracted during the retraction stroke, the flexible tab 40725 prevents the sled 40722 from returning to its original proximal unfired position (i.e., the position of the sled 40722 in FIG. 134). Specifically, a hook portion 40725a of the flexible tab 40725 engages a proximal face 40723 of the sled 40722 to prevent further proximal retraction of the sled 40722. This position may coincide with the released positioned discussed above, for example. As was further discussed above, if the sled 40722 is not in its proximal unfired position, the firing driver lock 40705 will not be defeated and the firing driver 40710 will be unable to perform a firing stroke.

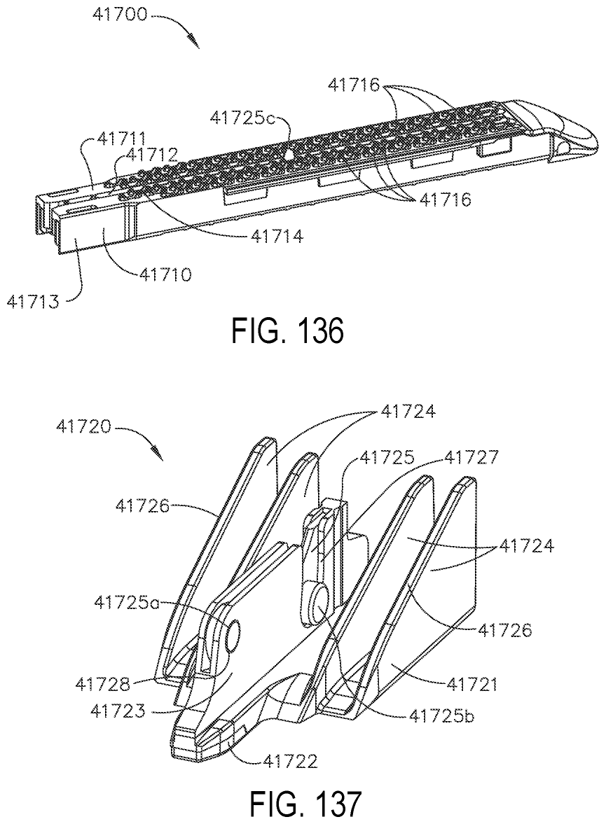

FIGS. 136-144 illustrate a staple cartridge 41700 that comprises a cartridge body 41710 and a sled 41720 that is movable within the cartridge body 41710. The cartridge body 41710 comprises a longitudinal slot 41712, a deck 41714, and a plurality of longitudinal rows of staple cavities 41716 defined in the deck 41714. The longitudinal slot 41712 divides the staple cartridge 41700 into a first side 41711 and a second side 41713 opposite the first side. The first side 41711 and the second side 41713 are connected at the distal end of the cartridge body 41710. Three rows of staple cavities 41716 are positioned on the first side 41711 of a longitudinal slot 41712 and three rows of staple cavities 41716 are positioned on a second side of the longitudinal slot 41712. However, other staple cavity arrangements are envisioned. The staple cartridge 41700 further comprise a plurality of staple drivers movably positioned within the staple cavities 41716. The staple drivers support staples thereon for ejection from the staple cartridge 41700.

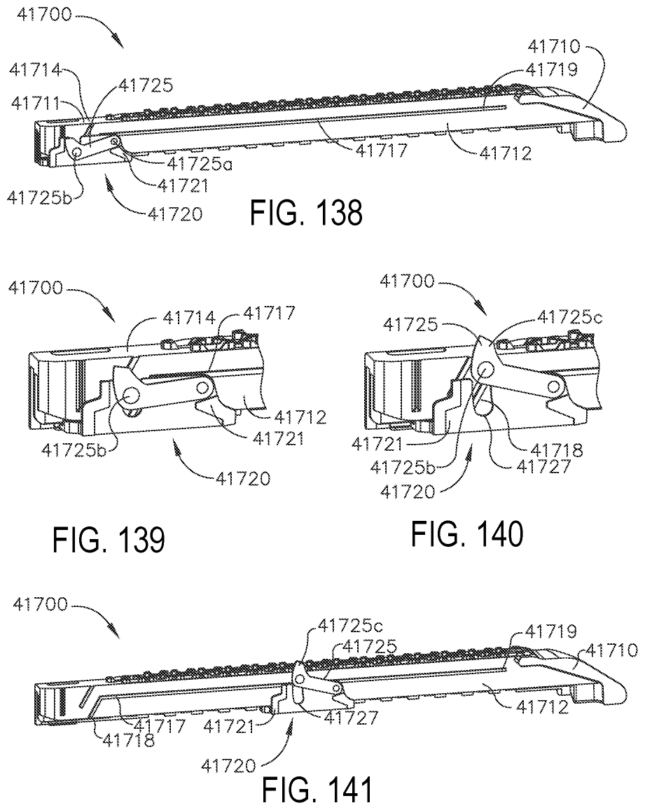

As can be seen in FIG. 138, the cartridge body 41710 comprises a ledge 41717 extending laterally into the longitudinal slot 41712 from the first side 41711 of the cartridge body 41710. In at least one instance, the cartridge body 41710 comprises another ledge extending into the longitudinal slot 41712 from the second side 41713 of the cartridge body 41710 in line with the ledge 41717. In any event, the ledge 41717 extends longitudinally within the longitudinal slot 41712 and comprises a proximal ramp 41718 and a distal end 41719 as depicted in FIG. 141.

Referring now to FIG. 137, the sled 41720 comprises a wedge portion 41721 and a knife 41725 that is rotatable relative to the wedge portion 41721. The wedge portion 41721 comprises a base 41722, a central nose portion 41723 extending upward from the base 41722, and a plurality of rails 41724 extending upward from the base 41722 on either side of the nose portion 41723. Two rails 41724 are positioned on one side of the base 41722 and two rails 41724 are positioned on the opposite side of the base 41722. Each of the rails 41724 comprises a distal ramp 41726 configured to cammingly engage the staple drivers in the cartridge body 41710 to eject the staples from the staple cavities 41716 when the sled 41720 is moved from a proximal unfired position (FIG. 138) to a distal fired position (FIG. 143) during a firing stroke. Further, the central nose portion 41723 is configured to move within the longitudinal slot 41712 of the cartridge body 41710 during the firing stroke.

As can be seen in FIG. 137, a proximal slot 41727 and a distal through hole 41728 is provided in the nose portion 41723. The knife 41725 is pivotably connected to the wedge portion 41721 by a distal pin 41725a that is received within the distal through hole 41728 in the nose portion 41723. As can be further seen in FIG. 137, the knife portion 41725 comprises a proximal pin 41725b that is movably received within the proximal slot 41727 in the nose portion 41723. The proximal pin 41725b is configured to interact with the ledge portion 41717 in the cartridge boy 41710 when the sled 41720 moves therethrough.

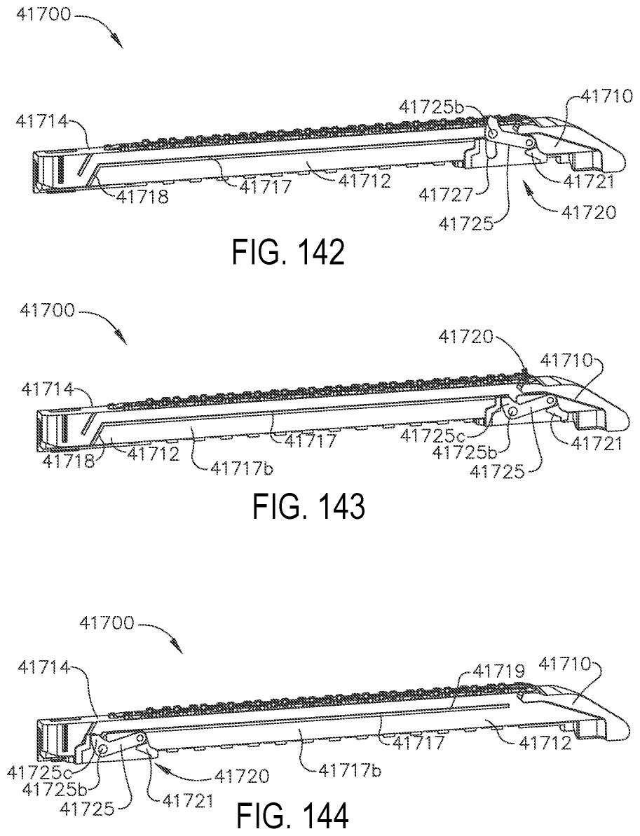

When the staple cartridge 41700 is initially inserted into an end effector of a surgical stapling instrument, the sled 41720 is in a proximal unfired position illustrated in FIG. 138. Specifically, the wedge portion 41721 of the sled 41720 is in a proximal unfired position and the knife 41725 is in a retracted position. When the sled 41720 is advanced from the position in FIG. 138 to the position in FIG. 139, the proximal pin 41725b engages the proximal ramp 41718 of the ledge 41717 in the cartridge body to lift the knife 41725 within the proximal slot 41727 of the wedge portion 41721 toward the deck 41714. Further distal advancement of the sled 41720 from the position in FIG. 139 to the position in FIG. 140, lifts the knife 41725 into a position wherein a cutting portion of the knife protrudes out of the longitudinal slot 41712. For example, when the knife 41725 is in the exposed configuration depicted in FIG. 140, a knife edge 41725c of the knife 41725 is at least partially positioned above the deck 41714 of the cartridge body 41710. Further, the proximal pin 41725b is configured to ride upon the ledge 41717 in the longitudinal slot 41712 to maintain the knife 41725 in the exposed configuration as the sled 41720 is further distally advanced from the position in FIG. 140 to the position in FIG. 142. When the sled 41720 moves from the positon in FIG. 142 to a distal fired position illustrated in FIG. 143, the proximal pin 41725b falls from the distal end 41719 of the ledge 41717 and into the proximal slot 41727 of the central nose portion 41723. As such, the knife 41275 moves from the exposed configuration in FIG. 142 into the retracted configuration illustrated in FIG. 143. In one embodiment, the sled 41720 is permitted to remain in the distal fired position within the staple cartridge 41700 (FIG. 143) and is not retracted back to the proximal end of the staple cartridge. The staple cartridge can then be removed from the end effector with the knife 41725 in a retracted, or non-exposed, configuration and, thus, the possibility of a user coming into contact with the knife 41549 while handling the spent staple cartridge 41700 is reduced.

In at least one alternative arrangement, the sled 41720 may be configured in the various manners discussed herein to enable the sled 41720 to otherwise be retracted from the distal fired position (FIG. 143) to a proximal position illustrated in FIG. 144. In such instances, the proximal pin 41725b is maintained in the proximal slot 41727 of the sled 41720 on an underside 41717b of the ledge 41717 during retraction. The ledge 41717 prevents the knife 41725 from becoming exposed above the deck 41714 of the cartridge body 41710 as the sled 41720 is retracted and when the sled is in the proximal position illustrated in FIG. 144. As such, the staple cartridge 41700 can be removed from the end effector with the knife 41725 in a retracted, or non-exposed, configuration and the sled 41720 in the proximal position illustrated in FIG. 144. In addition, in the event the sled 41720 is only partially distally advanced during the firing stroke, such as to the position illustrated in FIG. 141, the firing driver 40710 can be employed to retract the sled 41720 proximally into the position illustrated in FIG. 138 thereby enabling the partially fired staple cartridge 41700 to be removed from the end effector with the knife 41725 in a retracted, or non-exposed position.

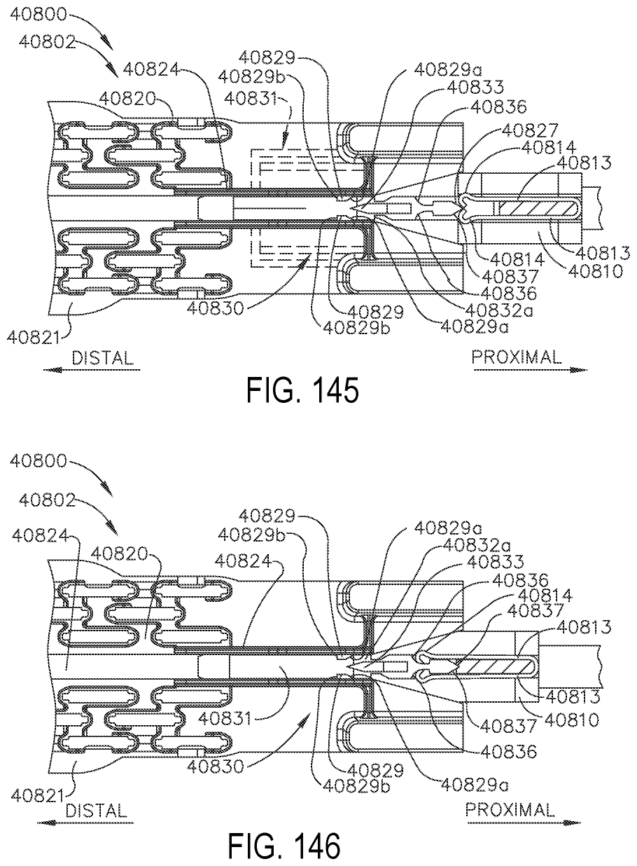

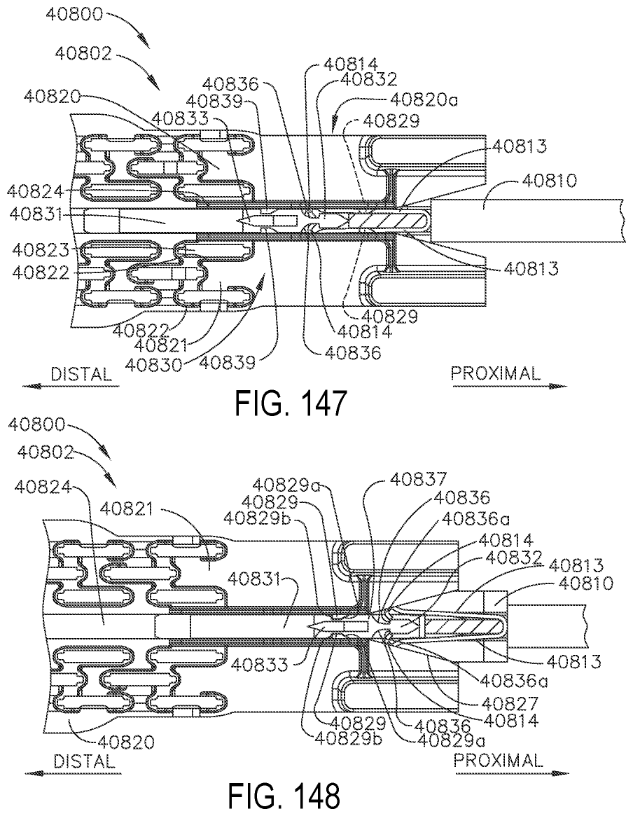

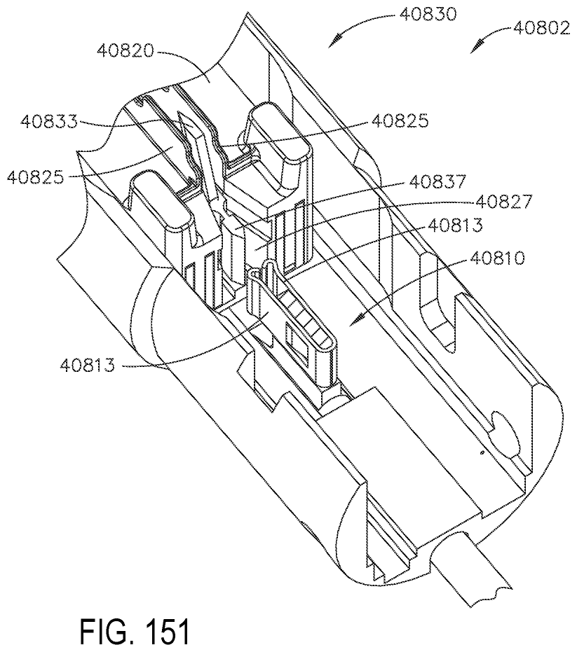

FIGS. 145-151 illustrate a portion of a surgical system 40800 comprising an end effector 40802 configured to receive a staple cartridge 40820. The surgical system 40800 further comprises a firing driver, sled driver or firing actuator 40810 that is movable relative to the end effector 40802. The end effector 40802 comprises a first jaw configured to receive the staple cartridge 40820 and a second jaw movable relative to the first jaw to capture tissue between the first jaw and the second jaw similar to the end effector depicted in FIG. 129, for example. The staple cartridge 40820 may be similar to the staple cartridge 40720 except for the differences noted herein. For example, in the illustrated arrangement, the staple cartridge 40820 comprises a cartridge body 40820a that defines a deck surface 40821. A plurality of staple cavities 40822 are defined in the deck surface 40821. A longitudinal slot 40824 is defined in the deck surface 40821 and divides the staple cartridge 40820 into a first side and a second side opposite the first side. As can be seen in FIG. 151, a proximal portion of the longitudinal slot 40824 comprises a V-cutout portion 40827 that opens through the proximal end of the staple cartridge 40820. As can be seen in FIGS. 145 and 146, a cartridge slot protrusion 40829 protrudes inwardly from each side of the longitudinal slot 40824. Each cartridge slot protrusion 40829 comprises a proximal face 40829a and a distal face 40829b. The proximal faces 40829a are ramped, or tapered, laterally and each of the distal faces 40829b are laterally flat, or substantially flat.

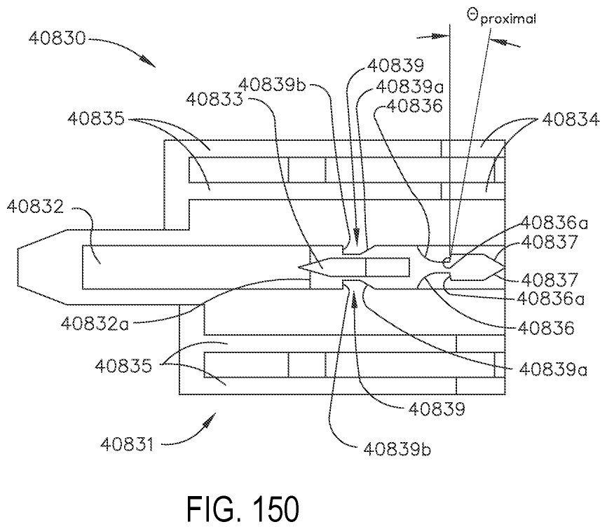

Turning to FIG. 150, the staple cartridge 40820 includes a sled 40830 that comprises a wedge portion 40831 and a nose portion 40832. The wedge portion 40831 comprises a plurality of rails 40834. Each of the rails 40834 comprises a distal ramp 40835 that is configured to cammingly engage staple drivers 40823 (see FIG. 146) to eject staples or fasteners supported on the staple drivers 40823 from the staple cartridge 40820. The nose portion 40832 of the sled 40830 comprises a knife 40833 that is configured to extend above the deck surface 40821. The knife 40833 is configured to incise patient tissue that is captured between the first jaw and the second jaw of the end effector 40802. Further, when the sled 40830 is in a proximal unfired position, the knife 40833 is configured to be stored within a knife housing 40825 formed in the cartridge body. The knife housing 40825 extends above the deck surface 40821 as depicted in FIG. 151.

As can be seen in FIG. 150, the nose portion 40832 of the sled 40830 further comprises proximal recesses 40836, distal recesses 40839, and proximal ramps 40837. Each of the distal recesses 40839 comprises a proximal ramp 40839a and a distal wall 40839b. Further, each of the proximal recesses 40836 comprises a proximal wall 40836a tapered at angle θₚᵣₒₓᵢₘₐₗ. In any event, the proximal ramps 40837 and the proximal recesses 40836 and are configured to permit the firing driver 40810 to selectively engage and disengage the sled 40830, as discussed in greater detail below.

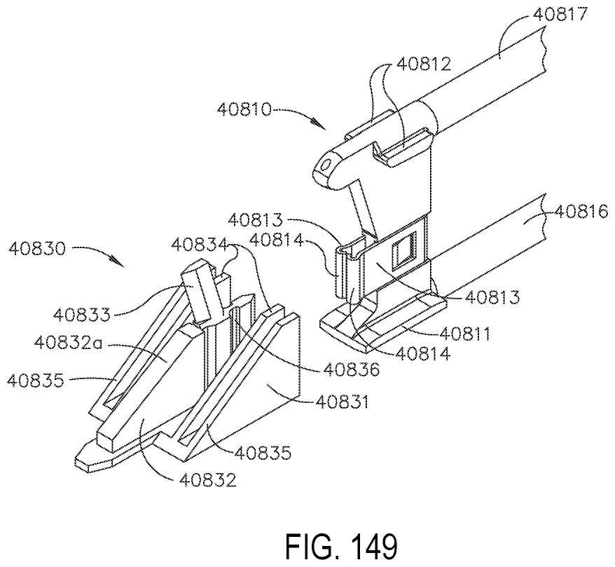

Referring primarily to FIG. 149, the firing driver 40810 comprises a first lateral cam 40811 configured to engage the first jaw of the end effector 40802 and second lateral cams 40812 configured to engage the second jaw of the end effector 40802. In one arrangement, the first and second lateral cams 40811, 40812 are configured to maintain the positioning of the first and second jaws of the end effector 40802 relative to one another during firing and retraction strokes of the firing driver 40810. As can be seen in FIG. 149, the firing driver 40810 further comprises a first firing bar, rod, element, driver assembly, etc. 40186 extending proximally from the firing driver 40810 and a second firing bar, rod, element, driver assembly, etc. 40817 extending proximally from the firing driver 40810. The first firing bar 40816 is spaced apart from the second firing bar 40817. The first and second firing bars 40816, 40817 are configured to transfer a firing motion from the surgical system 40800 to the firing driver 40810 to translate the firing driver 40810 distally and proximally. Further, the firing driver 40810 comprises a pair of opposing lateral spring arms 40813 extending distally from the firing driver 40810. The lateral spring arms 40813 are biased toward one another and are selectively engageable with the sled 40830 of the staple cartridge 40820, as discussed in greater detail below.

In use, the staple cartridge 40820 is inserted into the end effector 40802 with the sled 40830 in a proximal unfired position depicted in FIG. 145. The firing driver 40810 is initially distally advanced from a starting position toward the staple cartridge 40820 causing the lateral spring arms 40813 of the firing driver 40810 to engage the proximal ramps 40837 of the sled 40830 and be biased away from one another as depicted in FIG. 146. Further distal advancement of the firing driver 40810 results in hooked ends 40814 of the spring arms 40813 snapping into the proximal recesses 40836 of the nose portion 40832 of the sled 40830 to operably couple the firing driver 40810 to the sled 40830 as depicted in FIG. 146. In some instances, the hooked ends 40814 of the spring arms 40813 may engage the v-cutout portion 40827 of the longitudinal slot 40824 of the staple cartridge 40820 to guide the spring arms 40813 toward the proximal recesses 40836 of the sled 40830 to aid in coupling the firing driver 40810 to the sled 40830. Further, in at least one instance, a cartridge body feature, such as the flexible tab 40725 (see FIG. 134) is configured to hold the sled 40830 in the proximal unfired position depicted in FIGS. 145 and 146. The sled 40830 is held stationary by the flexible tab 40725 to permit the spring arms 40813 to biased away from one another as the firing driver 40810 is advanced toward the sled 40830 to operably couple with the sled 40830. When the sled 40830 is advanced from the proximal unfired position in FIG. 145 to a distal fired position by the firing driver 40810 during the firing stroke, the biasing force of the flexible tab 40725 is overcome to permit the sled 40830 to be distally advanced beyond the proximal unfired position.

Further to the above, when the sled 40830 is in the proximal unfired position depicted in FIG. 145, the nose portion 40832 of the sled 40830 is at least partially positioned proximal to the cartridge slot protrusions 40829. Specifically, a distal ramp 40832a of the nose portion 40832 is positioned proximal to the cartridge protrusions 40829. In use, the sled 40830 is advanced by the firing driver 40810 such that the distal ramp 40832a of the nose portion 40832 of the sled 40830 engages the proximal ramps 40829a of the cartridge slot protrusions 40829 to permit the sled 40830 to pass by the cartridge slot protrusions 40829. In other words, the firing driver 40810 is configured to apply enough force to the sled 40830 to permit the sled 40830 to bypass the cartridge protrusions 40829 owing to the proximal ramps 40829a of the cartridge slot protrusions 40829.

Further to the above, the hooked ends 40814 of the spring arms 40813 are retained in their corresponding proximal recesses 40836 of the sled 40830 by the longitudinal slot 40824 as the sled 40830 is advanced and retracted through the staple cartridge 40820. In other words, the walls of the staple cartridge 40820 defining the longitudinal slot 40824 prevent the lateral spring arms 40813 from disengaging from the proximal recesses 40836 of the sled 40830 as long as the proximal recesses 40836 are positioned within the longitudinal slot 40824 with the spring arms 40813 positioned therein. Further, the hooked ends 40814 of the spring arms 40813 are configured to engage the proximal wall 40836a of the proximal recess 40836 of the sled 40830 to permit the firing driver 40810 to retract, i.e., pull, the sled 40830 proximally. As such, the firing driver 40810 is configured to advance the sled 40830 from the proximal unfired position (FIG. 145) to a distal fired position during the firing stroke to enable the wedge portion 40831 to eject the staples from the staple cartridge 40820 into patient tissue and the knife 40833 of the nose portion 40832 to incise the stapled patient tissue. Further, the firing driver 40810 is configured to retract the sled 40830 from the distal fired position to a proximal returned position (FIG. 148) during a retraction stroke owing to the engagement of the hooked ends 40814 of the spring arms 40813 with the proximal recesses 40836 of the sled 40830. As the sled 40830 approaches the proximal returned position, the distal wall 40839b of the distal recesses 40839 of the sled 40830 engage the distal faces 40829b of the cartridge slot protrusions 40829 of the staple cartridge 40820 to prevent the sled 40830 from moving further proximally from the proximal returned position in FIG. 148. Once the sled 40830 attains the proximal returned position, further proximal retraction of the firing driver 40810 causes the lateral spring arms 40813 of the firing driver 40810 to splay and disengage the proximal recesses 40836 of the sled 40830. Specifically, the proximal walls 40836a of the proximal recesses 40836 tapered at angle θₚᵣₒₓᵢₘₐₗ permit the hooked ends 40814 of the spring arms 40813 to disengage from the proximal recesses 40836 of the sled 40830 and, thus, the firing driver 40810 and the sled 40830 are operably decoupled. Further, the v-cutout portion 40827 of the longitudinal slot provides space for the hooked ends 40814 of the lateral spring arms 40813 to move away from each other and out of the proximal recesses 40836 of the sled 40830.

Further to the above, when the firing driver 40810 is operably decoupled from the sled 40830 as depicted in FIG. 148, the sled 40830 remains in the proximal returned or leased positioned within the staple cartridge 40820. As such, the original proximal unfired position of the sled 40830 (FIGS. 145 and 146) is different than the return proximal position of the sled 40830 depicted in FIG. 148. Specifically, the returned or released position of the sled 40830 depicted in FIG. 148 is distal to the original proximal unfired position of the sled 40830 in FIG. 145. As such, when the staple cartridge 40820 was initially inserted into the end effector 40802 with the sled 40830 in the proximal unfired position, a firing driver lockout, such as those discussed above, is defeated to permit the firing driver 40810 to perform the firing stroke. However, when the sled 40830 is returned to the proximal returned or released position depicted in FIG. 148, the sled 40830 is distal to the original proximal unfired position and therefore can no longer defeat the firing lockout. As such, the firing driver 40810 is not permitted to perform another firing stroke when the sled 40830 is in the proximal returned position.

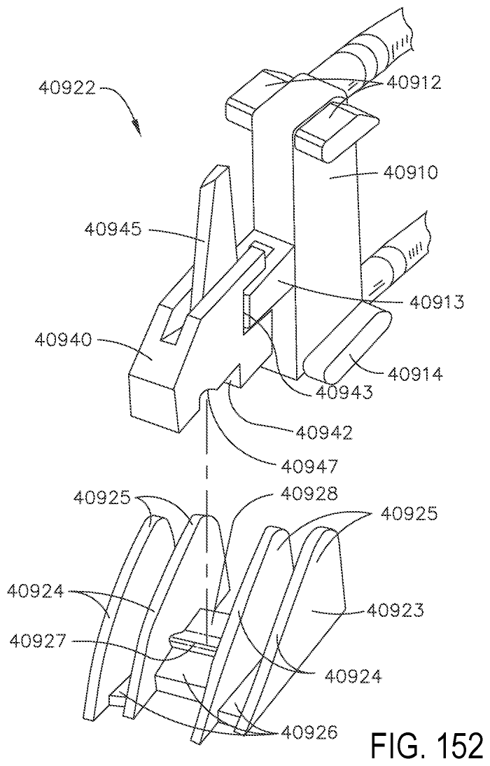

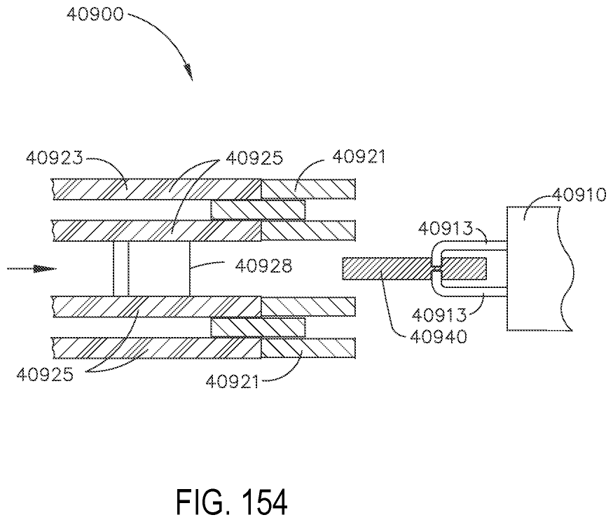

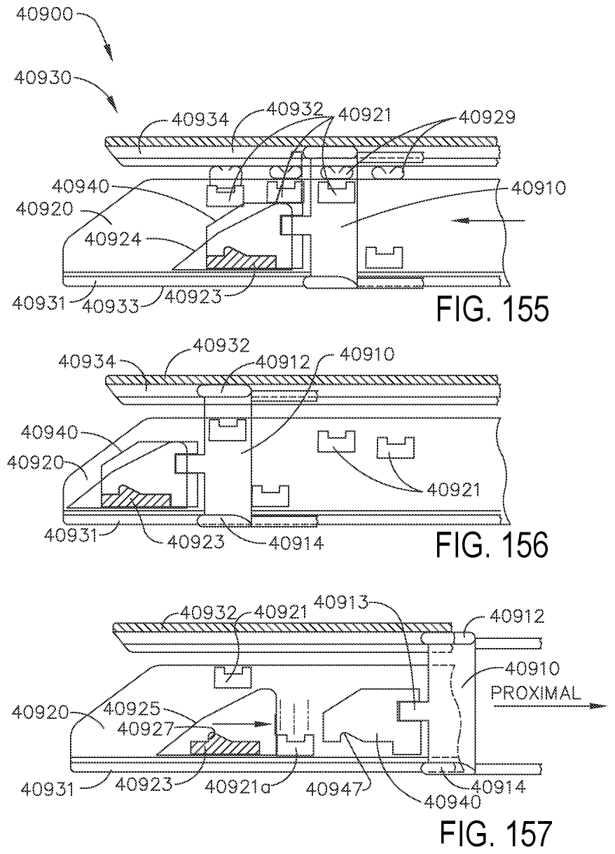

FIGS. 152-157 illustrate a surgical instrument 40900 comprising an end effector 40930 configured to receive a staple cartridge 40920 and a firing driver 40910. The staple cartridge 40920 is replaceable, i.e., the staple cartridge 40920 is readily removable from the end effector 40930 during a surgical procedure and replaceable with another staple cartridge; however, embodiments are envisioned in which the staple cartridge 40920 is not replaceable. The end effector 40930 comprises a first jaw 40931 and a second jaw 40932 movable relative to the first jaw 40931 to capture tissue in between the first jaw 40931 and the second jaw 40932. However, in at least one embodiment, the first jaw 40931 is movable relative to the second jaw 40932. The staple cartridge 40920 comprises a plurality of staple drivers 40921 and a sled assembly 40922 movable distally to eject staples 40929 from the staple cartridge 40920 during a firing stroke of the firing driver 40910. Referring to FIG. 154, the staple drivers 40921 are triple staple drivers configured to support three staples thereon. Further, the staple drivers 40921 are positioned on both sides of a longitudinal slot defined in the staple cartridge 40920 and are configured to eject staples 40929 from a plurality of longitudinal rows of staple cavities defined in the staple cartridge 40920.

Further to the above, the firing driver 40910 comprises a first lateral cam 40914 configured to engage the first jaw 40931 and a second lateral cam 40912 configured to engage the second jaw 40932. The first jaw 40931 comprises a longitudinal cavity 40933 defined therein that is configured to receive the first cam 40914 of the firing driver 40910. Further, the second jaw 40932 comprises a longitudinal cavity 40934 defined therein that is configured to receive the second cam 40912 of the firing driver 40610. During the firing stroke, the first cam 40914 and the second cam 40912 are configured to maintain the position of the first jaw 40931 relative to the second jaw 40932.

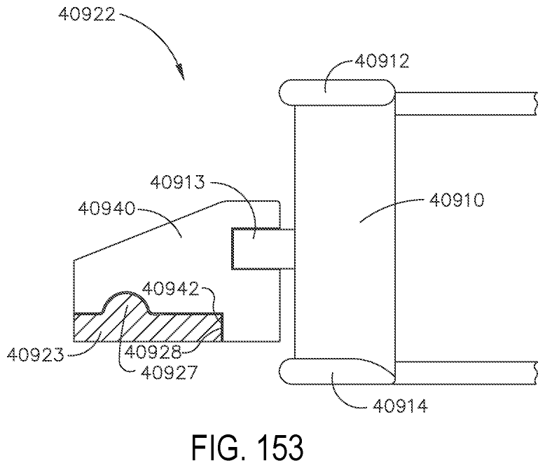

Further to the above, referring to FIGS. 152 and 153, the sled assembly 40922 comprises a wedge portion 40923 and a knife portion 40940. The wedge portion 40923 comprises a plurality of rails 40925 interconnected by a base 40926. Each of the rails 40925 comprises a distal ramp 40924 facing distally that is configured to cammingly engage the staple drivers 40921 of the staple cartridge 40920 to eject the staples 49023 from the staple cartridge 40920 during the firing stroke. The knife portion 40940 is positioned on and connected to the wedge portion 40923 as illustrated in FIG. 153. Specifically, the knife portion 40940 comprises a cutout 40947 defining a distal wall 40942 and the base portion 40926 of the wedge portion 40923 comprises a central protrusion 40927 received in the cutout 40947 of the knife portion 40940 as illustrated in FIG. 153. The cutout 40947 of the knife portion 40940 is sized and shaped to receive the complementary central protrusion 40927 of the sled 40922. The cutout 40947 of the knife portion 40940 and the central protrusion 40927 of the sled 40922 releasably interlock the knife portion 40940 and the wedge portion 40923 together, as discussed in greater detail further below.

In use, the staple cartridge 40920 is seated in the first jaw 40931 of the end effector 40930 with the sled assembly 40922 positioned in a proximal unfired position and the firing driver 40910 positioned proximal to the sled assembly 40922. The firing driver 40910 comprises opposing arms 40913 that each comprises a resilient cantilever configured to flex laterally outwardly and resiliently return laterally inwardly. In at least one embodiment, the arms 40913 are part of a metal clip, for example. At the outset of the firing stroke, the firing driver 40910 is advanced distally toward the sled assembly 40922 such that the arms 40913 engage and attach the firing driver 40910 to the knife portion 40940 of the sled assembly 40922. More specifically, each of the arms 40913 comprises a shoulder that snap-fits into a recess 40943 defined in the proximal end of the knife portion 40940 during the firing stroke of the firing driver 40910. That said, the firing driver 40910 can be configured to connect to the knife portion 40940 in any suitable manner. As the knife portion 40940 of the sled assembly 40922 is pushed distally by the firing driver 40910, the distal wall 40942 of the knife portion 40940 abuts a proximal wall 40928 of the wedge portion 40923 (see FIG. 153) such that the firing driver 40910 advances the wedge portion 40923 and the knife portion 40940 together distally. During the firing stroke, the distal ramps 40924 of the sled 40922 engage and move the staple drivers 40921 from unfired positions to fired positions to eject the staples 40929 from the staple cartridge 40920. After the staple firing stroke has been completed, or at least partially completed, the firing driver 40910 is pulled proximally to retract the firing driver 40910 and the sled assembly 40922 to their proximal unfired positions. When the now-spent staple cartridge 40920 is removed from the end effector, the sled assembly 40922 detaches from the firing driver 40910. At such point, a new, or unspent staple cartridge, can be seated in the end effector.

In various instances, further to the above, the sled assembly 40922 may become stuck and may not be retractable after the firing stroke. For instance, one or more of the drivers 40921 may fall from their fired position toward their unfired position behind the sled assembly 40922 after the sled assembly 40922 has passed thereby during the firing stroke. FIG. 157 illustrates a staple driver 40921a of the staple drivers 40921 which is in its unfired position after the firing stroke has been completed which could block the sled assembly 40922 from being retracted. Absent more, the tissue cutting knife 40945 would be exposed if the end effector were to be re-opened at such point. To address this potential issue, the knife portion 40940 of the sled assembly 40922 can detach from the wedge portion 40923 when the sled assembly 40922 is being retracted if the wedge portion 40923 gets stuck or blocked by a staple driver 40921, such as the staple driver 40921a, for example. When the firing driver 40910 is retracted with enough force, the engagement between the rails 40925 and the driver 40921a causes the central protrusion 40927 of the wedge portion 40923 to move out of the cutout 40947 of the knife portion 40940 to separate the wedge portion 40923 and the knife portion 40940. The knife portion 40940 is then retracted proximally by the firing driver 40910—leaving behind the wedge portion 40923—to complete the retraction stroke. As such, in the event one or more staple drivers 40921 are in the path of the rails 40925 of the sled assembly 40922 when the sled assembly 40922 is retracted, for instance, the knife 40945 and the firing driver 40910 can still be retracted to their proximal unfired positions.

Further to the above, in at least one instance, the wedge portion 40923 comprises a first material and the knife portion 40940 comprises a second material that is different than the first material. For example, the wedge portion 40923 is comprised of plastic and the knife portion 40940 is comprised of metal. In at least one embodiment, the wedge portion 40923 is comprised of polyvinylchloride, for example, and the blade of the knife portion 40940 is comprised of stainless steel, for example. In at least one embodiment, the knife portion 40940 comprises a plastic material and the wedge portion 40923 comprises a metal material.

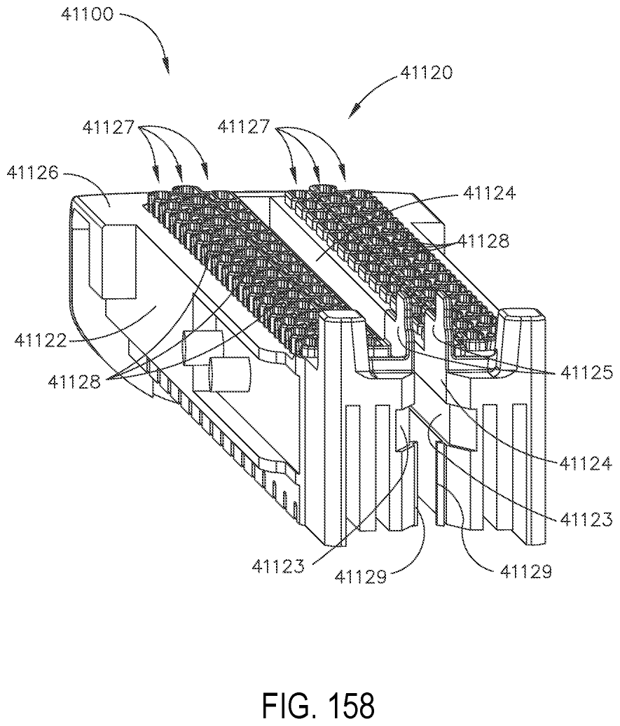

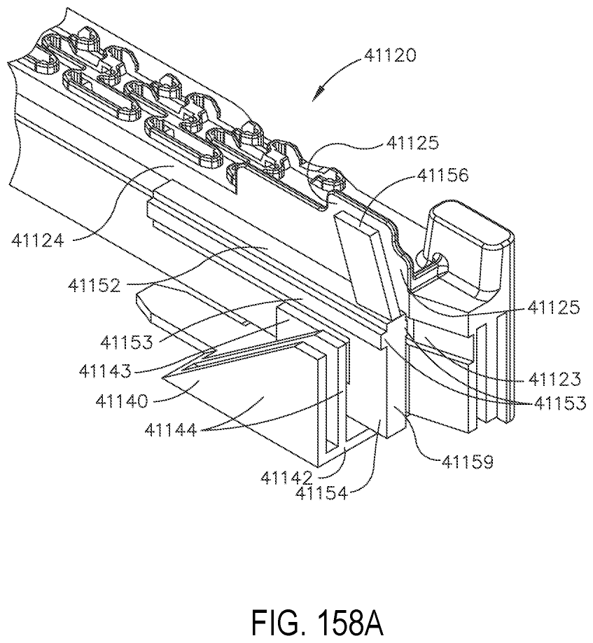

FIGS. 158-160 illustrate a stapling system 41100 comprising an end effector configured to receive a staple cartridge 41120. The staple cartridge 41120 is replaceable; however, other embodiments are envisioned in which the staple cartridge 41120 is not replaceable. The staple cartridge 41120 comprises a cartridge body 41122 and a sled assembly 41130 movable within the cartridge body 41122. The cartridge body 41122 comprises a deck 41126, a longitudinal slot 41124, and a plurality of longitudinal rows of staple cavities 41127 defined in the deck 41126. Three rows of staple cavities 41127 are positioned on a first side of the longitudinal slot 41124 and three rows of staple cavities 41127 are positioned on a second side of the longitudinal slot 41124. Each staple cavity 41127 comprises at least one staple cavity extender 41128 extending from the deck 41126 that at least partially surrounds the staple cavity 41127. That said, other embodiments are envisioned in which only some of the staple cavities 41127 have staple cavity extenders 41128 while other embodiments are envisioned in which none of the staple cavities 41127 have staple cavity extenders. The staple cartridge 41120 further comprise a plurality of staple drivers movably positioned within the staple cavities 41127 that support staples thereon. Further, the staple cartridge 41120 comprises a longitudinal recess 41123 defined in the body portion 41122 on each side of the longitudinal slot 41124, a cartridge slot protrusion 41129 extending into the longitudinal slot 41124 on each side of the longitudinal slot 41124 at the proximal end thereof, and a knife housing portion 41125 extending upwardly from the deck 41126 at the proximal end of the longitudinal slot 41124. The longitudinal recesses 41123 and the cartridge slot protrusions 41129 are configured to interact with the sled assembly 41130, as discussed in greater detail below.

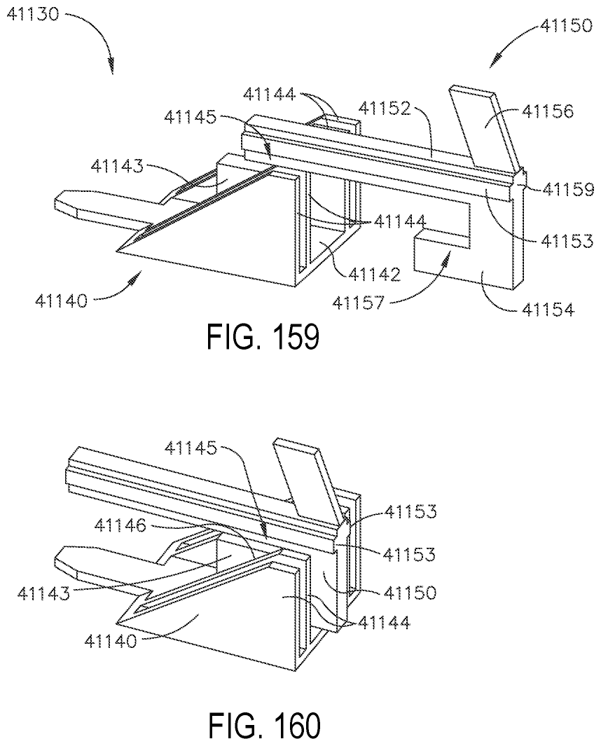

Referring to FIGS. 159 and 160, the sled assembly 41130 comprises a wedge portion 41140 and a knife portion 41150. The wedge portion 41140 comprises a base 41142, a central portion 41143, and a plurality of rails 41144 extending from the base 41142. The central portion 41143 comprises a proximal protrusion 41145 extending proximally from the central portion 41143 wherein two of the rails 41144 are positioned on each side of the central portion 41143. Each of the rails 41144 comprises a distal ramp 41146 facing distally. The distal ramps 41146 are configured to cammingly engage the staple drivers positioned in the cartridge body 41122 to eject the staples from the staple cartridge 41120. The knife portion 41150 comprises a longitudinal nose portion 41152, a hook portion 41154 extending below the nose portion 41152, and a knife, or blade, 41156 extending above the nose portion 41152. The hook portion 41154 defines an opening 41157 in the knife portion 41150 which, as discussed in greater detail below, is configured to receive a portion of the wedge portion 41140 therein.

Further to the above, a firing driver, such as the firing driver 40910 discussed above, for example, engages the knife portion 41150 of the sled assembly 41130 during a firing stroke to advance the knife portion 41150 and the wedge portion 41140 together from a proximal unfired position to a distal fired position. As the knife portion 41150 is pushed distally by the firing driver, the knife portion 41150 pushes the wedge portion 41140 distally owing to the engagement of the hook portion 41154 of the knife portion 41150 with the proximal protrusion 41145 of the wedge portion 41140. After the firing stroke has been completed, or at least partially completed, the firing driver can be retracted so that the jaws of the stapling system 41100 can be re-opened and/or so that the stapling system 41100 can be reset. In such instances, the firing driver retracts the knife portion 41150 proximally to its original proximal unfired position within the staple cartridge 41120. The firing driver and/or the knife portion 41150 comprise one or more features that couple the firing driver and knife portion 41150 so that the firing driver can retract the knife portion 41150. The wedge portion 41140, however, remains in the distal fired position within the staple cartridge 41120 and is not retracted with the knife portion 41150. The interface between the knife portion 41150 and the wedge portion 41140 permits the knife portion 41150 to slide proximally away from the wedge portion 41140 as the knife portion 41150 is being retracted.

Further to the above, the knife portion 41150 can rest upon and/or can be supported by the wedge portion 41140 during the staple firing stroke. When the knife portion 41150 is retracted proximally relative to the wedge portion 41140 during the retraction stroke, however, the knife portion 41150 can no longer be supported by the wedge portion 41140. That being said, the cartridge body 41122 is configured to support the knife portion 41150 during the staple firing stroke and the retraction stroke. More specifically, the knife portion 41150 comprises lateral protrusions 41153 that are received within and are supported by the sidewalls of longitudinal recesses 41123 defined in the cartridge body 41122 during the staple firing and retraction strokes. The lateral protrusions 41153 extend longitudinally along the lateral sides of the nose portion 41152 and slide within the longitudinal recesses 41123. Owing to the configuration of the lateral protrusions 41153 and the longitudinal recesses 41123, the knife portion 41150 is prevented from, or at least limited in, moving vertically within the longitudinal slot 41124 of the cartridge body 41122. In any event, the knife, or blade, 41156 of the knife portion 41150 is housed within the knife housing portion 41125 of the cartridge body 41122 when the knife portion 41150 is in its proximal unfired position.

When the knife portion 41150 is retracted into its proximal unfired position by the firing driver, a proximal end 41159 of the knife portion 41150 engages the cartridge slot protrusions 41129 of the cartridge body 41122 which stops the proximal movement of the knife portion 41150. The firing driver is then retracted further proximally into its unfired position which causes the firing driver to detach from the knife portion 41150. As such, the sled assembly 41130 of the staple cartridge 41120 is not attached to the firing driver of the stapling system 41100 when the staple cartridge 41120 is removed from the stapling system 41100. Moreover, the knife, or blade, 41156 of the knife portion 41150 is stored or stowed within the knife housing 41125 of the cartridge body 41122 so that the tissue cutting edge of the knife 41156 is not accidentally touched by a clinician handling the staple cartridge 41120.

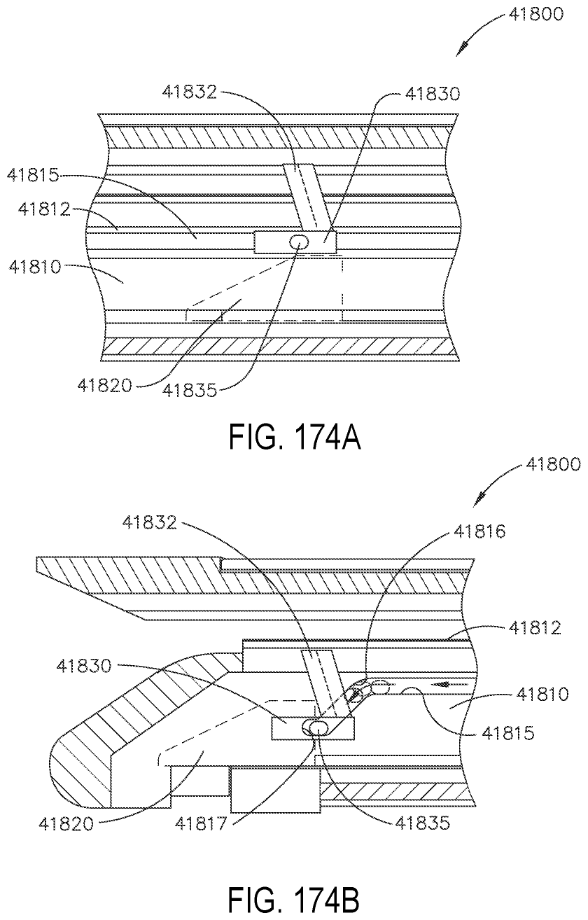

A staple cartridge 41800 is depicted in FIGS. 174A and 174B that is usable with a surgical stapling instrument comprising a firing driver. The staple cartridge 41800 comprises a cartridge body 41810 including a deck 41812 configured to support patient tissue and staple cavities defined in the deck 41812, staple drivers movable within the staple cavities, and staples removably stored in the staple cavities that are driven by the staple drivers during a staple firing stroke. The staple cartridge 41800 further comprises a sled 41820 moveable from a proximal unfired position to a distal fired position (FIG. 174B) by the firing driver during the staple firing stroke. Similar to the other sleds disclosed herein, the sled 41820 engages the staple drivers during the staple firing stroke to eject the staples from the staple cartridge 41800 and deform the staples against an anvil positioned opposite the staple cartridge 41800. The staple cartridge 41800 further comprises a tissue cutting knife 41830 that is also pushed distally by the firing driver during the staple firing stroke. Notably, the tissue cutting knife 41830 is not connected to the sled 41820 but it does move with the sled 41820 during the staple firing stroke. During the staple firing stroke, referring to FIG. 174A, a knife blade 41832 of the tissue cutting knife 41830 extends above the deck 41812 of the cartridge body 41810 such that the knife blade 41832 incises patient tissue positioned intermediate the anvil and the deck 41812 of the staple cartridge 41800. At the end of the staple firing stroke, referring to FIG. 174B, the tissue cutting knife 41830 descends, or dives, downwardly within the cartridge body 41810 such that the knife blade 41832 is positioned below the top surface of the deck 41812. Such downward motion of the tissue cutting knife 41830 is controlled by one or more cams defined on the cartridge body. More specifically, the cartridge body 41810 comprises a cam slot 41815 defined therein and the tissue cutting knife 41830 comprises a cam pin 41835 extending into the cam slot 41815 that moves within the cam slot 41815 during the staple firing stroke and is moved downwardly by a sidewall 41816 of the cam slot 41815 when the tissue cutting knife 41830 reaches the end of the staple firing stroke. As illustrated in FIG. 174B, the sidewall 41816 of the cam slot 41815 guides the cam pin 41835 into a dwell 41817 that prevents the blade 41832 of the tissue cutting knife 41830 from extending above the deck 41812. The cartridge body 41810 comprises at least one bump or detent that holds the cam pin 41835 in the dwell 41817 so that the tissue cutting knife 41830 cannot move back up into its raised position. Once the cam pin 41835 is in the dwell 41817 and, accordingly, the blade 41832 is recessed below the deck 41812, the firing driver of the surgical stapling instrument can be retracted leaving behind the sled 41820 in its distal fired position and the tissue cutting knife 41830 in its descended position. In such instances, as a result, the knife blade 41832 is not exposed and is unlikely to accidentally come into contact with the patient tissue and/or the clinician when the clinician is removing the staple cartridge from the patient and/or removing the staple cartridge from the surgical stapling instrument.

Further to the above, in at least one instance, the wedge portion 41140 comprises a first material and the knife portion 41150 comprises a second material that is different than the first material. For example, the wedge portion 41140 comprises a plastic material and the knife portion 41150 comprises a metal material. However, in at least one instance, the knife portion 41150 comprises a plastic material and the wedge portion 41140 comprises a metal material.

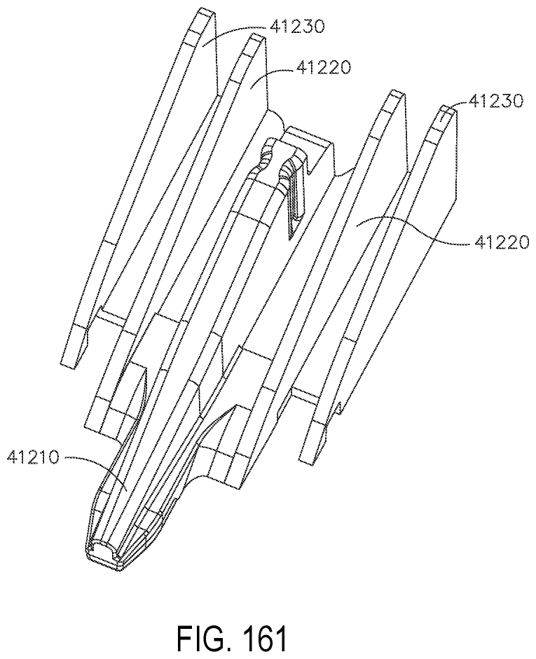

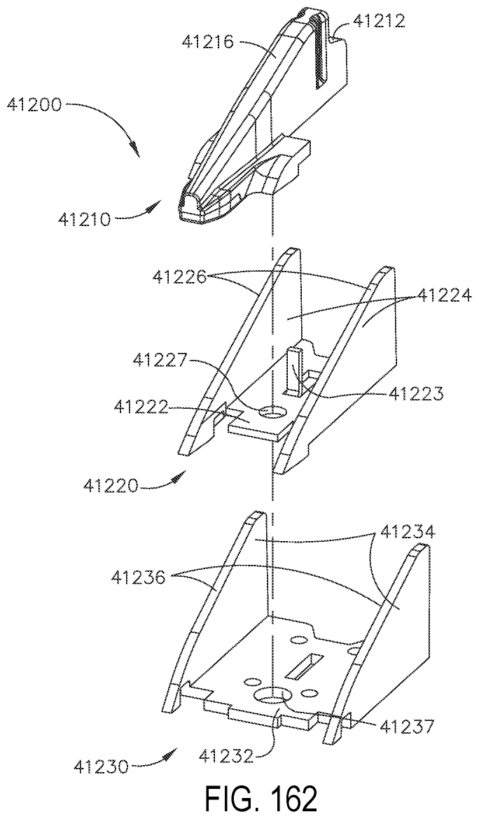

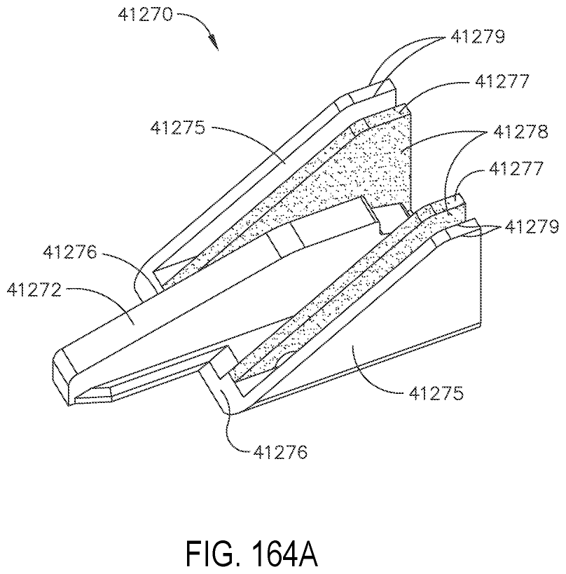

FIGS. 161-164 illustrate a multi-component sled assembly 41200 for use with a staple cartridge such as the staple cartridges described herein, for example. The sled assembly 41200 comprises a central nose component 41210, an inner rail component 41220, and an outer rail component 41230. The central nose component 41210 comprises a proximal ledge 41212 and a distal ramp 41216 configured to move within a longitudinal slot of the staple cartridge. The proximal ledge 41212 is configured to support a distal nose portion of a firing driver to defeat a firing driver lockout, as described herein. The central nose component 41210 further comprises a bottom surface 41211, a cavity 41213 defined in the bottom surface 41211, and a post 41215. In FIG. 164, the post 41215 is unformed and comprises a cylindrical shape, for example, but can comprise any suitable shape. As discussed further below, the post 41215 is heat-staked during a manufacturing process to connect the central nose component 41210, the inner rail component 41220, and the outer rail component 41230.

Further to the above, the inner rail component 41220 comprises an inner base 41222 and inner rails 41224 extending upward from the inner base 41222. Each of the inner rails 41224 comprises an inner distal ramp 41226. The inner rail component 41220 further comprises a central tab 41223 extending upward from the inner base 41222 and a through hole 41227 extending through the base 41222. The outer rail component 41230 comprises an outer base 41232 and outer rails 41234 extending upward from the outer base 41232. Each of the outer rails 41234 comprises an outer distal ramp 41236. The outer base 41232 further comprises a through hole 41237 defined through the outer base 41232.

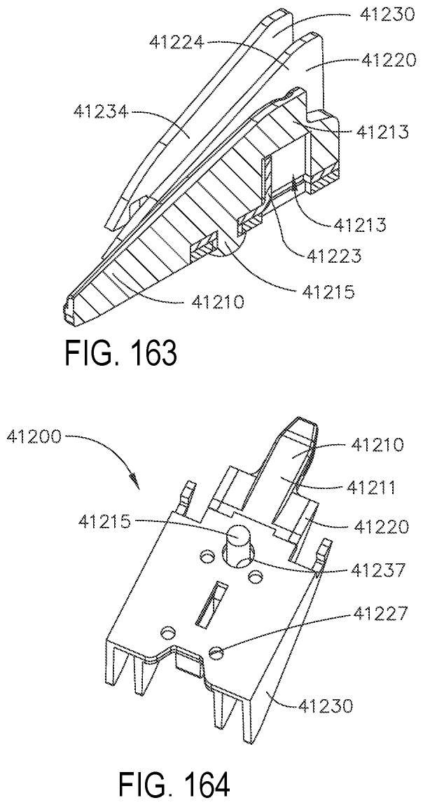

To assemble the sled assembly 41200, further to the above, the inner rail component 41220 is placed onto the outer rail component 41230 such that the through hole 41227 of the inner rail component 41220 aligns with the through hole 41237 of the outer rail component 41230. The central nose component 41210 is then placed onto the inner rail component 41220 such that the central tab 41223 of the inner rail component 41220 is received in the cavity 41213 of the central nose component 41210 to align the inner rail component 41220 with the central nose component 41210. Once assembled, the post 41215 of the central nose component 41210 extends through the through hole 41227 of the inner rail component 41220 and the through hole 41237 of the outer rail component 41230 as illustrated in FIG. 164. In FIG. 164, the post 41215 has not yet been heat-staked and extends beyond the base 41232 of the outer rail portion 41230. The central nose component 41210 is comprised of plastic, and when heat is applied to the post 41215, the post 41215 deforms such that its diameter exceeds that of the aperture 41237 in the outer rail component 41230 and the aperture 41227 in the inner rail component 41220 which locks the central nose component 41210, the inner rail component 41220, and the outer rail component 41230 to one another as illustrated in FIG. 163. In at least one instance, the central tab 41223 of the inner rail component 41220 is press-fit into the cavity 41213 of the central nose component 41210 to aid in coupling the central nose component 41210 to the inner rail component 41220.

In use, as discussed above, the sled assembly 41200 is configured to be advanced through a staple cartridge during a staple firing stroke to cammingly engage and move the staple drivers of the staple cartridge from an unfired position to a fired position to eject staples from the staple cartridge. Specifically, the inner distal ramps 41226 and the outer distal ramps 41236 of the sled assembly 41200 are configured to cammingly engage cam drive surfaces on the staple drivers to lift the staple drivers from their unfired positions to their fired positions as the sled assembly 41200 is advanced distally from a proximal unfired position to a distal fired position by a firing driver. When the firing driver is retracted after the staple firing stroke, the sled assembly 41200 remains in a distal fired position. In other words, the firing driver is configured to advance the sled assembly 41200 but not retract the sled assembly 41200. However, in various embodiments, a firing driver, such as the firing driver 40910, for example, is attachable to the sled assembly 41200 to advance the sled assembly 41200 from its proximal unfired position to a distal fired position and retract the sled assembly 41200 from a distal fired position to its proximal unfired position.

Further to the above, the inner rail component 41220 and the outer rail component 41230 of the sled assembly 41200 are made from stamped metal and the central nose portion 41210 of the sled assembly 41200 is made of plastic. As discussed above, the inner rail component 41220 and the outer rail component 41230 contact and drive the staple drivers of a staple cartridge to eject the staples out of the staple cartridge and drive the staples against an anvil positioned opposite the staple cartridge. In such instances, large forces are transmitted through the rail components 41220, 41230 and, as such, constructing the rail components 41220, 41230 can allow the rail components 41220, 41230 to withstand the large forces without deflecting, or substantially deflecting, as a result thereof. If the rails 41224, 41234, respectively, of the rail components 41220, 41230 were to deflect significantly while driving staples against the anvil, however, the staples being driven against the anvil may not be deformed to the proper formed height. More specifically, the formed height of the deformed staples may be too tall and the deformed staples may not properly clinch the patient tissue therein. That said, the metal rail components 41220, 41230 can be stiff and the interfacing surfaces between the cartridge body and the metal rail components 41220, 41230 can resist the movement of the sled assembly 41200 relative to the cartridge body. With this in mind, the central nose component 41210 of the sled assembly 41200 is comprised of plastic and is configured to slide within the cartridge body of the staple cartridge which is also comprised of plastic. The interfacing plastic surfaces between the central nose component 41210 and the cartridge body may provide little resistance to the movement of the sled assembly 41200 relative to the cartridge body.

As described above, the central nose component 41210 is comprised of plastic. In various embodiments, the central nose component 41210 is over-molded onto the inner rail component 41220 and/or the outer rail component 41230 to form the multi-component sled 41200 into a one-piece, unitary structure. Furthermore, in various embodiments, the central nose component 41210 is heat staked onto the inner rail component 41220 and the outer rail component 41230 to form the multi-component sled 41200 into a one-piece, unitary structure. Furthermore, in at least one embodiment, the inner rails 41224 of the inner rail component 41220 comprise stamped metal and the remainder of the inner rail component is comprised of plastic which has been over-molded and/or heat staked onto the inner rails 41224. Furthermore, in at least one embodiment, the outer rails 41234 of the outer rail component 41230 comprise stamped metal and the remainder of the outer rail component is comprised of plastic which has been over-molded and/or heat staked onto the outer rails 41234.

FIG. 164A illustrates another example of a sled 41270 for use with a staple cartridge, such as the staple cartridges described herein, for example. The sled 41270 comprises a central nose portion 41272, a pair of outer rails 41275, and a pair of inner rails 41278. The inner rails 41278 are comprised of metal while the central portion 41272 and the outer rails 41275 are comprised of plastic. The inner rails 41278 are comprised of stamped stainless steel and the plastic comprising the central nose portion 41272 and the outer rails 41275 has been overmolded onto the inner rails 41278. The outer rails 41275 are not as stiff as the inner rails 41278 and, thus, staples formed by staple drivers driven by the outer rails 41275 may be formed to a larger formed staple height as compared to staples formed by staple drivers driven by the stiffer metal inner rails 41278. In at least one embodiment, a staple cartridge comprises two inner longitudinal row of staple drivers that are driven by the metal inner rails 41278 and two outer longitudinal rows of staple drivers—that are unconnected to the inner longitudinal rows of staple drivers—that are driven by the plastic outer rails 41275. Such an embodiment may experience less resistance during the staple firing stroke than the embodiment depicted in FIG. 13. Moreover, such an embodiment may provide smaller formed staples that tightly clinch the patient tissue adjacent the incision in the patient tissue and larger formed staples that clinch the patient tissue further away from the incision but in a manner that sufficiently permits blood, for example, to flow to the stapled tissue. In at least one embodiment, each staple driver in the inner longitudinal rows of staple drivers is configured to drive two staples while each staple driver in outer longitudinal rows of staple drivers is configured to drive one staple. In such embodiments, the metal sled rails 41278 may drive twice as many staples as the plastic sled rails 41275 during a staple firing stroke and, thus, may experience about twice the firing force therein. Given the metal material that comprises the inner sled rails 41278, the metal sled rails 41278 can endure the higher firing loads without deflecting unacceptably.

In various embodiments, a sled rail can be comprised of metal or plastic. In at least one embodiment, a sled rail is comprised of both metal and plastic. For instance, referring again to FIG. 13A, the inner sled rails 41278 of the sled 41270, for example, are comprised of a metal frame and a plastic material overmolded on a portion of the metal frame. In at least one such embodiment, the bottom 41276 of each inner sled rail 41278 is overmolded with plastic and the top 41377 of each inner sled rail 41278 is not overmolded with plastic. In such embodiments, the plastic bottoms 41276 of the inner sled rails 41278 can provided a smooth surface that initially engages the staple drivers as the sled 41270 is moved distally. As the sled 41270 continues to move distally, the staple drivers move up the inner sled rails 41278 onto the metal surfaces of the inner sled rails 41278. Once the staple drivers reach the tops 41277 of the inner sled rails 41278, the staple drivers have driven the staples to their fully-formed deformed heights. In this position, the inner sled rails 41278 comprise metal columns extending under and supporting the staple drivers. In various embodiments, the metal frames of the inner sled rails 41278 comprise notches defined therein which receive some of the overmolded plastic and interlock the plastic and metal components.

In various embodiments, referring again to FIG. 13A, the top surfaces of the outer sled rails 41275 and the inner sled rails 41278 comprise flat surfaces. As also illustrated in FIG. 13A, each sled rail 41275, 41278 comprises a constant width along the longitudinal length thereof. In other embodiments, one or more of the sled rails 41275, 41278 comprise chamfers and/or fillets on the lateral edges 41279 of the top surfaces which decrease the lateral width of the top surfaces. Such an arrangement can control the profile of the surfaces in contact with the staple drivers.

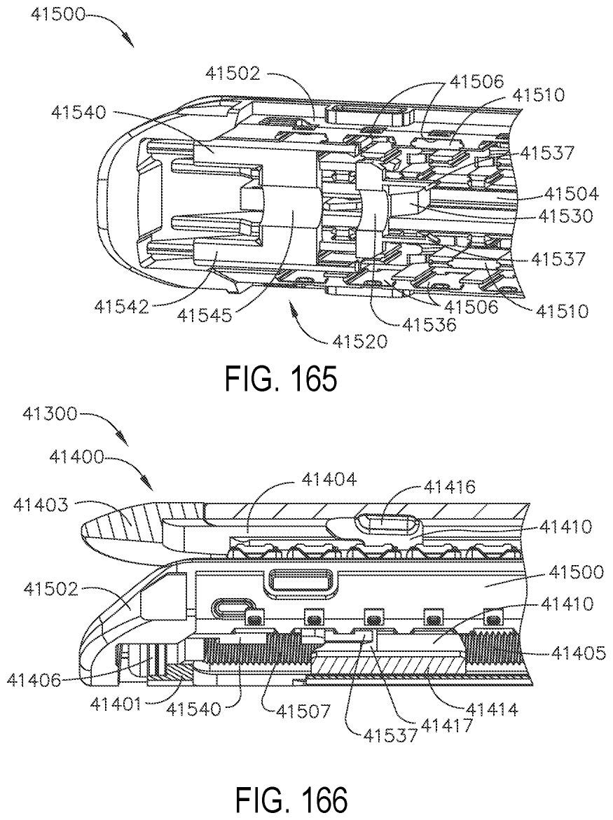

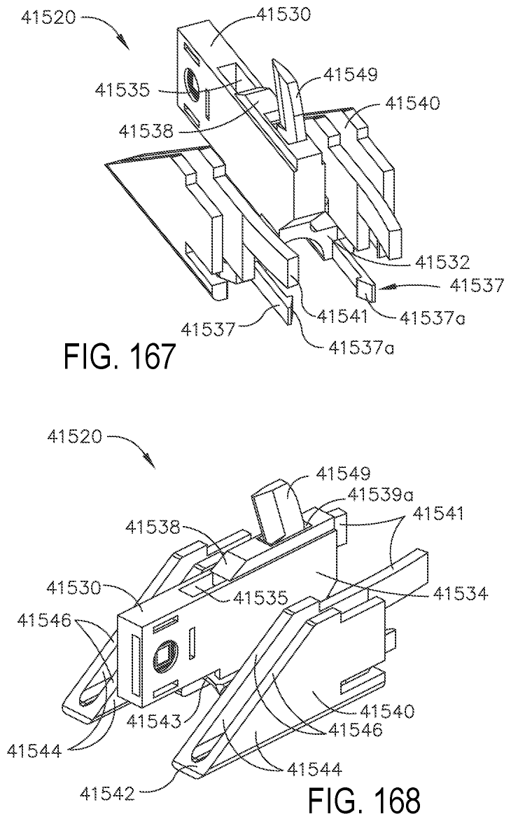

FIGS. 165-170 illustrate a surgical system 41300 comprising an end effector 41400 configured to receive a staple cartridge 41500. The end effector 41400 comprises a first jaw 41401 and a second jaw 41403 movable relative to the first jaw 41401 to capture tissue in between the first jaw 41401 and the second jaw 41403. However, in at least one embodiment, the first jaw 41401 is movable relative to the second jaw 41403. The end effector 41400 further comprises a firing driver 41410 movable relative to the first jaw 41401 and the second jaw 41403. The first jaw 41401 comprises a longitudinal drive screw 41405 rotatably supported at its distal end by a bearing 41406. The firing driver 41410 comprises a firing nut 41412, a first lateral cam 41414, a second lateral cam 41416, and lateral recesses 41417 defined on the lateral sides of the firing driver 41410. The firing nut 41412 comprises an internal thread that is threadingly engaged with external threads 41507 of the drive screw 41405 of the first jaw 41401. When the drive screw 41405 is rotated in a first direction, the firing driver 41410 is pushed distally by the drive screw 41405 and, correspondingly, the drive screw 41405 pulls the firing driver 41410 proximally when the drive screw 41405 is rotated in a second, or opposite direction. The first lateral cam 41414 is configured to cammingly engage the staple cartridge 41500 and/or the first jaw 41401 when the staple cartridge 41500 is positioned in the first jaw 41401 and the firing driver 41410 is moved from a proximal position to a distal position during a firing stroke. The second lateral cam 41416 is configured to move within a longitudinal cavity 41404 of the second jaw 41403 and cammingly engage the second jaw 41403 during the firing stroke. In such instances, the first and second lateral cams 41414, 41416 can approximate, or position, the first jaw 41401 relative to the second jaw 41403 during the firing stroke of the firing driver 41410.

Further to the above, the staple cartridge 41500 comprises a cartridge body 41502 including a longitudinal slot 41504 and a plurality of staple cavities 41506 defined therein. The staple cavities 41506 are arranged in a plurality of longitudinal rows in the cartridge body 41502. Specifically, three rows of staple cavities 41506 are arranged on a first side of the longitudinal slot 41504 and three rows of staple cavities 41506 are arranged on a second side of the longitudinal slot 41504 opposite the first side; however, any suitable arrangement can be utilized. The staple cartridge 41500 further comprises a plurality of staple drivers 41510 movably positioned in the cartridge body 41502. Each of the staple drivers 41510 comprises a staple support portion that moves within a staple cavity 41506 and is configured to support a staple thereon.

Further to the above, the staple cartridge 41500 comprises a multi-component sled 41520 including a nose component 41530 and a wedge component 41540 that are movable relative to one another. The wedge component 41540 comprises a base 41542 and a plurality of rails 41544 extending upward from the base 41542. Specifically, two rails 41544 are positioned on a first side of a central portion 41543 of the base 41542 and two rails 41544 are positioned on a second side of the central portion 41543 opposite the first side. The central portion 41543 of the base 41542 comprises a concave surface 41545 configured to receive a portion of the drive screw 41405—but is not threadably engaged with the drive screw 41405—when the staple cartridge 41500 is positioned in the end effector 41400. Further, each of the rails 41544 comprises a distal ramp 41546 facing distally and configured to cammingly engage the staple drivers 41510 to eject the staples from the staple cartridge 41500. The central portion 41543 comprises an upstanding body 41547 and a knife 41549 pivotably connected to the upstanding body 41547 by a pin 41548. Further, the wedge component 41540 comprises flexible tabs 41541 extending proximally from the inner most rails of the rails 41544, i.e., closest to the central portion 41543. The flexible tabs 41541 are bent inwardly toward each other.

Further to the above, the nose component 41530 of the multi-component sled 41520 comprises a base 41532 and an upstanding body 41534 extending upward from the base 41532. The base 41532 comprises a concave surface 41536 and proximally extending tabs 41537 on either side of the concave surface 41536. Each of the tabs 41537 comprises an inward detent 41537a extending toward one another. The tabs 41537 are configured to latchingly engage with the lateral recesses 41417 of the firing driver 41410, as discussed further below. Further, the concave surface 41536 is configured to receive a portion of the drive screw 41405—but is not threadably engaged with the drive screw 41405—when the staple cartridge 41500 is positioned in the end effector 41400. As such, the wedge component 41540 and the nose component 41530 of the sled 41520 are configured to be seated onto and/or over the drive screw 41405 when the staple cartridge 41500 is positioned in the end effector 41400. After the staple cartridge 41500 is seated in the end effector 41400, the firing driver 41410 can be advanced distally toward the sled 41520 to perform the staple firing stroke. As the firing driver 41410 approaches the sled 41520, the firing driver 41410 abuts proximal surfaces 41531a, 41531b of the nose component 41530 to advance the nose component 41530 distally relative the wedge component 41540.

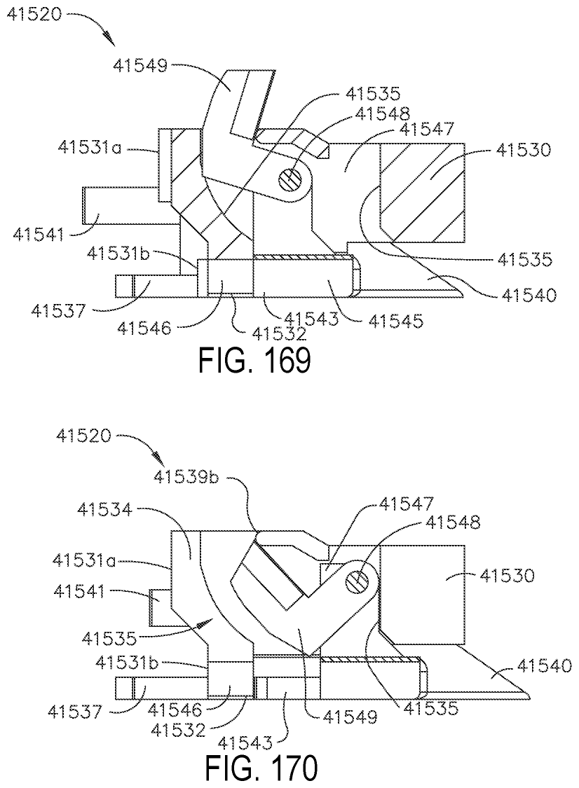

Further to the above, the upstanding body 41534 of the nose component 41530 comprises a cavity 41535 defined therein. The cavity 41535 is configured to receive the upstanding body 41547 of the wedge component 41540 therein. At the outset of the staple firing stroke, a proximal sidewall of the cavity 41535 of the nose component 41530 cammingly engages the knife 41549 of the wedge component 41540 to rotate the knife 41549 from a retracted position (FIG. 170) to an exposed position (FIG. 169). As the firing driver 41410 is advanced further distally, the base 41532 of the nose component 41530 abuts the central portion 41543 of the wedge component 41540 (see FIG. 169) such that the firing driver 41410 advances the nose component 41530 and the wedge component 41540 distally together with the knife 41549 in the exposed position. The knife 41549 of the wedge component 41540 is maintained in the exposed position by the sidewall of the cavity 41535 of the nose component 41530 during the staple firing stroke. During the staple firing stroke, further to the above, the sled 41520 engages the staple drivers 41510 to eject the staples from the staple cartridge 41500. Further, the exposed knife 41549 is configured to incise patient tissue captured in the end effector 41400 during the staple firing stroke of the firing driver 41410.

When the firing driver 41410 abuts the sled 41520 to advance the sled 41520 distally, further to the above, the proximal tabs 41537 of the nose component 41530 of the sled 41520 latchingly engage the lateral recesses 41417 of the firing driver 41410 to operably couple the nose component 41530 of the sled 41520 to the firing driver 41410. After the firing driver 41410 advances the sled 41520 distally to a completely fired, or at least partially fired position, the nose component 41530 is retracted proximally with the firing driver 41410 owing to this coupling. As the nose component 41530 is retracted proximally from the position in FIG. 169, a knife retraction surface 41539b of the nose component 41530 engages and rotates the knife 41549 of the wedge component 41540 from the exposed position (FIG. 169) to the retracted position (FIG. 170). The upstanding body 41534 of the nose component 41530 further comprises a cap 41538 comprising a slot 41539a defined therein. The slot 41539a comprises a distal knife retraction surface 41539b configured to engage and rotate the knife 41549 of the wedge component 41540 as the nose component 41530 is retracted. As discussed below, the wedge component 41540 is prevented from being retracted proximally with the nose component 41530.

As the wedge component 41540 is advanced distally during the staple firing stroke, the flexible tabs 41541 extending proximally from the wedge component 41540 are generally compressed inwardly by the cartridge body 41502. That said, the flexible tabs 41541 are afforded room to resiliently flex outwardly into the staple cavities 41506 as the flexible tabs 41541 pass by the staple cavities 41506 during the staple firing stroke. Such resilient flexing and relaxing of the flexible tabs 41541 during the staple firing stroke does not stop the staple firing stroke; however, in the event that the wedge component 41540 were to be pulled proximally after being advanced through at least a portion of the staple firing stroke, the proximal ends of the flexible tabs 41541 are configured to engage the sidewalls of a staple cavity 41506 and prevent the retraction of the wedge component 41540. As such, the nose component 41530 is retractable proximally with the firing driver 41410, but not the wedge component 41540. When the nose component 41530 is retracted back into its proximal unfired position, the nose component 41530 contacts a stop surface of the cartridge body 41502 which prevents the nose component 41530 from being further retracted proximally. At such point, however, the firing driver 41410 has not yet been returned to its proximal unfired position. When the firing driver 41410 is further retracted with enough force, the proximal tabs 41537 of the nose component 41530 disengage from the lateral recesses 41417 of the firing driver 41410 to decouple the nose component 41530 from the firing driver 41410. The firing driver 41410 can then be retracted back into its original proximal unfired position to permit the spent staple cartridge 41500 to be removed from the end effector 41400. Owing to the recessed position of the knife 41549 in such instances, the possibility of a user coming into contact with the knife 41549 while removing the staple cartridge 41500 from the end effector 41400 is reduced.

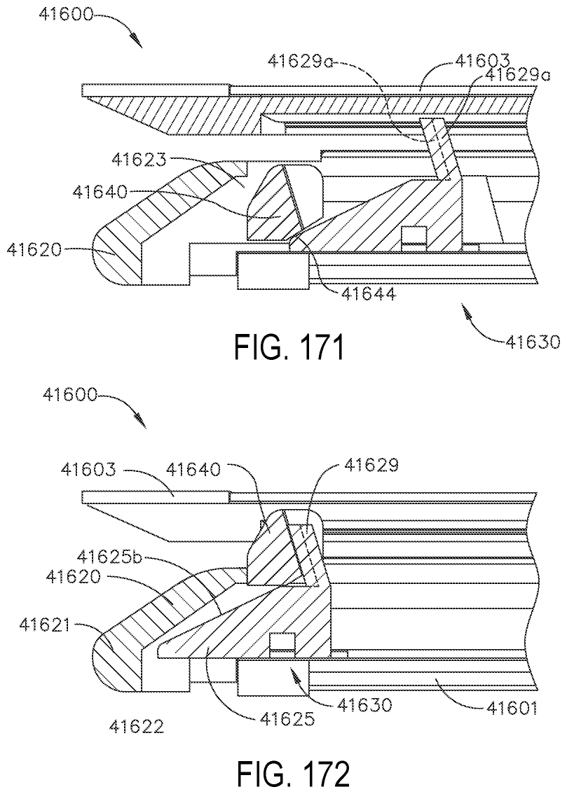

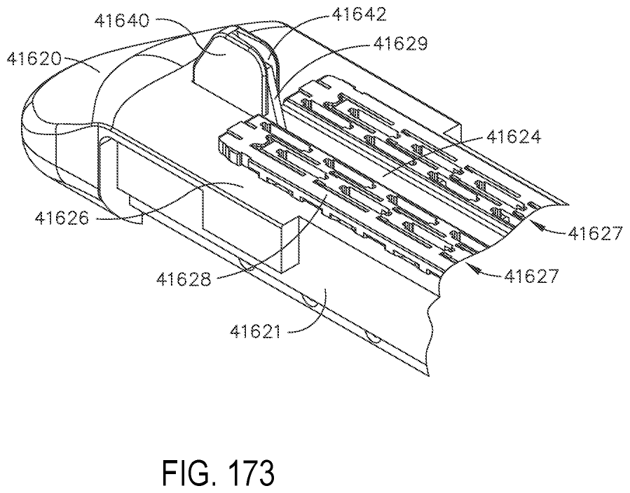

FIGS. 171-173 illustrate a surgical end effector 41600 configured to receive a staple cartridge 41620. The end effector 41600 comprises a first jaw 41601 and a second jaw 41603 movable relative to the first jaw 41601 to capture tissue in between the first jaw 40601 and the second jaw 41603. However, in at least one embodiment, the first jaw 41601 is movable relative to the second jaw 41603. The staple cartridge 41620 comprises a cartridge body 41621 and a sled 41630 movable relative to the cartridge body 41621. The cartridge body 41621 comprises a longitudinal slot 41624, a deck 41626, and a plurality of longitudinal rows of staple cavities 41627 defined in the deck 41626. Three rows of staple cavities 41627 are positioned on a first side of the longitudinal slot 41624 and three rows of staple cavities 41627 are positioned on a second side of the longitudinal slot 41624 opposite the first side. Other arrangements of staple cavities and staples are contemplated. The staple cartridge 41620 further comprise a plurality of staple drivers movably positioned within the staple cavities 41627. The staple drivers are configured to support staples thereon and are similar to the staple drivers 41510 (see FIG. 165), for example.

Further to the above, the sled 41630 of the staple cartridge 41620 is configured to be moved from a proximal unfired position to a distal fired position by a firing driver, such as the firing driver 40910 (FIG. 154), for example, during a firing stroke. The sled 41630 comprises a central nose portion 41625 and a plurality of upstanding rails, such as those described herein, on either side of the central nose portion 41625. Each of the rails comprises a distal ramp configured to cammingly engage the staple drivers of the staple cartridge 41620 to eject the staples from the cartridge body 41621. The central nose portion 41625 comprises a distal ramp 41625b and a knife 41629 extending upwardly from the central nose portion 41622. The central nose portion 41622 moves within the longitudinal slot 41624 of the cartridge body 41621 during the staple firing stroke such that the knife 41629 extends above the cartridge body 41621 to incise patient tissue captured between the first and second jaws 41601, 41603 of the end effector 41600.

Further to the above, the staple cartridge 41620 comprises a movable knife housing 41640 positioned at a distal end 41623 of the cartridge body 41621 within the longitudinal slot 41624. The knife housing 41640 comprises a proximal opening 41642 and a proximal ramp 41644 and slides within a track defined in the cartridge body 41621. The track comprises a vertical slot that extends orthogonally to the longitudinal slot 41624; however, the track can extend in any suitable direction. In use, the knife housing 41640 is movable relative to the cartridge body 41621 between a recessed positon (FIG. 171) and an extended, or exposed, position (FIG. 172). When the sled 41630 is advanced from the proximal unfired position to the distal fired position (FIG. 172), the distal ramp 41625b of the central nose portion 41625 of the sled 41630 cammingly engages the proximal ramp portion 41644 of the knife housing 41640 to lift the knife housing 41640 from the recessed position (FIG. 171) to the exposed position (FIG. 172). When the sled 41630 is in the distal fired position depicted in FIG. 172, at least a portion of the knife 41629 enters the proximal opening 41642 in the knife housing 41640. Specifically, a distal cutting edge 41629a of the knife 41629 is covered by, or enclosed within, the knife housing 41640 when the sled 41630 is in the distal fired position (FIG. 172). When the firing driver is retracted to a proximal position within the end effector 41600, the sled 41630 remains in the distal fired position with the knife housing 41640 covering the distal cutting edge 41629a of the knife 41629. As such, when the spent staple cartridge 41620 is removed from the end effector 41600, the distal cutting edge 41629a of the knife 41629 is not exposed.

Further to the above, in at least one instance, the sled 41630 is prevented from moving proximally and exposing the distal cutting edge 41629a of the knife 41629 after the firing stroke is completed owing to the engagement of the sled 41630 with the cartridge body 41621. More specifically, in various instances the sled 41630 comprises proximal engagement features, such as the flexible tabs 41541 (see FIG. 168), which prevent the sled 41630 from moving, or at least substantially moving, proximally. As such, when the staple cartridge 41620 is removed from the end effector 41600, the distal cutting edge 41629a of the knife 41629 remains covered by the knife housing 41640 and, thus, the possibility of a user coming into contact with the knife 41549 is reduced.

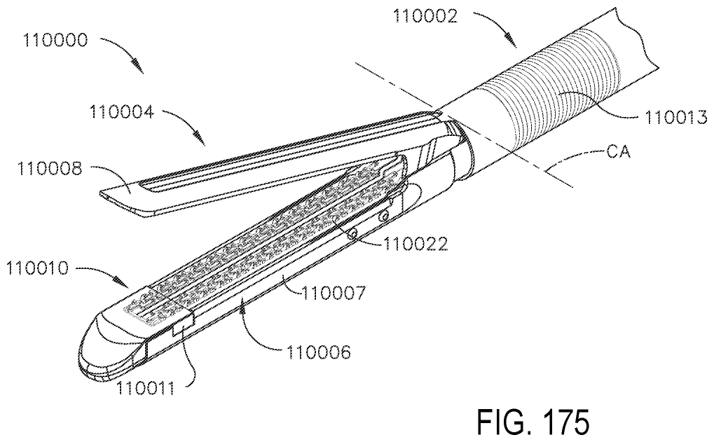

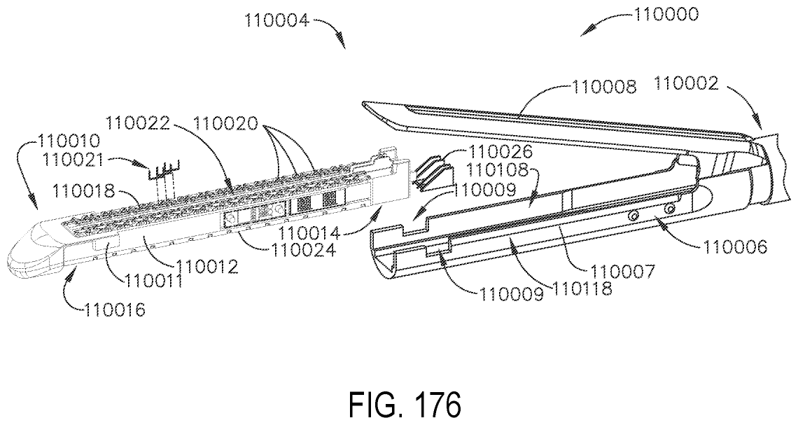

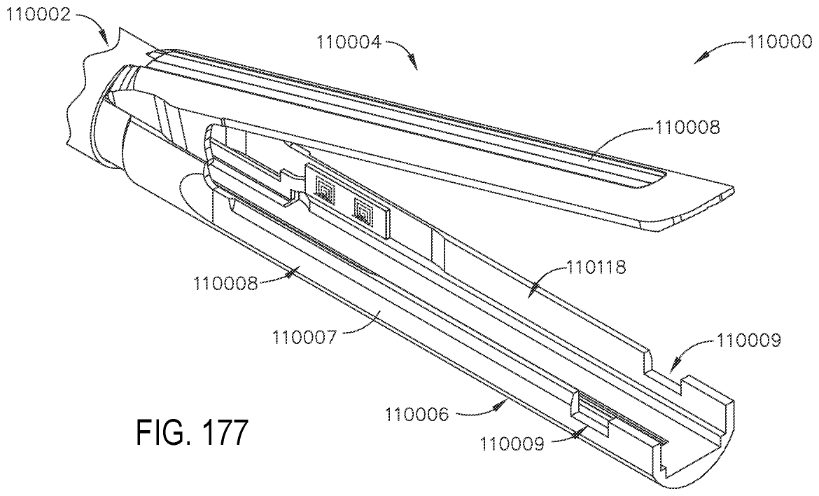

Referring now to FIGS. 175-179, a surgical stapling system 110000 is shown that comprises a shaft 110002 and an end effector 110004 extending from the shaft 110002. The end effector 110004 comprises a first jaw 110006 and a second jaw 110008. The first jaw 110006 comprises an elongate channel 110007 and a staple cartridge 110010. The staple cartridge 110010 is insertable into and removable from the elongate channel 110007. In particular, the staple cartridge 110010 comprises tabs 110011 on each lateral side thereof that snap into corresponding recesses 110009 defined in the elongate channel 110007. Other embodiments are envisioned in which the staple cartridge 110010 is not removable from, or at least readily replaceable from, the first jaw 110006.

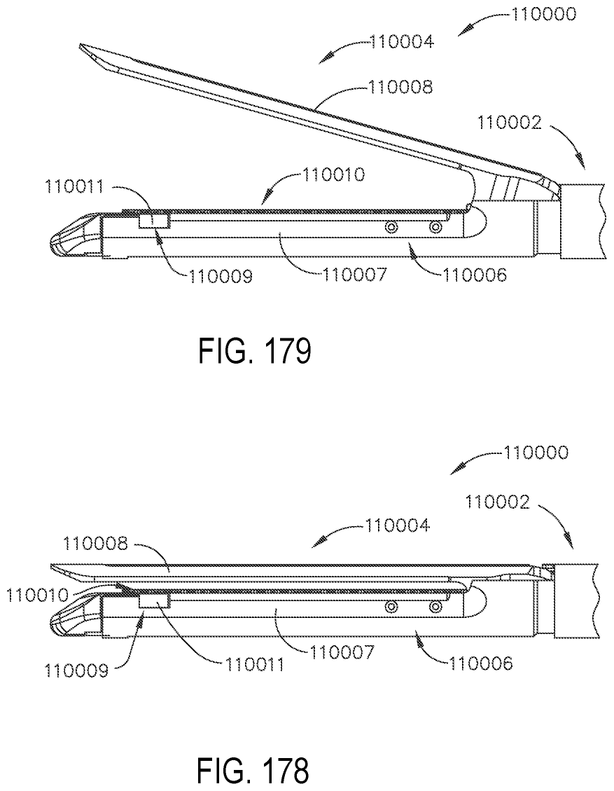

The second jaw 110008 comprises an anvil configured to deform staples ejected from the staple cartridge 110010. The second jaw 110008 is pivotable relative to the first jaw 11006 about a closure axis CA between an open position (FIG. 179) and a closed position (FIG. 178); however, other embodiments are envisioned in which the first jaw 110006 is pivotable relative to the second jaw 110008. The surgical stapling system 110000 further comprises an articulation joint 110013 configured to permit the end effector 110004 to be rotated, or articulated, relative to the shaft 110002. Other embodiments are envisioned which do not include an articulation joint.

The staple cartridge 110010 comprises a cartridge body 110012. The cartridge body 110012 includes a proximal end 110014, a distal end 110016, and a deck 110018 extending between the proximal end 110014 and the distal end 110016. In use, the staple cartridge 110010 is positioned on a first side of the tissue to be stapled and the anvil 110008 is positioned on a second side of the tissue. The anvil 110008 is moved toward the staple cartridge 110010 (see FIGS. 178 and 179) to compress and clamp the tissue against the deck 110018. Thereafter, staples 110021 removably stored in the cartridge body 110012 can be deployed into the tissue. The cartridge body 110012 includes staple cavities 110020 defined therein wherein staples are removably stored in the staple cavities 110020. The staple cavities 110020 are arranged in six longitudinal rows. Three rows of staple cavities are positioned on a first side of a longitudinal slot 110022 and three rows of staple cavities are positioned on a second side of the longitudinal slot 110022. Other arrangements of staple cavities and staples may be possible.

The staples are supported by staple drivers in the cartridge body 110012. Staples supported on staple drivers can be seen in U.S. Patent Application Publication No. 2021/0059672, entitled “SURGICAL INSTRUMENT WITH WIRELESS COMMUNICATION BETWEEN A CONTROL UNIT OF A ROBOTIC SYSTEM AND REMOTE SENSOR”, filed Sep. 14, 2020, which is hereby incorporated by reference in its entirety herein. The drivers are movable between a first, unfired position, and a second, fired, position to eject the staples from the staple cavities 110020. The drivers are retained in the cartridge body 110012 by a retainer 110024 which extends around the bottom of the cartridge body 110012 and includes resilient members configured to grip the cartridge body 110012 and hold the retainer 110024 to the cartridge body 110012. The drivers are movable between their unfired positions and their fired positions by a sled 110026. The sled 110026 is movable between a proximal position adjacent the proximal end 110014 and a distal position adjacent the distal end 110016. The sled 110026 comprises a plurality of ramped surfaces configured to slide under the drivers and lift the drivers, and the staples supported thereon, toward the anvil 110008. In various embodiments, the staples are not supported by staple drivers, but rather, the staples include integral drive surfaces that are directly engaged by the sled 110026 to lift the staples, examples of which are described in U.S. Patent Application Publication No. 2015/0173756, entitled “SURGICAL CUTTING AND STAPLING METHODS”, filed Dec. 23, 2013, which is hereby incorporated by reference in its entirety herein.

Further to the above, the sled 110026 is moved distally by a firing member or firing actuator, such as those seen and described elsewhere herein. The firing actuator is configured to contact the sled 110026 and push the sled 110026 toward the distal end 110016. The longitudinal slot 110022 defined in the cartridge body 110012 is configured to receive the firing actuator. The anvil 110008 also includes a slot defined therein that receives the firing actuator. The firing actuator further comprises a first cam which engages the first jaw 110006 and a second cam which engages the second jaw 110008 in the slot of the anvil. As the firing actuator is advanced distally, the first cam and the second cam can control the distance, or tissue gap, between the deck 110018 of the staple cartridge 110010 and the anvil 110008. The firing actuator also comprises a knife configured to incise the tissue captured intermediate the staple cartridge 110010 and the anvil 110008. It is desirable for the knife to be positioned at least partially proximal to the ramped surfaces such that the staples are ejected ahead of the knife.

In various embodiments, the anvil 110008 is moved from the open position to the closed position using a closure actuator that is controlled separately from the firing actuator. In some such embodiments, the firing actuator is considered to be a part of a firing system that is separate and distinctly operable from the closure actuator. In some such embodiments, the anvil 110008 comprises a ramp on a proximal end thereof and the closure actuator comprises a closure tube that movable distally to engage the ramp and cam the anvil 110008 to the closed position. In the closed position, the first cam and the second cam of the firing actuator translate distally and maintain the anvil 110008 in the closed position. In such arrangements, to transition the anvil 110008 to the open position, the closure tube is retracted proximal and springs positioned within the end effector 110004 bias the anvil 110008 to the open position. In various other embodiments, the anvil 110008 includes a tab and the closure tube defines an aperture which engages the tab as the closure tube moves proximally, thereby positively transitioning the anvil 110008 to the open position. Exemplary closure actuators and closure tubes are described in U.S. Patent Application Publication No. 2021/0059672, entitled “SURGICAL INSTRUMENT WITH WIRELESS COMMUNICATION BETWEEN A CONTROL UNIT OF A ROBOTIC SYSTEM AND REMOTE SENSOR”, filed Sep. 14, 2020, which is hereby incorporated by reference in its entirety herein. Various other closure actuators are contemplated by the present disclosure and are discussed elsewhere herein.

In various other embodiments, the anvil 110008 is moved from the open position to the closed position using the firing actuator. In some such embodiments, the anvil 110008 includes a ramp that extends proximally from walls that define the slot that is engaged by the firing actuator as it moves from a first, proximal to position to a second, intermediate position during a first portion of the stroke of the firing actuator to move the anvil 110008 to the closed position. In some embodiments, the first portion of the stroke of the firing actuator can be about 0.110″-0.250″ of the firing actuator travel. At the end of the first portion of the stroke, the firing actuator can continue advancing distally from the second, intermediate position toward a third, distal position during a second portion of the stroke to deploy staples from the staple cartridge 110010 and incise tissue captured by the end effector, as described above. Exemplary firing actuators that close the anvil and fire staples are described in U.S. Pat. No. 11,160,551, entitled “ARTICULATABLE SURGICAL STAPLING INSTRUMENTS”, filed Dec. 21, 2016, which is hereby incorporated by reference in its entirety herein. In one aspect, when a firing actuator is utilized to move the anvil 110008 toward the closed position and deploy staples from the staple cartridge 110010, the firing actuator can be considered both a closure actuator and/or a firing actuator.

After a staple cartridge 110010 has been fired, or at least partially fired, it is removed from the elongate channel 110007 and then replaced with another replaceable staple cartridge, if desired. At such point, the surgical stapling system 110000 can be re-used to continue stapling and incising the patient tissue. In some instances, however, a previously-fired staple cartridge can be accidentally loaded into the elongate channel 110007. If the firing actuator were to be advanced distally within such a previously-fired staple cartridge, the surgical stapling system 110000 could cut the patient tissue without stapling it. The surgical stapling system 110000 could similarly cut the patient tissue without stapling it if the firing actuator were advanced distally through a staple firing stroke without a staple cartridge positioned in the elongate channel 110007 at all. In addition, various surgical staple cartridges may have different arrays of and/or orientations of staples/fasteners therein. The sizes of the staples or fasteners, as well as the number of fasteners may vary from cartridge type to cartridge type depending upon a particular surgical procedure or application. To ensure that the staples are properly crimped or formed, the surgical staple cartridges must be used in connection with corresponding, compatible anvils that have the proper array of staple-forming pockets therein as well as the proper cutting and firing components. Should a “non-compatible” cartridge be loaded into a surgical stapling device that has an anvil that is mismatched to the staple cartridge, the staples may not be properly formed during the firing process which could lead to catastrophic results.

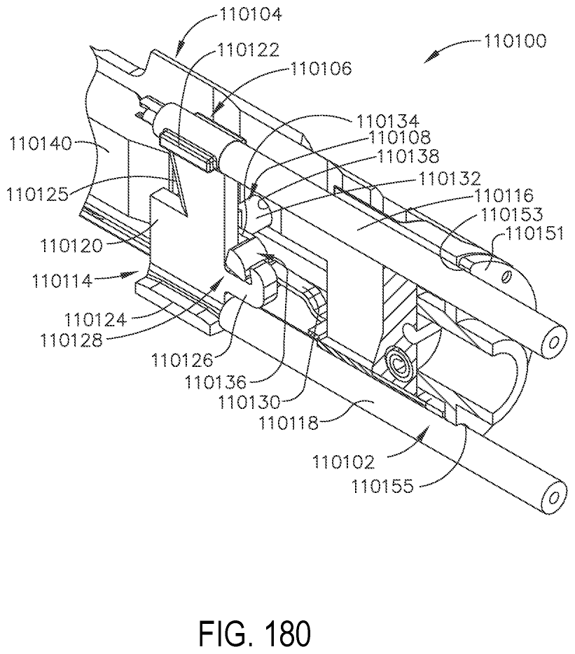

Referring now to FIGS. 180-182, a surgical stapling system 110100 is provided, according to at least one aspect of the present disclosure. The surgical stapling system 110100 comprises a shaft 110102, an end effector 110104, a firing actuator 110106, and a lockout 110108. In some embodiments, the shaft 110102 is similar to shaft 110002. In some embodiments, the end effector 110104 is similar to end effector 110004.

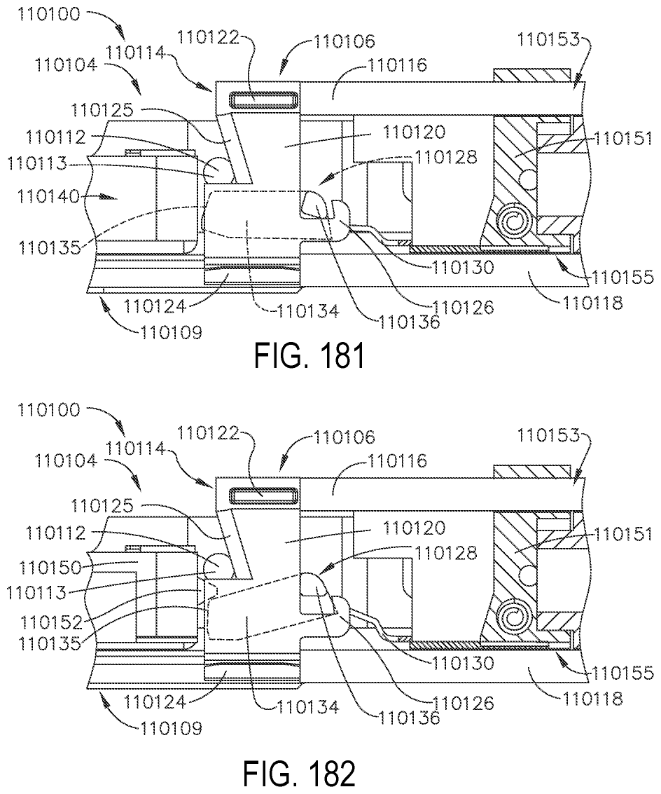

The end effector 110104 comprises an elongate channel 110109, an anvil (not shown, but similar to anvil 110008), and a pivot 110112 pivotably coupling the anvil to the elongate channel 110109 such that the anvil can rotate relative to the elongate channel 110109 between an open position (similar to FIG. 179) and a closed position (similar to FIG. 178). As can be seen in FIGS. 181 and 182, the pivot 110112 comprises a rod 110113 that defines a closure axis (similar to closure axis CA shown in FIG. 175).

The firing actuator 110106 comprises a firing driver 110114, a first push rod 110116 attached at an upper end of the firing driver 110114, and a second push rod 110118 attached at a lower end of the firing driver 110114. The first push rod 110116 and the second push rod 110118 can be driven using any suitable drive system, such as a motor drive system, to drive the firing driver 110114 from a proximal position, shown in FIGS. 181 and 182, to a distal position. An exemplary motor drive system is described in U.S. Patent Application Publication No. 2015/0173756, entitled “SURGICAL CUTTING AND STAPLING METHODS”, filed Dec. 23, 2013, which is hereby incorporated by reference in its entirety herein. The firing actuator comprises a body 110120, a first cam 110122, a second cam 110124, a knife 110125, and a hook 110126, described in more detail below.

In operation, the firing driver 110114 is movable through a stroke that includes a closure stroke portion and a firing stroke portion distal and subsequent to the closure stroke portion. During the closure stroke portion, the first cam 110122 of the firing driver 110114 engages a ramp on the anvil to cam the anvil from the open position toward the closed position. During the firing stroke portion subsequent and distal to the closure stroke portion, the body 110120 of the firing driver 110114 engages and drives a sled (similar to sled 110026) through a staple cartridge to deploy staples therefrom while the knife 110125 cuts the stapled tissue. Furthermore, as the firing driver 110114 is advanced distally through the firing stroke portion, the second cam 110124 engages the elongate channel 110109 and the first cam 110122 engages the anvil to control the distance, or tissue gap, therebetween. In one aspect, since the firing actuator 110106 is utilized to move the anvil toward the closed position prior to deploying staples from a staple cartridge, the firing actuator can also be considered a closure actuator.

The surgical stapling system 110100 further comprises a flange 110151 that defines a first channel 110153 that receives and guides the first push rod 110116 and a second channel 110155 that receives and guides the second push rod 110118 during the stroke of the firing actuator.

The lockout 110108 includes lockout bar 110128 and a spring 110130 coupled to the lockout bar 110128. The lockout bar 110128 includes a pin 110132, a body 110134 defining a cam surface 110135 on its distal end thereof, and a tab 110136. The shaft 110102 defines an aperture 110138 that receives the pin 110132 of the lockout bar 110128. See FIG. 182. In various other embodiments, the elongate channel 110109 defines the aperture 110138 that receives the pin 110132 of the lockout bar 110128. The lockout bar 110128 is pivotable between a locked state in which the tab 110136 is received within the hook 110126 (FIGS. 180 and 181) and an unlocked state in which the tab 110136 is rotated out of, or displaced from, the hook 110126 (FIG. 182). The spring 110130 is mounted to the shaft 110102 and biases lockout bar 110128 toward the locked state. In various other embodiments, the spring 110130 is mounted to the base of the elongate channel 110109. In some embodiments, the spring 110130 comprises a leaf spring. In some embodiments, the spring comprises a torsion spring.

In the locked state, the tab 110136 and the hook 110126 are longitudinally aligned such that, should a user attempt to drive the firing driver 110114 through its stroke portions, the tab 110136 would engage the hook 110126, preventing distal motion of the firing driver 110114. Stated another way, in the locked state, the lockout 110108 prevents the firing driver 110114 from moving through the closure stroke portion and the firing stroke portion of its stroke. Notably, the tab 110136 engages the hook 110126 at a location that is proximal to the pivot 110112, as will be explained in more detail below.

In the unlocked state, the tab 110136 is rotated out of, or displaced from, the hook 110126 such that the tab 110136 and hook 110126 are not longitudinally aligned. Stated another way, in the unlocked state, the lockout 110108 allows the firing driver 110114 to move through the closure stroke portion and the firing stroke portion of its stroke. In one aspect, since the lockout 110108 prevents the firing driver 110114 from moving through the closure stroke portion and the firing stroke portion when it is in the locked state, the lockout 110108 can be considered a closure lockout and/or a firing lockout.

In various embodiments, the majority, if not all, of the lockout 110108 is positioned proximal to the pivot 110112. In the illustrated arrangement, a majority of the lockout bar 110128 is located proximal to the pivot 110112. For example, the cam surface 110135 on the distal end of the lockout bar 110128 may not be proximal to the pivot 110112, however, the point of contact between the firing actuator 110106 (the hook 110126) and the lockout 110108 (the tab 110136) is proximal with respect to the pivot 110112, as described above. By moving the lockout 110108 proximal of the pivot 110112, space is freed up within the end effector 110104 and the lockout 110108 is moved away from the pinch point between the elongate channel 110109 and the anvil. In other embodiments, the entirety of the lockout 110108, such as the cam surface 110135, is positioned proximal to the pivot 110112, freeing up additional space within the end effector 110104.

To transition the lockout 110108 from the locked state to the unlocked state, a compatible staple cartridge is positioned in the elongate channel 110109. Referring to FIGS. 180 and 181, an incompatible staple cartridge 110140 is removably positioned in the elongate channel 110109. Accordingly, the lockout 110108 remains in the locked state, preventing the firing driver 110114 from moving through the closure stroke portion and the firing stroke portion of its stroke. Notably, the incompatible staple cartridge 110140 does not include a key to transition the lockout 110108 to the unlocked state, as described in more detail below. Accordingly, should a user attempt to drive the firing actuator 110106 through its strokes, the tab 110136 would engage the hook 110126 proximal of the pivot 110112, preventing the firing actuator 110106 from moving distally.

Referring now to FIG. 182, a compatible staple cartridge 110150 is removably positioned in the elongate channel 110109. The compatible staple cartridge 110150 includes a key 110152 extending proximally therefrom. As the staple cartridge 110150 is removably positioned in the elongate channel 110109, the key 110152 engages the cam surface 110135 of the lockout bar 110128. As the staple cartridge 110150 is moved proximally to seat the staple cartridge 110150 in the elongate channel 110109, the key 110152 engages the cam surface 110135 and rotates the tab 110136 out of longitudinal alignment with the hook 110126 of the firing driver 110114, thus “defeating” the lockout 110108 and placing the lockout bar 110128 in the unlocked state. Notably, the key 110152 comprises a length that allows the key 110152 to extend proximally from the staple cartridge 110150 to engage the cam surface 110135. As shown in FIG. 182, the point of contact between the key 110152 and the cam surface 110135 is at, or adjacent to, the pivot 110112. In embodiments wherein the entirety of the lockout 110108 is positioned proximal to the pivot, as described above, the key 110152 comprises a length that allows the key 110152 to extend proximally from the staple cartridge 110150, past the pivot 110112, to engage the cam surface 110135 at a location proximal to the pivot 110112.

In some embodiments, the staple cartridge 110150 comprises a cartridge body that comprises the key 110152. In some embodiments, the staple cartridge 110150 comprises a retainer that comprises the key 110152. In some embodiments, the staple cartridge 110150 comprises a sled that comprises the key 110152. In some such embodiments where the sled comprises the key, upon transitioning the lockout bar 110128 to the unlocked state, the firing driver 110114 is permitted to distally advance through the closure stroke portion and the firing stroke portion. As the firing driver 110114 advances distally, the sled is also advanced distally by the firing driver 110114, causing the key 110152 to progressively disengage the cam surface 110135 of the lockout bar 110128. As the key 110152 progressively disengages the cam surface 110135, the spring 110130 progressively biases the lockout bar 110128 toward the locked state. Notably, the hook 110126 of the firing driver 110114 proximally extends a length from the body 110120 that allows the firing driver 110114 to distally clear the tab 110136 before the spring 110130 repositions the lockout bar 110128 into the locked state.

Upon completion of the firing stroke, the firing driver 110114 is retracted toward its proximal position. As the firing driver 110114 is retracted, the proximal end of the hook 110126 engages the distal end of the tab 110136, which cams the lockout bar 110128 upwardly toward the unlocked state, allowing the hook 110126 to slide under the tab 110136. Once the hook 110126 proximally clears the tab 110136, the spring 110130 biases the lockout bar 110128 back into its locked state, preventing advancement of the firing driver 110114 through another closure stroke and firing stroke until another compatible staple cartridge is positioned in the end effector 110104.

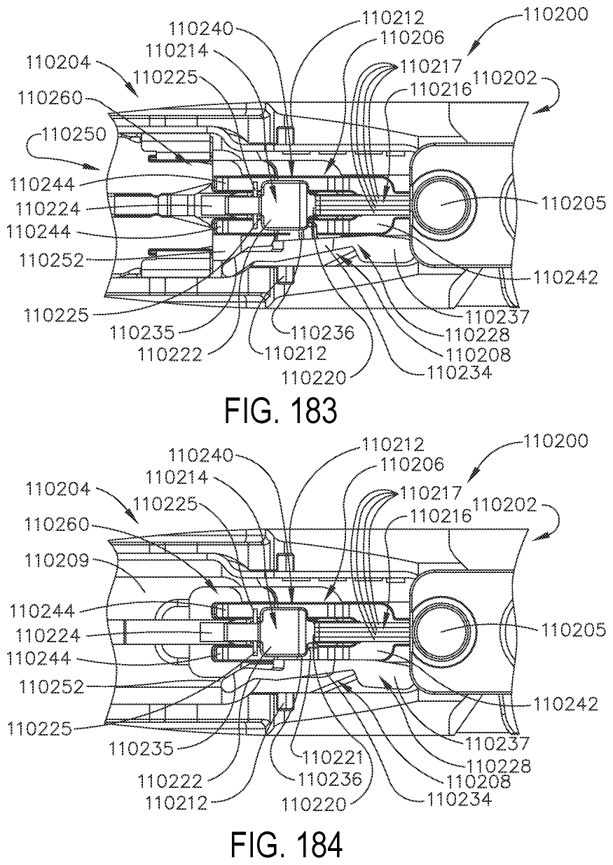

Referring now to FIGS. 183-185, a surgical stapling system 110200 is provided, according to at least one aspect of the present disclosure. The surgical stapling system 110200 comprises a shaft 110202, an end effector 110204, an articulation joint 110205, a firing actuator 110206, a closure actuator 110207, and a lockout 110208. In some embodiments, the shaft 110202 is similar to shaft 110002. In some embodiments, the end effector 110204 is similar to end effector 110004.

The end effector 110204 comprises an elongate channel 110209, an anvil 110210, and a pivot 110212 pivotably coupling the anvil 110210 to the elongate channel 110209 such that the anvil 110210 can rotate relative to the elongate channel 110209 between an open position (similar to FIG. 179) and a closed position (similar to FIG. 178). In at least one arrangement, pivot 110212 comprises a pin. The end effector 110204 is pivotable relative to the shaft 110202 about the articulation joint 110205. The anvil 110210 comprises a tab 110215 extending from a proximal end thereof, as will be discussed in more detail below.

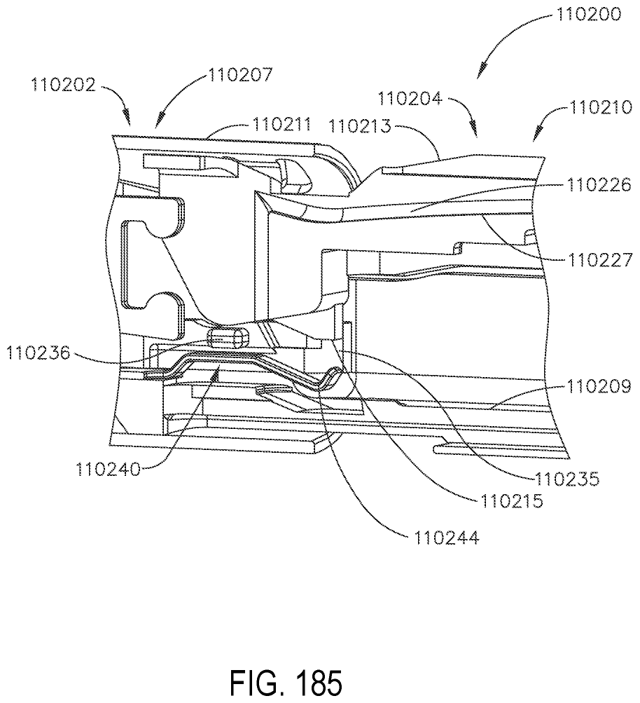

The closure actuator 110207 comprises a closure tube 110211 that can be driven using any suitable drive system, such as a motor drive system or manual drive system, to drive the closure tube 110211 from a proximal position, shown in FIG. 185, to a distal position. The anvil 110210 comprises a cam surface or ramp 110213 defined on a proximal end thereof. As the closure tube 110211 moves distally, the closure tube 110211 contacts the cam surface 110213 to cam, or rotate, the anvil 110210 from the open position toward the closed position. In some embodiments, to transition the anvil 110210 to the open position, the closure tube 110211 is retracted proximally and springs positioned within the end effector 110204 bias the anvil 110210 to the open position. In various other embodiments, the anvil 110210 includes a tab and the closure tube 110211 defines an aperture which engages the tab as the closure tube 110211 moves proximally to positively transition the anvil 110210 to the open position. Exemplary closure actuators and closure tubes are described in U.S. Patent Application Publication No. 2021/0059672, entitled “SURGICAL INSTRUMENT WITH WIRELESS COMMUNICATION BETWEEN A CONTROL UNIT OF A ROBOTIC SYSTEM AND REMOTE SENSOR”, filed Sep. 14, 2020, which is hereby incorporated by reference in its entirety herein. Various other closure actuators are contemplated by the present disclosure and are discussed elsewhere herein.

The firing actuator 110206 comprises a firing member or firing driver 110214 and a firing bar 110216 extending proximally from the firing driver 110214. The firing bar 110216 comprises a plurality of laminated strips 110217. The firing bar 110216 can be driven using any suitable drive system, such as a motor drive system, to drive the firing driver 110214 from a proximal position, shown in FIGS. 183 and 184, to a distal position. An exemplary motor drive system is described in U.S. Patent Application Publication No. 2015/0173756, entitled “SURGICAL CUTTING AND STAPLING METHODS”, filed Dec. 23, 2013, which is hereby incorporated by reference in its entirety herein. The firing driver 110214 comprises a firing actuator body 110220 defining an aperture 110221, a first cam 110222, a second cam (not shown, but similar to second cam 110124), a knife (not shown, but similar to knife 110125), and a lockout body 110224 pivotably coupled to the firing actuator body 110220. The lockout body 110224 comprises pins 110225 extending laterally from each side thereof.

In operation, the closure tube 110211 is moved through a closure stroke to transition the anvil 110210 from the open position to the closed position. Once the anvil 110210 has been placed in the closed position, the firing driver 110214 is moved through a firing stroke. During the firing stroke, the body 110220 of the firing driver 110114 engages and drives a sled through a staple cartridge to deploy staples therefrom while the knife cuts the stapled tissue. Furthermore, as the firing driver 110214 is advanced distally through the firing stroke, the second cam engages the elongate channel 110209 and the first cam 110222 traverses a slot 110226 defined in the anvil 110210 and engages a lower surface 110227 of the slot 110226 to control the distance, or tissue gap, between the elongate channel 110209 and the anvil 110210. In other embodiments, the surgical stapling system 110200 does not include the closure actuator 110207. In some such embodiments, the firing actuator 110206 is configured to advance through a closure stroke portion and a firing stroke portion distal and subsequent to the closure stroke portion. During the closure stroke portion, the firing driver 110214 engages a ramp on a proximal end of the anvil 110210 to move the anvil from the open position toward the closed position. During the firing stroke portion, similar to above, the firing driver 110114 engages and drives a sled through a staple cartridge to deploy staples therefrom while also controlling the distance, or tissue gap, between the elongate channel 110209 and the anvil 110210.

The lockout 110208 includes lockout bar 110228 that includes a body 110234 defining a cam surface 110235 on its distal end thereof and a tab 110236. The lockout bar 110228 is pivotable between a locked state in which the tab 110236 is received within the aperture 110221 of the firing driver 110214 (FIG. 184) and an unlocked state in which the tab 110236 is rotated laterally out of, or displaced from, the aperture 110221 of the firing driver 110214 (FIG. 183). In some embodiments, a proximal end 110237 of the body 110234 is pivotably coupled to the shaft 110202. In some embodiments, the proximal end 110237 of the body 110234 is pivotably coupled to the elongate channel 110209. In some embodiments, the lockout 110208 further comprises a spring, such as a torsion spring, as an example, that biases the lockout bar 110228 toward the locked state.

In the locked state, as referenced above, the tab 110236 is received within the aperture 110221 of the firing driver 110214 such that, should a user attempt to drive the firing driver 110214 through its firing stroke, the tab 110236 would engage a proximal wall defined by the aperture 110221 and prevent distal motion of the firing driver 110214. Stated another way, in the locked state, the lockout 110208 prevents the firing driver 110214 from moving through the firing stroke. Notably, the tab 110236 engages the proximal wall defined by the aperture 110221 at a location that is laterally proximal to the pivot 110212, as will be explained in more detail below.

Furthermore, in the locked state, the body 110234 of the lockout bar 110228 is vertically aligned with the tab 110215 defined on the proximal end of the anvil 110210 such that, should a user attempt to rotate the anvil 110210 toward the closed position, the tab 110215 would engage the body 110234 of the lockout bar 110228 and prevent rotational motion thereof. Stated another way, in the locked state, the lockout bar 110228 prevents the anvil 110210 from moving to the closed position. Accordingly, in the locked state, the lockout 110208 functions to prevent both closure of the anvil 110210 and firing of the firing driver 110214. Therefore, in one aspect, the lockout 110208 can be considered a closure lockout and/or a firing lockout.

In the unlocked state, as referenced above, the tab 110236 is rotated laterally out of, or displaced from, the aperture 110221 of the firing driver 110214. In the unlocked state, the body 110234 of the lockout bar 110228 is also laterally displaced such that it is no longer vertically aligned with the tab 110215 defined on the anvil 110210 and, therefore, should a user attempt to rotate the anvil 110210 toward the closed position, the tab 110215 would bypass the body 110234 of the lockout bar 110228 during rotation of the anvil 110210. Stated another way, in the unlocked state, the lockout bar 110228 permits the anvil 110210 to move to the closed position. Furthermore, in the unlocked state, should a user attempt to drive the firing driver 110214 through the firing stroke, the tab 110236 would not engage the proximal wall defined by the aperture 110221 and prevent distal motion of the firing driver 110214. That is, in the unlocked state, the lockout 110208 permits the firing driver 110214 to move through the firing stroke.

In various embodiments, the majority of, if not all, of the lockout 110208 is positioned proximal to the pivot 110212. In particular, the point of contact between the lockout 110208 (the tab 110236) and the firing driver 110214 (the aperture 110221) is proximal with respect to the pivot 110212. By moving the lockout 110208 proximal of the pivot 110212, space is freed up within the end effector 110204 and the lockout 110208 is moved away from the pinch point between the elongate channel 110209 and the anvil 110210. In other embodiments, the entirety of the lockout 110108 is positioned proximal to the pivot 110212, freeing up additional space within the end effector 110204.

To transition the lockout 110208 from the locked state to the unlocked state, a compatible staple cartridge is positioned in the elongate channel 110209. Referring to FIG. 184, no staple cartridge is removably positioned in the elongate channel 110209. Accordingly, the lockout 110208 remains in the locked state, preventing the anvil 110210 from rotating to the closed position and preventing firing driver 110214 from moving through the firing stroke. Accordingly, should a user attempt to drive the firing driver 110214 through the firing stroke, the tab 110236 would engage the proximal wall defined by the aperture 110221 at a location proximal to the pivot 110212, preventing the firing driver 110214 from moving distally. It should be understood that, should an incompatible staple cartridge that does not include key, discussed in more detail below, be removably positioned in the elongate channel 110209, the lockout 110208 would also remain in the unlocked state.

Referring now to FIG. 183, a compatible staple cartridge 110250 is removably positioned in the elongate channel 110209. The compatible staple cartridge 110250 includes a first key 110252 extending proximally therefrom. As the staple cartridge 110250 is removably positioned in the elongate channel 110209, the first key 110252 engages the cam surface 110235 of the lockout bar 110228. As the staple cartridge 110250 is moved proximally to seat the staple cartridge 110250 in the elongate channel 110209, the first key 110252 engages the cam surface 110235 and laterally rotates the tab 110236 out of the aperture 110221 and the body 110234 out of vertical alignment with the tab 110215, thus “defeating” the lockout 110208 and placing the lockout bar 110228 in the unlocked state. As shown in FIG. 184, the point of contact between the key 110252 and the cam surface 110235 is at a location distal with respect to the pivot 110212. In embodiments where the entirety of the lockout 110208 is positioned proximal to the pivot 110212, the key 110252 comprises a length that allows the key 110252 to extend proximally from the staple cartridge 110250, past the pivot 110212, to engage the cam surface 110235 at a location proximal to the pivot 110212.

In some embodiments, the staple cartridge 110250 comprises a cartridge body that comprises the first key 110252. In some embodiments, the staple cartridge 110250 comprises a retainer that comprises the first key 110252. In some embodiments, the staple cartridge 110250 comprises a sled that comprises the first key 110252. In one aspect, lockout 110208 can be considered a compatible staple cartridge lockout as a compatible staple cartridge (i.e., a cartridge including the first key 110252) is required in order to transition the lockout bar 110228 to the unlocked state. An absent first key 110252 from a staple cartridge can be indicative of the staple cartridge being an incompatible staple cartridge.

As referenced above, the firing driver 110214 comprises a firing actuator body 110220 and a lockout body 110224 pivotably coupled to the firing actuator body 110220. The lockout body 110224 is pivotable relative to the firing actuator body 110220 between an upward, unlocked position and a downward, locked position. In various embodiments, the firing driver 110214 further comprises a spring that biases the lockout body 110224 toward the locked position. The end effector 110204 further comprises a spring 110240 that includes a body 110242 and a pair of hooks 110244 extending distally from the body 110242. In some embodiments, a proximal end of the body 110242 is mounted to the shaft 110202. In some embodiments, the proximal end of the body 110242 is mounted to the elongate channel 110209.

In the locked position of the lockout body 110224, the hooks 110244 of the spring 110240 “catch” the pins 110225 to prevent distal motion of the firing driver 110214. Stated another way, in the locked position of the lockout body 110224, the spring 110240 prevents the firing driver 110214 from moving through its firing stroke. In the unlocked position of the lockout body 110224, the pins 110225 are rotated upward and away from the hooks 110244 such that the lockout body 110224 and the firing driver 110214 are able to traverse past the hooks 110244 without being “caught”. Stated another way, in the unlocked position, the spring 110240 permits the firing driver 110214 to move through its firing stroke. Accordingly, in one aspect, the spring 110240 and the lockout body 110224 can be considered a second lockout, collectively referred to as lockout 110260. In one aspect, the lockout 110260 is considered a firing lockout. In one aspect, the lockout 110260 is considered a ready-to-fire lockout, as will be discussed in more detail below.

To transition the lockout body 110224 from the locked position to the unlocked position, a “ready-to-fire” staple cartridge is positioned in the elongate channel 110209. Referring now to FIG. 183, a compatible and ready-to-fire staple cartridge 110250 is removably positioned in the elongate channel 110209. As discussed above, the compatible staple cartridge 110250 includes a first key 110252 that “defeats” the first lockout 110208 to allow the anvil 110210 to move from the open position toward the closed position. Similarly, defeating the first lockout 110208 rotates the tab 110236 out of the aperture 110221 such that the firing driver 110214 is not prevented from moving through the firing stroke via the first lockout 110208.

The compatible and ready-to fire staple cartridge 110250 further includes a second key (not shown, but similar to other keys shown and described elsewhere herein) that extends proximally therefrom. As the staple cartridge 110250 is removably positioned in the elongate channel 110209, the second key slides under the lockout body 110224 to hold the lockout body 110224 in the upward, unlocked position. With the lockout body 110224 in the upward, unlocked position, as described above, the lockout body 110224 is rotated upward and away from the hooks 110244 such that the lockout body 110224 and the firing driver 110214 are able to traverse past the hooks 110244 without being “caught”. In some embodiments, the staple cartridge 110250 comprises a sled that comprises the second key. In one aspect, the spring 110240 and the lockout body 110224 can be considered a ready-to-fire lockout in that a sled with a second key in its proximal, unfired positioned is required to prevent the pins 110225 on the lockout body 110224 from falling and being “caught” by the spring 110240. An absent second key from a staple cartridge can be indicative of, for example, a staple cartridge that does not have a compatible sled (a sled that in the proximal position, but does not include a second key) or a sled with a second key not being in an unfired position (a sled being at a partially spent or spent position).

It should be understood that, in a scenario where a staple cartridge that comprises the first key 110252, but not the second key, is removably positioned in the elongate channel 110209, the first lockout 110208 would be unlocked to allow for the anvil 110210 to move to the closed position. However, despite the tab 110236 being rotated out of the aperture 110221, should a user attempt to drive the firing driver 110214 through the firing stroke, the lockout body 110224 would have no second key to maintain it in the upward, unlocked position. Therefore, the lockout body 110224 would fall to the downward locked position and the pins 110225 would be “caught” by the hooks 110244 of the spring 110240, preventing distal motion of the firing driver 110214. Accordingly, in some embodiments, the surgical stapling system 110200 comprises two lockouts—a first lockout (a compatible staple cartridge lockout 110208) that serves as a closure and firing lockout and a second lockout (a ready-to-fire staple cartridge lockout comprised of lockout body 110224 and spring 110240) that serves as a secondary firing lockout.

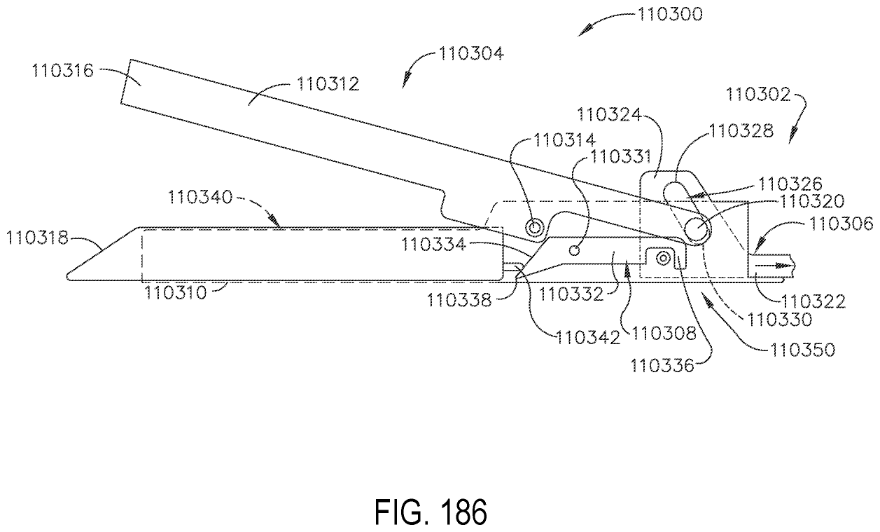

Referring now to FIG. 186, a surgical stapling system 110300 is provided, according to at least one aspect of the present disclosure. The surgical stapling system 110300 comprises a shaft 110302, an end effector 110304 extending from the shaft 110302, and a closure actuator 110306 extending through the shaft 110302. In some embodiments, the shaft 110302 is similar to shaft 110002. In some embodiments, the end effector 110304 is similar to end effector 110004.

The end effector 110304 comprises an elongate channel 110310, an anvil 110312, and a pivot 110314 pivotably coupling the anvil 110312 to the elongate channel 110310. In at least one arrangement, the pivot 110314 comprises a pin. The anvil 110312 is rotatable relative to the elongate channel 110310 between an open, locked position, where a distal tip 110316 of the anvil 110312 is rotated a first distance away from a distal tip 110318 of the elongate channel 110310, an open, unlocked position (similar to FIG. 179), where the distal tip 110316 of the anvil 110312 is rotated a second distance smaller than the first distance away from the distal tip 110318 of the elongate channel 110310, and a closed position (similar to FIG. 178) where the distal tip 110316 of the anvil 110312 is a third distance smaller than the second distance away from the distal tip 110318 of the elongate channel 110310. The anvil 110312 further comprises a pin 110320 extending from a proximal end thereof.

The closure actuator 110306 comprises an elongate bar 110322 extending along a first axis and a flange 110324 extending distally therefrom. The flange 110324 defines a slot 110326 that receives the pin 110320 and extends along a second axis that is transverse to the first axis of the elongate bar 110322. The slot 110326 comprises a first, distal end 110328 and a second, proximal end 110330 opposite the first, distal end 110328. The pin 110320 is movable along the slot 110326 between a first position, corresponding to the open, locked position of the anvil 110312, a second position, corresponding to the open, unlocked position of the anvil 110312, and a third position, corresponding to the closed position of the anvil 110312. The second position of the pin 110320 is closer to the first end 110328 of the slot 110326 than the first position of the pin 110320. The third position of the pin 110320 is closer to the first end 110328 of the slot 110326 than the second position of the pin 110320. In some embodiments, the first position of the pin 110320 is a position where the pin 110320 engages the second end 110330 of the slot 110326. In some embodiments, the third position of the pin 110320 is a position where the pin 110320 engages the first end 110328 of the slot 110326. In some embodiments, the first position and the second position of the pin 110320 are positions intermediate the first end 110328 of the slot 110326 and the second end 110330 of the slot 110326.

The closure actuator 110306 is movable through from a first, distal position toward a second, proximal position during a closure stroke by a drive system to move the pin 110320 along the slot 110326, and therefore, rotate the anvil 110312 from the open, unlocked position toward the closed positon. Notably, however, in the open, locked position of the anvil 110312, the closure actuator 110306 is unable to rotate the anvil 110312 toward the closed position, as explained in more detail below. In various embodiments, the drive system that drives the closure actuator 110306 comprises a motor-driven system. In various embodiments, the drive system that drives the closure actuator 110306 comprises a closure trigger and the closure actuator 110306 is manually movable according to motions provided to the closure trigger by a user.

In the open, locked position of the anvil 110312, the pin 110320 is in the first position within the slot 110326. In the first position, the flange 110324 is unable to apply a sufficient torque to the pin 110320 to cause the pin 110320 to move along the slot 110326 toward the first end 110328 thereof, and therefore, rotate the anvil 110312 away from the open, locked position. Stated another way, the closure actuator 110306 is prevented from moving proximally through its closure stroke with the pin 110320 in the first position. Accordingly, in one aspect, the slot 110326 of the flange 110324 and the pin 110320 collectively define a closure lockout 110350.

In various embodiments, as shown in FIG. 186, the lockout 110350 is positioned proximal to the pivot 110314. By moving the lockout 110350 proximal to the pivot 110314, space is freed up within the end effector 110304 and the lockout 110350 is moved away from the pinch point between the elongate channel 110310 and the anvil 110312.

The closure lockout 110350 is configurable between a locked state a locked state and an unlocked state. In one aspect, the locked state corresponds to the open, locked position of the anvil 110312, described above, in which the pin 110320 is positioned within the slot 110326 such that the closure actuator 110306 is prevented from moving the anvil 110312 toward the closed position. In one aspect, the unlocked state corresponds to the open, unlocked position of the anvil 110312, described above, in which the pin is positioned within the slot 110326 such that the closure actuator 110306 is permitted to move the anvil 110312 toward the closed position.

To transition the anvil 110312 from the open, locked position (the first position of the pin 110320) to the open, unlocked position (the second position of the pin 110320), an unlocking bar 110308 is utilized. The unlocking bar 110308 comprises a body 110332 pivotably coupled to the elongate channel 110310 with a pin 110331, a cam surface 110334 extending distally from the body 110332, and an arm 110336 extending proximally from the body 110332. The arm 110336 extends a length from the body 110332 such that, as the unlocking bar 110308 is rotated (counterclockwise as shown in FIG. 186), the arm 110336 engages the proximal end of the anvil 110312 to drive or “kick” the pin 110320 from its first position to its second position, thereby transitioning the anvil 110312 from the open, locked position to the open, unlocked position and the closure lockout 100350 from the locked state to the unlocked state.

To rotate the unlocking bar 110308, as described above, a compatible staple cartridge 110340 is positioned within the elongate channel 110310. The compatible staple cartridge 110340 comprises a key 110342 that engages the cam surface 110334 of the unlocking bar 110308 at a location that is distal to the pivot 110314 as the compatible staple cartridge 110340 is positioned in the elongate channel 110310. In some embodiments, the staple cartridge 110340 comprises a cartridge body that comprises the key 110342. In some embodiments, the staple cartridge 110340 comprises a retainer that comprises the key 110342. In some embodiments, the staple cartridge 110340 comprises a sled that comprises the key 110342.

In the illustrated arrangement, the point of contact between key 110342 and the cam surface 110334 is distal to the pivot 110314. In other embodiments, like the closure lockout 100350, the unlocking bar 110308 is also positioned proximal to the pivot 110314, freeing up additional space within the end effector. In embodiments where the unlocking bar 110308 is also positioned proximal to the pivot 110314, the key 110342 comprises a length to extend proximally from the staple cartridge 110340, past the pivot 110314, to engage the cam surface 110334 at a location proximal to the pivot 110314.

As the staple cartridge 110340 is moved proximally to seat the staple cartridge 110340 in the elongate channel 110310, the key 110342 slides along the cam surface 110334, rotating the unlocking bar 110308. As the unlocking bar 110308 rotates, the arm 110336 of the unlocking bar 110308 engages the proximal end of the anvil 110312 to move the pin 110320 within the slot 110326 toward the second position, transitioning the anvil 110312 to the open, unlocked position and the closure lockout 110350 to the unlocked state. The unlocking bar 110308 further comprises a tip 110338 that engages the base of the elongate channel 110310 to prevent the key 110342 of the staple cartridge 110340 from over rotating the unlocking bar 110308 and moving the pin 110320 beyond its second position. In various embodiments, the surgical stapling system 110300 further comprises a spring that biases the tip 110338 away from the base of the elongate channel 110310 and the arm 110336 away from the pin 110320.

With the pin 110320 in the second position within the slot 110326, the closure actuator 110306 is able to apply a sufficient torque to the pin 110320 to cause the pin 110320 to move within the slot 110326 toward the first end 110328 thereof, and therefore, rotate the anvil 110312 from the open, unlocked position toward the closed position. Accordingly, in the unlocked state of the closure lockout 110350, the closure actuator 110306 is permitted to move proximally through its closure stroke to place the anvil 110312 in the closed position. Once the anvil 110312 is in the closed position, a clinician is able to perform a staple firing function to deploy the staples from the staple cartridge, as described elsewhere herein.

In various embodiments, the surgical stapling system 110300 further comprises a firing actuator, such as firing actuator 110106 (described above) or firing actuator 110206 (described above), that is configured to move through a firing stroke after the closure actuator 110306 has placed the anvil 110312 in the closed position to deploy the staples from the staple cartridge 110340. In some such embodiments, the surgical stapling system 110300 further comprises a firing lockout, such as lockout 110108 or lockout 110260, that prevents the firing driver of the firing actuator from advancing through the staple cartridge 110340 unless the staple cartridge 110340 is a compatible and/or ready-to-fire staple cartridge. Accordingly, in some such embodiments, the surgical stapling system 110300 comprises two lockouts-one that prevents the anvil 110312 from rotating toward the closed position (the closure lockout 110350) and another that prevents the firing actuator from advancing through its firing stroke unless a compatible and/or ready-to-fire staple cartridge is positioned within the end effector (such as lockout 110108 or lockout 110260). In various embodiments, the staple cartridge 110340 comprises two keys that independently transition each lockout—the closure lockout 110350 and the firing lockout—to their respective unlocked states. In various other embodiments, the key 110342 transitions both the closure lockout 110350 and the firing lockout to their respective unlocked states.

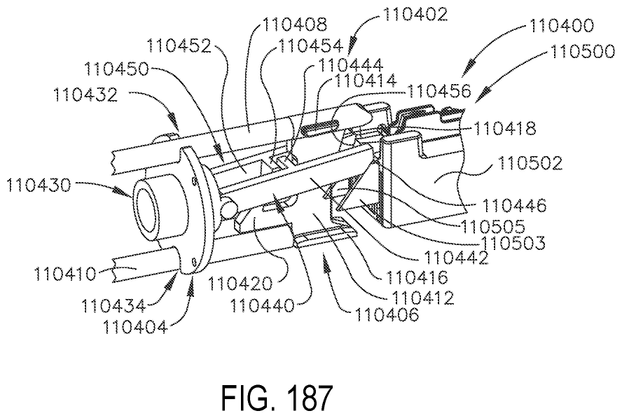

Referring now to FIGS. 187-189, a surgical stapling system 110400 is provided, according to at least one aspect of the present disclosure. The surgical stapling system 110400 comprises a firing actuator 110402 and a lockout 110404.

The firing actuator 110402 comprises a firing driver 110406, a first push rod 110408 attached at an upper end of the firing driver 110406, and a second push rod 110410 attached at a lower end of the firing driver 110406. The first push rod 110408 and the second push rod 110410 can be driven using any suitable drive system, such as a motor drive system, to drive the firing driver 110406 from a proximal position toward a distal position. An exemplary motor drive system is described in U.S. Patent Application Publication No. 2015/0173756, entitled SURGICAL CUTTING AND STAPLING METHODS, filed Dec. 23, 2013, which is hereby incorporated by reference in its entirety herein. The firing driver 110406 comprises a body 110412, a first cam 110414, a second cam 110416, a knife 110418, and a hook 110420, as will be described in more detail below.

In one aspect, the surgical stapling system 110400 further comprises an end effector (similar to end effector 110004) that comprises an elongate channel (similar to elongate channel 110007), an anvil (similar to anvil 110008), and a pivot that pivotably couples the anvil to the elongate channel about a closure axis (similar to closure axis CA). In one aspect, the pivot comprises a pin. The anvil is pivotable relative to the elongate channel about the closure axis between an open position (similar to FIG. 179) and a closed position (similar to FIG. 178). It should be noted that the end effector of the surgical stapling system 110400 is hidden in FIGS. 187-189 to help properly illustrate the firing actuator 100402 and the lockout 110404, described in more detail below.

In operation, the firing driver 110406 is movable through a stroke that includes a closure stroke portion and a firing stroke portion distal to the closure stroke portion. During the closure stroke portion, the first cam 110414 of the firing driver 110406 engages a ramp on the anvil to cam the anvil from the open position toward the closed position. During the firing stroke portion subsequent and distal to the closure stroke portion, the body 110412 of the firing driver 110406 engages and drives a sled, similar to sled 110026, through a staple cartridge, such as staple cartridge 110010, to deploy the staples therefrom while the knife 110418 cuts the stapled tissue. Furthermore, as the firing driver 110406 is advanced distally through the firing stroke portion, the second cam 110416 engages the elongate channel and the first cam 110414 engages the anvil to control the distance, or tissue gap, therebetween. In one aspect, since the firing actuator 110402 is utilized to move the anvil toward the closed position prior to deploying staples from a staple cartridge, the firing actuator 110402 can also be considered a closure actuator.

The lockout 110404 includes a flange 110430, a first lockout 110440 pivotably coupled to a first lateral side of the flange 110430, and a second lockout 110450 pivotably coupled to a second lateral side of the flange 110430. The flange 110430 defines a first recess 110432 that receives the first push rod 110408 and guides the first push rod 110408 during the closure stroke portion and the firing stroke portion. The flange 110430 further defines a second recess 110434 that receives the second push rod 110410 and guides the second push rod 110410 during the closure stroke portion and the firing stroke portion. In some embodiments, surgical stapling system 110400 further comprises a shaft, similar to shaft 110002, and the flange 110430 is mounted to the shaft.

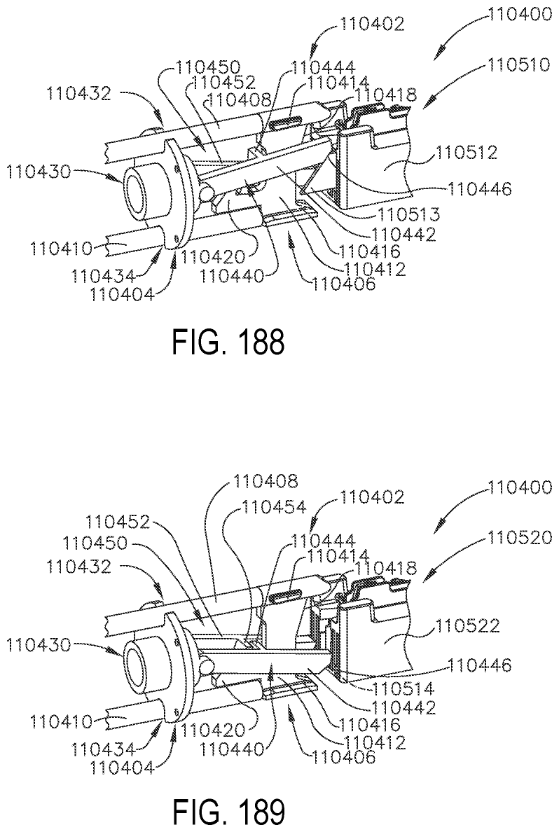

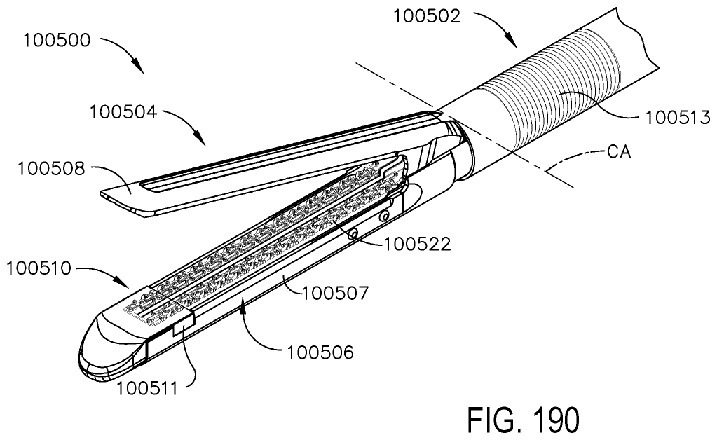

The first lockout 110440 comprises a first lockout arm 110442 pivotably coupled to the flange 110430 and a first tab 110444 that extends transversely from the first lockout arm 110442. The first lockout arm 110442 is pivotable relative to the flange 110430 between a first locked state, in which the first tab 110444 is received within the hook 110420 (FIG. 189) and a first unlocked state in which the first tab 110444 is rotated out of the hook 110420 (FIGS. 187 and 188). In some embodiments, a spring biases the first lockout 110440 toward the first locked state. In the first locked state, the first tab 110444 and the hook 110420 are longitudinally aligned such that, should a user attempt to drive the firing driver 110406 through the closure stroke portion and the firing stroke portion, the first tab 110444 would engage the hook 110420 at a location proximal with respect to the closure axis defined by the pivot, preventing distal motion of the firing driver 110406. In one aspect, the first lockout 110440 can be considered a compatible staple cartridge lockout as a compatible staple cartridge (i.e., a cartridge including a first authentication key) is required in order to transition the first lockout 110440 to the first unlocked position, as will be explained in more detail below.

Similarly, the second lockout 110450 comprises a second lockout arm 110452 pivotably coupled to the flange 110430 and a second tab 110454 that extends transversely from the second lockout arm 110452 toward the first lockout arm 110442. The second lockout 110450 is pivotable relative to the flange 110430 between a second locked state, in which the second tab 110454 is received within the hook 110420 (FIGS. 188 and 189) and a second unlocked state in which the second tab 110454 is rotated out of the hook 110420 (FIG. 187). In some embodiments, a spring biases the second lockout 110450 toward the second locked state. In some embodiments, the same spring that biases the first lockout 110440 toward the first locked state also biases the second lockout 110450 toward the second locked state. In the second locked state, the second tab 110454 and the hook 110420 are longitudinally aligned such that, should a user attempt to drive the firing driver 110406 through the closure stroke portion and the firing stroke portion, the second tab 110454 would engage the hook 110420 at a location proximal with respect to the closure axis defined by the pivot, preventing distal motion of the firing driver 110406. In one aspect, the second lockout 110450 can be considered a read-to-fire staple cartridge lockout as a staple cartridge that is unspent is required to transition the second lockout to the second unlocked position, as will be explained in more detail below.

In various embodiments, a majority, if not all, of the lockout 110404 is positioned proximal with respect to the pivot between the anvil and the elongate channel. In particular, as described above, the point of contact between the firing actuator 110402 (the hook 110420) and the lockout 110404 (the first tab 110444 and/or the second tab 110454) is proximal with respect to the pivot. By moving the lockout 110404 proximal of the pivot, space is freed up within the end effector and the lockout 110404 is moved away from the pinch point between the elongate channel and the anvil. In various embodiments, the entire lockout 110404 is positioned proximal of the pivot, freeing up additional space within the end effector.

In use, the lockout 110404 prevents advancement of the firing driver 110406 unless a compatible and ready-to-fire staple cartridge is positioned in an elongate channel, such as elongate channel 110007, of the surgical stapling system 110400. In various embodiments, the staple cartridge can include two discrete authentication keys where a first authentication key is configured to transition the first lockout 110440 from the first locked state to the first unlocked state and a second authentication key is configured to transition the second lockout 110450 from the second locked state to the second unlocked state. With the first lockout 110440 and the second lockout 110450 in their respective unlocked positions, the firing driver 110406 is permitted to advance through the closure stroke and the firing stroke. However, if one or both of the first lockout 110440 and the second lockout 110450 is in its respective locked position, the firing driver 110406 is prevented from advancing through the closure stroke portion and the firing stroke portion. In one aspect, since the lockout 110404 prevents the firing driver 110406 from moving through the closure stroke portion and the firing stroke portion when one or both of the first lockout 110440 and the second lockout 110450 is in its respective locked position, the lockout 110404 can be considered a closure lockout and/or a firing lockout.

Referring now to FIG. 187, a staple cartridge 110500 is provided that includes a cartridge body 110502 and a sled (not shown, but similar to sled 110026). The cartridge body 110502 includes a first key 110503 and the sled comprises a second key 110505. As the staple cartridge 110500 is positioned in the elongate channel, the first key 110503 engages a cam surface 110446 on the first lockout arm 110442 to rotate the first lockout 110440 from the first locked state toward the first unlocked state. As the staple cartridge 110500 is moved proximally to seat the staple cartridge 110500 in the elongate channel, the first key 110503 clears the cam surface 110446 and then engages first lockout arm 110442 to continue camming the first lockout 110440 toward the first unlocked state, and thus, moving the first tab 110444 out of the hook 110420. In some embodiments, the point of contact between the first key 110503 and the cam surface 110446 is distal with respect to the pivot between the anvil and the elongate channel of the end effector. In some embodiments, the point of contact between the first key 110503 and the cam surface 110446 is proximal with respect to the pivot between the anvil and the elongate channel of the end effector. In some such embodiments, the first key 110503 comprises a length that allows the key 110503 to extend proximally from the staple cartridge 110500, past the pivot, to engage the cam surface 110446 at a location proximal to the pivot.

Similarly, as the staple cartridge 110500 is positioned in the elongate channel, the second key 110505 engages a cam surface 110456 on the second lockout arm 110452 to rotate the second lockout 110450 from the second locked state toward the second unlocked state. As the staple cartridge 110500 is moved proximally to seat the staple cartridge 110500 in the elongate channel, the second key 110505 clears the cam surface 110456 and then engages the second lockout arm 110452 to continue camming the second lockout 110450 toward the second unlocked state, and thus, moving the second tab 110454 out of the hook 110420. With both lockouts 110440, 110450 rotated to their respective unlocked positions, the firing driver 110406 is permitted to move through the closure stroke portion and the firing stroke portion. In some embodiments, the point of contact between the second key 110505 and the cam surface 110456 is distal with respect to the pivot between the anvil and the elongate channel of the end effector. In some embodiments, the point of contact between the second key 110505 and the cam surface 110456 is proximal with respect to the pivot between the anvil and the elongate channel of the end effector. In some such embodiments, the second key 110505 comprises a length that allows the key 110505 to extend proximally from the staple cartridge 110500, past the pivot, to engage the cam surface 110456 at a location proximal to the pivot.

Referring now to FIG. 188, the staple cartridge 110500 has been replaced with a first replacement staple cartridge 110510 that includes a cartridge body 110512 that includes a first key 100513 and a sled (not shown, but similar to sled 110026) that notably does not comprise a second key. An absent second key from the first replacement staple cartridge 110510 can be indicative of, for example, a staple cartridge that does not have a compatible sled (a sled that in the proximal position, but does not include a second key) or a sled with a second key not being in an unfired position (a sled being at a partially spent or spent position). Accordingly, when the first replacement staple cartridge 110510 is removably positioned in the elongate channel, the first lockout 110440 is transitioned to the first unlocked state (via the first key 110513, as described above) but the second lockout 110550 remains in the locked state. In such a state, the lockout 110404 prevents distal motion of the firing driver 110406. As outlined above, only one of the lockouts 110440, 110450 needs to be in its respective locked state to prevent the firing driver 110406 from moving through the closure stroke portion and the firing stroke portion.

Referring now to FIG. 189, the first replacement staple cartridge 110510 has been replaced with a second replacement staple cartridge 110520 that includes a cartridge body 110522 that notably does not comprise a first key and a sled (not shown, but similar to sled 110026) that notably does not comprise a second key. As referenced above, an absent second key from the second replacement staple cartridge 110520 can be indicative of, for example, a staple cartridge that does not have a compatible sled (a sled that in the proximal position, but does not include a second key) or a sled with a second key not being in an unfired position (a sled being at a partially spent or spent position). In addition, an absent first key from the second replacement staple cartridge 110520 can be indicative of the second replacement staple cartridge 110520 being an incompatible staple cartridge. Accordingly, when the second replacement staple cartridge 110520 is removably positioned in the elongate channel, the first lockout 110440 and the second lockout 110450 remain in their respective locked states, preventing distal motion of the firing driver 110406 through the closure stroke portion and the firing stroke portion.

In all of the various embodiments disclosed herein wherein a key is formed on or otherwise provided on the sled of an otherwise compatible cartridge, persons of ordinary skill in the art will understand that the sled must be in an unfired, proximal-most position in the cartridge in order for the key to unlockingly interact with the lockout of the surgical instrument. If for example, a sled that otherwise includes an appropriate unlocking key has been moved distally out of the unfired, proximal-most position indicating that at least some of the staples have been fired and are missing (by the distal advancement of the sled), would be out of position to unlockingly engage the corresponding lockout. Such systems would thereby prevent the use of a previously fired or partially fired staple cartridge.

Referring now to the Figures, a surgical stapler 100500 is shown that includes a shaft 100502 and an end effector 100504 extending from the shaft 100502. The end effector 100504 includes a first jaw 100506 and a second jaw 100508. The first jaw 100506 includes an elongate channel 100507 and a staple cartridge 100510. The staple cartridge 100510 is insertable into and removable from the elongate channel 100507. In particular, the staple cartridge 100510 includes tabs 100511 on each lateral side thereof that snap into corresponding recesses 100509 defined in the elongate channel 100507. Other embodiments are envisioned in which the staple cartridge 100510 is integral with, or not removable from, the first jaw 100506.

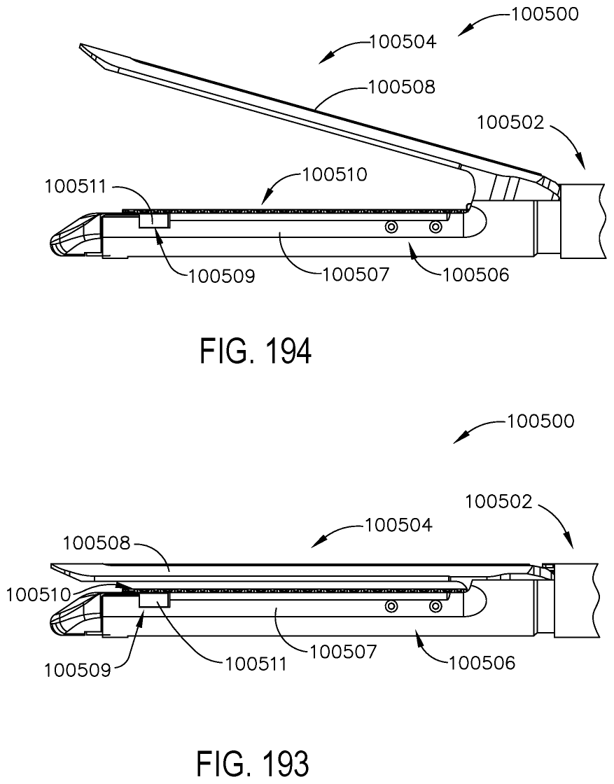

The second jaw 100508 includes an anvil configured to deform staples ejected from the staple cartridge 100510. The second jaw 100508 is pivotable relative to the first jaw 10056 about a closure axis CA between an open position (FIG. 194) and a closed position (FIG. 193); however, other embodiments are envisioned in which the first jaw 100506 is pivotable relative to the second jaw 100508. The surgical stapler 100500 further includes an articulation joint 100513 configured to permit the end effector 100504 to be rotated, or articulated, relative to the shaft 100502. Other embodiments are envisioned which do not include an articulation joint.

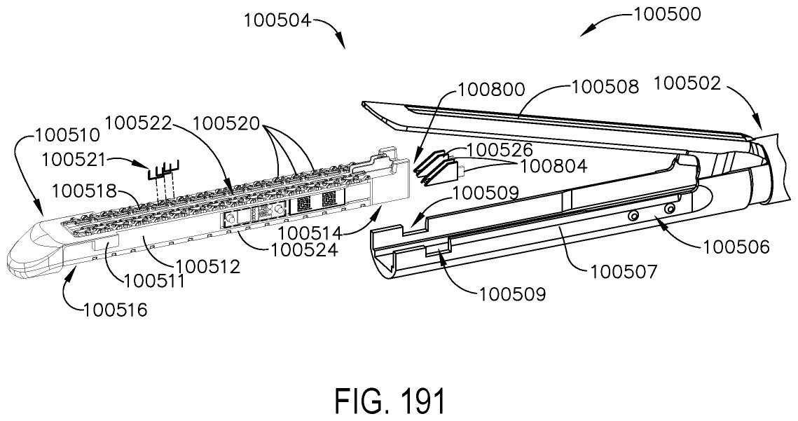

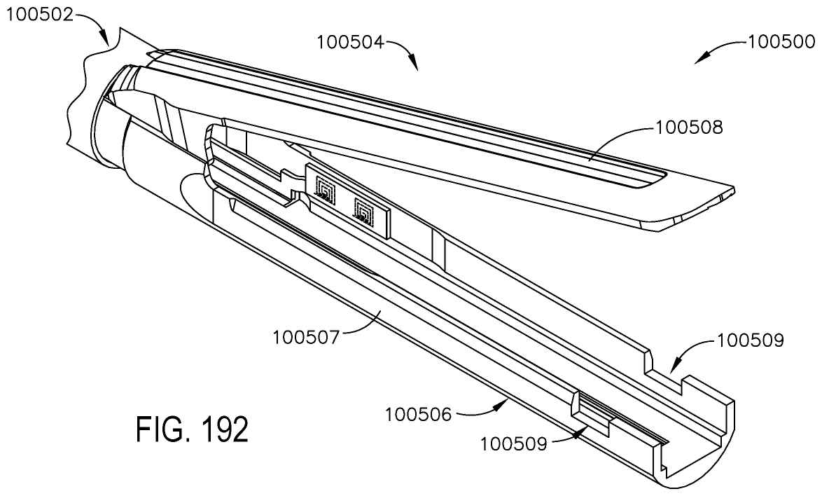

As shown in FIG. 191, the staple cartridge 100510 includes a cartridge body 100512. The cartridge body 100512 includes a proximal end 100514, a distal end 100516, and a deck 100518 extending between the proximal end 100514 and the distal end 100516. In use, the staple cartridge 100510 is positioned on a first side of the tissue to be stapled and the anvil 100508 is positioned on a second side of the tissue. The anvil 100508 is moved toward the staple cartridge 100510 (see FIGS. 193 and 194) to compress and clamp the tissue against the deck 100518. Thereafter, staples 100521 removably stored in the cartridge body 100512 can be deployed into the tissue. The cartridge body 100512 includes staple cavities 100520 defined therein wherein the staples 100521 are removably stored in the staple cavities 100520. The staple cavities 100520 are arranged in six longitudinal rows. Three rows of staple cavities are positioned on a first side of a longitudinal slot 100522 and three rows of staple cavities are positioned on a second side of the longitudinal slot 100522. Other arrangements of staple cavities and staples may be possible.

The staples are supported by staple drivers in the cartridge body 100512. Staples supported on staple drivers can be seen in U.S. Patent Application Publication No. 2021/0059672, entitled “SURGICAL INSTRUMENT WITH WIRELESS COMMUNICATION BETWEEN A CONTROL UNIT OF A ROBOTIC SYSTEM AND REMOTE SENSOR”, filed Sep. 14, 2020, which is hereby incorporated by reference in its entirety herein. The drivers are movable between a first, or unfired position, and a second, or fired, position to eject the staples from the staple cavities 100520. The drivers are retained in the cartridge body 100512 by a retainer 100524 which extends around the bottom of the cartridge body 100512 and includes resilient members configured to grip the cartridge body 100512 and hold the retainer 100524 to the cartridge body 100512. The drivers are movable between their unfired positions and their fired positions by a sled 100526. The sled 100526 is movable between a proximal position adjacent the proximal end 100514 and a distal position adjacent the distal end 100516. The sled 100526 includes a plurality of ramped surfaces configured to slide under the drivers and lift the drivers, and the staples 100521 supported thereon, toward the anvil 100508. In various embodiments, the staples 100521 are not supported by staple drivers, but rather, the staples include integral drive surfaces that are directly engaged by the sled 100526 to lift the staples, examples of which are described in U.S. Patent Application Publication No. 2015/0173756, entitled “SURGICAL CUTTING AND STAPLING METHODS”, filed Dec. 23, 2013, which is hereby incorporated by reference in its entirety herein.

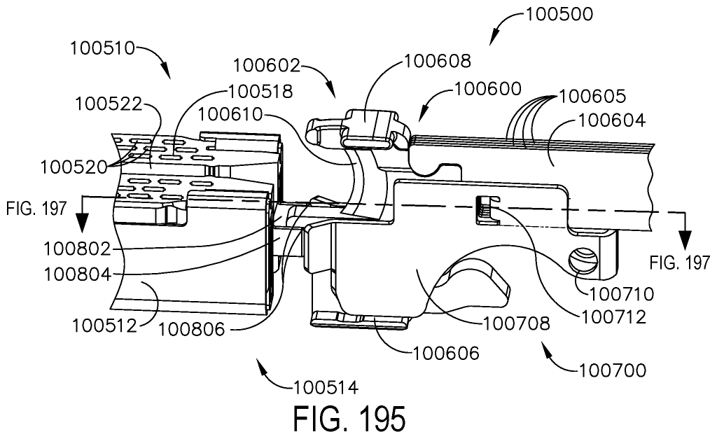

Further to the above, the sled 100526 is moved distally by a firing assembly 100600 (see FIG. 195) that includes a firing member or firing beam 100602 and a firing bar 100604 extending proximally from the firing beam 100602. The firing beam 100602 is configured to contact the sled 100526 and push the sled 100526 toward the distal end 100516 during a firing stroke. The firing bar 100604 includes a plurality of laminate strips 100605. The longitudinal slot 100522 defined in the cartridge body 100512 is configured to accommodate the firing beam 100602 and the firing bar 100604 during the firing stroke of the firing assembly 100600. The anvil 100508 also includes a slot defined therein that receives the firing beam 100602. The firing beam 100602 further includes a first cam 100606 which engages the first jaw 100506 and a second cam 100608 which engages the second jaw 100508 in the slot of the anvil. As the firing beam 100602 is advanced distally through the firing stroke, the first cam 100606 and the second cam 100608 control the distance, or tissue gap, between the deck 100518 of the staple cartridge 100510 and the anvil 100508. The firing beam 100602 also includes a knife 100610 configured to incise the tissue captured intermediate the staple cartridge 100510 and the anvil 100508. It is desirable for the knife 100610 to be positioned at least partially proximal to the ramped surfaces such that the staples are ejected ahead of the knife 100610.

In various embodiments, closure of the end effector 100504 is performed separately and distinctly from the firing of the staples 100521. In some such embodiments, the anvil 100508 includes a ramp on a proximal end thereof, the ramp engageable by a closure tube that is movable distally to cam the anvil 100508 to the closed position. To transition the anvil 100508 to the open position, the closure tube is retracted proximally, and springs positioned within the end effector 100504 bias the anvil 100508 to the open position. In various other embodiments, the anvil 100508 includes a tab and the closure tube defines an aperture which engages the tab as the closure tube moves proximally, thereby positively transitioning the anvil 100508 to the open position. Exemplary closure mechanisms are described in U.S. Patent Application Publication No. 2021/0059672, entitled “SURGICAL INSTRUMENT WITH WIRELESS COMMUNICATION BETWEEN A CONTROL UNIT OF A ROBOTIC SYSTEM AND REMOTE SENSOR”, filed Sep. 14, 2020, which is hereby incorporated by reference in its entirety herein.

In various other embodiments, the anvil 100508 is moved from the open position to the closed position using the firing beam 100602. In some such embodiments, the anvil 100508 includes a ramp that extends proximally from the slot defined therein that is engaged by the second cam 100608 of the firing beam 100602 during a first, closure stroke portion of the firing beam 100602 to move the anvil 100508 to the closed position. In some embodiments, the first closure stroke portion of the firing beam can be about 0.105″-0.250″ of the firing beam 100602 travel. At the end of the first closure stroke portion of the firing beam 100602, the firing beam 100602 can continue advancing distally through a second firing stroke portion to deploy staples from the staple cartridge 100510 and incise tissue captured by the end effector with the knife 100610, as described above. Exemplary firing beams that close the anvil and fire staples are described in U.S. Pat. No. 11,160,551, entitled “ARTICULATABLE SURGICAL STAPLING INSTRUMENTS”, filed Dec. 21, 2016, which is hereby incorporated by reference in its entirety herein. In one aspect, when a firing beam is utilized to move the anvil 100508 toward the closed position and deploy staples from the staple cartridge 100510, the firing beam can be considered both a closure system and/or a firing assembly.

After a staple cartridge 100510 has been fired, or at least partially fired, it is removed from the elongate channel 100507 and then replaced with another replaceable staple cartridge, if desired. At such point, the surgical stapler 100500 can be re-used to continue stapling and incising the patient tissue. In some instances, however, a previously-fired staple cartridge can be accidentally loaded into the elongate channel 100507. If the firing beam 100602 were to be advanced distally within such a previously-fired staple cartridge, the surgical stapler 100500 would cut the patient tissue without stapling it. The surgical stapler 100500 would similarly cut the patient tissue without stapling it if the firing beam 100602 were advanced distally through a staple firing stroke without a staple cartridge positioned in the elongate channel 100507 at all. In addition, various surgical staple cartridges may have different arrays of and/or orientations of staples/fasteners therein. The sizes of the staples or fasteners, as well as the number of fasteners may vary from cartridge type to cartridge type depending upon a particular surgical procedure or application. To ensure that the staples are properly crimped or formed, the surgical staple cartridges must be used in connection with corresponding, compatible anvils that have the proper array of staple-forming pockets therein as well as the proper cutting and firing components. Should a “non-compatible” cartridge be loaded into a surgical stapling device that has an anvil that is mismatched to the staple cartridge, the staples may not be properly formed during the firing process.

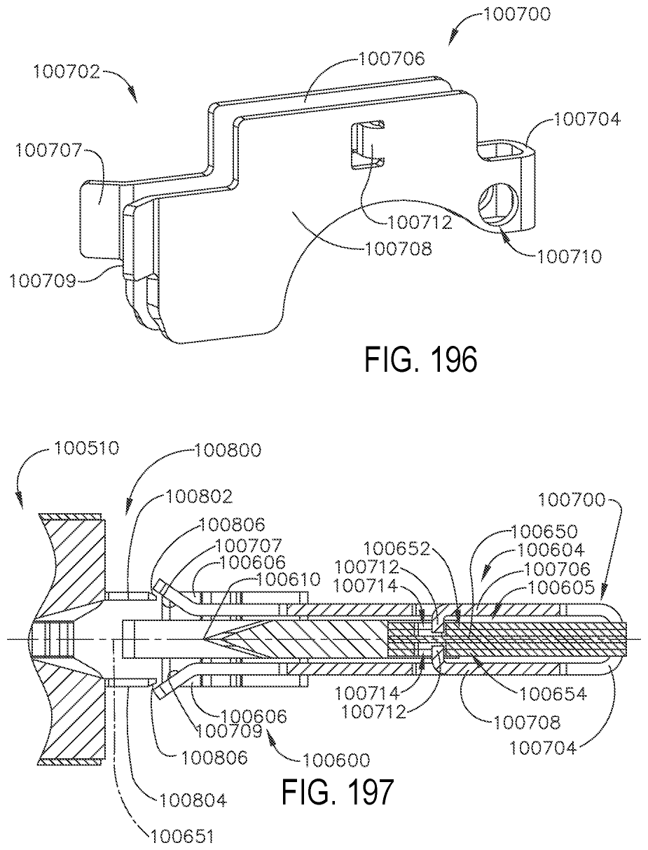

To this end, the surgical stapler 100500 further includes a lockout 100700 that is configured to prevent the firing assembly 100600 from moving distally through the firing stroke unless an authorized or compatible staple cartridge is operably seated in the elongate channel 100507. Referring to FIGS. 195 and 196, the lockout 100700 includes a single, bi-lateral lockout spring 100702. In various embodiments, the spring 100702 is supported, or at least partially supported, in the elongate channel 100507. In various embodiments, the spring 100702 is supported, or at least partially supported, in the shaft 100502. In various embodiments, the spring 100702 is attached to a shaft mount flange, similar to the shaft mount flange discussed in U.S. Pat. No. 11,298,129, entitled “METHOD FOR PROVIDING AN AUTHENTICATION LOCKOUT IN A SURGICAL STAPLER WITH A REPLACEABLE CARTRIDGE”, filed Jun. 26, 2019, which is hereby incorporated by reference in its entirety herein. In various other embodiments, the spring 100702 is directly attached to the elongate channel 100507.

The lockout spring 100702 includes a central body portion 100704, a first lockout arm 100706 extending from a first side of the central body portion 100704, and a second lockout arm 100708 extending from a second side of the central body portion 100704. The spring 100702 is supported in the elongate channel 100507 and affixed to the shaft mount flange or elongate channel 100507 by a pin that extends through holes in the shaft mount flange or elongate channel 100507 and through holes 100710 in the first lockout arm 100706 and the second lockout arm 100708. As seen in FIG. 195, the firing bar 100604 extends above the central body portion 100704.

The first lockout arm 100706 and the second lockout arm 100708 include projections 100712 that extend transversely therefrom. The projections 100712 are receivable in corresponding notches 100714 defined in the plurality of laminate strips 100605. In various embodiments, the plurality of laminated strips 100605 includes a central laminated strip 100650 that defines a firing bar axis 100651, a first subset of laminated strips 100652 that extend along a first lateral side of the central laminated strip 100650, and a second subset of laminated strips 100654 that extend along a second lateral side of the central laminated strip 100650. In some embodiments, the notch 100714 that receives the projection 100712 from the first lockout arm 100706 extends through some, or all, of the first subset of laminated strips 100652. Similarly, the notch 100714 that receives the projection 100712 from the second lockout arm 100708 extends through some, or all, of the second subset of laminated strips 100654. In some such embodiments, a notch is absent from the central laminated strip 100650, thus preventing the tips of the projections 100712 from engaging one another. In various other embodiments, a notch is also defined in the central laminated strip 100650 such that an aperture is defined through all of the plurality of laminated strips 100605. In some such embodiments, the projection 100712 from the first lockout arm 100706 and the projection 100712 from the second lockout arm 100708 extend within the aperture and engage one another at, or around, the firing bar axis 100651.

As referenced above, the firing lockout 100700 is configured to prevent the firing assembly 100600 from moving distally through the firing stroke unless an authorized or compatible staple cartridge is operably seated in the elongate channel 100507. The firing lockout 100700 is configurable between a locked state and an unlocked state. In the locked state, the projection 100712 from the first lockout arm 100706 is received in the notch 100714 in the first subset of laminated strips 100652 and the projection 100712 from the second lockout arm 100708 is received in the notch 100714 in the second subset of laminated strips 100654, as shown in FIGS. 195 and 197. In the locked state, the projections 100712 engage the firing bar 100604 to prevent distal movement of the firing assembly 100600. In the unlocked state, the projection 100712 from the first lockout arm 100706 is pivoted laterally away, or displaced, from the notch 100714 in the first subset of laminated strips 100652 and the projection 100712 from the second lockout arm 100708 is pivoted laterally away, or displaced, from, the notch 100714 in the second subset of laminated strips 100654. In the unlocked state, the projections 100712 permit distal movement distal movement of the firing assembly 100600.

As referenced above, the firing lockout 100700 includes a lockout spring 100702 that includes a central body portion 100704, a first lockout arm 100706 extending from a first side of the central body portion 100704, and a second lockout arm 100708 extending from a second side of the central body portion 100704. In a rested state of the lockout spring 100702, the projections 100712 of the first lockout arm 100706 and the second lockout arm 100708 are received in their corresponding notches 100714. Stated another way, the lockout spring 100702 naturally rests in the locked state. As the lockout 100700 is transitioned to the unlocked state, the projections 100712 are pivoted or otherwise moved away from their corresponding notches 100714, and therefore, the first lockout arm 100706 and second lockout arm 100708 are pivoted relative to the central body portion 100704. Given the inherent nature of springs, the first lockout arm 100706 and second lockout arm 100708 are naturally biased toward the resting state of the firing lockout 100700, and therefore, the firing lockout 100700 is naturally biased toward the locked state.

In one aspect, lockouts that engage the firing beam at locations that are laterally outward from the firing bar axis 100651 may be subject to “blowout”, due to an amount of torque that is applied by the firing assembly to the lockout. Shifting the engagement location between the lockout 100700 and the firing assembly 100600 to, or close to, the firing bar axis 100651 reduces, or eliminates, the amount of leverage that the firing assembly 100600 can apply to the lockout 100700, thus decreasing the chance of blowout of the lockout 100700 from occurring.

As discussed above, the lockout 100700 prevents the firing beam from moving distally from its proximal-most, starting position unless an authorized or compatible staple cartridge is operably seated in the elongate channel 100507. In various embodiments, an authorized or compatible staple cartridge includes a release key 100800 that is configured to transition the lockout 100700 from the locked state to the unlocked state.

In various embodiments, as shown FIG. 195, the authorized or compatible staple cartridge 100510 includes a release key 100800 that includes a first key 100802 and a second key 100804. In various embodiments, as shown more clearly in FIG. 191, the sled 100526 includes the first key 100802 and the second key 100804. In some such embodiments, the first key 100802 and the second key 100804 are sized to engage the lockout 100700 when the sled 100526 is in its proximal, unfired position, which is indicative of an unfired, or unspent, state cartridge (i.e., no staples have been deployed from the staple cartridge). Accordingly, positioning the release key 100800 on the sled 100526 both informs a clinician that, not only is the staple cartridge 100510 an authorized or compatible staple cartridge, but also that the staple cartridge 100510 is unspent and ready for firing. In some embodiments, the release key 100800 can be integrally molded with the cartridge body 100512. In other arrangements, the retainer 100524 includes the release key 100800. In embodiments where the cartridge body 100512 or the retainer 100524 include the release key 100800, the release key 100800 can be breakable from the staple cartridge by the clinician once the staple cartridge has been fired to ensure that lockout 100700 prevents a subsequent actuation of the firing assembly through the spent stable cartridge. The keys 100802, 100804 include tips 100806.

Referring again to FIG. 196, the first lockout arm 100706 includes a first cam surface 100707 on a distal end thereof and the second lockout arm 100708 includes a second cam surface 100709 on a distal end thereof. In operation, the authorized or compatible staple cartridge 100510 may be inserted, e.g. longitudinally, into the elongate channel 100507 so as to bring the tip 100806 of the first key 100802 into contact with the first cam surface 100707 on the first lockout arm 100706 and the tip 100806 of the second key 100804 into contact with the second cam surface 100709 on the second lockout arm 100708.

During the insertion of the authorized or compatible staple cartridge 100510 in a proximal direction into the elongate channel 100507, the tips 100806 on the first key 100802 and the second key 100804 pivot the first lockout arm 100706 and the second lockout arm 100708 of the lockout spring 100702 laterally outward, in opposite directions, causing the projections 100712 to pivot out of the corresponding notches 100714, transitioning the firing lockout 100700 to an unlocked state. After the firing lockout 100700 has been transitioned to the unlocked state, a user is free to actuate the firing assembly 100600 and drive the firing assembly through its firing stroke.

Accordingly, the firing lockout 100700 prevents the firing assembly 100600 from advancing through its firing stroke unless a compatible staple cartridge is positioned in the elongate channel 100507. As referenced above, the firing lockout 100700 provides the benefit of shifting the point of engagement between the firing lockout 100700 and the firing assembly 100600 at, or close to, the firing bar axis 100651 of the firing bar 100604, which reduces, or eliminates, the chance of the firing lockout 100700 blowing out when an incompatible staple cartridge is positioned in the elongate channel 100507.

While the firing lockout 100700 shown and described above includes two lockout arms 100706, 100708 for preventing movement of the firing assembly 100600 unless a compatible staple cartridge is positioned in the elongate channel 100507, other embodiments are envisioned where the firing lockout 100700 only includes one lockout arm for preventing movement of the firing assembly 100600. In some such embodiments, the firing lockout 100700 requires only one key on the staple cartridge to transition the lockout arm from the unlocked position to the locked position.

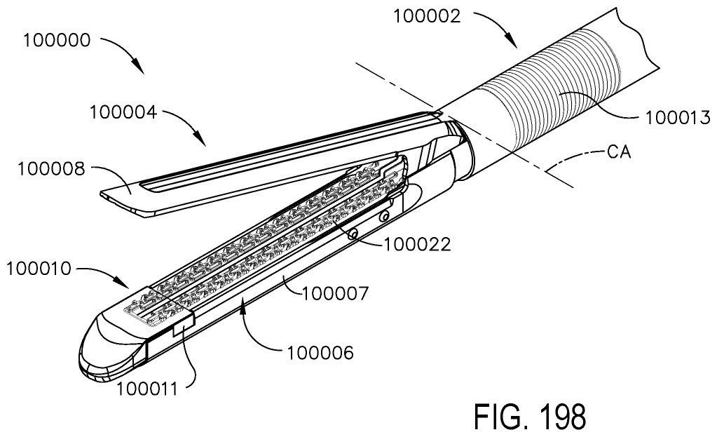

Referring to FIG. 198, a surgical stapling system 100000 comprises a shaft 100002 and an end effector 100004 extending from the shaft 100002. The end effector 100004 comprises a first jaw 100006 and a second jaw 100008. The first jaw 100006 comprises an elongate channel 100007 and a staple cartridge 100010. The staple cartridge 100010 is configured to be replaced during a surgical procedure. The staple cartridge 100010 comprises tabs 100011 on the lateral sides thereof that fit within corresponding recesses 100009 defined in the elongate channel 100007 and align the staple cartridge 100010 in the channel 100007. The staple cartridge 100010 comprises one or more lock shoulders that releasably engage the elongate channel 100007 and permit the staple cartridge 100010 to be removed from the channel 100007. Other embodiments are envisioned in which the staple cartridge 100010 is not removable from, or at least readily replaceable from, the first jaw 100006.

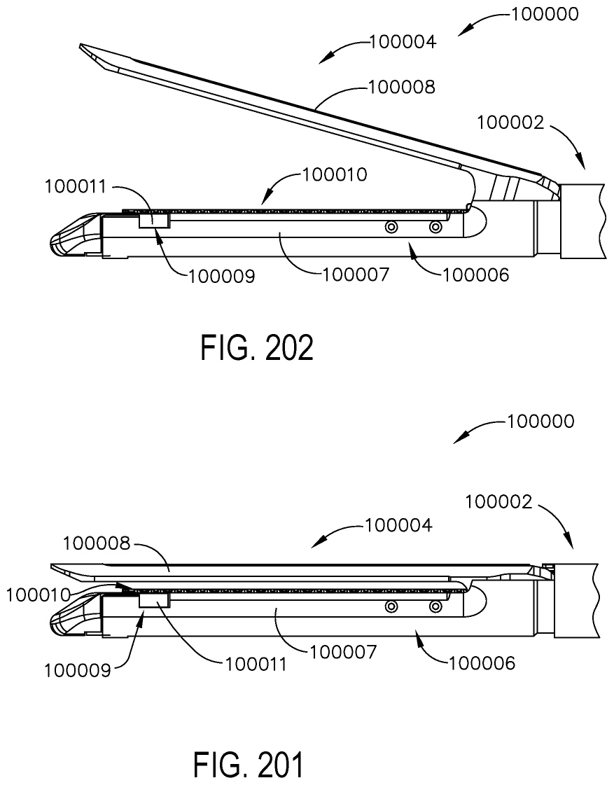

The second jaw 100008 comprises an anvil configured to deform staples ejected from the staple cartridge 100010. The second jaw 100008 is pivotable relative to the first jaw 10006 about a closure axis CA between an open position (FIG. 202) and a closed position (FIG. 201); however, other embodiments are envisioned in which the first jaw 100006 is pivotable relative to the second jaw 100008. The surgical stapling system 100000 further comprises an articulation joint 100013 configured to permit the end effector 100004 to be rotated, or articulated, relative to the shaft 100002. Other embodiments are envisioned which do not include an articulation joint.

The staple cartridge 100010 comprises a cartridge body 100012. The cartridge body 100012 includes a proximal end 100014, a distal end 100016, and a deck 100018 extending between the proximal end 100014 and the distal end 100016. In use, the staple cartridge 100010 is positioned on a first side of the tissue to be stapled and the anvil 100008 is positioned on a second side of the tissue. The anvil 100008 is moved toward the staple cartridge 100010 (see FIGS. 201 and 202) to compress and clamp the tissue against the deck 100018. Thereafter, staples 100021 removably stored in the cartridge body 100012 can be deployed into the tissue. The cartridge body 100012 includes staple cavities 100020 defined therein wherein staples are removably stored in the staple cavities 100020. The staple cavities 100020 are arranged in six longitudinal rows. Three rows of staple cavities are positioned on a first side of a longitudinal slot 100022 and three rows of staple cavities are positioned on a second side of the longitudinal slot 100022. Other arrangements of staple cavities and staples may be possible.

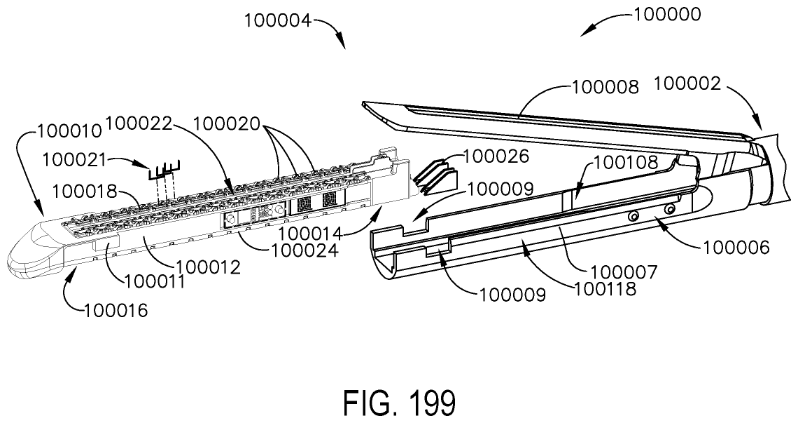

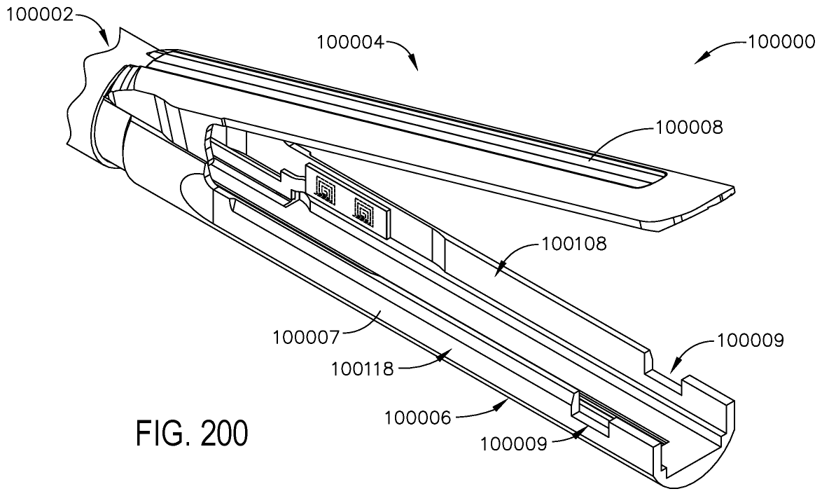

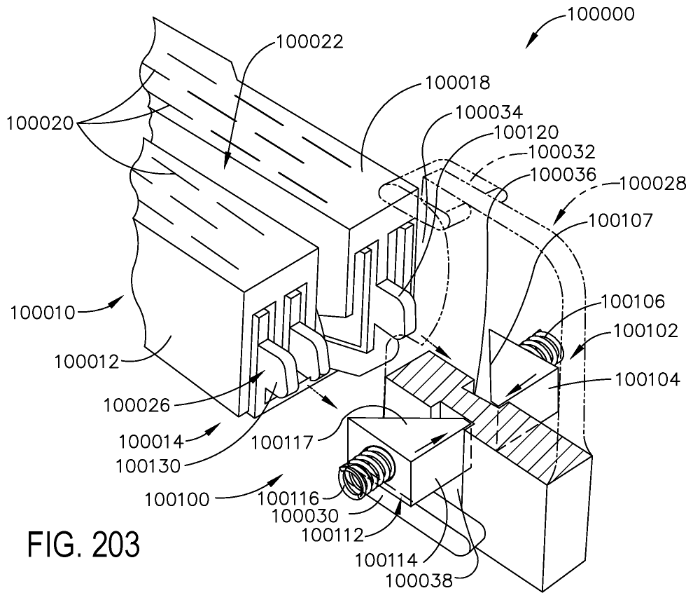

The staples are supported by staple drivers in the cartridge body 100012. Staples supported on staple drivers can be seen in U.S. Patent Application Publication No. 2021/0059672, which is hereby incorporated by reference in its entirety herein. The drivers are movable between a first, or unfired position, and a second, or fired, position to eject the staples from the staple cavities 100020. The drivers are retained in the cartridge body 100012 by a retainer, or pan, 100024 which extends at least partially around the bottom of the cartridge body 100012 and includes resilient members configured to grip the cartridge body 100012 and hold the retainer 100024 to the cartridge body 100012. Various alternative embodiments are envisioned without a retainer, or pan, attached to the cartridge body 100012. The drivers are movable between their unfired positions and their fired positions by a sled 100026 (see FIGS. 199 and 203). The sled 100026 is movable between a proximal position adjacent the proximal end 100014 and a distal position adjacent the distal end 100016. The sled 100026 comprises a plurality of ramped surfaces configured to slide under the drivers and lift the drivers, and the staples supported thereon, toward the anvil 100008. In various embodiments, the staples are not supported by staple drivers, but rather, the staples include integral drive surfaces that are directly engaged by the sled 100026 to lift the staples, examples of which are described in U.S. Patent Application Publication No. 2015/0173756, which is hereby incorporated by reference in its entirety herein.

Further to the above, the sled 100026 is moved distally by a firing actuator 100027 that comprises a firing driver 100028 and a firing bar extending proximally from the firing driver 100028. The firing driver 100028 is configured to contact the sled 100026 and push the sled 100026 toward the distal end 100016. The firing bar comprises a plurality of laminate strips; however, various embodiments are envisioned where the firing bar comprises a rod, for example. The longitudinal slot 100022 defined in the cartridge body 100012 is configured to receive the firing driver 100028 and the firing bar. The anvil 100008 also includes a slot defined therein that receives the firing driver 100028. The firing driver 100028 further comprises a first cam 100030 which engages the first jaw 100006 and a second cam 100032 which engages the second jaw 100008 in the slot of the anvil 100008. As the firing driver 100028 is advanced distally, the first cam 100030 and the second cam 100032 control the distance, or tissue gap, between the deck 100018 of the staple cartridge 100010 and the anvil 100008. The firing driver 100028 also comprises a knife 100034 configured to incise the tissue captured intermediate the staple cartridge 100010 and the anvil 100008. It is desirable for the knife 100034 to be positioned at least partially proximal to the ramped surfaces of the sled 100026 such that the staples are ejected ahead of the knife 100034. In addition, the firing driver 100028 comprises a first notch 100036 defined on a first lateral side thereof and a second notch 100038 defined on a second lateral side thereof.

In various embodiments, the anvil 100008 is moved from the open position to the closed position using a closure system that is controlled separately from the firing driver 100028. The firing driver is part of a firing actuator that is separate and distinctly operable from the closure system. In some such embodiments, the anvil 100008 comprises a ramp on a proximal end thereof and the closure system comprises a closure tube that movable distally to engage the ramp and cam the anvil 100008 to the closed position. In the closed position, the first cam 100030 and the second cam 100032 of the firing driver 100028 translate distally and maintain the anvil 100008 in the closed position. In some embodiments, to transition the anvil 100008 to the open position, the closure tube is retracted proximally and springs positioned within the end effector 100004 bias the anvil 100008 to the open position. In various other embodiments, the anvil 100008 includes a tab and the closure tube defines an aperture which engages the tab as the closure tube moves proximally, thereby positively transitioning the anvil 100008 to the open position. Exemplary closure systems and closure tubes are described in U.S. Patent Application Publication No. 2021/0059672, which was previously incorporated by reference in its entirety herein.

In various other embodiments, the anvil 100008 is moved from the open position to the closed position using the firing driver 100028. In some such embodiments, the anvil 100008 includes a ramp that extends proximally from the slot defined therein that is engaged by the firing driver 100028 during a first portion of the stroke of the firing driver 100028 to move the anvil 100008 to the closed position. In some embodiments, the first portion of the stroke of the firing driver is about 0.100″-0.250″ of the firing driver 100028 travel, for example. After the first portion of the stroke has been completed, the firing driver 100028 can be advanced distally through a second portion of the stroke to deploy staples from the staple cartridge 100010 and incise tissue captured by the end effector, as described above. Firing drivers that close the anvil and fire staples are described in U.S. Pat. No. 11,160,551. In various embodiments, the firing driver 100028 is driven distally by a drive beam extending therefrom. In some such embodiments, the drive beam can comprise a plurality of laminated strips. In various embodiments, the firing driver 100028 is driven distally by an upper drive bar attached at an upper end of the firing driver 100028 and a lower drive bar attached to a lower end of the firing driver 100028, examples of which can be found in U.S. Patent Application Publication No. 2015/0173756, which is hereby incorporated by reference in its entirety herein.

After a staple cartridge 100010 has been fired, or at least partially fired, it can be removed from the elongate channel 100007 and then replaced with another replaceable staple cartridge, if desired. At such point, the surgical stapling system 100000 can be re-used to continue stapling and incising the patient tissue during a surgical procedure. In some instances, however, a previously-fired staple cartridge can be accidentally loaded into the elongate channel 100007. If the firing driver 100028 were to be advanced distally within such a previously-fired staple cartridge, the surgical stapling system 100000 could cut the patient tissue without stapling it, absent more. The surgical stapling system 100000 could similarly cut the patient tissue without stapling it if the firing driver 100028, absent more, were advanced distally through a staple firing stroke without a staple cartridge positioned in the elongate channel 100007 at all. In addition, various surgical staple cartridges may have different arrays of and/or orientations of staples/fasteners therein. The sizes of the staples or fasteners, as well as the number of fasteners, for instance, may vary from cartridge type to cartridge type. To ensure that the staples are properly crimped or formed, it is often the case that surgical staple cartridges are paired with corresponding, compatible anvils that have a proper array of staple-forming pockets defined therein as well as proper cutting and firing components. Should a “non-compatible” cartridge be loaded into a surgical stapling device that has an anvil that is mismatched to the staple cartridge, the staples may not be properly formed during the firing process.

In view of the above, the surgical stapling system 100000 comprises a lockout system 100100 configured to prevent distal the firing driver 100028 from being moved distally unless a compatible and unspent staple cartridge is positioned in the first jaw 100006. The lockout system 100100 includes a first lockout 100102 that includes a first wedge 100104 and a first spring 100106 that biases the first wedge 100104 into the first notch 100036 of the firing driver 100028. The first spring 100106 is mounted to the elongate channel 100007 of the first jaw 100006 on a first lateral side 100108 thereof; however, the first spring 100106 can be mounted in any suitable location. The lockout system 100100 further includes a second lockout 100112 that includes a second wedge 100114 and a second spring 100116 that biases the second wedge 100114 into the second notch 100038 of the firing driver 100028. The second spring 100116 is mounted to the elongate channel 100007 of the first jaw 100006 on a second lateral side 100118 thereof; however, the second spring 100116 can be mounted in any suitable location. Other embodiments are envisioned where the first lockout 100102 and the second lockout 100112 are mounted to the shaft 100002. Also, the springs 100106 and 100116 comprise coil springs, but can comprise any suitable biasing member.

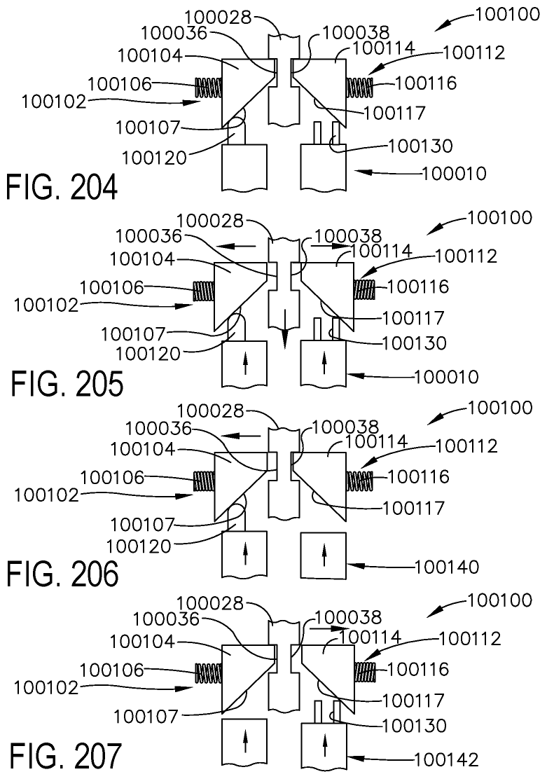

In use, the lockout system 100100 prevents advancement of the firing driver 100028 unless a compatible and unspent staple cartridge is seated in the first jaw 100006. The staple cartridge 100010 includes two discrete authentication keys—a first authentication key 100120 that is configured to transition the first lockout 100102 from a locked state (a state where the first wedge 100104 is positioned in the first notch 100036 of the firing driver 100028, see FIGS. 203 and 204) to an unlocked state (a state where the first wedge 100104 is displaced from the first notch 100036 (i.e. “defeated”) by the first authentication key 100120, see FIG. 205) and a second authentication key 100130 that is configured to transition the second lockout 100112 from a locked state (a state where the second wedge 100114 is positioned in the second notch 100038 of the firing driver 100028, see FIGS. 203 and 204) to an unlocked state (a state where the second wedge 100114 is displaced from the second notch 100038 by the second authentication key 100130 (i.e. “defeated”), see FIG. 205).

The lockouts 100102, 100112 are designed such that a single lockout by itself is able to prevent the firing driver 100028 from moving distally through the staple cartridge 100010. The spring and the wedge of each lockout 100102, 100112 are selected such that the lockout can resist the force being transmitted through the firing driver 100028 and stop the firing driver 100028 from being advanced further distally. As described in greater detail below, the lockout system 100100 ensures that staple cartridge positioned in the first jaw 100006 is both compatible with the stapling instrument 100000 and in an unspent condition in order to permit the firing driver 100028 to be moved through a firing stroke.

As shown in FIG. 203, the staple cartridge 100010 includes a first key 100120 that extends from the proximal end 100014 of the cartridge body 100012. As the staple cartridge 100010 is inserted into the first jaw 100006, the first key 100120 engages a first cam surface 100107 of the first wedge 100104. As the staple cartridge 100010 is seated in the elongate channel 100007, the first key 100120 cams the first wedge 100104 out of the first notch 100036, thus defeating the first lockout 100102 and placing the first lockout 100102 in the unlocked state.

In one aspect, the first lockout 100102 is a compatible staple cartridge lockout as a compatible staple cartridge with a sufficient authentication key, such as the first key 100120 on the cartridge body 100012, transitions the first lockout 100102 to the unlocked position whereas a staple cartridge without the first key 100120, or a sufficient authentication key would not. In various other embodiments, the first key 100120, or a sufficient authentication key is positioned on another portion of the staple cartridge, such as the retainer 100024, for example.

Further to the above, the staple cartridge 100010 includes a second key 100130 that extends from the proximal end of the sled 100026. Notably, as shown in FIG. 203, the sled 100026 is positioned in the proximal, unfired, position, indicating that the staple cartridge 100010 is an unspent staple cartridge (i.e., none of the staples from the staple cartridge have been deployed therefrom). As the staple cartridge 100010 is inserted into the first jaw 100006, the second key 100130 engages a second cam surface 100117 of the second wedge 100114. As the staple cartridge 100010 is seated in the elongate channel 100007, the second key 100130 cams the second wedge 100114 out of the second notch 100038, thus defeating the second lockout 100112 and placing the second lockout 100112 in the unlocked state.

In one aspect, the second lockout 100112 is a spent staple cartridge lockout as a staple cartridge with a sled having a sufficient key in a sufficient location transitions the second lockout to the unlocked position whereas a staple cartridge without a sled having a sufficient key in a sufficient location would not. Should a clinician inadvertently positon a staple cartridge 100010 in the first jaw 100006 that is spent (i.e., all of the staples have been deployed from the staple cartridge), or partially spent (i.e., some, but not all, of the staples have been deployed from the staple cartridge), the second key 100130 would not be able to engage and cam the second wedge 100114 out of the second notch 100038 thereby preventing the firing driver 100028 from advancing distally through the staple cartridge even though the staple cartridge 100010 would unlock the first lockout 100102 with the first key 100120. In various embodiments, the second key 100130 extends a length from the sled 100026 such that the second key 100130 can only transition the second lockout 100112 to the unlocked position if the sled 100026 is in its proximal-most position or a position sufficiently close to the proximal-most position. The sled 100026 is in a sufficiently close position to the proximal-most position where none of the staples have been fired from the staple cartridge 100010 but the sled 100026 has been moved less than a threshold amount from the proximal-most position. In either such position, the sled 100026 is in a proximal unfired position and the second key 100130 would unlock the second lockout 100112 when the staple cartridge 100010 is seated in the first jaw 100006. If, however, the sled 100026 has already been moved distally past the threshold distance when the staple cartridge 100010 is seated in the first jaw 100006, the staple cartridge 100010 would be unable to transition the second lockout 100112 to the unlocked position and, as a result, the staple cartridge would need to be replaced with an unspent (and compatible) staple cartridge in order to fire the stapling instrument 100000. In various embodiments, the second key 100130 can be positioned on another portion of the staple cartridge 100010 that is indicative of the firing status (unspent, spent, partially spent) of the staple cartridge 100010, such as a cover removably positioned over the deck 100018 of the staple cartridge 100010, for example.

FIGS. 204 and 205 illustrate the staple cartridge 100010 being inserted into the first jaw 100006 of the end effector 100004. Notably, the first authentication key 100120 engages the first cam surface 100107 of the first wedge 100104 to drive the first wedge 100104 out of the first notch 100036 and transition the first lockout 100102 to an unlocked state. Similarly, the second authentication key 100130 engages the second cam surface 100117 of the second wedge 100114 to drive the second wedge 100114 out of the second notch 100038 and transition the second lockout 100112 to an unlocked state. With both the first lockout 100102 and the second lockout 100112 in their respective unlocked states, the firing driver 100028 is free to translate through the longitudinal slot 100022 to deploy staples from the staple cartridge 100010 and incise tissue captured by the end effector 100004.

Referring now to FIG. 206, the staple cartridge 100010 has been replaced with a first replacement staple cartridge 100140 that includes a first key 100120 extending therefrom—but does not include a second key. An absent second key can be indicative of, for example, a staple cartridge that does not have a compatible sled even though the sled is in a proximal unspent position or, alternatively, a sled having a sufficient second key not being in an unfired position (i.e., a compatible sled being in at partially spent or spent position). Accordingly, when the first staple cartridge 100140 is removably positioned in the first jaw 100006, the first lockout 100102 is transitioned to the unlocked state (via the first key 100120) but the second lockout 100112 remains in the locked state. In such a state, the lockout system 100100 prevents distal motion of the firing driver 100028. More specifically, in such a state, the second wedge 100114 edges a trailing edge of the second notch 100038 as the firing driver 100028 is moved distally, preventing distal motion of the firing driver 100028.

Referring now to FIG. 207, the staple cartridge 100010 has been replaced with a second replacement staple cartridge 100142 that includes a second key 100130 extending therefrom—but not a first key. An absent first key from the second replacement staple cartridge 100142 can be indicative of the second replacement staple cartridge 100142 being an incompatible staple cartridge. Accordingly, when the second replacement staple cartridge 100142 is seated in the first jaw 100006, the second lockout 100112 is transitioned to the unlocked state (via the second key 100130) but the first lockout 100102 remains in the locked state. In such a state, the lockout system 100100 prevents distal motion of the firing driver 100028. More specifically, in such a state, the first wedge 100104 edges a trailing edge of the first notch 100036 as the firing driver 100028 is moved distally, preventing distal motion of the firing driver 100028.

Accordingly, the foregoing lockout system 100100 provides a “two-factor” authentication lockout which communicates to the clinician that, not only is the staple cartridge positioned in the first jaw 100006 a compatible staple cartridge, but also that the staple cartridge is ready-to-fire (i.e., unspent). This eliminates the possibility of a compatible staple cartridge that has already been spent, or at least partially spent, from being positioned in the first jaw 100006 of the end effector 100004. Furthermore, as outlined above, the lockout system 100100 includes two lockouts 100102, 100112 that each function to restrain the same drive system—the firing driver 100028. The firing driver 100028 is prevented from moving through the staple cartridge to both position the jaws of the end effector 100004 relative to one another and drive staples from the staple cartridge unless both lockouts 100102, 100112 are transitioned to their respective unlocked state via the authentication keys.

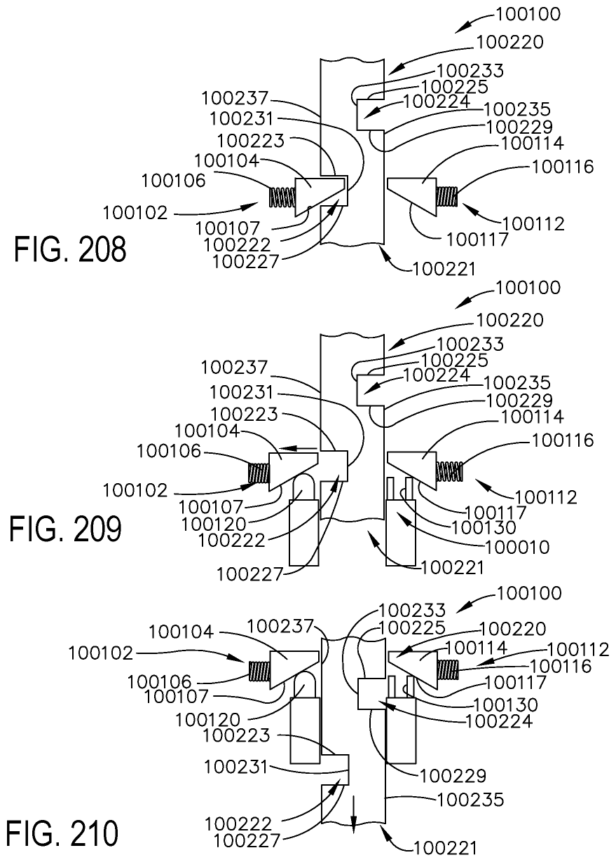

Referring now to FIG. 208, a firing driver 100220 for use with the lockout system 100100 is provided according to at least one aspect of the present disclosure. The firing driver 100220 is similar to the firing driver 100028 in many respects, with some differences noted below. In various embodiments, the firing driver 100220 can be used in lieu of the firing driver 100028 in the firing actuator 100027.

Similar to the firing driver 100028, the firing driver 100220 comprises a first notch 100222 defined on a first lateral side thereof. The first notch 100222 comprises a first leading edge 100227 that is a first longitudinal distance from a distal end 100221 of the firing driver 100220, a first trailing edge 100223 that is a second longitudinal distance from the distal end 100221 of the firing driver 100220, and a first sidewall 100231 that extends between the first leading edge 100227 and the first trailing edge 100223. The distance between the first leading edge 100227 and the first trailing edge 100223 (i.e., the length of the first sidewall 100231) defines a width of the first notch 100222.

In addition, the firing driver 100220 comprises a second notch 100224 defined on a second lateral side thereof. The second notch 100224 comprises a second leading edge 100229 that is a third longitudinal distance from the distal end 100221 of the firing driver 100220, a second trailing edge 100225 that is a fourth longitudinal distance from the distal end 100221 of the firing driver 100220, and a second sidewall 100233 that extends between the second leading edge 100229 and the second trailing edge 100225. The distance between the second leading edge 100229 and the second trailing edge 100225 (i.e., the length of the second sidewall 100233) defines a width of the second notch 100254.

As shown in FIG. 208, the width of the second notch 100224 is the same as the width of the first notch 100222; however, other embodiments are envisioned where the widths are different, as will be described in more detail elsewhere herein. In various embodiments, the second leading edge 100229 and the first leading edge 100227 can be different longitudinal distances from the distal end 100221 of the firing driver 100220; however, other embodiments are envisioned where the leading edges 100227, 100229 are the same distance from the distal end 100221 of the firing driver 100220. Furthermore, as shown in FIG. 208, the second trailing edge 100225 and the first trailing edge 100223 are different longitudinal distances from the distal end 100221 of the firing driver 100220; however, other embodiments are envisioned where the longitudinal distances are the same. For instance, in various embodiments, the first notch 100036 includes a first trailing edge (similar to first trailing edge 100223) and the second notch 100038 includes a second trailing edge (similar to second trailing edge 100225) and both trailing edges are the same distance from the distal end of the firing driver 100028. In some such embodiments, the trailing edges are able to co-operatively apply a resistance force to the firing driver if a user attempts to drive the firing driver though a firing stroke without a compatible unspent staple cartridge positioned in the end effector.

In various embodiments, the firing driver 100220 is usable with the end effector 100004 to both move the anvil 100008 from the open position (FIG. 202) to a closed position (FIG. 201) and then to fire the staples from the staple cartridge 100010. As referenced above, exemplary firing drivers that close the end effector and then fire staples from a staple cartridge are described in U.S. Pat. No. 11,160,551, which is incorporated by reference in its entirety herein.

In operation, the firing driver 100220 is driven through a first motion and a second motion that is distal and subsequent to the first motion. During the first motion, the firing driver 100220 engages a ramp on the second jaw 100008 of the end effector 100004 to move the second jaw 100008 from the open position (FIG. 202) to the closed position (FIG. 201). In various embodiments, the first motion of the firing driver 100220 is about 0.100″-0.250″ of the firing driver 100220 travel, for example. During the second motion subsequent and distal to the first motion, the firing driver 100220 drives the sled 100026 through the staple cartridge 100010 to deploy the staples therefrom. In various embodiments, the second motion of the firing driver 100220 is the remainder of the firing driver 100220 travel after the first motion.

As shown in FIG. 208 and described above, the firing driver 100220 comprises two longitudinally offset notches 100222, 100224. In FIG. 208, the firing driver 100220 is in a proximal position. In the proximal position, the first notch 100222 receives the first wedge 100104, which corresponds to the first lockout 100102 being in the locked state. In addition, in the proximal position, the second wedge 100114 engages a sidewall 100235 of the firing driver 100220, maintaining the second lockout 100112 in the unlocked state. As described elsewhere herein, each of the lockouts 100102, 100112 are designed such that the firing driver 100220 is unable to proceed distally so long as one of the lockouts is in its locked state. Accordingly, as shown in FIG. 208, the firing driver 100220 is unable to proceed distally as the first lockout 100102 is in its locked state. In particular, the first wedge 100104 of the first lockout 100102 engages the first trailing edge 100223 of the first notch 100222, preventing distal motion of the firing driver 100220.

When a compatible and unspent staple cartridge (such as staple cartridge 100010) is inserted into the first jaw 100006 (as shown in FIG. 209), the first authentication key 100120 engages and cams the first wedge 100104 out of the first notch 100222 transitioning the first lockout 100102 to the unlocked state. In addition, the second authentication key 100130 engages the second wedge 100114 to maintain the second lockout 100112 in the unlocked state. Accordingly, the firing driver 100220 is able to proceed distally to move the anvil 100008 to the closed position and fire staples from the staple cartridge 100010, as shown in FIG. 210.

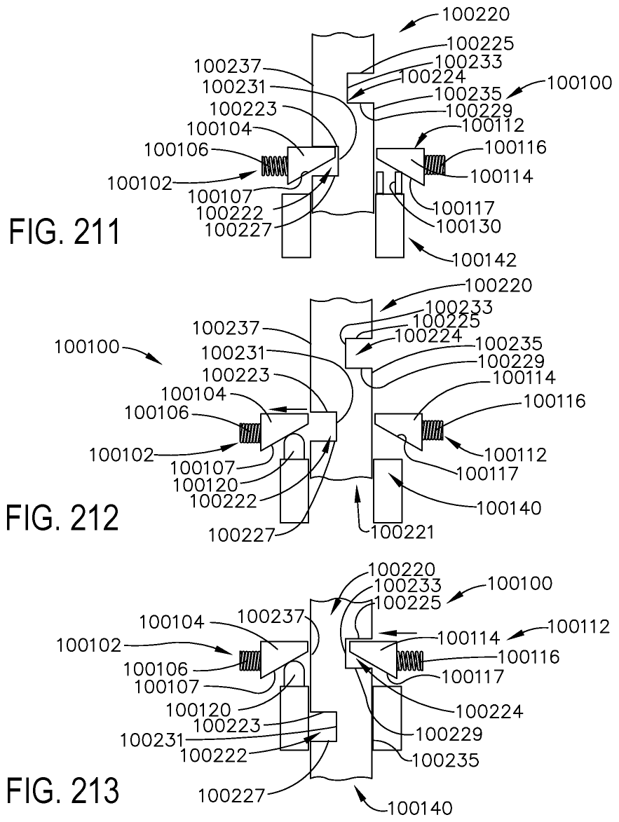

Referring now to FIG. 211, when an incompatible staple cartridge, such as staple cartridge 100142, is inserted into the first jaw 100006, the second authentication key 100130 engages the second wedge 100114 to maintain the second lockout 100112 in the locked state. However, notably, the first staple cartridge 100142 does not include a first authentication key 100120, and therefore, the first lockout 100102 remains in the locked position, preventing distal motion of the firing driver 100220. In particular, the first wedge 100104 of the first lockout 100102 engages the first trailing edge 100223 of the notch 100222 preventing distal motion of the firing driver 100220.

Referring now to FIGS. 212 and 213, when an incompatible staple cartridge, such as staple cartridge 100140, is inserted into the first jaw 100006, the first authentication key 100120 engages and cams the first wedge 100104 out of the first notch 100222, transitioning the first lockout 100102 to the unlocked state. However, notably, the staple cartridge 100140 does not include a second authentication key 100130 to engage and maintain the second lockout 100112 in the unlocked position. However, as referenced above, the second wedge 100114 is engaged with the sidewall 100235 of the firing driver 100220, which holds the second lockout 100112 in the unlocked state. Accordingly, the firing driver 100220 is able to translate distally through a first portion of its firing stroke.

In various embodiments, the first stroke portion comprises the first motion, described above, in which the firing driver 100220 engages a ramp on the second jaw 100008 of the end effector 100004 to move the anvil 100008 from the open position (FIG. 202) to the closed position (FIG. 201). In various embodiments, the first stroke portion comprises about 0.100″-0.250″ of the firing driver 100220 travel, for example. In various embodiments, the first stroke portion is defined as the distance until the second wedge 100114 engages the second trailing edge 100225 of the second notch 100224, described in more detail below. During the first stroke portion, the second wedge 100114 of the second lockout 100112 rides along the sidewall 100235 of the firing driver 100220 and the first wedge 100104 of the first lockout 100102 moves along another sidewall 100237 of the firing driver 100220. During the first motion, the wedge 10114 applies a frictional force to the sidewall 100235 of the firing driver 100220; however, the frictional force is insufficient to prevent distal movement of the firing driver 100220.

Once the firing driver 100220 has translated distally through the first stroke portion, s shown in FIG. 213, the second wedge 100114 becomes aligned with the second notch 100224. As the staple cartridge 100140 does not include a second authentication key 100130 to engage and maintain the second lockout 100112 in the unlocked state, the second spring 100116 biases the second wedge 100114 into the second notch 100224 preventing further distal movement of the firing driver 100220. In particular, the second wedge 100114 of the second lockout 100112 engages the second trailing edge 100225 of the second notch 100224 preventing distal motion of the firing driver 100220.

Accordingly, utilizing the firing driver 100220 with the lockout system 100100 enables to clinician to transition the end effector 100004 between an open state (FIG. 202) and a closed state (FIG. 201) to grasp tissue, for example, when a staple cartridge with a first authentication key 100120 is positioned in the end effector. Such a staple cartridge could comprise a spent staple cartridge 100010, for example. However, owing to the absence of a sufficient second authentication key, the firing driver 100220 is prevented from further advancing through the staple cartridge. The firing driver 100220 allows for a staple cartridge that includes a first authentication key 100120 to maintain some functionality (i.e., allowing for opening and closing of the end effector 100004), but preventing the firing driver 100220 from proceeding through a portion of its stroke that could result in damage to the patient tissue (moving the knife through the staple cartridge that may not include staples therein).

Furthermore, the inclusion of the second cam surface 100117 on the second wedge 100114 enables a clinician to move the firing driver 100220 proximally after the second lockout 100112 has transitioned to the locked state (i.e., when the second wedge 100114 is positioned in the second notch 100224). In particular, as the firing driver 100220 moves proximally, the second cam surface 100117 engages the second leading edge 100229 of the second notch 100224, which cams the second wedge 100114 out of the second notch 100224 and reengages the second wedge 100114 with the sidewall 100235.

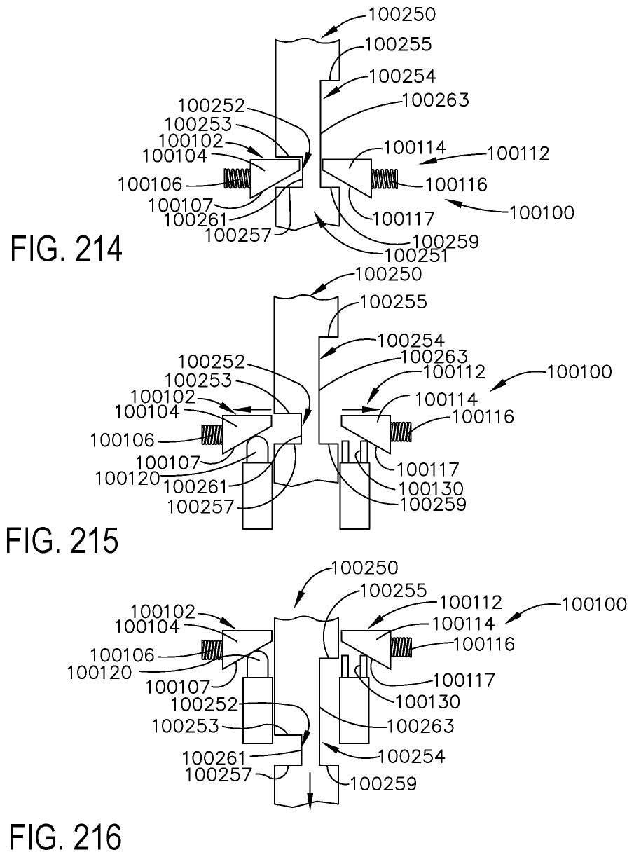

Referring now to FIG. 214, a firing driver 100250 for use with the lockout system 100100 is provided according to at least one aspect of the present disclosure. The firing driver 100250 is similar to the firing driver 100028 and the firing driver 100220 in many respects, with some differences noted below. In various embodiments, the firing driver 100250 can be used in lieu of the firing driver 100028 or firing driver 100250 in the firing actuator 100027.

Similar to the firing driver 100028 and the firing driver 100220, the firing driver 100220 comprises a first notch 100252 defined on a first lateral side thereof. The first notch 100252 comprises a first leading edge 100257 that is a first longitudinal distance from the distal end 100251 of the firing driver 100250, a first trailing edge 100253 that is a second longitudinal distance from the distal end 100251 of the firing driver 100250, and a first sidewall 100261 that extends between the first leading edge 100257 and the first trailing edge 100253. The distance between the first leading edge 100257 and the first trailing edge 100253 (i.e., the length of the first sidewall 100261) defines a width of the first notch 100252.

In addition, the firing driver 100220 comprises a second notch 100254 defined on a second lateral side thereof. The second notch 100254 comprises a second leading edge 100259 that is a third longitudinal distance from the distal end 100251 of the firing driver 100250, a second trailing edge 100255 that is a fourth longitudinal distance from the distal end 100251 of the firing driver 100250, and a second sidewall 100263 that extends between the second leading edge 100259 and the second trailing edge 100255. The distance between the second leading edge 100259 and the second trailing edge 100255 (i.e., the length of the second sidewall 100263) defines a width of the second notch 100254.

As shown in FIG. 214, the width of the second notch 100254 is different than the width of the first notch 100252. In particular, the width of the second notch 100254 is greater than the width of the first notch 100252. In various embodiments, the second leading edge 100259 and the first leading edge 100257 can be the same longitudinal distance from the distal end 100251 of the firing driver 100250; however, other embodiments are envisioned where the leading edges 100257, 100259 are different distances from the distal end 100251 of the firing driver 100250. In various embodiments, the second trailing edge 100255 is a greater longitudinal distance from the distal end 100251 of the firing driver 100250 than the longitudinal distance of the first trailing edge 100253 to the distal end 100251 of the firing driver 100250.

In various embodiments, the firing driver 100250 is usable with the end effector 100004 to both move the anvil 100008 from the open position (FIG. 202) to a closed position (FIG. 201) and then to fire the staples from the staple cartridge 100010. As referenced above, firing drivers that close the end effector and then fire staples from a staple cartridge are described in U.S. Pat. No. 11,160,551, which is incorporated by reference in its entirety herein.

In operation, the firing driver 100250 is driven through a first motion and a second motion that is distal and subsequent to the first motion. During the first motion, the firing driver 100250 engages a ramp on the second jaw 100008 of the end effector 100004 to move the second jaw 100008 from the open position (FIG. 202) to the closed position (FIG. 201). In various embodiments, the first motion of the firing driver 100250 is about 0.100″-0.250″ of the firing driver 100220 travel, for example. During the second motion subsequent and distal to the first motion, the firing driver 100250 drives the sled 100026 through the staple cartridge 100010 to deploy the staples therefrom. In various embodiments, the second motion of the firing driver 100250 is the remainder of the firing driver 100250 travel after the first motion.

As described above, the firing driver 100250 comprises two notches, i.e., notches 100252 and 100254, that comprise different widths. As shown in FIG. 214, the firing driver 100250 is in a proximal position. In the proximal position, the first notch 100252 receives the first wedge 100104, which corresponds to the first lockout 100102 being in the locked state. In addition, the second notch 100254 receives the second wedge 100114, which corresponds to the second lockout 100112 being in the locked state. Unlike the embodiments described elsewhere herein, the firing driver 100250 is capable of translating with one of the lockouts in a locked state, as described in more detail below.

When a compatible and unspent staple cartridge (such as staple cartridge 100010) is inserted into the first jaw 100006 (as shown in FIG. 215), the first authentication key 100120 engages and cams the first wedge 100104 out of the first notch 100252, transitioning the first lockout 100102 to the unlocked state. In addition, the second authentication key 100130 engages and cams the second wedge 100114 out of the second notch 100254, transitioning the second lockout 100112 to the unlocked state. Accordingly, the firing driver 100250 is able to proceed distally to move the anvil 100008 to the closed position and fire staples from the staple cartridge 100010, as shown in FIG. 216.

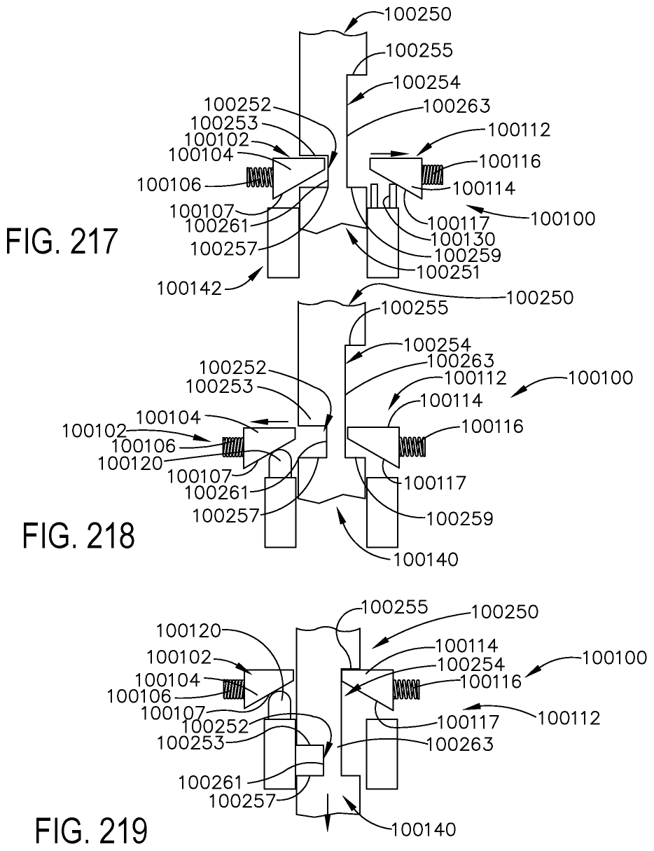

Referring now to FIG. 217, when an incompatible staple cartridge such as staple cartridge 100142 is inserted into the first jaw 100006, the second authentication key 100130 engages and cams the second wedge 100114 out of the second notch 100254 transitioning the second lockout 100112 to the unlocked state. However, notably, the first staple cartridge 100142 does not include a first authentication key 100120 and, therefore, the first lockout 100102 remains in the locked position preventing distal motion of the firing driver 100250. In particular, the first wedge 100104 of the first lockout 100102 engages the first trailing edge 100253 of the first notch 100252 preventing distal motion of the firing driver 100250.

Referring now to FIGS. 218 and 219, when an incompatible staple cartridge such as staple cartridge 100140 is inserted into the first jaw 100006, the first authentication key 100120 engages and cams the first wedge 100104 out of the first notch 100252 transitioning the first lockout 100102 to the unlocked state. However, notably, the staple cartridge 100140 does not include a second authentication key 100130 to engage and cam the second lockout 100112 to the unlocked position. As seen in FIG. 21, the second trailing edge 100255 of the second notch 100254 is positioned proximally with respect to the second wedge 100114, and therefore, the second trailing edge 100255 cannot prevent motion of the firing driver 100250. Accordingly, the firing driver 100250 is able to translate distally through a first portion of its stroke even though the second lockout 100112 is still in a locked condition. During the first portion of the stroke, the wedge 10114 applies a friction force to the second sidewall 100263 of the firing driver 100250; however, the friction force is insufficient to prevent distal movement of the firing driver 100250.

Further to the above, the first stroke portion comprises the first motion, described above, in which the firing driver 100250 engages a ramp on the second jaw 100008 of the end effector 100004 to move the anvil 100008 from the open position (FIG. 202) to the closed position (FIG. 201). In various embodiments, the first stroke portion comprises about 0.100″-0.250″ of the firing driver 100250 travel, for example. In various embodiments, the first stroke portion is defined as the distance between the second wedge 100114 and the second trailing edge 100255, and therefore, the distance that the firing driver 100250 can travel until the second wedge 100114 blocks the second trailing edge 100255 to prevent further displacement of the firing driver 100250, as shown in FIG. 219.

Utilizing the firing driver 100250 with the lockout system 100100 enables to clinician to transition the end effector 100004 between an open state (FIG. 202) and a closed state (FIG. 201) to grasp tissue, for example, when a staple cartridge with a first authentication key is positioned in the end effector. However, absent a sufficient second authentication key, the firing driver 100250 is prevented from further advancing through the staple cartridge. The firing driver 100250 allows for a staple cartridge that includes a first authentication key to maintain some functionality (i.e., allowing for opening and closing of the end effector 100004), but preventing the firing driver 100250 from proceeding through a portion of its stroke that could result in damage to the patient tissue (moving the knife through the staple cartridge that may not include staples therein).

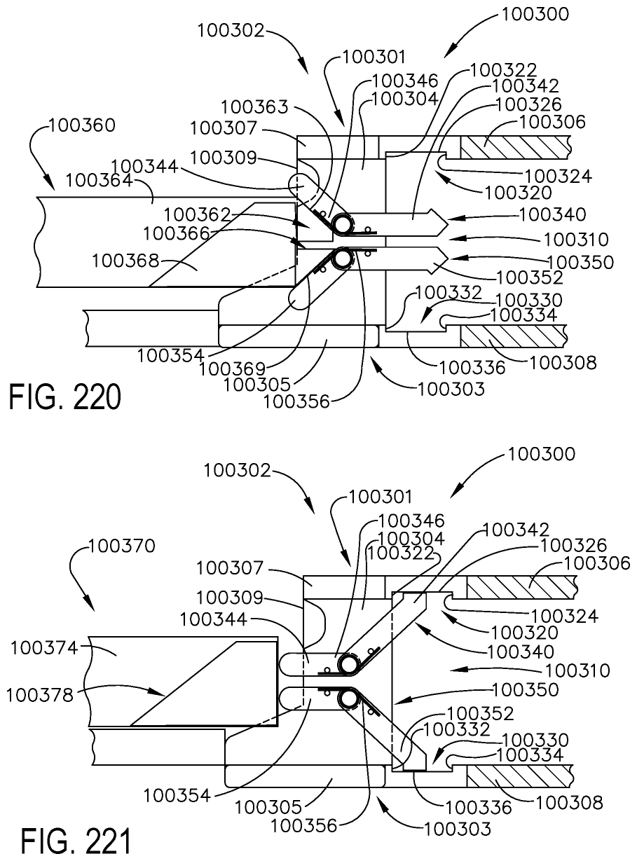

Referring now to FIG. 220, a surgical stapling system 100300 is provided that comprises a firing actuator 100302 and a lockout system 100310. In various embodiments, the firing actuator 100302 and the lockout system 100310 can be used with the end effector 100004. In various embodiments, the firing actuator 100302 and the lockout system 100310 can be used in lieu of the firing driver 100028 and the lockout system 100100 in the surgical stapling system 100000.

The firing actuator 100302 comprises a firing driver 100304, a first push rod 100306 attached at an upper end 100301 of the firing driver 100304, and a second push rod 100308 attached at a lower end 100303 of the firing driver 100304. The first push rod 100306 and the second push rod 100308 can be driven using any suitable drive system, such as a motor drive system, to drive the firing driver 100304 from a proximal position, shown in FIG. 220, to a distal position. An exemplary motor drive system is described in U.S. Patent Application Publication No. 2015/0173756, which is hereby incorporated by reference in its entirety herein. The firing driver 100304 further comprises a first cam 100305 configured to engage a first jaw of an end effector, a second cam 100307 configured to engage a second jaw of the end effector, and a knife 100309 configured to cut tissue after the end effector has been closed. As the firing driver 100304 is advanced distally, the first cam 100305 and the second cam 100307 control the distance, or tissue gap, between the deck of a staple cartridge and an anvil. Driving the firing driver 100304 toward the distal position causes staples to be deployed from a staple cartridge, as described elsewhere herein. In some embodiments, driving the firing driver 100304 distally rotates the anvil toward a closed position (first stroke portion) and then causes staples to be deployed from a staple cartridge (see stroke portion) when the firing driver 100304.

The first push rod 100306 comprises a first notch 100320 defined therein. The first notch 100320 comprises a first leading edge 100322, a first trailing edge 100324, and a first sidewall 100326 extending from the first leading edge 100322 to the first trailing edge 100324. Similarly, the second push rod 100308 comprises a second notch 100330 defined therein. The second notch 100330 comprises a second leading edge 100332, a second trailing edge 100334, and a second sidewall 100336 extending from the second leading edge 100332 to the second trailing edge 100334.

The lockout system 100310 comprises a first lockout 100340 that comprises a first lockout arm 100342, a first cam surface 100344, and a first spring 100346. The first lockout 100340 is pivotable between an unlocked state (a state where the first lockout arm 100342 is rotated within first notch 100320, see FIG. 221) and an unlocked state (a state where the first lockout arm 100342 is rotated out of the first notch 100320, see FIG. 220). The first spring 100346 rotatably biases the first lockout 100340 toward the locked state. The first lockout 100340 is pivotably coupled to an elongate channel of an end effector, such as elongate channel 100007 of end effector 100004, for example, but can be pivotably coupled at any suitable location in the stapling instrument, such as in the shaft, for example.

The lockout system 100310 further comprises a second lockout 100350 that comprises a second lockout arm 100352, a second cam surface 100354, and a second spring 100356. The second lockout 100350 is pivotable between an unlocked state (a state where the second lockout arm 100352 is rotated within the second notch 100330, see FIG. 221) and an unlocked state (a state where the second lockout arm 100352 is rotated out of the second notch 100330, see FIG. 220). The second spring 100356 rotatably biases the second lockout 100350 toward the locked state. The second lockout 100350 is pivotably coupled to an elongate channel of an end effector, such as elongate channel 100007 of end effector 100004, for example, but can be pivotably coupled at any suitable location in the stapling instrument, such as in the shaft, for example. In some embodiments, the first lockout arm 100342 and the second lockout arm 100352 are pivotably coupled to the same lateral side of the elongate channel. In other embodiments, the first lockout arm 100342 and the second lockout arm 100352 are pivotably coupled to different lateral sides of the elongate channel.

In use, the lockout system 100310 prevents advancement of the firing driver 100304 unless a compatible and unspent staple cartridge is positioned in the first jaw of the end effector. Similar to the above, the staple cartridge includes two discrete and separately functioning authentication keys where a first authentication key is configured to transition the first lockout 100340 from the locked state to the unlocked state and a second authentication key is configured to transition the second lockout 100350 from the locked state to the unlocked state, as described in greater detail below.

The first lockout 100340 and the second lockout 100350 are designed such that either one of the lockouts by itself is able to prevent the firing driver 100304 from moving distally through the staple cartridge. The lockout arms and the springs are configured such that the lockouts are able to separately block one of the drive rods of the firing driver 100304. A staple cartridge, such as staple cartridge 100360, for example, includes a first key 100362 that extends from the proximal end of its cartridge body 100364 and includes a first camming surface 100363. As the staple cartridge 100360 is seated in the elongate channel, the first camming surface 100363 engages the first cam surface 100344 to rotate the first lockout 100340 to the unlocked state. With the first lockout 100340 in the unlocked state, the first push rod 100306 is permitted to drive the firing driver 100304 through the staple cartridge 100360 (should the second lockout 100350 also being in the unlocked state, as described in more detail below).

Further to the above, the staple cartridge 100360 includes a second key 100366 that extends from the proximal end of a sled 100368 and includes a second camming surface 100369. Notably, as shown in FIG. 220, the sled 100368 is positioned in a proximal, unfired, position, indicating that the staple cartridge 100360 is an unspent staple cartridge (i.e., none of the staples from the staple cartridge have been deployed therefrom). As the staple cartridge 100360 is removably positioned in the first jaw, the second key 100366 engages the second lockout 100350. As the staple cartridge 100360 is moved proximally to seat the staple cartridge 100360 in the elongate channel, the second camming surface 100369 engages the second cam surface 100354 to rotate the second lockout 100350 to the unlocked state. With the second lockout 100350 in the unlocked state, the second push rod 100308 is permitted to drive the firing driver 100304 through the staple cartridge 100360 (should the first lockout 100340 also being in the unlocked state, as described above).

Further to the above, the first lockout 100340 comprises a compatibility lockout. The second lockout 100350 comprises a spent cartridge lockout but also serves as a compatibility lockout. In order to unlock the second lockout 100350, two conditions regarding the sled of a staple cartridge must be met—the sled must be in a proximal unfired position and the sled must have a sufficient authentication key. In use, should a clinician attempt to drive the firing driver 100304 distally with one or both of the lockouts in their respectively locked position, the lockout arm thereof will engage a corresponding trailing edge of the notch to halt distal advancement of the firing driver 100304. In various embodiments, similar to the above, the first notch 100320 and the second notch 100330 include a width (distance from leading edge to trailing edge) that enables the firing driver 100304 to move through a first stroke portion to move the anvil to the closed position prior to the lockout arms engaging their respective trailing edges to halt the distal translation of the firing driver 100304. Accordingly, the lockout system 100310 would still enable a clinician to transition the end effector between the open position and the closed position even if the installed staple cartridge is not authentic and/or is in a spent condition but not enable the firing driver 100304 to pass through the staple cartridge. Referring now to FIG. 221, the staple cartridge 100360 has been replaced with a first replacement staple cartridge 100370 that includes neither a first key on its cartridge body 100374 nor a second key on its sled 100378. Accordingly, when the first replacement staple cartridge 100370 is seated in the first jaw, both the first lockout 100340 and the second lockout 100350 remain in their respective locked positions but, as described above, the firing driver 100304 can still be moved distally to close the end effector. In alternative embodiments, distal motion of the firing driver 100304 is prevented with one or both of the lockouts in their respective locked position and the end effector cannot be closed by the firing driver 100304 unless both lockouts have been defeated.

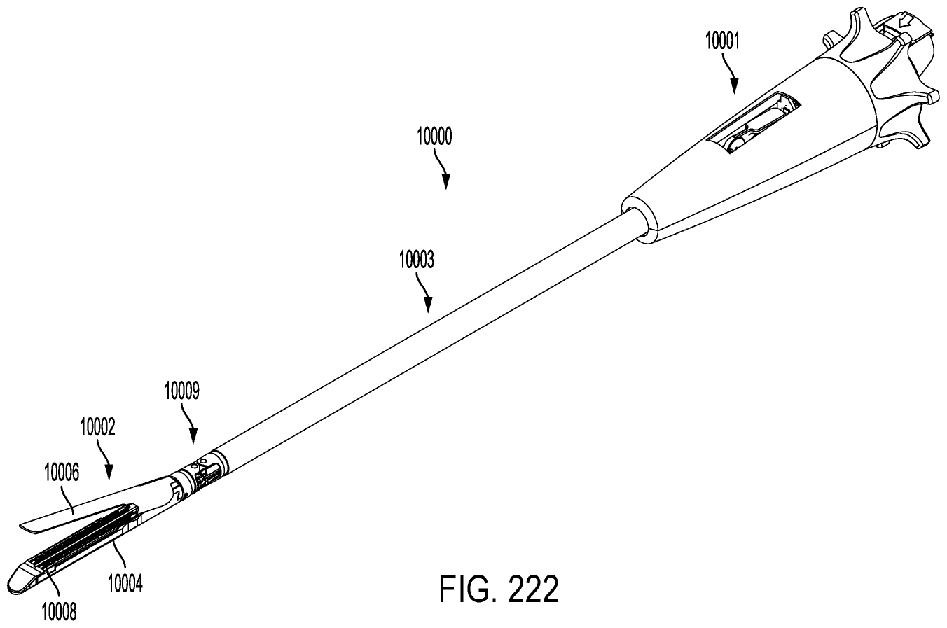

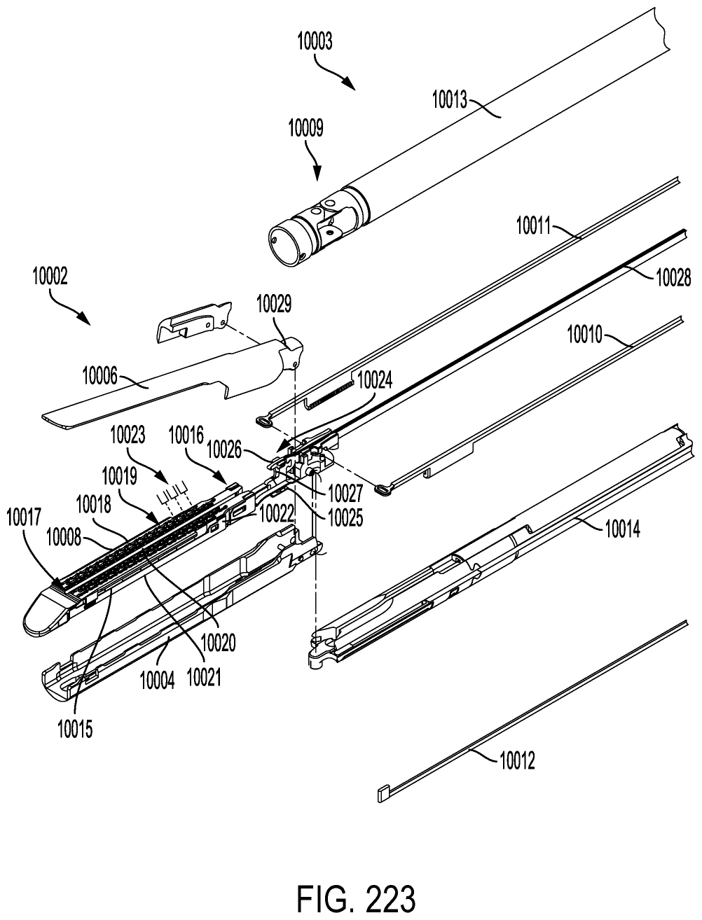

Referring to FIGS. 222 and 223, a surgical stapling system is provided that comprises a shaft assembly 10000 and an end effector 10002 extending from the shaft assembly 10000. The shaft assembly 10000 comprises an attachment portion 10001 and a shaft 10003 extending distally from the attachment portion 10001. The attachment portion 10001 is configured to be attached to a handle of a surgical instrument and/or the arm of a surgical robot, for example.

The end effector 10002 comprises a first jaw 10004 and a second jaw 10006. The first jaw 10004 comprises a staple cartridge 10008. The staple cartridge 10008 is insertable into and removable from the first jaw 10004; however, other embodiments are envisioned in which a staple cartridge is not removable from, or at least readily replaceable from, the first jaw 10004. The second jaw 10006 comprises an anvil configured to deform staples ejected from the staple cartridge 10008. The second jaw 10006 is pivotable relative to the first jaw 10004 between an open position, where the tip of the second jaw 10006 is space apart from the first jaw 10004 (see FIG. 222) and a closed position, where the tip of the second jaw 10006 is adjacent the first jaw 10004; however, other embodiments are envisioned in which the first jaw 10004 is pivotable relative to the second jaw 10006.

The surgical stapling system further comprises an articulation joint 10009 configured to permit the end effector 10002 to be rotated, or articulated, relative to the shaft 10003. The end effector 10002 is rotatable about an articulation axis extending through the articulation joint. Other embodiments are envisioned which do not include an articulation joint 10009. The shaft assembly 10000 comprises cooperating articulation rods 10010, 10011 (e.g., bars) configured to articulate the end effector 10002 relative to the shaft 10003 about the articulation joint 10009. The shaft assembly 10000 further comprises an articulation lock bar 10012 configured to prevent rotation of the end effector 10002, a closure tube 10013 that houses internal components of the shaft assembly 10000, and a spine portion 10014 configured to provide structure support to the closure tube 10013 and shaft assembly 10000.

The staple cartridge 10008 comprises a cartridge body 10015. The cartridge body 10015 includes a proximal end 10016, a distal end 10017, and a deck 10018 extending between the proximal end 10016 and the distal end 10017. In use, the staple cartridge 10008 is positioned on a first side of the tissue to be stapled and the anvil 10006 is positioned on a second side of the tissue. The anvil 10006 is moved toward the staple cartridge 10008 to compress and clamp the tissue against the deck 10018. Thereafter, staples 10023 removably stored in the cartridge body 10015 can be deployed into the tissue. The cartridge body 10015 includes staple cavities 10019 defined therein wherein staples 10023 are removably stored in each of the staple cavities 10019. The staple cavities 10019 are arranged in six longitudinal rows. Three rows of staple cavities are positioned on a first side of a longitudinal slot 10020 and three rows of staple cavities are positioned on a second side of the longitudinal slot 10020. Other arrangements of staple cavities 10019 and staples 10023 may be possible.

The staples 10023 are supported by staple drivers in the cartridge body 10015. Staples supported on staple drivers can be seen in U.S. Patent Application Publication No. 2021/0059672, which is hereby incorporated by reference in its entirety herein. The drivers are movable between a first, or unfired position, and a second, or fired, position to eject the staples 10023 from the staple cavities 10019. The drivers are retained in the cartridge body 10015 by a retainer 10021 which extends around the bottom of the cartridge body 10015 and includes resilient members 10022 configured to grip the cartridge body 10015 and hold the retainer 10021 to the cartridge body 10015. The drivers are movable between their unfired positions and their fired positions by a sled. The sled is movable between a proximal position adjacent the proximal end 10016 and a distal position adjacent the distal end 10017. The sled comprises a plurality of ramped surfaces configured to slide under the drivers and lift the drivers, and the staples 10023 supported thereon, toward the anvil. In various embodiments, the staples are not supported by staple drivers, but rather, the staples include integral drive surfaces that are directly engaged by the sled to lift the staples, examples of which are described in U.S. Patent Application Publication No. 2015/0173756, which is hereby incorporated by reference in its entirety herein.

Further to the above, the sled is moved distally by a firing driver 10024. The firing driver is configured to contact the sled and push the sled toward the distal end 10017. The longitudinal slot 10020 defined in the cartridge body 10015 is configured to receive the firing driver 10024. The anvil 10006 also includes a slot configured to receive the firing driver 10024. The firing driver 10024 further comprises a first cam 10025 which engages the first jaw 10004 and a second cam 10026 which engages the second jaw 10006. As the firing driver 10024 is advanced distally, the first cam 10025 and the second cam 10026 can control the distance, or tissue gap, between the deck 10018 of the staple cartridge 10008 and the anvil 10006. The firing driver 10024 also comprises a knife 10027 configured to incise the tissue captured intermediate the staple cartridge 10008 and the anvil 10006. It is desirable for the knife 10027 to be positioned at least partially proximal to the ramped surfaces such that the staples 10023 are ejected ahead of the knife 10027. The shaft assembly 10000 further comprises a firing bar 10028 that is attached to the firing driver 10024 and that is configured to drive the firing driver through the staple cartridge 10008. In some embodiments, the firing bar 10028 is comprised a plurality of laminated strips. More details of the shaft assembly 10000 can be found in U.S. patent application Ser. No. 15/385,887, entitled METHOD FOR ATTACHING A SHAFT ASSEMBLY TO A SURGICAL INSTRUMENT AND, ALTERNATIVELY, TO A SURGICAL ROBOT, which is incorporated by reference in its entirety.

In various embodiments, the anvil 10006 is moved from the open position to the closed position using a closure system that is controlled separately from the firing driver 10024. In some such embodiments, the firing driver 10024 is considered to be a part of a firing system that is separate and distinctly operable from the closure system. In some such embodiments, the anvil 10006 comprises a ramp 10029 on a proximal end thereof and the closure system comprises a closure driver, such the closure tube 10013, that is movable distally to engage the ramp 10029 and cam the anvil 10006 to the closed position. In the closed position, the first cam 10025 and the second cam 10026 of the firing driver 10024 translate distally and maintain the anvil 10006 in the closed position. In some embodiments, to transition the anvil 10006 to the open position, the closure driver is retracted proximal and springs positioned within the end effector 10002 bias the anvil 10006 to the open position. In various other embodiments, the anvil 10006 includes a tab and the closure driver defines an aperture at the distal end thereof which engages the tab as the closure driver moves proximally, thereby positively transitioning the anvil 10006 to the open position. Exemplary closure systems and closure drivers are described in U.S. Patent Application Publication No. 2021/0059672, which was previously incorporated by reference in its entirety herein.

In various other embodiments, the anvil 10006 is moved from the open position to the closed position using the firing driver 10024. In some such embodiments, the anvil 10006 includes a ramp that extends proximally from the slot defined therein that is engaged by the firing driver 10024 during a first portion of the stroke of the firing driver 10024 to move the anvil 10006 to the closed position. At the end of the first portion of the stroke, the firing driver 10024 can continue advancing distally through a second portion of the stroke to deploy staples from the staple cartridge 10008 and incise tissue captured by the end effector 10002, as described above. Exemplary firing drivers that close the anvil and fire staples are described in U.S. Pat. No. 11,160,551, which was previously incorporated by reference in its entirety herein.

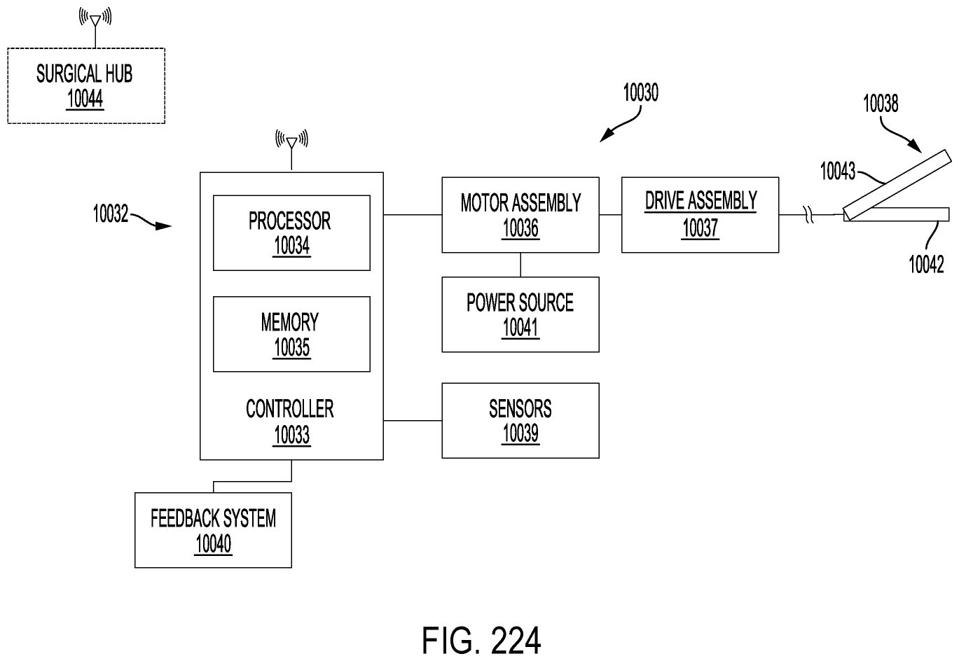

FIG. 224 illustrates a block diagram of a surgical system 10030 for use with one or more surgical instruments, tools, and/or robotic systems in accordance with one or more aspects of the present disclosure. The surgical system 10030 includes a control circuit 10032. The control circuit 10032 includes a controller 10033 comprising a processor 10034 and a storage medium such as, for example, a memory 10035.

A motor assembly 10036 includes one or more motors, driven by motor drivers. The motor assembly 10036 operably couples to a drive assembly 10037 to drive, or effect, one or more motions at an end effector 10038, which can be similar to end effector 10002. The drive assembly 10037 may include any number of components suitable for transmitting motion to the end effector 10038 such as, for example, one or more linkages, bars, tubes, and/or cables, for example. In various embodiments, the drive assembly 10037 can drive a firing driver and/or a closure driver, described herein above.

One or more of sensors 10039, for example, provide real-time feedback to the processor 10034 about one or more operational parameters monitored during a surgical procedure being performed by the surgical system 10030. The operational parameters can be associated with a user performing the surgical procedure, a tissue being treated, and/or one or more components of the surgical system 10030, for example. The sensor 10039 may comprise any suitable sensor, such as, for example, a magnetic sensor, such as a Hall effect sensor, a strain gauge, a pressure sensor, a position sensor, a force sensor, an inductive sensor, such as an eddy current sensor, a resistive sensor, a capacitive sensor, an optical sensor, and/or any other suitable sensor.

Further to the above, in various arrangements, the sensors 10039 may comprise any suitable sensor for detecting one or more conditions at the end effector 10038 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensors 10039 may include one or more sensors located at, or about, an articulation joint, similar to articulation joint 10009, extending proximally from the end effector 10038. Such sensors may include, for example, a potentiometer, a capacitive sensor (slide potentiometer), piezo-resistive film sensor, a pressure sensor, a pressure sensor, or any other suitable sensor type. In some arrangements, the sensor 1938 may comprise a plurality of sensors located in multiple locations in the end effector 1940.

In certain aspects, the system 1930 includes a feedback system 10040 which includes one or more devices for providing a sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The controller 10033 may be programmed to perform various functions such as precise control over the speed and position of the drive assembly 10037. In one aspect, the controller 10033 may be any single-core or multicore processor such as those known under the trade name ARM Cortex by Texas Instruments. In one aspect, the main microcontroller 1933 may be an LM4F230H5QR ARM Cortex-M4F Processor Core, available from Texas Instruments, for example, comprising an on-chip memory of 256 KB single-cycle flash memory, or other non-volatile memory, up to 40 MHZ, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The controller 10033 may be configured to compute a response in the software of the controller 10033. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 10036 includes one or more electric motors and one or more motor drivers. The electric motors can be in the form of a brushed direct current (DC) motor with a gearbox and mechanical links to the drive assembly 10037. In one aspect, a motor driver may be an A3941 available from Allegro Microsystems, Inc.

In various forms, the motor assembly 10036 includes a brushed DC driving motor having a maximum rotational speed of approximately 25,000 RPM. In other arrangements, the motor assembly 10036 may include a brushless motor, a cordless motor, a synchronous motor, a stepper motor, or any other suitable electric motor. The motor driver may comprise an H-bridge driver comprising field-effect transistors (FETs), for example.

The motor assembly 10036 can be powered by a power source 10041. In certain aspects, the power source 10041 includes one or more batteries which may include a number of battery cells connected in series that can be used as the power source to power the motor assembly 10036. In certain circumstances, the battery cells of the power assembly may be replaceable and/or rechargeable. In at least one example, the battery cells can be lithium-ion batteries which can be couplable to and separable from the power assembly.

Further to the above, the end effector 10038 includes a first jaw 10042 and a second jaw 10043. At least one of the first jaw 10042 and the second jaw 10043 is rotatable relative to the other during a closure motion that transitions the end effector 10038 from an open configuration toward a closed configuration. The closure motion may cause the jaws 10042, 10043 to grasp tissue therebetween. In certain arrangements, sensors, such as, for example, a strain gauge or a micro-strain gauge, are configured to measure one or more parameters of the end effector 10038, such as, for example, the amplitude of the strain exerted on the one or both of the jaws 10042, 10043 during a closure motion, which can be indicative of the closure forces applied to the jaws 10042, 10043. The measured strain is converted to a digital signal and provided to the processor 10034, for example. Alternatively, additionally, sensors such as, for example, a load sensor, can measure a closure force and/or a firing force applied to the jaws 10042, 10043. In various embodiments, the sensors can comprise a first sensor that measures a first force on a firing driver, such as firing driver 10024, during a firing stroke and a second sensor that measures a second force on a closure driver, such as the closure tube 10013, during a closure stroke. The processor 10034 can receive these force readings and determine a relationship therebetween, as described in more detail herein below. For instance, the processor 10034 can determine a distribution of force (a ratio) of the force exerted on the firing driver and the closure driver.

In various arrangements, a current sensor can be employed to measure the current drawn by a motor of the motor assembly 10036. The force required to advance the drive assembly 10037 can correspond to the current drawn by the motor, for example. The measured force is converted to a digital signal and provided to the processor 10034.

In one form, strain gauge sensors can be used to measure the force applied to the tissue by the end effector 10038, for example. A strain gauge can be coupled to the end effector 10038 to measure the force on the tissue being treated by the end effector 10038. In one aspect, the strain gauge sensors can measure the amplitude or magnitude of the strain exerted on a jaw of an end effector 10038 during a closure motion which can be indicative of the tissue compression. The measured strain is converted to a digital signal and provided to a processor 10034.

The measurements of the tissue compression, the tissue thickness, and/or the force required to close the end effector on the tissue, as respectively measured by the sensors 10039 can be used by the controller 10033 to characterize the selected position of one or more components of the drive assembly 10037 and/or the corresponding value of the speed of one or more components of the drive assembly 10037. In one instance, a memory (e.g. memory 10035) may store a technique, an equation, and/or a lookup table which can be employed by the microcontroller 1933 in the assessment.

The surgical system 10030 may comprise wired or wireless communication circuits to communicate with surgical hubs (e.g. surgical hub 10044), communication hubs, and/or robotic surgical hubs, for example. Additional details about suitable interactions between a surgical system 10030 and the surgical hub 10044 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is incorporated by reference in its entirety herein.

In various aspects, the control circuit 10032 can be configured to implement various processes described herein. In certain aspects, the control circuit 10032 may comprise a microcontroller comprising one or more processors (e.g., microprocessor, microcontroller) coupled to at least one memory circuit. The memory circuit stores machine-executable instructions that, when executed by the processor, cause the processor to execute machine instructions to implement various processes described herein. The processor may be any one of a number of single-core or multicore processors known in the art. The memory circuit may comprise volatile and non-volatile storage media. The processor may include an instruction processing unit and an arithmetic unit. The instruction processing unit may be configured to receive instructions from the memory circuit of this disclosure.

Alternatively, in certain instances, the control circuit 10032 can be in the form of a combinational logic circuit configured to implement various processes described herein. The combinational logic circuit may comprise a finite state machine comprising a combinational logic configured to receive data, process the data by the combinational logic, and provide an output.

Alternatively, in certain instances, the control circuit 10032 can be in the form of a sequential logic circuit. The sequential logic circuit can be configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In certain instances, the sequential logic circuit may be synchronous or asynchronous. In other instances, the control circuit 10032 may comprise a combination of a processor (e.g., processor 10034) and a finite state machine to implement various processes herein. In other aspects, the finite state machine may comprise a combination of a combinational logic circuit (and the sequential logic circuit, for example.

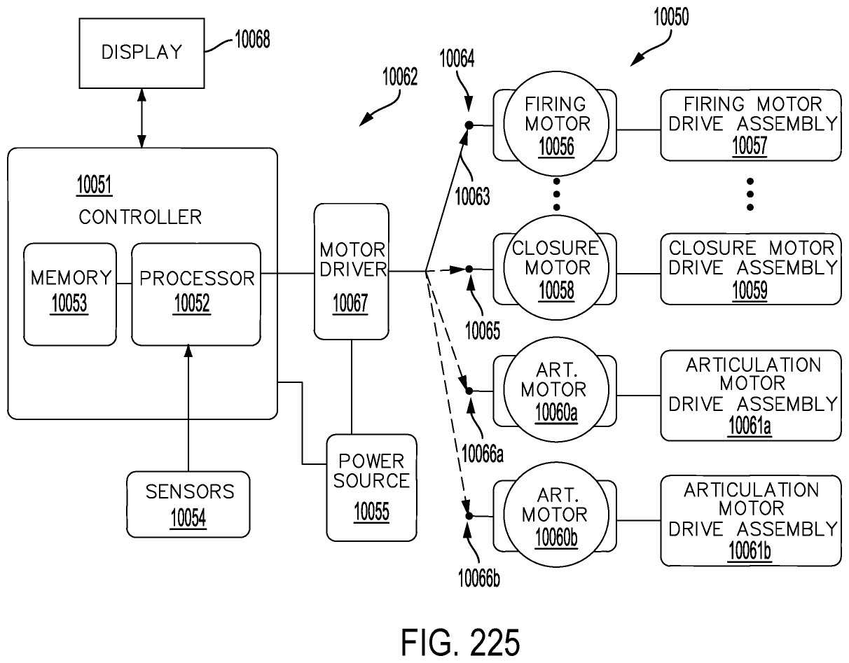

FIG. 225 illustrates a block diagram of a surgical system 10050 for use with one or more surgical instruments, tools, and/or robotic systems in accordance with one or more aspects of the present disclosure. The surgical system 10050 is similar in many respects to the surgical system 10030, which are not repeated herein at the same of detail for brevity. For example, like the surgical system 10030, the surgical system 10050 includes a control circuit comprising a controller 10051 comprising a processor 10052 and a memory 10053, sensors 10054, and a power source 10055, which are similar, respectively, to the controller 10033 (e.g., microcontroller), the processor 10034, the memory 10035, and the power source 10041. Additionally, the surgical system 10050 includes a plurality of motors and corresponding driving assemblies that can be activated to perform various functions.

In certain instances, a first motor can be activated to perform a first function, a second motor can be activated to perform a second function, a third motor can be activated to perform a third function, a fourth motor can be activated to perform a fourth function, and so on. In certain instances, the plurality of motors can be individually activated to cause firing, closure, and/or articulation motions in an end effector, such as end effector 10002 or end effector 10038, as examples. The firing, closure, and/or articulation motions can be transmitted to the end effector through a shaft assembly, such as shaft assembly 10000, for example.

In certain instances, the surgical system 10050 may include a firing motor 10056. The firing motor 10056 may be operably coupled to a firing motor drive assembly 10057 which can be configured to transmit firing motions, generated by the motor 10056 to the end effector, such as displacement of the firing bar 10028 and firing driver 10024. In certain instances, the firing motions generated by the motor 10056 may cause the staples to be deployed from a staple cartridge, such as staple cartridge 10008, into tissue captured by the end effector and/or the knife of a firing driver, such as knife 10027, to be advanced to cut the captured tissue, for example. The firing driver may be retracted by reversing the direction of the motor 10056.

In certain instances, the surgical system 10050 may include a closure motor 10058. The closure motor 10058 may be operably coupled to a closure motor drive assembly 10059 which can be configured to transmit closure motions, generated by the motor 10058 to the end effector, in particular to displace a closure driver, such as outer shaft tube, to close an anvil and compress tissue between the anvil and the staple cartridge. The closure motions may cause the end effector to transition from an open configuration to an approximated, or closed, configuration to grasp tissue, for example. The end effector may be transitioned to an open position by reversing the direction of the motor 10058.

In certain instances, the surgical system 10050 may include one or more articulation motors 10060a, 10060b, for example. The motors 10060a, 10060b may be operably coupled to respective articulation motor drive assemblies 10061a, 10061b, which can be configured to transmit articulation motions generated by the motors 10060a, 10060b to the end effector. In certain instances, the articulation motions may cause the end effector to articulate relative to a shaft, for example. In some embodiments, the first articulation motor 10060a drive a first articulation bar, such as articulation rod 10010, to rotate the end effector in a first direction and the second articulation motor 10060b drive a second articulation bar, such as articulation rod 10011, to rotate the end effector in a second direction opposite the first direction.

As described above, the surgical system 10050 may include a plurality of motors which may be configured to perform various independent functions. In certain instances, the plurality of motors of the surgical instrument or tool can be individually or separately activated to perform one or more functions while the other motors remain inactive. For example, the articulation motors 10060a, 10060b can be activated to cause the end effector to be articulated while the firing motor 10056 remains inactive. Alternatively, the firing motor 10056 can be activated to fire the plurality of staples, and/or to advance the cutting edge, while the articulation motors 10060a, 10060b remains inactive. Furthermore, the closure motor 10058 may be activated simultaneously with the firing motor 10056 to cause the closure driver and the firing driver to advance distally at the same time, or in an overlapping fashion, as described in more detail hereinbelow.

In certain instances, the surgical system 10050 may include a common control module 10062 which can be employed with a plurality of motors of the surgical instrument or tool. In certain instances, the common control module 10062 may accommodate one of the plurality of motors at a time. For example, the common control module 10062 can be couplable to and separable from the plurality of motors of the robotic surgical instrument individually. In certain instances, a plurality of the motors of the surgical instrument or tool may share one or more common control modules such as the common control module 10062. In certain instances, a plurality of motors of the surgical instrument or tool can be individually and selectively engaged with the common control module 10062. In certain instances, the common control module 10062 can be selectively switched from interfacing with one of a plurality of motors of the surgical instrument or tool to interfacing with another one of the plurality of motors of the surgical instrument or tool.

In at least one example, the common control module 10062 can be selectively switched between operable engagement with the articulation motors 10060a, 10060b and operable engagement with either the firing motor 10056 or the closure motor 10058. In at least one example, as illustrated in FIG. 225, a switch 10063 can be moved or transitioned between a plurality of positions and/or states. In a first position 10064, the switch 10063 may electrically couple the common control module 10062 to the firing motor 10056; in a second position 10065, the switch 10063 may electrically couple the common control module 10062 to the closure motor 10058; in a third position 10066a, the switch 10063 may electrically couple the common control module 10062 to the first articulation motor 10060a; and in a fourth position 10066b, the switch 10063 may electrically couple the common control module 10062 to the second articulation motor 10060b, for example. In certain instances, separate common control modules 10062 can be electrically coupled to the firing motor 10056, the closure motor 10058, and the articulations motor 10060a, 10060b at the same time. In certain instances, the switch 10063 may be a mechanical switch, an electromechanical switch, a solid-state switch, or any suitable switching mechanism.

Each of the motors 10056, 10058, 10060a, 10060b may comprise a torque sensor to measure the output torque on the shaft of the motor. The force on an end effector may be sensed in any conventional manner, such as by force sensors on the outer sides of the jaws or by a torque sensor for the motor actuating the jaws.

In various instances, as illustrated in FIG. 225, the common control module 10062 may comprise a motor driver 10067 which may comprise one or more H-Bridge FETs. The motor driver 10067 may modulate the power transmitted from a power source 10055 to a motor coupled to the common control module 10062 based on input from a controller 10051 (e.g., microcontroller)), for example. In certain instances, the controller 10051 can be employed to determine the current drawn by the motor, for example, while the motor is coupled to the common control module 10062, as described above.

In various instances, the processor 10052 may control the motor driver 10067 to control the position, direction of rotation, and/or velocity of a motor that is coupled to the common control module 10062. In certain instances, the processor 10052 can signal the motor driver 10067 to stop and/or disable a motor that is coupled to the common control module 10062.

In certain instances, the memory 10053 may include program instructions for controlling each of the motors of the surgical system 10050 that are couplable to the common control module 10062. For example, the memory 10053 may include program instructions for controlling the firing motor 10056, the closure motor 10058, and the articulation motors 10060a, 10060b. Such program instructions may cause the processor 10052 to control the firing, closure, and articulation functions in accordance with inputs from algorithms or control programs of the surgical instrument or tool.

In certain instances, one or more mechanisms and/or sensors such as, for example, sensors 10054 can be employed to alert the processor 10052 to the program instructions that should be used in a particular setting. For example, the sensors 10054 may alert the processor 10052 to use the program instructions associated with firing, closing, and articulating the end effector. In certain instances, the sensors 10054 may comprise position sensors which can be employed to sense the position of the switch 10063, for example. Accordingly, the processor 10052 may use the program instructions associated with firing the I-beam of the end effector upon detecting, through the sensors 10054 for example, that the switch 10063 is in the first position 10064; the processor 10052 may use the program instructions associated with closing the anvil upon detecting, through the sensors 10054 for example, that the switch 10063 is in the second position 10065; and the processor 10052 may use the program instructions associated with articulating the end effector upon detecting, through the sensors 10054 for example, that the switch 10063 is in the third or fourth position 10066a, 10066b. In various embodiments, the controller 10051 can communicate with a display 10068, which can be similar to feedback system 10040, to provide feedback to a user. In addition, the display 625 can include an input interface such that a user can provide input for controlling the surgical system 10050.

Closure systems that utilize position control closure are plagued by operating in a manner where a closure stoke of a closure driver produces a specific force to tissue captured within the end effector after the end effector has been placed into a closed position. As the tissue thins due to tissue creep and fluid egressing away from the captured tissue, this specific force applied to the tissue diminishes over time. For example, a closure system utilizing position control closure transitions the end effector from an open state toward a closed state by a closure driver which causes a gradual increase in the closure force applied to the tissue. Once the closure driver reaches the end of its closure stroke, the closure force reaches a maximum closure force.

A problem with these closure systems is that, once they have completed their closure stroke, the force applied to the tissue drops over time (due to fluid egress and eventual tissue thinning, for example). As a result of the diminishing closure force after reaching the maximum closure force, the force to drive the firing system reaches a firing force that is greater than the maximum closure force, which places a lot of stress on the firing motor of the firing system.

In one aspect, load control of the closure system enables the closure load, and therefore, the closure force, to stay at an elevated level, improving the pre-firing compression of the tissue, and ultimately, resulting in lower forces to fire. Accordingly, load control allows the surgical system to balance the mechanical firing force impacts between the closure system and the firing system in order to reduce stress on the corresponding drive systems and motors, in particular, the stress on the firing system and the firing motor.

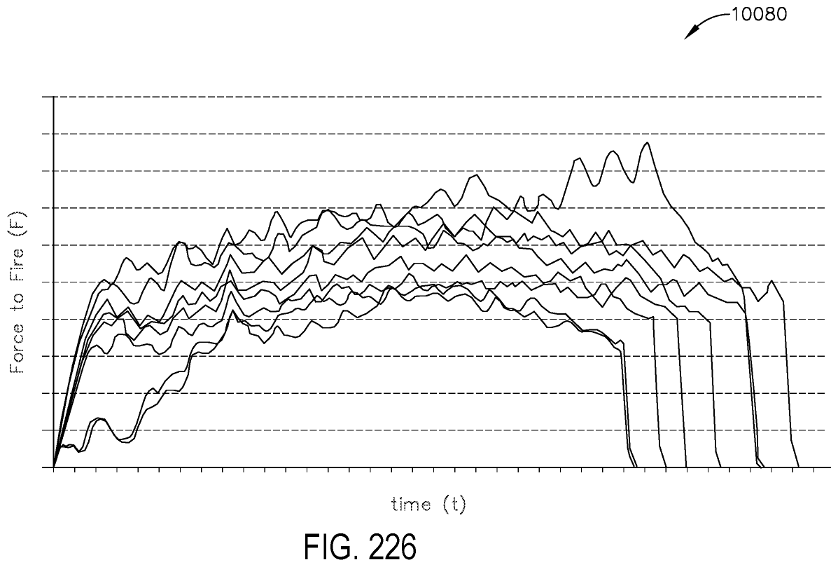

Referring to FIG. 226, a graph 10080 is provided that illustrates the relationship between firing force over time for various firing strokes of firing drivers. As shown, the firing force increases over the course of the firing stroke. The inventors have found that maximization of the closure system can supplement the firing system and enable the firing force to be significantly lowered during a portion of the firing stroke. In some embodiments, the portion of the firing stroke is the first third of the firing stroke. While utilization of the closure system impacts the firing force throughout the entire stroke of the firing driver, the stiffness of the anvil and the deflections of the anvil lends to an outsized impact in the first portion of the stroke over the remaining portion of the stroke. However, in some instances, utilization of the closure system over the entire stroke of the firing system provides the benefit of reducing the force requirement on the firing system, even over the remaining portion of the stroke.

In various embodiments, a surgical system is provided that include a closure system and a firing system. The closure system includes a closure driver, such as closure tube 10013, that is movable from a proximal position toward a distal position during a closure stroke. The closure driver is driven between the proximal position and the distal position by a closure motor, such as closure motor 10058; however, other embodiments are envisioned where the closure driver is driven between the proximal position and the distal position by a manual-drive system that includes a closure trigger manually operable by a clinician. The surgical system further comprises an end effector, such as end effector 10002, that includes a first jaw, such as first jaw 10004, and a second jaw, such as second jaw 10006, that is rotatable relative to the first jaw between an open position and a closed position. In use, the closure driver translates toward the distal position and engages the second jaw, such as on a ramp on the proximal end thereof. The closure driver applies a closure force to the second jaw to rotate the second jaw toward the closed position. In various embodiments, the surgical system further includes a first closure force sensor that senses the closure force applied to the second jaw by the closure driver.

The firing system includes a firing driver, such as firing driver 10024, that is movable from a proximal, unfired position toward a distal, fired position during a firing stroke to deploy staples stored in a staple cartridge, such as staple cartridge 10008, and to incise tissue captured by the end effector with a knife, such as knife 10027. The firing driver is driven between the proximal, unfired position and the distal, fired position by a firing motor, such as firing motor 10056. The firing driver includes a first cam, such as first cam 10025, and a second cam, such as second cam 10026, that engage the first jaw and the second jaw, respectively, during the firing stroke to apply a closure force to the end effector to maintain the second jaw in the closed position. In various embodiments, the surgical system further includes a second closure force sensor that senses the closure force applied to the end effector by the firing driver.

The surgical system further includes a control system, such as controller 10033 or controller 10051, as examples. The control system determines a relationship between the closure system and the firing system, in order to determine which of the closure driver and/or the firing driver the control system should control during the firing stroke of the firing driver, as described in more detail below. In one aspect, determining a relationship between the closure driver and the firing driver comprises determining an amount of co-operation that exists between the closure driver and the firing driver in maintaining the second jaw in the closed position during the firing stroke. In various embodiments, the control system receives data from the first closure force sensor and the second closure force sensor to determine the amount of co-operation that exists between the closure system and the firing system. In various other embodiments, the surgical system further includes various other sensors that sense various other parameters associated with the closure system and the firing system, as described elsewhere herein, that are also used as inputs for determining the amount of co-operation that exists between the closure system and the firing system. For instance, in some embodiments, the control system receives data from position sensors that sense a position of the closure driver and the firing driver and utilize this data to determine an amount of co-operation that exists between the closure system and the firing system.

In various embodiments, the control system determines an amount of co-operation between the closure driver and the firing driver by determining a ratio of the closure force that each of the respective members applies during the firing stroke of the firing driver. In various other embodiments, the amount of co-operation between the closure driver and the firing driver is a ratio of a first parameter of the closure system measured by a first sensor with a second parameter of the firing system measured by a second sensor.

In some embodiments, when the force sensed by the first closure force sensor and the second closure force sensor is identical (a 1-to-1 ratio), the control system determines that a 100% co-operation exists between the closure system and the firing system. This can indicate that the closure motor and the firing motor are experiencing the same, or similar, strains during operation thereof. In some embodiments, when the force sensed by the second force sensor is twice as much as the force sensed by the first force sensor (a 1-to-2 ratio, indicating that the firing driver is applying twice as much closure force as the closure driver), the control system determines that a 50% co-operation exists between the closure system and the firing system. In some embodiments, when the force sensed by the first force sensor is more than the force sensed by the second force sensor (i.e., the closure driver is applying a greater closure force than the firing driver), the control system interprets this a 100% co-operation existing between the closure system and the firing system. Stated another way, the control system recognizes a 100% co-operation between the closure system and the firing system until the closure force applied by the firing driver exceeds the closure force applied by the closure driver.

Based on the amount of co-operation that exists between the closure system and the firing system, the control system makes adjustments to the closure system and/or the firing system. In some embodiments, as described in more detail below, the control system adjusts the firing force threshold of the firing system. In some embodiments, the control system makes adjustments to the closure algorithm that drives the closure driver and/or the firing algorithm that drives the firing driver. This can alleviate strain experienced by each of respective systems, while also lowering the force to fire the firing driver.

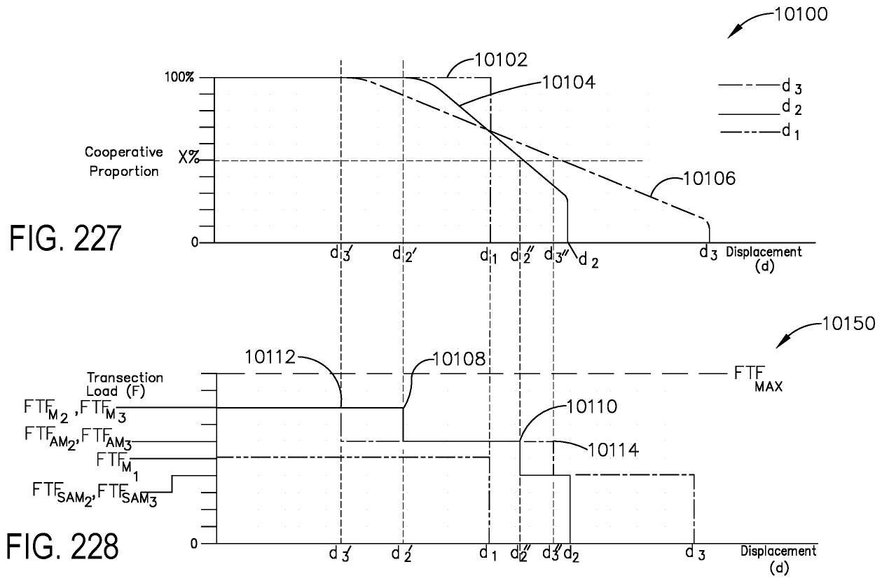

Referring now to FIG. 227, a graph 10100 is provided that illustrates exemplary firing strokes of a firing driver for various sizes of staple cartridges (lengths d₁, d₂, and d₃, where d₂>d₁ and d₃>d₂) and the amount of co-operation that exists between the closure system and the firing system over the firing stroke of the firing driver.

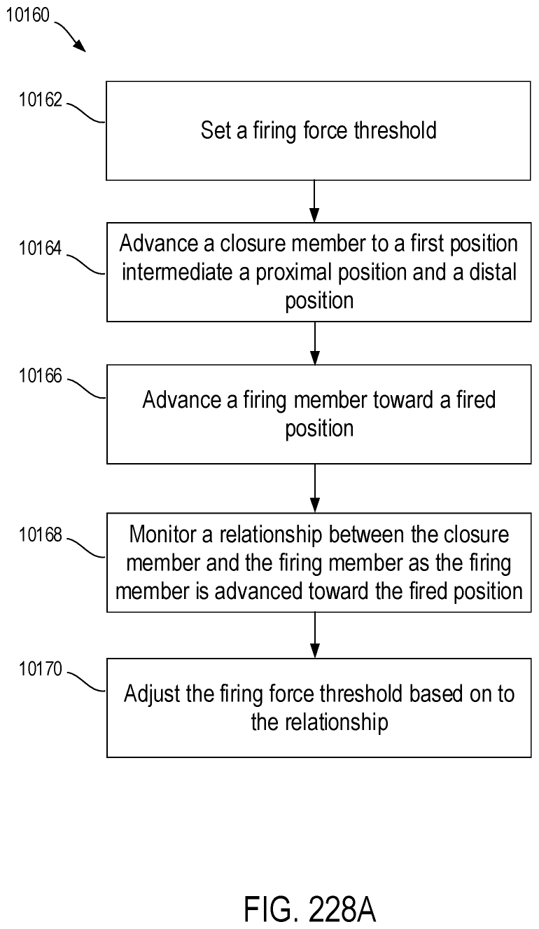

Furthermore, FIG. 228 illustrates a graph 10150 that includes firing force thresholds for each of the respective exemplary staple cartridges over the firing stroke of the firing driver. As seen in FIG. 228, the initial firing force thresholds FTF_{M1} (initial firing force threshold for staple cartridge with length d₁), FTF_{M2} (initial firing force threshold for staple cartridge with length d₂), and FTF_{M3} (initial firing force threshold for staple cartridge with length d₃) are less than a maximum firing force threshold FTF_{MAX} that would result in the firing motor, such as firing motor 10056, stalling. In various other embodiments, the initial firing force threshold for the staple cartridges is at the maximum firing force threshold FTF_{MAX}. In various embodiments, the staple cartridges include an RFID tag positioned thereon and the control system includes an RFID scanner that scans that RFID tag to determine a type of staple cartridge that has been inserted into the end effector. RFID tags and RFID scanners are discussed in more detail in U.S. Patent Application Publication No. 2017/0296173, which is hereby incorporated by reference in its entirety herein. Based on the determination, the control system retrieves the initial firing force threshold stored in a memory, such as memory 10035 or memory 10053. In various other embodiments, the initial firing force threshold is set by a user. For instance, in some embodiments, a user inputs the initial firing force threshold at an input interface, such as at the display 10068.

Based on the amount of co-operation that exists between the closure system and the firing system, the control system adjusts the firing force threshold during the firing stroke of the firing driver. For example, with reference to graphs 10100, 10150, a staple cartridge with a length d₁ is inserted into the first jaw of the end effector. Based on the insertion, an initial firing force threshold FTF_{M1} is set, as discussed above. The end effector is then utilized to grasp tissue within the jaws of the end effector. In particular, the closure driver is advanced distally by the closure motor to apply a closure force to the second jaw to transition the second jaw toward the closed position. In some embodiments, the closure driver is at a first position intermediate the proximal position and the distal position when the second jaw has reached the closed position. In some embodiments, the closure driver halts advancement toward the distal position once a threshold amount of force has been applied to the tissue by the second jaw, thus preventing the tissue from being damaged. In these embodiments, the second jaw is in a position intermediate the open position and the closed position (i.e., a partially closed or partially open position) when the closure driver halts advancement.

Once the closure driver stops advancing, the firing system advances the firing driver through its firing stroke 10102 to deploy staples from the staple cartridge and cut the tissue captured by the end effector. In some embodiments, an amount of time can elapse between the closure driver halting and the firing system initiating to allow for pre-compression of the tissue, which is described in more detail in U.S. patent application Ser. No. 17/957,946, which is hereby incorporated by reference in its entirety herein. As the firing driver is advanced through its firing stroke 10102, the first cam and the second cam of the firing driver apply a closure force to the jaws of the end effector to maintain the end effector in the closed position.

As the firing driver is advanced toward the distal position, the control system monitors an amount of force applied to the end effector by the closure driver (via the first force sensor) and the firing driver (via the second force sensor) and determines an amount of co-operation that exists between the closure system and firing system to maintain the end effector in the closed position. As shown in FIG. 227, the amount of co-operation between the closure system and the firing system is determined by the control system to be at, or substantially at, 100% throughout the entire firing stroke 10102 of the firing driver. With a co-operation of 100%, the firing driver is considered to be in a first firing zone. As the amount of co-operation does not drop below 100%, the control system does not adjust the initial firing force threshold FTF_{M1} during the firing stroke 10102.

As another example, a staple cartridge with a length d₂ is inserted into the first jaw of the end effector. Based on the insertion, an initial firing force threshold FTF_{M2} is set, as discussed above. As seen in graph 10150, the initial firing force threshold FTF_{M2} is greater for the staple cartridge with a length d₂ than the initial firing force threshold FTF_{M1} for the staple cartridge with the length d₁. In some other embodiments, the initial firing force threshold can be the same regardless of the size of the staple cartridge. In some other embodiments, the initial firing force threshold for a staple cartridge having a first length is greater than the initial firing force threshold for a staple cartridge having a second length less than the first length. In some embodiments, the initial firing force threshold is a function of the length of the staple cartridge. In some embodiments, as referenced above, the initial firing force threshold is stored in a memory and retrieved by the control system. In some embodiments, the initial firing force threshold is set by a user.

The end effector is then utilized to grasp tissue within the jaws of the end effector. In particular, the closure driver is advanced distally to apply a closure force to the second jaw to transition the second jaw toward the closed position. In some embodiments, the closure driver is at a first position intermediate the proximal position and the distal position when the second jaw has reached the closed position. In some embodiments, the closure driver halts advancement toward the distal position once a threshold amount of force has been applied to the tissue by the second jaw, thus preventing the tissue from being damaged. In these embodiments, the second jaw is in a position intermediate the open position and the closed position (i.e., a partially closed or partially open position) when the closure driver halts advancement.

Once the closure driver stops advancing, the firing system advances the firing driver through its firing stroke 10104 to deploy staples from the staple cartridge and cut the tissue captured by the end effector. In some embodiments, as described above, an amount of time can elapse between the closure driver halting and the firing system initiating to allow for pre-compression of the tissue. As the firing driver is advanced through its firing stroke 10104, the first cam and the second cam of the firing driver apply a closure force to the jaws of the end effector to maintain the end effector in the closed position.

As the firing driver is advanced toward the distal position, the control system monitors an amount of force applied to the end effector by the closure driver (via the first force sensor) and the firing driver (via the second force sensor) and determines an amount of co-operation that exists between the closure system and the firing system to maintain the end effector in the closed position. As shown in FIG. 227, the amount of co-operation between the closure system and the firing system is determined by the control system to be at, or substantially at, 100% until the firing driver reaches a length d₂′ of its firing stroke 10104. From the initiation of the firing stroke until d₂′, with a co-operation of 100%, the firing driver is considered to be in a first firing zone.

At d₂′, the amount of closure force applied by the firing driver begins to exceed the amount of closure force applied by the closure driver. Therefore, the control system (via the first and second force sensors) determines the amount of co-operation begins to drop below 100%, and therefore, the firing driver transitions to a second firing zone. As discussed above, the control system adjusts the firing force threshold during the firing stroke based on the amount of co-operation that exists between the closure system and the firing system. Accordingly, in the second firing zone, the control system adjusts 10108 the initial firing force threshold FTF_{M2} to a first adjusted firing force threshold FTF_{AM2} that is less than the initial firing force threshold FTF_{M2}. In some embodiments, the first adjusted firing force threshold FTF_{AM2} is stored in a memory and retrieved by the control system. In some embodiments, the first adjusted firing force threshold FTF_{AM2} is user defined. In some embodiments, the firing driver transitions to the second firing zone when the amount of co-operation drops below 100%. In some embodiments, the firing driver transitions to the second firing zone when the amount of co-operation drops a threshold amount below 100% co-operation.

As shown in graph 10100, the firing driver continues to advance through its firing stroke 10104 in the second firing zone and the amount of co-operation continues to diminish. At d₂″, the amount of co-operation reaches a threshold co-operation percentage X %, and therefore, the firing driver transitions to a third firing zone. In some embodiments, the threshold co-operation percentage is 50% co-operation (a 1-to-2 ratio). In some embodiments, the threshold co-operation percentage is 75% (a 3-to-4 ratio). In some other embodiments, the threshold co-operation percentage is 25% (a 1-to-4 ratio). In some embodiments, the threshold co-operation percentage is stored in a memory and retrieved by the control system. In some embodiments, the threshold co-operation percentage is user defined. In some embodiments, the threshold co-operation percentage is set based on the insertion of the staple cartridge into the end effector.

Based on the firing driver transitioning to the third firing zone, the control system adjusts 10110 the first adjusted firing force threshold FTF_{AM2} to a second adjusted firing force threshold FTF_{SAM2} that is less than the first adjusted firing force threshold FTF_{AM2}. In some embodiments, the second adjusted firing force threshold FTF_{SAM2} is stored in a memory and retrieved by the control system. In some embodiments, the second adjusted firing force threshold FTF_{SAM2} is user defined. After the control system adjusts the first adjusted firing force threshold FTF_{AM2} to the second adjusted firing force threshold FTF_{SAM2}, the firing driver continues to advance through the remainder of the firing stroke 10104.

As another example, a staple cartridge with a length d₃ is inserted into the first jaw of the end effector. Based on the insertion, an initial firing force threshold FTF_{M3} is set, as discussed above. As seen in graph 10150, the initial firing force threshold FTF_{M3} is the same as the initial firing force threshold FTF_{M2} for the state cartridge with the length d₂. In some other embodiments, initial firing force threshold FTF_{M3} is greater as the initial firing force threshold FTF_{M2} for the state cartridge with the length d₂. In some embodiments, as referenced above, the initial firing force threshold FTF_{M3} is stored in a memory and retrieved by the control system. In some embodiments, the initial firing force threshold FTF_{M3} is set by a user.

The end effector is then utilized to grasp tissue within the jaws of the end effector. In particular, the closure driver is advanced distally to apply a closure force to the second jaw to transition the second jaw toward the closed position. In some embodiments, the closure driver is at a first position intermediate the proximal position and the distal position when the second jaw has reached the closed position. In some embodiments, the closure driver halts advancement toward the distal position once a threshold amount of force has been applied to the tissue by the second jaw, thus preventing the tissue from being damaged. In these embodiments, the second jaw is in a position intermediate the open position and the closed position (i.e., a partially closed or partially open position) when the closure driver halts advancement.

Once the closure driver stops advancing, the firing system advances the firing driver through its firing stroke 10106 to deploy staples from the staple cartridge and cut the tissue captured by the end effector. In some embodiments, as described above, an amount of time can elapse between the closure driver halting and the firing system initiating to allow for pre-compression of the tissue. As the firing driver is advanced through its firing stroke 10106, the first cam and the second cam of the firing driver apply a closure force to the jaws of the end effector to maintain the end effector in the closed position.

As the firing driver is advanced toward the distal position, the control system monitors an amount of force applied to the end effector by the closure driver (via the first force sensor) and the firing driver (via the second force sensor) and determines an amount of co-operation that exists between the closure system and the firing system to maintain the end effector in the closed position. As shown in graph 10100, the amount of co-operation between the closure system and the firing system is determined by the control system to be at, or substantially at, 100% until the firing driver reaches a length d₃′ of the firing stroke 10106. From the initiation of the firing stroke until d₃′, with a co-operation of 100%, the firing driver is considered to be in a first firing zone.

At d₃′, the amount of closure force applied by the firing driver begins to exceed the amount of closure force applied by the closure driver. Therefore, the control system (via the first and second force sensors) determines the amount of co-operation begins to drop below 100%, and therefore, the firing driver transitions to a second firing zone. As discussed above, the control system adjusts the firing force threshold during the firing stroke based on the amount of co-operation that exists between the closure system and the firing system. Accordingly, in the second firing zone, the control system adjusts 10112 the initial firing force threshold FTF_{M3} to a first adjusted firing force threshold FTF_{AM3} that is less than the initial firing force threshold FTF_{M3}. In some embodiments, the first adjusted firing force threshold FTF_{AM3} is stored in a memory and retrieved by the control system. In some embodiments, the first adjusted firing force threshold FTF_{AM3} is user defined. In some embodiments, the first adjusted firing force threshold FTF_{AM3} is the same as the first adjusted firing force threshold FTF_{AM2}. In some embodiments, the first adjusted firing force threshold FTF_{AM3} is different than the first adjusted firing force threshold FTF_{AM2}. In some embodiments, the firing driver transitions to the second firing zone when the amount of co-operation drops below 100% co-operation. In some embodiments, the firing driver transitions to the second firing zone when the amount of co-operation drops a threshold amount below 100% co-operation.

As shown in graph 10100, the firing driver continues to advance through its firing stroke 10106 in the second firing zone and the amount of co-operation continues to diminish. At d₃″, the amount of co-operation reaches a threshold co-operation percentage X %, and therefore, the firing driver transitions to a third firing zone. In some embodiments, the threshold co-operation percentage is 50% co-operation (a 1-to-2 ratio). In some embodiments, the threshold co-operation percentage is 75% (a 3-to-4 ratio). In some other embodiments, the threshold co-operation percentage is 25% (a 1-to-4 ratio). In some embodiments, the threshold co-operation percentage is stored in a memory and retrieved by the control system. In some embodiments, the threshold co-operation percentage is user defined. In some embodiments, the threshold co-operation percentage is set based on the insertion of the staple cartridge into the end effector.

Based on the firing driver transitioning to the third firing zone, the control system adjusts 10114 the first adjusted firing force threshold FTF_{AM3} to a second adjusted firing force threshold FTF_{SAM3} that is less than the first adjusted firing force threshold FTF_{AM3}. In some embodiments, the second adjusted firing force threshold FTF_{SAM3} is stored in a memory and retrieved by the control system. In some embodiments, the second adjusted firing force threshold FTF_{SAM3} is user defined. After the control system adjusts the first adjusted firing force threshold FTF_{AM3} to the second adjusted firing force threshold FTF_{SAM3}, the firing driver continues to advance through the remainder of the firing stroke.

While the above-provided examples illustrate the control system only making, at most, two adjustments to the firing force threshold, various other embodiments are envisioned where the control system makes additional adjustments to the firing force threshold at various other co-operation percentages. In some embodiments, the control system adjusts the firing force threshold when the amount of co-operation drops below 100% and reaches a first intermediate threshold percentage above the threshold percentage X %, such as 75% when the threshold percentage is 50%. In some embodiments, the control system adjusts the firing force threshold when the amount of co-operation drops below 100% and reaches a first intermediate threshold percentage below the threshold percentage X %, such as 25% when the threshold percentage is 50%.

Referring now to FIG. 228A, a method 10160 for controlling a surgical stapling system is provided, according to at least one aspect of the present disclosure. With reference now to FIG. 228A together with FIGS. 224 and 225, according to one embodiment of the method 10160, in various embodiments, a control system, such as controller 10033 or controller 10051, sets 10162 a firing force threshold based on a staple cartridge being removably positioned in an end effector of the surgical system, as described elsewhere herein. In some embodiments, the firing force threshold is a threshold that would result in a firing motor, such as firing motor 10056, stalling. In some embodiments, the firing force threshold is a threshold that is less than a maximum firing force threshold that would result in the firing motor stalling.

According to one embodiment of the method 10160, the control system advances 10164 a closure driver to a first position intermediate a proximal position and a distal position. In some embodiments, the control system actuates a closure motor, such as closure motor 10058, to advance a closure driver, such as closure tube 10013, distally.

According to one embodiment of the method 10160, the control system advances 10166 a firing driver toward a fired position. In various embodiments, the control system actuates a firing motor, such as firing motor 10056, to advance a firing driver, such as firing driver 10024, distally.

According to one embodiment of the method 10160, the control system monitors 10168 a relationship between the closure driver and the firing driver as the firing driver is advanced toward the fired position. In various embodiments, the control system monitors a relationship between the closure driver and the firing driver by monitoring an amount of co-operation that exists between the closure driver and the firing driver in maintaining an end effector, such as end effector 10002, in a closed position. In some embodiments, as described elsewhere herein, the control system monitors an amount of co-operation that exists by monitoring a ratio of the closure forces that each of the closure driver and the firing driver apply to the end effector.

According to one embodiment of the method 10160, the control system adjusts 10170 the firing force threshold based on to the relationship. In various embodiments, the control system adjusts the firing force threshold based on the amount of co-operation that exists between the firing driver and the closure driver to maintain the end effector in the closure position. In some embodiments, the control system adjusts the firing force threshold to a first adjusted firing force threshold based on the firing driver applying a greater amount of closure force the closure driver. In various embodiments, as described elsewhere herein, the control system is further configured to determine a zone that the firing driver is located based on the relationship between the closure driver and the firing driver. In some such embodiments, as described elsewhere herein, the firing driver is determined to be in a first firing zone based on the control system utilizing the initial firing force threshold for comparison to the measured firing force and the firing driver is determined to be in a second firing zone based on the control system utilizing the first adjusted firing force threshold for comparison to the measured firing force.

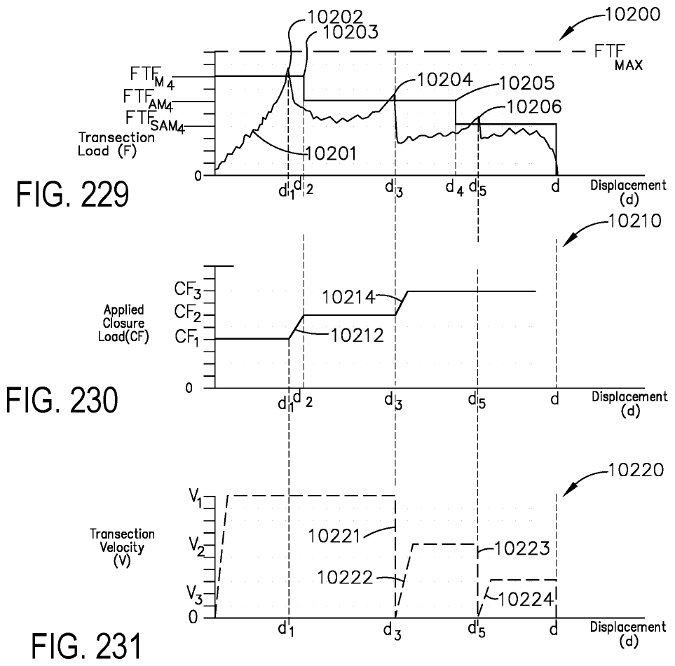

As discussed above, based on the amount of co-operation that exists between the closure system and the firing system, the control system also makes adjustments to the closure system and/or the firing system, as well as makes adjustments to the firing force threshold of the firing system. Referring now to FIGS. 229-231, graphs 10200, 10210, 10220 for an exemplary firing stroke of a staple cartridge with a length d are provided. Graph 10200 illustrates the firing force and firing force thresholds over the firing stroke of the firing driver. Graph 10210 illustrates closure load applied by the closure driver over the firing stroke of the firing driver. Graph 10220 illustrates velocity of the firing driver over the firing stroke of the firing driver.

The staple cartridge is inserted into the first jaw of the end effector. Based on the insertion, an initial firing force threshold FTF_{M4} is set, as discussed above. As seen in graph 10200, the initial firing force threshold FTF_{M4} is less than a firing force threshold FTF_{MAX} that would result in the firing motor stalling. In some embodiments, the initial firing force threshold FTF_{M4} is stored in a memory and retrieved by the control system. In some embodiments, the initial firing force threshold FTF_{M4} is set by a user.

The end effector is then utilized to grasp tissue within the jaws of the end effector. In particular, the closure driver is advanced distally to apply a closure force CF₁ to the second jaw to transition the second jaw toward the closed position. In some embodiments, the closure driver is at a first position intermediate the proximal position and the distal position when the second jaw applies the closure force CF₁ to the second jaw. In some embodiments, the closure driver halts advancement toward the distal position once a threshold amount of force has been applied to the tissue by the second jaw, thus preventing the tissue from being damaged. In these embodiments, the second jaw is in a position intermediate the open position and the closed position (i.e., a partially closed or partially open position) when the closure driver halts advancement.

Once the closure driver stops advancing and is applying the closure force CF₁, the firing system advances the firing driver through its firing stroke at a velocity V₁ (see graph 10220) to deploy staples from the staple cartridge and cut the tissue captured by the end effector. In some embodiments, as described above, an amount of time can elapse between the closure driver halting and the firing system initiating to allow for pre-compression of the tissue. As the firing driver is advanced through its firing stroke, the first cam and the second cam of the firing driver apply a closure force to the jaws of the end effector to maintain the end effector in the closed position.

As the firing driver is advanced toward the distal position, the control system monitors an amount of force applied to the end effector by the closure driver (via a first closure force sensor) and the firing driver (via a second closure force sensor) and determines an amount of co-operation that exists between the closure system and the firing system to maintain the second jaw in the closed position. In addition, the control system monitors the force to fire 10201 the firing driver using a firing force sensor. In some embodiments, the firing force is measured using a force sensor on the firing driver. In some embodiments, the firing force is measured using a current sensor that measures current through the firing motor. Various other sensors for measuring the firing force are described elsewhere herein.

As seen in graph 10200, at d₁ of the firing stroke, the firing force 10201 exceeds 10202 the firing force threshold. At such time, the amount of co-operation between the firing system and the closure system is still determined to be at 100%, and therefore, the firing driver is determined to be in a first firing zone, as discussed elsewhere herein. Based on the firing driver being in the first firing zone, the control system leverages only the closure system and makes an adjustment to the closure algorithm. Namely, the control system causes the closure driver to advance from the first position to a second position without stopping advancement of the firing driver to increase 10212 the applied closure force from CF₁ to CF₂. Based on the increased closure force applied by the closure driver, the firing force 10201 drops below the initial firing force threshold FTF_{M4}. In some embodiments, the second position is proximal to the distal-most position of the closure driver. In some embodiments, the second position is the distal-most position of the closure driver. In some embodiments, the length of the stroke from the first position to the second position is stored in the memory and retrieved by the control system. In some embodiments, the length of the stroke from the first position to the second position is a length that causes the firing force to drop a predetermined amount.

As the firing driver continues to advance through its firing stroke, the control system continues to monitor the force to fire the firing driver and the amount of co-operation between the closure system and the firing system. At d₂, the control system determines that the firing driver begins to apply a greater closure force than the closure driver, and therefore, the co-operation between the closure system and the firing system drops below 100%. Accordingly, the firing driver transitions to a second firing zone. In the second firing zone, the control system adjusts 10203 the initial firing force threshold FTF_{M4} to a first adjusted firing force threshold FTF_{AM4} without pausing advancement of the firing driver.

At d₃ of the firing stroke, the firing force 10201 exceeds 10204 the first adjusted firing force threshold FTF_{AM4}. At such time, the firing driver is still determined to be in second firing zone. Based on the firing driver being in the second firing zone, the control system leverages both the firing system and the control system and makes an adjustment to the firing algorithm and the closure algorithm. Namely, the control system first pauses 10221 advancement of the firing driver, dropping the velocity of the firing driver from V1 to 0, as shown in graph 10220. In addition, the control system causes the closure driver to advance from the second position to a third position to increase 10214 the applied closure force from CF₂ to CF₃. Based on the pause of the firing driver and the increased closure force applied by the closure driver, the firing force 10201 drops below the first adjusted firing force threshold FTF_{AM4}. In some embodiments, the third position is proximal to the distal-most position of the closure driver. In some embodiments, the third position is the distal-most position of the closure driver. In some embodiments, the length of the stroke from the second position to the third position is stored in the memory and retrieved by the control system. In some embodiments, the length of the stroke from the second position to the third position is a length that causes the firing force to drop a predetermined amount.

In various embodiments, the control system causes the firing driver to resume 10222 advancement after a threshold amount of time of being paused. In some embodiments, the threshold amount of time is stored in a memory. In some other embodiments, the threshold amount of time is user defined. In various other embodiments, the control system causes the firing driver to resume advancement after the firing force drops a threshold amount below the first adjusted firing force threshold FTF_{AM4}. At such time, the control system adjusts the firing algorithm such that the firing driver resumes 10222 advancement at a second speed V2 that is less than the first speed V1. In some other embodiments, the control system does not adjust the firing speed of the firing driver. In some embodiments, the second firing speed is stored in a memory and retrieved by the control system. In some embodiments, the second firing speed is user defined. In some embodiments, the second firing speed is a function of the displacement of the firing driver within the firing stroke.

As the firing driver continues to advance through its firing stroke, the control system continues to monitor the force to fire on the firing driver and the amount of co-operation between the closure system and the firing system. At d₄, the control system determines that the co-operation between the closure system and the firing system reaches a threshold co-operation percentage X %, and therefore, the firing driver transitions to a third firing zone. In some embodiments, the threshold co-operation percentage X % is 50% (a 1-to-2 ratio of closure forces applied by the closure driver and firing driver). In some embodiments, the threshold co-operation percentage X % is 75% (a 3-to-4 ratio of closure forces applied by the closure driver and firing driver). In some embodiments, the threshold co-operation percentage X % is 25% (a 1-to-4 ratio of closure forces applied by the closure driver and firing driver). In some embodiments, the threshold co-operation percentage is stored in a memory and retrieved by the control system. In some embodiments, the threshold co-operation percentage is user defined. In some embodiments, the threshold co-operation percentage is set based on the insertion of the staple cartridge into the end effector. Accordingly, based on the transition to the third firing zone, the control system adjusts 10205 the first adjusted firing force threshold FTF_{AM4} to a second adjusted firing force threshold FTF_{SAM4} without pausing advancement of the firing driver.

At d₅ of the firing stroke, the firing force 10201 exceeds 10206 the second adjusted firing force threshold FTF_{SAM4}. At such time, the firing driver is determined to be in the third firing zone. Based on the firing driver being in the third firing zone, the control system only leverages the firing system and makes an adjustment to the firing algorithm. Namely, the control system pauses 10223 advancement of the firing driver, dropping the velocity of the firing driver from V2 to 0, as shown in graph 10220. Unlike in the first firing zone and the second firing zone, the control system does not resume advancement of the closure driver in the third firing zone. Based on the pause of the firing driver, the firing force drops below the second adjusted firing force threshold FTF_{SAM4}.

In various embodiments, the control system causes the firing driver to resume 10224 advancement after a threshold amount of time of being paused. In some embodiments, the threshold amount of time is stored in a memory. In some other embodiments, the threshold amount of time is user defined. In various other embodiments, the control system causes the firing driver to resume 10224 advancement after the firing force drops a threshold amount below the second adjusted firing force threshold FTF_{SAM4}. At such time, the control system adjusts the firing algorithm such that the firing driver resumes advancement 10224 at a third speed V3 that is less than the second speed V2. In some embodiments, the third speed is a minimum speed. In some other embodiments, the control system does not adjust the firing speed of the firing driver. In some embodiments, the third firing speed is stored in a memory and retrieved by the control system. In some embodiments, the third firing speed is user defined. In some embodiments, the third firing speed is a function of the displacement of the firing driver within the firing stroke. After resuming advancement 10224 of the firing driver at the third firing speed, the firing driver reaches the end of its firing stroke at d.

Should the firing driver have exceeded the second adjusted firing force threshold FTF_{SAM4} after resuming advancement 10224 of the firing driver at d₅ and prior to completion of the firing stroke, the control system would again only leverage the firing system to drop the firing force 10201 below the second adjusted firing force threshold FTF_{SAM4}, similar to what was described above.

According, the foregoing provides a control system that leverages the closure system and/or the firing system dependent upon the amount of co-operation that exists between the closure system and the firing system. In one aspect, the amount of leverage that the closure system can provide is greater earlier in the firing stroke of the firing driver than it is later in the firing stroke and, therefore, the closure system is leveraged by the control system exclusively in the first firing zone (when the co-operation is at 100%) and in tandem with the firing system in the second firing zone (when the co-operation is less than 100%, but greater than a threshold percentage X %). After the threshold co-operation percentage has been reached or dropped below, the control system exclusively leverages the firing system for the remainder of the firing stroke. Therefore, the stress on the firing system is diminished as the firing system is exclusively relied upon only in the third zone when the amount of leverage that the closure system can provide is not as great as it is in the first firing zone and the second firing zone.

In addition, in one aspect, the firing zones are variable in length, rather than predefined. For example, the firing driver transitions from the first firing zone to the second firing zone when the co-operation between the closure system and the firing system drops below 100%. When the co-operation drops below 100% is subject to change from one stroke to the next. For instance, the change in co-operation is based on a variety of factors, such as the thickness, the condition, or the type of tissue positioned in the jaws of the end effector. In one instance, the co-operation between the closure system and the firing system may drop below 100% faster with thick tissue positioned between the jaws when compared to thin tissue being positioned between the jaws. Accordingly, the firing zones are subject to change from one firing stroke to the next firing stroke as the transition from one zone to the next relies upon the amount of co-operation between the closure and firing system, which can occur at different times during different firing strokes.

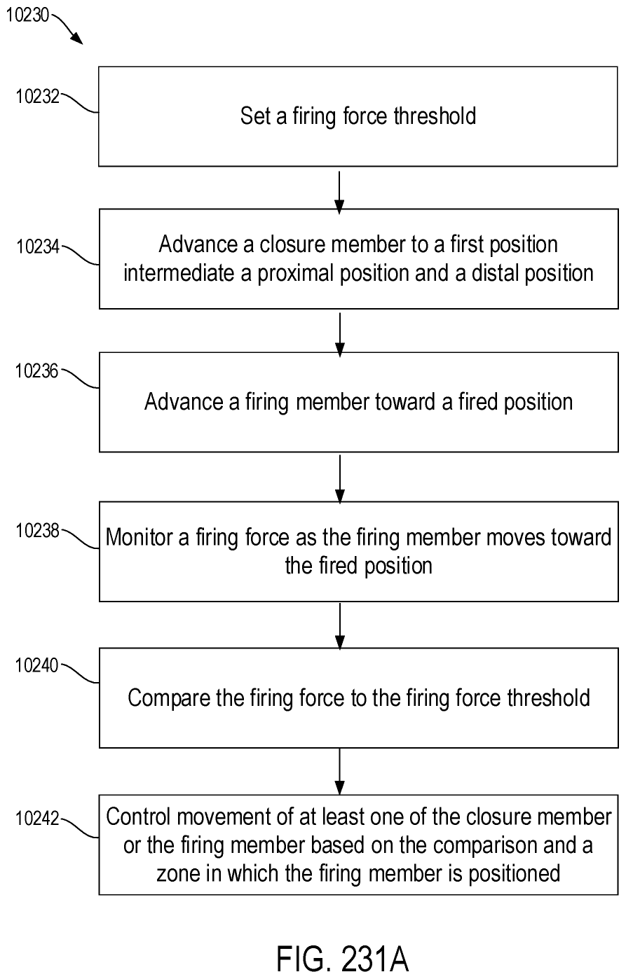

Referring now to FIG. 231A, a method 10230 for controlling a surgical stapling system is provided, according to at least one aspect of the present disclosure. With reference now to FIG. 231A together with FIGS. 224 and 225, according to one embodiment of the method 10230, a control system, such as controller 10033 or controller 10051, sets 10232 a firing force threshold that is a threshold that would result in a firing motor, such as firing motor 10056, stalling. In various embodiments, the control system sets a firing force threshold that is less than a maximum firing force threshold that would result in the firing motor stalling. Various embodiments regarding how the control system sets a firing force threshold are described elsewhere herein.

According to one embodiment of the method 10230, the control system advances 10234 a closure driver to a first position intermediate a proximal position and a distal position. In some embodiments, the control system actuates a closure motor, such as closure motor 10058, to advance a closure driver, such as closure tube 10013, distally.

According to one embodiment of the method 10230, the control system advances 10234 a firing driver toward a fired position. In various embodiments, the control system actuates a firing motor, such as firing motor 10056, which applies a firing force to a firing driver, such as firing driver 10024, to advance the firing driver distally.

According to one embodiment of the method 10230, the control system monitors 10238 the firing force as the firing driver moves toward the fired position. In various embodiments, the surgical stapling system includes a force sensor, such sensor 10039 or sensor 10054, that senses the firing force that the firing motor applies to the firing driver.

According to one embodiment of the method 10230, the control system compares 10240 the firing force to the firing force threshold. In various embodiments, control system is in operation communication with the force sensor and receives the monitored firing force from the force sensor. The control system compares the firing force to the firing force threshold to determine if the firing force is below the firing force threshold or if the firing force has reached or exceeded the firing force threshold.

According to one embodiment of the method 10230, the control system controls 10242 movement of at least one of the closure driver or the firing driver based on the comparison and a zone in which the firing driver is positioned. In various embodiments, based on the control system determining that the firing force has reached or exceeded the firing force threshold, the control system determines which zone that the firing driver is positioned in order to determine which of the closure driver and/or the firing driver should be adjusted, as described elsewhere herein.

In various embodiments, the method 10230 optionally includes monitoring a relationship between the closure driver and the firing driver as the firing driver is advanced toward the fired position. As described elsewhere herein, in various embodiments, the control system monitors the relationship between the closure driver and the firing driver by monitoring an amount of co-operation that exists between the closure driver and the firing driver in maintaining the end effector in its closed state. In various embodiments, the control system monitors an amount of co-operation that exists between the closure driver and the firing driver by monitoring a ratio between closure forces that each of the closure driver and the firing driver apply to the end effector to maintain the end effector in the closed state.

In various embodiments, setting 10232 optionally includes setting an initial firing force threshold, adjusting the initial firing force threshold to a first adjusted firing force threshold based on the monitored relationship between the closure driver and the firing driver, and adjusting the adjusting the first adjusted firing force threshold to a second adjusted firing force threshold based on the monitored relationship between the closure driver and the firing driver. Various embodiments regarding how the control system sets an initial firing force threshold are described elsewhere herein.

In various embodiments, the method 10230 optionally includes determine a zone from a plurality of zones that the firing driver is located. In one aspect, as described elsewhere herein, the control system determines a zone that the firing driver is located according to the amount of co-operation that exists between the closure driver and the firing driver. In some embodiments, with a determined co-operation of 100%, the firing driver is considered to be in a first firing zone. In one aspect, with the firing driver being determined to be in a first firing zone, the method 10230 optionally includes controlling movement of the closure driver and not controlling movement of the firing driver based on the monitored firing force reaching the initial firing force threshold and the firing driver being positioned in the first firing zone.

In some embodiments, with a determined co-operation below 100%, the firing driver is considered to be in a second firing zone. In various embodiments, the control system adjusts the initial firing force threshold to the first adjusted firing force threshold, based on the second closure force being greater than the first closure force, which would result in a co-operation below 100%. In one aspect, with the firing driver being determined to be in the second firing zone, the method 10230 optionally includes controlling movement of the closure driver and the firing driver based on the monitored firing force reaching the first adjusted firing force threshold and the firing driver being positioned in the second firing zone.

In various embodiments, the method 10230 optionally includes set a threshold co-operation percentage. Various embodiments regarding how the control system sets the threshold co-operation percentage are described elsewhere herein. In some embodiments, with a determined co-operation below the threshold co-operation percentage, the firing driver is considered to be in a third firing zone. In various embodiments, the control system adjusts the first adjusted firing force threshold to the second adjusted firing force threshold, based on the second closure force being greater than the first closure force that would result in a co-operation percentage below the threshold co-operation percentage. In one aspect, with the firing driver being determined to be in the third firing zone, the method 10230 optionally includes controlling movement of the firing driver and not controlling movement of the closure driver based on the firing force reaching the second adjusted firing force threshold and the firing driver being positioned in the third firing zone.

As discussed above, it is desirable to maximize the amount of impact that the closure system provides in order to reduce the force to fire the firing driver, and thus, reduce the strain on the firing system. The control system can receive inputs from multiple sensors to provide this outcome. In one aspect, the control system receives tissue response data and device data from the sensors so as to set appropriate parameters for both the closure system and the firing system so as to increase the amount of impact that the closure system provides and reduce the strain on the firing system, while still maintaining a reasonable firing stroke time.

In some embodiments, the tissue response data comprises the thickness of tissue captured by the end effector, the stiffness of tissue captured by the end effector, the condition of the tissue captured by the end effector (heathy, diseased, etc.), the type of tissue captured by the end effector, or any other type of tissue response data described elsewhere herein. In some embodiments, the device data comprises an articulation angle between the end effector and the shaft, the number of uses of the surgical system, the type of staple cartridge positioned in the end effector, or any other type of device data described elsewhere herein. These pieces of data can be used alone, or in combination with each other, by the control system to make any suitable adjustment to the closure system so as to reduce the load on the firing system during the firing stroke.

In various embodiments, a staple cartridge is inserted into the first jaw of the end effector. As described elsewhere herein, the control system interrogates the staple cartridge to determine a type of staple cartridge that is positioned in the end effector. The end effector is then utilized to grasp tissue within the jaws thereof. In particular, the closure driver is advanced from a proximal position toward a distal position to transition the second jaw toward the closed position and apply an initial closure force to the second jaw. In some embodiments, the closure driver is at a position intermediate the proximal position and the distal position when the second jaw applies the closure force to the second jaw. In some embodiments, the closure driver halts advancement toward the distal position once a threshold amount of force has been applied to the tissue by the second jaw, thus preventing the tissue from being damaged. In these embodiments, the second jaw is in a position intermediate the open position and the closed position (i.e., a partially closed or partially open position) when the closure driver halts advancement.

With the tissue grasped, the control system receives data from any number of sensors within the surgical system to determine tissue response data, as described above or elsewhere herein. In addition, the control system receives data from any number of sensors within the surgical system to determine device data, such as the articulation angle of the end effector, the number of uses of the end effector, or any other type of device data described above or elsewhere herein. Based on the tissue response data and the device data, the control system adjusts the closure algorithm such that the closure driver is moved to apply an adjusted closure force to the second jaw. In addition, once the closure tube has stopped moving, the control system provides a message to a user via a display, such as display 10068, indicating that an amount of time should be allowed to elapse prior to firing. This amount of time allows the tissue to relax and stabilize, and thus, will further reduce the firing load on the firing driver. After the amount of time has elapsed, the control system provides a message on the display to the user, indicating to the user that the firing system can be actuated to advance the firing driver through the firing stroke.

As firing system advances the firing driver through its firing stroke to deploy staples from the staple cartridge and cut the tissue captured by the end effector, the control system monitors the firing load on the firing system. In some embodiments, the firing load is measured using a force sensor on the firing driver. In some embodiments, the firing load is measured using a current sensor that measures current through the firing motor. Various other sensors for measuring the firing load on the firing driver and firing system are described elsewhere herein.

In one embodiment, the firing load on the firing driver remains below a threshold firing load during the entire firing stroke, and therefore, no adjustments are made to the firing system or the closure system. In various embodiments, the threshold firing load is stored in a memory and retrievable by the control system. In various other embodiments, the threshold firing load is user defined.

In another embodiment, the control system determines that the firing load reaches or exceeds the threshold firing load. Based on the threshold firing load being reached or exceeded, the control system adjusts the closure algorithm to cause the closure driver to advance distally toward its distal position. By first making adjustments only to the closure system, the control system attempts to reduce firing loads on the firing driver without interrupting the firing stroke of the firing driver, and thus, potentially keeps the firing force below the firing threshold without impacting transection times.

In one aspect, if advancing the closure driver toward the distal position drops the firing load below the threshold firing load, the control system continues to advance the firing driver through its firing stroke without any adjustments to the firing algorithm. In another aspect, if advancing the closure driver toward the distal position does not drop the firing load below the threshold firing load, the control system continues to advance the closure driver until the firing load drops below the firing threshold. The control system continues to advance the closure driver so long as the firing load is above the firing threshold and the closure driver is not at its distal position. Should the closure driver reach its distal position without the firing load dropping below the threshold firing load, the control system then makes adjustments to the firing algorithm to reduce the firing force on the firing system. In some embodiments, adjustments to the firing algorithm comprise pausing advancement of the firing driver. In some embodiments, adjustments to the firing algorithm comprise reducing the speed of the firing driver. In some embodiments, adjustments to the firing algorithm comprise pausing advancement of the firing driver and resuming advancement of the firing driver at a slower speed. Other adjustments to the firing algorithm for reducing firing force are described elsewhere herein.

Accordingly, the control system makes adjustments exclusively to the closure system during the firing stroke of the firing driver until the closure system reaches its maximum limits, such as its distal-most position. This provides the benefit of managing the firing load without impacting the transection time of the firing driver. Once the closure system reaches its maximum limits, the control system switches to making adjustments to the firing system to control the firing loads.

In various embodiments, the firing stroke of the firing driver can include predefined zones and the control system can make adjustments to the closure system and/or firing system based upon which zone the firing driver is within during the firing stroke. In some embodiments, the predefined zones can include a pre-firing zone, a closure firing zone, a middle firing zone, and an end firing zone. In some embodiments, the pre-firing zone comprises a zone when the firing driver is in its proximal position. As described elsewhere herein, in some embodiments, the stapling firing system may not include a closure driver, and instead, the closing of the second jaw is done by the firing driver moving from a proximal position to an intermediate position. As the firing driver moves toward the intermediate position, the cams on the firing driver engage a ramp on the second jaw to cam the second jaw from the open position toward the closed position. In some such embodiments, the closure firing zone is defined between the proximal position of the firing driver and the intermediate position of the firing driver. In some embodiments, the middle firing zone comprises a first portion of the firing stroke, such as the first half of the firing stroke, in which the firing driver deploys staples from the staple cartridge. In some embodiments, the end firing zone comprises a second portion of the firing stroke, such as the remaining half of the firing stroke, in which the firing driver deploys staples from the staple cartridge.

In some embodiments, the zones described elsewhere herein can be defined, or adjusted, based on inputs from sensors within the surgical system to the control system. In some embodiments, the inputs comprise a device type, a tissue type, a tissue location within the end effector, an amount of pressure applied to the tissue, an amount of time that the end effector has clamped the tissue prior to firing, the clamping speed of the second jaw, the force to close (FTC) the second jaw, the voltage applied to the closure motor, the current applied to the closure motor, the length of the staple cartridge positioned in the end effector, the type of cartridge positioned in the end effector, a type of buttress positioned on the deck of the staple cartridge, or the articulation angle of the end effector relative to the shaft, or any combination thereof. In various embodiments, the length of the staple cartridge positioned in the end effector and the type of cartridge positioned in the end effector are used by the control system to set initial firing parameters of the firing system, such as firing speed, firing force, or any other suitable firing parameter described elsewhere herein.

In various embodiments, the surgical stapling system comprises a modular surgical stapling system where the end-effector can be interchanged, examples of which are described in U.S. Patent Application Publication No. 2019/0209249. In one embodiment, the surgical stapling system can include an end effector with a first stapling length and replaced with an end effector with a second stapling length that is different than from the first stapling length. Based on the adjustment, the control system adapts the motor control algorithms for the closure motor and/or firing motor for the specific end effector properties, and then also with the individual calibration properties of the specific device, as discussed above.

As discussed elsewhere herein, a control system can utilize the closure system alone, or in combination with the firing system, during a first portion of the firing stroke when the amount of leverage that the closure system can provide is high. Once the firing driver has transitioned to a second, later portion in the firing stroke, adjustments are only made to the firing algorithm as adjustments to the closure algorithm provide less of an effect than earlier on in the firing stroke.

For instance, as discussed elsewhere herein, the control system determines an amount of co-operation that exists between the closure system and the firing system to define variable zones such that the control system knows whether to adjust the closure algorithm, the firing algorithm, or a combination thereof. In other embodiments, the co-operative zones can be predefined, stored in a memory, such as memory 10035 or memory 10053, and can be retrieved by the control system. In some embodiments, the predefined co-operative zones are a function of the length of the staple cartridge. In some embodiments, the predefined co-operative zones are the same regardless of the staple cartridge length. In some embodiments, the control system defines a high co-operative zone over a first portion of the staple cartridge length and a low co-operative zone over a second portion of the staple cartridge length. In some embodiments, the first portion comprises the first half of the cartridge length and the second portion comprises the second half of the cartridge length. In some embodiments, the first portion comprises the first third of the cartridge length and the second portion comprises the remaining two-thirds of the cartridge length. In some embodiments, the first portion comprises the first two-thirds of the cartridge length and the second portion comprises the remaining third of the cartridge length.

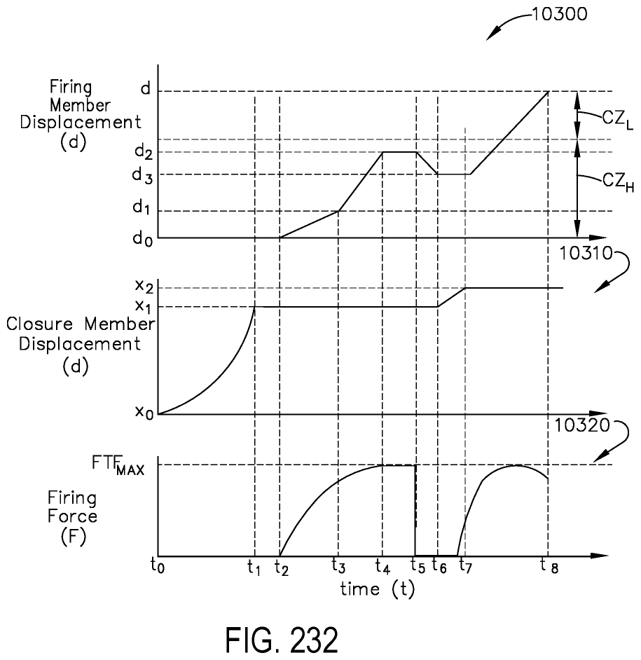

Referring now to FIG. 232, graphs 10300, 10310, 10320 for an exemplary firing stroke of a firing driver though a staple cartridge with a length d are provided. Graph 10300 illustrates the displacement of a firing driver, such as firing driver 10024, over time. Graph 10310 illustrates the displacement of a closure driver, such as closure tube 10013, over time. Graph 10320 illustrates the firing force on the firing driver over time.

The staple cartridge is inserted into the first jaw of the end effector. Based on the insertion, a firing force threshold FTF_{MAX} is set, as shown in graph 10320. In some embodiments, the firing force threshold FTF_{MAX} is defined as the threshold that would result in a firing motor that drives the firing driver, such as firing motor 10056, stalling. In some embodiments, the firing force threshold FTF_{MAX} is stored in a memory and retrievable by the control system. In various embodiments, the staple cartridge includes an RFID tag positioned thereon and the control system includes an RFID scanner that scans that RFID tag to determine a type of staple cartridge that has been inserted into the end effector. Based on the determination, the control system retrieves the firing force threshold FTF_{MAX} stored in a memory. In various other embodiments, the firing force threshold FTF_{MAX} is set by a user.

In addition, based on the insertion of the staple cartridge, the control system defines a high co-operative zone CZ_{H} and a low co-operative zone CZ_{L} for the firing stroke of the firing driver. In various embodiments, the high co-operative zone CZ_{H} and the low co-operative zone CZ_{L} are predefined zones stored in the memory and retrieved by the control system. In various embodiments, the high co-operative zone CZ_{H} and the low co-operative zone CZ_{L} are a function of the staple cartridge length. In various embodiments, the high co-operative zone CZ_{H} and the low co-operative zone CZ_{L} are user defined. In some embodiments, the high co-operative zone CZ_{H} comprises a first portion of the cartridge length and the low co-operative zone CZ_{L} comprises a second portion of the cartridge length. In some embodiments, the high co-operative zone CZ_{H} comprises the first half of the cartridge length and the low co-operative zone CZ_{L} comprises the second half of the cartridge length. In some embodiments, the high co-operative zone CZ_{H} comprises the first third of the cartridge length and the low co-operative zone CZ_{L} comprises the remaining two-thirds of the cartridge length. In some embodiments, the high co-operative zone CZ_{H} comprises the first two-thirds of the cartridge length and the low co-operative zone CZ_{L} comprises the remaining third of the cartridge length.

The end effector is then utilized to grasp tissue within the jaws of the end effector. In particular, referring to graph 10310, from t₀ to t₁, the closure driver is advanced distally from a proximal position x₀ to a first intermediate position x₁ by a closure motor, such as closure motor 10058, to apply a closure force to a second jaw, such as second jaw 10006, to transition the second jaw toward the closed position. In some embodiments, the first intermediate position x₁ is a position intermediate the proximal position x₀ and the distal position of the closure driver when the second jaw has reached the closed position. In some embodiments, the closure driver halts advancement toward the distal position once a threshold amount of force has been applied to the tissue by the second jaw, thus preventing the tissue from being damaged. In these embodiments, the second jaw may be in a position intermediate the open position and the closed position (i.e., a partially closed or partially open position) when the closure driver halts advancement at the first intermediate position x₁.

Once the closure driver stops advancing at t₁, an amount of time is allowed to elapse (t₁ to t₂) before the firing system is initiated. In some embodiments, the amount of time is a predefined amount of time stored in the memory and retrievable by the control system. In some embodiments, the amount of time is variable and a function of the length of the staple cartridge. In some embodiments, the amount of time is a function of the closure force applied to the tissue by the second jaw. For instance, in some embodiments, the amount of time corresponds to the amount of time take for the closure force applied by the second jaw to drop a predefined amount, indicative of tissue thinning. In some embodiments, the amount of time is a function of how long it took the closure driver to reach the first intermediate position x₁. Other amounts of precompression time are described in more detail in U.S. patent application Ser. No. 17/957,946, which is hereby incorporated by reference in its entirety herein.

At t₂, referring to graph 10300 the firing system is actuated to advance the firing driver toward its distal position. In particular, the firing driver is advanced from its proximal positon do through the high co-operative zone CZ_{H}. In various embodiments, the control system comprises a positon sensor that senses a position of the firing driver to determine if the firing driver is within the high co-operative zone CZ_{H} or the low co-operative zone CZ_{L}. From t₂ to t₃, the firing driver is advanced at a first speed from its proximal position do to a lockout position d₁, which causes the firing force to rise (see graph 10320). In various embodiments, the control system includes a force sensor that measures the firing force on the firing driver. In various embodiments, the control system includes a current sensor that senses a current through the firing motor to determine the firing force. Various other force sensors are described elsewhere herein. In some embodiments, at the lockout position, a lockout is situated in the end effector that prevents advancement of the firing driver if a staple cartridge is not positioned therein. An exemplary lockout is provided in U.S. Patent Application Publication No. 2019/0298350, which is hereby incorporated by reference in its entirety herein. From the lockout position d₁, the firing driver is then advanced at a second speed greater than the first speed toward the distal position. Other embodiments are envisioned where the firing driver is advanced at a unitary speed.

At t₄, referring to graph 10320, the control system detects that the firing force has reached the firing force threshold FTF_{MAX} at point d₂ of the firing stroke. Accordingly, the control system pauses advancement of the firing driver for an amount of time (t₄ to t₅). In some embodiments, the amount of time can be predefined and stored in the memory. In some embodiments, the amount of time can be a function of the rate at which the firing force approached the firing force threshold FTF_{MAX}. In various embodiments, the amount of time can be a function of the number of pauses that have occurred in the firing stroke up to the pause. In some embodiments, the length of the pause can be user defined.

As shown in graph 10320, despite the pause in the firing stroke, the firing force does not drop below the firing force threshold FTF_{MAX}, and therefore, the firing driver cannot continue to advance through the firing stroke. However, seen in graph 10300, the control system determines that the firing driver is still within the high co-operative zone CZ_{H}, and therefore, the control system leverages the closure system to reduce the firing force.

As seen in graph 10300, at t₅, the firing driver is retracted proximally by the firing motor from d₂ to d₃, causing the firing force to drop to 0, as seen in graph 10320. In some embodiments, the length of the retraction stroke from d₂ to d₃ is predefined. In some embodiments, the length of the retraction stroke is defined as the displacement of the firing driver required to drop the firing force to 0. At t₆, prior to re-advancing the firing driver, the closure driver is advanced distally from the first intermediate position x₁ to a second intermediate position x₂ to apply additional closure force to the tissue via the second jaw. In some embodiments, the second intermediate position x₂ is proximal to the distal-most position of the closure driver. In some embodiments, the second intermediate position x₂ is the distal-most position of the closure driver. In some embodiments, the length of the stroke from x₁ to x₂ is stored in the memory and retrieved by the control system. In some embodiments, the length of the stroke from x₁ to x₂ is user defined.

At t₇, the control system actuates the firing motor to resume advancement of the firing driver toward its distal position d. As the firing driver advances toward the distal position, the firing force does not exceed the firing force threshold FTF_{MAX}, so the control system takes no additional actions.

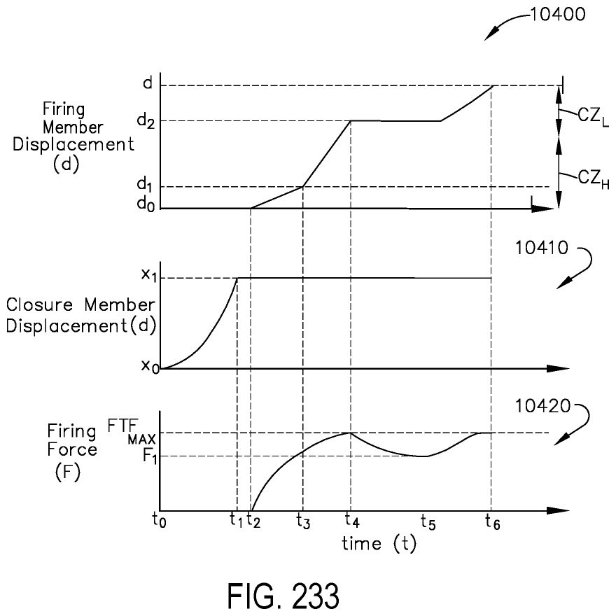

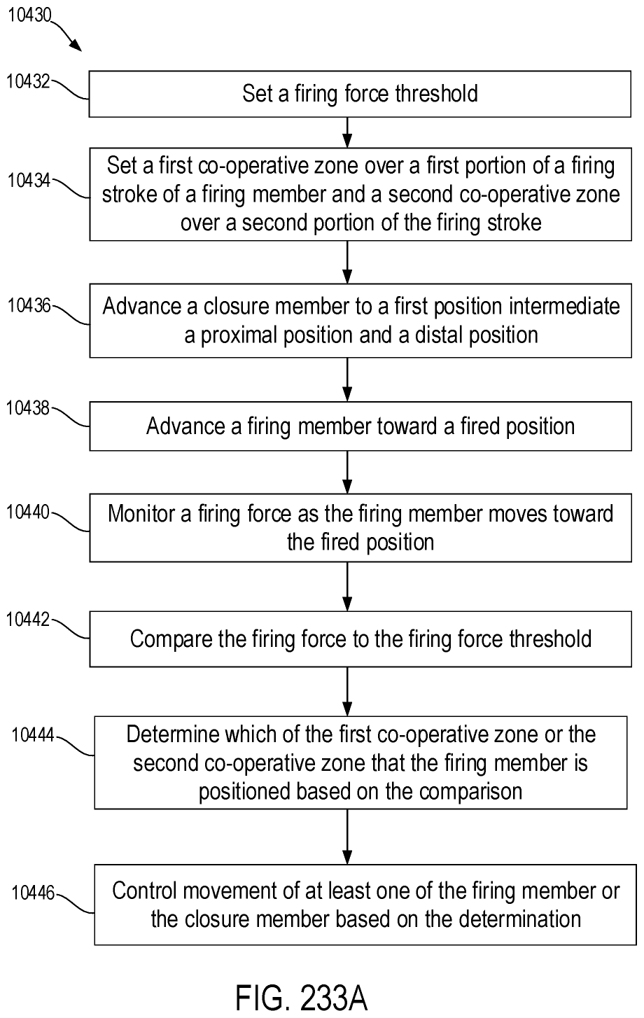

Referring now to FIG. 233, graphs 10400, 10410, 10420 for another exemplary firing stroke of a firing driver though a staple cartridge with a length d are provided. Graph 10400 illustrates the displacement of a firing driver, such as firing driver 10024, over time. Graph 10410 illustrates the displacement of a closure driver, such as closure tube 10013 (e.g., closure driver), over time. Graph 10420 illustrates the firing force on the firing driver over time.

The staple cartridge is inserted into the first jaw of the end effector. Based on the insertion, a firing force threshold FTF_{MAX} is set, as shown in graph 10420. In some embodiments, the firing force threshold FTF_{MAX} is defined as the threshold that would result in a firing motor that drives the firing driver, such as firing motor 10056, stalling. In some embodiments, the firing force threshold FTF_{MAX} is stored in a memory, such as memory 10035 or memory 10053, and retrievable by the control system. In various embodiments, the staple cartridge includes an RFID tag positioned thereon and the control system includes an RFID scanner that scans that RFID tag to determine a type of staple cartridge that has been inserted into the end effector. Based on the determination, the control system retrieves the firing force threshold FTF_{MAX} stored in the memory. In various other embodiments, the firing force threshold FTF_{MAX} is set by a user.

In addition, based on the insertion of the staple cartridge, the control system defines a high co-operative zone CZ_{H} and a low co-operative zone CZ_{L} for the firing stroke of the firing driver. In various embodiments, the high co-operative zone CZ_{H} and the low co-operative zone CZ_{L} are predefined zones stored in the memory and retrieved by the control system. In various embodiments, the high co-operative zone CZ_{H} and the low co-operative zone CZ_{L} are a function of the staple cartridge length. In various embodiments, the high co-operative zone CZ_{H} and the low co-operative zone CZ_{L} are user defined. In some embodiments, the high co-operative zone CZ_{H} comprises a first portion of the cartridge length and the low co-operative zone CZ_{L} comprises a second portion of the cartridge length. In some embodiments, the high co-operative zone CZ_{H} comprises the first half of the cartridge length and the low co-operative zone CZ_{L} comprises the second half of the cartridge length. In some embodiments, the high co-operative zone CZ_{H} comprises the first third of the cartridge length and the low co-operative zone CZ_{L} comprises the remaining two-thirds of the cartridge length. In some embodiments, the high co-operative zone CZ_{H} comprises the first two-thirds of the cartridge length and the low co-operative zone CZ_{L} comprises the remaining third of the cartridge length.

The end effector is then utilized to grasp tissue within the jaws of the end effector. In particular, referring to graph 10410, from t₀ to t₁, the closure driver is advanced distally from a proximal position x₀ to a first intermediate position x₁ by a closure motor, such as closure motor 10058, to apply a closure force to a second jaw, such as second jaw 10006, to transition the second jaw toward the closed position. In some embodiments, the first intermediate position x₁ is a position intermediate the proximal position x₀ and the distal position of the closure driver when the second jaw has reached the closed position. In some embodiments, the closure driver halts advancement toward the distal position once a threshold amount of force has been applied to the tissue by the second jaw, thus preventing the tissue from being damaged. In these embodiments, the second jaw may be in a position intermediate the open position and the closed position (i.e., a partially closed or partially open position) when the closure driver halts advancement at the first intermediate position x₁.

Once the closure driver stops advancing at t₁, an amount of time is allowed to elapse (from t₁ to t₂) before the firing system is initiated. In some embodiments, the amount of time is a predefined amount of time stored in the memory and retrievable by the control system. In some embodiments, the amount of time is variable and a function of the length of the staple cartridge. In some embodiments, the amount of time is a function of the closure force applied to the tissue by the second jaw. For instance, in some embodiments, the amount of time corresponds to the amount of time take for the closure force applied by the second jaw to drop a predefined amount, indicative of tissue thinning. In some embodiments, the amount of time is a function of how long it took the closure driver to reach the first intermediate position x₁. Other amounts of precompression time are described in more detail in U.S. patent application Ser. No. 17/957,946, which is hereby incorporated by reference in its entirety herein.

At t₂, referring to graph 10400, the firing system is actuated to advance the firing driver toward its distal position. In particular, the firing driver is advanced from its proximal positon do through the high co-operative zone CZ_{H}. In various embodiments, the control system comprises a positon sensor that senses a position of the firing driver to determine if the firing driver is within the high co-operative zone CZ_{H} or the low co-operative zone CZ_{L}. From t₂ to t₃, the firing driver is advanced at a first speed from its proximal position do to a lockout position d₁, which causes the firing force to rise (see graph 10420). In various embodiments, the control system includes a force sensor that measures the firing force on the firing driver. In various embodiments, the control system includes a current sensor that senses a current through the firing motor to determine the firing force. In some embodiments, at the lockout position d₁, a lockout is situated in the end effector that prevents advancement of the firing driver if a staple cartridge is not positioned therein, as discussed herein above. From the lockout position d₁, the firing driver is then advanced at a second speed greater than the first speed toward the distal position. Other embodiments are envisioned where the firing driver is advanced at a unitary speed.

At t₄, referring to graph 10420, the control system detects that the firing force has reached the firing force threshold FTF_{MAX} at point d₂ of the firing stroke. Accordingly, the control system pauses advancement of the firing driver, which causes the firing force to drop below the firing force threshold FTF_{MAX}.

As shown in graph 10400, unlike the example shown in graph 10300, the control system determines that the firing driver is no longer within the high co-operative zone CZ_{H}, but rather, has moved into the low co-operative zone CZ_{L}. Therefore, the control system does not leverage the closure system to reduce the firing force. Rather, in the low co-operative zone CZ_{L}, the control system controls the firing load on the firing driver by making adjustments to the firing algorithm. In some embodiments, the control system adjusts the firing algorithm to pause advancement of the firing driver for an amount of time. In some embodiments, the amount of time can be predefined, stored in the memory, and retrieved by the control system. In some embodiments, the amount of time is a function of the rate at which the firing force approached the firing force threshold FTF_{MAX}. In various embodiments, the amount of time is a function of the number of pauses that have occurred in the firing stroke. In some embodiments, the length of the pause is user defined. In some embodiments, the amount of time is an amount of time taken for the firing force to drop a threshold amount below the firing force threshold FTF_{MAX}. In some embodiments, the control system adjusts the firing algorithm by reducing the speed of the firing driver. In some embodiments, the control system adjusts the firing algorithm by reducing the speed of the firing driver after pausing the firing driver. In some embodiments, the control system adjusts the firing algorithm by reducing the speed of the firing driver to a minimum speed. In some embodiments, the control system adjusts the firing algorithm by reducing the speed of the firing driver to a speed intermediate a current speed and the minimum speed.

As seen in graphs 10400 and 10420, at t₅, the firing driver resumes advancement toward the distal position after the firing force drops to a force F1. As the firing driver advances toward the distal position, the firing force does not exceed the firing force threshold FTF_{MAX}, so the control system takes no additional actions. However, as discussed above, had the firing force threshold reached or exceeded the firing force threshold FTF_{MAX}, the control system would only make adjustments to the firing algorithm.

Accordingly, the foregoing provides predefined zones that signal to the control system which system (the closure system or the firing system) the control system should leverage should the firing force reach or exceed the firing force threshold. In some embodiments, when the firing driver is in the high co-operative zone CZ_{H}, the control system exclusively leverages the closure system. In some embodiments, when the firing driver is in the low co-operative zone CZ_{L}, the control system exclusively leverages the firing system. In some embodiments, when the firing driver is in the high co-operative zone CZ_{H}, the control system leverages a combination of the firing system and the closure system, as described elsewhere herein. In some embodiments, a middle co-operative zone is defined intermediate the high co-operative zone CZ_{H} and the low co-operative zone CZ_{L}. In some such embodiments, the control system leverages the closure system in the high co-operative zone CZ_{H}, the firing system in the low co-operative zone CZ_{L}, and a combination of both the closure system and the firing system in the middle co-operative zone.

Referring now to FIG. 233A, a method 10430 for controlling a surgical stapling system is provided, according to at least one aspect of the present disclosure. The method 10430 comprises setting 10432 a firing force threshold. In various embodiments, a control system, such as controller 10033 or controller 10051, sets a firing force threshold that is a threshold that would result in a firing motor, such as firing motor 10056, stalling. In various embodiments, the control system sets a firing force threshold that is less than a maximum firing force threshold that would result in the firing motor stalling. Various embodiments regarding how the control system sets a firing force threshold are described elsewhere herein.

The method 10430 further comprises setting 10434 a first co-operative zone over a first portion of a firing stroke of a firing driver and a second co-operative zone over a second portion of the firing stroke. In various embodiments, the first co-operative zone comprises a high co-operative zone CZ_{H} and the second co-operative zone comprises a low co-operative zone CZ_{L}. Various embodiments regarding how the control system sets the co-operative zones and the length of the firing zones are described elsewhere herein.

The method 10430 further comprises advancing 10436 a closure driver to a first position intermediate a proximal position and a distal position. In some embodiments, the control system actuates a closure motor, such as closure motor 10058, to advance a closure driver, such as closure tube 10013, distally.

The method 10430 further comprises advancing 10438 a firing driver toward a fired position. In various embodiments, the control system actuates a firing motor, such as firing motor 10056, which applies a firing force to a firing driver, such as firing driver 10024, to advance the firing driver distally.

The method 10430 further comprises monitoring 10440 the firing force as the firing driver moves toward the fired position. In various embodiments, the surgical stapling system includes a force sensor, such sensor 10039 or sensor 10054, that senses the firing force that the firing motor applies to the firing driver.

The method 10430 further comprises comparing 10442 the firing force to the firing force threshold. In various embodiments, control system is in operation communication with the force sensor and receives the monitored firing force from the force sensor. The control system compares the firing force to the firing force threshold to determine if the firing force is below the firing force threshold or if the firing force has reached or exceeded the firing force threshold.

The method 10430 further comprises determining 10444 which of the first co-operative zone or the second co-operative zone that the firing driver is positioned based on the comparison. In various embodiments, the surgical stapling system includes a position sensor that is in operable communication with the control system and that senses a position of the firing driver as the firing driver moves through the firing stroke. Based on the firing force reaching or exceeding the firing force threshold, the control system interrogates the position sensor to determine which of the first co-operative zone or the second co-operative zone that the firing driver is position.

The method 10430 further comprises controlling 10446 movement of at least one of the closure driver or the firing driver based on the determination. In various embodiments, based on the control system determining that the firing driver is in the first co-operative zone, the control system controls both the closure driver and the firing driver. In some embodiments, controlling movement of the firing driver and the closure driver comprises pausing advancement of firing driver for an amount of time, retracting the firing driver toward a proximal position based on the amount of time elapsing, distally advancing the closure driver from the first position to a second position, and resuming advancement of the firing driver toward the distal position based on the closure driver reaching the second position.

In various embodiments, based on the control system determining that the firing driver is in the second co-operative zone, the control system controls the firing driver, but does not control movement of the closure driver. In some embodiments, controlling movement of the firing driver comprises pausing advancement of firing driver for an amount of time and resuming advancement of the firing driver toward the fired position based on the amount of time elapsing.

As described elsewhere herein, the control system can leverage the closure system and the firing system, alone or in combination with each other, in order to reduce the force to fire the firing driver. In one aspect, the control system can make adjustments to the closure algorithm based on the amount of force that the cam surfaces of the firing driver experience during the firing stroke.

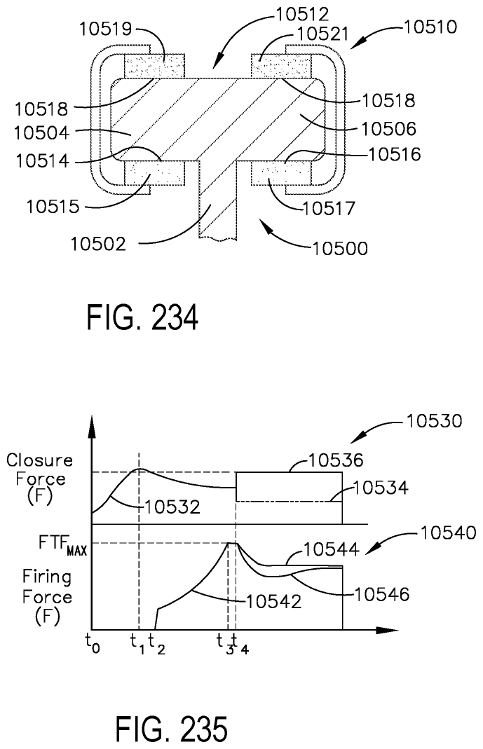

Referring now to FIG. 234, a firing driver 10500 and an anvil 10510 are provided, according to at least one aspect of the present disclosure. In some embodiments, the firing driver 10500 is similar to firing driver 10024. In some embodiments, the anvil 10510 is similar to anvil 10006. The firing driver 10500 comprises a body 10502, a first flange 10504 extending from a first lateral side of the body 10502, and a second flange 10506 extending from a second lateral side of the body 10502. The body 10502 is sized to be received through a longitudinal slot defined in a staple cartridge, such as longitudinal slot 10020 of staple cartridge 10008, during a firing stroke of the firing driver 10500. In some embodiments, the body 10502 comprises a knife, similar to knife 10027, that is configured to cut tissue during the firing stroke of the firing driver 10500.

The anvil 10510 defines a channel 10512 that include a first lower wall 10514, a second lower wall 10516, a first upper wall 10518, and a second upper wall 10520. In use, the first flange 10504 and the second flange 10506 traverse through the channel 10512 during the firing stroke and engage the first lower wall 10514 and the second lower wall 10516, respectively, to maintain a spacing between the anvil 10510 and the staple cartridge during the firing stroke. In some instances, the first flange 10504 and the second flange 10506 engage the first upper wall 10518 and the second upper wall 10520, respectively, when tissue is over-compressed by a closure driver, such as closure tube 10013.

The anvil 10510 further comprises a first force sensor 10515 positioned at the first lower wall 10514, a second force sensor 10517 positioned at the second lower wall 10516, a third force sensor 10519 positioned at the first upper wall 10518, and a fourth force sensor 10521 positioned at the second upper wall 10520. The force sensors 10515, 10517, 10519, 10521 measure forces experienced at their respect walls 10514, 10516, 10518, 10520 by the flanges 10504, 10506 during the firing stroke of the firing driver 10500.

In operation, a control system, such as controller 10033 or controller 10051, receives force measurements from each of the force sensors 10515, 10517, 10519, 10521 in order to determine a clamping state of the anvil 10510. In some embodiments, the clamping state comprises an underclamped state where the first flange 10504 and the second flange 10506 ride along the lower walls 10514, 10516 of the channel 10512, which increases the firing force on the firing driver 10500. In some embodiments, the clamping state comprises an overclamped where the first flange 10504 and the second flange 10506 ride along the upper walls 10518, 10520 of the channel 10512, which increases the firing force on the firing driver 10500. Based on the determination of an underclamping state or an overclamping state, the closure system adjusts the closure algorithm accordingly, as discussed in more detail below.

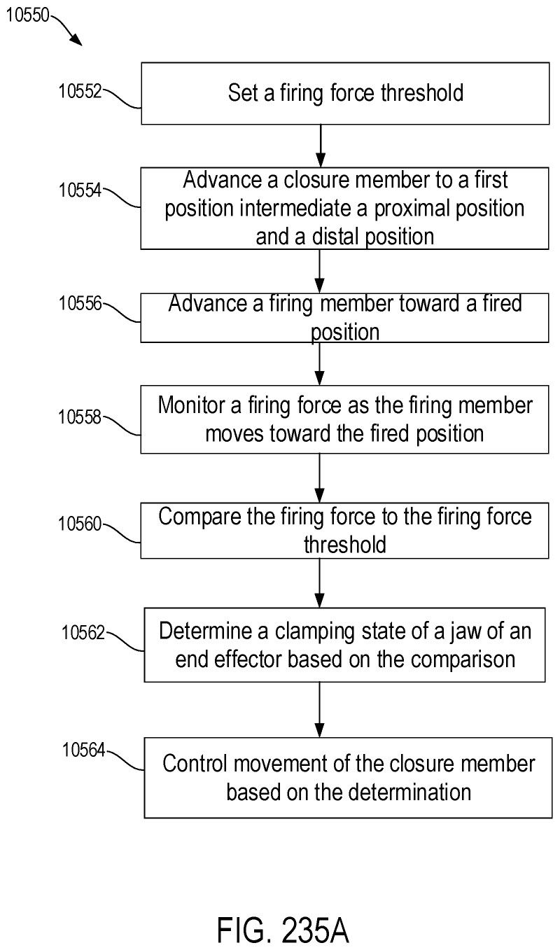

Referring now to FIG. 235, graphs 10530, 10540 are provided that illustrate an exemplary cutting and stapling operation with the firing driver 10500. Graph 10530 illustrates closure force applied by a closure driver, such as closure tube 10013, to a jaw of an end effector, such as anvil 10510, over time. Graph 10540 illustrates the firing force to drive the firing driver 10500 through a firing stroke over time.

At to, the closure driver is advanced distally to apply a closure force 10532 to the anvil 10510 to transition the anvil 10510 toward a closed position. In some embodiments, the closure driver is at a first position intermediate the proximal position and the distal position when the anvil 10510 has reached the closed position. In some embodiments, the closure driver halts advancement toward the distal position once a threshold amount of force has been applied to the tissue by the second jaw, thus preventing the tissue from being damaged. In these embodiments, the second jaw may be in a position intermediate the open position and the closed position (i.e., a partially closed or partially open position) when the closure driver halts advancement.

Once the closure driver stops advancing at t₁, an amount of time is allowed to elapse (t₁ to t₂) before the firing system is initiated. In some embodiments, the amount of time is a predefined amount of time stored in a memory and retrievable by the control system. In some embodiments, the amount of time is variable and a function of the length of the staple cartridge positioned in the end effector. In some embodiments, the amount of time is a function of the closure force applied to the tissue by the anvil 10510. For instance, in some embodiments, the amount of time corresponds to the amount of time take for the closure force applied by the second jaw to drop a predefined amount, indicative of tissue thinning. In some embodiments, the amount of time is a function of how long it took the closure driver to place the anvil 10510 in the closed position. Other amounts of precompression time are described in more detail in U.S. patent application Ser. No. 17/957,946, which is hereby incorporated by reference in its entirety herein.

Once the amount of time has elapsed, at t₂, the control system actuates the firing system to advance the firing driver 10500 through its firing stroke. During the firing stroke, the first flange 10504 and the second flange 10506 traverse through the channel 10512 in the anvil 10510.

As the firing driver 10500 traverses through the firing stroke, the control system monitors the firing force 10542 of the firing driver 10500. In some embodiments, the firing force is measured using a force sensor on the firing driver 10500. In some embodiments, the firing force is measured using a current sensor that measures current through the firing motor that drives the firing driver 10500. Various other sensors for measuring the firing load on the firing driver and firing system are described elsewhere herein.

At t₃, the firing force 10542 reaches a firing force threshold FTF_{MAX}. In some embodiments, the firing force threshold FTF_{MAX} is defined as the threshold that would result in a firing motor that drives the firing driver, such as firing motor 10056, stalling. In some embodiments, the firing force threshold FTF_{MAX} is stored in a memory and retrievable by the control system. In various embodiments, the firing force threshold FTF_{MAX} is based on the type of staple cartridge positioned in the end effector. In various other embodiments, the firing force threshold FTF_{MAX} is set by a user. Based on the firing force 10542 reaches a firing force threshold FTF_{MAX}, the control system determines a clamping state of the anvil 10510.

In various embodiments, the control system determines a clamping state of the anvil 10510 by evaluating the forces sensed by the upper force sensors 10519, 10521 and the lower force sensors 10515, 10517. In various embodiments, the control system compares the forces sensed by the upper force sensors 10519, 10521 to the forces sensed by the lower force sensors 10515, 10517. In some embodiments, the control system determines that the anvil 10510 is in an underclamped state based on the lower force sensors 10515, 10517 sensing forces that are a threshold amount greater than the to the forces sensed by the upper force sensors 10519, 10521. In some embodiments, the control system determines that the anvil 10510 is in an overclamped state based on the upper force sensors 10519, 10521 sensing forces that are a threshold amount greater than the to the forces sensed by the lower force sensors 10515, 10517. In various embodiments, the threshold values for the comparisons can be stored in a memory and retrievable by the control system. In various embodiments, the threshold values are user defined. In some embodiments, the control system determines that the anvil 10510 is in an underclamped state based on the lower force sensors 10515, 10517 sensing forces that exceed a threshold value. In some embodiments, the control system determines that the anvil 10510 is in an overclamped state based on the upper force sensors 10519, 10521 sensing forces that exceed a threshold value.

Based on the determined clamping state of the anvil 10510, the control system adjusts the closure algorithm to change a position of the closure driver. In one embodiment, upon the determination of an overclamped state, the closure driver is retracted proximally, which decreases the closure force 10532 to a first adjusted closure force 10534. Based on the adjustment, the firing force 10542 decreases to a first adjusted firing force 10544. In one embodiment, upon the determination of an underclamped state, the closure driver is advanced distally, which increases the closure force 10532 to a second adjusted closure force 10536. Based on the adjustment, the firing force 10542 decreases to a second adjusted firing force 10546.

Referring now to FIG. 235A, a method 10550 for controlling a surgical stapling system is provided, according to at least one aspect of the present disclosure. With reference now to FIG. 235A together with FIGS. 224 and 225, according to one embodiment of the method 10550a control system, such as controller 10033 or controller 10051, sets 10552 a firing force threshold that is a threshold that would result in a firing motor, such as firing motor 10056, stalling. In various embodiments, the control system sets a firing force threshold that is less than a maximum firing force threshold that would result in the firing motor stalling. Various embodiments regarding how the control system sets a firing force threshold are described elsewhere herein.

According to one embodiment of the method 10550, the control system advances 10552 a closure driver to a first position intermediate a proximal position and a distal position. In some embodiments, the control system actuates a closure motor, such as closure motor 10058, to advance a closure driver, such as closure tube 10013, distally.

According to one embodiment of the method 10550, the control system advances 10554 a firing driver toward a fired position. In various embodiments, the control system actuates a firing motor, such as firing motor 10056, which applies a firing force to a firing driver, such as firing driver 10024, to advance the firing driver distally.

According to one embodiment of the method 10550, the control system monitors 10558 the firing force as the firing driver moves toward the fired position. In various embodiments, the surgical stapling system includes a force sensor, such sensor 10039 or sensor 10054, that senses the firing force that the firing motor applies to the firing driver.

According to one embodiment of the method 10550, the control system compares 10560 the firing force to the firing force threshold. In various embodiments, control system is in operation communication with the force sensor and receives the monitored firing force from the force sensor. The control system compares the firing force to the firing force threshold to determine if the firing force is below the firing force threshold or if the firing force has reached or exceeded the firing force threshold.

According to one embodiment of the method 10550, the control system determines 10562 a clamping state of a jaw of an end effector based on the comparison. In various embodiments, as described elsewhere herein, the control system determines a clamping state of a jaw, such as anvil 10510, by evaluating the forces sensed by upper force sensors 10519, 10521 and lower force sensors 10515, 10517 in a channel, such as channel 10512, of the jaw.

According to one embodiment of method 10550, the control system controls 10564 movement of the closure driver based on the determination. In various embodiments, based on the control system determining the clamping state of the jaw, the control system adjusts the closure algorithm to change a position of the closure driver. In some embodiments, upon the determination of an underclamped state, the closure driver is distally advanced. In some embodiments, upon the determination of an overclamped state, the closure driver is proximally retracted.

During parameterization of a surgical cutting and stapling device, the unloaded closure load and the unloaded firing load can be determined. In one embodiment, the unloaded closure load is the amount of force required to transition a jaw of an end effector, such as the second jaw 10006, from an open positon to a closed position. In one embodiment, the unloaded firing load is the amount of force required to move a firing driver, such as firing driver 10024, through a firing stroke. These values constitute a baseline for the surgical system to use. In some embodiments, these baseline values are stored in a memory, such as memory 10035 or memory 10053.

Additional functions could be included during the parameterization process. In one aspect, a “fake” staple cartridge is inserted into an end effector, such as end effector 10002, and the end effector is actuated to grasp onto simulated tissue materials. One material of the simulated tissue materials is used to simulate an overstressed firing/closure condition and a second material of the simulated tissue materials is used to simulate an understressed firing/closure condition. The results of these three baseline simulations (unloaded, overstressed, and understressed) are used to influence final parameters of closure force thresholds, firing force thresholds and device specific frictional losses.

Furthermore, during parameterization, the surgical system may indicate that the maximum closure force is a threshold amount higher than nominal. As such, the surgical system is adjusted by shifting the maximum closure threshold according. Due to this closure threshold adjustment, the surgical system also adjusts the firing force thresholds to cooperate with the increased closure force. In some embodiments, the adjustment to firing force threshold is a proportional increase to the adjustment in the closure force threshold. In some embodiments, the adjustment to firing force threshold is a 1-to-1 adjustment to the adjustment in the closure force threshold. In some embodiments, based on the shifting maximum closure force threshold, the control system also adjusts the time in which a stall warning signal is provided. In some embodiments, based on the shifting maximum closure force threshold, the control system also adjusts parameters associated with the motor control algorithms. In some embodiments, the parameters include a maximum pulse width modulation signal to the motor controller, such as motor driver 10067, the acceleration of the firing driver by the firing system, or the pausing/pausing sequence in the firing algorithm, or combinations thereof. Other parameters associate with the motor control algorithms are discussed elsewhere herein.

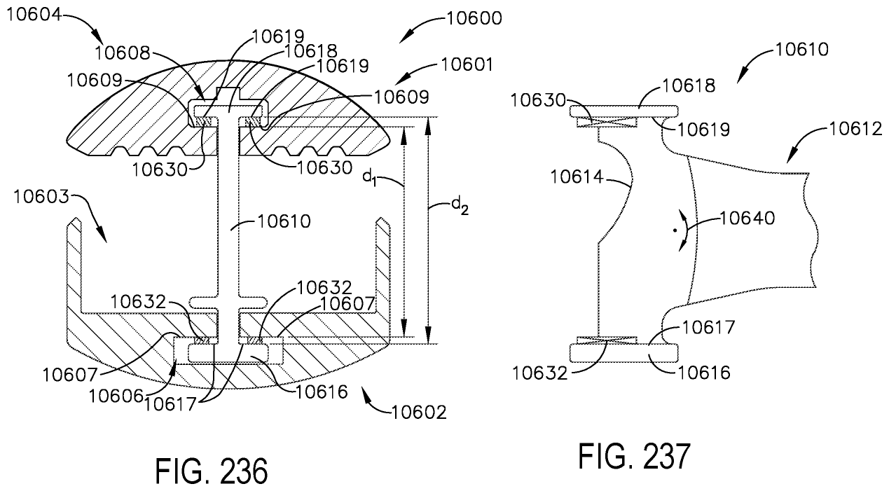

Referring to FIGS. 236 and 237, a surgical stapling system 10600 including an end effector 10601 is provided, according to at least one aspect of the present disclosure. In some embodiments, the end effector 10601 is similar to end effector 10002. The end effector 10601 comprises a first jaw 10602 and a second jaw 10604. The first jaw 10602 comprises an elongate channel 10603 that is sized to receive a staple cartridge, such as staple cartridge 10008, therein. The second jaw 10604 comprises an anvil configured to deform staples ejected from the staple cartridge. The second jaw 10604 is pivotable relative to the first jaw 10602 between an open position and a closed position. The first jaw 10602 defines a first jaw channel 10606 that comprises first surfaces 10607 and the second jaw 10604 defines a second jaw channel 10608 that comprises second surfaces 10609. A first distance d₁ is defined between the first surfaces 10607 and the second surfaces 10609.

The surgical stapling system 10600 further comprises a firing driver 10610 and a firing bar 10612. In some embodiments, the firing driver 10610 is similar to firing driver 10024 and the firing bar 10612 is similar to firing bar 10028. In use, the firing bar 10612 is driven by a firing system, such as firing motor 10056 and firing motor drive assembly 10057, to drive the firing driver 10610 through a firing stroke to deploy staples removably stored in the staple cartridge and to cut tissue captured by the end effector 10601 with a knife 10614, as discussed elsewhere herein. In various embodiments, the firing bar 10612 comprises a plurality of laminated strips.

The firing driver 10610 further includes a first cam 10616 and a second cam 10618. During the firing stroke, the first cam 10616 traverses the first jaw channel 10606 and the second cam traverses the second jaw channel 10608. The first cam 10616 and the second cam 10618 co-operatively maintain the end effector 10601 in the closed state by applying closure forces to the first surfaces 10607 and the second surfaces 10609, respectively. A second distance d₂ is defined between top surfaces 10617 of the first cam 10616 and bottom surfaces 10619 of the second cam 10618.

During the firing stroke, the firing driver 10610 may experience frictional resistance 10630 from the bottom surfaces 10619 of the second cam 10618 engaging and riding along the second surfaces 10609 of the second jaw channel 10608. Similarly, the firing driver 10610 may experience frictional resistance 10632 from the top surfaces 10617 of the first cam 10616 engaging and riding along the first surfaces 10617 of the first jaw channel 10606. As a result of these frictional forces 10630, 10632, the firing driver 10610 may rotate 10640, which will lead to an increase in the amount of force to fire the firing driver 10610.

During the parameterization process, the control system is calibrated with information regarding the end effector 10601 so as to know when the closure system and/or the firing system should be adjusted to regulate the firing force on the firing driver. In some embodiments, the control system, such as controller 10033 or controller 10051, is provided with distance d₁ and distance d₂. As shown in FIG. 236, d₂ is greater than d₁. Accordingly, the control system determines that there is a clearance between the surfaces 10617, 10619 and the surfaces 10607, 10609, which may lead to rotation of the firing driver 10610 during the firing stroke. This assembly level measurement of the end effector 10601 is integrated into the control system such that the control system has a better understanding of when the closure system algorithm and/or the firing system algorithm should be adjusted during the firing stroke of the firing driver. In one aspect, these assembly level measurements are utilized to adjust firing zones of the firing driver. In some embodiments, these assembly level measurements are utilized to adjust the variable firing zones that are established based on the co-operation between the closure system and the firing system, as discussed elsewhere herein. In some embodiments, these assembly level measurements are utilized to adjust the predefined firing zones, such as the high co-operation zone CZ_{H} and the low co-operation zone CZ_{L}, as discussed elsewhere herein. Any number of assembly level measurements can be incorporated into the control system to make adjustments to the co-operative zones of the firing driver. In some embodiments, the assembly level measurements comprise the height of the knife, the height of the firing driver, the height of the first jaw channel, or the height of the second jaw channel, or combinations thereof.

As described elsewhere herein, the control system can leverage the closure system and the firing system, alone or in combination with each other, in order to reduce the force to fire the firing driver. In one aspect, the control system can leverage the energy relief that the closure system provides in combination with pauses and pulsing in the firing algorithm to further reduce the force to fire the firing driver.

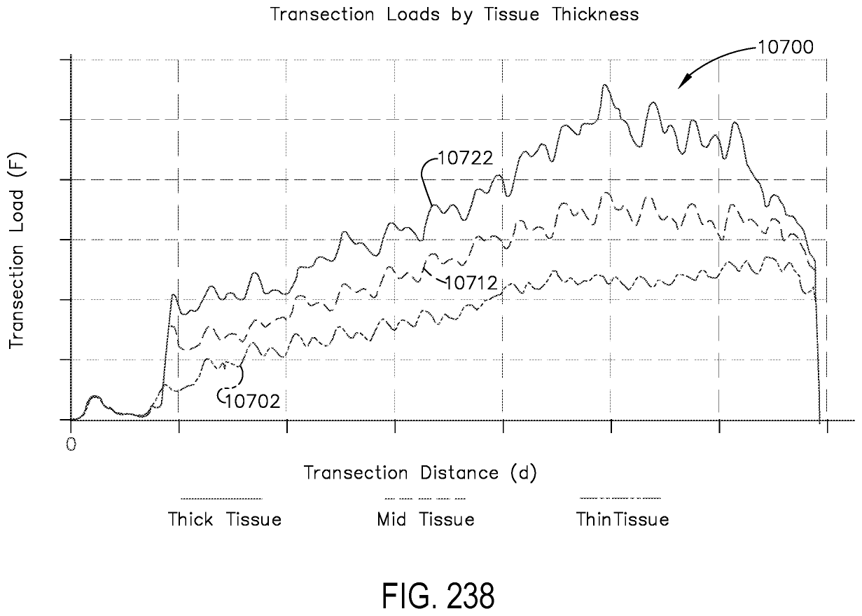

Referring now to FIG. 238, a graph 10700 is provided that illustrates the relationship between firing force over time for various firing strokes of firing drivers for varying tissue thicknesses. As shown in graph 10700, during a first firing stroke with a firing driver, such as firing driver 10024, through thin tissue, the firing force has a first firing force profile 10702. In the first firing force profile 10702, the force to fire the firing driver reaches a first firing force peak 10704. After the first firing force peak 10704, the force to fire the firing driver increases at a first rate.

During a second firing stroke with the firing driver, the firing force has a second firing force profile 10712 where the forces experienced are greater than in the first firing force profile 10702. In the second firing force profile 10712, the force to fire the firing driver reaches a second firing force peak 10714 that is greater than the first firing force peak 10704. After the second firing force peak 10714, the force to fire the firing driver increases at a second rate.

During a third firing stroke with the firing driver, the firing force has a third firing force profile 10722 where the forces experienced are greater than in the first firing force profile 10702 and the second firing force profile 10712. In the third firing force profile 10722, the force to fire the firing driver reaches a third firing force peak 10724 that is greater than the first firing force peak 10704 and the second firing force peak 10714. After the third firing force peak 10724, the force to fire the firing driver increases at a third rate.

In various embodiments, the control system can predict the firing force profiles, such as the firing force profiles 10702, 10712, 10722, based on the initial firing force readings, such as the initial firing force peaks 10704, 10714, 10724, the rate at which the firing force changes from the initial firing force peaks, or the number of hills and valleys detected in the firing force profile after an amount of time has elapsed from the initial firing force peaks 10704, 10714, 10724, or a combination thereof. Based on the prediction, the control system adjusts the closure algorithm and/or firing algorithm, as discussed elsewhere herein, to reduce the firing force profile later in the transection cycle.

In one embodiment, a staple cartridge is inserted into a first jaw, such as first jaw 10004, of an end effector, such as end effector 10002. The end effector is then utilized to grasp tissue within the jaws of the end effector. In particular, a closure driver, such as closure tube 10013, is advanced distally to apply a closure force to a second jaw, such as second jaw 10006, to transition the second jaw toward the closed position. A firing system, such as firing motor 10056 and firing motor drive assembly 10057, then advances a firing driver, such as firing driver 10024, through its firing stroke to deploy staples from the staple cartridge and cut the tissue captured by the end effector.

As the firing driver is advanced through its firing stroke, the control system monitors the firing force of the firing driver. In some embodiments, the firing force is measured using a force sensor on the firing driver. In some embodiments, the firing force is measured using a current sensor that measures current through the firing motor that drives the firing driver. Various other sensors for measuring the firing load on the firing driver and firing system are described elsewhere herein.

At a point in the firing stroke of the firing driver, the control system detects an initial firing force peak. In one aspect, the initial firing force peak is indicative of the firing driver beginning to transect the tissue captured by the end effector. In one aspect, the initial firing force peak is indicative of the firing driver beginning to deploy staples from the staple cartridge.

Based on the detection, the control system compares the initial firing force peak value to threshold values stored in a memory, such as a memory 10053. In various embodiments, the threshold values correspond to the force values that are expected of different tissue thicknesses. In one embodiment, should the initial firing force peak value exceed a first threshold value but remain below a second threshold force value greater than the first threshold value, the control system determines that thin tissue is positioned within the jaws of the end effector. In another embodiment, should the initial firing force peak value exceed the second threshold value but remain below a third threshold force value greater than the second threshold value, the control system determines that medium tissue is positioned within the jaws of the end effector. In another embodiment, should the initial firing force peak value exceed the third threshold value, the control system determines that thick tissue is positioned within the jaws of the end effector. Based on the comparison, the control system predicts future firing forces that are expected to be experienced by the firing driver during the firing stroke.

In various other embodiments, after the detection of the initial firing force peak, the control system detects the rate at which the firing force increases from the initial firing force peak. Based on the initial firing force peak and the rate at which the firing force increases from the initial firing force peak, the control system predicts future forces to fire in the firing stroke. In various other embodiments, the control system detects the number of peak and valleys in the firing force over a portion of the firing stroke after the initial firing force peak. Based on the initial firing force peak and the number of peaks and valleys detected, the control system predicts future forces to fire in the firing stroke.

In some embodiments, based on the prediction, the control system adjusts the closure algorithm to displace the closure driver. In one embodiment, upon the determination of thin tissue being positioned in the jaws of the end effector, the control system advances the closure driver a first amount. In one embodiment, upon the determination of medium tissue being positioned in the jaws of the end effector, the control system advances the closure driver a second amount different than the first amount. In various embodiments, the second amount is greater than the first amount. In one embodiment, upon the determination of thick tissue being positioned in the jaws of the end effector, the control system advances the closure driver a third amount which is different than the second amount. In various embodiments, the third amount is greater than the second amount.

In some embodiments, based on the prediction, the control system sets zones for the firing stroke, where the zones comprise lengths of the firing stroke where the closure algorithm is exclusively adjusted, the firing algorithm is exclusively adjusted, or a combination of both the closure algorithm and the firing algorithm are adjusted, as discussed elsewhere herein.

In some embodiments, based on the prediction, the control system adjusts predefined zones, such as the high co-operative zone CZ_{H} and the low co-operative zone CZ_{L} for the firing stroke. In some embodiments, based on the prediction, the control system adjusts the closure algorithm and/or the firing algorithm for different portions of the firing stroke. In some embodiments, based on the prediction, the control system adjusts the length and the number of occurrences of pauses of the firing driver during the firing stroke.

Accordingly, based on a prediction from an early portion of the, the control system is able to make adjustments that will result in lower forces to fire later in the firing stroke.

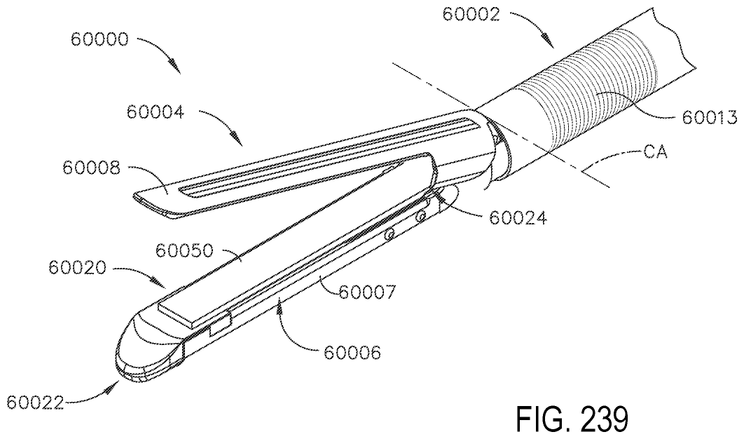

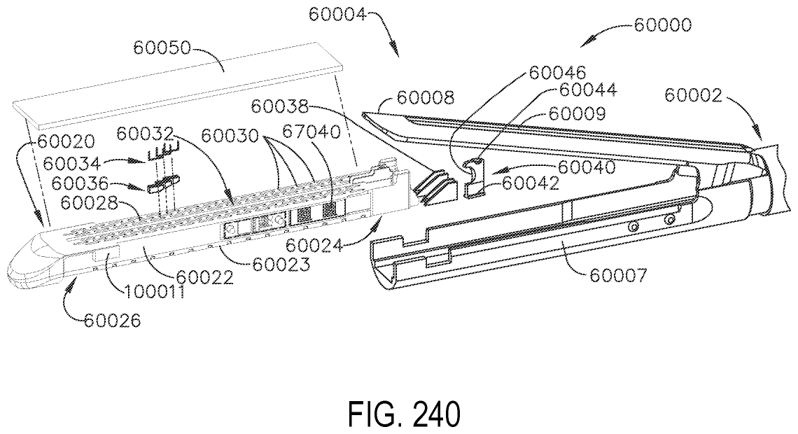

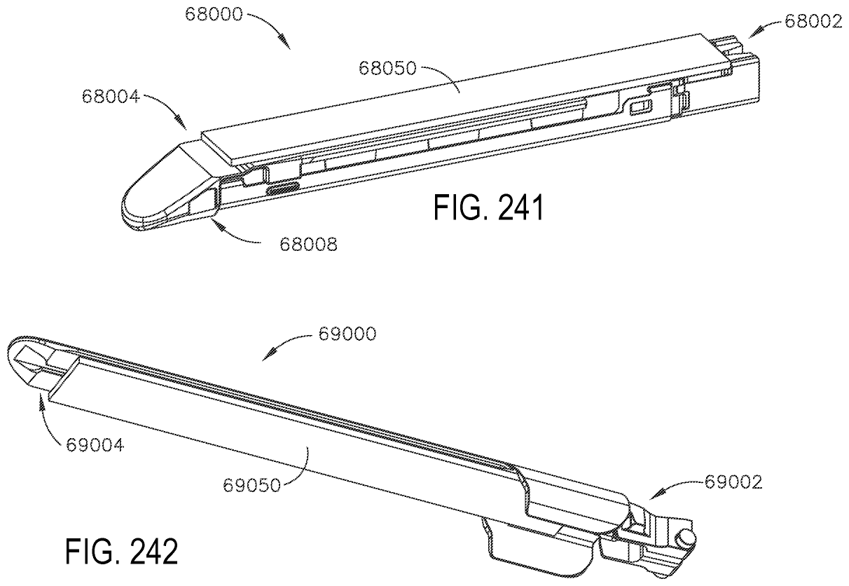

Referring to FIG. 239, a surgical stapling system 60000 is shown. The surgical stapling system 60000 comprises a shaft 60002 and an end effector 60004 extending from the shaft 60002. The end effector 60004 comprises a first jaw 60006 and a second jaw 60008. The first jaw 60006 comprises an elongate channel 61007 and a staple cartridge 60020. The staple cartridge is configured to be replaced during a surgical procedure. Other embodiments are envisioned in which the staple cartridge 60020 is not removable from, or at least readily replaceable from, the first jaw 60006.

The second jaw 60008 comprises an anvil configured to deform staples ejected from the staple cartridge 60020. The second jaw 60008 is pivotable or otherwise movable relative to the first jaw 60006 about a closure axis CA between an open position and a closed position. The surgical stapling system 60000 further comprises an articulation joint 60013 configured to permit the end effector 60004 to be rotated, or articulated, relative to the shaft 60002. The end effector 60004 is rotatable about an articulation axis extending through the articulation joint. Other embodiments are envisioned which do not include an articulation joint.

The staple cartridge 60020 comprises a cartridge body 60022. The cartridge body includes a proximal end 60024, a distal end 60026, and a deck 60028 extending between the proximal end and the distal end. In use, the staple cartridge 60020 is positioned on a first side of the tissue to be stapled and the anvil 60008 is positioned on a second side of the tissue. The anvil 60008 is moved toward the staple cartridge 60020 to compress and clamp the tissue against the deck 60028. Thereafter, a plurality of staples 60034 that are removably stored in the cartridge body 60022 are deployed into the tissue. The staples 60034 are removably stored in corresponding staple cavities 60030 formed in the cartridge body 60022. The staple cavities 60030 are arranged in six longitudinal rows. Three rows of staple cavities 60030 are positioned on a first side of a longitudinal slot 60032 and three rows of staple cavities 60030 are positioned on a second side of the longitudinal slot 60032. Other arrangements of staple cavities and staples may be possible.

The staples 60034 are supported by staple drivers 60036 supported in the staple cavities 60030. Staples supported on staple drivers can be seen in U.S. Pat. No. 9,844,369, entitled, SURGICAL END EFFECTORS WITH FIRING ELEMENT MONITORING ARRANGEMENTS, the entire disclosure of which is hereby incorporated by reference herein. The drivers 60036 are movable between a first, or unfired position, and a second, or fired, position to eject the staples 60034 from the staple cavities 60030. The drivers 60036 are retained in the cartridge body by a retainer 60023 which extends around the bottom of the cartridge body 60022 and includes resilient members configured to grip the cartridge body and hold the retainer to the cartridge body. The drivers 60036 are movable between their unfired positions and their fired positions by a sled 60038. The sled 60038 is movable between a proximal position adjacent the proximal end 60024 of the cartridge body 60022 and a distal position adjacent the distal end 60026 of the cartridge body 60022. The sled 60038 comprises a plurality of ramped surfaces configured to slide under the drivers 60036 and lift the drivers, and the staples 60034 supported thereon, toward the anvil 60008.

Further to the above, the sled 60038 is moved distally by a firing actuator or firing member 60040. The firing actuator 60040 is configured to contact the sled 60038 and push the sled 60038 toward the distal end 60026. The longitudinal slot 60032 defined in the cartridge body 60022 is configured to receive the firing actuator 60040. The anvil 60008 also includes a slot 60009 configured to receive the firing actuator 60040. The firing actuator 60040 also comprises a knife 60046 configured to incise the tissue captured intermediate the staple cartridge 60020 and the anvil 60008.

In many instances, buttresses, tissue thickness compensators, and/or adjuncts (collectively referred to herein as “adjuncts”) 60050 are used to reinforce a staple line and provide support to the tissue surrounding the staple line. For example, an adjunct 60050 may be supported on the cartridge deck 60028. An adjunct, when used in connection with a replaceable staple cartridge, may be referred to herein as a “replaceable staple assembly”, for example. In use, the end effector is positioned so as to capture the tissue to be stapled between the adjunct material and the underside of the anvil or the cartridge deck. Once the end effector has been properly positioned, the anvil is closed. The stapling instrument is then fired causing the firing actuator to move distally through the end effector. As the firing actuator moves distally, the firing actuator drives the sled or other camming arrangement into contact with the drivers in the staple cartridge that support the staples thereon. As the sled contacts the drivers, the drivers are driven toward the closed anvil driving the staples through the adjunct material and the clamped target tissue into forming contact with the underside of the anvil. The trailing knife on the firing actuator cuts through the stapled adjunct material and tissue until the firing actuator reaches the distal end of the staple cartridge and all of the staples stored therein have been fired. The anvil of the end effector is then opened and the stapled tissue is freed from the end effector. The adjunct material remains with the stapled tissue and eventually gets absorbed by the patient's body.

As discussed above, when a surgical procedure involves a long tissue cutline that requires use of multiple staple cartridges as well as corresponding multiple adjuncts, the subsequent adjunct is overlapped with a previously installed adjunct to form continuous lines of adjunct material on each side of the cut tissue throughout the length of the tissue cut line. When the knife of the firing actuator contacts the overlapped conventional adjuncts, the resistance created by overlapping material may cause the adjuncts to undesirably bunch, move or skew which might result in misalignment of the staples with the forming pockets in the anvil during firing and also may result in previously formed staples being cut through or otherwise damaged by the knife.

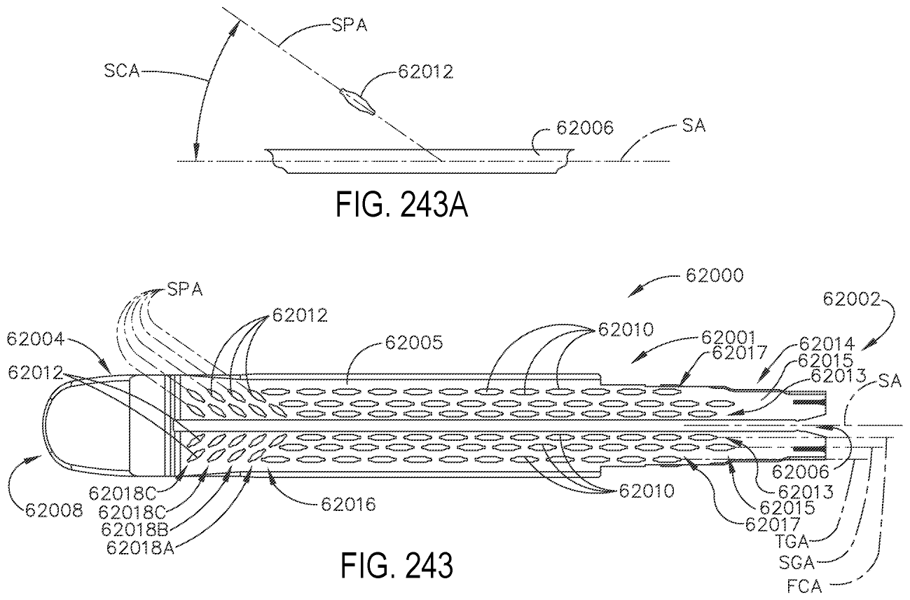

A staple cartridge 62000 configured to address the foregoing problem is illustrated in FIG. 243. The staple cartridge 62000 is configured to be seated in and removable from a first jaw as discussed above; however, other embodiments are envisioned in which a staple cartridge is not removable from, or at least readily replaceable from, a first jaw. The staple cartridge 62000 comprises a cartridge body 62001 and a nose 62008. The cartridge body 62001 comprises a proximal end portion 62002, a distal end portion 62004, and a cartridge deck 62005 extending between the proximal end portion 62002 and the distal end portion 62004. The cartridge body 62001 also comprises a longitudinal slot 62006 which extends through the cartridge body 62001 from the proximal end portion 62002 to the distal end portion 62004. The cartridge body 62001 includes a plurality of first staple cavities 62010 and a plurality of second staple cavities 62012. Each staple cavity 62010, 62012 movably supports a corresponding staple driver therein. A corresponding staple is removably stored on the driver and is configured to be ejected out of the staple cavity 62010, 62012 by the driver during a firing stroke of the firing actuator.

As can be seen in FIG. 243, the first staple cavities 62010 are arranged in six longitudinal rows that extend from a location in the proximal end portion 62002 of the deck surface 62005 and extend distally to a distal end portion 62106 of the deck surface 62005. Three rows of first staple cavities 62010 are positioned on a first side of the longitudinal slot 62006 and three rows of first staple cavities 62010 are positioned on a second side of the longitudinal slot 62006. As can be seen in FIG. 243, each of the first staple cavities 62010 are parallel with or substantially parallel with the slot 62006. The first staple cavities 62010 in a second row 62015 are staggered relative to the first staple cavities 62010 in a first row 62013 of first staple cavities 62010 that is adjacent to the slot 62006. The first staple cavities 62010 in a third row 62017 are staggered relative to the first staple cavities 62010 in the second row 62015. Such arrangement results in overlapping staple lines.

Still referring to FIG. 243, the second staple cavities 62012 open through the distal portion 62016 of the deck surface 62005. As illustrated in FIG. 243, the second staple cavities 62012 are arranged in a different pattern than that of the first staple cavities 62010. The first staple cavities 62010 are positioned substantially parallel to the longitudinal slot 62006 whereas the second staple cavities 62012 are positioned at an angle with respect to the longitudinal slot 62006. In some instances, the staple cavities 62012 are positioned at an angle less than 90° with respect to the longitudinal slot 62006. For example, as shown in FIG. 243A, the longitudinal slot 62006 may define a slot axis SA and each second cavity 62012 may define a second cavity axis SPA. The second staple cavities 62012 can arranged in a line of two second staple cavities 62012 wherein each line is positioned so that the angle SCA between the slot axis SA and the second cavity axis SPA may be between 90° and 20°. Such angling of the second cavities 62012 relative to the longitudinal slot 62006 results in only two second staple cavities 62012 being provided in each line of second staple cavities. In the illustrated arrangement, four lines 62018A, 62018B, 62018C, and 62018D are employed on each side of the longitudinal sot 62006. Other numbers of lines of second cavities 62012 are contemplated.

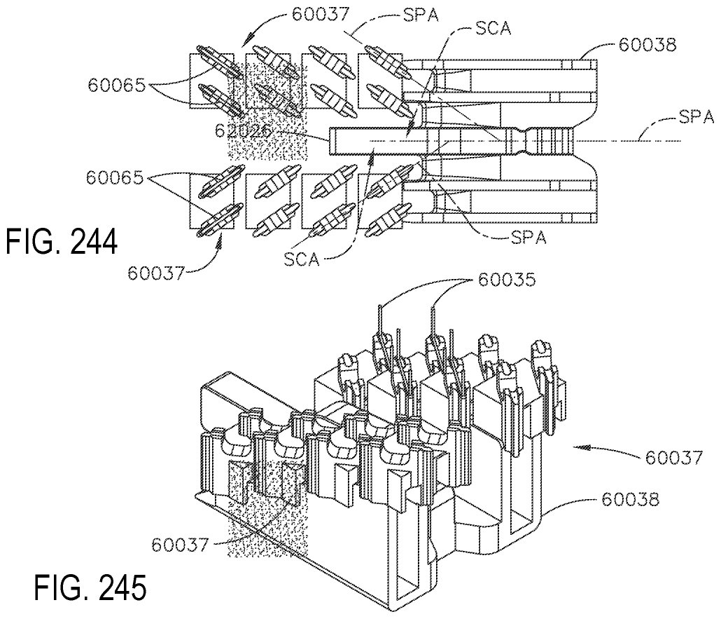

Referring To FIGS. 244 and 245, there is shown a pair of angled staple drivers staple drivers 60037 that serve to support staples 60035 thereon in the second staple cavities 62012. As can be seen in those Figures, the staples 60035 are supported at angles SCA relative to the slot axis SA. The staple drivers 60037 are driven upwardly in their respective second staple cavities 62012 as the sled 60038 is driven distally during the firing stroke.

The staple cartridge 62000 differs from other staple cartridges that may employ entire lines staple cavities and staples that are biased at an angle relative to the longitudinal slot axis SA. The staple cartridge 62000 maintains the advantages provided by the three lines of parallel staples on each side of the longitudinal slot, but also gains an improved advantage provided by the few rows of second staple cavities and staples biased at an angle relative to the longitudinal slot and the axis of travel of the firing actuator. As discussed above, when completing multiple sequential firings, situations occur where the adjunct of a second firing stroke overlaps with the adjunct of a first firing stroke. In such instances, the overlap of adjuncts creates a certain thickness which makes cutting more difficult. This increase in cutting resistance can cause the second adjunct to bunch and skew which can divert the knife through some of the formed staples securing the distal end of the first adjunct material to the tissue. The angled pattern of the staples 60035 provides some relief in these instances by providing diversion paths for the knife during subsequent firings. For example, the angled pattern of the staples 60035 allows the knife to pass through a reinforced staple line more easily during a subsequent firing. In some instances, the knife of subsequent firings will impact angled staples 60035 at a low angle which increases the probability that the knife will bounce off of the angled staples 60035 and pass by during the firing stroke instead of jamming and distorting the shape and formation of the angled staples 60035. As such, the position of the staple cavities 62012 and staples 60035 reduces the likelihood that the force of the knife through a previously fired staple line will impart damaging forces on the staples 60035 within the previously fired staple line. In addition, by providing the staples 60035 at an angle to the direction of travel of the firing actuator, the staples 60035 may provide an increased resistance to movement of the second adjunct material as the knife of the firing actuator initially contacts the second adjunct to cut and drive therethrough.

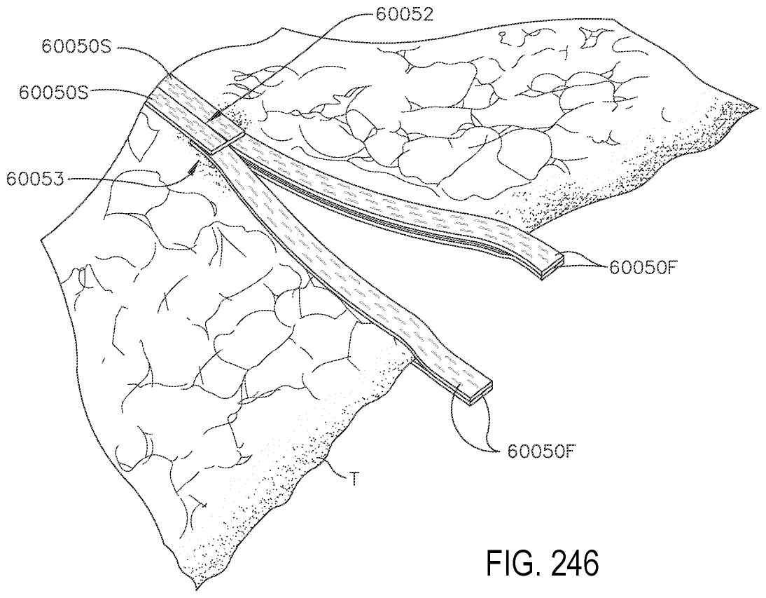

FIG. 246 illustrates the use of staple cartridges 62000 to sequentially cut through tissue “T” when using a pair of first adjuncts 60050F and a pair of second adjuncts 60050S. The first adjuncts 60050F are stapled one both side of the tissue T using a first staple cartridge 62000 in the manners described herein. In such instance, one first adjunct 60050F is positioned on the cartridge deck and the other first adjunct 60050F is placed adjacent the underside of the anvil. The target tissue “T” is then positioned between the first adjuncts 60050F and then the anvil is closed and the firing actuator is fired. Once the first staple cartridge 62000 is fully fired and spent, it is removed from the end effector and replaced with another fresh staple cartridge 62000 as well as the pair of second adjuncts 60050S. The end effector is then positioned so that the proximal end portions 60051 of the second adjuncts 60050S overlap the distal end portions 60053 of the first adjuncts 60050F. The distal end portions 60053 of the first adjuncts 60050F were stapled with staples 60035 that were supported in the angled second cavities 62012 in the first staple cartridge 62000. The anvil of the end effector is then closed to capture the uncut tissue between the second adjuncts 60050S and the firing actuator is fired. As the firing actuator is moved distally, it contacts the four layers of overlapping adjuncts 60050F, 60050S which increases an amount of resistance experienced by the knife of the firing actuator. In the event that the adjuncts become skewed or bunched up, the angled orientation of the staples 60035 will increase the likelihood that the knife will bounce off staples 60035 and pass thereby without cutting through those previously formed staples. Alternative cartridge arrangements are contemplated wherein the second staple cavities 62012 (and staples 60035 supported thereon) are provided only in a proximal portion of the cartridge, and in other arrangements, the second staple cavities 62012 (and staples 60035 supported thereon) are provided only in a distal portion and a proximal portion. However, those angled cavities do not extend the entire length of the cartridge. A central segment of cavities comprising three lines of staple cavities that are parallel to each other and the slot axis separate the proximal segment of angled cavities from the distal segment of angled cavities on both sides of the longitudinal slot in such embodiment.

The various adjuncts disclosed herein may be used in conjunction with the stapling system described above as well as other known stapler arrangements and systems to reinforce a staple line and provide support to the tissue surrounding the staple line. The adjunct arrangements discussed below may also address the problems outlined above by employing a change in the cross-section, pattern or integrity of the adjunct material to predefine locations wherein the adjunct material is configured to advantageously tear or separate. In such instances, the adjunct comprises a predefined and specific weakened portion or portions of the adjunct material to prevent a first adjunct from impacting the performance of a second adjunct during multiple sequential staple firings. Such weakened portion(s) comprise intermittent openings, and/or interruptions, and/or thinned out, tearable portions configured to tear during multiple sequential staple firings. The tearable portions are configured to enable the adjunct to shear in predefined ways. The tearable portions are also configured to enable the separation of specific staples from the adjunct upon the occurrence of dragging forces in some instances. By reducing the strength of certain portions of the adjunct, staple retaining or holding forces are reduced during a second cutting motion of the knife during multiple sequential staple firings.

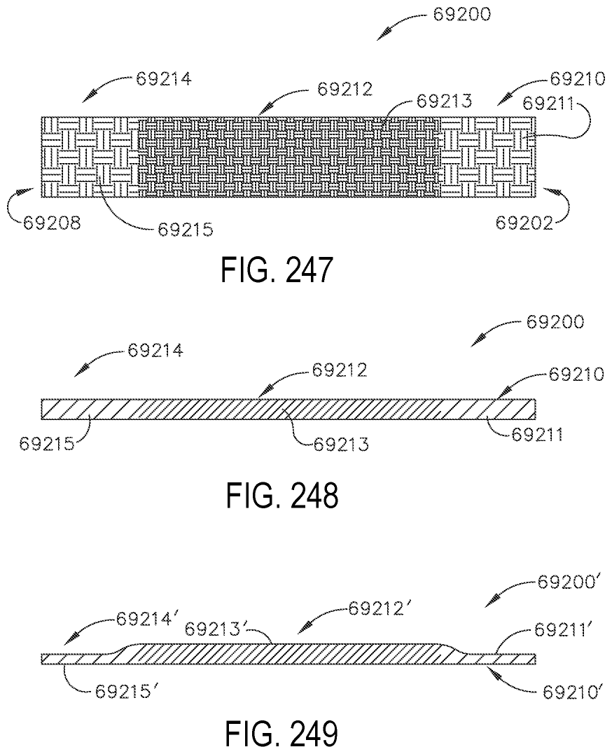

Turning to FIGS. 247 and 248, one adjunct 69200 of the present invention comprises a first region 69210, a second region 69212, and a third region 69214. The first region 69210 of the adjunct 69200 is located at the proximal end 69202 of the adjunct 69200. The third region 69214 is located at the distal end 69208 of the adjunct 69200. The second region 69212 is located in between the first region 69210 and the third region 69214. As seen in FIG. 247, the first region 69210 comprises a woven mesh 69211. The second region 69212 comprises a woven mesh 69213, and the third region 69214 comprises a woven mesh 69215. While the woven meshes 69211, 69213, 69215 may be formed from the same material (or different materials otherwise laminated or knitted together), the meshes 69211 and 69215 have attributes and strength characteristics that differ from the attributes of the woven mesh 69213. For example, all of the woven meshes 69211, 69213, and 69215 may comprise Vicryl® material or other woven materials disclosed herein. However, the material comprising the woven meshes 69211 and 69215 may have a different knot density, strand diameter, weave and/or denier, that differs from the knot density, strand diameter, weave and/or denier of the woven mesh 69213. The denier and/or number of strands forming the woven mesh 69213 may be higher than the denier and/or the number of stands forming the woven meshes 69211 and 69215. For example, the woven mesh 69213 may have 2×28 denier strands and the woven meshes 69211 and 69215 may have 1×28 denier strands, yet have the same weave pattern. In some instances, the weave of the mesh materials 69211 and 69215 is looser than the weave of the mesh material 69213. As can be seen in FIG. 248, while the compositions of the first region 69210 and the third region 69214 are the same, they differ from the composition of the second region 69212. However, in the illustrated example, the cross-sectional thicknesses of the regions 69210, 69212, and 69214 are the same. In such embodiments, the first and third regions 69210 and 69214 may be further weakened by exposing those portions (but not the second portion 69212) to one or more of moisture, ultraviolet light, and radiation (gamma, X-ray, E-beam, etc.) during manufacturing and prior to use.

In the above-described arrangements, the woven mesh 69213 comprises a greater strength than the woven meshes 69211 and 69215. This means that the woven mesh 69211 and woven mesh 69215 are easier to cut through and offer less resistance to the knife than the woven mesh 69213. In addition, the woven meshes 69211 and 69215 may be more likely to break free of the staples fired therethough should the adjunct material start to bunch or plow during firing. Thus, in applications wherein the first region 69210 of a second adjunct 69200 is overlapped over a third region 69214 of a previously-stapled, first adjunct 69200, the second adjunct 69200 is less likely to plow or bunch up when initially contacted by the knife. Further, in the event that second adjunct 69220 nonetheless starts to bunch or plow, the woven mesh 69215 forming the third region 69214 of the first adjunct 69200 is more likely to break away from the formed staples therein, leaving those staples fastened to the tissue. Likewise in such instance, the woven mesh 69211 forming the first region 69210 in the second adjunct 69200 is more likely to break away from any of the staples initially formed therein leaving those staples fastened to the underlying tissue. Thus, the various forms of adjunct 69200 serve to address the problems discussed above when using conventional adjuncts in sequential stapling and cutting applications.

Another adjunct 69200′ is illustrated in FIG. 249. The adjunct 69200′ comprises a first region 69210′, a second region 69212′, and a third region 69214′. The first region 69210′, the second region 69212′ and the third region 69214′ may be similar to the first region 69210, the second region 69212, and the third region 69214, respectively described above. However, as can be seen in FIG. 249, the first region 69210′ and the third region 69214′ comprise equal thicknesses 69211′ and 69215′, respectively, whereas the second region 69212′ comprises a different, or greater thickness 69213′ than the thicknesses 69211′ and 69215′. Alternative embodiments comprise further weakening the first and third regions 69210′ and 69214′ by exposing those portions (but not the second portion 69212) to one or more of moisture, ultraviolet light, and radiation (gamma, X-ray, E-beam, etc.) during manufacturing and prior to use. The adjunct 69200′ is configured to provide similar benefits to that of the adjunct 69200 disclosed above.

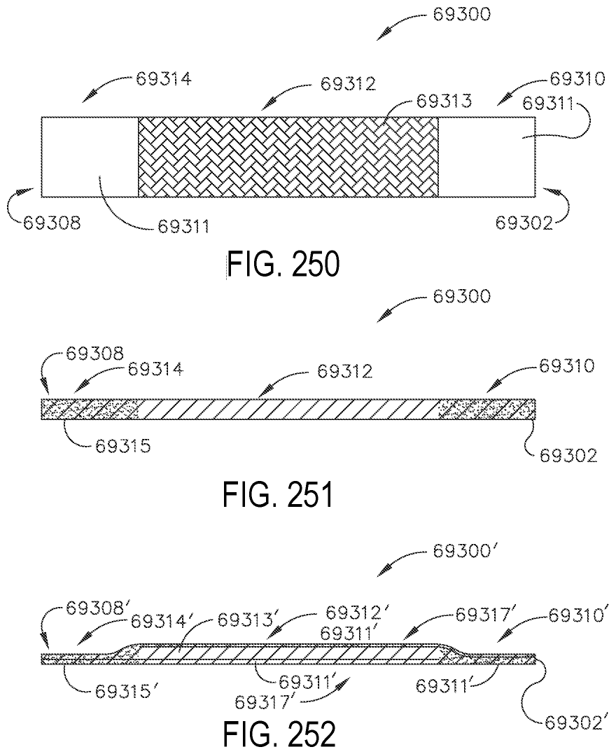

Turning to FIGS. 250 and 251, another adjunct 69300 of the present invention is disclosed. FIG. 251 is a cross-sectional side view of the adjunct 69300. The adjunct 69300 comprises a first region 69310, a second region 69312, and a third region 69314. The first region 69310 of the adjunct 69300 is located at the proximal end 69302 of the adjunct 69300. The third region 69314 is located at the distal end 69308 of the adjunct 69300. The second region 69312 is located between the first region 69310 and the third region 69314. As seen in FIG. 250, the third region 69312 comprises a woven mesh 69313 such as a woven Vicryl® material or any of the other woven materials disclosed herein. The first region 69310 and the second region 69314 comprise a non-woven material 69311 which may comprise any of the non-woven materials described below. The non-woven material 69311 is weaker than the woven material 69313 and provides less reinforcement to the staples formed therein. The material 69311 also provides less resistance to the knife as it is driven therethrough and thereby reduces the likelihood of plowing and bunching occurring during firing. Thus, in use, should the adjunct 69300 start to bunch or plow during cutting, the weaker first region 69310 and the weaker third region 69314 would be more likely to break away from the staples formed therein which may prevent those staples from being pulled out and/or damaged as the firing actuator passes therethrough. Alternative embodiments comprise further weakening the first and third regions 69310 and 69314 by exposing those portions (but not the second region 69312) to one or more of moisture, ultraviolet light, and radiation (gamma, X-ray, E-beam, etc.) during manufacturing and prior to use.

FIG. 252 depicts another adjunct 69300′ that is similar in many respects to the adjunct 69300 described above. As seen in FIG. 252, the adjunct 69300′ comprises a first region 69310′, a second region 69312′, and a third region 69314′. The first region 69310′ and the third region 69314′ comprise cross-sectional thicknesses that differ from the cross-sectional thickness of the second region 69312′. In the illustrated arrangement, the cross-sectional thickness of the second region 69312′ is greater than the cross-sectional thickness of the first region 69310′ and the cross-sectional thickness of the third region 69314′. In one arrangement, the second region 69312′ comprises a woven mesh 69313′ such as a woven Vicryl® or other woven material disclosed herein. The first region 69310′ and the third region 69314′ comprise a non-woven material 69311′ such as PDO, ORC, etc. described below. The non-woven material 69311′ is weaker than the woven material 69313′ and provides less reinforcement to the staples formed therein. In the illustrated arrangement, layers 69317′ of the non-woven material 69311′ are laminated on both sides of the woven material 69313′. In such arrangement, the second region 69312′ comprises both materials 69313′ and 69311′. However, the first region 69310′ and the third region 69314′ are comprised solely of the laminated layers of non-woven material 69311′. The woven material 69313′ does not extend into the first region 69310′ and the third region 69314′. The adjunct 69300′ is configured to provide similar benefits to that of the adjunct 69300 disclosed above. Alternative embodiments comprise further weakening the first and third regions 69310′ and 69314′ by exposing those portions (but not the second region 69312′) to one or more of moisture, ultraviolet light, and radiation (gamma, X-ray, E-beam, etc.) during manufacturing and prior to use.

As discussed above, the differences in material composition can be accomplished by decreasing or increasing the knot density of the material, increasing or decreasing the strand diameter of the material, the denier, or using different materials in conjunction with one another. In other arrangements, the first material may comprise a woven material such as Vicryl® or the like and extend the entire length of the adjunct. Layers of non-woven polymeric material of the types disclosed below may be laminated to the woven material. In one such arrangement, the first material may be exposed to one or more of moisture, ultraviolet light, and radiation (gamma, X-ray, E-beam, etc.) prior to the lamination process. In other arrangements, the entire adjunct is exposed to one or more of moisture, ultraviolet light, and radiation (gamma, X-ray, E-beam, etc.) after the lamination process. In either case, the adjuncts are weakened to enable the knife to pass through the materials without causing bunching or plowing and to enable the adjunct to break away from formed staples if such bunching or plowing inadvertently occurs during firing.

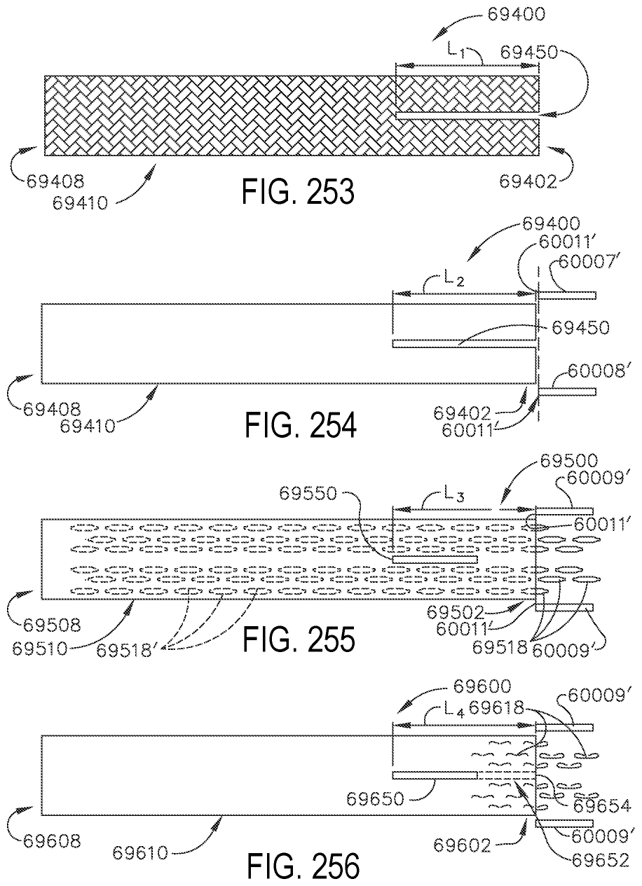

FIG. 253 depicts another adjunct 69400 that comprises a woven material 69410 of the various types and compositions disclosed herein. However, other embodiments are contemplated wherein the adjunct 69400 may be fabricated from any of the other non-woven materials and compositions described herein. The adjunct comprises a proximal end 69402 and a distal end 69408. As can be seen in FIG. 253, a slot 69450 begins at a proximal end 69402 of the adjunct 69400 and extends a distance L1 into the adjunct 69450. As discussed above, when two adjuncts 69400 are used during sequential firing motions, the proximal end 69402 of a second adjunct 69400 will overlay the distal end 69408 of the first adjunct 69400. The slot 69450 provides a unique benefit of allowing for a knife to pass through the slot 69450 in the second adjunct 69400 where the two adjuncts 69400 overlap without encountering two layers of material thereby reducing the amount of resistance encountered by the knife in that region which might otherwise have led to moving, bunching or plowing of the second adjunct 69400. Thus, in at least one embodiment, length L1 of the slot 69450 should be at least as long as the amount that a second adjunct 69400 is overlapped with a previously stapled adjunct to afford the knife unimpeded travel through the second adjunct 69400 in the area of adjunct overlap. In one arrangement, the slot 69450 is wide enough so that a knife of a surgical stapler and firing actuator may pass therethrough without contacting the adjunct material on the sides of the slot. In the illustrated arrangement, slot 69450 extends completely through the thickness of the adjunct material 69410 for the distance L1. Other embodiments are contemplated wherein the slot 69450 only extends partially through the cross-sectional thickness of the adjunct material 69410 for the distance L1. Alternative embodiments are contemplated wherein the slot 69450 comprises a slit. The slit may extend completely through the cross-sectional thickness of the adjunct material 69410 for the distance L1 or it may only extend partially through the cross-sectional thickness of the adjunct material 69410 for the distance L1. In all embodiments, the slot 69450 is intended to eliminate or reduce the amount of resistance encountered by the knife as it passes through the proximal end of the adjunct 69400 (in the area of adjunct overlap).

In various surgical procedures, to ensure that all of the tissue that is clamped between the jaws of the stapling instrument is stapled before being severed by the knife, the anvil of the stapler is commonly formed with “tissue stops” that prevent the tissue from extending proximally in the jaws beyond the proximal-most staples. FIG. 242 illustrates an anvil 60008′ that has a pair of tissue stops 60009′ formed on a proximal end portion thereof. Thus, when the anvil 60008′ is in the open position and the target tissue is positioned between the anvil and the staple cartridge, the tissue contacts the distal edges 60011′ of the tissue stops 60009′ and is thereby prevented from being positioned any further proximally on the staple cartridge. However, the lines of staples extend proximally beyond that position so that those staples are fired prior to the knife contacting the tissue clamped in the jaws to ensure that the cut tissue is stapled. FIG. 254 illustrates positioning of the adjunct 69400 relative to the tissue stops 60009′ of a surgical stapler. In such applications, to reduce the amount of resistance encountered by the knife as it passes through the adjunct 69400 and initially encounters the clamped tissue, it is desirable for the slot 69450 to extend distally past the tissue stops at least 5 mm (L2). This allows the knife to pass through the tissue unimpeded by the proximal end of the adjunct material 69450 to and allow for the firing of several staples through the proximal end of the adjunct 69400 before the knife starts to cut the adjunct material.

FIG. 255 illustrates an adjunct 69500 in accordance with at least one embodiment. Adjunct 69500 comprises material 69510 that may comprise any of the adjunct composition and materials described herein. The adjunct 69500 comprises a proximal end 69502 and a distal end 69508. A slot 69550 is off set from the proximal end 69502 of the adjunct. In other arrangements, the slot 69550 extends through the proximal end 69502. The slot 69550 extends completely through the thickness of the adjunct material 69510. Other embodiments are contemplated wherein the slot 69550 only extends partially through the cross-sectional thickness of the adjunct material 69510. Alternative embodiments are contemplated wherein the slot 69550 comprises a slit. The slit may extend completely through the cross-sectional thickness of the adjunct material 69510 or it may only extend partially through the cross-sectional thickness of the adjunct material 69510. In all embodiments, the slot 6950 is intended to reduce the amount of resistance encountered by the knife as it passes through the proximal end of the adjunct 69500 (in the area of likely adjunct overlap). FIG. 255 depicts the relationship of the adjunct 69500 relative to staple pockets 69518 of a staple cartridge in use. As can be seen in FIG. 255 several staple pockets 69518 are located proximal to the distal ends 60011′ of the tissue stops 60009′. Distance L3 may be at least 5 mm as described above.

FIG. 256 illustrates another adjunct 69600 arrangement that may be fabricated from an adjunct material 69610 that may comprise any of the adjunct materials and arrangements described herein. The adjunct 69600 comprises a proximal end 69602 and a distal end 69608. In this arrangement, a slot 69650 extends a distance L3 from the proximal end of the adjunct 69600, but a proximal portion 69652 of the slot 69650 is filled with another adjunct non-woven material 69654 disclosed herein that will provide less resistance to the knife than the other material making up the adjunct 69600. For example, the adjunct material 69610 comprise a woven Vicryl® material with the proximal portion 69652 of the slot 69650 filled with an oxidized regenerated cellulose (ORC) or similar material 69554. FIG. 256 illustrates the position of the slot 69650 relative to some formed staples 69618 that were formed with an anvil employing tissue stops 60009′. The slot 69650 extends a distance L4 from the proximal end 69602. In one arrangement, a distal end of the slot 69650 is at least 5 mm from the ends 60011′ of the tissue stops 60009′. In at least one embodiment, length L4 of the slot 69650 should be at least as long as the amount that a second adjunct 69600 is overlapped with a previously stapled adjunct to reduce resistance encountered by the knife as the knife travels through the area of adjunct overlap. Those of ordinary skill in the art will appreciate that any of the foregoing slot/slit arrangements may be employed with any one of the adjunct embodiments disclosed herein.

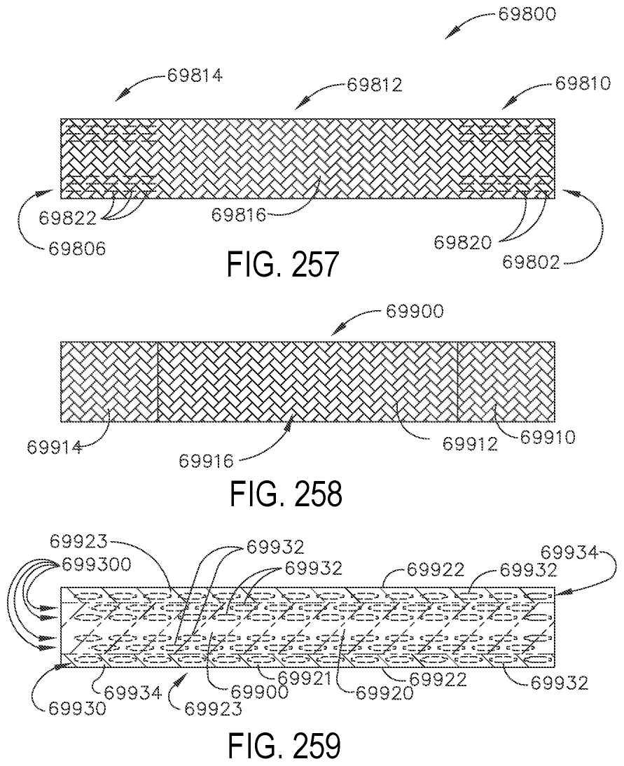

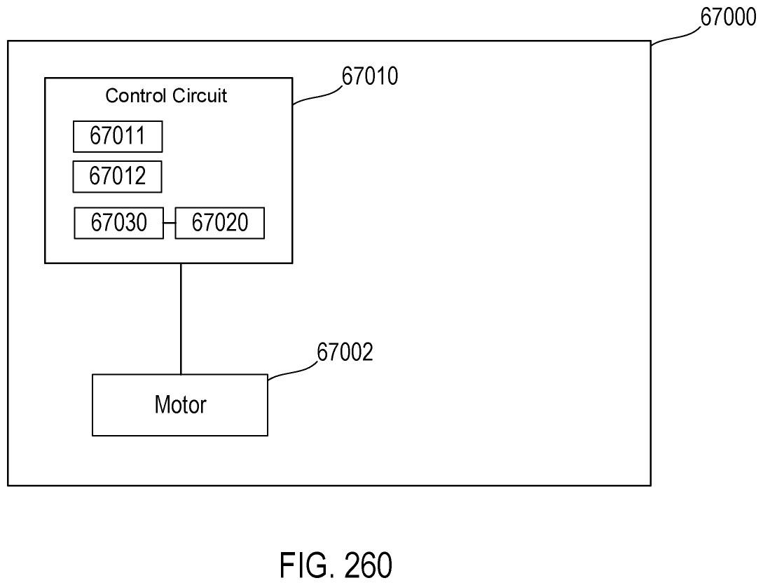

FIG. 257 depicts an adjunct 69800 that is similar to the adjuncts 69200, 69200′ 69300, and 69300′ in many instances. The adjunct 69800 comprises a woven material 69816 of the various types and constructions described herein, however the adjunct 69800 may comprise any of the non-woven adjunct materials and arrangements described herein. As can be seen in FIG. 257, the adjunct 69800 comprises a series of proximal perforations or cut out portions 69820 provided adjacent the proximal end 69802 to define a proximal weakened portion 69810 and a series of distal perforations or cut out portions 69822 adjacent a distal end 69606 to define a distal weakened portion 69814. No perforations are provided in a central portion 69812 extending between the proximal weakened portion 69810 and the distal weakened portion 69814. In use, should the adjunct 69800 start to bunch or plow during cutting, the proximal weakened portion 69810 and the distal weakened portion 69814 enable those portions to break away from the staples formed therein which may prevent those staples from being pulled out and/or damaged as the firing actuator passes therethrough. Those of ordinary skill in the art will appreciate that any of the foregoing perforation/cut out arrangements may be employed with any one of the adjunct embodiments disclosed herein including those adjuncts employing a slot or slit. In addition to the perforations and cutout portions, other embodiments comprise additionally exposing the weakened proximal portion 69810 and the weakened distal portion 69814 (but not the center portion 69812) to one or more of moisture, ultraviolet light, and radiation (gamma, X-ray, E-beam, etc.) during manufacturing and prior to use.

FIG. 258 illustrates another adjunct 69900 that comprises a woven material 69916. In one arrangement, the adjunct 69900 comprises a weakened proximal portion 69910 and a weakened distal portion 69914 which provide the above-discussed advantages. The weakened portions 69910 and 69914 are formed by exposing those portions (but not a center portion 69912) to one or more of moisture, ultraviolet light, and radiation (gamma, X-ray, E-beam, etc.) during manufacturing and prior to use. In alternative arrangements, the adjunct 69900 may comprise any of the various adjunct materials described herein with the weakened proximal portion 69910 and the weakened distal portion 69914 being created by exposing those portions of the adjunct to one or more of moisture, ultraviolet light, and radiation (gamma, X-ray, E-beam, etc.) during manufacturing and prior to use.

FIG. 259 illustrates another adjunct 69900′ that comprises a central region 69920 that corresponds to two central rows 69930 of staple pockets 69932 (shown in phantom) on each side of a longitudinal slot of a corresponding staple cartridge. The adjunct 69900′ further comprises two lateral regions 69922 and 69924 that corresponds to the two outer rows 69934 of staple pockets 69932 in the staple cartridge. In the illustrated arrangement, the central region 69920 comprises a first material 69921 and the two lateral regions 69922 comprise a second material 69923 that differs from the first material 69921. The first material 69921 comprises oxidized regenerated cellulose (ORC) or similar material and the second material comprises a woven material such as Vicryl® or similar material. The first material 69921 offers less resistance to cutting than does the second material 69923 which provides added strength to the outer rows of staples. Other materials described herein may be employed wherein the first material 69921 is weaker (offers less resistance to knife travel and/or is more likely to break away from formed staples should the adjunct start bunching or plowing) than the second material.

In one arrangement, the entire adjunct 69900′ is degraded during manufacturing by exposing the adjunct 69000′ to one or more of moisture, ultraviolet light, and radiation (gamma, X-ray, E-beam, etc.) during manufacturing and prior to use. This will further weaken the adjunct 69900′ and reduce the resistivity to cutting (and plowing) and enhance the ability to breaking away from formed staples should plowing occur during firing. In still other arrangements, the first material 69921 and/or the second material 69923 may be degraded by exposure to one or more of moisture, ultraviolet light, and radiation (gamma, X-ray, E-beam, etc.) prior to being laminated or otherwise attached together to form the adjunct 69900′.

In addition to the various attributes described above, any of the adjuncts described herein may comprise materials characterized by one or more of the following properties: biocompatible, bioabsorable, bioresorbable, biodurable, biodegradable, compressible, fluid absorbable, swellable, self-expandable, bioactive, medicament, pharmaceutically active, anti-adhesion, hemostatic, antibiotic, anti-microbial, anti-viral, nutritional, adhesive, permeable, hydrophilic and/or hydrophobic, for example. In still other configurations, the adjunct may comprise at least one of a hemostatic agent, such as fibrin and thrombin, an antibiotic, such as doxycpl, and medicament, such as matrix metalloproteinases (MMPs).

The adjuncts described herein may comprise synthetic and/or non-synthetic materials. For example, the adjunct may comprise a polymeric composition comprising one or more synthetic polymers and/or one or more non-synthetic polymers. The synthetic polymer may comprise a synthetic absorbable polymer and/or a synthetic non-absorbable polymer. The polymeric composition comprises a biocompatible foam, for example. The biocompatible foam may comprise a porous, open cell foam and/or a porous, closed cell foam, for example. The biocompatible foam may have a uniform pore morphology or may have a gradient pore morphology (i.e. small pores gradually increasing in size to large pores across the thickness of the foam in one direction). In various embodiments, the polymeric composition may comprise one or more of a porous scaffold, a porous matrix, a gel matrix, a hydrogel matrix, a solution matrix, a filamentous matrix, a tubular matrix, a composite matrix, a membranous matrix, a biostable polymer, and a biodegradable polymer, and combinations thereof. For example, the adjunct may comprise a foam reinforced by a filamentous matrix or may comprise a foam having an additional hydrogel layer that expands in the presence of bodily fluids to further provide the compression on the tissue. An adjunct described herein could also be comprised of a coating on a material and/or a second or third layer that expands in the presence of bodily fluids to further provide the compression on the tissue. Such a layer could be a hydrogel that could be a synthetic and/or naturally derived material and could be either biodurable and/or biodegradable, for example. The adjunct may comprise a microgel or a nanogel. The hydrogel may comprise carbohydrate-derived microgels and/or nanogels. An adjunct described herein may be reinforced with fibrous non-woven materials or fibrous mesh type elements, for example, that can provide additional flexibility, stiffness, and/or strength. In various embodiments, an adjunct that has a porous morphology which exhibits a gradient structure such as, for example, small pores on one surface and larger pores on the other surface is employed. Such morphology could be more optimal for tissue in-growth or hemostatic behavior. Further, the gradient could be also compositional with a varying bio-absorption profile. A short term absorption profile may be preferred to address hemostasis while a long term absorption profile may address better tissue healing without leakages, for example.

Examples of non-synthetic materials that may comprise any of the adjuncts described herein include, but are not limited to, lyophilized polysaccharide, glycoprotein, bovine pericardium, collagen, gelatin, fibrin, fibrinogen, elastin, proteoglycan, keratin, albumin, hydroxyethyl cellulose, cellulose, oxidized cellulose, oxidized regenerated cellulose (ORC), hydroxypropyl cellulose, carboxyethyl cellulose, carboxymethylcellulose, chitan, chitosan, casein, alginate, and combinations thereof.

Examples of synthetic absorbable materials that may comprise any of the adjuncts disclosed herein include, but are not limited to, poly(lactic acid) (PLA), poly(L-lactic acid) (PLLA), polycaprolactone (PCL), polyglycolic acid (PGA), poly(trimethylene carbonate) (TMC), polyethylene terephthalate (PET), polyhydroxyalkanoate (PHA), a copolymer of glycolide and ε-caprolactone (PGCL), a copolymer of glycolide and -trimethylene carbonate, poly(glycerol sebacate) (PGS), poly(dioxanone) (PDS), polyesters, poly(orthoesters), polyoxaesters, polyetheresters, polycarbonates, polyamide esters, polyanhydrides, polysaccharides, poly(ester-amides), tyrosine-based polyarylates, polyamines, tyrosine-based polyiminocarbonates, tyrosine-based polycarbonates, poly(D,L-lactide-urethane), poly(hydroxybutyrate), poly(B-hydroxybutyrate), poly(E-caprolactone), polyethyleneglycol (PEG), poly[bis(carboxylatophenoxy)phosphazene]poly(amino acids), pseudo-poly(amino acids), absorbable polyurethanes, poly(phosphazine), polyphosphazenes, polyalkyleneoxides, polyacrylamides, polyhydroxyethylmethylacrylate, polyvinylpyrrolidone, polyvinyl alcohols, poly(caprolactone), polyacrylic acid, polyacetate, polypropylene, aliphatic polyesters, glycerols, copoly(ether-esters), polyalkylene oxalates, polyamides, poly(iminocarbonates), polyalkylene oxalates, and combinations thereof. In various embodiments, the polyester is may be selected from the group consisting of polylactides, polyglycolides, trimethylene carbonates, polydioxanones, polycaprolactones, polybutesters, and combinations thereof.

In various embodiments, any of the adjuncts described herein may comprise a surfactant. Examples of surfactants may include, but are not limited to, polyacrylic acid, methalose, methyl cellulose, ethyl cellulose, propyl cellulose, hydroxy ethyl cellulose, carboxy methyl cellulose, polyoxyethylene cetyl ether, polyoxyethylene lauryl ether, polyoxyethylene octyl ether, polyoxyethylene octylphenyl ether, polyoxyethylene oleyl ether, polyoxyethylene sorbitan monolaurate, polyoxyethylene stearyl ether, polyoxyethylene nonylphenyl ether, dialkylphenoxy poly(ethyleneoxy) ethanol, and polyoxamers.

In various embodiments, the polymeric composition comprises a pharmaceutically active agent. The polymeric composition may release a therapeutically effective amount of the pharmaceutically active agent. The pharmaceutically active agent may be released as the polymeric composition is desorbed/absorbed. In various embodiments, the pharmaceutically active agent may be released into fluid, such as, for example, blood, passing over or through the polymeric composition. Examples of pharmaceutically active agents may include, but are not limited to, hemostatic agents and drugs, such as, for example, fibrin, thrombin, and oxidized regenerated cellulose (ORC); anti-inflammatory drugs, such as, for example, diclofenac, aspirin, naproxen, sulindac, and hydrocortisone; antibiotic and antimicrobial drug or agents, such as, for example, triclosan, ionic silver, ampicillin, gentamicin, polymyxin B, chloramphenicol; and anticancer agents, such as, for example, cisplatin, mitomycin, adriamycin.

In various embodiments, the polymeric composition comprises a hemostatic material. The hemostatic material may comprise poly(lactic acid), poly(glycolic acid), poly(hydroxybutyrate), poly(caprolactone), poly(dioxanone), polyalkyleneoxides, copoly(ether-esters), collagen, gelatin, thrombin, fibrin, fibrinogen, fibronectin, elastin, albumin, hemoglobin, ovalbumin, polysaccharides, hyaluronic acid, chondroitin sulfate, hydroxyethyl starch, hydroxyethyl cellulose, cellulose, oxidized cellulose, hydroxypropyl cellulose, carboxyethyl cellulose, carboxymethyl cellulose, chitan, chitosan, agarose, maltose, maltodextrin, alginate, clotting factors, methacrylate, polyurethanes, cyanoacrylates, platelet agonists, vasoconstrictors, alum, calcium, RGD peptides, proteins, protamine sulfate, ¿-amino caproic acid, ferric sulfate, ferric subsulfates, ferric chloride, zinc, zinc chloride, aluminum chloride, aluminum sulfates, aluminum acetates, permanganates, tannins, bone wax, polyethylene glycols, fucans and combinations thereof. In one form, the adjunct is characterized by hemostatic properties.

As discussed herein, various surgical instruments comprise a motor control system 67000 configured to execute intelligent algorithms. The intelligent algorithms comprise sequences that are optimized to interact with physical features of an adjunct. The motor control system 67000 may detect the changes in the adjunct thickness and/or properties and adapt the firing stroke in response to the detected changes. In some instances, the motor control system 67000 can change the firing speed and/or pause at locations in the adjunct if a different thickness and/or different properties are detected based on a predetermined initial threshold.

The motor control system 67000 comprises a control circuit 67010. The control circuit 67010 comprises a processor 67011 and a memory device 67012. The processor 67011 is in communication with the motor 67002 of the surgical instrument. The motor control system 67000 can include and/or receive data 67020 regarding the adjunct loaded in the instrument and adapt the firing stroke in response to the data 67020. In some instances the data 67020 comprises a predetermined adjunct thickness threshold 67022. The data 67020 may be stored on a memory device 67030 within the control circuit 67010. The data 67020 may also be stored in or on the cartridge itself. In some instances, the cartridge 60026 comprises an RFID tag 67040 affixed thereto.

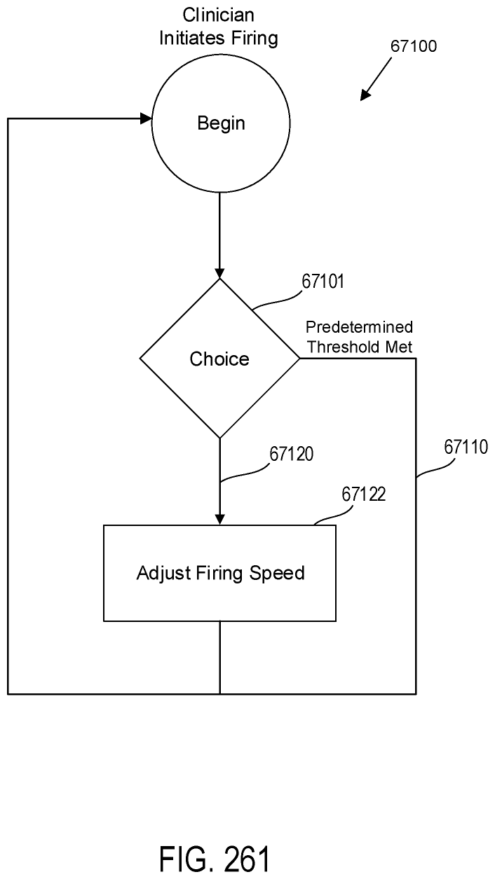

In various instances, the surgical instruments described herein comprise a control board, such as a printed control board (PCB), for example, which comprises the hardware and software for the motor control system 67000 of the surgical instruments. When the clinician initiates a firing motion, the firing member begins traveling distally through the cartridge which is detected by the motor control system 67000. At this point, the motor control system 67000 follows an algorithm 67100 for deciding when, or if, to adjust the firing speed. An algorithm 67100 is illustrated in FIG. 261 which can control this, although any suitable algorithm could be used.

As discussed above, the motor control system of a surgical instrument can comprise an intelligent algorithm 67100 which, according to predetermined criteria such as physical features of the adjunct, changes the firing speed or pauses the firing motion of the surgical instrument in certain instances. In various instances, as also discussed above, the algorithm can be configured to modify aspects of the firing motion of the surgical instrument based on the predetermined adjunct thickness threshold. As illustrated in FIG. 261, the algorithm 67100 comprises a step 67101 in which the motor control system 67000 assesses whether or not the physical features of the adjunct affixed to the staple cartridge aligns with the predetermined initial threshold. If it is determined at step 67101 that the physical features of the adjunct align with the predetermined initial threshold, the algorithm 67100 follows logic path 67110. In logic path 67110, the motor control system 67000 operates the surgical instrument at a consistent firing speed. Alternatively, if at step 67101 the motor control system 67000 determines that the physical features of the adjunct do not align with the predetermined initial threshold, the algorithm follows logic path 67120. In logic path 67120, the motor control system 67000 increases or decreases the speed of the firing motion at step 67122 to accommodate an adjunct that is thicker or thinner than the predetermined initial threshold, for example.

Further to the above, the motor control system 67000 of the surgical instrument 60000 comprises a pulse width modulation (PWM) control circuit configured to control the speed of the firing drive electric motor. The PWM control circuit applies voltage pulses to the firing drive electric motor to perform the staple firing stroke. In various instances, the PWM control circuit increases the duration of the voltage pulses it applies to the firing drive electric motor in order to increase the speed of the firing drive electric motor and, correspondingly, the speed of the staple firing stroke. In other instances, the PWM control circuit decreases the duration of the voltage pulses it applies to the firing drive electric motor in order to decrease the speed of the firing drive electric motor and, correspondingly, the speed of the staple firing stroke. In either event, the PWM control circuit can make these pulse length adjustments without substantially increasing or decreasing the magnitude of the voltage pulses being applied to the motor. That said, embodiments are envisioned in which the magnitude of the voltage pulses, or certain voltage pulses, could be changed. In any event, as described in greater detail below, the control system is configured to drive the staple firing drive at a constant, or near constant, speed by adjusting the duration of the pulses via the PWM circuit. The entire disclosure of U.S. Pat. No. 8,499,992, entitled DEVICE AND METHOD FOR CONTROLLING COMPRESSION OF TISSUE, which issued on Aug. 6, 2013, is incorporated by reference herein.

The ratio of the time in which the voltage is applied to the electric motor (ON time) by the PWM circuit divided by the total time (ON time+OFF time) is the duty cycle of the staple firing drive motor. Thus, the duty cycle can range between 0% (completely OFF) and 100% (completely ON), i.e., a constant voltage without periodic interruptions. The terms ON and OFF suggest a non-zero voltage and a zero voltage; however, the terms ON and OFF are inclusive of HIGH and LOW voltages, respectively. The terms LOW or OFF include zero voltage and non-zero voltages that have a magnitude which is less than the HIGH or ON voltage. In view of the above, another way of expressing the duty cycle of the firing drive electric motor is the ratio of the time in which the voltage is applied to the electric motor (HIGH time) by the PWM circuit divided by the total time (HIGH time+LOW time).

The PWM control circuit applies the voltage pulses to the firing drive electric motor at regular intervals; however, the control system can comprise a frequency modulation (FM) control circuit to change the frequency of the voltage pulse intervals. In various instances, the FM control circuit decreases the interval between the voltage pulses to increase the speed of the firing drive electric motor and the staple firing stroke. Correspondingly, the FM control circuit increases the interval between the voltage pulses to decrease the speed of the firing drive electric motor and the staple firing stroke. In addition to or in lieu of the above, the control system can increase the magnitude of the voltage it applies to the firing drive electric motor to increase the speed of the firing drive electric motor and the staple firing stroke and/or decrease the magnitude of the voltage it applies to the firing drive electric motor to decrease the speed of the firing drive electric motor and the staple firing stroke.

The control system of the surgical instrument 60000 comprises an algorithm for controlling the speed of the staple firing member. The motor control system 67000 can included another algorithm configured to drive the staple firing member at a low speed, an intermediate speed, and a high speed. The low speed is 6 mm/s, or approximately 6 mm/s. The intermediate speed is 12 mm/s, or approximately 12 mm/s. The high speed is 20 mm/s, or approximately 20 mm/s. That said, the motor control system 67000 can be configured to operate the surgical instrument at any suitable number of speeds and/or at any suitable speed. The control system is configured to monitor the speed of the staple firing drive, via a motor speed sensor, and adjust the length of the voltage pulses applied to the electric motor of the staple firing drive to bring the speed of the staple firing drive to the target speed. For instance, if the target speed of the staple firing drive at a given point in the staple firing stroke is 12 mm/s and the actual speed is 11 mm/s, the control system increases the length of the voltage pulses it is applying to the electric motor to increase the speed of the staple firing drive. Stated another way, the control system increases the duty cycle of the firing drive electric motor to increase the speed of the staple firing drive. Correspondingly, the motor control system 67000 is configured to shorten the length of the voltage pulses it is applying to the firing drive electric motor if the speed of the staple firing drive exceeds the target speed until the speed of the staple firing drive reaches the target speed. Stated another way, the control system is configured to lower the duty cycle of the firing drive electric motor to decrease the speed of the staple firing drive. Notably, the target speed for the staple firing drive can change during the staple firing stroke. The entire disclosure of U.S. patent application Ser. No. 17/728,089, entitled STAPLING INSTRUMENT COMPRISING JAW MOUNTS, is incorporated by reference herein.

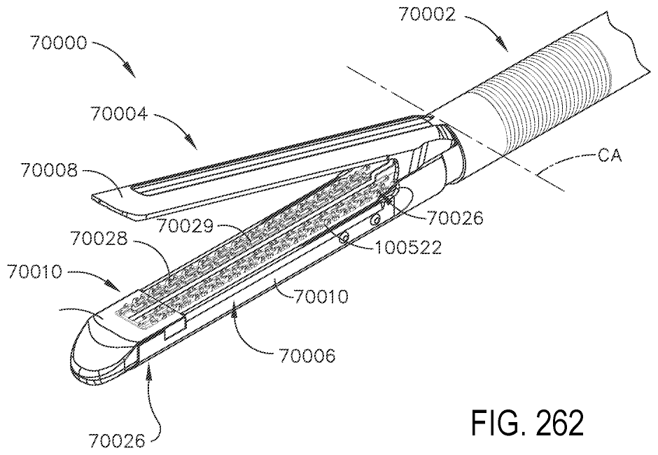

FIG. 262 depicts a surgical stapler 70000 comprising a shaft 70002 that includes an end effector 70004 that extends from the shaft 70002. The end effector 70004 comprises a first jaw 70006 and a second jaw 70008. The first jaw 70006 comprises an elongate channel 70010. A staple cartridge 70020 is insertable into and removable from the elongate channel 70010. Other embodiments are envisioned in which the staple cartridge 70020 is not removable from, or at least readily replaceable from, the first jaw 70006. The second jaw 70008 comprises an anvil 70030 that is configured to form staples that are ejected from the staple cartridge 70020. The second jaw 70008 is pivotable relative to the first jaw 70006 between an open position and a closed position; however, other embodiments are envisioned in which the first jaw 70006 is pivotable relative to the second jaw 70008.

The staple cartridge 70020 includes a cartridge body 70022 that comprises a proximal end 70024 and a distal end 70026. The cartridge body 70022 further comprises a deck surface 70028 that extends between the proximal end 70024 and the distal end 70026 and is configured to oppose the anvil 70030. A longitudinal slot 70029 extends from the proximal end 70024 toward the distal end 70026 of the cartridge body 70022 and is sized to receive a sled driver or firing actuator that is configured to eject staples out of the cartridge body 70022 during a cutting and staple firing stroke. Various aspects of staple cartridges are described in greater detail in U.S. Pat. No. 9,844,369, the disclosure of which is herein incorporated by reference in its entirety.

Many surgical applications require the use of buttresses or tissue thickness compensators that are commonly referred to as adjuncts to be used in conjunction with the stapling end effectors described herein in order to reinforce a staple line and provide support to the tissue surrounding the staple line. Various adjuncts are disclosed herein and may comprise materials characterized by one or more of the following properties: biocompatible, bioabsorable, bioresorbable, biodurable, biodegradable, compressible, fluid absorbable, swellable, self-expandable, bioactive, medicament, pharmaceutically active, anti-adhesion, hemostatic, antibiotic, anti-microbial, anti-viral, nutritional, adhesive, permeable, hydrophilic and/or hydrophobic, for example. Any of the adjuncts disclosed herein may comprise at least one of a hemostatic agent, such as fibrin and thrombin, an antibiotic, such as doxycpl, and medicament, such as matrix metalloproteinases (MMPs).

The various adjuncts disclosed herein may comprise synthetic and/or non-synthetic materials. An adjunct may comprise a polymeric composition comprising one or more synthetic polymers and/or one or more non-synthetic polymers. The synthetic polymer may comprise a synthetic absorbable polymer and/or a synthetic non-absorbable polymer. In various embodiments, the polymeric composition may comprise a biocompatible foam, for example. The biocompatible foam may comprise a porous, open cell foam and/or a porous, closed cell foam, for example. The biocompatible foam may have a uniform pore morphology or may have a gradient pore morphology (i.e. small pores gradually increasing in size to large pores across the thickness of the foam in one direction). The polymeric composition may comprise one or more of a porous scaffold, a porous matrix, a gel matrix, a hydrogel matrix, a solution matrix, a filamentous matrix, a tubular matrix, a composite matrix, a membranous matrix, a biostable polymer, and a biodegradable polymer, and combinations thereof. For example, the adjunct may comprise a foam reinforced by a filamentous matrix or may comprise a foam having an additional hydrogel layer that expands in the presence of bodily fluids to further provide the compression on the tissue. Any of the adjuncts disclosed herein could also be comprised of a coating on a material and/or a second or third layer that expands in the presence of bodily fluids to further provide the compression on the tissue. Such a layer could be a hydrogel that could be a synthetic and/or naturally derived material and could be either biodurable and/or biodegradable, for example. In various embodiments, the adjunct may comprise a microgel or a nanogel. The hydrogel may comprise carbohydrate-derived microgels and/or nanogels. In certain embodiments, an adjunct may be reinforced with fibrous non-woven materials or fibrous mesh type elements, for example, that can provide additional flexibility, stiffness, and/or strength. In various embodiments, an adjunct that has a porous morphology which exhibits a gradient structure such as, for example, small pores on one surface and larger pores on the other surface. Such morphology could be more optimal for tissue in-growth or hemostatic behavior. Further, the gradient could be also compositional with a varying bio-absorption profile. A short-term absorption profile may be preferred to address hemostasis while a long-term absorption profile may address better tissue healing without leakages.

Examples of non-synthetic materials from which an adjunct disclosed herein may be comprised of include, but are not limited to, lyophilized polysaccharide, glycoprotein, bovine pericardium, collagen, gelatin, fibrin, fibrinogen, elastin, proteoglycan, keratin, albumin, hydroxyethyl cellulose, cellulose, oxidized cellulose, oxidized regenerated cellulose (ORC), hydroxypropyl cellulose, carboxyethyl cellulose, carboxymethylcellulose, chitan, chitosan, casein, alginate, and combinations thereof.

Examples of synthetic absorbable materials include, but are not limited to, poly(lactic acid) (PLA), poly(L-lactic acid) (PLLA), polycaprolactone (PCL), polyglycolic acid (PGA), poly(trimethylene carbonate) (TMC), polyethylene terephthalate (PET), polyhydroxyalkanoate (PHA), a copolymer of glycolide and ε-caprolactone (PGCL), a copolymer of glycolide and -trimethylene carbonate, poly(glycerol sebacate) (PGS), poly(dioxanone) (PDS), polyesters, poly(orthoesters), polyoxaesters, polyetheresters, polycarbonates, polyamide esters, polyanhydrides, polysaccharides, poly(ester-amides), tyrosine-based polyarylates, polyamines, tyrosine-based polyiminocarbonates, tyrosine-based polycarbonates, poly(D,L-lactide-urethane), poly(hydroxybutyrate), poly(B-hydroxybutyrate), poly(E-caprolactone), polyethyleneglycol (PEG), poly[bis(carboxylatophenoxy)phosphazene]poly(amino acids), pseudo-poly(amino acids), absorbable polyurethanes, poly(phosphazine), polyphosphazenes, polyalkyleneoxides, polyacrylamides, polyhydroxyethylmethylacrylate, polyvinylpyrrolidone, polyvinyl alcohols, poly(caprolactone), polyacrylic acid, polyacetate, polypropylene, aliphatic polyesters, glycerols, copoly(ether-esters), polyalkylene oxalates, polyamides, poly(iminocarbonates), polyalkylene oxalates, and combinations thereof. In various embodiments, the polyester is may be selected from the group consisting of polylactides, polyglycolides, trimethylene carbonates, polydioxanones, polycaprolactones, polybutesters, and combinations thereof.

In various embodiments, the adjunct may comprise a surfactant. Examples of surfactants may include, but are not limited to, polyacrylic acid, methalose, methyl cellulose, ethyl cellulose, propyl cellulose, hydroxy ethyl cellulose, carboxy methyl cellulose, polyoxyethylene cetyl ether, polyoxyethylene lauryl ether, polyoxyethylene octyl ether, polyoxyethylene octylphenyl ether, polyoxyethylene oleyl ether, polyoxyethylene sorbitan monolaurate, polyoxyethylene stearyl ether, polyoxyethylene nonylphenyl ether, dialkylphenoxy poly(ethyleneoxy) ethanol, and polyoxamers.

In various embodiments, the polymeric composition may comprise a pharmaceutically active agent. The polymeric composition may release a therapeutically effective amount of the pharmaceutically active agent. In various embodiments, the pharmaceutically active agent may be released as the polymeric composition is desorbed/absorbed. In various embodiments, the pharmaceutically active agent may be released into fluid, such as, for example, blood, passing over or through the polymeric composition. Examples of pharmaceutically active agents may include, but are not limited to, hemostatic agents and drugs, such as, for example, fibrin, thrombin, and oxidized regenerated cellulose (ORC); anti-inflammatory drugs, such as, for example, diclofenac, aspirin, naproxen, sulindac, and hydrocortisone; antibiotic and antimicrobial drug or agents, such as, for example, triclosan, ionic silver, ampicillin, gentamicin, polymyxin B, chloramphenicol; and anticancer agents, such as, for example, cisplatin, mitomycin, adriamycin.

In various embodiments, the polymeric composition may comprise a hemostatic material. The adjunct may comprise hemostatic materials comprising poly(lactic acid), poly(glycolic acid), poly(hydroxybutyrate), poly(caprolactone), poly(dioxanone), polyalkyleneoxides, copoly(ether-esters), collagen, gelatin, thrombin, fibrin, fibrinogen, fibronectin, elastin, albumin, hemoglobin, ovalbumin, polysaccharides, hyaluronic acid, chondroitin sulfate, hydroxyethyl starch, hydroxyethyl cellulose, cellulose, oxidized cellulose, hydroxypropyl cellulose, carboxyethyl cellulose, carboxymethyl cellulose, chitan, chitosan, agarose, maltose, maltodextrin, alginate, clotting factors, methacrylate, polyurethanes, cyanoacrylates, platelet agonists, vasoconstrictors, alum, calcium, RGD peptides, proteins, protamine sulfate, ¿-amino caproic acid, ferric sulfate, ferric subsulfates, ferric chloride, zinc, zinc chloride, aluminum chloride, aluminum sulfates, aluminum acetates, permanganates, tannins, bone wax, polyethylene glycols, fucans and combinations thereof. The adjunct may be characterized by hemostatic properties.

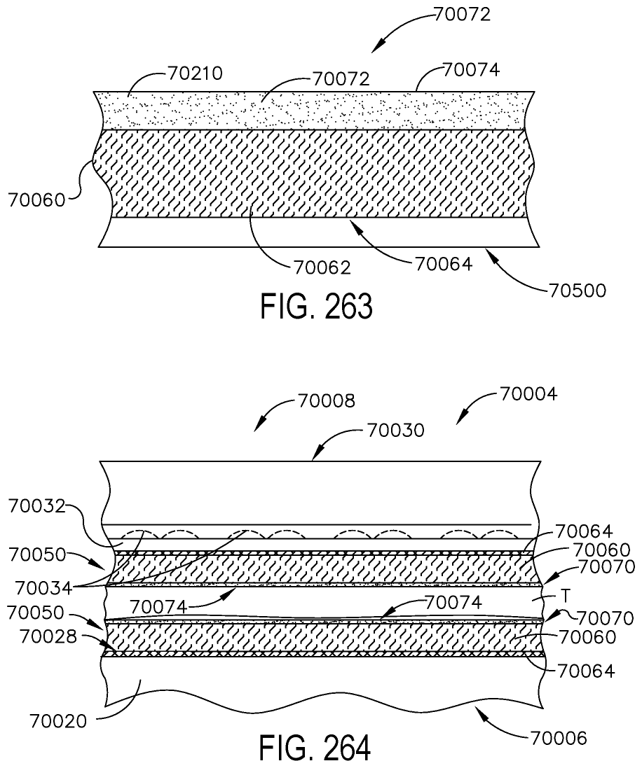

FIG. 263 illustrates an adjunct 70050 in accordance with at least one embodiment. The adjunct 70050 comprises a first material layer 70060 that, in at least one arrangement, comprises a Vicryl® mesh material 70062. The Vicryl® mesh material provides a woven-type of support structure for supporting the surgical staples as well as the severed tissue when fastened thereto. The first material layer 70060 may alternatively comprise other mesh or woven materials described herein. In the illustrated arrangement, the first material layer 70060 defines a first device-contacting surface 70064. The adjunct 70050 further comprises a second film layer 70070 that is adhered to a side of the first material layer 70060 that is opposite to the device-contacting surface 70064. In one arrangement, the second film layer 70070 is formed from a second material 70072 that differs from the first material 70062 and comprises a polydioxanone (PDO) film that helps to further stabilize the first mesh material 70062. In the illustrated arrangement, the second film layer 70070 defines a planar or substantially planar tissue-contacting surface 70074. The tissue-contacting surface 70074 is smooth as compared to the mesh, woven or lattice device-contacting surface 70064. In alternative arrangements, the second film layer 70070 may be formed from alternative synthetic polymer materials described herein.

As can be seen in FIG. 264, in use, the adjunct 70050 may be positioned between the jaws 70006, 70008 of a surgical stapler end effector 70004 that comprises a surgical staple cartridge 70020 and an anvil 70030, respectively. In applications wherein only one adjunct 70050 is employed, the device-contacting surface 70064 is either placed in contact with a staple-forming undersurface 70032 of the anvil 70030 or a deck surface 70028 on the staple cartridge 70020. Those of ordinary skill in the art will appreciate that the mesh, woven or lattice configuration of the device-contacting surface 70064 will serve to establish a frictional interface with the staple-forming undersurface 70032 of the anvil 70030 or the deck surface 70028 of the staple cartridge 70020 to prevent the adjunct 70050 from slipping or otherwise moving laterally and/or longitudinally when initially contacted by the knife of the firing actuator or sled driver of the surgical stapler. FIG. 264 illustrates an application wherein two adjuncts 70050 are employed. As can be seen in FIG. 264, when two adjuncts 70050 are employed, the device-contacting surface 70064 of one adjunct 70050 is placed in contact with the staple-forming undersurface 70032 of the anvil 70030 and the device-contacting surface 70064 of the other adjunct 70050 is placed in contact with the deck surface 70028 of the staple cartridge 70020. The target tissue T is then clamped between the tissue-contacting surfaces 70074 of each of the adjuncts 70050.

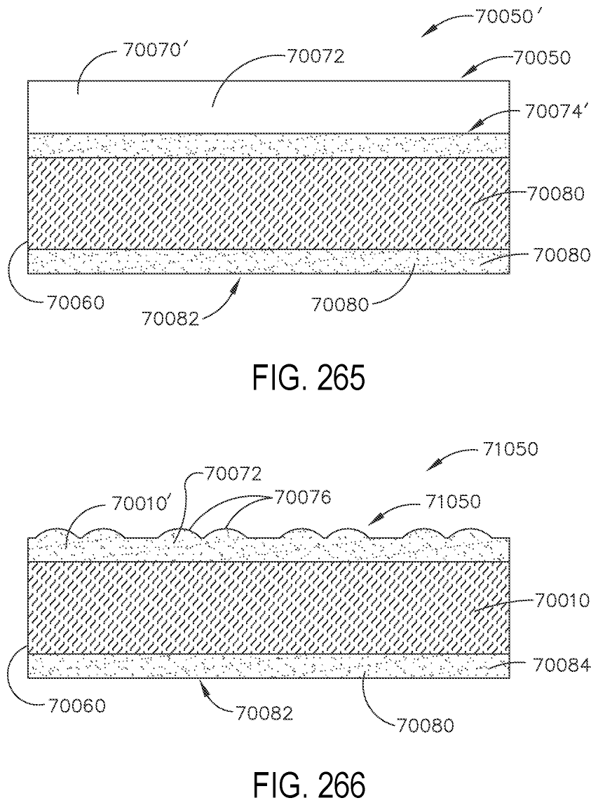

FIG. 265 depicts another adjunct 70050′ that is somewhat similar to adjunct 70050. As can be seen in FIG. 265, adjunct 70050′ additionally comprises a third film layer 70080 that defines a tissue-contacting surface 70082. The third film layer comprises a third material 70084 which, in at least one arrangement, is the same as the second material 70072. That is, the third material 70084 also comprises polydioxanone (PDO) film. The first film layer 70060 and the third film layer 70080 may be applied to the second material layer in one operation such that the second material layer is completely encapsulated in the polydioxanone (PDO) film. In this embodiment, the second film layer 70070′ defines a planar or substantially planar textured or roughened device-contacting surface 70074′.

In use, the adjunct 70050′ may be positioned between the jaws of a surgical stapler end effector that comprises an anvil and a surgical staple cartridge. In applications wherein only one adjunct 70050′ is employed, the device-contacting surface 70074′ is either placed in contact with the staple-forming undersurface of the anvil or the deck surface of the staple cartridge. Those of ordinary skill in the art will appreciate that the textured or roughened surface 70074′ will serve to establish a frictional interface with the anvil or staple cartridge to prevent the adjunct 70050′ from slipping or moving when initially contacted by the knife of the firing actuator or sled driver of the surgical stapler. In applications wherein two adjuncts 70050′ are employed, the device-contacting surface 70074′ of one adjunct 70050′ is placed in contact with the staple-forming surface of the anvil and the device-contacting surface 70074′ of the other adjunct 70050′ is placed in contact with the deck surface of the staple cartridge. The target tissue is then clamped between the tissue-contacting surfaces 70074′ of each of the adjuncts 70050′. The layered adjunct 70050′ enjoys the advantages provided by the woven material 70062 as well as the advantages provided by the non-woven material(s) 70072, 70084 while also minimizing the likelihood of the adjunct 70050′ moving, plowing or bunching during cutting and firing.

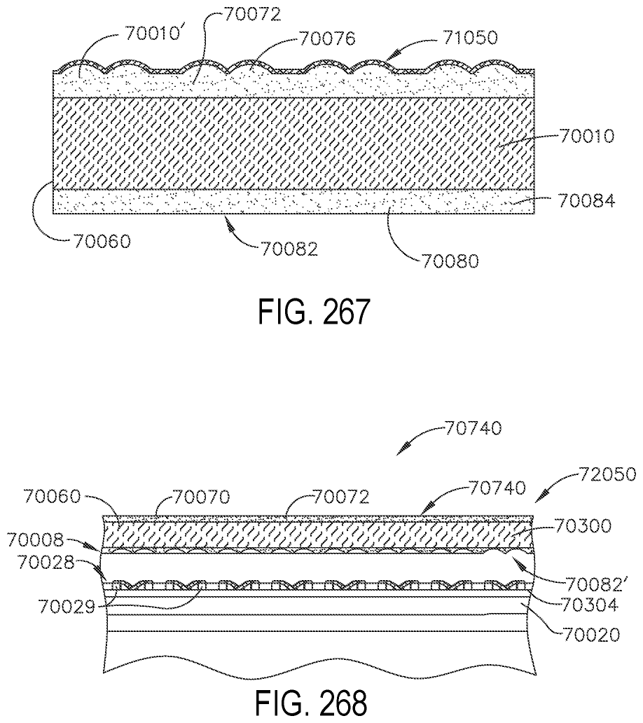

FIG. 266 illustrates another adjunct 71050 that is similar to the adjunct 70050′ described above, except that the device-contacting surface 71074 of the second film layer 70070′ is contoured to frictionally engage the staple-forming undersurface 70032 of the anvil 70030. For example, the device-contacting surface 71074 comprises a plurality of convex friction bumps 71076 that correspond to the staple-forming pockets 70034 in the anvil 70030. In use, the adjunct 71050 is placed adjacent to the anvil so that the friction bumps 71076 frictionally engage corresponding staple-forming pockets 70034 in the anvil 70030. Such arrangement serves to prevent the adjunct 71050 from slipping or moving when initially contacted by the knife of the firing actuator or sled driver of the surgical stapler. In an alternative arrangement, the contoured device-contacting surface may also comprise a continuous roughened or textured surface 70078 to further enhance the frictional engagement between the adjunct 71050 and the anvil 70030. See FIG. 267.

FIG. 268 depicts an adjunct 72050 that is similar to adjunct 70050′ except that the device-contacting surface 70082′ of the third film layer 70080′ is contoured to frictionally engage the deck 70028 of the surgical staple cartridge 70020. As can be seen in FIG. 268, the deck surface 70028 of the staple cartridge 70020 additionally comprises a plurality of features or protrusions 70029 that correspond to the staple pockets in the staple cartridge 70020. In the illustrated arrangement, the device-contacting surface 70082′ has a plurality of concave cavities 70086 that correspond to the protrusions 70029 on the staple cartridge 70020. Such arrangement serves to prevent the adjunct 72050 from slipping or moving when initially contacted by the knife of the firing actuator or sled driver of the surgical stapler. In an alternative arrangement, the contoured device-contacting surface 70082′ may also comprise a roughened or textured surface to further enhance the frictional engagement between the adjunct 72050 and the deck 70028 of the staple cartridge 70020.

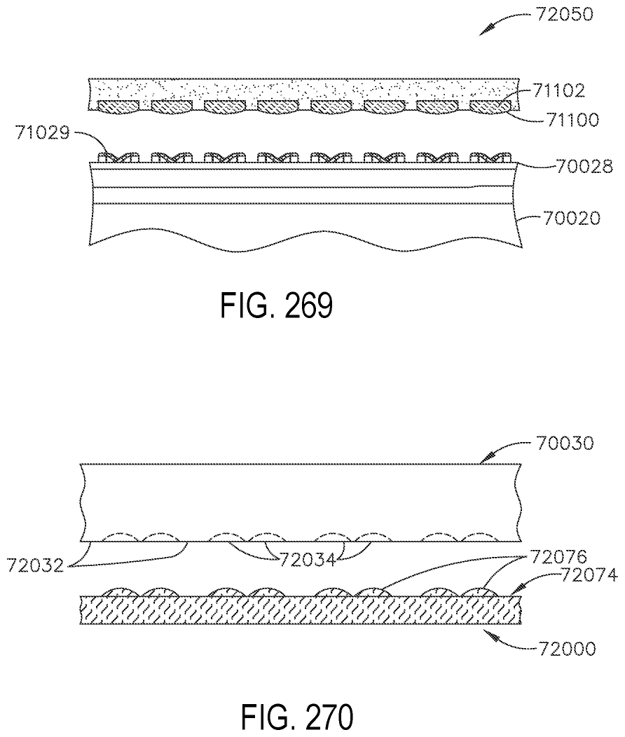

An adjunct 72050′ and a staple cartridge 70020 are illustrated in FIG. 269. The staple cartridge 70020 comprises a cartridge deck 70028 and cartridge deck features 70029. The adjunct 72050′ comprises a layer of first material 72060 that contains a pattern of traction bumps 71100. The first material 72060 may comprise any of the various adjunct materials disclosed herein. In one arrangement, the first layer comprises polydioxanone (PDO) or other synthetic polymer material disclosed herein. The traction bumps 71100 are fabricated from a second material 71102 that is embedded into the first material 72060 and has a coefficient of friction that is greater than the coefficient of friction of the first material 72060. For example, the second material may comprise a Vicryl® mesh. The traction bumps 71100 are configured to contact the cartridge deck features 71220. The traction bumps 71100 of the adjunct 71000 are configured to reduce slipping or sliding of the adjunct 72050′ relative to the staple cartridge 70020 and staple cartridge deck 70028.

FIG. 270 illustrates another adjunct 72000 is formed from a single material 72002. In one arrangement, for example, the material 72002 comprises polydioxanone (PDO) or other synthetic polymer material disclosed herein. The adjunct 72000 defines a device-contacting surface 72074 comprises a plurality of convex friction bumps 72076 that correspond to the staple-forming pockets 70034 in the anvil 70030. In use, the adjunct 72000 is placed adjacent to the anvil 70030 so that the friction bumps 72076 frictionally engage corresponding staple-forming pockets 70034 in the anvil 70030. Such arrangement serves to prevent the adjunct 72000 from slipping or moving when initially contacted by the knife of the firing actuator or sled driver of the surgical stapler. In an alternative arrangement, the contoured device-contacting surface may also comprise a roughened or textured surface to further increase the frictional engagement between the adjunct 72000 and the anvil 70030.

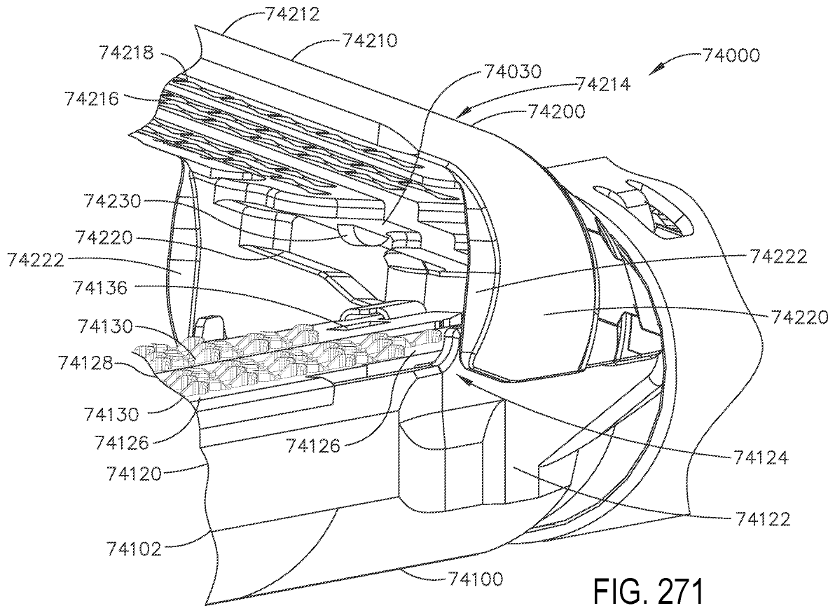

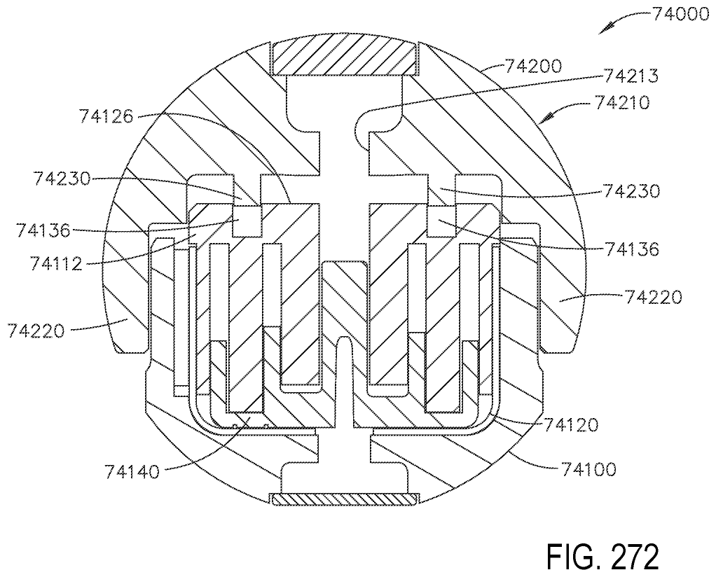

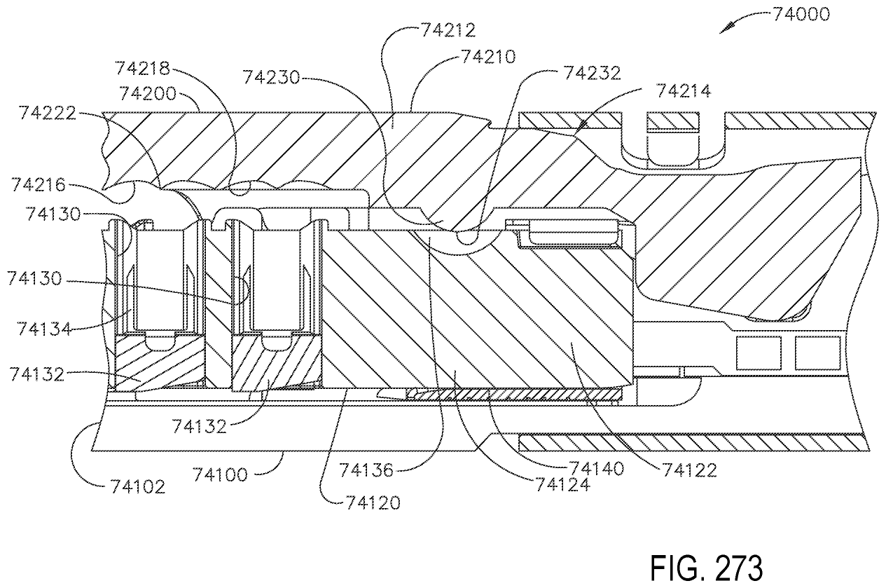

A surgical stapler end effector 74000 in accordance with one embodiment is illustrated in FIGS. 271-274. The end effector 74000 comprises a first jaw 74100 and a second jaw 74200. The first jaw 74100 comprises an elongate channel 74102 that is configured to support a surgical staple cartridge 74120 therein. The staple cartridge 74120 is insertable into and removable from the elongate channel 74102; however, other embodiments are envisioned in which the staple cartridge 74120 is not removable from, or at least readily replaceable from, the elongate channel 74102. The staple cartridge 74120 comprises a cartridge body 74122 that has a proximal end portion 74124 and a distal end portion (not shown). The cartridge body 74122 defines a deck surface 74126 that extends from the proximal end portion 74124 to the distal end portion of the cartridge body 74122. An elongate slot 74128 extends from the proximal end portion 74214 to the distal end portion to accommodate passage of a sled 74140 (and sled driver or firing actuator of the surgical stapling instrument) therethrough. See FIG. 272. In at least one arrangement, three longitudinal lines of lines staple cavities 74130 are provided on each side of the elongate slot 74128. A corresponding staple driver 74132 is movably supported in each staple cavity 74130. One or more surgical staples 74134 or fasteners are removably supported on each driver 74132. See FIG. 273. Other staple cartridge arrangements and fastener arrangements are contemplated wherein the each fastener has an integral driver formed therewith. In any event, as the sled 74140 is driven distally by a sled driver or firing actuator of the surgical stapling instrument, the sled 74140 sequentially contacts the drivers 74132 in each longitudinal row and drives the drivers and the staples 74134 or fasteners supported thereon out of the corresponding staple cavity.

The second jaw 74200 comprises an anvil 74210 which is configured to deform staples 74134 ejected from the staple cartridge 74120. The anvil 74210 comprises an anvil body 74212 that has a proximal end portion 74214 and a distal end portion (not shown). A staple-forming surface 74216 extends from the proximal end portion 74214 to the distal end portion and defines a plurality of staple-forming pockets 74218 therein. An elongate anvil slot 74213 extends from the proximal end portion 74214 to the distal end portion to accommodate passage of the sled driver or firing actuator. The anvil 74210 further comprises a pair of tissue stops 74220 that protrude downward on each side of the proximal end portion 74214. The tissue stops 74220 each define a distal edge 74222 that is configured to be contacted by the target tissue when the target tissue is positioned between the anvil and the staple cartridge to prevent the target tissue from being positioned proximally beyond the proximal-most staples in the staple cartridge 74120.

The anvil 74210 further comprises at least one adjunct retainer in the form of a protrusion, tooth, or dimple 74230 that is formed on the proximal end portion 74214 and protrudes downwardly toward the staple cartridge 74120. As can be seen in FIG. 272, in the illustrated embodiment, one protrusion 74230 is formed on one side of the anvil slot 74213 and another protrusion 74230 is formed on the other side of the anvil slot 74213. As can be seen in FIG. 273, each protrusion 74230 comprises a rounded surface 74232 that corresponds with a concave cavity, aperture or hole 74136 provided in the proximal end 74124 of the staple cartridge 74120. The rounded surface 74232 reduces the chance of tissue damage or trauma should tissue be inadvertently captured between the proximal end portion 74214 of the anvil 74210 and the proximal end 74122 of the staple cartridge 74120. However, as can be seen in FIG. 273, in at least one arrangement, the protrusions 74230 are proximal to the distal edges 74222 of the tissue stops 74220 as well as proximal to the proximal-most staple cavities 74130 and staples 74132 in the staple cartridge 74120. Thus, the tissue stops 74120 will prevent tissue from inadvertently migrating proximally to a point wherein the tissue could be inadvertently captured or clamped between the protrusions 74230 and their corresponding cavities 74136 in the staple cartridge 74120 as the anvil 74210 is closed.

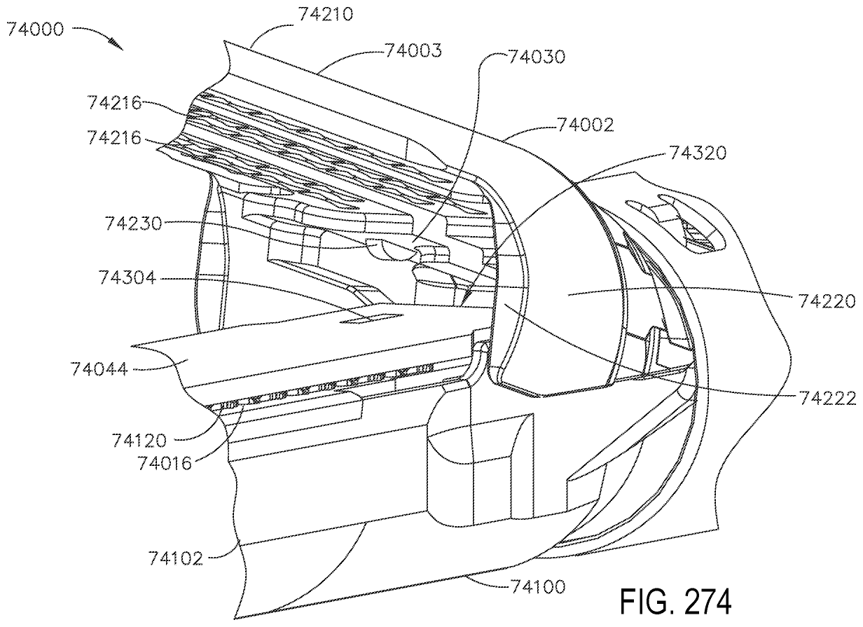

FIG. 274 illustrates use of the end effector 74000 with an adjunct 74300. The adjunct 74300 may comprise any of the adjuncts disclosed herein. The adjunct 74300 may comprise a single layer of any of the adjunct materials disclosed herein, woven or non-woven. In one application, the adjunct 74300 is positioned on the deck surface 74126 of the staple cartridge 74120 such that a proximal end 74302 of the adjunct extends proximally beyond the cavities 74136 in the proximal end 74124 of the staple cartridge 74120. Thereafter, the end effector 74000 may be manipulated to position the target tissue between the staple-forming surface 74216 of the anvil 74210 and the adjunct 74300. Once the target tissue has been properly positioned, the anvil 74210 is closed to clamp the target tissue between the anvil and the adjunct 74300. As the anvil is closed, the protrusions 74230 clamp portions of the adjunct 74300 in their respective cavities 74136 to retain the adjunct 74300 in position and prevent any movement of the adjunct 74300 (laterally or longitudinally) when the knife of the sled driver or firing actuator initially contacts and cuts through the adjunct 74300. Alternative embodiments of the adjunct 74300 additionally comprise cavities, detents, apertures, or holes 74304 that correspond to each protrusion 74230 and are adapted to receive the protrusion 74230 therein when the anvil 74210 is closed to thereby retain and prevent the adjunct 4300 from moving during the cutting and firing procedure.

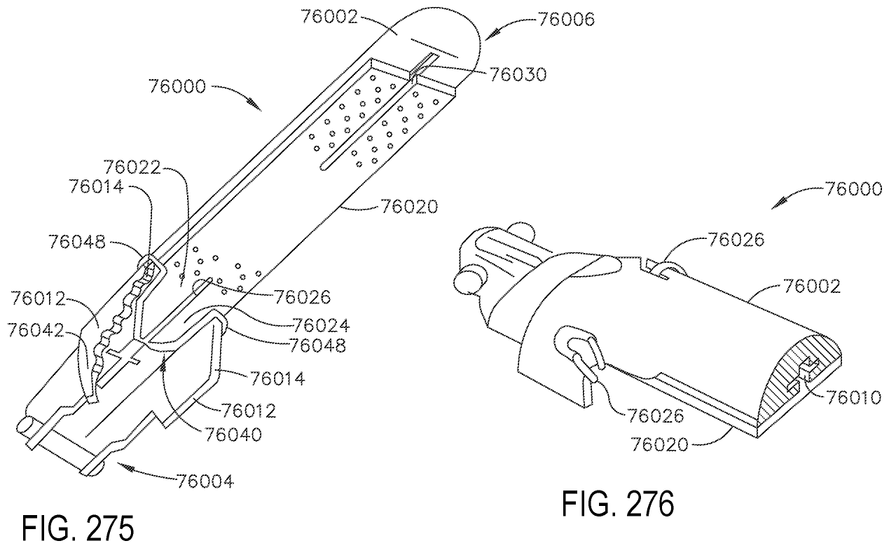

FIGS. 275 and 276 illustrate an anvil 76000 that may be advantageously employed with any of the surgical stapler end effectors disclosed herein. As will be discussed in further detail below, the anvil 76000 is configured to be used with an adjunct 76026. As can be seen in FIGS. 275 and 276, the anvil 76000 comprises an anvil body 76002 that comprises a proximal end portion 76004 and a distal end portion 76006. The anvil body 76002 further comprises a staple-forming undersurface 76008 that extends from the proximal end portion 76004 to the distal end portion 76006 and defines an elongated slot 76010 that is configured to accommodate passage of a firing actuator or sled driver of the surgical stapler. A tissue stop 76012 is formed on each side of the proximal end portion 76004 of the anvil 76000.

The adjunct 76020 may comprise any of the adjuncts disclosed herein with the additional features or attributes discussed below. As shown in FIG. 275, the adjunct 76020 comprises a proximal end 76022 that is configured to extend proximally past the distal edges 76014 of the tissue stops 76012. In the illustrated arrangement, the proximal end 76022 comprises a tapered end portion 76024. Also in the illustrated arrangement, the adjunct 76020 further comprises a proximal slot segment 76026 that extends partially into the adjunct 76020 and coincides with the longitudinal anvil slot 76010. Additionally in the illustrated arrangement, the adjunct 76020 further comprises a distal slot segment 76030 that extends partially inward from a distal end 76028 of the adjunct 76020 and also coincides with the anvil slot 76010. It will be appreciated that the proximal slot segment 76026 and the distal slot segment 76030 facilitates passage of the sled driver or firing actuator of the surgical stapler without contacting the adjunct 76020 during the lengths of the respective slot segments which could otherwise cause undesirable movement of the adjunct 76020 during the cutting and firing process. Other adjunct embodiments are contemplated that lack one or both of the proximal slot segment 76026 and the distal slot segment 86030.

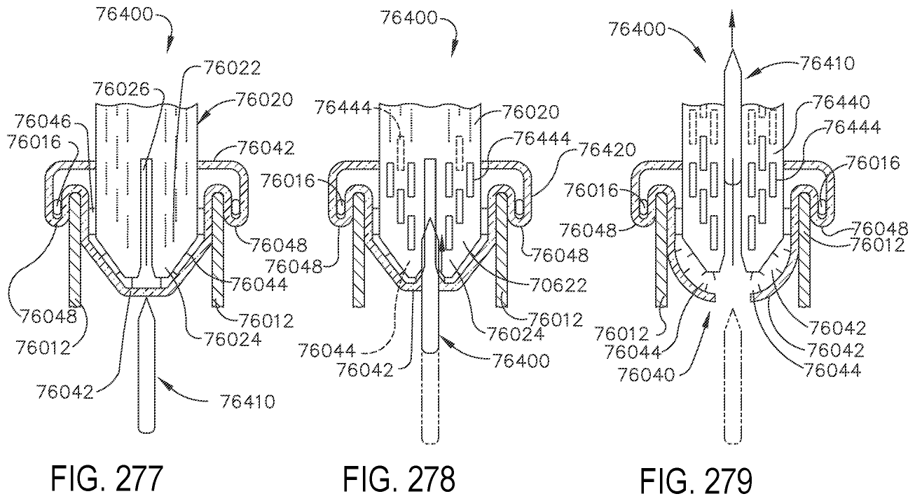

The adjunct 76020 further comprises an adjunct attachment assembly 76040 that is attached to or otherwise formed on the proximal end 76022 of the adjunct 76020 and is configured to releasably engage the proximal end portion 76004 of the anvil 76000. FIGS. 277, 278, and 279 illustrate portions of a surgical stapler end effector 76400 that comprises the anvil 76000 and the adjunct 76020. As shown in FIGS. 277-279, the adjunct attachment assembly 76040 comprises a severable attachment loop 76042 that is releasably attached to the proximal end 76022 of the adjunct 76020. The severable attachment loop 76042 is fabricated from the same material that comprises the adjunct 76020. However, the several attachment loop 76042 may be fabricated from one of the adjunct materials disclosed herein that differs from the adjunct material comprising the adjunct 76020.

As can be seen in FIGS. 275, 277, 278, and 279, the severable attachment loop 76042 is attached to the proximal end portion 76022 by a plurality of tearable links 76044 so that the severable loop 76042 spans across the tapered end portion 76024 and the proximal slot segment 76026 therein. The links 76044 may be formed by forming holes or apertures 76046 in the adjunct material between the links 76044. Additionally, the severable attachment loop comprises a first and second hook or loop portions 76048 formed therein that are configured to hookingly engage a corresponding slotted portion, protrusion, detent, hook or retention feature 76016 formed on each tissue stop 76012 or other portions of the proximal end portion 76004 of the anvil 76000.

FIG. 277 illustrates the position of the adjunct 76020 prior to commencing the cutting and firing process. As can be seen in FIG. 277, the knife 76410 of a sled driver or firing actuator of the surgical stapler is positioned proximal to the severable attachment loop 76042 on the adjunct 76020. FIG. 278 illustrates the position of the proximal end portion 76022 of the adjunct 76020 relative to the proximal-most staple cavities 76444 in a staple cartridge 76440 supported in the end effector 76400. As can be seen in FIG. 278, the proximal end portion 76022 and, more particularly, at least a portion of the tapered end portion 76024 of the adjunct 76020 is proximal to the proximal-most staple cavities 76444.

FIG. 278 illustrates commencement of the cutting and firing process. As can be seen in FIG. 278, the knife has severed the severable attachment loop 76042 as the knife 76410 is advanced distally through the end effector 76400. FIG. 279 illustrates the further advancement of the knife 76410 distally and some of the links 76042 being broken. Once the firing and cutting process has been completed and the adjunct 76020 has been severed and stapled to the severed target tissue that was clamped in the end effector 76400, the end effector jaws can be opened to release the target tissue and adjunct 76020 from the end effector. As the clinician removes the end effector from the stapled target tissue and adjunct, the remaining tearable links 76042 will break to release the adjunct attachment assembly 76040 from the adjunct 76020. Thus, the adjunct attachment assembly 76040 will be withdrawn from the patient with the end effector. Other embodiments are contemplated wherein severable hooks are formed in the adjunct material and are configured to otherwise releasably engage the hooks or protrusions on the anvil 76000. Such hooks would get torn or severed when the end effector is withdrawn from the stapled target tissue and adjunct to release the adjunct from the anvil leaving the remnants of the adjunct hooks with the staple pieces of adjunct to absorbed in the patient's body. In either cases, those of ordinary skill in the art will appreciate that the adjunct attachment assembly and hook arrangements serve to prevent the adjunct material from being longitudinally and laterally displaced as the knife pass through the adjunct material.

In addition to any of the adjunct retention arrangements and configurations disclosed herein, a temporary form of pressure sensitive adhesive 75000 may be applied to the device-contacting surface of the adjunct. Pressure sensitive adhesives may be characterized by one of more of the following properties: (1) aggressive and permanent tack; (2) adherence with no more than finger pressure; (3) sufficient ability to hold onto an adhered; and (4) sufficient cohesive strength to be removed cleanly from the adhered. These temporary pressure sensitive adhesives may be advantageously applied to certain portions of each adjunct to further enhance the adhesion of the adjunct to the anvil or staple cartridge to thereby prevent the adjunct from slipping, moving or bunching during firing. For example, these pressure sensitive adhesives may be applied only to a proximal portion of the device-contacting surface and/or they may be applied along the length of the adjuncts as well. Examples of such adhesives 75000 include, but are not limited to: poly vinyl pyrrolidone (PVP), poly vinyl acetate (PVA), acrylic based adhesives, and cyanoacrylate adhesives, among others. In one arrangement, a more aggressive (stickier) form of adhesive 75000 is applied only to the device-contacting surface of the adjunct that protrudes proximally beyond the distal edges of the tissue stops. For example, a small amount of PVP based adhesive may be applied to the device-contacting surface that is adjacent to the proximal end of the adjunct (and proximal to the distal edges of the tissue stops) to further enhance the retention of the proximal end of the adjunct to the device (anvil or staple cartridge). The properties of the adhesive may be further tuned to increase the adhesive's resistance to moisture intrusion/softening by reducing the amount of plasticizer added to the adhesive. Some adhesives such as PVP, however, become lubricious when wet making the portions of the device to which it is applied difficult to dry between uses. Thus, in at least some applications, it may be desirable to avoid applying those adhesives to the portions of the anvil that are proximal to the tissue stop distal edges.

FIG. 263 illustrates an amount of temporary pressure-sensitive adhesive 75000 applied to a proximal end portion of the first device-contacting surface 70064 in an alternative embodiment. FIG. 265 illustrates an amount of temporary pressure-sensitive adhesive 75000 applied to a proximal end portion of the first device-contacting surface 70074′ in an alternative embodiment.

Surgical stapling devices are commonly used in a variety of different surgical procedures to staple or otherwise fasten incised tissue. Such devices include an end effector that comprises a pair of jaws that are movable between an open position and closed positions. An elongate shaft is coupled to the end effector that facilitates insertion of the end effector into the patient, oftentimes through a cannula of a trocar or other constricted opening. The shaft may be coupled to a handle, or housing, that facilitates manual actuation of the end effector or the handle may comprise a motor or motors for applying actuation motions to the end effector. In alternative arrangements, the shaft may operably interface with a robotic system which is configured to manipulate the shaft and apply actuation motions to the end effector.

The shaft facilitates the transfer of opening and closing motions to the pair of jaws. A surgical staple cartridge is mounted in one of the jaws that comprises a channel and the other jaw comprises an anvil that is movably supported relative to the channel. The surgical staple cartridge commonly comprises a cartridge body that defines a deck surface that is oriented to face an underside of the anvil. Lines of surgical staples are received on staple drivers that are movably mounted in corresponding staple cavities formed in the cartridge body that open through the deck surface.

The end effector further includes a sled driver, or firing member or firing actuator, that is formed with camming surfaces or is configured to cooperate with a sled that includes camming surfaces or ramps configured to drive the staple drivers upward out of the staple cavities. The sled driver may additionally be provided with other camming members or guide tabs configured to slidably engage the anvil and the channel to retain the anvil at a proper spacing relative to the staple cartridge during the firing process. This spacing between the underside or forming surface of the anvil and the deck of the staple cartridge is often referred to as the “tissue gap.” The sled driver in those end effectors that are designed to cut tissue as well as staple or fasten tissue is equipped with a knife, or tissue cutting blade or surface. The shaft accommodates a movable firing beam or other known arrangement configured to drive the sled driver distally through the staple cartridge and retract the sled driver after the cutting and stapling procedure is completed.

The cartridge body is formed with a longitudinal slot that is configured to accommodate travel of the sled driver through the cartridge. The staple cavities open through the deck surface and are arranged to form an orientation of offset longitudinal rows on each side of the longitudinal slot. The staple cavities movably store staples therein. In use, the end effector is positioned adjacent the tissue to be cut and stapled (“target tissue”) with the jaws in the open position to enable the target tissue to be positioned between the underside of the anvil and the cartridge deck. The anvil can be moved toward the channel and/or the channel can be moved toward the anvil to motivate the end effector into the closed position. Once the target tissue has been desirably positioned between the anvil and the staple cartridge, the end effector is moved to a fully-closed position thereby clamping the target tissue between the anvil and the cartridge. Thereafter, the firing beam, firing bar, or other actuator arrangement is actuated to advance the sled driver and the sled distally through the cartridge. As the sled moves distally, the camming surfaces thereon sequentially cam the staple drivers upward in the staple cavities causing the staples temporarily supported on the staple drivers to pass through the clamped tissue and into forming contact with the underside of the anvil. The knife or cutting blade of the sled driver lags behind the sled, ensuring that the lines of staples are formed before the clamped tissue is incised.

The operation of surgical instruments during a surgical procedure can be optimized by gathering data during the surgical procedure. Information, or data, can be gathered during the surgical procedure using sensors strategically placed on the surgical instrument. Sensors positioned on and/or near the end effector can provide valuable information to an operating system of the surgical instrument and/or the user of the surgical instrument that can be used to optimize the operation of the surgical instrument and/or the outcome of the surgical procedure, for example. Such sensors can be used to detect various information including, for example, the presence of tissue between jaws of the end effector, a thickness of such tissue, and/or the presence of a foreign object between the jaws of the end effector. An operating system of the surgical instrument can use the detected information to modify an instrument operating parameter, such as a speed of a firing stroke, for example.

Not only can such sensors be used to detect a presence of adjacent articles, such sensors can also be used to detect a status of the end effector. In certain instances, sensors can be used to determine if a staple has left a particular staple cavity during a staple firing stroke. Detecting the staple leaving its associated staple cavity allows the surgical instrument and/or the user of the surgical instrument to identify a current stage of the staple firing stroke and/or if a staple firing stroke resulted in a successful staple formation, for example. Additional benefits associated with surgical instrument sensors is described in greater detail herein.

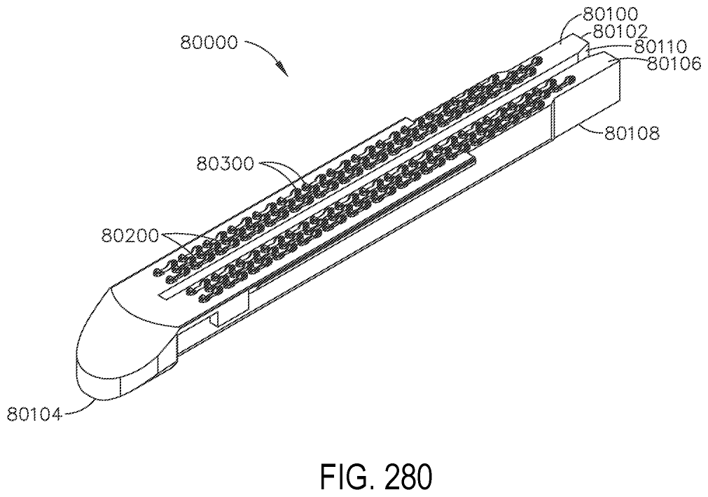

FIG. 280 depicts a staple cartridge 85000 for replaceable seating in a cartridge jaw of an end effector. The staple cartridge 85000 comprises a deck surface 85010 configured to oppose an anvil of the end effector when the staple cartridge 85000 is seated in the cartridge jaw. Staple cavities 85100, 85200, 85300 are defined in the staple cartridge 85000 and comprise staples movably stored therein. The staple cavities 85100, 85200, 85300 are arranged in six longitudinal rows, with three longitudinal rows on each side of a longitudinal slot 85005. The longitudinal rows of staple cavities extend between a proximal end 85002 and a distal end 85004 of the staple cartridge 85000. A first longitudinal row of staple cavities extends alongside a second longitudinal row of staple cavities on a first side of the longitudinal slot 85005. A third longitudinal row of staple cavities extends alongside the second longitudinal row of staple cavities on the first side of the longitudinal slot 85005. Staple cavities 85100 in the first longitudinal row are longitudinally-aligned with staple cavities 85300 in the third longitudinal row, while staple cavities 85200 from the second longitudinal row are longitudinally-offset from the staple cavities 85100, 85300 in the first and third longitudinal rows, respectively. While FIG. 280 depicts six longitudinal rows of staple cavities defined in the staple cartridge 85000, any suitable number of staple cavity rows and/or staple cavities is envisioned and can be selected based on a particular surgical procedure, for example.

The longitudinal slot 85005 extends between the proximal end 85002 and the distal end 85004 of the staple cartridge 85000 and is sized to receive a sled driver, or firing actuator, to eject staples out of the staple cartridge 85000 during a staple firing stroke. Various aspects of staple cartridges are described in greater detail in U.S. Pat. No. 9,844,369, the disclosure of which is herein incorporated by reference in its entirety.

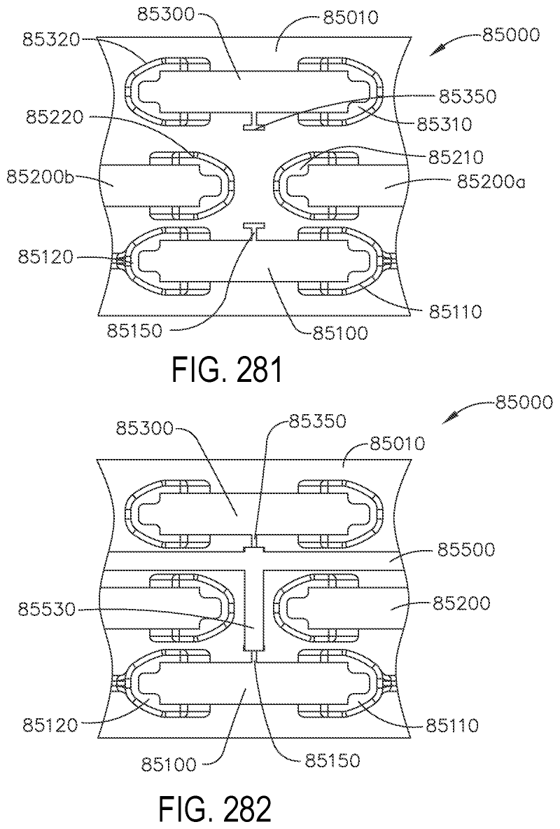

Referring now to FIG. 281, the staple cartridge 85000 further comprises projections extending from the deck surface 85010. The projections are integrated, or integrally-formed, with the staple cartridge 85000 and extend away from the deck surface 85010. Multiple, discrete projections surround at least a portion of the staple cavities defined in the staple cartridge 85000. A first projection 85110 surrounds a proximal portion of a staple cavity 85100 in the first longitudinal row of staple cavities. A second projection 85120 surrounds a distal portion of the staple cavity 85100 from the first longitudinal row. A third projection 85210 surrounds a distal portion of a first staple cavity 85200a from the second longitudinal row of staple cavities, and a fourth projection 85220 surrounds a proximal portion of a second staple cavity 85200b from the second longitudinal row. A fifth projection 85310 surrounds a proximal portion of a staple cavity 85300 from the third longitudinal row of staple cavities, and a sixth projection 85320 surrounds a distal portion of the staple cavity 85300 from the third longitudinal row.

One or more of the projections of the staple cartridge 85000 extend a staple cavity above the deck surface 85010. For instance, the first projection 85110 and the second projection 85120 extend the staple cavity 85100 above the deck surface 85010. As the staple stored in the staple cavity 85100 is ejected, or fired, during a staple firing actuation, the legs of the staple are supported by the projections 85110 and 85120 when the staple legs emerge above the deck surface 85010. In such instances, the possibility of the staple legs becoming misaligned with the staple forming pockets defined in the anvil is reduced. Projections 85210, 85220, 85310, and 85320 also comprise staple cavity extenders.

The projections extending from the deck surface 85010 define a valley, or recess, therebetween at least due to the longitudinally-offset orientation of the staple cavities 85200 in the second longitudinal row with respect to the staple cavities 85100, 85300 in the first and third longitudinal rows. The valley defines a continuous pathway extending between the staple cavities 85100, 85200, 85300 and their associated projections.

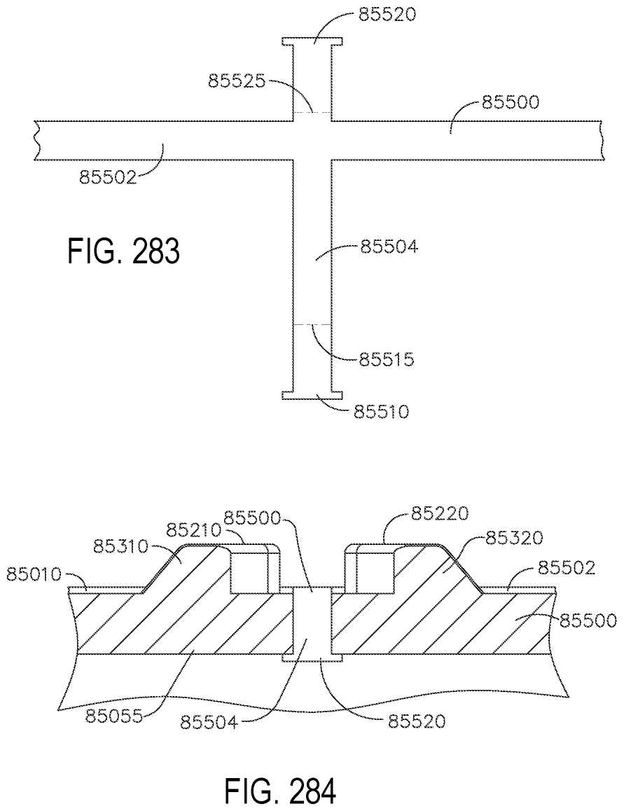

As discussed above, gathering and/or monitoring data during a surgical procedure is often desirable to optimize an operation and/or result of a surgical instrument. Such data can be gathered utilizing a sensor, or other suitable electrical traces capable of conducting signals. In certain instances, the sensor is positioned on the deck surface 85010 of the staple cartridge 85000 to detect data related to the staple cartridge 85000. Such data can include, for example, whether tissue is positioned adjacent the deck surface 85010 and/or a firing status of the staple cartridge 85000. A sensing array comprising one or more sensors can be integrated with a flexible substrate to form a flexible circuit 85500, as shown in FIG. 282 and shown coupled to the staple cartridge 85000 in FIG. 283. The valley defined amongst the staple cavities and projections on the deck surface 85010 provides an ideal, continuous pathway for a sensing array to extend.

In certain instances, the continuous pathway can be elevated, or stepped, with respect to the deck surface 85010. Stated another way, areas of the staple cartridge 85000 along the path of the flexible circuit 85500 extend above the primary deck surface 85010 to a first height. While such first height is greater than zero (i.e., the height of the primary deck surface 85010), it is less than or equal to a height to which the projections extend from the primary deck surface 85010. Such an elevated pathway allows for the sensing array to achieve a better contact with an adjacent tissue and/or adjunct material than if such a pathway was the same height of the primary deck surface 85010, for example.

The flexible circuit 85500 comprises a first portion 85502 configured to extend longitudinally along a deck surface of a staple cartridge. Stated another way, the first portion 85502 is configured to extend along the deck surface in between two longitudinal rows of staple cavities, such as in between the first longitudinal row and the second longitudinal row or in between the second longitudinal row and the third longitudinal row, for example. Such longitudinal extension of the flexible circuit 85500 allows for the positioning of sensors between longitudinal rows of staple cavities as well as allowing the flexible circuit 85500 to detect information regarding an adjacent article at various longitudinal positions of the staple cartridge 85000. In certain instances, the first portion 85502 is configured to extend along the deck surface in between the first longitudinal row of staple cavities and the longitudinal slot 85005. In certain instances, the first portion 85502 is configured to extend along the deck surface in between the third longitudinal row of staple cavities and an outer edge of the staple cartridge 85000.

The flexible circuit 85500 further comprises a second portion 85504 configured to extend laterally along the deck surface. Stated another way, the second portion 85504 is configured to extend along the deck surface in between a staple cavity 85100 from the first longitudinal row and a longitudinally-aligned staple cavity 85300 from the third longitudinal row. Ends 85510 of the second portion 85504 of the flexible circuit 85500 terminate in a T-shape; however, any suitable geometry is envisioned. Such lateral extension of the flexible circuit 85500 allows for the positioning of sensors in between adjacent staple pockets within the same longitudinal row.

Referring back to FIG. 281, a first T-shaped relief 85150 is defined in the staple cartridge 85000. Such first T-shaped relief 85150 extends into a staple cavity 85100 in the first longitudinal row of staple cavities. The first T-shaped relief 85150 is sized and shaped to receive a first end 85510 of the second portion 85504 of the flexible circuit 85500 to secure the flexible circuit 85500 in a desired orientation against the deck surface 85010. A second T-shaped relief 85350 is defined in the staple cartridge 85000 and extends into a staple cavity 85300 in the third longitudinal row of staple cavities. The second T-shaped relief 85350 is sized and shaped to receive a second end 85520 of the second portion 85504 of the flexible circuit 85500 to secure the flexible circuit 85500 in a desired orientation against the deck surface 85010. The first T-shaped relief 85150 is longitudinally-aligned with the second T-shaped relief 85350.

In attaching the flexible circuit 85500 to the staple cartridge 85000 as shown in FIGS. 283 and 284, a first end 85510 of the second portion 85504 of the flexible circuit 85500 is aligned with the first T-shaped relief 85150. In order to align the first end 85510 of the second portion 85504 with the first T-shaped relief 85150, the second end 85520 of the second portion 85504 extends in a direction away from the second T-shaped relief 85350. Once the first end 85510 of the second portion 85504 is depressed through the first T-shaped relief 85150, the flexible circuit 85500 is configured to bend and/or fold along a joint 85515. With the first end 85510 of the second portion 85504 depressed into the first T-shaped relief 85150 and the flexible circuit 85500 bent along joint 85515, the second end 85520 of the second portion 85504 extends away from the deck surface 85010 in the same direction as the projections. As the second end 85520 is pulled into alignment with the second T-shaped relief 85350, the first end 85510 engages a shelf 85055 defined in the staple cartridge 85000 and is secured in its desired position. Similarly, the second end 85520 is depressed through the second T-shaped relief 85350. Tension within the flexible circuit 85500 bends and/or folds the flexible circuit 85500 along a joint 85525 thereby engaging the second end 85520 with a shelf 85055 defined in the staple cartridge 85000. In certain instances, every staple cavity within the first longitudinal row and the third longitudinal row comprises such a T-shaped relief; however, any frequency of T-shaped reliefs suitable to maintain the flexible circuit 85500 against the deck surface 85010 in its desired position is envisioned. Stated another way, the T-shaped reliefs can be defined in every other staple cavity within the first and third longitudinal rows, for example.

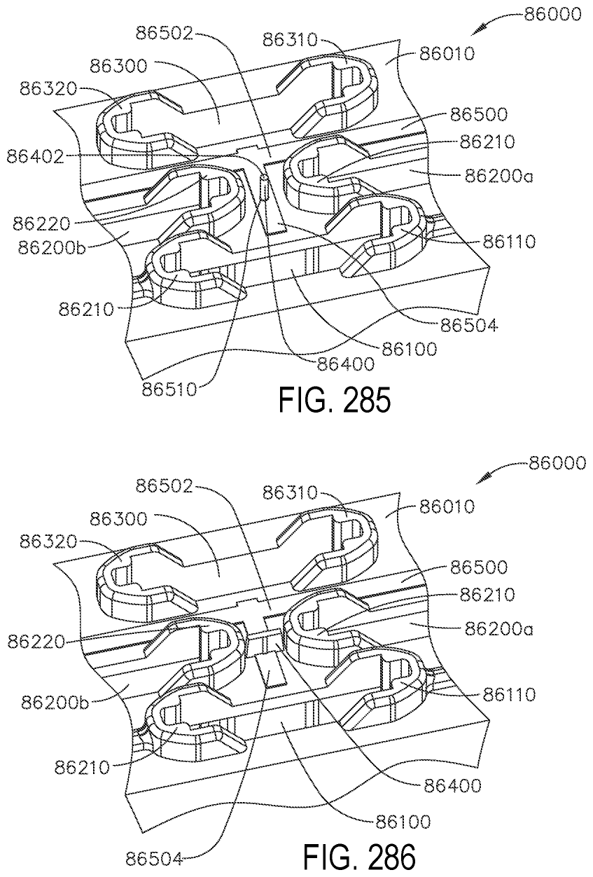

Turning now to FIGS. 285 and 286, a staple cartridge 86000 similar in many respects to staple cartridge 85000 is depicted. The staple cartridge 86000 further comprises projections extending from the deck surface 86010. The projections are integrated, or integrally-formed, with the staple cartridge 86000 and extend away from the deck surface 86010. Multiple, discrete projections surround at least a portion of the staple cavities defined in the staple cartridge 86000. A first projection 86110 surrounds a proximal portion of a staple cavity 86100 in a first longitudinal row of staple cavities. A second projection 86120 surrounds a distal portion of the staple cavity 86100 from the first longitudinal row. A third projection 86210 surrounds a distal portion of a first staple cavity 86200a from a second longitudinal row of staple cavities, and a fourth projection 86220 surrounds a proximal portion of a second staple cavity 86200b from the second longitudinal row. A fifth projection 86310 surrounds a proximal portion of a staple cavity 86300 from a third longitudinal row of staple cavities, and a sixth projection 86320 surrounds a distal portion of the staple cavity 86300 from the third longitudinal row.

The projections extending from the deck surface 86010 define a valley, or recess, therebetween at least due to the longitudinally-offset orientation of the staple cavities 86200 in the second longitudinal row with respect to the staple cavities 86100, 86300 in the first and third longitudinal rows. The valley defines a continuous pathway extending between the staple cavities 86100, 86200, 86300 and their associated projections.

A flexible circuit 86500, similar in many respects to flexible circuit 85500, is configured to be secured against a deck surface 86010 of the staple cartridge 86000. The flexible circuit 86500 comprises a first portion 86502 configured to extend longitudinally along a deck surface of a staple cartridge. Stated another way, the first portion 86502 is configured to extend along the deck surface in between two longitudinal rows of staple cavities, such as in between the first longitudinal row and the second longitudinal row or in between the second longitudinal row and the third longitudinal row, for example. In certain instances, the first portion 86502 is configured to extend along the deck surface in between the first longitudinal row of staple cavities and a longitudinal slot. In certain instances, the first portion 86502 is configured to extend along the deck surface in between the third longitudinal row of staple cavities and an outer edge of the staple cartridge 86000.

The flexible circuit 86500 further comprises a second portion 86504 configured to extend laterally along the deck surface 86010. Stated another way, the second portion 86504 is configured to extend along the deck surface in between a staple cavity 86100 from the first longitudinal row and a longitudinally-aligned staple cavity 86300 from the third longitudinal row.

A through hole 86510, or aperture, is defined in the flexible circuit 86500. The through hole 86510 is sized to receive a trace retention feature 86400 of the staple cartridge 86000 therethrough. As shown in FIG. 285, the trace retention feature 86400 comprises a post extending from the deck surface 86010 in a first, unmelted, configuration. In certain instances, the trace retention feature 86400 is integrally-formed with the staple cartridge 86000. While the through hole 86510 is shown as being defined in the second portion 86504 of the flexible circuit 86500, the through hole 86510 is also suitably defined in the first portion 86502 of the flexible circuit 86500, or wherever a corresponding trace retention feature is present.

The trace retention feature 86400 is comprised of a meltable material, such as a polymer, for example. In securing the flexible circuit 86500 against the deck surface 86010 of the staple cartridge 86000, the through hole 86510 of the flexible circuit 86500 is aligned with the trace retention feature 86400 of the staple cartridge 86000. Once desirably aligned, the trace retention feature 86400 is inserted through the through hole 86510 defined in the flexible circuit 86500. The post, or trace retention feature 86400 is then heated to a degree sufficient to melt an end 86402 of the trace retention feature 86400 into a second configuration as shown in FIG. 286. In its second configuration, the trace retention feature 86400 is no longer sized to easily and/or harmlessly move relative to the through hole 86510 defined in the flexible circuit 86500. In certain instances, a portion of the trace retention feature 86400 is melted over an entire width of the flexible circuit 86500 and onto the surrounding, underlying deck surface 86010. In any event, the flexible circuit 86500 is secured against the deck surface 86010 when the trace retention feature 86400 is in its second, melted, configuration.

In addition to securing the trace retention feature 86400 to the staple cartridge 86000, the trace retention feature 86400 provides a tissue-gripping function. Tissue, or an adjunct layer, positioned against the deck surface 86010 is maintained in position generally by the clamping force exerted thereon by jaws of an end effector in a closed configuration and more locally by the projections 86110, 86120, 86210, 86220, 86310, 86320 extending from a cartridge deck surface 86010. In instances where the staple cartridge 86000 comprises a post 86400 as a trace retention feature 86400, such post also interfaces with tissue, or an adjunct material, positioned there against. Melting the end 86402 of the post 86400 increases the overall surface area of the end 86402, and thus, increases the surface area of the interface between the post 86400 and adjacently-positioned tissue or adjunct material. An increased interface surface area allows for a desirably stronger gripping force to be exerted on the adjacent tissue or adjunct material.

As described with respect to the staple cartridge 85000 depicted in FIGS. 280-284, in certain instances, a continuous pathway defined by the flexible circuit 86500 can be elevated, or stepped, with respect to the deck surface 86010. Stated another way, areas of the staple cartridge 86000 along the path of the flexible circuit 86500 extend above the primary deck surface 86010 to a first height. While such first height is greater than zero (i.e., the height of the primary deck surface 86010), it is less than or equal to a height to which the projections extend from the primary deck surface 86010. Such an elevated pathway allows for the sensing array to achieve a better contact with an adjacent tissue and/or adjunct material than if such a pathway was the same height of the primary deck surface 86010, for example.

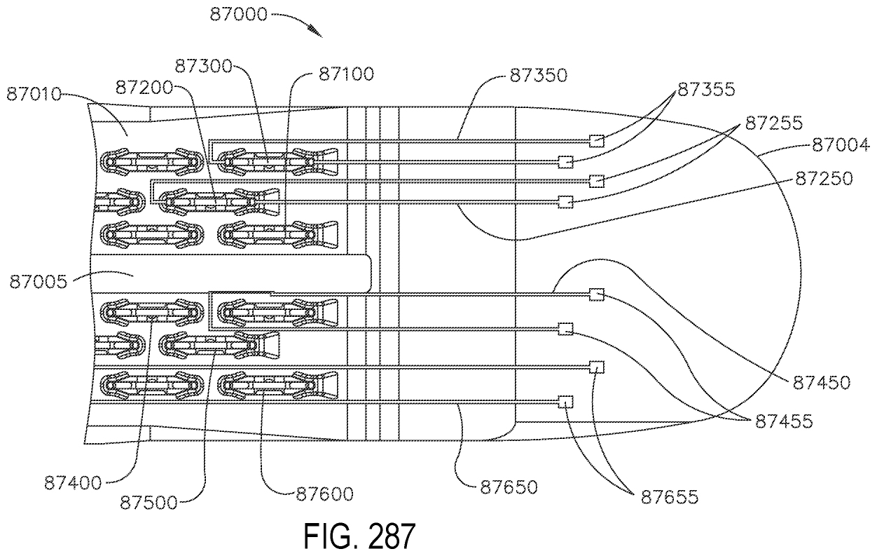

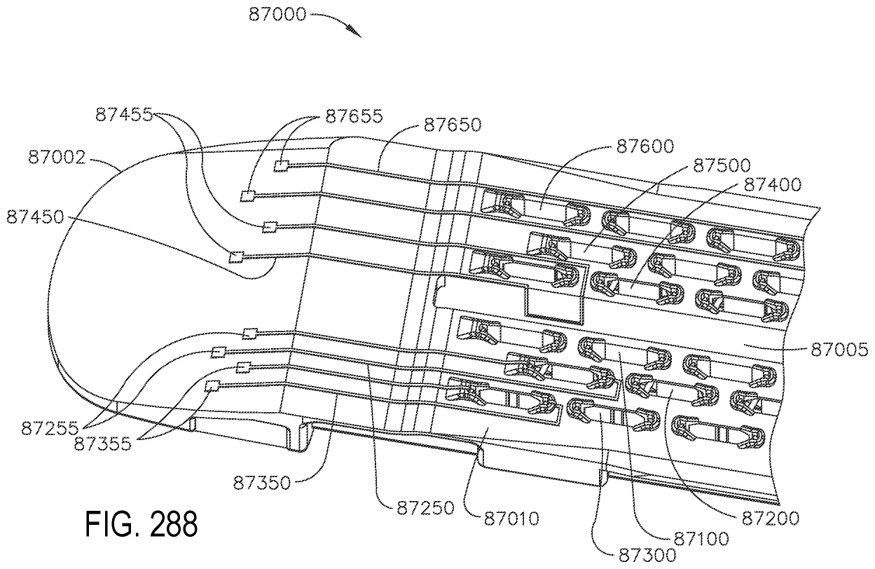

Referring now to FIGS. 287 and 288, a staple cartridge 87000 is depicted with electrical traces printed directly on the staple cartridge 87000. Such direct printing removes the need for a physical securement mechanism and allows for an unbounded sensing area. Staple cartridge 87000 is similar in many respects to staple cartridges 85000, 86000. The staple cartridge 87000 comprises a deck surface 87010 configured to oppose an anvil of the end effector when the staple cartridge 87000 is seated in a cartridge jaw. Staple cavities 87100, 87200, 87300, 87400, 87500, 87600 are defined in the staple cartridge 85000 and comprise staples movably stored therein. The staple cavities 87100, 87200, 87300, 87400, 87500, 87600 are arranged in six longitudinal rows, with three longitudinal rows on each side of a longitudinal slot 87005. The longitudinal rows of staple cavities extend between a proximal end and a distal end 87004 of the staple cartridge 87000. A first longitudinal row of staple cavities 87100 extends alongside a second longitudinal row of staple cavities 87200 on a first side of the longitudinal slot 87005. A third longitudinal row of staple cavities 87300 extends alongside the second longitudinal row of staple cavities 87300 on the first side of the longitudinal slot 87005. A fourth longitudinal row of staple cavities 87400 extends alongside a fifth longitudinal row of staple cavities 87500 on a second side of the longitudinal slot 87005. A sixth longitudinal row of staple cavities 87600 extends alongside the fifth longitudinal row of staple cavities 87500 on the second side of the longitudinal slot 87005. While six longitudinal rows of staple cavities are depicted as being defined in the staple cartridge 87000, any suitable number of staple cavity rows and/or staple cavities is envisioned and can be selected based on a particular surgical procedure, for example.

The longitudinal slot 87005 extends between the proximal end and the distal end 87004 of the staple cartridge 87000 and is sized to receive a sled driver, or firing actuator, to eject staples out of the staple cartridge 87000 during a staple firing stroke. Various aspects of staple cartridges are described in greater detail in U.S. Pat. No. 9,844,369, the disclosure of which is herein incorporated by reference in its entirety.

A first electrical trace 87250 is printed on the staple cartridge 87000 and wraps from a distal portion of the staple cartridge 87000 along the deck surface 87010 between the second longitudinal row of staple cavities 87200 and the third longitudinal row of staple cavities 87300. The first electrical trace 87250 continues directly into a distal-most staple cavity 87200 from the second longitudinal row and returns to the distal portion of the staple cartridge 87000. In addition to sensing a presence of tissue and/or an adjunct material against the deck surface 87010 in between longitudinal rows, extension of the first electrical trace 87250 into the staple cavity 87200 allows for the detection of a presence, or lack thereof, of a staple therein. In instances where a staple is still detected within the staple cavity 87200, the surgical instrument and/or the user of the surgical instrument is aware the staple firing stroke is incomplete. The first electrical trace 87250 comprises pads, or sensors, 87255 configured to detect a position of tissue and/or adjunct material along the deck surface 87010. Such feedback provides a user of the surgical instrument and/or the surgical instrument itself the ability to determine if repositioning of the surgical instrument is necessary, or desirable for an optimal outcome. While the pads 87255 are depicted as being at the terminal ends of the first electrical trace 87250, such pads 87255 can be positioned at any point and in any desired frequency along the first electrical trace 87250. While the first electrical trace 87250 is only depicted as extending into the distal-most staple cavity 87200 from the second longitudinal row of staple cavities, it is envisioned that the first electrical trace 87250 can extend into any suitable number of staple cavities from the second longitudinal row.

Similarly, a second electrical trace 87350 is printed on the staple cartridge 87000 and wraps from the distal portion of the staple cartridge 87000 along the deck surface 87010 between the third longitudinal row of staple cavities 87300 and an exterior edge of the staple cartridge 87000. The second electrical trace 87350 continues directly into a distal-most staple cavity 87300 from the third longitudinal row and returns to the distal portion of the staple cartridge 87000. The second electrical trace 87350 comprises pads, or sensors, 87355 configured to detect a position of tissue and/or adjunct material along the deck surface 87010. While the pads 87355 are depicted as being at the terminal ends of the second electrical trace 87350, such pads 87355 can be positioned at any point and in any desired frequency along the second electrical trace 87350. While the second electrical trace 87350 is only depicted as extending into the distal-most staple cavity 87300 from the third longitudinal row of staple cavities, it is envisioned that the second electrical trace 87350 can extend into any suitable number of staple cavities from the third longitudinal row.

A third electrical trace 87450 is printed on the staple cartridge 87000 and wraps from the distal portion of the staple cartridge 87000 along the deck surface 87010 between the fourth longitudinal row of staple cavities 87400 and the fifth longitudinal row of staple cavities 87500. Instead of returning to the distal portion of the staple cartridge 87000 through a staple cavity of the fourth row, the third electrical trace 87450 wraps back to the distal portion of the staple cartridge 87000 through the longitudinal slot 87005. In addition to sensing a presence of tissue and/or an adjunct material against the deck surface 87010 in between longitudinal rows, extension of the third electrical trace 87450 into the longitudinal slot 87005 allows for the detection of a firing actuator. The third electrical trace 87450 can extend into the longitudinal slot 87005 at a particular location so as to indicate a completion of the staple firing stroke when the firing actuator is detected at the particular location. The third electrical trace 87450 can extend into the longitudinal slot 87005 at an alternative and/or additional location(s) to detect the position of the firing actuator at any desired instant during the staple firing stroke. The third electrical trace 87450 comprises pads, or sensors, 87455 configured to detect a position of tissue and/or adjunct material along the deck surface 87010. While the pads 87455 are depicted as being at the terminal ends of the third electrical trace 87450, such pads 87455 can be positioned at any point and in any desired frequency along the third electrical trace 87450.

Similarly, a fourth electrical trace 87650 is printed on the staple cartridge 87000 and wraps from the distal portion of the staple cartridge 87000 along the deck surface 87010 between the sixth longitudinal row of staple cavities 87600 and an exterior edge of the staple cartridge 87000. The fourth electrical trace 87650 extends the entire length of the sixth longitudinal row of staple cavities 87600 and ultimately returns to the distal portion of the staple cartridge 87000 along the deck surface 87010 between the sixth longitudinal row of staple cavities 87600 and the fifth longitudinal row of staple cavities 87500. The fourth electrical trace 87650 comprises pads, or sensors, 87655 configured to detect a position of tissue and/or adjunct material along the deck surface 87010. While the pads 87655 are depicted as being at the terminal ends of the fourth electrical trace 87650, such pads 87655 can be positioned at any point and in any desired frequency along the fourth electrical trace 87650. For example, such pads 87655 can be placed every 10 mm along the length of the staple cartridge 87000.

While FIGS. 287 and 288 depict a single cartridge comprising four different electrical traces, it is envisioned that any combination of the depicted electrical traces can be used in any suitable longitudinal row and/or on any single staple cartridge.

A cartridge jaw, or channel, of a surgical instrument end effector is sized to receive a cartridge of a particular size. However, it is oftentimes desirable to use different staple types, or sizes, during a particular surgical procedure. Stated another way, different staples are preferable for use with different tissues and/or for different tissue sealing outcomes. In an effort to reduce the surgical instruments used during the surgical procedure, it is desirable to have a universally-sized staple cartridge that fits in a single cartridge jaw that is capable of storing staples of different sizes and/or geometries. Differences in staples include, for example, composition material, staple leg diameter, and/or staple length, for example.

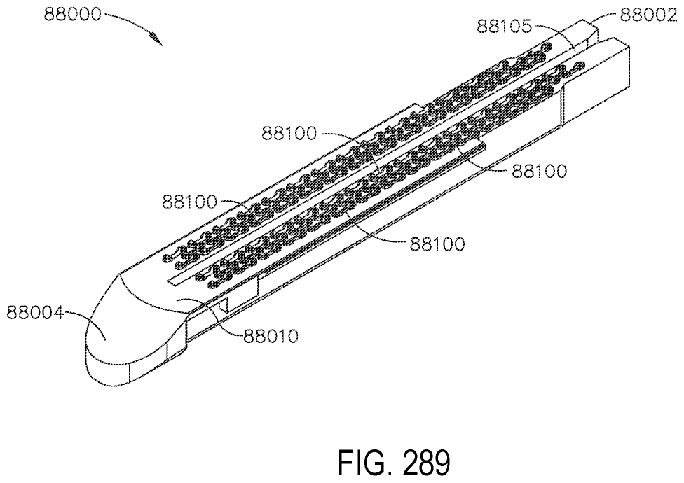

FIG. 289 depicts a staple cartridge 88000 that is seatable in a cartridge jaw of an end effector. In use, the staple cartridge 88000 can be readily seated into the cartridge jaw during a surgical procedure, fired, and then removed from the cartridge jaw so that the now-spent staple cartridge 88000 can be replaced with another staple cartridge. That said, embodiments are envisioned in which the staple cartridge 88000 is not readily removable from the cartridge jaw and is not replaceable during a surgical procedure. The staple cartridge 88000 comprises a cartridge body including a deck surface 88010 configured to oppose an anvil of the end effector when the staple cartridge 88000 is seated in the cartridge jaw and the end effector is moved into a closed configuration. Staple cavities 88100 are defined in the cartridge body of the staple cartridge 88000 that are each configured to receive and store a staple therein. The staple cavities 88100 are arranged in six longitudinal rows, with three longitudinal rows on each side of a longitudinal slot 88005 defined in the cartridge body. The longitudinal rows of staple cavities extend between a proximal end 88002 and a distal end 88004 of the staple cartridge 88000. In alternative embodiments, any suitable number of staple cavity rows and/or staple cavities is envisioned and can be selected based on the needs of a particular surgical procedure, for example.

The longitudinal slot 88005 extends between the proximal end 88002 and the distal end 88004 of the staple cartridge 88000 and is sized to receive a firing driver, or firing actuator, to eject staples out of the staple cartridge 88000 during a staple firing stroke. Various aspects of staple cartridges are described in greater detail in U.S. Pat. No. 9,844,369. The entire disclosure of U.S. Pat. No. 9,844,369, entitled SURGICAL END EFFECTORS WITH FIRING ELEMENT MONITORING ARRANGEMENTS, which issued on Dec. 19, 2017, is incorporated by reference herein.

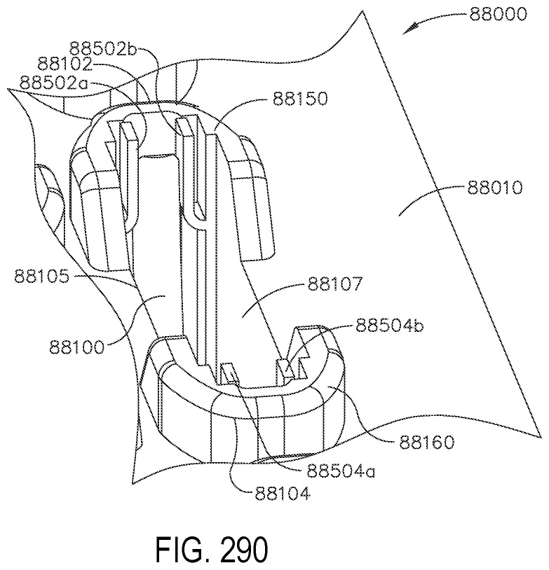

Referring now to FIG. 290, an enlarged view of a staple cavity 88100 defined in the staple cartridge 88000 is depicted. The staple cavity 88100 shown in FIG. 290 is representative of all staple cavities defined in the staple cartridge 88000. In certain instances, all of the staple cavities defined in the staple cartridge can comprise the same features, whereas in other instances, only a select number of staple cavities comprise the same features. For example, features of the staple cavities can vary laterally amongst the longitudinal rows or can vary longitudinally across the staple cartridge 88000.

The staple cavity 88100 comprises a proximal end 88102 and a distal end 88104. A first lateral wall 88105 and a second lateral wall 88107 span between the proximal and distal ends 88102, 88104. The first lateral wall 88105 extends along a first side of the staple cavity 88100, whereas the second lateral wall 88107 extends along a second side of the staple cavity 88100 opposite of the first side.

A staple is movably stored in the staple cavity 88100. A first pocket extender, or projection, 88150 extends a first distance from the deck surface 88010 of the staple cartridge 80000, and a second pocket extender, or projection 88160 extends a second distance from the deck surface 88010 of the staple cartridge 80000. The first distance of the first pocket extender 88150 is the same as the second distance of the second pocket extender 88160; however, other embodiments are envisioned in which the first distance and the second distance are not the same. The first projection 88150 surrounds at least a portion of the proximal end 88102 of the staple cavity 88100, and the second projection 88160 surrounds at least a portion of the distal end 88104 of the staple cavity 88100. While the first projection 88150 and the second projection 88160 are shown in FIG. 290 as being discrete, or independent, from one another, the first projection 88150 and the second projection 88160 can be coupled, or otherwise connected to one another.

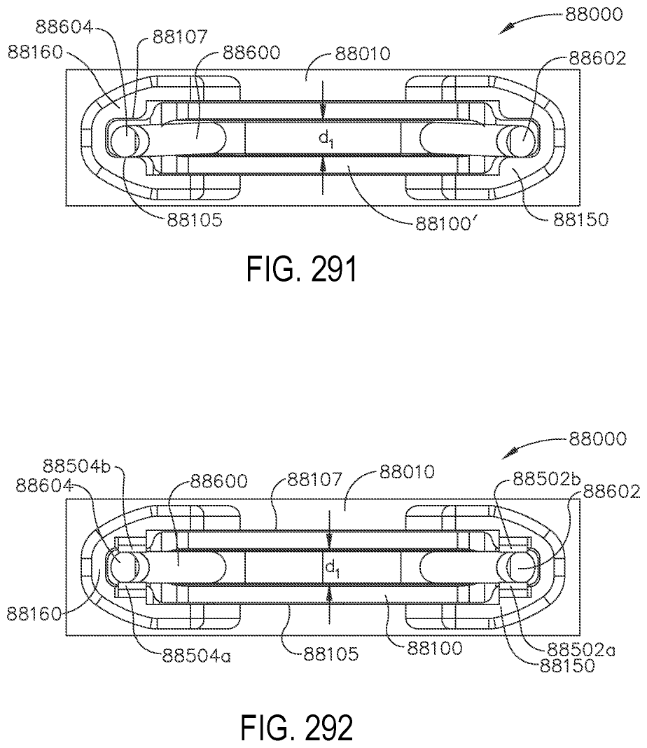

As discussed above, each staple cavity 88100 is configured to store a staple therein. Referring to FIG. 291, a staple 88600 is positioned in the staple cavity 88100. The staple 88600 is comprised of a metal wire, such as stainless steel, for example, and comprises a base, a first leg 88602 extending from a first end of the base, and a second leg 88604 extending from a second, or opposite, end of the base. During the staple firing stroke, a staple driver of the staple cartridge 88000 is pushed upwardly within the staple cavity 88100 which contacts the base of the staple 88600 and pushes the staple 88600 upwardly from an unfired position to a fired position. In such instances, the tips of the staple legs 88602, 88604 come into contact with an anvil positioned opposite the staple cartridge 88000 and the staple 88600 is deformed from an unfired configuration, such as a V-shaped configuration, for example, into a fired configuration, such as a B-shaped configuration, for example. The staple 88600 can comprise any suitable height when it is in its unfired configuration. For instance, the staple 88600 can comprise an unfired height of approximately 3.0 mm, approximately 4.0 mm, or approximately 5.0 mm, for example. Depending on the height of the staple 88600, the tips of the staple 88600 may be recessed with respect to, flush with, or extend above the deck surface 88010 when the staple 88600 is in its unfired position. In any one of these arrangements, the first and second projections 88150 and 88160 can prevent, or at least reduce the possibility of, the staple tips from snagging on patient tissue and/or being damaged or disturbed within the staple cavity 88100. Moreover, the first and second projections 88150, 88160 provide additional support, or guidance, to the staple 88600 to maintain the staple 88600 in a desired alignment, or orientation, as the staple 88600 is lifted out of the staple cartridge 88000 toward the opposing anvil during the staple firing stroke.

As described herein, it is desirable for a cartridge body of a staple cartridge to be compatible with, or able to be used with, staples of varying sizes and/or geometries. For instance, as described above, staples can have different unformed heights and, in various embodiments, it is desirable for the staple cavities of a cartridge body to be able to receive short staples or tall staples. Also, for instance, wire staples can have different diameters and, similar to the above, it is desirable for the staple cavities of a cartridge body to be able to receive staples having a thick diameter or a thin diameter. To achieve this, the staple cavities of a staple cartridge must be configured appropriately to receive a variety of staples therein. Stated another way, the staple cavities must be large enough to accommodate receiving a thick wire staple, while also being able to receive a thin wire staple, for example. More specifically, as shown in FIGS. 291-294, the staple cartridge 88000 can be used with staples having different diameters. The staple cartridge 88000 can be used with first staples 88600 having a first diameter d₁ of 0.0096 inches, for example. The staple cartridge 88000 can also be used with second staples 88600′ having a second diameter d₂ of 0.0079 inches, for example. The second diameter d₂ is less than the first diameter d₁.

Regardless of the size and/or shape of the staple movably stored in the staple cavity, it is desirable, in various embodiments, for the staple to be centered within the staple cavity for optimal staple firing and/or formation. In various embodiments, it is desirable for the staple to be positioned equidistant from the first lateral wall 88105 and the second lateral wall 88107 of the staple cavity 88100 and in an upright orientation. The reader should appreciate that the first and second lateral sidewalls 88105 and 88107 may be not be perfectly straight or planar owing to manufacturing variations, for instance. As such, the center of a staple cavity 88100 may be approximated by an average midpoint measured laterally between the first and second lateral sidewalls 88105 and 88107 along the longitudinal length of the staple cavity 88100. Moreover, along these lines, the staple positioned in the staple cavity 88100 need not be perfectly centered between the first and second lateral sidewalls 88105 and 88107 in order for the staple to be equidistant between the first and second lateral sidewalls 88105 and 88107. For instance, the longitudinal center plane of a staple can be shifted laterally—in either lateral direction—up to 25% of the lateral width of the staple relative to the longitudinal center plane of the staple cavity 88100, for example, for the staple to be positioned equidistant between the first and second lateral sidewalls 88105 and 88107. Similarly, the reader should appreciate that a staple may also not be straight or planar owing to manufacturing variations. As such, similar to the above, the longitudinal center plane of a staple may be approximated by an average midpoint measured laterally between the first and second lateral sides of the staple. A staple can be sufficiently equidistant between the first lateral wall 88105 and the second lateral wall 88107 if the staple tips are properly aligned with, or registered with, the forming pockets defined in the anvil positioned opposite the staple cartridge 88000.

As shown in FIG. 294, placement of the second staples 88600′ having a smaller diameter into the universal staple cavities 88100′ of a staple cartridge 88000′, without additional structure, results in the second staples 88600′ being haphazardly stored in the staple cavities 88100′. Such loose, non-uniform storage of second staples can result in the creation of a non-uniform staple line, for example. In various instances, the second staples 88600′ may not remain centered in the universal staple cavities 88100′ and, as such, the second staples 88600′ may have unintended angular offset when ejected during the staple firing stroke. Such angular offset can result in the second staples failing to strike the appropriate forming pockets defined in the opposing anvil, leading to increased staple malformations. In some instances, placement of the first staples 88600 having a larger diameter into the universal staple cavities 88100′ of the staple cartridge 88000′, without additional structure, can also lead to the first staples 88600 being off-center, as shown in FIG. 292. For example, first and second legs 88604, 88604 of the first staple 88600 can be tilted toward and/or rested against the first lateral wall 88105 of the staple cavity 88100′. In some instances, the legs of an uncentered staple can gouge into the sidewalls of a staple cavity which can prevent the staple from being ejected from the staple cavity.

In an effort to ensure the proper alignment of a staple within a universal staple cavity 88100, the staple cavity 88100 comprises one or more staple alignment features 88502, 88504 integrally formed with the body of the staple cartridge 88000. As shown in FIG. 290, the staple alignment features 88502, 88504 are integrally-formed with the first lateral wall 88105 and the second lateral wall 88107 of the staple cavity 88100. The staple alignment features 88502, 88504 comprise spring tabs, or arms, but can comprise any suitable configuration. A first pair of spring tabs 88502a, 88502b extend into the staple cavity 88100 from the first lateral wall 88105 and the second lateral wall 88107, respectively, at a proximal portion of the staple cavity 88100. A second pair of spring tabs 88504a, 88504b extend into the staple cavity 88100 from the first lateral wall 88105 and the second lateral wall 88107, respectively, at a distal portion of the staple cavity 88100. The spring tabs 88502, 88504 are configured to bias against, or otherwise contact, first and second legs 88602, 88604 of the staple, respectively, while the staple is movably stored in the staple cavity 88100. Such engagement between the spring tabs 88502, 88504 and the staple legs 88602, 88604 serves to maintain the staple 88600, for instance, in a centered orientation within the staple cavity 88100.

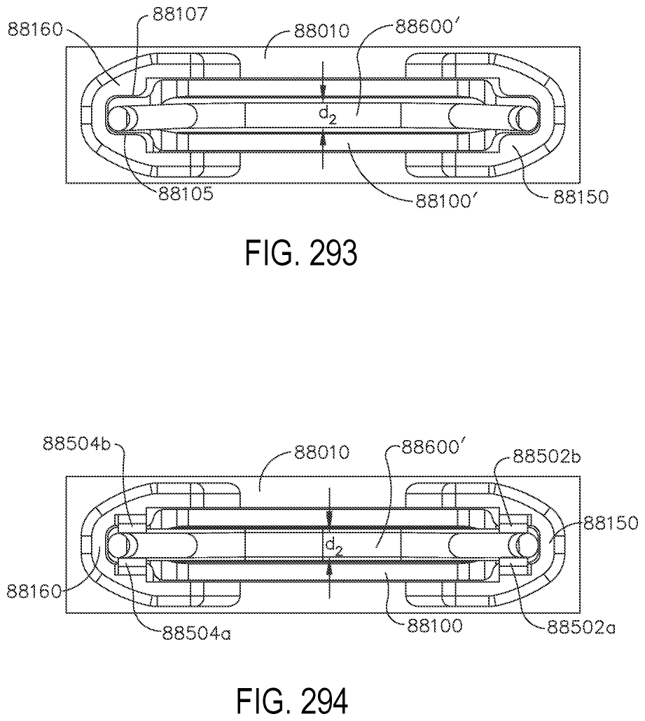

As shown in FIGS. 291 and 293, the spring tabs 88502, 88504 are configured to maintain the staple in a centered, upright orientation within the staple cavity 88100 regardless of the staple's diameter and/or geometry. Due to their resilient nature, the spring tabs 88502, 88504 are configured to be biased toward the first lateral wall 88105 and the second lateral wall 88107 to different degrees based at least in part on the diameter of the staple movably stored therein. FIG. 290 depicts the spring tabs 88502, 88504 in their unbiased, or natural, configuration as no staple is present in the staple cavity 88100. A distance spanning between the pair of spring tabs 88502a, 88502b is referred to herein as a staple gap width. In the current embodiment, the smallest staple diameter compatible for use with the staple cartridge 88000 is a diameter equal to the staple gap width when the spring tabs 88502a, 88502b are in their unbiased configuration. Conversely, the largest staple diameter compatible for use with the staple cartridge 88000 is a diameter equal to the staple gap width when the spring tabs 88502a, 88502b are in their fully-biased configuration, i.e., the spring tabs 88502a, 88502b biased against the first lateral wall 88105 and the second lateral wall 88107, respectively.

As shown in FIG. 293, the staple cavity 88100 movably stores the second staple 88600′ having a diameter d₂ smaller than the diameter d₁ of the first staple 88600 shown stored in the staple cavity 88100 in FIG. 291. In such instances, the spring tabs 88502, 88504 remain closer to their unbiased configuration to make sufficient contact with the staple 88600′ to maintain the staple 88600′ in a centered, upright orientation. In instances where the staple comprises a larger diameter, as shown in FIG. 291, the spring tabs 88502, 88504 are biased closer to the first lateral wall 88105 and the second lateral wall 88107, respectively, to accommodate for the larger diameter d₁ of the first staple 88600.

While the staple alignment features 88502, 88504 are depicted as arms in the form of spring tabs, any suitable resilient member, such as a spring, a layer of foam, and/or other resilient material, for example, is envisioned. Furthermore, the staple alignment features 88502, 88504 extend a depth into the staple cavities sufficient to support staples having varying staple leg lengths that are suitable, or otherwise desirable, for use with the staple cartridge 88000. While the staple alignment features 88502, 88504 are depicted as being positioned on both lateral side walls of the staple cavity and at both proximal and distal ends of the staple cavity, any position and/or quantity of staple alignment features are envisioned that are suitable to support, or otherwise accommodate staples of different diameters.

As described above, the staple alignment features 88502, 88504 are in contact with a staple positioned in the staple cavity 88100 to position the staple within the staple cavity 88100. As also described above, each alignment feature 88502, 88504 is configured to apply a force to the staple. Such forces can act normally or perpendicularly to surface of the staple and, when the staple is ejected from the staple cavity 88100 during the staple firing stroke, friction forces between the alignment features 88502, 88504 resist, but do not prevent, the ejection of the staple from the staple cavity 88100. The magnitude of the friction forces are proportional to the magnitude of the forces applied to the staple by the alignment features 88502, 88504. Such an arrangement advantageously retains, i.e., releasably retains, the staple in the staple cavity 88100 and prevents, or at least reduces the possibility of, the staple from accidentally falling out of the staple cavity 88100. In various embodiments, the alignment features 88502, 88504 hold the staple in position without the staple touching any other part of the staple cavity 88100. In other embodiments, the staple is in contact with one or more walls of the staple cavity 88100 in addition to being in contact with the alignment features 88402, 88504. In at least one such embodiment, the staple is substantially V-shaped and the legs of the staple are in contact with the proximal and distal end walls of the staple cavity 88100.

As discussed above, one or more of the projections of the staple cartridge 88000 extend a staple cavity above the deck surface 88010. For instance, the first projection 88150 and the second projection 88160 extend a staple cavity 88100 above the deck surface 88010. As the staple stored in the staple cavity 88100 is ejected, or fired, during a staple firing actuation, the legs of the staple are supported by the projections 88150 and 88160 when the staple legs emerge above the deck surface 88010. In such instances, the possibility of the staple legs becoming misaligned with the staple forming pockets defined in the anvil is reduced. Further to the below, referring to FIGS. 7-9, projections 89110, 89210, and 89310 also comprise staple cavity extenders.

As discussed further below, it is desirable to maintain the tissue captured between the jaws of the end effector in position during the staple firing stroke to optimize the sealing and cutting of the tissue. In various instances, it is desirable to hold the tissue positioned adjacent the longitudinal slot of the cartridge body as this tissue may experience the greatest displacement forces during the staple firing stroke owing to the firing driver translating distally through the longitudinal slot. Such displacement forces tend to lead to rippling and/or bunching of the tissue that can result in a non-uniform staple formation line and/or an ineffective seal along the cut line, for example. Moreover, while projections extending from the cartridge deck can be used to hold the tissue in position, such projections can create stress and strain within the tissue and, as discussed in greater detail below, the projections can be configured to alleviate or reduce the stress and strain within the tissue.

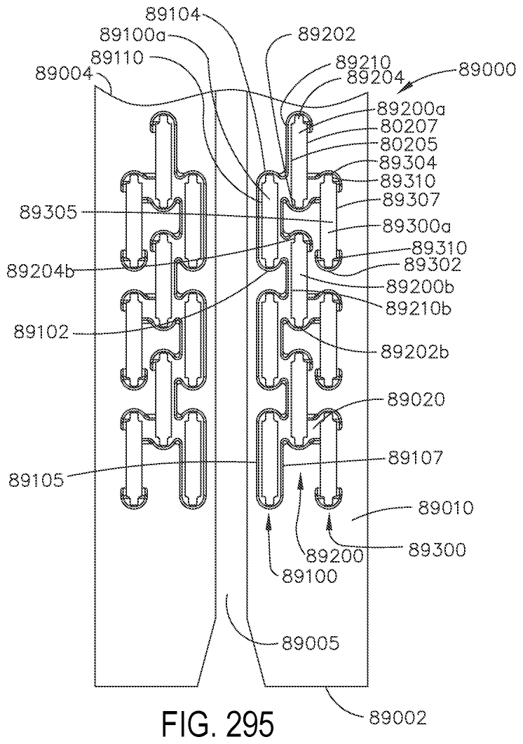

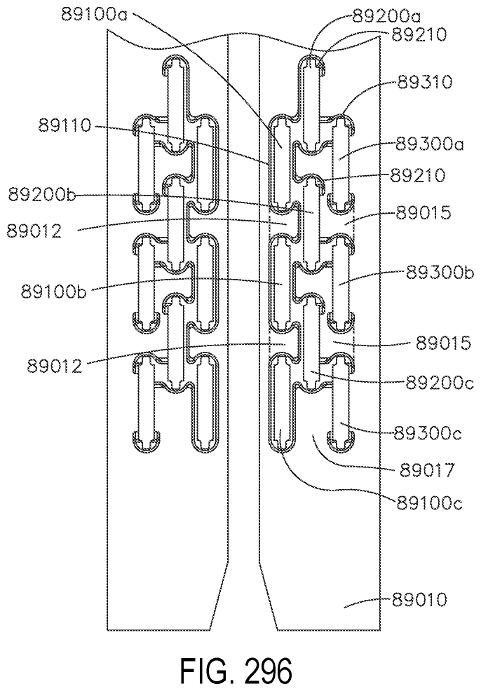

Referring to FIGS. 295 and 296, a staple cartridge 89000 comprises a cartridge body including a deck surface 89010. A longitudinal slot 89005 is defined in the cartridge body and extends from a proximal end 89002 toward a distal end 89004 of the staple cartridge 89000. The longitudinal slot 89005 is sized to accommodate the travel of a firing driver through the staple cartridge 89000 during a staple firing stroke.

Staple cavities are defined in the staple cartridge 89000 and are arranged in six longitudinal rows. Three longitudinal rows of staple cavities are defined on a first side of the longitudinal slot 89005, and three longitudinal rows of staple cavities are defined on a second side of the longitudinal slot 89005. The arrangement of staple cavities on the first side is mirrored across the longitudinal slot 89005 onto the second side of the staple cartridge 89000. In alternative embodiments, any suitable arrangement of staple cavities can be used. As shown in FIGS. 295 and 296, a first longitudinal row of staple cavities 89100 extends alongside the longitudinal slot 89005, a second longitudinal row of staple cavities 89200 extends alongside the first longitudinal row of staple cavities 89100, and a third longitudinal row of staple cavities 89300 extends alongside the second longitudinal row of staple cavities 89200. The first, second, and third longitudinal rows of staple cavities 89100, 89200, 89300 are defined on a first side of the longitudinal slot 89005.

A staple cavity from the first longitudinal row of staple cavities 89100 comprises a corresponding first projection 89110 extending from the deck surface 89010. Each first projection 89110 surrounds the staple cavity 89100 from the first longitudinal row to a first degree. Stated another way, each first projection 89110 surrounds a first length of a perimeter of a staple cavity 89100 from the first longitudinal row. As shown in FIG. 295, the first projection 89110 surrounds a staple cavity 89100 from the first longitudinal row in its entirety. Stated another way, the first projection 89110 surrounds a proximal end 89102, a distal end 89104, and the intermediate portions of the staple cavity sidewalls 89105, 89107 extending between the proximal end and the distal end of the staple cavity 89100. As shown, the first projection 89110 continuously surrounds a staple cavity 89100 from the first longitudinal row; however, it is also envisioned that numerous discrete projections can cooperate to surround the individual staple cavity 89100 in the first longitudinal row. Projections akin to the first projection 89110 surround all of the staple cavities in the first longitudinal row 89100.

A staple cavity from the second longitudinal row of staple cavities 89200 comprises a corresponding second projection 89210 extending from the deck surface 89010. Each second projection 89210 surrounds a staple cavity 89200 from the second longitudinal row to a second degree. The second degree is less than the first degree surrounded by the first projections 89110 in the first longitudinal row of staple cavities 89100. Stated another way, each second projection 89210 surrounds a second length of a perimeter of a staple cavity 89200 from the second longitudinal row. The second length is less than the first length. As shown, each second projection 89210 surrounds a proximal end 89202 and a distal end 89204 of the staple cavity 89200. Each second projection 89210 further surrounds an intermediate portion extending between the proximal end 89202 and the distal end 89204 on a first lateral side 89205 of the staple cavity 89200; however, as depicted, each second projection 89210 does not surround an intermediate portion extending between the proximal end 89202 and the distal end 89204 on a second lateral side 89207 of the staple cavity 89200. Stated another way, the second projection 89210 forms a C-shaped profile around the staple cavities in the second longitudinal row 89200. The second projection 89210 surrounds the lateral side of the staple cavity 89200 that is positioned closer to the longitudinal slot 89005 than the opposing lateral side. As shown, the second projection 89210 continuously surrounds a staple cavity 89200 from the second longitudinal row; however, it is also envisioned that numerous discrete projections can cooperate to surround the individual staple cavity 89200 in the second longitudinal row. Projections akin to the second projection 89210 surround all staple cavities in the second longitudinal row 89200.

A staple cavity from the third longitudinal row of staple cavities 89300 comprises a corresponding third projection 89310. Each third projection 89310 surrounds a staple cavity 89300 from the third longitudinal row to a third degree. The third degree is less than the first degree surrounded by the first projections 89110 in the first longitudinal row of staple cavities 89100, and the third degree is less than the second degree surrounded by the second projections 89210 in the second longitudinal row of staple cavities 89200. Stated another way, each third projection 89310 surrounds a third length of a perimeter of a staple cavity 89300 from the third longitudinal row. The third length is less than the second length and the first length. As shown, the third projection 89310 surrounds a proximal end 89302 and a distal end 89304 of the staple cavity 89300. Intermediate portions extending between the proximal end 89302 and the distal end 89304 are left unsurrounded. Stated another way, the third projection 89310 surrounds only the proximal and distal ends of staple cavities in the third longitudinal row 89300. Projections akin to the third projection 89310 surround all staple cavities in the third longitudinal row 89300.

As depicted in FIG. 295, the projections 89110, 89210, 89310 from each of the three longitudinal rows of staple cavities 89100, 89200, 89300 are interconnected with one another. Such interconnections serve to improve tissue grip and compression, for example. Such interconnections are extend less than two-thirds the length of one lateral side of a staple cavity; however, in alternative embodiments, the interconnections can extend any suitable length. As discussed above, a first staple cavity 89100a from the first longitudinal row 89100 comprises a proximal end 89102 and a distal end 89104. A first staple cavity 89200a from the second longitudinal row 89200 comprises a proximal end 89202 and a distal end 89204, and a second staple cavity 89200b from the second longitudinal row 89200 comprises a proximal end 89202b and a distal end 89204b. A first staple cavity 89300a from the third longitudinal row 89300 comprises a proximal end 89302 and a distal end 89304.

The first projection 89110 surrounding the first staple cavity 89100a from the first longitudinal row 89100 is continuously coupled to, or otherwise integral with, the second projection 89210 surrounding the first staple cavity 89200a from the second longitudinal row and the second staple cavity 89200b from the second longitudinal row 89200. More specifically, the first projection 89110 is coupled to the second projection 89210 surrounding the first staple cavity 89200a from the second longitudinal row 89200 at a distal end 89204 of the first staple cavity 89100a from the first longitudinal row and a proximal end 89202 of the first staple cavity 89200a from the second longitudinal row 89200. The first projection 89110 is further coupled to the second projection 89210 surrounding the second staple cavity 89200b from the second longitudinal row 89200 at a proximal end 89202 of the first staple cavity 89100a from the first longitudinal row 89100 and a distal end 89204b of the second staple cavity 89200b from the second longitudinal row 89200.

The second projection 89210 surrounding the first staple cavity 89200a from the second longitudinal row 89200 is continuously coupled to, or otherwise integral with, the third projection 89310 surrounding the distal end 89304 of the first staple cavity 89300a from the third longitudinal row 89300. While the third projection 89310 surrounding the proximal end 89304 of the first staple cavity 89300a from the third longitudinal row 89300 is shown discrete from, or otherwise independent of, surrounding projections, it is envisioned the third projection 89310 surrounding the proximal end 89304 could be coupled to the second projection 89210 surrounding the distal end 89204b of the second staple cavity 89200b from the second longitudinal row 89200. Such described interconnections between the projections are repeated along the longitudinal length of the staple cartridge 89000 and mirrored across the longitudinal slot 89005.

As described above, each first projection 89110 at least partially surrounds a first staple cavity 89100 along a first perimeter length of the first staple cavity 89100, each second projection 89210 at least partially surrounds a second staple cavity 89200 along a second perimeter length of the second staple cavity 89200, and each third projection 89310 at least partially surrounds a third staple cavity 89300 along a third perimeter length of the third staple cavity 89300. As also described above, the first perimeter length is longer than the second perimeter length and the second perimeter length is longer than the third perimeter length. Owing to the longer the first perimeter length of the first projections 89100, the first projections 89110 can have a larger contact area with the patient tissue positioned against the deck surface 89010 than the second projections 89200 and the third projections 89300. The larger contact area provided by the first projections 89110 can provide the staple cartridge 89000 with a greater control over the patient tissue around the first staple cavities 89100 as compared to the second staple cavities 89200 and the third staple cavities 89300. As discussed above, the firing driver cuts the patient tissue adjacent the first staple cavities 89100 and providing a high degree of control over the patient tissue over and around the first staple cavities 89100 can reduce, for example, the possibility of the patient tissue bunching up in front of the firing driver and/or the firing driver tearing the patient tissue. Likewise, the second projections 89210 can have a larger contact area with the patient tissue positioned against the deck surface 89010 than the third projections 89310 and, as a result, the staple cartridge 89000 can provide greater control over the patient tissue around the second staple cavities 89200 as compared to the third staple cavities 89300. Moreover, as a result of the above, the patient tissue over and around the third staple cavities 89300 may experience less stress and strain than the patient tissue over and around the second staple cavities 89200 and the first staple cavities 89100. Similarly, as a result of the above, the patient tissue over and around the second staple cavities 89200 may experience less stress and strain the patient tissue over and around the first staple cavities 89100. As a result of the above, the patient tissue can be tightly controlled over and around the first staple cavities 89100, albeit with a high amount of stress and strain within that region of the patient tissue, and more loosely controlled over and around the third staple cavities 89300 resulting in a lower amount of stress and strain within that region of the patient tissue.

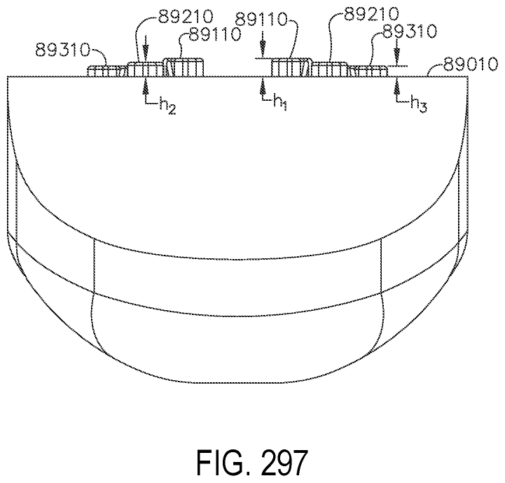

In addition to the above, the control over the patient tissue can be improved by varying the height to which each projection extends from the deck surface 89010. As shown in FIG. 297, the first projections 89110 extend from the deck surface 89010 to a first height h1, the second projections 89210 extend from the deck surface 89010 to a second height h2, and the third projections 89310 extend from the deck surface 89010 to a third height h3. The first height h1 is greater than the second height h2 and the third height h3. The second height h2 is greater than the third height h3. The additional support provided to staples in the first longitudinal row 89100 by having taller projections 89110 improves the likelihood of forming a tighter staple along the cut line, as the increased support enables more staple leg forming distance by maintaining the staple in an optimal upright orientation. In the optimal upright orientation, the staples are deployed substantially perpendicular to the deck surface 89010 during the staple firing stroke. When the staples are maintained in the optimal upright orientation, the formation of such staples results in a desired, rounded “B” shape.

In various embodiments, as discussed above, the staple cartridge 89000 is intended to be seated in a channel of an end effector. The staple cartridge 89000 and the channel are configured to oppose an anvil of the end effector, and the end effector is configured to be moved into a fully-closed configuration to clamp target tissue between the anvil and the staple cartridge 89000. When the end effector is in the fully-closed position, a first tissue gap is defined between the first projections 89110 and a cartridge-facing surface of the anvil. A second tissue gap is defined between the second projections 89210 and the cartridge-facing surface of the anvil. A third tissue gap is defined between the third projections 89310 and the cartridge-facing surface of the anvil. Due at least in part to the first height h1 of the first projections 89110 being greater than the second height h2 of the second projections 89210 and the third height h3 of the third projections 89310, the first tissue gap is smaller than the second tissue gap and the third tissue gap. Due at least in part to the second height h2 of the second projections 89210 being greater than the third height h3 of the third projections 89310, the second tissue gap is smaller than the third tissue gap. Providing a smaller tissue gap along the first longitudinal row 89100 results in a greater tissue compression adjacent the longitudinal slot 89005. Such greater tissue compression adjacent the longitudinal slot 89005 serves to maintain the tissue closest to the longitudinal slot 89005 in position during a staple firing stroke, for example. Decreasing the tissue compression laterally away from the longitudinal slot 89005 further allows for the flowable contents of the tissue, such as blood, for example, to flow laterally away from the cut line as the tissue is compressed. Moreover, the presence of projections should be weighed against any potential damage to the adjacent tissue. In addition, the frequency of the projections and/or the geometry of the projections, for example, should also be weighed.

In various other embodiments, each projection extends from the deck surface 89010 to a single, uniform height.

Further to the above, the deck surface 89010 comprises a recessed valley, or a plurality of valleys, amongst the projections 89110, 89210, 89310. As the patient tissue is compressed between the staple cartridge 89000 and the opposing anvil, the patient tissue conforms, flows, and/or or otherwise moves into the recessed valleys. To minimize tissue damage, and/or reduce stress and strain within the patient tissue, for example, it is desirable to increase the surface area of the deck surface 89010, or valleys, between surrounding projections. Increasing this surface area reduces the pressure experienced by the tissue as it conforms around the projections and into the valley areas. A minimum ideal deck surface, or valley, area can be calculated by considering the heights of the projections, the widths of the projections, and/or the pressure induced on the adjacent tissue by the projections. In various embodiments, an optimal surface area of the valleys is double the area of the surrounding projections. In at least one embodiment, an optimal surface area of the valleys is at least double the area of the surrounding projections. In at least one embodiment, an optimal surface area of the valleys is triple the area of the surrounding projections.

Referring to FIG. 296, local valleys 89012, 89015, 89017 are defined on the deck surface 89010 by surrounding projections. More specifically, a first staple cavity 89100a from a first longitudinal row, a second staple cavity 89100b from the first longitudinal row, and a second staple cavity 89200b from a second longitudinal row surround a first valley 89012. The first valley 89012 is further bounded by the local deck surface, or a first valley floor. The first valley 89012 comprises a first open end that faces, or otherwise opens, toward the longitudinal slot 89005. The first valley 89012 further comprises a first closed, or bounded end.

The first staple cavity 89100a from the first longitudinal row, a first staple cavity 89200a from the second longitudinal row, the second staple cavity 89200b from the second longitudinal row, and a first staple cavity 89300a from a third longitudinal row surround a second valley 89015. The second valley 89015 is S-shaped or Z-shaped, for example, but can comprise any suitable configuration. Moreover, the second valley 89015 is circuitous and comprises at least one laterally-extending region and at least one longitudinally-extending region. An intermixing of laterally-extending regions and longitudinally-extending regions in a valley can provide both lateral and longitudinal control over the flow of patient tissue. The second valley 89015 is further bounded by the local deck surface, or a second valley floor. The second valley 89015 comprises a second open end that opposes, or otherwise opens away from, the longitudinal slot 89005. The second valley 89015 further comprises a second closed, or bounded end. The projections 89110, 89210, 89310 that surround such cavities 89100a, 89200a, 89200b, 89300a extend from the deck surface 89010 and define a perimeter of the valley 89015.

The area of the second valley 89015 is greater than the area of the local surrounding projection area. The local surrounding projection area of the valley 89015 includes the area of anvil-facing surfaces of the projections 89110, 89210, 89310 surrounding the staple cavities 89100a, 89200a, 89200b, 89300a. While the area of the valley 89015 is ideally larger than the local surrounding projection area, an optimal area of the valley 89015 can be reduced by one-fourth for each ratio of tissue gap to projection height quartile. For example, a tissue gap of 0.100″ is defined between the deck surface 89010 and a cartridge-facing surface of an opposing anvil when the end effector is in a fully-closed position. In instances where the heights of the projections are 0.025″, the optimal area of the valley 89015 can be reduced by one-fourth, as the heights of the projections extend one-fourth of the tissue gap. In instances where the heights of the projections are 0.050″, the optimal area of the valley 89015 can be reduced by one-half, as the heights of the projections extend one-half of the tissue gap.

A third valley 89017 is defined on the deck surface 89010 and is at least partially bounded by a third staple cavity 89100c from the first longitudinal row, a third staple cavity 89200c from the second longitudinal row, and a third staple cavity 89300c from the third longitudinal row. Stated another way, the third valley 89017 is at least partially bounded by the proximal-most staple cavities from the first longitudinal row, the second longitudinal row, and the third longitudinal row. The third valley 89017 is further bounded by the local deck surface, or a third valley floor. The third valley 89017 comprises a third bounded end, and a third open end, wherein the third open end faces, or otherwise opens, toward the proximal end 89002 of the staple cartridge 89000.

In addition to or in lieu of holding the tissue in place during the staple firing stroke, in various embodiments, the projections extending from the cartridge deck can grip and/or secure an implantable adjunct, such as a layer of buttress material, for example, against the cartridge deck. Moreover, in various embodiments, the projections, especially the projections that at least partially surround the staple cavities, can secure the adjunct in position as staples are ejected from the cavities and/or as the adjunct is severed during a staple firing stroke. Moreover, in various embodiments, the projections that are adjacent the staple cavities can be configured to guide and orient the staples as the staples are fired, or ejected, from the cartridge body during the staple firing stroke.

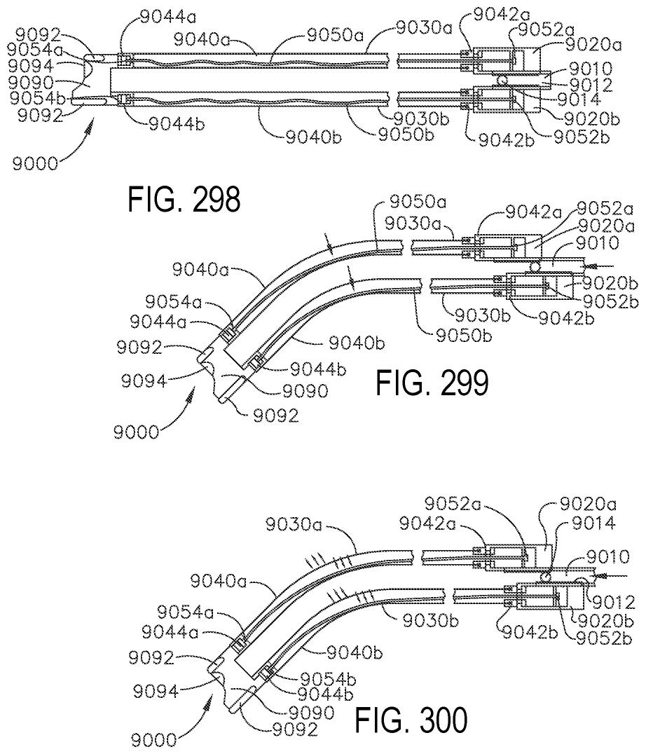

A firing drive system 9000 is illustrated in FIGS. 298-300. The firing drive system 9000 comprises a firing beam, or driver, 9090 that is pushed distally during a staple firing stroke. As discussed in greater detail below, the firing drive system 9000 is configured to push the firing driver 9090 through a staple cartridge to perform a staple firing stroke. The firing drive system 9000 comprises a translatable drive rack 9010 and an electric motor comprising an output engaged with the translatable rack 9010. The translatable rack 9010 comprises a shaft 9012 and a rotatable pinion gear 9014 rotatably mounted to the shaft 9012. The firing drive system 9000 further comprises a first slider 9020a that includes a linear, or longitudinal, rack of teeth operably intermeshed with the pinion gear 9014 and a second slider 9020b that includes a linear, or longitudinal, rack of teeth operably intermeshed with the pinion gear 9014 positioned on an opposite side of the pinion gear 9014. The firing drive system 9000 further comprises a first drive rod assembly 9030a coupled to and extending from the first slider 9020a and a second drive rod assembly 9030b coupled to and extending from the second slider 9020b. The first drive rod assembly 9030a comprises a push coil 9040a and a cable 9050a extending through the push coil 9040a. The push coil 9040a comprises a wound coil wire including a proximal end 9042a mounted to the first slider 9020a and a distal end 9044a mounted to a tissue cutting head 9090. The wound coil wire of the push coil 9040a has a longitudinal aperture defined therein extending between the proximal end 9042a and the distal end 9044a. The cable 9050a extends through the longitudinal aperture and comprises a proximal end 9052a mounted to the first slider 9020a and a distal end 9054a mounted to the tissue cutting head 9090. The second drive rod assembly 9030b comprises a push coil 9040b and a cable 9050b extending through the push coil 9040b. The push coil 9040b comprises a wound coil wire including a proximal end 9042b mounted to the second slider 9020b and a distal end 9044b mounted to the firing driver 9090. The wound coil wire of the push coil 9040b has a longitudinal aperture defined therein extending between the proximal end 9042b and the distal end 9044b. The cable 9050b extends through the longitudinal aperture of the push coil 9040b and comprises a proximal end 9052b mounted to the second slider 9020b and a distal end 9054b mounted to the firing driver 9090. The cables 9050a and 9050b are comprised of stainless steel and/or manufactured fibers spun from liquid-crystal polymer, such as Vectran which is manufactured by Kuraray Company, Limited, for example. As discussed below, the cables 9050a and 9050b can support the push coils 9040a and 9040b, respectively, during the staple firing stroke.

As discussed above, the firing drive system 9000 is operable to move the firing driver 9090 distally to perform a staple firing stroke. During the staple firing stroke, a firing motor, for example, pushes the rack 9010 distally which pushes the first and second sliders 9020a, 9020b and the first and second drive rod assemblies 9030a, 9030b distally via the pinion gear 9014. Also during the staple firing stroke, the drive rod assemblies 9030a, 9030b push the firing driver 9090 through a staple cartridge such that the firing driver 9090 engages a sled in the staple cartridge and pushes the sled distally to eject the staples from the staple cartridge. The firing driver 9090 comprises a tissue cutting knife 9094 configured to incise the patient tissue being stapled as the firing driver 9090 is advanced distally. However, other embodiments are envisioned in which the tissue cutting knife 9094 is not integral with the firing driver 9090 and instead comprises a separate component pushed distally by the firing driver 9090. The firing driver 9090 further comprises cams 9092 that engage the jaws of the end effector and the hold the jaws in position during the staple firing stroke. When the end effector is in an unarticulated position during the staple firing stroke, referring to FIG. 298, the first slider 9020a and the second slider 9020b are aligned with one another. In various instances, referring to FIG. 299, the end effector is in an articulated position, i.e., in a rotated position relative to a longitudinal axis of the stapling instrument shaft, during the staple firing stroke. In such instances, referring to FIG. 300, the firing drive system 9000 moves around a bend created by an articulation joint rotatably connecting the end effector to the shaft. As can be seen in FIGS. 299 and 300, the drive rod assemblies 9030a and 9030b are flexible and bend to accommodate the articulation of the end effector. In this articulated configuration, the first drive rod assembly 9030a extends along a first radius and the second drive rod assembly 9030b extends along a second radius that is different than the first radius. Stated another way, the path of the first drive rod assembly 9030a during the staple firing stroke is longer than the path of the second drive rod assembly 9030b. To accommodate this difference, as can be seen in FIG. 300, the first slider 9020a has shifted distally and the second slider 9020b has shifted proximally.

Further to the above, referring again to FIG. 298, the cable 9050a extending through the push coil 9040a of the first drive rod assembly 9030a has slack when the end effector is in its unarticulated configuration. In such instances, the cable 9050a provides little, if any, support to the push coil 9040a. Similarly, the cable 9050b extending through the push coil 9040b of the second drive rod assembly 9030b has slack when the end effector in its unarticulated configuration. In such instances, the cable 9050b provides little, if any, support to the push coil 9050b. When the end effector is in articulated configuration, referring to FIGS. 299 and 300, the cables 9050a and 9050b are pulled taut within their respective push coils 9040a and 9040b. In such instances, the cables 9050a and 9050b come into contact with the sides of their respective push coils 9040a and 9040b and support the push coils 9040a and 9040b in their flexed state. As a result, the cables 9050a and 9050b can prevent, or at least reduce the possibility of, the push coils 9040a and 9040b buckling during the staple firing stroke.

As discussed above, the drive rod assemblies 9030a and 9030b are pushed distally to perform the staple firing stroke. Further to the above, the first drive rod assembly 9030a moves through an anvil jaw of the end effector during the staple firing stroke. Similarly, the second drive rod assembly 9030b moves through a cartridge jaw of the end effector during the staple firing stroke. In certain embodiments, the drive rod assemblies 9030a and 9030b are advanced distally to move the firing driver 9090 into contact with the anvil jaw and push the anvil jaw closed during a closure stroke. The anvil jaw is configured to support the first drive rod assembly 9030a during the closure stroke and the staple firing stroke. The anvil jaw comprises a longitudinal aperture defined therein having at least one sidewall that supports the push coil 9040a and prevents the push coil 9040a from buckling, or at least reduces the possibility of the push coil 9040a buckling. In various embodiments, the longitudinal aperture in the anvil jaw is defined between a frame and a cap welded to the frame. In at least one such embodiment, the cap extends along a longitudinal axis of the anvil jaw between a proximal end and a distal end wherein the cap is thicker at the proximal end than the distal end. In such embodiments, the proximal end of the longitudinal aperture is narrower than the distal end and can provide more, or closer, support to the push coil 9040a than the distal end. As a result, the proximal end of the longitudinal aperture can closely support the push coil 9040a during the closure stroke and then throughout the staple firing stroke. In at least one embodiment, the drive rod assemblies 9030a and 9030b are advanced distally to move the firing driver 9090 into contact with the cartridge jaw and push the cartridge jaw closed during a closure stroke. The cartridge jaw and/or the staple cartridge seated in the cartridge jaw can comprise a longitudinal aperture configured to support the drive rod assembly 9030b during the closure stroke and the staple firing stroke.

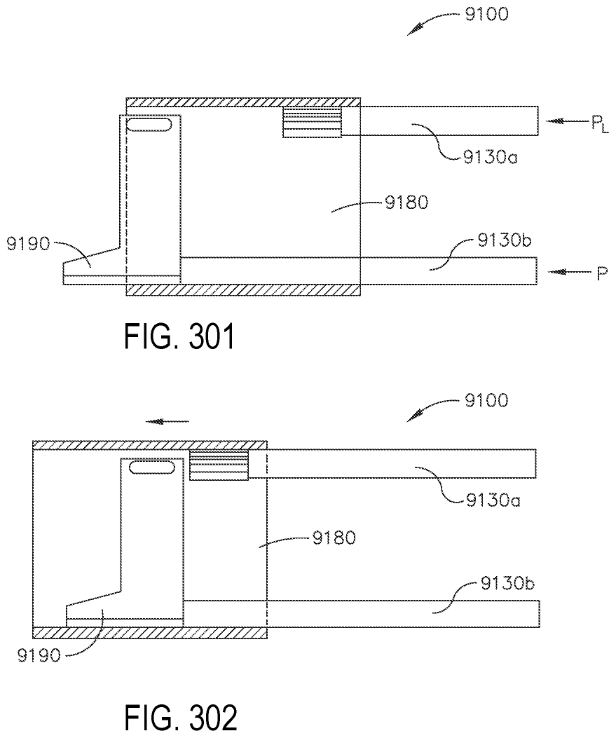

Referring to FIGS. 301 and 302, a drive system 9100 of a surgical stapling instrument comprises a closure driver 9180 and a firing driver 9190. The closure driver 9180 is movable distally by a closure drive during a closure stroke to engage and move the jaws of an end effector from an open configuration to a closed configuration. The closure drive comprises a push coil 9130a mounted to the closure driver 9180 that is moved distally by an electric motor of the closure drive. In various other embodiments, the closure drive comprises a handcrank that is operable to drive the push coil 9130a distally. The push coil 9130a comprises a wound metal wire and includes a longitudinal aperture defined therein. The wound metal wire comprises coils that are in contact with one another in a coil stack and co-operatively form a pushable yet flexible rod that can bend or flex within an articulation joint. Similar to the above, the closure drive can further include a wire extending through the longitudinal aperture of the push coil 9130a that can support the push coil 9130a, especially in the bent or flexed portion of the push coil 9130a, and prevent the push coil 9130a from buckling, or at least reduce the possibility of the push coil 9130a buckling. To open the jaws of the end effector, the closure drive is operated in reverse to pull the push coil 9130a and the closure driver 9180 proximally. The coil stack defining the push coil 9130a is sufficiently rigid to apply a pulling force to the closure driver 9180.

The firing driver 9190 is movable distally by a firing drive during a staple firing stroke to staple and incise patient tissue. The firing drive comprises a push coil 9130b mounted to the firing driver 9190 that is moved distally by an electric motor of the firing drive. In various other embodiments, the firing drive comprises a handcrank that is operable to drive the push coil 9130b distally. The push coil 9130b comprises a wound metal wire and includes a longitudinal aperture defined therein. The wound metal wire comprises coils that are in contact with one another in a coil stack and co-operatively form a pushable yet flexible rod that can bend or flex within an articulation joint. Similar to the above, the firing drive can further include a wire extending through the longitudinal aperture of the push coil 9130b that can support the push coil 9130b, especially in the bent or flexed portion of the push coil 9130b, and prevent the push coil 9130b from buckling, or at least reduce the possibility of the push coil 9130b buckling. After the staple firing stroke, the firing drive is operated in reverse to pull the push coil 9130b and the firing driver 9190 proximally. The coil stack defining the push coil 9130b is sufficiently rigid to apply a pulling force to the firing driver 9190.

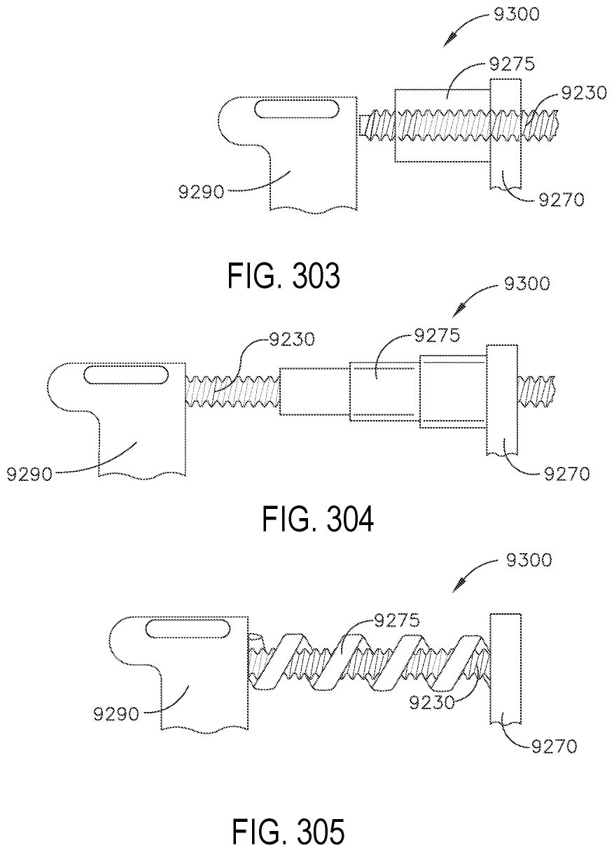

Referring to FIGS. 303 and 304, a firing drive 9200 of a surgical stapling instrument comprises a firing driver 9290 and a push coil 9230 mounted to the firing driver 9290 that is moved distally by an electric motor of the firing drive 9200 during the staple firing stroke. In various other embodiments, the firing drive 9200 comprises a handcrank that is operable to drive the push coil 9230 distally. In either event, the push coil 9230 comprises a wound metal wire and includes a longitudinal aperture defined therein. The wound metal wire comprises coils that are in contact with one another in a coil stack and co-operatively form a pushable yet flexible rod that can bend or flex within an articulation joint. Similar to the above, the firing drive 9200 can further include a wire extending through the longitudinal aperture of the push coil 9230 that can support the push coil 9230 from the inside, especially in the bent or flexed portion of the push coil 9230, and prevent the push coil 9230 from buckling, or at least reduce the possibility of the push coil 9230 buckling. In addition to or in lieu of the internal wire, the firing drive 9200 further comprises an external telescoping support 9275 configured to support the push coil 9230 from the outside and prevent the push coil 9230 from buckling, or at least reduce the possibility of the push coil 9230 buckling. The telescoping support 9275 comprises a first end mounted to a frame 9270 of the surgical stapling instrument and a second end mounted to the push coil 9230. When the push coil 9230 is advanced distally during the staple firing stroke, the telescoping support 9275 moves from a contracted configuration (FIG. 303) to an extended configuration (FIG. 304). In such instances, the push coil 9230 pulls the second end of the telescoping support 9275 distally relative to the first end as the push coil 9230 is advanced distally which, as a result, stretches the telescoping support 9275. The telescoping support 9275 comprises an internal longitudinal aperture surrounding, or at least partially surrounding, the push coil 9230 that is sized and configured such that the sidewall of the internal aperture supports and limits the lateral deflection of the push coil 9230. After the staple firing stroke, the firing drive is operated in reverse to pull the push coil 9230 and the firing driver 9290 proximally. The coil stack defining the push coil 9230 is sufficiently rigid to apply a pulling force to the firing driver 9290. Moreover, in such instances, the push coil 9230 moves the second end of the telescoping support 9275 proximally toward the first end to contract the telescoping support 9275 back into is contracted configuration.

Referring to FIG. 305, a firing drive 9300 of a surgical stapling instrument comprises a firing driver 9390 and a push coil 9330 mounted to the firing driver 9390 that is moved distally by an electric motor of the firing drive 9300 during the staple firing stroke. In various other embodiments, the firing drive 9300 comprises a handcrank that is operable to drive the push coil 9330 distally. In either event, the push coil 9330 comprises a wound metal wire and includes a longitudinal aperture defined therein. The wound metal wire comprises coils that are in contact with one another in a coil stack and co-operatively form a pushable yet flexible rod that can bend or flex within an articulation joint. Similar to the above, the firing drive 9300 can further include a wire extending through the longitudinal aperture of the push coil 9330 that can support the push coil 9330 from the inside, especially in the bent or flexed portion of the push coil 9330, and prevent the push coil 9330 from buckling, or at least reduce the possibility of the push coil 9330 buckling. In addition to or in lieu of the internal wire, the firing drive 9300 further comprises an extendable outer support 9375 configured to support the push coil 9330 from the outside and prevent the push coil 9330 from buckling, or at least reduce the possibility of the push coil 9330 buckling. The extendable outer support 9375 comprises a helically-wound wire comprising a first end mounted to a frame 9370 of the surgical stapling instrument and a second end mounted to the firing driver 9390. When the push coil 9370 is advanced distally during the staple firing stroke, the extendable outer support 9375 moves from a contracted coiled configuration to an extended coil configuration (FIG. 305). In such instances, the firing driver 9390 pulls the second end of the support 9375 distally relative to the first end as the firing driver 9390 is advanced distally which, as a result, stretches the support 9375. The extendable support 9375 comprises an internal longitudinal aperture surrounding, or at least partially surrounding, the push coil 9330 that is sized and configured such that the sidewall of the internal aperture supports and limits the lateral deflection of the push coil 9330. After the staple firing stroke, the firing drive is operated in reverse to pull the push coil 9330 and the firing driver 9390 proximally. The coil stack defining the push coil 9330 is sufficiently rigid to apply a pulling force to the firing driver 9390. Moreover, in such instances, the push coil 9330 moves the second end of the extendable support 9375 proximally toward the first end to contract the support 9375 back into is contracted configuration.

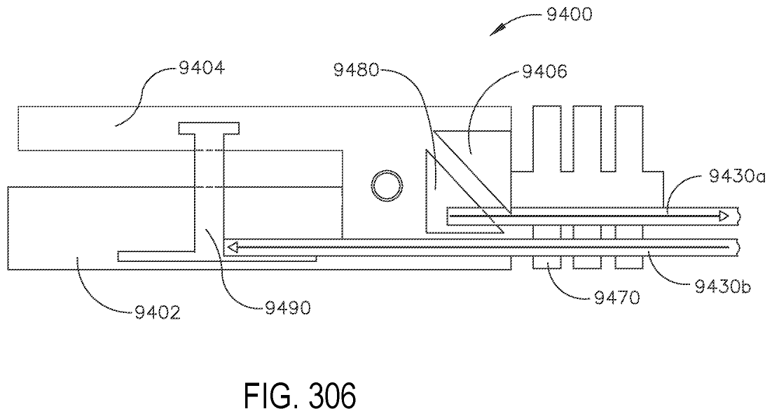

Referring to FIG. 306, a drive system 9400 of a surgical stapling instrument comprises a closure driver 9480 and a firing driver 9490. The closure driver 9480 is movable proximally by a universal closing-firing drive during a closure stroke to engage a cam surface 9406 defined on an anvil jaw 9404 of the surgical stapling instrument to move the anvil jaw 9404 relative to a staple cartridge jaw 9402 of the surgical stapling instrument from an open position to a closed position. A push coil 9430a is mounted to the closure driver 9480 that is moved proximally by an electric motor of the universal drive. The push coil 9430a comprises a wound metal wire and includes a longitudinal aperture defined therein. The wound metal wire comprises a coil stack that is sufficiently rigid to apply a pulling force to the closure driver 9480 yet flexible rod enough such that the closure driver 9480 can bend or flex within an articulation joint 9470. Similar to the above, the closure drive can further include a wire extending through the longitudinal aperture of the push coil 9430a that can support the push coil 9430a, especially in the bent or flexed portion of the push coil 9430a, and prevent the push coil 9430a from buckling, or at least reduce the possibility of the push coil 9430a buckling. To open the anvil jaw 9404, the universal drive is operated in reverse to push the push coil 9430a and the closure driver 9480 distally.

Further to the above, the firing driver 9490 is movable distally by the universal drive during a staple firing stroke to staple and incise patient tissue. The firing drive comprises a push coil 9430b mounted to the firing driver 9490 that is moved distally by the electric motor of the universal drive. In various embodiments, the push coil 9430a comprises a first nut threadably engaged with a threaded shaft of the universal drive and the push coil 9430b comprises a second nut threadably engaged with the threaded shaft. When the threaded shaft is rotated, both the push coils 9430a, 9430b are moved as described herein. The push coil 9430b comprises a wound metal wire and includes a longitudinal aperture defined therein. The wound metal wire comprises coils that are in contact with one another in a coil stack and co-operatively form a pushable yet flexible rod that can bend or flex within the articulation joint 9470. Similar to the above, the firing drive can further include a wire extending through the longitudinal aperture of the push coil 9430b that can support the push coil 9430b, especially in the bent or flexed portion of the push coil 9430b, and prevent the push coil 9430b from buckling, or at least reduce the possibility of the push coil 9430b buckling. After the staple firing stroke, the firing drive is operated in reverse to pull the push coil 9430b and the firing driver 9490 proximally. The coil stack defining the push coil 9430b is sufficiently rigid to apply a pulling force to the firing driver 9490.

As discussed above, referring again to FIGS. 22-24, the anvil jaw 3320 comprises tissue stops 3323 which inhibit the proximal migration of patient tissue into the proximal end of the end effector 3000. The anvil jaw 3320 is an assembly comprised of two components—a first component, or frame, including the tissue stops 3323, the tissue compression surface 3321, and two laterally-extending pivot pins that rotatably connect the anvil jaw 3320 to the cartridge jaw 3310—and a second component comprising a cap attached to the first component. The first component comprises a longitudinal channel defined in the top thereof, i.e., on the opposite side of the tissue compression surface 3321, that is configured to receive the second cam 3520 of the firing driver 3500 during the staple firing stroke and the second component is welded to the first component to substantially enclose the channel defined in the first component. The first and second components of the anvil jaw 3320 are comprised of stainless steel but can be comprised of any suitable metal. The tissue stops 3323 are sized and configured such that there is little, if any, gap between the tissue stops 3323 and the lateral sidewalls of the cartridge jaw 3310. As a result, the possibility of the patient tissue getting caught or pinched between the tissue stops 3323 and the lateral sidewalls of the cartridge jaw 3310 is reduced. In various embodiments, the tissue stops 3323 are not integrally formed with the first component of the anvil jaw 3320. Rather, in at least one such embodiment, the tissue stops 3323 are attached to the first component of the anvil jaw 3320 and are comprised of rubber and/or plastic, for example, configured to wipe against the lateral sidewalls of the cartridge jaw 3310. In at least one embodiment, each of the tissue stops 3323 comprises one or move beveled surfaces configured to push patient tissue laterally outwardly, or away, from the anvil jaw 3320 and the cartridge jaw 3310—including the gap therebetween—as the anvil jaw 3320 is being closed. As a result of this arrangement, the possibility of patient tissue being pinched between the anvil jaw 3320 and the lateral sidewalls of the cartridge jaw 3310 can be reduced.

As described above, the second component, or cap, of the anvil jaw 3320 substantially encloses the longitudinal channel defined in the top of the first component, or frame, of the anvil jaw 3320. The longitudinal channel comprises a longitudinal opening extending through the tissue compression surface 3321 that is configured to receive the firing driver 3500 as the firing driver 3500 moves distally during the staple firing stroke. The longitudinal opening extends longitudinally between six longitudinal rows of staple forming pockets defined in the tissue compression surface 3321—three longitudinal rows of staple forming pockets on one lateral side of the longitudinal opening and three longitudinal rows of staple forming pockets on the other lateral side of the longitudinal opening. The longitudinal channel comprises a substantially T-shaped profile, for example, extending longitudinally through the anvil jaw 3320. In some instances, tissue compressed between the tissue compression surface 3321 and the staple cartridge 2000, for instance, flows upwardly into the longitudinal channel and can block or impede the distal movement of the firing driver 3500. Also, in some instances, staples ejected from the staple cartridge 2000 can enter into the longitudinal opening—while being fired or after they have been fired—which can also block or impede the movement of the firing driver 3500 within the longitudinal channel. To address the above, in various embodiments, the second cam 3520 and the longitudinal channel are sized and configured such that there is little, if any, gap between the second cam 3520 and the walls defining the longitudinal channel. In addition to or in lieu of the above, the distal end of the second cam 3520, and/or any distally-facing surface of the firing driver 3500, comprises a wedge that pushes the tissue and/or staples downwardly out of the longitudinal channel during the staple firing stroke. In at least one such embodiment, the wedge comprises two angled surfaces extending laterally from a common longitudinal edge that are configured to push patient tissue both downwardly toward the staple cartridge and laterally away from the longitudinal opening of the longitudinal channel. In various instances, the distal end of the second cam 3520 comprises a snow plow or cow-catcher design, for example. In addition to or in lieu of the above, one or more materials are present in the longitudinal channel that block or inhibit patient tissue from flowing up into the longitudinal channel in front of the firing driver 3500. In at least one such embodiment, the material comprises a hemostatic material, for example, that is present in the longitudinal channel prior to the staple firing stroke, that inhibits staples from entering into the longitudinal channel during the staple firing stroke, and is pushed out of the longitudinal channel by the firing driver 3500 during the staple firing stroke onto the patient tissue. In addition to or in lieu of the above, a plunger is positioned within the longitudinal channel that is pushed distally by the firing driver 3500 during the staple firing stroke that cleans out the longitudinal channel ahead of the firing driver 3500. In at least one such embodiment, the plunger is comprised of rubber, plastic, and/or any suitable elastomeric material, for example, and has either an outer profile that matches the inner profile of the longitudinal channel in a line-to-line manner or an outer profile that is compressed inwardly by the sidewalls of the longitudinal channel. In either event, the plunger can be attached to the firing driver 9500 or can comprise a separate component that is unattached to the firing driver 9500.

In various instances, further to the above, the stapled patient tissue can flow upwardly into the longitudinal channel after the firing driver 3500 has passed thereby. In such instances, the stapled patient tissue can prevent or impede the proximal retraction of the firing driver 3500 after the staple firing stroke. To address this, the proximal end of the second cam 3520, and/or any proximally-facing surface of the firing driver 3500, comprises a wedge that pushes the patient tissue and/or staples downwardly out of the longitudinal channel as the firing driver 3500 is being retracted. In at least one such embodiment, the wedge comprises two angled surfaces extending laterally from a common longitudinal edge that are configured to push patient tissue both downwardly toward the staple cartridge and laterally away from the longitudinal opening of the longitudinal channel. In various instances, the proximal end of the second cam 3520 comprises a snow plow or cow-catcher design, for example. In at least one embodiment, the wedge is pivotally attached to the firing member 9500. In at least one embodiment, the pivotable wedge comprises a flap that is rotated laterally inwardly during the staple firing stroke and rotates laterally outwardly when the firing driver 9500 is being retracted. In at least one such embodiment, the drag force between the firing driver 9500 and the patient tissue causes the pivotable wedge to rotate or flare laterally outwardly and displace the patient tissue away from and/or out of the longitudinal channel in the anvil jaw 3520 as the firing member 9500 is being retracted. In addition to or in lieu of the above, a plunger is positioned within the longitudinal channel that is pushed proximally by the firing driver 3500 as the firing driver 3500 is being retracted that cleans out the longitudinal channel behind the firing driver 3500. In at least one such embodiment, the plunger is comprised of rubber, plastic, and/or any suitable elastomeric material, for example, and has either an outer profile that matches the inner profile of the longitudinal channel in a line-to-line manner or an outer profile that is compressed inwardly by the sidewalls of the longitudinal channel. The plunger is attached to the firing driver 9500, but need not be.

In various embodiments, further to the above, the firing driver 9500 comprises a distal head including the first and second cams 9510 and 9520 and an array of bands, or laminates, that push the distal head through the staple firing stroke. As the distal head is pushed distally, the bands of the firing driver 9500 can enter into the anvil jaw 3520 and/or the staple cartridge 2000. Stated another way, the bands of the firing driver 9500 can pass by the stapled and incised patient tissue during the staple firing stroke and as the firing driver 9500 is being retracted. In such instances, absent more, the edges of the distal head and/or the bands can catch on the stapled patient tissue which can prevent or impede the retraction of the firing driver 9500. In various embodiments, the back edges of the distal head are radiused to reduce the possibility of the distal head snagging on the patient tissue. In at least one such embodiment, each of the edges is radiused at least one half of a characteristic length of a surface defining the edge. In at least one embodiment, none of the proximal-facing surfaces of the distal head are flat. In various embodiments, the bands have a continuous tapered geometry between a distal end of the bands attached to the distal head and a proximal portion of the bands. In at least one embodiment, the bands have a distal portion attached to the distal head having a first height, a proximal portion having a second height that is shorter than the first height, and an intermediate portion extending between the proximal portion and the distal portion that has a height defined by one or more curvatures without a notch defined therein. Without notches, cutouts, and/or pockets defined in the bands of the firing driver 9500, the likelihood of the firing driver 9500 snagging on a staple in the patient tissue is reduced.

Further to the above, each of the staple forming pockets defined in the anvil jaw 3520 is positioned directly above a staple stored in the staple cartridge 2000 such that the staple, when fired, is moved along a deployment plane extending between the staple forming pocket and the stored staple. This deployment plane is parallel to the longitudinal axis of the staple cartridge 2000 and, also, parallel to the longitudinal axis of the anvil jaw 3520. In various instances, however, the staples will skew laterally inwardly and/or laterally outwardly as the staples are being fired-especially as the staples are passing through the patient tissue. In some instances, the skew is so significant that the legs of a staple may entirely miss a staple forming pocket defined in the anvil jaw 3520. Such staples may still be deformed against the anvil jaw 3520 nonetheless and assume an acceptable formed shape. In some instances, however, skewed staples in the innermost staple rows may enter the longitudinal opening defined in the anvil jaw 3520 which can block or impede the motion of the firing driver 9500. To reduce the possibility of this occurring, in at least one embodiment, the deployment planes of the staples in the innermost rows of staple cavities are oriented, or angled, laterally away from the longitudinal opening defined in the anvil jaw 3520 that is configured to receive the firing driver 9500. In various embodiments, the angle is 1 degree laterally outward, 3 degrees laterally outward, 5 degrees laterally outward, between 1 degree and 5 degrees laterally outward, 7 degrees laterally outward, 10 degrees laterally outward, between 5 degrees and 10 degrees laterally outward, 12 degrees laterally outward, and between 10 degrees and 15 degrees laterally outward, for example. The deployment planes of the staples in the intermediate longitudinal staple rows and/or the outermost longitudinal staple rows can also be angled laterally outwardly or, in alternative embodiments, the deployments planes of the staples in the intermediate longitudinal staple rows and the outermost longitudinal staple rows are not angled laterally outwardly.

In various embodiments, it is desirable for the staples to be deformed into a formed shape within the deployment plane. Such formed shapes can be referred to as two-dimensional formed shapes even though, as the reader will appreciate, the staples have a lateral thickness, i.e., a third dimension, at least equal to the wire diameter of the staples. In other embodiments, it is desirable for the legs of the staples to be deformed such that the legs are twisted to the sides of the deployment plane during the staple forming process. Such formed shapes can be referred to as three-dimensional formed shapes. In various instances, it is possible for the legs of a staple to twist out of its deployment plane as it is being deformed against the anvil jaw 3520. Such twisting can be acceptable in many instances; however, in some instances, the staple legs of the staples in the innermost longitudinal rows of staple cavities can twist into the longitudinal opening defined in the anvil jaw 3520 which can block or impede the movement of the firing driver 9500. To reduce the possibility of this occurring, in various embodiments, the staple forming pockets defined in the anvil jaw 3520 are configured to twist the legs of the staples in the innermost longitudinal row of staple cavities laterally outward out of their deployment planes. The staple forming pockets that deform the staples in the intermediate longitudinal rows of staple cavities and/or the staples in the outermost longitudinal rows of staple cavities are also configured to twist the staple legs out their respective staple deployment planes. In other embodiments, the staple forming pockets that deform the staples in the intermediate longitudinal rows of staples cavities and the staples in the outermost longitudinal rows of staple cavities are not configured to twist the staple legs out of their respective staple deployment planes.

In various surgical procedures, patient tissue, such as the stomach, for example, is stapled and incised along a treatment path that requires several staple cartridges to be used in order to staple and incise the entire path. In such instances, a first staple cartridge is seated in the cartridge jaw of a stapling instrument, the end effector of the stapling instrument is inserted into the patient, the patient tissue is clamped between the anvil jaw and the first staple cartridge, and then the first staple cartridge is fired to staple and cut the patient tissue along a first firing length. At such point, the stapling instrument is removed, the now-spent first staple cartridge is removed from the cartridge jaw, a second staple cartridge is seated in the cartridge jaw, and then the end effector is re-inserted into the patient. At such point, the end effector is aligned with the patient tissue such that the end effector can be clamped onto the patient tissue at the end of the first firing length. The second staple cartridge is then fired to staple and cut the patient tissue along a second firing length that continues the treatment path through the patient tissue. In some instances, the second firing length is sufficient to complete the treatment path but, in other instances, one or more additional staple cartridges are needed to complete the path. In any event, the patient tissue at the intersection between the first firing length and the second firing length is often stapled twice-once at the distal end of the first firing length and once at the proximal end of the second firing length. In such instances, the tissue cutting knife may have to cut staples in the first firing length when the second staple cartridge is fired. In some such instances, absent more, the staples previously fired into the patient tissue can block or impede the movement of the tissue cutting knife. In various embodiments, the staples of a staple cartridge have pre-defined cutting features that can reduce the force needed for the tissue cutting knife to transect staples previously fired into the patient tissue. In at least one embodiment, a staple has a reduced cross-section that, if contacted by the tissue cutting knife, reduces the force needed to cut through the staple. In at least one such embodiment, a wire staple comprises a base, a first staple leg extending from a first end of the base, a second leg extending from a second end of the base, and one or more recesses defined in the base, the first leg, and/or the second leg. In at least one embodiment, a staple has a weakened section that reduces the force needed to cut through the staple. In at least one such embodiment, the weakened section has been strain hardened using one or more manufacturing processes and, as a result, the weakened section is brittle and can break when the staple is cut by the tissue cutting knife.

In various embodiments, further to the above, a staple cartridge can comprise a cluster of weaker staples stored in the distal end and/or in the proximal end of the staple pattern of the staple cartridge. The staples intermediate the distal end and the proximal end, in such embodiments, are stronger than the weaker staples at the distal and/or proximal ends. Further to the above, in at least one embodiment, the weaker staples have reduced cross-sections and/or brittle cross-sections, for example, that the stronger staples do not have. After the staples of such a staple cartridge have been deployed into the patient tissue, a cluster of weaker staples, if overlapped by staples from another staple cartridge, can be sufficiently strong enough to secure the patient tissue in a sealed, or substantially sealed, state but sufficiently weak enough to be cut by the tissue cutting knife, or otherwise broken by the tissue cutting knife, without significantly increasing the force needed to move the tissue cutting knife through its staple firing stroke. In at least one embodiment, the two distal-most staples in each longitudinal row of staples in a staple cartridge comprise weaker staples, for example. Likewise, in at least one embodiment, the two proximal-most staples in each longitudinal row of staples comprise weaker staples.

In various embodiments, as discussed above, a controller of a surgical stapling instrument, such as the controller 10033, for example, is configured to control the operation of the surgical stapling instrument. As also discussed above, the surgical stapling instrument can comprise one or more motors to operate the surgical stapling instrument and drive the functions of the surgical stapling instrument such as, for example, closing the end effector of the surgical stapling instrument and advancing a firing driver through a staple firing stroke to staple and incise patient tissue. In various embodiments, the staple firing system of the surgical stapling instrument comprises an electric motor, i.e., a firing motor, that is controlled by the controller 10033. The controller 10033 comprises a pulse width modulation circuit and/or a frequency modulation circuit configured to control the speed of the firing motor. The pulse width modulation circuit is configured to control the length of the voltage pulses supplied to the firing motor to control the speed of the firing motor. In general, the pulse width modulation circuit increases the length of the voltage pulses to speed up the firing motor and decreases the length of the voltage pulses to slow down the firing motor. The frequency modulation circuit is configured to control the frequency of the voltage pulses supplied to the firing motor to control the speed of the firing motor. In general, the frequency modulation circuit increases the frequency of the voltage pulses to speed up the firing motor and decreases the frequency of the voltage pulses to slow down the firing motor.

In various instances, further to the above, the firing driver may slow down when passing through thick patient tissue and/or dense patient tissue. In various algorithms implemented by the controller 10033, the controller 10033 is configured to evaluate the speed of the firing motor during the staple firing stroke and compare the measured speed to a target speed. In at least one embodiment, the surgical stapling instrument comprises an encoder and/or a tachometer, for example, that is in communication with the controller 10033 that is used to measure the speed of the firing motor. In at least one algorithm implemented by the controller 10033, the controller 10033 increases the speed of the firing motor and/or decreases the speed of the firing motor to match the target speed. When implementing at least one such algorithm, however, the controller 10033 decreases the speed of the firing motor when the current to the firing motor exceeds one or more thresholds. If the current to the firing motor exceeds a certain threshold, the controller 10033 pauses the staple firing stroke by not applying a voltage polarity to the firing motor. In this algorithm, the controller 10033 recommences the staple firing stroke after a period of time has elapsed and/or after one or more measured parameters indicates to the controller 10033 that the staple firing stroke can be recommenced. In at least one embodiment, a processor of the controller 10033 comprises a clock and/or the controller 10033 comprises a timer circuit that can be used to evaluate whether the predetermined period of time has elapsed.

The above being said, the controller 10033 is configured to implement more than one algorithm—either simultaneously or in the alternative. In at least one embodiment, for instance, the controller 10033 is configured to implement a first algorithm if the staple cartridge seated in the end effector of the surgical stapling instrument has an implantable adjunct releasably attached to the deck of the staple cartridge and, in the alternative, a second algorithm—that is different than the first algorithm—if the staple cartridge seated in the end effector does not have an implantable adjunct releasably attached to the deck of the staple cartridge. In various embodiments, an implantable adjunct includes, for example, any of the implantable adjuncts disclosed herein. In at least one embodiment, an implantable adjunct comprises a piece of buttress material releasably adhered to the deck of the staple cartridge. In various embodiments, the firing driver of the surgical stapling instrument comprises a knife that transects the adjunct as it incises the patient tissue. In at least one embodiment, the firing driver pushes a knife distally that transects the adjunct and the patient tissue during the staple firing stroke.

In various embodiments, further to the above, the buttress releasably attached to the deck of the staple cartridge comprises a proximal end, a distal end, a deck-facing surface that faces the deck of the staple cartridge, and a tissue-facing surface that faces away from the deck of the staple cartridge. In at least one embodiment, the buttress comprises a consistent cross-sectional thickness between the deck-facing surface and the tissue-facing surface and between the proximal end and the distal end of the buttress. In at least one such embodiment, a consistent cross-sectional thickness comprises a nominal thickness and a tolerance that varies between +10% thicker and −10% thinner of the nominal thickness. In at least one embodiment, the buttress comprises a consistent density between the deck-facing surface and the tissue-facing surface and between the proximal end and the distal end of the buttress. In at least one such embodiment, a consistent density comprises a nominal density and a tolerance that varies between +10% more dense and −10% less dense of the nominal density. In at least one embodiment, the buttress comprises a consistent cross-sectional thickness and a consistent density between the deck-facing surface and the tissue-facing surface and between the proximal end and the distal end of the buttress. The cutting force needed to cut such a buttress during the staple firing stroke can be consistent, or at least substantially consistent, throughout the staple firing stroke. In other embodiments, the buttress has an inconsistent cross-sectional thickness and/or an inconsistent density. The cutting force needed to cut such a buttress during the staple firing stroke can be inconsistent during the staple firing stroke resulting in force pulses in the firing drive and/or current pulses in the current to the firing motor during the staple firing stroke. In various embodiments, as discussed below, the controller 10033 is configured to implement a first algorithm if the buttress has a consistent cross-section along the longitudinal length thereof and, in the alternative, a second, or different, algorithm if the buttress has an inconsistent cross-section along the longitudinal length thereof. In various embodiments, as discussed below, the controller 10033 is configured to implement the first algorithm if the buttress has a consistent density along the longitudinal length thereof and, in the alternative, the second algorithm if the buttress has an inconsistent density along the longitudinal length thereof.

In the second algorithm, further to the above, the controller 10033 adaptively controls the speed of the firing member during the staple firing stroke. For instance, the controller 10033 slows down and/or pauses the staple firing stroke when the current to the firing motor exceeds a threshold. In the first algorithm, however, the controller 10033 does not adaptively control the speed of the firing member during the staple firing stroke. More specifically, the controller 10033 does not slow down and/or pause the staple firing stroke when the current to the firing motor exceeds a threshold. In at least one embodiment, the controller 10033 increases the speed of the staple firing stroke when implementing the first algorithm. In such instances, as a result, the controller 10033 increases the speed of the staple firing stroke during the staple firing stroke when the buttress has a consistent thickness and/or density along the path of the firing driver and/or knife pushed distally by the firing driver. In at least one embodiment, the firing driver speeds up as the staple firing stroke progresses. In at least one such embodiment, the firing driver reaches its peak speed just before the end of the staple firing stroke wherein the firing motor is actively braked by the controller 10033 using the current and/or voltage supplied to the firing motor to stop the firing driver before it contacts the distal end of the longitudinal slot in the cartridge body. The above being said, the controller 10033 can be configured to implement any suitable algorithm, or select from a plurality of algorithms, depending on the buttress, or adjunct, being implanted.

In various embodiments, further to the above, a staple cartridge comprises an implantable adjunct and an RFID tag and a surgical stapling instrument comprises an RFID tag reader configured to read the RFID tag of the staple cartridge. In at least one embodiment, the RFID tag comprises a passive tag attached to the cartridge body of the staple cartridge and comprises, among other things, a substrate, an antenna, a micro chip, and a non-volatile memory. In various instances, the non-volatile memory is part of the micro chip or a separate chip in communication with the micro chip. In use, the RFID tag is energized by one or more signals emitted from the RFID tag reader of the surgical stapling instrument once the staple cartridge is seated in the surgical stapling instrument and, in response, the RFID tag emits a signal including at least one datum related to the implantable adjunct of the staple cartridge. The RFID tag reader comprises an antenna positioned and configured to receive the emitted signal from the RFID tag. The RFID tag reader antenna is in communication with the controller 10033 via a wire and/or a wireless transmitter such that the controller 10033 receives the at least one datum regarding the staple cartridge adjunct stored in the RFID tag. With the at least one datum regarding the staple cartridge adjunct stored in the RFID tag, the controller 10033 is configured to select between the first algorithm and the second algorithm to fire the staple cartridge. In addition to or in lieu of the RFID system described above, at least one datum regarding the staple cartridge adjunct can be stored in the surgical stapling instrument that is used by the controller 10033 to select between the first algorithm and the second algorithm to fire the staple cartridge. In addition to or in lieu of the above, the surgical stapling instrument comprises a transmitter configured to communicate with an off-board surgical hub that has stored therein at least one datum regarding the staple cartridge adjunct and a receiver configured to receive the at least one datum from the surgical hub. The controller 10033 is configured to use the at least one datum from the surgical hub to select between the first algorithm and the second algorithm to fire the staple cartridge. In at least one other embodiment, the decision for the surgical stapling instrument to use the first algorithm or the second algorithm is made by the off-board surgical hub and then communicate that decision to the surgical stapling instrument via a transmitter. In addition to or in lieu of the above, the controller 10033 is configured to monitor the electrical and/or mechanical parameters of the staple firing drive during the staple firing stroke and, from data obtained during the staple firing stroke regarding the staple firing drive, determine whether or not an adjunct is present on the staple cartridge, determine whether or not the adjunct has a consistent thickness and/or density, and alter one or more parameters of the staple firing stroke based on the obtained data and/or the above-made determinations.

In various embodiments, referring to FIGS. 23 and 23A, the staple cartridge 2000 comprises an electrical circuit 2318 including two contacts 2319 attached to the cartridge body 2100. The cartridge jaw 3310 comprises an electrical circuit 3318 including two contacts 3319 that is in communication with the controller 10033 of the surgical stapling instrument. When the staple cartridge 2000 is fully seated in the cartridge jaw 3310, the cartridge contacts 2319 engage the jaw contacts 3319 to place the cartridge circuit 2318 in communication with the jaw circuit 3310. One of the cartridge contacts 2319 is distal with respect to the other cartridge contact 2319 and, likewise, one of the jaw contacts 3319 is distal with respect to the other jaw contact 3319. When the staple cartridge 2000 is not fully seated in the cartridge jaw 3310, one of the cartridge contacts 2319 is not engaged with its respective jaw contact 3319. In such instances, the interface between the cartridge circuit 2318 and the jaw circuit 3318 is at least partially open—a condition that is detectable by the controller 10033. In at least one algorithm implemented by the controller 10033, the controller 10033 prevents the staple firing drive from performing the staple firing stroke when the controller 10033 detects that the staple cartridge 2000 is not fully seated in the cartridge jaw 3310. In at least one such embodiment, the staple firing drive comprises a firing trigger, such as a firing switch, for example, and the firing motor of the staple firing drive is not responsive to an input from the firing trigger until, or unless, the staple cartridge 2000 is fully seated in the cartridge jaw 3310 and both cartridge contacts 2319 are in contact with their respective jaw contacts 3319. In many instances, one of the cartridge contacts 2319 is in contact with its respective jaw contact 3319 but the other cartridge contact 2319 is not. For instance, the proximal cartridge contact 2319 may be in contact with the proximal jaw contact 3319 but the distal cartridge contact 2319 may not be in contact with the distal jaw contact 3319. Sensing this partial seating of the staple cartridge 2000 in the cartridge jaw 3310, in various embodiments, the controller 10033 is configured to notify the user of the surgical stapling instrument that the distal end of the staple cartridge 2000 is not seated in the cartridge jaw 3310. In at least one such embodiment, the surgical stapling instrument comprises a screen, for example, in communication with the controller 10033 that is configured to display such information. In other instances, the distal cartridge contact 2319 is in contact with the distal jaw contact 3319 but the proximal cartridge contact 2319 is not in contact the proximal jaw contact 3319. Sensing this partial seating of the staple cartridge 2000 in the cartridge jaw 3310, in various embodiments, the controller 10033 is configured to notify the user of the surgical stapling instrument that the proximal end of the staple cartridge 2000 is not seated in the cartridge jaw 3310 using, for instance, the screen of the surgical stapling instrument. Once the staple cartridge 2000 is fully seated in the cartridge jaw, in either instance, the controller 10033 is configured to notify the user that the staple cartridge 2000 is fully seated using, for instance, the screen of the surgical stapling instrument and then permit the firing motor to be responsive to an input from the firing trigger of the staple firing drive-assuming that all of the other algorithm's criteria for operating the firing motor have been met.

Further to the above, the controller 10033 is configured to use the cartridge contacts 2319 and the jaw contacts 3319 to assess the proximal-distal tilt of an unseated staple cartridge 2000. In addition to or in lieu of the above, the cartridge contacts 2319 and the jaw contacts 3319 are configured such that the controller 10033 can assess the lateral tilt of an unseated staple cartridge 2000. In various embodiments, the staple cartridge 2000 comprises a first cartridge contact 2319 on a first lateral side of the cartridge body 2100 and a second cartridge contact 2319 on a second, or opposite, lateral side of the cartridge body 2100. Similarly, the cartridge jaw 3310 comprises a first jaw contact 3319 on a first lateral sidewall 3315 of the cartridge jaw 3310 and a second jaw contact 3319 on a second lateral sidewall 3315 of the cartridge jaw 3310 opposite the first jaw contact 3319. When the staple cartridge 2000 is seated in the cartridge jaw 3310, the first cartridge contact 2319 is in contact with the first jaw contact 3319 and the second cartridge contact 2319 is in contact with the second jaw contact 3319 such that the jaw circuit 3318 is in communication with cartridge circuit 2318. In such instances, the controller 10033 is configured to determine that the staple cartridge 2000 is not laterally tilted in the cartridge jaw 3310 and permit the firing motor of the staple firing drive to be responsive to an input from the firing trigger of the staple firing drive-assuming that all of the other algorithm's criteria for operating the firing motor have been met. When the staple cartridge 2000 is not seated in the cartridge jaw 3310 and is instead tilted laterally in the cartridge jaw 3310, one of the first and second cartridge contacts 2319 will not be in contact with its respective jaw contact 3319. In such instances, the interface between the cartridge circuit 2318 and the jaw circuit 3318 is at least partially open—a condition that is detectable by the controller 10033. In at least one algorithm implemented by the controller 10033, the controller 10033 prevents the staple firing drive from performing the staple firing stroke when the controller 10033 detects that the staple cartridge 2000 is laterally tilted in the cartridge jaw 3310. In at least one such embodiment, the firing motor of the staple firing drive is not responsive to an input from the firing trigger until, or unless, the staple cartridge 2000 is fully seated in the cartridge jaw 3310 and all of the cartridge contacts 2319 are in contact with their respective jaw contacts 3319. In many instances, one of the first and second lateral cartridge contacts 2319 is in contact with its respective jaw contact 3319 but the other first and second lateral cartridge contacts 2319 is not. For instance, the first lateral cartridge contact 2319 may be in contact with the first lateral jaw contact 3319 but the second lateral cartridge contact 2319 may not be in contact with the second lateral jaw contact 3319. Sensing this partial seating of the staple cartridge 2000 in the cartridge jaw 3310, in various embodiments, the controller 10033 is configured to notify the user of the surgical stapling instrument that the second lateral side of the staple cartridge 2000 is not seated in the cartridge jaw 3310 via, for example, the screen of the surgical stapling instrument and prevent the firing motor from responding to inputs from the firing trigger. In other instances, the second lateral cartridge contact 2319 is in contact with the second lateral jaw contact 3319 but the first lateral cartridge contact 2319 is not in contact the first lateral jaw contact 3319. Sensing this partial seating of the staple cartridge 2000 in the cartridge jaw 3310, in various embodiments, the controller 10033 is configured to notify the user of the surgical stapling instrument that the first lateral side of the staple cartridge 2000 is not seated in the cartridge jaw 3310 using, for instance, the screen of the surgical stapling instrument and prevent the firing motor from responding to inputs from the firing trigger. Once the staple cartridge 2000 is fully seated in the cartridge jaw, in either instance, the controller 10033 is configured to notify the user that the staple cartridge 2000 is fully seated using, for instance, the screen of the surgical stapling instrument and then permit the firing motor to be responsive to an input from the firing trigger of the staple firing drive-assuming that all of the other algorithm's criteria for operating the firing motor have been met.

In addition to or in lieu of the above, the cartridge circuit 2318 and the jaw circuit 3318 can be used by the controller 10033 to assess the authenticity of the staple cartridge seated in the cartridge jaw 2310. If a staple cartridge 2000 is seated in the cartridge jaw 2310, the controller 10033 can interface with the cartridge circuit 2318 via the jaw circuit 3318. The cartridge circuit 2318 completes the continuity of the jaw circuit 3318 and, when implementing at least one algorithm, the controller 10033 places a voltage potential across the jaw circuit 3318 to evaluate whether current flows through the cartridge circuit 2318 and the jaw circuit 3318. If a staple cartridge other than the staple cartridge 2000 is seated in the cartridge jaw 2310 that doesn't have the cartridge circuit 2318, the jaw circuit 3318 may be open and the controller 10033 can assess that the staple cartridge seated in the cartridge jaw 3310 is not a staple cartridge 2000. According to at least one algorithm implemented by the controller 10033, the controller 10033 can determine that the seated staple cartridge is not a staple cartridge 2000, or is not authentic, and notify the user of the surgical stapling instrument, via the screen of the surgical stapling instrument, for instance, that the seated staple cartridge may not be the correct staple cartridge and/or not an authentic staple cartridge, i.e., a staple cartridge paired with the surgical stapling instrument by the manufacturer of the surgical stapling instrument. When implementing at least one such algorithm, the controller 10033 permits the firing motor to respond to an input from the firing trigger of the staple firing driver even if the staple cartridge is determined not to be authentic. When implementing at least one such algorithm, however, the controller 10033 permits the firing motor to respond to an input from the firing trigger of the staple firing driver but at a slower maximum speed and/or at a slower maximum current to the firing motor. When implementing at least one other algorithm, on the other hand, the controller 10033 does not permit the firing motor to respond to an input from the firing trigger if the controller 10033 determines that the staple cartridge seated in the cartridge jaw 3310 is not the correct staple cartridge and/or not an authentic staple cartridge.

In various embodiments, further to the above, the cartridge circuit 2318 extends through the staple cartridge 2000. In at least one embodiment, the cartridge circuit 2318 comprises a first cartridge contact 2319, a second cartridge contact 2319, and a conductor extending through the cartridge body 2100 that electrically connects the first cartridge contact 2319 and the second cartridge contact 2319. In at least one embodiment, the cartridge circuit 2318 extends through at least a portion of the sled movably positioned in the cartridge body 2100. In at least one such embodiment, the sled is at least partially comprised of metal, such as stainless steel, for example, that is part of the cartridge circuit 2318. In at least one such embodiment, the first cartridge contact 2319 is on a first lateral side of the cartridge body 2100, the second cartridge contact 2319 is on a second, or opposite, lateral side of the cartridge body 2100, and the cartridge circuit 2318 extends through the sled when the sled is in its proximal unfired position. When the sled is advanced distally, either during the staple firing stroke or unintentionally while the clinician is handling the staple cartridge 2100, the sled no longer electrically connects the first cartridge contact 2319 and the second cartridge contact 2319 and, in such instances, the cartridge circuit 2318 within the staple cartridge 2100 is open—a condition which is detectable by the controller 10033 via the jaw circuit 3318. If the controller 10033 detects that the sled is in its proximal unfired position when the firing trigger is actuated to initiate the staple firing stroke, the controller 10033 permits the firing motor of the staple firing drive to be responsive to the firing trigger actuation—assuming that all of the other algorithm's criteria for operating the firing motor have been met. If, however, the controller 10033 detects that the sled is not in its proximal unfired position when the firing trigger is actuated to initiate the staple firing stroke, the controller 10033 enters into a lockout state and prevents the firing motor from being responsive to the firing trigger actuation.

In addition to or in lieu of the above, the staple cartridge 2000 and the end effector 3300 comprise physical, mechanical, and/or geometric interfaces that retain the staple cartridge 2000 in the end effector 3300, authenticate the staple cartridge 2000, prevent the staple cartridge 2000 from being fired if the staple cartridge 2000 is not seated in the end effector 3300, prevent the staple cartridge 2000 from being fired if the staple cartridge 2000 has been previously fired, and allow an unspent staple cartridge 2000 seated in the end effector 3300 to be fired. In various embodiments, a combination of parts within the staple cartridge 2000 and/or end effector 3300 are required to co-operate to close the anvil jaw 3320 of the end effector 3300, couple the tissue cutting knife with the staple firing drive of the surgical stapling instrument, detect the presence of the sled in its unfired position in the staple cartridge prior to the staple firing stroke, advance the tissue cutting knife and the sled through the staple firing stroke, leave the sled at the distal end of the staple cartridge at the end of the staple firing stroke, decouple the tissue cutting knife from the staple firing drive, and release the staple cartridge 2000 from the cartridge jaw 3310. Such mechanical interfaces include the anvil-tissue cutting knife interface, the firing driver-tissue cutting knife interface, the firing driver-sled interface, the firing driver-cartridge body interface, the cartridge body-anvil interface, the sled-cartridge body interface, the tissue cutting knife-cartridge body interface, the tissue cutting knife-sled interface, and/or combinations thereof.

Many of the surgical instrument systems described herein are motivated by an electric motor; however, the surgical instrument systems described herein can be motivated in any suitable manner. In various instances, the surgical instrument systems described herein can be motivated by a manually-operated trigger, for example. In certain instances, the motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

The surgical instrument systems described herein have been described in connection with the deployment and deformation of staples; however, the embodiments described herein are not so limited. Various embodiments are envisioned which deploy fasteners other than staples, such as clamps or tacks, for example. Moreover, various embodiments are envisioned which utilize any suitable means for sealing tissue. For instance, an end effector in accordance with various embodiments can comprise electrodes configured to heat and seal the tissue. Also, for instance, an end effector in accordance with certain embodiments can apply vibrational energy to seal the tissue.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Patent No. D974,560, entitled STAPLE CARTRIDGE, which issued on Jan. 3, 2023, U.S. Design Pat. No. D967,421, entitled STAPLE CARTRIDGE, which issued on Oct. 18, 2022, U.S. Design Pat. No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006; U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

1. A method of assembling a surgical staple cartridge, comprising:
attaching a pan to a cartridge body, the cartridge body comprising a proximal end, a distal end, a deck, longitudinal rows of staple cavities extending between the proximal end and the distal end, and a longitudinal slot extending from the proximal end toward the distal end;
positioning staples in the staple cavities;
positioning a support in the longitudinal slot such that an end of the support extends above the deck; and
positioning a sled in the cartridge body proximal to the support such that the sled is longitudinally aligned with the support.

2. The method of claim 1, wherein the longitudinal slot comprises a longitudinal slot length, and wherein positioning the support comprises positioning the support at a midpoint of the longitudinal slot length.

3. The method of claim 1, wherein the longitudinal slot comprises a longitudinal slot length, and wherein positioning the support comprises positioning the support proximal to a midpoint of the longitudinal slot length.

4. The method of claim 1, wherein the longitudinal slot comprises a longitudinal slot length, and wherein positioning the support comprises positioning the support distal to a midpoint of the longitudinal slot length.

5. The method of claim 1, wherein the longitudinal rows of staple cavities define a longitudinal staple field length, and wherein positioning the support comprises positioning the support at a midpoint of the longitudinal staple field length.

6. The method of claim 1, wherein the longitudinal rows of staple cavities define a longitudinal staple field length, and wherein positioning the support comprises positioning the support proximal to a midpoint of the longitudinal staple field length.

7. The method of claim 1, wherein the longitudinal rows of staple cavities define a longitudinal staple field length, and wherein positioning the support comprises positioning the support distal to a midpoint of the longitudinal staple field length.

8. The method of claim 1, wherein positioning the support comprises positioning a portion of the support between the cartridge body and the pan.

9. The method of claim 1, wherein the longitudinal slot is defined by a first lateral sidewall and a second lateral sidewall, further comprising attaching a first support plate to the first lateral sidewall and attaching a second support plate to the second lateral sidewall.

10. The method of claim 1, wherein the longitudinal slot is defined by a first lateral sidewall and a second lateral sidewall, further comprising embedding a first support plate in the first lateral sidewall and embedding a second support plate in the second lateral sidewall.

11. A method of assembling a staple cartridge for use with a surgical stapling instrument, comprising:
obtaining a cartridge body configured to be seated in a jaw of the surgical stapling instrument, the cartridge body comprising a proximal end, a distal end, a deck, longitudinal rows of staple cavities extending between the proximal end and the distal end, and a longitudinal slot extending from the proximal end toward the distal end;
positioning a longitudinally-slideable support in the longitudinal slot such that an end of the support extends above the deck; and
positioning a longitudinally-slideable sled in the cartridge body proximal to the support such that the sled contacts the support during a staple firing stroke and moves the support toward the distal end during the staple firing stroke.

12. The method of claim 11, wherein the longitudinal slot comprises a longitudinal slot length, and wherein positioning the support comprises positioning the support at a midpoint of the longitudinal slot length.

13. The method of claim 11, wherein the longitudinal slot comprises a longitudinal slot length, and wherein positioning the support comprises positioning the support proximal to a midpoint of the longitudinal slot length.

14. The method of claim 11, wherein the longitudinal slot comprises a longitudinal slot length, and wherein positioning the support comprises positioning the support distal to a midpoint of the longitudinal slot length.

15. The method of claim 11, wherein the longitudinal rows of staple cavities define a longitudinal staple field length, and wherein positioning the support comprises positioning the support at a midpoint of the longitudinal staple field length.

16. The method of claim 11, wherein the longitudinal rows of staple cavities define a longitudinal staple field length, and wherein positioning the support comprises positioning the support proximal to a midpoint of the longitudinal staple field length.

17. The method of claim 11, wherein the longitudinal rows of staple cavities define a longitudinal staple field length, and wherein positioning the support comprises positioning the support distal to a midpoint of the longitudinal staple field length.

18. The method of claim 11, further comprising attaching a pan to the cartridge body, wherein positioning the support comprises positioning a portion of the support between the cartridge body and the pan.

19. The method of claim 11, wherein the longitudinal slot is defined by a first lateral sidewall and a second lateral sidewall, further comprising attaching a first support plate to the first lateral sidewall and attaching a second support plate to the second lateral sidewall.

20. The method of claim 11, wherein the longitudinal slot is defined by a first lateral sidewall and a second lateral sidewall, further comprising embedding a first support plate in the first lateral sidewall and embedding a second support plate in the second lateral sidewall.
